SEQUENCE LISTING (126 sequences)

SEQ ID NO: 1 - DNA, 3261 bp, Homo sapiens
  CDS (1)..(2853)
  misc_feature (3195): n is any nucleic acid
  misc_feature (3248): n is any nucleic acid
  misc_feature (3255): n is any nucleic acid
  misc_feature (3261): n is any nucleic acid
atggggaacgcggagcgggctccggggtctcggagctttgggcccgtacccacgctgctgctgctcgccgcggcgctactggccgtgtcggacgcactcgggcgcccctccgaggaggacgaggagctagtggtgccggagctggagcgcgccccgggacacgggaccacgcgcctccgcctgcacgcctttgaccagcagctggatctggagctgcggcccgacagcagctttttggcgcccggcttcacgctccagaacgtggggcgcaaatccgggtccgagacgccgcttccggaaaccgacctggcgcactgcttctactccggcaccgtgaatggcgatcccagctcggctgccgccctcagcctctgcgagggcgtgcgcggcgccttctacctgctgggggaggcgtatttcatccagccgctgcccgccgccagcgagcgcctcgccaccgccgccccaggggagaagccgccggcaccactacagttccacctcctgcggcggaatcggcagggcgacgtaggcggcacgtgcggggtcgtggacgacgagccccggccgactgggaaagcggagaccgaagacgaggacgaagggactgagggcgaggacgaagggcctcagtggtcgccgcaggacccggcactgcaaggcgtaggacagcccacaggaactggaagcataagaaagaagcgatttgtgtccagtcaccgctatgtggaaaccatgcttgtggcagaccagtcgatggcagaattccacggcagtggtctaaagcattaccttctcacgttgttttcggtggcagccagattgtacaaacaccccagcattcgtaattcagttagcctggtggtggtgaagatcttggtcatccacgatgaacagaaggggccggaagtgacctccaatgctgccctcactctgcggaacttttgcaactggcagaagcagcacaacccacccagtgaccgggatgcagagcactatgacacagcaattcttttcaccagacaggacttgtgtgggtcccagacatgtgatactcttgggatggctgatgttggaactgtgtgtgatccgagcagaagctgctccgtcatagaagatgatggtttacaagctgccttcaccacagcccatgaattaggccacgtgtttaacatgccacatgatgatgcaaagcagtgtgccagccttaatggtgtgaaccaggattcccacatgatggcgtcaatgctttccaacctggaccacagccagccttggtctccttgcagtgcctacatgattacatcatttctggataatggtcatggggaatgtttgatggacaagcctcagaatcccatacagctcccaggcgatctccctggcacctcgtacgatgccaaccggcagtgccagtttacatttggggaggactccaaacactgccctgatgcagccagcacatgtagcaccttgtggtgtaccggcacctctggtggggtgctggtgtgtcaaaccaaacacttcccgtgggcggatggcaccagctgtggagaagggaaatggtgtatcaacggcaagtgtgtgaacaaaaccgacagaaagcattttgatacgccttttcatggaagctggggaatgtgggggccttggggagactgttcgagaacgtgcggtggaggagtccagtacacgatgagggaatgtgacaacccagtcccaaagaatggagggaagtactgtgaaggcaaacgagtgcgctacagatcctgtaaccttgaggactgtccagacaataatggaaaaacctttagagaggaacaatgtgaagcacacaacgagttttcaaaagcttcctttgggagtgggcctgcggtggaatggattcccaagtacgctggcgtctcaccaaaggacaggtgcaagctcatctgccaagccaaaggcattggctacttcttcgttttgcagcccaaggttgtagatggtactccatgtagcccagattccacctctgtctgtgtgcaaggacagtgtgtaaaagctggttgtgatcgcatcatagactccaaaaagaagtttgataaatgtggtgtttgcgggggaaatggatctacttgtaaaaaaatatcaggatcagttactagtgcaaaacctggatatcatgatatcatcacaattccaactggagccaccaacatcgaagtgaaacagcggaaccagaggggatccaggaacaatggcagctttcttgccatcaaagctgctgatggcacatatattcttaatggtgactacactttgtccaccttagagcaagacattatgtacaaaggtgttgtcttgaggtacagcggctcctctgcggcattggaaagaattcgcagctttagccctctcaaagagcccttgaccatccaggttcttactgtgggcaatgcccttcgacctaaaattaaatacacctacttcgtaaagaagaagaaggaatctttcaatgctatccccactttttcagcatgggtcattgaagagtggggcgaatgttctaagtcatgtgaattgggttggcagagaagactggtagaatgccgagacattaatggacagcctgcttccgagtgtgcaaaggaagtgaagccagccagcaccagaccttgtgcagaccatccctgcccccagtggcagctgggggagtggtcatcatgttctaagacctgtgggaagggttacaaaaaaagaagcttgaagtgtctgtcccatgatggaggggtgttatctcatgagagctgtgatcctttaaagaaacctaaacatttcatagacttttgcacaatggcagaatgcagttaagtggtttaagtggtgttagctttgaggcaaggcaaagtgaggaagggctggtgcagggaaagcaagaaggctggagggatccagcgtatcttgccagtaaccagtgaggtgtatcagtaaggtgggattatgggggtagatagaaaaggagttgaatcatcagagtaaactgccagttgcaaatttgataggatagttagtgaggattattaacctctgagcagtgatatagcataataaanccccgggcattattattattatttcttttgttacatctattacaagtttagaaaaaacaaagcaattgtcaaaaaaaaaaaaaaaaaaaaaaaaaaaaaaagggcggccgctctagaggatccctcgaggggcccaagcttacgcgtgcatgntgtcatnagtctn

SEQ ID NO: 2 - protein, 950 aa, Homo sapiens
MGNAERAPGSRSFGPVPTLLLLAAALLAVSDALGRPSEEDEELVVPELERAPGHGTTRLRLHAFDQQLDLELRPDSSFLAPGFTLQNVGRKSGSETPLPETDLAHCFYSGTVNGDPSSAAALSLCEGVRGAFYLLGEAYFIQPLPAASERLATAAPGEKPPAPLQFHLLRRNRQGDVGGTCGVVDDEPRPTGKAETEDEDEGTEGEDEGPQWSPQDPALQGVGQPTGTGSIRKKRFVSSHRYVETMLVADQSMAEFHGSGLKHYLLTLFSVAARLYKHPSIRNSVSLVVVKILVIHDEQKGPEVTSNAALTLRNFCNWQKQHNPPSDRDAEHYDTAILFTRQDLCGSQTCDTLGMADVGTVCDPSRSCSVIEDDGLQAAFTTAHELGHVFNMPHDDAKQCASLNGVNQDSHMMASMLSNLDHSQPWSPCSAYMITSFLDNGHGECLMDKPQNPIQLPGDLPGTSYDANRQCQFTFGEDSKHCPDAASTCSTLWCTGTSGGVLVCQTKHFPWADGTSCGEGKWCINGKCVNKTDRKHFDTPFHGSWGMWGPWGDCSRTCGGGVQYTMRECDNPVPKNGGKYCEGKRVRYRSCNLEDCPDNNGKTFREEQCEAHNEFSKASFGSGPAVEWIPKYAGVSPKDRCKLICQAKGIGYFFVLQPKVVDGTPCSPDSTSVCVQGQCVKAGCDRIIDSKKKFDKCGVCGGNGSTCKKISGSVTSAKPGYHDIITIPTGATNIEVKQRNQRGSRNNGSFLAIKAADGTYILNGDYTLSTLEQDIMYKGVVLRYSGSSAALERIRSFSPLKEPLTIQVLTVGNALRPKIKYTYFVKKKKESFNAIPTFSAWVIEEWGECSKSCELGWQRRLVECRDINGQPASECAKEVKPASTRPCADHPCPQWQLGEWSSCSKTCGKGYKKRSLKCLSHDGGVLSHESCDPLKKPKHFIDFCTMAECS

SEQ ID NO: 3 - DNA, 3008 bp, Homo sapiens
  CDS (1)..(2670)
  Misc_feature (2887): N is any nucleic acid
  Misc_feature (2957): N is any nucleic acid
  Misc_feature (2970): N is any nucleic acid
  Misc_feature (2981): N is any nucleic acid
atgttccccgcccccgccgccccccggtggcttccgttcctgctgctgctgctgctgctgctgctgccgctggcccgcggcgccccggcccggcccgcagccggggggcaggcctcggagctggtggtgcccacgcggttgcccggcagcgcgggcgagctcgcgctccacctgtccgccttcggcaagggcttcgtgttgcgcctggcgcccgacgacagcttcctggcgcccgagttcaagatcgagcgcctcgggggctccggccgggcgaccgggggcgagcgggggctgcgcggctgttttttttccggcaccgtgaatggggagcccgagtcgctggcggcggtcagcctgtgccgcgggctgagcggctccttcctgctggacggcgaggagttcaccatccagccgcagggcgcggggggctccctggctcagccgcaccgcctgcagcgctggggtcccgccggagcccgccccctcccgcgaggacccgagtgggaggtggagacgggagagggtcagaggcaggagagaggagaccaccaggaggacagcgaggaggagagccaagaagaggaggcagaaggcgctagcgagccgccaccgcccctgggggccacgagtaggaccaagcggtttgtgtctgaggcgcgcttcgtggagacgctgctggtggccgatgcgtccatggctgccttctacggggccgacctgcagaaccacatcctgacgttaatgtctgtggcagcccgaatctacaagcaccccagcatcaagaattccatcaacctgatggtggtaaaagtgctgatcgtagaagatgaaaaatggggcccagaggtgtccgacaatggggggcttacactgcgtaacttctgcaactggcagcggcgtttcaaccagcccagcgaccgccacccagagcactacgacacggccatcctgctcaccagacagaacttctgtgggcaggaggggctgtgtgacaccctgggtgtggcagacatcgggaccatttgtgaccccaacaaaagctgctccgtgatcgaggatgaggggctccaggcggcccacaccctggcccatgaactagggcacgtcctcagcatgccccacgacgactccaagccctgcacacggctcttcgggcccatgggcaagcaccacgtgatggcaccgctgttcgtccacctgaaccagacgctgccctggtccccctgcagcgccatgtatctcacagagcttctggacggcgggcacggagactgtctcctggatgcccctggtgcggccctgcccctccccacaggcctcccgggccgcatggccctgtaccagctggaccagcagtgcaggcagatctttgggccggatttccgccactgccccaacacctctgctcaggacgtctgcgcccagctttggtgccacactgatggggctgagcccctgtgccacacgaagaatggcagcctgccctgggctgacggcacgccgtgcgggcctgggcacctctgctcagaaggcagctgtctacctgaggaggaagtggagaggcccaagcccgtggtagatggaggctgggcaccgtggggaccctggggagaatgttctcggacctgtggaggaggagtacagttttcacaccgtgagtgcaaggaccccgagcctcagaatggaggaagatactgcctgggtcggagagccaagtaccagtcatgccacacggaggaatgcccccctgacgggaaaagcttcagggagcagcagtgtgagaagtataatgcctacaattacactgacatggacgggaatctcctgcagtgggtccccaagtatgctggggtgtccccccgggaccgctgcaagttgttctgccgagcccgggggaggagcgagttcaaagtgttcgaggccaaggtgattgatggcaccctgtgtgggccagaaacactggccatctgtgtccgtggccagtgtgtcaaggccggctgtgaccatgtggtggactcgcctcggaagctggacaaatgcggggtgtgtgggggcaaaggcaactcctgcaggaaggtctccgggtccctcacccccaccaattatggctacaatgacattgtcaccatcccagctggtgccactaatattgacgtgaagcagcggagccacccgggtgtgcagaacgatgggaactacctggcgctgaagacggctgatgggcagtacctgctcaacggcaacctggccatctctgccatagagcaggacatcttggtgaaggggaccatcctgaagtacagcggctccatcgccaccctggagcgcctgcagagcttccggcccttgccagagcctctgacagtgcagctcctgacagtccctggcgaggtcttccccccaaaagtcaaatacaccttctttgttcctaatgacgtggactttagcatgcagagcagcaaagagagagcaaccaccaacatcatccagccgctgctccacgcacagtgggtgctgggggactggtctgagtgctctagcacctgcggggccggctggcagaggcgaactgtagagtgcagggacccctccggccaggcctctgccacctgcaacaaggctctgaaacccgaggatgccaagccctgcgaaagccagctgtgccccctgtgattcaggggggcaggggccagtcttgtgctcctggacatgcggtactgaggtgcagacaaggtctccactgtggtgactgggtcccttggccatatcaaggcagcacggcccacccaggcctcccattgccgcaacccctccagtactgcacaaattcctaagggggaagagaaaaggtatggggcggcaaaacctatcatcaactgtccawtgnaatggaacttgctcgggttcaattaaaggcataagttaaagtaaattcattatgatcaacagacctcacntcatctgttgcangatacaactantaaaaaaaaaaaaaaaaaaaaaaaaaa

SEQ ID NO: 4 - protein, 890 aa, Homo sapiens [sequence omitted]

SEQ ID NO: 5 - protein, 1203 aa, Bovine
MDPPAGAAGRLLCPALLLLLLLPLPADARLAAAAADPPGGPQGHGAERILAVPVRTDAQGRLVSHVVSAATAPAGVRTRRAAPAQIPGLSGGSEEDPGGRLFYNVTVFGRDLHLRLRPNARLVAPGATVEWQGESGATRVEPLLGTCLYVGDVAGLAESSSVALSNCDGLAGLIRMEEEEFFIEPLEKGLAAKEAEQGRVHVVYHRPTTSRPPPLGQALDTGISADSLDSLSRALGVLEERVNSSRRRMRRHAADDDYNIEVLLGVDDSVVQFHGTEHVQKYLLTLMNIVNEIYHDESLGAHINVVLVRIILLSYGKSMSLIEIGNPSQSLENVCRWAYLQQKPDTDHDEYHDHAIFLTRQDFGPSGMQGYAPVTGMCHPVRSCTLNHEDGFSSAFVVAHETGHVLGMEHDGQGNRCGDEVRLGSIMAPLVQAAFHRFHWSRCSQQELSRYLHSYDCLRDDPFTHDWPALPQLPGLHYSMNEQCRFDFGLGYMMCTAFRTFDPCKQLWCSHPDNPYFCKTKKGPPLDGTMCAPGKHCFKGHCIWLTPDILKRDGNWGAWSPFGSCSRTCGTGVKFRTRQCDNPHPANGGRTCSGLAYDFQLCNSQDCPDALADFREEQCRQWDLYFEHGDAQHHWLPHEHRDAKERCHLYCESKETGEVVSMKRMVHDGTRCSYKDAFSLCVRGDCRKVGCDGVIGSSKQEDKCGVCGGDNSHCKVVKGTFSRSPKKLGYIKMFEIPAGARHLLIQEADTTSHHLAVKNLETGKFILNEENDVDPNSKTFIAMGVEWEYRDEDGRETLQTMGPLHGTITVLVIPEGDARISLTYKYMIHEDSLNVDDNNVLEDDSVGYEWALKKWSPCSKPCGGGSQFTKYGCRRRLDHKMVHRGFCDSVSKPKAIRRTCNPQECSQPVWVTGEWEPCSRSCGRTGMQVRSVRCVQPLHNNTTRSVHTKHCNDARPEGRRACNRELCPGRWRAGSWSQCSVTCGNGTQERPVLCRTADDSFGVCREERPETARICRLGPCPRNTSDPSKKSYVVQWLSRPDPNSPVQETSSKGRCQGDKSVFCRMEVLSRYCSIPGYNKLCCKSCNPHDNLTDVDDRAEPPSGKHNDIEELMPTLSVPTLVMEVQPPPGIPLEVPLNTSSTNATEDHPETNAVDVPYKIPGLEDEVQPPNLIPRRPSPYEKTRNQRIQELIDEMRKKEMLGKF

SEQ ID NO: 6 - protein, 50 aa, Homo sapiens
DDGWSPWSEWTSCSTSCGNGIQQRGRSCDSLNNRCEGSSVQTRTCHIQEC

SEQ ID NO: 7 - protein, 57 aa, Homo sapiens
DGGWSHWSPWSSCSVTCGDGVITRIRLCNSPSPQMNGKPCEGEARETKACKKDACPI

SEQ ID NO: 8 - protein, 57 aa, Homo sapiens
NGGWGPWSPWDICSVTCGGGVQKRSRLCNNPTPQFGGKDCVGDVTENQICNKQDCPI

SEQ ID NO: 9 - protein, 50 aa, Homo sapiens
EEGWSPWAEWTQCSVTCGSGTQQRGRSCDVTSNTCLGPSIQTRACSLSKC

SEQ ID NO: 10 - protein, 57 aa, Homo sapiens
DGGWSHWSPWSSCSVTCGVGNITRIRLCNSPVPQMGGKNCKGSGRETKACQGAPCPI

SEQ ID NO: 11 - protein, 56 aa, Homo sapiens
DGRWSPWSPWSACTVTCAGGIRERTRVCNSPEPQYGGKACVGDVQERQMCNKRSCP

SEQ ID NO: 12 - DNA, 3974 bp, Homo sapiens
ggtacctaagtgagtagggcgtccgatcgacggacgccttttttttgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcgtcgacaattcgcgcgcgaaggcgaagcggcatgcatttacgttgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgtcgaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagcttaaaaaactgcaaaaaatagtttgacttgtgagcggataacaattaagatgtacccaattgtgagcggataacaatttcacacattaaagaggagaaattacatatg

SEQ ID NO: 13 - DNA, 112 bp, Homo sapiens
aagcttaaaaaactgcaaaaaatagtttgacttgtgagcggataacaattaagatgtacccaattgtgagcggataacaatttcacacattaaagaggagaaattacatatg

SEQ ID NO: 14 - DNA, 542 bp, Mus musculus
  Misc_feature (3): N is any nucleic acid
  Misc_feature (21): N is any nucleic acid
  Misc_feature (22): N is any nucleic acid
  Misc_feature (361): N is any nucleic acid
  Misc_feature (369): N is any nucleic acid
  Misc_feature (407): N is any nucleic acid
  Misc_feature (427): N is any nucleic acid
  Misc_feature (479): N is any nucleic acid
  Misc_feature (482): N is any nucleic acid
  Misc_feature (520): N is any nucleic acid
  Misc_feature (535): N is any nucleic acid
gtncgaatttcggcacgagannttagacgccttttcatggaagctggggaatgtgggggccttggggagactgttcgagaacgtgcggtggaggagtccagtacacgatgagggaatgtgacaacccagtcccaaagaatggagggaagtactgtgaaggcaaacgagtgcgctacagatcctgtaaccttgaggactgtccagacaataatggaaaaacctttagagaggaacaatgtgaagcacacaacgagttttcaaaagcttcctttgggagtgggcctgcggtggaatggattcccaagtacgctggcgtctcaccaaaggacaggtgcaagttcatgttgccaagccaaaggcnttggctanttctttcgttttgcagcccaaggttgttaggtgggtantccatgttaggcccagattncacctttgtctgtgtgcaaggacagtgtgttaaaagttggttgtgatccgcntcntagattccaaaaggagttttgttaatgtggtgttttcngggggaatggtctanttttaaa

SEQ ID NO: 15 - DNA, 320 bp, Unknown
  Description of Unknown Organism:Unknown
cagagaacattcgccccactcttcaatgacccatgctgaaaaagtggggatagcattgaaagattccttcttcttctttacgaagtaggtgtatttaattttaggtcgaagggcattgcccacagtaagaacctggatggtcaagggctctttgagagggctaaagctgcgaattctttccaatgccgcagaggagccgctgtacctcaagacaacacctttgtacataatgtcttgctctaaggtggacaaagtgtagtcaccattaagaatatatgtgccatcagcagctttgatggcaagaaagctgcccttgttcc

SEQ ID NO: 16 - DNA, 316 bp, Eimeria tenella
aatgccgagacattaatggacagcctgcttccgagtgtgcaaaggaagtgaagccagccagcaccagaccttgtgcagaccatccctgcccccagtggcagctgggggagtggtcatcatgttctaagacctgtgggaagggttacaaaaaaagaagcttgaagtgtctgtcccatgatggaggggtgttatctcatgagagctgtgatcctttaaagaaacctaaacatttcatagacttttgcacaatggcagaatgcagttaagtggtttaagtggtgttagctttgagggcaaggcaaagtgaggaagggct

SEQ ID NO: 17 - DNA, 383 bp, Caenorhabditis elegans
  Misc_feature (160): N is any nucleic acid
  Misc_feature (326): N is any nucleic acid
  Misc_feature (358): N is any nucleic acid
  Misc_feature (366): N is any nucleic acid
  Misc_feature (377): N is any nucleic acid
  Misc_feature (379): N is any nucleic acid
gtcgacccacgcgtccggatggtactccatgtagcccagattccacctctgtctgtgtgcaaggacagtgtgtaaaagctggttgtgatcgcatcatagactccaaaaagaagtttgataaatgtggtgtttgcgggggaaatggatctacttgtaaaanaatatcaggatcagttactagtgcaaaacctgggatatcatgatatcatcacaattccaactgggagccaccaacatcgaagtgaaacagcggaaccagaggggatccagggaacaatgggcagctttcttgccatcaaagctgctggatggcacatatattcttnaatggtgactacactttgtccaccttagaganagacattntgtgacaaagngnttgt

SEQ ID NO: 18 - DNA, 404 bp, Crotalus atrox
  Misc_feature (21): N is any nucleic acid
  Misc_feature (301): N is any nucleic acid
  Misc_feature (335): N is any nucleic acid
  Misc_feature (373): N is any nucleic acid
  Misc_feature (378): N is any nucleic acid
  Misc_feature (382): N is any nucleic acid
  Misc_feature (383): N is any nucleic acid
cccacgcgtccgcccacggtnccgggacttgtgtgggtcccagacatgtgatactcttgggatggctgatgttggaactgtgtgtgatccgagcagaagctgctccgtcatagaagatgatggtttacaagctgccttcaccacagcccatgaattaggccacgtgtttaacatgccacatgatggatgcaaagcagtgtgccagccttaaatggtgtgaaccagggattcccacatgatggcgtcaatgctttccaacctgggaccacagccagccttgggtcctccttgcagtggcctnacatggattgacatcatttctgggatgaatggtncatgggggaatgttttgattggacaagccttcagaatnccctnacannttcccaggggttctccctggg

SEQ ID NO: 19 - DNA, 152 bp, Homo sapiens
  Misc_feature (105): N is any nucleic acid
  Misc_feature (122): N is any nucleic acid
  Misc_feature (135): N is any nucleic acid
atcgtagaagatgaaaaatggggcccagaggtgtccgacaatggggggcttacactgcgtaacttctgcaactggcagcggcgtttcaaccagcccagcgaccgncacccagagcactacgncacggccatcctnctcaccagacagaactt

SEQ ID NO: 20 - DNA, 4180 bp, Unknown
  Description of Unknown Organism:Unknown
gcagctccgagctaggtgctatcgcaaggccagagcgcacagcccggcggagagagcagatccttgctcagatcgagtcaaatcgggccaaggcggaggacgaagagtccaggctcctattctggacttgttccccagctccgggggcgcttctaggtcctgcagcagccagcagtgcggagccaccaactcggtgctggaatgaaaaaattcccgcgcgccagtgcagaatctttctaagtgacccggagcttcgggtgctagctctgcacgaactttcccatcaaagtgatcgtgaattttaagcatcaggagcaggccagcgaagctctacgcgtctaaacgtctatccagaccaagagttctctgcggtgcagggtgcggtgccatgcagccaaaagtcccttttggggtcacgcaagcagaagccctgctccgacatgggggacgtccagcgggcagcgagatctcggggctctctgtccgcacacatgctgttgctgctcctcgcttccataacaatgctgctatgtgcgcggggcgcacacgggcgccccacggaggaagatgaggagctggtcctgccctcgctggagcgcgccccgggccacgattccaccaccacacgccttcgtctggacgcctttggccagcagctacatctgaagttgcagccggacagcggtttcttggcgcctggcttcaccctgcagactgtggggcgcagtcccgggtccgaggcacaacatctggaccccaccggggacctggctcactgcttctactctggcacggtgaacggtgatcccggctctgccgcagccctcagcctctgtgaaggtgtgcgtggtgccttctacctacaaggagaggagttcttcattcagccagcgcctggagtggccaccgagcgcctggcccctgccgtgcccgaggaggagtcatccgcacggccgcagttccacatcctgaggcgaaggcggcggggcagtggcggcgccaagtgcggcgtcatggacgacgagaccctgccaaccagcgactcgcgacccgagagccagaacacccggaaccagtggcctgtgcgggaccccacgcctcaggacgcgggaaagccatcaggaccaggaagcataaggaagaagcgatttgtgtccagcccccgttatgtggaaaccatgctcgtagctgaccagtccatggccgacttccacggcagcggtctaaagcattaccttctaaccctgttctcggtggcagccaggttttacaagcatcccagcattaggaattcaattagcctggtggtggtgaagatcttggtcatatacgaggagcagaagggaccagaagttacctccaatgcagctctcacccttcggaatttctgcagctggcagaaacaacacaacagccccagtgaccgggatccagagcactatgacactgcaattctgttcaccagacaggatttatgtggctcccacacgtgtgacactctcggaatggcagatgttggaaccgtatgtgaccccagcaggagctgctcagtcatagaagatgatggtttgcaagccgccttcaccacagcccatgaattgggccatgtgtttaacatgccgcacgatgatgctaagcactgtgccagcttgaatggtgtgagtggcgattctcatctgatggcctcgatgctctccagcttagaccatagccagccctggtcaccttgcagtgcctacatggtcacgtccttcctagataatggacacggggaatgtttgatggacaagccccagaatccaatcaagctcccttctgatcttcccggtaccttgtacgatgccaaccgccagtgtcagtttacattcggagaggaatccaagcactgccctgatgcagccagcacatgtactaccctgtggtgcactggcacctccggtggcttactggtgtgccaaacaaaacacttcccttgggcagatggcaccagctgtggagaagggaagtggtgtgtcagtggcaagtgcgtgaacaagacagacatgaagcattttgctactcctgttcatggaagctggggaccatggggaccgtggggagactgctcaagaacctgtggtggtggagttcaatacacaatgagagaatgtgacaacccagtcccaaagaacggagggaagtactgtgaaggcaaacgagtccgctacaggtcctgtaacatcgaggactgtccagacaataacggaaaaacgttcagagaggagcagtgcgaggcgcacaatgagttttccaaagcttcctttgggaatgagcccactgtagagtggacacccaagtacgccggcgtctcgccaaaggacaggtgcaagctcacctgtgaagccaaaggcattggctactttttcgtcttacagcccaaggttgtagatggcactccctgtagtccagactctacctctgtctgtgtgcaagggcagtgtgtgaaagctggctgtgatcgcatcatagactccaaaaagaagtttgataagtgtggcgtttgtggaggaaacggttccacatgcaagaagatgtcaggaatagtcactagtacaagacctgggtatcatgacattgtcacaattcctgctggagccaccaacattgaagtgaaacatcggaatcaaagggggtccagaaacaatggcagctttctggctattagagccgctgatggtacctatattctgaatggaaacttcactctgtccacactagagcaagacctcacctacaaaggtactgtcttaaggtacagtggttcctcggctgcgctggaaagaatccgcagctttagtccactcaaagaacccttaaccatccaggttcttatggtaggccatgctctccgacccaaaattaaattcacctactttatgaagaagaagacagagtcattcaacgccattcccacattttctgagtgggtgattgaagagtggggggagtgctccaagacatgcggctcaggttggcagagaagagtagtgcagtgcagagacattaacggacaccctgcttccgaatgtgcaaaggaagtgaagccagccagtaccagaccttgtgcagaccttccttgcccacactggcaggtgggggattggtcaccatgttccaaaacttgcgggaagggttacaagaagagaaccttgaaatgtgtgtcccacgatgggggcgtgttatcaaatgagagctgtgatcctttgaagaagccaaagcattacattgacttttgcacactgacacagtgcagttaagaggcgttagaggacaaggtagcgtggggaggggctgatacactgagtgcaagagtactggagggatccagtgagtcaaaccagtaagcagtgaggtgtggcaaggaggtgtgtgtaggggatacatagcaaaggaggtagatcaggacactaccctgccagttacattctgataaggtagttaatgaggcacagtagcatctgaaagaccatacagagcactaaggagccccaaagcactattagtatctcttttcttatatctatcgcccaaataattttcagagtctggcagaagccctgttgcactgtactaactagatacttcttatcacaaagattgggaaaggcaaagcagaaagatggtaagactgggtttcaaacaaggcttggtttcaatcactggaggcaaggaggaggggacaaacaagatcattattcgaagtcgctggttgctgtggttttacggaaggttgatgcatcattcctatcaacagtgaaaagttcagcttgttcaacgtgacagaaaggctcatctccgtgaaagagctcctgatttcttcttacaccatctcagttcttaactatagttcatgttgaggtagaaacaattcatctatttataaaatgtacattggaaaaaaaaagtgaagtttatgaggtacacataaaaactgaaggaaacaatgagcaacatgcctcctgctttgcttcctcctgaggtaaacctgcctggggattgaggttgtttaagattatccatggctcacaagaggcagtaaaataatacatgttgtgccagagttagaatggggtatagagatcagggtcccatgagatggggaacatggtgatcactcatctcacatgggaggctgctgcag

SEQ ID NO: 21 - DNA, 9248 bp, Unknown
  Description of Unknown Organism:Unknown
gcagctccgagctaggtgctatcgcaaggccagagcgcacagcccggcggagagagcagatccttgctcagatcgagtcaaatcggggccaaggcggaggacgaagagtccaggctcctattctggacttgttccccagctccgggggcgcttctaggtcctgcagcagccaggagtgcggagccaccaactcggtgctggaatgaaaaaattcccgcgcgccagtgcagaatctttctaagtgacccggagcttcgggtgctagctctgcacgaactttcccatcaaagtgatcgtgaattttaagcatcaggagcaggccagcgaagctctacgcgtctaaacgtctatccagaccaagagttctctgcggtgcagggtgcggtgccatgcagccaaaagtccctttggggtcacgcaagcagaagccctgctccgacatgggggacgtccagcgggcagcgagatctcggggctctctgtccgcacacatgctgttgctgctcctcgcttccataacaatgctgctatgtgcgcggggcgcacacgggcgccccacggaggaagatgaggagctggtcctgccctcgctggagcgcgccccgggccacgattccaccaccacacgccttcgtctggacgcctttggccagcagctacatctgaagttgcagccggacagcggtttcttggcgcctggcttcaccctgcagactgtggggcgcagtcccgggtccgaggcacaacatctggaccccaccggggacctggctcactgcttctactctggcacggtgaacggtgatcccggctctgccgcagccctcagcctctgtgaaggtgtgcgtggtgccttctacctacaaggagaggagttcttcattcagccagcgcctggagtggccaccgagcgcctggcccctgccgtgcccgaggaggagtcatccgcacggccgcagttccacatcctgaggcgaaggcggcggggcagtggcggcgccaagtgcggcgtcatggacgacgagaccctgccaaccagcgactcgcgacccgagagccagaacacccggaaccagtggcctgtgcgggaccccacgcctcaggacgcgggaaagccatcaggtataagagtgacccccatctctcagtctttacgaggcgtgacttggggtcacactccagatcgcctctaaatgcgaatgactcagacttgcagtgaattgaagttctgggtcgtgaccttcccgctcccccccccccaaaaaaagtgtgaccatactctgctagaacacttatttgcccgaatagttaataatttgagaaagagagaaagaatcggaggtcctgtagataagggctaagcgtttcctccgcgaagccaataacccgactccttacactggagaatctctctccatccctttaatgcctttagtgaatgtatgagttcactttaactaggttgtagtttcgcgctgagttttgtaacgtcagtccgtgtgagcacgtagcgctcaaaggagggcggagtagaggagccatggtgacctggatgtgcgttcaggagcctgggcaacggcagtggtgatctcatttctgtggccttccgtctgtccccttcccccatttgaaaagctgaccccgatggctggtggctccgttgggcccctctgcagaacctgcttgggaggtctttgcttggttcgccccgcctccacgcgcctcctacctcggcctcgttgctcgcactccctctcccggcagaggttggactccccagcgctgtggaatgttagcctggactgatcctccctgctacacattcgcctgactctgccgtgttcagtctctaccagccagttagttctttttaatcattcaaatttctttttgcccttttctagatttctccctcttttccgacttgtccctaggagctggtattcatatcctactttacgatttctctgaccgctgagtctcagcagcccgaaaaaggccattttccaaattggcaaccctggtttgagaaaggaacttattccccccggggcactgggagtgagaggaggcaggaaaacactgctgggcagagtgggtggtcctagtgcccggaactggatcaagcagagaaccccctgggaccccttgaatgagagagctgagccttacagactgagactcctcaagccccaccccttggctgagctccccgccctgccccatgccttccacgtggagctggatgatctcattcgggatttcagccctggcttcaatagtgaaagggtgactcagggcgtccgcctgcttctcttgccaagtttttactacagctgggtagaaatgatagccatactgcctcactcaggctgtggagtcttcaaagaccacaaaagaaatctgcggacacatatatagacagtttgatcactctgttgcttgctttgttttgttttgttttgtcttatttaaagcaaaagaaaaaagacttaaaaataactcacagtttttagaagatgcaaatatttgttttatttttgttccaggtgtatttcagttttatttactttgactaggttgactttcctaatataccccgagaaggtcactattaggagaaggactgcccatgagcaaacttccttttctttttacaggaccaggaagcataaggaagaagcgatttgtgtccagcccccgttatgtggaaaccatgctcgtggctgaccagtccatggccgacttccacggcagcggtctaaagcattaccttctaaccctgttctcggtggcagccaggttttacaagcatcccagcattaggaattcaattagcctggtggtggtgaagatcttggtcatatatgaggagcagaagggaccagaagttacctccaatgcagctctcacccttcggaatttctgcaactggcagaaacaacacaacagccccagtgaccgggatccagagcactatgacactgcaattctgttcaccagacaggtaagacaggagcttatcaaccatttcatcaactcaactcggaggtcagccttgtgttggatgggatgagagggtgggggtgtggcggagaggaaacccagaaggggatgacatttgaaatgtaaacaaaataaccaattaaaaaaaaaaggcatctcatctgtattgcctcatttcctttcggttataggctagctcaatctgtcttgcttatttctattttaaacttccacatctcaagttctacagttctattttaaaagcattacagggaatcttgcttagagtcagtccttcaagcccagcaataatgaatggacaggcttcaaagtgcatgtgaagacacgcccaactgaagagctaagtatcactctctcctacttaaaagggatttcccttgcctctttgtaggatttatgtggctcccacacgtgtgacactctcgggatggcagatgttggaactgtatgtgaccccagcaggagctgctcagtcatagaagatgatggtttgcaagccgccttcaccacagcccacgaattgggtaagtcggcttcagagtacaagttaagcccaaatgcatggatacaacccaataagtcaatctgatgtgacgagagagaaaacatctcagactatgttgctacctcagccaccagcaattttagaaggggtagggtatattttccacgatttcaagtatggtcttactaggacaggagaaagtggtacaaacatttgaacgttgacatttttatacttgccctgatcaaagtgagtatgagccccaatacaggttgtctaataagagagccattgagcctcactcaataatacagctgaatgtccttcttgtctgcttcccaggccatgtgtttaacatgccgcacgatgatgctaagcactgtgccagcttgaatggtgtgactggcgattctcatctgatggcctcgatgctctccagcttagaccatagccagccctggtcaccttgcagtgcctacatggtcacgtccttcctagataatggacacggtaagatgacagctcctctttccagatggtgttcaaccttccttgtgtagggctctctctggctaagtgagctccatggctcttgctcatttcccctccttcagagttttctctggcaggatcataagtagtagatctttacctccattgcatcctgctcccaaagtccattcattcataaacaataacttctcgccattgtaaaatcagaagtcccctattgaggataacgtctcgataaaaatctaaagttccctagcattgattttcccaaaaatgcatgatttcaccaaacatgtattaataattgcctcttttttcttttccttttttttttttattattttaggggaatgtttgatggacaagccccagaatccaatcaagctcccttctgatcttcccggtaccttgtacgatgccaaccgccagtgtcagtttacattcggagaggaatccaagcactgccctgatgcagccagcacatgtactaccctgtggtgcactggcacctccggtggcttactggtgtgccaaacaaaacacttcccttgggcagatggcaccagctgtggagaagggaagtggtgtgtcagtggcaagtgcgtgaacaagacagacatgaagcattttgctgtgagttttcccaatgaaacatatccgtttgcaactcagggttgagaagggcaaagtgatggtttagttcctttcctagacaaactcctctacctgtgtcctgtagtgggactatgagatggtagcgtattttgagaattgattgtctgttttacatttttctctgattccctaaaatgtctttatagttctaacactgatatctgtatctccatttagactcctgttcatggaagctggggaccatggggaccgtggggagactgctcaagaacctgtggtggtggagttcaatacacaatgagagaatgtgacaacccagtcccaaagaacggagggaagtactgtgaaggcaaacgagtccgctacaggtcctgtaacatcgaggactgtccagacaataacggtgagtcatactggacttcagctctcagaaaccgggcaaaggcggcgtgccacaacatgtggttggaagttggaaactgggaacatcatcgccgtcgttctcttttcaggaaaaacgttcagagaggagcagtgcgaggcgcacaatgagttttccaaagcttcctttgggaatgagcccactgtagagtggacacccaagtacgccggcgtctcgccaaaggacaggtgcaagctcacctgtgaagccaaaggcattggctactttttcgtcttacagcccaaggtaggtgcttttacacttgaatctttgcaaaggagcctcagctgggcttgctgccatgccatacaaatgtttgggctgtctttacctattgatctgtgttccgttttgaatttggaatacttctaaatgcaggaacaactccttgctttgggatttgttgttgccttctgttgggaaggaagcttaaatctagctagcacttaaaagagtcttgcatgtgtttaatattgcttctctatccccaaagaatggccctttgaaaactcaagagccctctctgtataactaggtttcacatacaaaaattcatggttagataaattatatattaacatggcacccaggagttttagaaagtagtccaaagtacttgttactgggtacctagcagccgcacatacgagcacactaactaaggtaagagtttgagaattaaaaattcatcgttggaacatgtactttgaccaaagagactcgccatttcttttggtgttttgcagaaaggataaatcctgctttgaagaagaaaattgaatgaaatttgcttaagcttgtcatgtattcttagcattataagatagcaaactatatccaagttgtggatgaagtatttagcaagtgatttataaagtaccttcaactacagcatattattctaggtactgaccatggaacaataatcagtgtgacagtgaaccctgcttccattgacctaggccagcaaatatataaaatcaagacatttataagccttacagatagctatatgaactgttgaaaaagccaaaatgaaagtgaacatgtggcacgtgacaaggagactacttgtagcctgggaggagagcattcccagttgccatcacatcagatgtttaaccaccatggtgcatgttgtctccacaggttgtagatggcactccctgtagtccagactctacctctgtctgtgtgcaagggcagtgtgtgaaagctggctgtgatcgcatcatagactccaaaaagaagtttgataagtgtggcgtttgtggaggaaacggttccacatgcaagaagatgtcaggaatagtcactagtacaaggtgagtttcagaacgctcacttctgcagtagacacgctgtgttgctcagttggtccctagcatctacaagaccttgggttcaatccgcatgcatgtacctgtagtcccagtgtatgggagacagagacaagtgtgacaagacggtcagatgttcaggtcatctttgctacatagtgactttcagttcaccttggggaacatgaaaaacctgactggaaacacaaacacacacaaaacaattaacccaggtacttcatgtaatcccagtgttcagtaggctgacttgggaggatggttgctataaggcctaggttagcttggtctacataatgagttccagtataacctggcccacaagtgaaccctaaagttaattaatcgacacatgaaacaaaacacatgctttggagaccctgtaattttgatatacgattttgtaggactaaggaaaagtcacatttaaaagaattgcctatttttaaagcaatgtgattgattaactcattgaaagacatatacctgttttctttgtccacagacctgggtatcatgacattgtcacaattcctgctggagccaccaacattgaagtgaaacatcggaatcaaagggggtccagaaacaatggcagctttctggctattagagccgctgatggtacctatattctgaatggaaacttcactctgtccacactagagcaagacctcacctacaaaggtactgtcttaaggtacagtggttcctcggctgcgctggagagaatccgcagctttagtccactcaaagaacccttaaccatccaggttcttatggtaggccatgctctccgacccaaaattaaattcacctactttatgaagaagaagacagagtcattcaacgccattcccacattttctgagtgggtgattgaagagtggggggagtgctccaagacatgcggctcaggttggcagagaagagtagtgcagtgcagagacattaatggacaccctgcttccgaatgtgcaaaggaagtgaagccagccagtaccagaccttgtgcagaccttccttgcccacactggcaggtgggggattggtcaccatgttccaaaacttgcgggaagggttacaagaagagaaccttgaaatgtgtgtcccacgatgggggcgtgttatcaaatgagagctgtgatcctttgaagaagccaaagcattacattgacttttgcacactgacacagtgcagttaagaggcgttagaggacaaggtagcgtggggaggggctgatacactgagtgctggagggatccagtgagtcaaaccagtaagcagtgaggtgtggcaaggaggtgtgtgtaggggatacatagcaaaggaggtagatcaggacactaccctgccagttacattctgataaggtagttaatgaggcacagtagcatctgaaagaccatacagagcactaaggagccccaaagcactattagtatctcttttcttatatctatcgcccaaataattttcagagtctggcagaagccctgttgcactgtactgactagatacttcttatcacaaagattgggaaaggcaaagcagaaagatggtaagactgggtttcaaacaaggcttggtttctatcactggaggcaaggaggaggggacaaacaagatcattattcgaagtcgctggttgctgtggttttacggaaggttgatgcatcattcctatcaacagtgaaaagttcagcttgttcaacgtgacagaaaggctcatctccgtgaaagagctcctgatttcttcttacaccatctcagttcttaactataattcatgttgaggtagaaacaattcatctatttataaaatgtacattggaaaaaaaaaagtgaagtttatgaggtacacataaaaactgaaggaaacaatgagcaacatgcctcctgctttgcttcctcctgaggtaaacctgcctggggattgaggttgtttaagattatccatggctcacaagaggcagtaaaataatacatgttgtgccagagttagaatggggtatagagatcagggtcccatgagatggggaacatggtgatcactcatctcacatgggaggctgctgcagggtagcaggtccactcctggcagctggtccaacagtcgtatcctggtgaatgtctgttcagctcttctactgagagagaatatgactgtttccatatgtatatgtatatagtaaaatatgttactatgaattgcatgtactttataagtattggtgtgtctgttccttctaagaaggactatagtttataataaatgcctataataacatatttatttttatacatttatttctaatgataaaacctttaagttatatcgcttttgtaaaagtgcatataaaaatagagtatttatacaatatatgttaactagaaataataaaagaacacttttgaatgtgtatgcctattttctggagtgggattaacttctgggcaagaaatctgatgagacacaaacattggacttcaagacagttttaaaatttgggtaaatgaactgtatttcctgtttatagacgtactaataaaaaagaagttgatgatgtctttagtggtaagattgttactaatgtggttggcaaattgctgtaaagagccagatagtaagcatttatggcattgtaggctatctttcctgccacaaccatgtgacagtgagtgctttgtaggactgagagcagccataaatgacatgtaaatgataaactgtggctgtgctttaataaaactttatttacaaaa

SEQ ID NO: 22 - DNA, 5722 bp, Unknown
  Description of Unknown Organism:Unknown
ggacgcacaggcattccccgcgcccctccagccctcgccgccctcgccaccgctcccggccgccgcgctccggtacacacaggatccctgctgggcaccaacagctccaccatggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaaaagatttaaacaggatggtggctggagccactggtccccgtggtcatcttgttctgtgacatgtggtgatggtgtgatcacaaggatccggctctgcaactctcccagcccccagatgaatgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccgcaccccagtttggaggcaaggactgcgttggtgatgtaacagaaaaccagatctgcaacaagcaggactgtccaattgatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcatcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcaccttgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttccgtaattactaggtagttttctaattctctcttttggaagtatgatttttttaaagtctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattccttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagaatattcagattgtgtagatatgctatttaaataatttatcaggaaatactgcctgtagagttagtatttctatttttatataatgtttgcacactgaattgaagaattgttggttttttcttttttttgttttttttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatattt

SEQ ID NO: 23 - DNA, 42521 bp, Unknown
  Description of Unknown Organism:Unknown
gatcgttttccagacatttttgttctctgttcatttccttatcgtattcaaaaagttatcacaaatgaccttctctatctgtctgcgtctcttttaactctcaccgtttgggacctttcaaatagtttttcgctatcaaatctaaacattagttgcgttgactcgacatttgacccctcactatcatctccagttctcttttttgttacactttagcagtggcagcagagagcaagtaggtggagccaaagtgtgcgccattcatcgggaaaaattgtgttcttcatcaaattttgggcaatttactcgggatttgcgctaatttggaaacaaaaattcaaattcctgccaattgttttgtttgctttttttcttttttttttgctcctcccatctctcatcaaattgctcttttttcgattctaacatatcagccatcttcagagtgtgtcactaacccccatttttattcaaggttagtgatatagtatcctaactacagacgtcacaccatgaggttgctgctcttctcggcagcccttcttctgtgctccgtcccaacgtgggccttctctctgtcatcattcttcggaagcgatgttgcacaagtaagcaagctctcctatacctagaatcttgtaaattgaaaactctaatttccagaagccataccttcatccaaactccccaccggagcgtgacccggcgagttccagaatgaagagacaggcatatcaagtgtacgttgatggagatgtttccgttactgttgacaagtctggacaaaaggaaaccggcaactggggaccatgggtgcccgagaacgagtgctcacgttcgtgtggtggaggagttcaactcgagaagagacagtgcaggttcgtggacttttcattttttagggaatttcctagacgttctaaaagcttattttcaaaaattttggtttcctgatcttcatgcctttatgaacgtggtgaaagatcaacctaggctagcctgtgacatacattttttgaagcagatccaactttatcaagagccatcgaattctcgttttaaagtgttttttttttctgataacttttttctaatagctttacccatttttatgtcaagactgaaagcaatgaatcacaagaggctatctacgtttgtttttgaagctctgtaggaatcatcttaaaaaattaagtaaagtaatggagatgaaattctaattttttaaaatcataatcattactttctgtattatcttcaagttcaaacttttcaaacggttattctcaagaaactcacatagaattttaacaatttcctctatctatttcttgcaagcaacccaccgaactcaaatcttatccaaactaaacttttagtggtgactgcactggagcttcagtccgctacatctcgtgtaacttgaacgcatgcgagtctggtactgatttccgtgctgagcaatgctccaaattcaacgatgaggctcttgatggaaactaccacaagtggactccatacaagggaaagaacaagtaagttaactttcttcaagatgtttttctaattttcgagttttcaggtgcgagctcgtctgtaagccagaatctggaaacttctactacaagtgggctgataaggttgttgatggaaccaagtgcgactccaagagcaacgatatctgtgttgatggggaatgtcttccagttggatgtgacggaaagcttggatcttgtaagtttaaaatttaattcaaaatcttcatttcatgccgaatatttcagctctcaaattcgacaagtgcggaaagtgcgatggagatggttctacctgcaagactattgaaggacgtttcgatgagcgcaatctctctccaggataccatgatattatcaaacttccagaaggagccaccaacattaagattcaggaagccagaaagagcaccaacaacttggctctgaagaacggttccgatcacttttatttgaatggaaatggattgatccaagttgagaaggaggttgaagtcggaggaactatcttcgtttacgatgacgctgaaccagaaactctcagtgctcaaggaccactctccgaggagctcaccgttgctcttctcttcagaaagggaagccgtgatactgctatcaagtacgagttctctattccacttgaggaggaagttgactacatgtacaagtttgacaactggactccgtgctctgtatcatgcggaaagggtgttcaaacccgtaatctctactgtattgatggaaagaacaagggacgcgttgaggatgatctctgcgaggagaacaatgccacaaagccagagttcgaaaagagctgtgaaactgttgactgtgaagccgaatggttcactggagactgggaatcttgctcatccacctgcggagatcaaggacagcaataccgtgtcgtctactgccatcaagtattcgctaacggacgtcgtgttaccgttgaggatggaaactgcaccgttgagagaccaccagtaaagcagacttgcaatcggtaagttgattttataaatgcataaacaactctgtgaatctatttgtttatgcgatgctatccatatatattaccagatggtgttggtgcccaaaacttataaacaattattttctctttgcagttttgcctgcccagagtggcaagctggtccgtggtcggcttgctcagagaagtgtggagacgccttccaatacagatcggtgacctgccgcagtgagaaggaaggagaagagggaaaactcttggccgctgatgcttgcccagctgatgagcaagagaagttcgacacagagagaacttgcaatttgggaccatgcgagggacttacatttgtcactggagaatggaacttggttagattttgcaaaatatggggacctggggaaaagcatactaaataagatcaactttatgaaacaaataatttttagtgcacccgctgcaacgatactgaggagactcgtgaagtcacctgcaaggactcccaaggaagagcctatccactcgagaagtgtttggttgataactccaccgagattccaactgatactaggtgagtcattccagatatgacattgaacttggattaatttttttcttccagatcatgcgccacccaaccaccatgtgagtacgagtggaccgtcagtgagtggagcaagtgtaccaccgaatgcggacacggacacaagactcgtcgtgttatctgtgccatccaccaaaacggaggactcgaggttgttgatgaaggacactgtcaagctgagaagccagaaggaaagactaactgcaccaatgaggagaagtgtactggaacatggtacacatcttcatggtccgagtgtaccgctgaatgtggtggtggatcccaagatcgtgtcgctgtttgcttgaactacgataagaagccagttccagaatggtgcgacgaagccgtcaagccatctgagaaacaagattgtaacgttgatgactgcccaacttgcgttgactctgagttcggatgctgcccagataactctacttttgctaccggagaattcaacttcggatgctctaactgctcggaaacagaattcggatgctgtgctgacaatgttaccgttgccactggacctaactccaagggatgcgaagaattcgttgagtctccacttaaccttgaagctgatgttgccaatgctgacgctgaagcttcaggagatgctccagaactctgcagcgtcacaaacgagaacggagaagctgttgatgttgagtgtgccaccattgctccaatcactgctcttcttggagatggggaacttatcggaaatgatactgatgcttccaatgagaccatacactgctcgaagaccgaattcggatgctgtccagattggtacaccgccgcctctggaaagggtaacgaaggatgcccatcgttcactcttggaggatgtaacgagactcaattcggatgttgtcacgatgatgtcactcttgctcgtggagccaaccttgaaggatgcggagagccatcttgcgctgcttccctctatggatgctgtaaagatcgtaagacaattgccttcggaccacactattctggatgtgagcgatcatccttcccatgtgagcttagcgacttcggatgctgcccagatggtgagactgctgctcttggaaagaatggaaccggatgcggagagaactgcttgaccaccaagttcggatgctgccctgatggaaagaccaccgccaaggggtcccacaacgagggatgcggatgcgagttcgcccaatacggatgctgcccagacggaaaatcagttgccaagggagccggattttacggatgcccagaaagctgcgcccagagccagttcggatgctgcccagacggaaagactcgtgctcgcggagagaacaaggaaggatgtccatgccagtacacccgttacggatgctgcccagatggggagactactgctcttggaccacgcaatgatggatgtgataactgccgctacgccaagcacggatgttgcccagatggagagaccaaggctcttggaccagatggagccggatgcccaccaactaccacgccaccattcctcatgggaggaactgttgccccacataaaatcgccgcctgtaatcagacacaagaaagtggaaccgtctgcggagccggatacaagcttgtaagtaattaacctcatgaaaaagaattggagcaacacatttcatgtataaatatttcaatttcaggcatggcattatgataccactgagggacgttgcaaccagttctggtacggaggatgcggtggaaatgacaacaactttgctagccaggatatgtgcgagactatctgcgtcgaaccaccaggcaagggaagatgttacctgccacgtgttgatggaccactccggtgtgaccaacttcagccaagatactattatgatcattccaagaagcactgtgtggccttctggtggagaggatgtctcggaaatgccaacaacttcaactctttcgaagaatgctccatgttctgtaaggacgttggaccgtacgatgctccaaccaccgctgctccaccaccaccaccacagcaaaatgctcagcaataccttccaactccagaagttcaacagattgagattcaatctgctgagcaacctcaaccacaacagccacaacaacagcaacagcaacaacagcaacaaccacagcaaccacgtcaatcaatggaagacatctgcagatcccgccaagacgccggaccatgcgagacttactccgatcaatggttctacaacgctttcagccaagaatgcgaaaccttcacttatggaggatgtggaggaaatctcaatcgtttccgcagcaaggatgaatgcgagcagcgttgtttcttcgttcacggagctcagccatccgctgcccggcaggaacaagctcagccagcagctcaaccagctcaaccagctcagccaagtaacatcgtctctccaccacaacagtcagctagtccagttgtggttccatgtaagttctttagaatgcatttatttcttactataagtttctataagttcgcatgtgaagcatccccatttcagcgaacagcaaacaacgcgatgcttgccacctcaacgttgaccaaggacgttgtaagggggcttttgactcctggtactacgaagttgccaccggatcctgcgtcacattcaagtacaccggatgcggaggaaacgccaacagatttgctagcaaggatcagtgcgagtcactctgtgtgaagccagcttctgaagctgcttcagccggaattggtatgctttgagttatagagaatgttcactatttttgttaaatgtttgagtaaatgagaaactggctcagtttgaaaatgtttgcaccatgtttcaaaatagtttttgagttgaatagttgaggccatgaaaatcttaattacactccagaagtacattttaaaacatttttgagaattaggtcttcaaaaaaaggtttaatattgaggtttcaaattagaaatattaatatacggggatttgggtttaaaactgatttttaaaatcttatttttgaagtttcgctttgatattcgtgcaaaaaaaaaaccaactttttcagacggtgcagctggaatcaactcagtttgtgacgaagccaaggacaccggaccgtgcaccaactttgtcacgaaatggtactacaacaaagccgacggaacctgcaaccgattccattacggtggatgccaaggaaccaacaatcgattcgacaacgagcaacagtgcaaggctgcttgtcaaaatcataaggatgcttgtcaacttccaaaggttcaaggaccatgctctggaaagcattcctattattactacaacactgccagtcatcaatgcgagacgttcacttatggtggctgcctcggaaatactaacagattcgctaccattgaggagtgtcaagcgagatgcccgagtaagttctaagttaatagtgatatatgctttgtttcccctttattctttgacaattttcaaatactttttgcataattaccttatttctattcccttctgtttcccattttcctccacccgctacaaattgtttcccgtactctctcctttctcactttcccgtccgaagggacacggcaatgctgcctaaatgaactgcctaataatatttatgaattttccaattttctaaaaaaaaacaattctctcaaaaaattccctgccgttccgccactgctttcttcacccattgttgcgctattttttttaaataaatgaataaagctgaaatagttaacagtttctgaaattgcatgtaagtttgtagtgtatcagtgtgtttgtcgtgaaagtttttttttacctgcatgatttcctgaactgcatgaaactgttcttattacgttttagatttgctgaagtgtgctagaagtgtgattttgtttcagaagacgaccagactacaacaacatcacaaccagaagagctcccaagtttgccacttgttcaagaagatcctcagccacgaccggcattttcattgaagtaagcacgtgtagtccaagtgcctacttctcgtatgaccaaaaaatttaatataaggtttccaagtattaaggaatcagtagcatgtaaattgtgtggattgttctcctgggttgatgggtttttttctcactcacaatcagatatggagtagcttatatgggaatttatttgagaaatagaatatgtcataacatccaaatttaattattaaaaagttgtgaagtttctcattatgtatataaaattcgcctttcaaataagaacaaaaattaactgtatgaaagagctgaattcaatttgaaattgagaaaataactggttcaaaaagaagaaaaacgttggaaaatctagacgtaaatctatggattttcttttcaggtcggggaaatttcgacgatttttatattttcaaaaatcattcacaaatatacaccaaaaattatttttaccataataaaatacggaatttcactggattactgtagtattcatgtaaggttactgtattgttactctagggatactacaagaatatttttgcaaagttgtaagaagtatagagattactgtagattgaaaatctagacaaaaatcattttccgtaataatctgtggggatagaatgttgaaggcacaaggcttataaagcaccatgggaaaaaattttaacagtgatttttttaagcatatcctctttcccaggaaatccacttttcaaatatattcccactaaactctttaagacaatccttcgcccatagtcgtcgccgtgatgctccatttgcacgttccgtatccgcccgtcaccatactcctgattccgaagaggaacgagttgactgttatgctgttccagatccaggatcttgcgggtaataaatctcacctatccattacaaccattaccgtcttaatgattcagagactaccgtcttgtttggcactactctgccacgagtaactcatgccgtcaattctactatggtggatgtgctgggaatacgaatcgcttcgagacccgggataaatgtgaaacatcgtgtgttgctaagattgaagaacgcgtggaaagtgtgtcagaagcttcaaaatctctggaagaggttagactaacggatccaaggatggattctcactttggatatcatgatccagaagttgatcaaatcgaagaagaagctgaatatgtcattgttgataccggagctctacctgaattatgcatgcttccagaacaaagagggtcttgttatgataacattttgagatggaggtaagtcaaatcaagaatagaaaattcgaaaatccgaaaaactttataattatactaaaagcaaaatcttaaaatctttcagattcgactctgaaaagtctcaatgtgtaaccttcatgtattctggatgtaatccaaatgcaaatcacttcactagtcaggttagtttcattattttgtgtcctttcgtggaactggccccttggtttctaacttgatcttctccttccgaatacccaatttgagcaccgctggctcactttttcgacggtgacgttcctcaattctagcggcctctgtattttctgagcactcttgagcaacagtttcctcactggaaatgtttgtttttcaagagggagtgagagagagaaataaacgtacaatttttgaagccgcacatgatttgttagaagtcgatgccgttctgcagtatccttcatgtttcgtagttgtttctgtagtaatttttatggattaggaactaagaaatcatcactcactgcggtagttgcatttttgtgcatgcatcttcccataaaagcaacaaatgcaacaactgatagagccgccacacaaattgcaataattcgaagtcgatttctaattcctttctttactttttgtctatgctcagctgctttttcgatgtgcttcttcttgctggggtcgagctcgcaatgaggaaatggttcgatgagtggaccgtgttttttgcattgttcacaacggcgtccagtgtatttgtctgggcagtcacacgaaagagtgcggtttttgaaatctgaaaattttaaatttaagaacaggatctatagcagttttgcccatcacagtcctatgtctatattaaaaaaaattatcggacattaaaaaaaatgttttctcattttttcagtatttctataaaaactgcattcgcatttaatcataacttttaatcgttaaaaacttagtctttaagtacctggggatccgtaaacacagacaatttcatcacaataatcgccttcaaatcccacatcacagatgcatcttccatttctcaaaaacccctcgacacatttacttgtatattggcattcacttccaaaatatgagcccacacattcacatcggtcccccttccattctcctttgtttccgcactgaaataattcaatagattttggaagtttagggcctcaaaaatataccttttccgctggccgatagtcacacatttcacctttccatccgacttcgcaaatgcacggctcactgaatagaaggctttccgggtcgaagcggaatccgaccgagagtccgtgaactgtaaattgaaaatttgtaattccaaaaaaaaaacagcttttgcaaaaatcgtccaaaagaattttagagttagacattatttttctcaaaaagttcaaagttgtatcagttttaaaataaaatatttaataggattgtagagcttgttagaaaaaataaaagctacttgaaaaaagaaaggtatccaaaaaggtattgagatagtttcaagcaactctatttgtaaactgtcgagtttttaagttctacaaatctcttataacatcgctacatctactatcaaactttgaaaaaaaaccataccacattcaaaatgttcacatttatctccagtctgtcccttgatacaatgacaaatccctccagcatagattcctccattacgacattcggctctcggatcatccagagcaacattgtctagaatacttctcttttgaagaatacgatgcacgtcgctcaatatattttcatctagatctagtgagtcatctcgtgattgtgcttttgttgttgataaaaataggaagagtaaagtggaaaattgtaaacagtacatagcgttagatactgacaagtctactatcaattgatttatttattgcgtcttgaaaggggtatcaatgagagaaatagggagatgggtaaaatgcatttataagagaatacaaaagatgacgtaattgattaatcagagatcagttgaaaatacttttaagtatcaattattatctgtgaagacagtcacgtgactctgactcgaactcaatttgcatgttgatagttccaatgttaaagaaagtctttgggttttctccagatgaaacaaatgattttggaatattaaacgtgactcttctctgacaaggtttgagtccgtcatcacaatcgtgatagatattaagttttggatcaatagtcatcacttcggaagtgtgtccggtaagaaggaattgaccaagagagtctgtagttccttcggcaagaagatcgtcaagatccggtcctgaaaaaaacttttattttgaaaaatttcaatgagttgcttcatgttagaatttggaatttttaaagatgttagcaattggtatttaaatgttcaagctaacgtaattagagttattcaaacaagctttatataaaaactttgtgtaagattcggtctaattagaacatcaatttttaacgcagctgataaaaaactttaatttcaagcttcacataattctacttaccggtatcatcatcgtagagcttcaccttcgtgttagccagtggtttgtctccacacatcagaacacccttaactccagctgattgggtgaatacagcttcggagccaattgcacaaagtatgaaaagtgatgaaatgcacgcgagtcgtgacattatttttgtctgaaaatacaaacactgactgatctgaccttcatcggagaaactctcttatagcacagttggttagaaaaagatacggagaggagaagtgggaaatcgaattgaccaaacaaaagaactggttttcacttgaaatagaagacgatgaaagatatacaacagagaagatcggaagtgattcatctggagaagaaaattgagaggagcaacttcttgtattttccacttatttatatacccaatagaattcacctgattctttccgatttgtgtacatttcgctgactaacgtgtgcttcttcggttttgtcatttcttattgttcattgaaaataaacagaacaaagcaatcataaggtcgaaaatcccatttagagatcaagaggtgtacctttaattgtgcggcatggcatagttttatcttgctgaactctcaccaattgatgagtatgtcagtagaatggattccatccgatcgttgctccacggtgatctcttccgccgccttttcatccaccatacccgttgtgtatggctggcaactgtgaacagcgcctcagtggaatgtttagtttgatatacagtttaaaataattttctaaactaaagaaatcagtttttgaaaccagtcttgtaggcatgtcgggcgcaggcacgctaacgtgaaaaatagaatttcgagtggttaactattttattttcaattaaaatacaatcaactacacaatgaatgacccggataaatgaaatacaaatacaagaatttaaaaaaaacatggaaatttaaacttttccatcatctccctttgctggaatattatatttcattcgataagcttccaattcggcttttctctgatcggatcgtacactgtgtctctcatccatctcttgttgagctgtcattctcttctcattccatttctgagcttttgcttttttgtggattctgttgtattctttgcacttgcagcagcaataacagaagcaggcaatgagaattacagcaatgattccagcgcagatggcaataacgattgcggcggctgatgtgctcacccagcagacattgtatttgacatgcttgatattacagtctggatagtaccagtcgaatggcatacatcttttcgtttttccaccacaccagaagcaattctgaaaaaatgtgtttttgaaattttcaatatgtttgcttataaaattgaatttaatttttcaaacagtgtttcagaaactcaacttctgaaattaggaaagtattctcaattgagagctgtttttgtattaaaagtttcagtttagaactacaggtgtgaaaaaatctgagcaagtgaacaccaacgtattgcatcacagtttacgcgtcaatttattcgagtgttcattgtagagaaagttaggtcaccttccagaaaattaagaaacttgtttcagacatttttgctcttttagaggaatttttttttagaggaaacacgcaagtttctttgaaaacaaaaacaaaatatattttttatccacttaccgagcccttgccaacacatgtttcacaagtgttcaaatcgttcgatccaattctacagtattcttgtttctctgaccatgtcatgttatccgcacatactgatactagaacaattgagaaaaagagtagtaatcggtgaatcatcgttctgaaaaatcaataaatagtaacaacttgagcaagtctcgtaactgagcgacaaaaccaaagtagtaatgaaatagaaagatagaaaggtaaactcaaagggctcgcgtgtgtttgtctatcgagtgccaatgagttttaggagtagcgacagaaataagttggcagaagaagaacatacgaactatgtcgggctacaagattcttgtgtttactttttgaaaaagaaaatgcatttgagaaaatgcaaatgttcggcagaaatcgaatggagtttagagcagaatggtaaaaataaaggtggatcagcaaaaatagttgaacaaatattttgtagatttcatgaaagataacaaaaaaaaataaatacagaaaacaatatatgacgtatttttcaatcattgtttttgtatagtgcaaattcagtagttgtacctgttataagtacagcgaagttatacattttagagtgggtcttgtcacgatccatattttttgaacgcaatatttgaaatccaaaaaaaaataaagaaactaggcgccaagaagctatagtagctatacgcataaattgtgaataccttgaattacattaaattccaacaaaataggaaaatcatataaaaacgaagttagttgtcaattcaaaaacgtttttaaaattgttcataagcgccgagctgtccccctcagttttcgtttattcagcttttctctctctctctattctctatcgtcacctatatttcatagtccccttatccaaaagtggaagtgaatgaggatggaaatatgataccgcatgcttcaaaaaaatttgcttatgagaaaccaacatttgaaaatttccaggaaacttgtgaacgagcctgtggtaaatggagaaatgtggcagtgtgcgagttgccggccgaacacggagattgccaacttgcgattcccaggtatgtactgttgacacattttacaaatgggatgggaagtggtcggtgatcaggtggaaatgttgatggcaaggttttaaatagatgtagtaactgaaaacaaaatgacagatgtacatacataaattaggattaaaacaaaaatactatgcggagtcaggtgactaatttttctggaaattccagaatttgaaaatgtttttctctgtttgaaagtagaacgggaccttttacaaaataggctgaggtaggtaggctgtagaaagtgcctttggtgtctttgtaatttttgttttcaaaaaatcacttgtaagcacatgaaaatcacatgaataatgatgtaaaatttagaaaattagtataaagaagatttacattttaataataataattccagatggtaccatgacccaaaaacatcccaatgtcaaatgatgatgtggactggatgcggaggaaatggaaacgcgttctcttcaaaagcagactgtgaatctctttgccgagttgagacattatggtccaacaacactgacttctgtacattggaacgatcggccggtccatgtacagattctatttcaatgtggtatttcgattcaactcatctcgattgtaagccattcacttatggaggttgccgtggaaatcagaatcgattcgttagcaaagagcaatgtcagcagagctgccgtcctggagacacaaaatctgaggatatctgcacactccgcccagagccgggaccgtgtcggctgggactcgagaaatacttttacgacccggtgatccaatcctgtcatatgttccattatggaggttgtgagggaaatgcaaaccggttcgattcagagttggactgcttccgacgatgctcgagtgtcaaggttgaagcaagtgaaagcgagagagtgggacagctgacgtctgcatccacgccagttatttatattgttaacaaaacagcgatttttgttggaaatactgtaagttattaattttaattcgaagatttcttaatatttaaactggtcccatgagagtttggttcattttccgacaatagactgcaaaattgataacttttcatgaacactttagccgattttagctagttttgtttattaaaatttggtaattcaaaataaaaaccttacgccactccacttttgaatacttgtcaaatacattttttcagttccgaatccgatgcaacagttacggagtgcttccaataacatggtacaagaacggaggtctcctccagttcggctcgcgaatcactgaagagaatgatgacactttggaaattgtggatgctttaactgctgacgccggtgtctacacttgcattgccggccaggatagtacaatgagcgagggagtcgaggttgtgatcaagagacttcctggtcacagaactacatctcgtccaatgctgacaccatccaagaacttctccttgggaaccccaccgacaccatctccatctacagtttctacaacacccttccgaatctatacgcctggatctgctccatctgatgctcgtgtaagccgcccgacaagcaattcctgtatggatgtgggtaacgcgagcacgtgcgatttgatcgtgaagaacggtttgtgcgggaagaagcgatatggaacattctgctgtcacacttgcacccgggttcataattttaaattttaagtttggattttttgatttcaaattttcattaatcttttaatgttttctccttcataatatctccattgcgagatctctttttcccttctcttcctatactttcccctcagacaattggctaattactcgttcgttccagtaaataaatatgaatttatttcttcttcctatactttggtatacataatcatggcatgaaatacaagacaaaaaaaacaagaaaaaacaatccacttgaaatccattcaggtgtgaactaacatcttactctattaacttcgtgccattacttccacttattttgcctattcactaatgaagtctctgagaattattttctgtctaactctgctgattgcaagcttcccagctcagcggagccgccgaaaacagaaatttgtacgccttcctagtgggttcacgtttcctgcggatgcggcgagtaattttcaaagagatgcgtatattccagcgacggtaaattttcgctttttgttaaatgaatttcaggcttcaaattattttctaggacaaaaatttaaagtaggcttgcgcatactcatttccctgccttacctgccaacaggctagcttttggagagaaatcaaaagtttggtgtctgtaaatctaagctttccgaagcgtccgaaagtttttgggaatccgctatacactttaagattgataaatatttgaatcaggtttattttgcactattaaggcgtgtaggcactaggccggcaaagctcgcctacggggagccttacaatcaagtattattcatgaaggtcttgatttggttacagaattccatctaaaattacttatacaaaaacatgaaaaatttcagtttgccccgccatctgagaagattcttcaagctccaccacgctatttaactggagaacacaatccagcttatggtaggcccaattttttatctgattttctaaatttaacttcaagctcacaataccgatgtgcaaggaatgaactacgctgagtacaagcaagcgatggccccacaaccacatccagtcgatgcttattctccaccacctcctgcaccaatggtcccaccggttactgtagttgaaccacctgcaatgccgtatgaaatgactacgattgcatctgttggaccacttactactcccgcatcagtcggcttgaagaagggaaagtttgtgattttagttaattgatctttcaagtaattggatacaatttccagcatcggaggaattgctcaaaacttgaacgacaggtacaccagcttaacaccagaagctcaacgtgctcagaaaggtcatacctatacggctctgggcggtggacaattctatcaaagtttacttggaggggtaagatgcaaggttagaacttacaaactcaattcattttacagaaaggaggccccggaggattctccccactctcgttctttctaaacggcggtctaggaggtactggtggtggtggtaacaatggattcttcgttccggtgcctgtagtcattccgcctccaccgccaccgccaccaggaccaaactgtttcacgaacccgtcgggattcctttgctgtaacgtgacacttgagaaaactatggaagacgcgtacctggccgcaaaagcagatggtgcatcactgtgcaatgtacagaaaatggcaactgcagtgcaagcggtggggtttatggatttcattttataatgtaatgtgctcttccctagaattgaataagcttacaacttgaattacgacttgaattacaacttgaataagcttaaaatatccaccaaatttcagcaagccgaaaaaaaattcggaacaactttcgaatcagtcgctgctcattcggacttcgtcgcaaaaattaattttgccggtgacctgaactgtaaaatagaaatcgatgggaaattcatactagcgtacgcaactccaatcgccgagcaagaggtgaacattgtcgatgctagctcattcttctcgggagctgctgataaggatttggatggtgtcaatggtaccaagcccacctacattgtctacggtcccattaaataatggagggtctagctttaaagatttctgtatattaaagctgaaatgtgaattaattgtttatttgccaatcacaataaagttggaaatatcatttgaatagttcgaaagttttcaatcggaatgggagaaaattcgaaaatttaggtggaggtgaaaagttgatgaagtaacacaattaactgtgctcgaatcctgaatagaaggagaaaagagcctataaacagattttcaatttacacatattacacaacaattcaggaagaagacagtagttgcaaaagaaaatacgtagaaaaaagagtgaaggactggcgggatgtcagtttggatgtacaaatagaactcctgaagcataagaaacagaagaatcgaccgatgatcgaacctgaaatggatttattgttgattgaaaaatattaagcaattctgaatctctaccttgtttgattgtgtgtaatgcaagaatctaaactcgtgagtgtgattgttactgatccggaaatgttcggctgcttgcagcattatcaatatcggattacgcccacaaatcgtgttctgggtctttttgaggtagtcattaaaagctgccggattaagcgtctcaattgcgctcattccctgcttatccatattggttatctgctcataaatcggaatagaactatgacgatcgtacggagaaaagctgaagcgttctccccaatggcaaaagtccgaagagatcacaaacaagtttcttggatcctccatgtaatgagcaaaaatatttccatacgtttgctgcctagatcctggtaaagatccaacaagtaccggaacaatggtgtaacgttttgaacccataacctttgcaataaatgggagttgcatttcaatactatgctctgattcttcatctcggcgatccatcaaatcgaaatgacgagtggcacgaagctcctcgttaactgcaaagggcaatgttgtaaaagatgtactaagagtgcaatagattacttttgtgatcaacgatcaagtcgccgagtggagttctgtacttgctgcatgtggttatagcacatccatttagagcaacaacgtgagatgggccaagaatgaagactctttcactgaaagttattgagtaagccctgttgccaagtacaaatttcaacaactcacactgctgatgaaacaacttgtttgaaagcatatgcagctgtttctccacaatacgaatatcccgcatgtctgaaagttatcagaaaataaatattaaatgcatttagagtattacggtgaaatcaacgctcgagccgttccaatccgtggaccggcgttgtcaagccattttgtgagttgccgatcaagatctcgctggttggcgttgtaccatgatccggcatgtgaggcagatctcgtgtgctcgccgaatccgtttagtgacattttaaattcagatggtctgaatattaaagttttgataaattgttgtatacgacttgattaatatgtttagtagggttttcaactactgtgtgtttcccaaatagtcaacattgaaaaatggaaaagtttgaatttaaatattcaaataattttaattaattaatattaaaattcacaatacagtgtaacatcacacttaattcaagatgttctaaaaatatgagccatcgggctagctctacttcacgaattcgaatcaagtccggggaactggctcgaaagaaaataaatttttaatttggtttatgtccgaaatagaaatgggaatctggtttttcattctgaataatttccgagaaacacttacaaaataaaattcagatatcttgcaaaaggaaggccaaatgtcctgagaaatagagcacgagagttttgaaatacctgcaacaacaggatttgcttctattttgttttttgaactgaattttaaactattatctattctgaaaacattttttgtccaaaaaaaatcaagaacaatttagagcaaaatgtggcaatccgaaaatgttgatgcaacaaaaaagtgttttttttttcattgaatttcagttttgaaaactgatttctttccaaaaaaaaaacgaaggaaaattttgagaaaaaagtgaaaatccaaaaatgctgattttggtttttttttcaaaaaaaaagcattttgcaaagtgtgtgctttttttcgaaagtttcagaaccttgagacaaaaaaccaaaattgtgttcccgagtgaagcccgccacgtggacatggtcagacgaatcttgttcgtgttcgcagccaattttcatttttgctgaacgcataattgttcaaagaagattcggtctaaaaagacgaaattgaaatagattgtggaatcctttgaaattttcttttgacaaaaggtcaccgttattcaaaaattgagatggtctcgtgactaaaattaaacaatcaagataatcatgattgtgggcctgttttaaaatacacttttcaaaaacgaaatgtaggctccaatccaaactgcgcatcaagaccaagaatataaaatttttaaactcgggagacgtagagaaactttgaatattaaacatcgccgtcaagtttccgtcagagcgcgcctgaaattttttagaggcttctttcaaaaagctacccatacaaataatcataagaaaaacgttttaaaactttgcattccacccaaaaatgtctgaaattacccgtaaaaagaatgtgtgaagggagtgatttgagggttctgtcaaacagtttgactgtttcgcgttcgacgtgtctcgacgtggatggtattgaagaggaccgcgctgatcttgtgctggtcgtcgtcgtcttgtcggaccgccgcgagtagtcttcagtctaccaattacctgaaaatttgacactttttgtgatgtgaaactggctgcctgaagcaatgccatataataatcataataataataatgaagagggatgaggatgcatgccaaaagaatgaaaggaaagacgctcttctacaacaccagccgatagtatttagaagaaaaagaagactaaaaagagagtattgggtgatgggagaaagaacacaataggggaggcagtgaaatagaacgagaacaatggaatcggcagacatttgacactagaggggccactgtttcagtctttttcgcacttgaatattggaagagggccaagaaggggagttccaagaatggaaaaagtggtaggtttgtagaaaatctgcctttttttttttaaaatttcgtgttcactactttatttcgtgttcactcgtttatgtcttccattataggcaggcaaagtttcatgcctacatacctgcctcatgcctatttgactttcaatataaaacttgatttttggcattcttcattttataacaattgtaactaataataagctttgcaaagttttctgaaagaaattgtctaaattttcctggtacactgaacatttttcggtataaaatctatgcgtatcaagcctatttctaagagccgtaagtattttcagctgaaaatgtaaaccacggagtcaatatttacttcgtatcatccatcttccattccgtcttgtttacacctacggcaggtatttagacacgaatgattgtttttctcgttgcctaatactttttcccccgaaatattcccatattccagttctgaacaatgcacttttcagcggtcatcgggtccatccagccctcattcagccctttcatttatcttcgtttctacttttagacgaaaatgcaaaaaaaagagaaaaagacactctcttttgacgctcacattcgctcacattgctgtggtagaaaaacactcactcggtggctgctgggaagggaaaacgagaaaatgtttggtcacgcaatacgcctatatctttgatttgactttgaatctttatacatttttcacggggttcaaaaacaattatgaagaaaattgtttgattaaattagaatgtagattctttatattttcaatcaaaaattaattttggaaaaataactatccaaaaaacgaaaaaagtaataaatgagtacttgaaagtgaaatggggcaattaaacaagataaaaaagactaaaacgtgagacatctcacaacgggtcacgggcaagaagtacacgagaaatcgaacgtgagtggggaggcagagacactcagctgactgcctggcctgacgctcgctcacaaaacgctctcactctcttcctcgctttgcccgctctccgccccgggtcgtcagttcggtcgatccatgtttgttcatttttataggtgaaaatttatgtaagggaacggaaaatgtaaagtgatcgtgggaaaatagaaaaacaattacattgtaacttttctggaccaagttgtacccagatgcaatatgtatatttttctcagaaaatactgtgttgggtttcgacaggatcgatttatcaaaagcaaacgagtgtgcgtctcaacgagcactaaagttcccaactagagcatccttgttgtggtagaactacatagaaatttttaattttgatttcaatagcttttctcttgttttctcaaaatttattgaaaaacttatttactataaaacgaccaacgacggatctggaaactacagtactccttaatgcaaaaggcaacgaaaaatcagccagtgacttattttttgttctggataaaaatcgggaatatttgcattttgaattcgcactgtatcgataaacaaaacaccgaagatcacgccaaaatgactattgtaactaacaggtacgagaaagggacgcttgttctacaaaaataattcaacaaattttccccaaaaaaatgtgaagtccgcaattctcgtagttttacgtaaatcaaaccgagcatgacactctgacaccacgtgcgcctgaagatgtgcctgcctaccatggatgctttacatttgctagttccatgacaccccatcctttcagcttccaagatgaaggagttcggagaaaattcgaaaaaatattgagaaaaataacccaaaacattctgaaacattgcggaaaaaagttagaaattatgtcgaatatatctgaaccaatcaacaatttcaaataaaatacaaaaaaaaattggaagaccttaaatagtctccgcccatattttggcttcaaatgaccgtacttcggaatatggccgatggccgtggcaagacctccaatcgtagttttgagcggtcagtaagtgaagattaaaataggaacagtaccgtaagatcagcccaggtgcggatgtgggatagaggaactgaaaataatcgaagaagcatgataactaagccacgtggccacgttcgtttttgcgatgttaatagatcgccacttcgtccattgtcgttttgtttgtactaagtctccttagcaattctctcgaaggcgggccattgctattagtaaaataagctaccaattttacctttcaatacattcattcactgatggttttcctatcaggtgatcatttttttgttcttctcaattacactatctaaaaatgatgaagtttttgcttcgcggctatttggttgaagtgatgatatatccattgattgtcgtctccacttgtgctctttttacgtcttacaacttctttttaagtgttttgcgtattcactgtttcatttattttttgcagaaaatgagcctgttcagcaaatttttcggaggcatgatgcaagaagctccgattactccacaagaatctattcaaaaacttcgggaaacagaagatattcttgagaagaaacaagaattcttggagaaaaaaattgacgacgtaagttggaagatcagttttggtcgaattaatcacattaaaaagtgctgaaatcgaaatttttaaactctcgagtctcaagtgactgtgacgtaattaaaacattgctcagcatttacattgtttactgacgtcttttcgaagtttagtcgagcaaatccaaaaaagagcaataaaaatttctgctacgatacgtttgggaaattggaatcatagttttttaaactccatttttcaaaaaatacattattagaaaatcagtaagtttcggaaattatttgagaaacgtttcaggaaagcaaaatgccgtgaagtatggaacaaaaaacaagcggatggctctccagtgtttgagtaggaagaaagctttcgagaagcagttgatccatattgacggagttttggctactctcgaacatcaggttggtatataaaaatattagagaaataaattgaataacacggtttttcttccagagagaaaccctcgaaaatgcttcaacgaatgctgaagttctcacggttatgaaacttgctagcgatgcgttgaaagcggttcataataacatggatagcgaccaagttcgtgatatgatggataacatagatgaacaacgagaagtggcgaaggaaatcgcggatgctatttcaaaccctggctttaacaacgcaattgacgaggccgatttgctgcgcgagttggtggatcttgaacaggttcgtctataccaccaacatcgtgtaattattagaaaatataccaggaagcacttgacaaagatttgcttgatgcgagagctcccccagtcacgcttccggatactcccaatattgcacttccagcctccagaccgagagctaaagaagctgacaaggatctagaagacctcgaaagttgggcaaactaacttctctaagtcactttcatatttaattttcggctatttttgtttcatttgcatccccttcatcaatcctaccattctccggagattctcctaaatcaactttctaattacgacaaattcaaatagttgaatgatttctgtttagccatttcattcgaaacaaatttccccaaggctacgatcaacactcatcaaaattgtaacatattatcgagctttttggaaatttgtcattttatacatcttggtccctttctccaaaatcttccaagcatgcattaaagttccaacttttattaaaaattcattctggcaaacatgttatttgtaccggttgaaaacgaaaaccaagcgagaaatagttacatctcagatctccctaacgatggctcaacccctttgacgctcatttactaatgtttatacttttgctcatttactaatgaatggctcatttactaacttgctgagattttttaatttactactgctaattgtaagatatatatcatttatcatttactatatataaagcgcttattccgtttgtccatagtttgtagtctatgtagtctttgtagtctgtgacgttttggcttctggaaggatagtgagttgggcttagtgtagggatatagggggtactgtagtggtacaatagtggtacggtaggagtactgtatgattacggtagtttcagaaaaattagttttcagctccagaagtcgggggccgcgccggaggtgcggtccacggctggttttacataaggtagttccaaaaaatgtcctacttccaattactcataactcagttagcgcgctatagctatagcgtttgagtttaaaaaaattgtggccaactgaaatgctgtttgtcagagatgcgagctctaaaagatgatcgaaatattctatttctgcggatctagaatatttcgatcatcttttggagctgacatctccgcaatcgctaaagataactaaaaggtaccaattaacaaaatgtgttttacaatattgccaacaacattttaggtttctttcgctgattgtttccttttggttttggtgatggtcccggagtggtttttttcgctggttctactattttttggatcggcaggctctgaacaattggttgtacaatcttcttcaacttcatcaaattatccagagttatgttgtcgcttctgctgtccaacatattcatgcatttgacggaactcttcaactttctgcattgtcactggattcttctttttcgattttattttatgaaaactttactatcataaacaatagtatttatcatgttacaaatcagtttggaatgatctccttcattcaaaattcttaatgatcagtcgattcactcttagagccacgaaaaatgtgggacaattgtttgagaagtgaaaaatagttattaatgttgcaattagttgtacatataagtaatacatgaaaatacatcttaaaaatacagttactactaggtattattgcttaaaattgtgttccaatctgccagtactatgagcgtaattcgttgatccaatcttcgaatagccgtgagcacaggcttcgccggcactgcacacaaacttcacgattgcacgatttgcagaggtagaggacgaacgactttcctgtaattggcgaaatattgttttaagataaagttagtaggaacgatcgtactgtttttagaacgagactgtctagctggtggccgcatcgagcattgatggcatccaagaccttgaacttcttcgctgaatgatatacgatgcttgaatatggatccactgaaaattgaggttatagtagattattgggagctattatgatttcacccatgaagaactgcgtcagtaactcgtttcagattctcgctatccttttcaccgctttttcgttgtaattctatgagaaaacggtagaatttggtgacatttgtcgagttaaacaattccacgaggcagacaaacatctgaaatttgcgttttttccacaaatgcataaactttcaataaaacaaaccgcttctagggcaacatcagctaaactgtgatcatgctcgtattcggcgtttagcgagaagcataaatggtagaataaatgaaagatatcggtaggttcgcgggaatccggattgtagtctttgagataatcaacgcaattttgtttcagattcgtcatcaggtatttgtcgcatagcctgagaactgtgcacacgttttgttctgaaaataaatttggcattcattgaaactacatcgatcatgaactaccatcaataacatccggatataaaccaagagaattgggagaaatgacagtgatcaacttgagaatatcttccggtgactcatcaaggatattcagctgctttatggcgccttcaaggaaaaacttgttctccatcaagatgcggaaataatccgaatttcttgcaaagattgctggatccacaaagtacttttgattaccaacaataataggccagtttcgaagcttgcttgtcgactcaaaatcgacctgaaagaaaaatcgaaaaattccaatttaaaaaacgtttgtttacgtaatcggatccttctaggaaggtttcatgacttgttgtcggctgcattagaatgacgtttacggggaaatcattatttattccgaaacgtgcttgggcttgttctgtttgctgaaattttgaaaggttctccgaatattaagcgaaaaaaacttacattaataatataaggtctcatagcgccgagtagctaaacaattaatatttgattacaagtttggaaagatctttctgagctcgatcaggaagaaaaacttcttgaaactttagaagatgaaatgtgtgctaccgtataaactttaaaggtgcatgaataaatttctccttttggtcctgcgacgattaaactttttaatcaattctctgggctagtttttattcaataactagaaatgttgtttatttttgttccctacttaaatcatatgttattttctttttcctttgtgtcttacaggcttttttagctgaagaaatagcaattttccgataaaatttgttgctctatgttaaaggcgcatgcatttatttgagagacgggtctcgcaacgtgctcactcctcggcccgatttgttcttcgtttgcgcggttttcaggcctttaaaagatagttccgtcgtttttttctcaatttctgctgaaataaggtttaattaaatttattttcaaaatcttggtaaacatttaaactcatatattcagaattttcattcctctttcacccagaaaaccgaatttcaatattaagattaagaacacatctagaacatgcaaaaaacacaattgctatctctctactttcattttaaggctgattttttgaagaaaaatcatgaaatacgtccattattgttgtatcccttgtttgcatccaaagttgactcgattgatctcttaaatgtggtattccgttcgaaattcgattgatttttagaagttaacacattcggaatgatgataattcgtatcaaaccaaaattgtcttcttttcgccttttttgtgcagtgtcagcattaaacaaaacgagaatattgaaagttacgtggcgtttgcatctctcaccacgatgacatcacgaaatgcagacgacaaagaccggtgaaaaatagtgcgctgaatggtgaaaacttgcgaagataacgtgttacgggttgagagagaaaacattccgcgagacaatgcttttggtgagaggcgcagatggttcagagaacactagagaaaaccgcgcctctgtccgctcacagccagccccatcaagcctcttcgggcatcgacgcatagacacacatcattttgccccaatttcctttcattccgtcaagtatttcgcaactaatcgttattgctcattacaatacacattttacagaagttcctcttcttctacttggtccgaccgcatcagataactgggagatccagttgtgcatgttcttgtgcccacacaaactcgcgcccatttacaattttatgatcgacaaccctcaagaaggtaagcatttaaacgtgttggccgtgcgtctcaaaaaattgttaaaaaacctggcgacacgcgtttttccacaatttcattccctagggcattttgtatttgaagtaattctattacgcgtacgcaatcggacgaatcctgcaggtttgttggtagtcaattttatcaagtcgactgcctcttatgctttctgaaaaaagagaatgacagttttcgctaagtagtactaaagcgatcttttatctttggcaaaaccttgatataagcattatcacagcatatcatgcagattgatttagagttaagcatgaaatgtgcaaggctaaaataaattacaaaataagtccatagtccattttagtaacagtatacatcagctgatagaatcacatgcgtaatgacaggtctaaaacattatcaaacaaaagacattacaaaaacaagaaaaatacaatataatagaacgactatttgaaatgagcgtagttaaattcggaacttcaatagattatcatacgcgcttttaaaaaaatgtgtgttcccttttctccgcgtttgcccgctacaaaccggtgagtcggaaggcataatcgggttgaaaaaaaagtatcaaacactgatggtgtcttttttagggaggttgtccagaaagagaaagaaactgaagatttgcgaatcgatagcgtcgtcatctctcgacgccagtgaagtcaagatcggttacaatagtgtatgcgattcccaaaatccacatatcaaccggactcgtgatatttatcatttgtaagtactaacaagagatgtgaacgtatttacactcaacattagcaaattccagaagaagatctaaacaaaaactatcgaaatggctctcaacgtgaaccgcgctgtcgctgatccattctaccgctacaagatgcccaagctgtcagcaaaagtcgaaggcaaaggaaacggaatcaaaacggtcatttccaacatgtctgagatcgcgaaagctctcgagcgtccgccgatgtgtatgtttatcgccagttggctcgccattggacacaaaaataaccattgtttttcagaccccacgaagtactttggctgtgagctcggggctcaaacgaacttcgatgccaagaacgagcgttacattgtcaacggcgagcatgatgccaacaagctccaggatattttagatggtttcattaaaaagtttgtgctttgcaaatcatgtgaaaacccggaaactcagttggtacgagatcattgaattaataatctgtctaattttattatttcagtttgtccgtaaaaataacatcaagagcaagtgcaaggcatgtggatgttcgttcgacattgatctcaaacataagctgtctacattcatcatgaagaatcctccaaagattgatgtcgatttttgtaagtatcgtttactaacatttttcgattgaacttatgcaaaattctgccaaaaattctatttgcattttaaatcctttcaattcgattttccgtgtgcttccagtgcatacaaacatgctaatttttggtttccagccaaagccgaacaaaagaatggaaagaagacatcgggtgctgacgccgccgccgccgtggctgccgacataatccacaacagcgacaaaggcagttcgaatgatgacgacgacgacgattgggaacctgaaccagtcgagccgaatggcatgctgtcggcgggaatgggcaagctcgtgctggacaaggatcttgagaagagcgaagaacagcgtctcgacatgcttcacacattcttcttgaaagccaaggaagaaggtaagaattctgagcattgataaaaagtattctcgttatttcagatagaatttctgatgccaagggacaaactgctctacgtgacgaagctgagagacttgagctgaagcaaaaagcatctctccttctcgcgaacgtttttcttgatgagaaagtaatcactgacaaacaaatcagcaaacaccgcaatcttctgcttcgcttcacgttgaatgacaagaaagctcaaagatacctgttgggaggagttgagcaagtaattcacaaacatgaagcggaacttctgtctaaatcagctcacatcattaagtcattgtatgatgaagatgtctgcgaagaggattcgcttatttcatggggagagaaggttagtaccaaatggagctttgtttcgaattaaagtttatatttacagccgtcgagtaagtatgtctccaaatcttttgccaagaagattattgagaactctcaaccagtgctcaactggctgaaagaagcggaagaagaaaccgaagaagagtccgacgatgagattgcggtaagaaatatcagatttgttttttttttttcaatggttggttttcagttcggaggagacgtcaaggagagtgaattccttcgtcaacagaaggagaaggctgctagagaagctcagcaaaaatcagccaaggctacaaacggcaatgctgctgctgcatccggagcaaatgatgaagaggacttggatattgatgacatttaattgtacagatgcttttttaaaatttacctgggctacttatgttttttgtgtatttcttcccatattcgaaccaattcaactaatttcgaagaagcctcagtttttttttgctttctccccctttcaatagtaagcatcatttcatttctgtcttctgtcttttctgttcctacgctgttttcccttcaccaaatccaattcatttattcgtaaagtcattactatttgttgttaatcgtaaacatttgggaatattcttgttcaattcagtcttatattacaaaaacacaatgttcaaaaaaaaagaatcacttcagatgggaacccgtcgaattcggcggtccgatggagaatacacattgttttttcggaaagttagcccattttcaaatcatcacccagctgatttcatttgcgacgaagcgataaattgtaaagagccgaaaaccttttgctgctcggaacagtactatatgtacaataaggcttcactattgatggattcaaaactgatggcagcgattctagaagcaacttgtccgaaaacaatgaagacaatgtgttctaaatggtcgttgaaaggatggaaggattcggtgtaagttttaaatcagtttgataataaaatatgtttttcttttacagatgggatgagaacaaagaagaagtgatgagaataggatgcttggcaaaattccgtgcttctcgccatcttcgttatgctctttttctcacaactggtagcaaactagtcgaatgtagtccgttcgataaaatatggggaatcggttagtttccaacggatcgtcttattcttccatcgcccatcacaatgcaatcagaatcttcaaactggaaatgttttgaaatcattgaaatcatctttgagctgatatggtgacggaagaaaaggacgtctgaaaatggctgaattattataggaaaagatatgcaagccgcacaatgggctccattgagctctggcaagaatctgctgggaaagattttggatggaatccgagaggaattgtgggatgattcaaattacaagttagctctggaatcagaaaattattattatataaaattactatttcagagatgaacgagaagaagtggagaaacgaatggaaactgaaagagattatctattcactgctatagagcacatggacttgatgtacaaagaaagagcaacaaaaagagtattgtaagaatcagaaaatctgcgtaattgtcgacagaaataacgtattccagattgttcgaggaagaattgttaactgatgatagatcctacatcacaccagatattcagaggctccttcccgactgggcttggccgccgatcctcgtgaaaaacgagcctattcaaccatcgctgcctgtaataatcgatttccctaggtacttgccttgatctttaatttatcagaattaactttcaaattccagatcatctccacttcgagcagctgaaatatcacgtaggaagagcacatctcattcgacaagcttgagtaaaaggcggtacctcaggagcagatcgagaagtctgtccaaaagcccggctcgaagacgctccagacatctttcccgaagtggatcccgtacaccagctcaacggcattccagaagatccgaaagtacatctcgaagacgttccggacggcactctagaagtcgatctagaagcccaccacgaaaacgtccggtacgccgatcaagaagcagatccaggagcaggacaccaaaccgaaattggacaagagcacggagcagaacaagaagtcaggctaaaagtagcagcactttaacctggccactgagcccatcgagaagcagaagtaacagtaatgaaaggaatttgaaagagaagaaagaccggaaaaagaaaaaatctgagaagaaacggaagcatcattctaaatccagaaaacaccgttctaaaagatccgaatccagagaagaacgtcacagaagacggaaggagaagaaaagagagaaaaagaagaaacgacgtcggagaagttccactacttcagattaaactttatttttgaaaactagtcataactttaaaagtcataacttttttaaaagtcataacactggtttaatatcaaatgtcttttcaaatattctctatttatttattcttcgtaattaaactgagattaagtactgggtatatcattaataaaattacgatactttgccgaataaatcagttataattacaatctgtctgctggtgaaaattgtacatgctattttcttgttcctcattcttttttcattctctgtaaggttttgttcgttttttggaaaattctgagagtagccggaaaaaaaaaaaaaaaaaactaaatacctacagtaatgccagaggcatatgctcaataattatcaaaaattagttttccgcggcgagacccatccccacaaaagtatgactcccttgaaagtcgtaaatgacaatttcttgaaacaagaacatttgtatattaacgaaacacaaaattccgagaatgcgtattgagcagcatatttgccgagccaaatatctcgtagcgaaaactacattaattcttaaaaacactactgtagcgcttgtgtcgatttacgggctctttgaattatcattgatttatcgatagaatatttaaaaaataaattcatttcgaaattagagcccataaatcgacacaaacactacagtagccatttaaagaattactgtagttttcgctatgagatattttgcgcatcaaatatgttgcgcaatacgcattctcagaattgtgtcttccgtaataatagacagtggcttcgctaaaaactaagaacaaagtaaattaaagtttttttctgttcacttcaaattttacacgatcttgaagcaaagttcaaaagagcatgaatcaattggaaagtgttcaatgcaccctacagatatgatttcggggcagtgtaaactacagggcacagacataaaaatttaaattgttgaagactaaaatataaacatatgaattcaagggtcataataaatgtatttttttaaataatatttattaaatgtatgcatacaattaaatacaacataattatcaaatacaaatattataattgcaacctgtcggacaacaactttgctgaggtgtcgtgtgacagtcagaatccttgtcacaccagctgaccggctcagagacgatacatcggaagttgagatgagtgactggtggacattgccgacgcgttggagcacaacactcacgatatcgagtcatgtcgatgcagcgctgaaactcaggaaactatgtggaatttaggtggatcacccaaccagctgcccttcaccgcactgataatttggagtgcagtacatgtaatgggcagagcattgctgcatttgcatcacaatcaatgaatttgcaaagggcctggagattggcttggctgaaagagttgatattatttctattgatataataccctaaatttacgaaaattatgctaaattaggattttagttataatcctcgtcacatctgatctctgaaaacttaaaaatatcctttttggtagtgtggcaccaaattcgtgctgtaacagagaccaaaaacactactttttcgacatttcctctccttgcagcgaaaaataaaattttttgaaaatctgtgttttctcatacccggaaaaaaccaacaaaaacggccttgttccaaaggcggtgagtatttctattttatgaaagtggccgagatttctctttttctacgccaagtagttaattcttcgcggcaagacccatcaattttctaacctctaatctctttttcaacatgaatatccacgtcatcatagaatttgcactcgggcttatagatttggagcctttgaaagtatatgcaccagtctatatgggtgttgggaaacgaataggcagtagttttttggaccaattgtagaatagacagtagtaatagggaagaatataagaatttcataattcagatttcaataaaaaataaatttaattgagaaaaaaaacggttgatattcttttgtttaagcagacaagtatgcggaagtgaatcttgagcacctcgtaaatcacgggaggcgtacttgtacagaagagagataagggattaagaggcgcaagctttgccactttgaagttaaaaaataaagaaagagacatgcaaattggtggacaaatagcggaaggttagcgggaggtgggaggggggacaggtgcatgtaacacaatggattttacaataggaatattgaaaatacgcatatgggaaatcggaacagatatgaaggtgtcaatatttgaggtcaactgtctggtttttccccgatttttgaattttttgaaaaaaagtgcataattcacagattgaaattggaaattggtcgagaaaagaataaggagtgttatgaattgatggtggcaacaaaacacaaattctacatttgtaccaaaatgcccactaaaatgggcatattcgcacacattccacacaaattgcatacatattccacaatggggaatattttgaatatttagattaataaagatgaaataattgagttttatttgtaattaaaatatttttctgtttatcattaattgaaaatgttgaattactttttaatagacgaatcatcaaagaacttgatccctgcattatcaggcaatcctacataacctttcaacgttgtcgttttaccaattgcaacatttctcgctactggaacacgcatactggaatacgatgacgattccaattggaagaatatattggtgcccggttggaagttaacaattgaattgttgttaagcgataaaggatacacattgataacatccaaaagttcagttatgtatatccatccgtataaatcttgcgatcttccattcaccaaaagctggtcgccatcttgtataggaatgaatggagttaaggatcccgtaacagtacgagttgtgagcgtagttccactgaaaattactaaatatttagttcaaaggttttctgttactactttttggttgcaacaactctgagaaattttagttttcaccaaaatttttcgattttgtacagaattgcacaatatattttggaatagcaagaaattgttcagtgaatgtcaaatctgacaaaaaaaaatttttttaaaaggtgcctatcaatttttaaaaatgttctaatattttgttggaaagtttcaataatttcactacatttactatttcttttttaggcctattttgggtattcaaaatattaaccacacgaccttcaatacaggaaaactgtcaaattttttttaaattatgaacaattaactcactttacattttgtcctccattccttgtagttaatataagacttcccaacgcttcttgagaactattcgaaataatataaatcttcgaatttcttcctactatatatcctagtgtgttgctcgttgcaacgtctagagtatccaatataaacccactagaagctgatataaagaaaaataatagaaatatatttttcattttttccaaatgactaaatgaccaacttcaagacattttatatgcttaaaatcacgtcacagaactataatcatgttgatttttgatagaaaatgataagaaatgcgaccaaaatgtgtattttctccgtttgtcctctgaatgagtcaaattcacgtaaaacttggcatttgtcacagtgtgtcagacacaaggcacatatgtatttaccggacttttcaagactttattattattgagatcaaaccagattacagaagacgggagaaaggtaccaacaaatatcagaatattgcaaaaaaaaattaaaaatttcaaaacgcaaacttcaaactaggagagctaattcaaactttgaaatcatgttccataaccggtagcatttgttcggtgacttgtttgacagcccattgaaggaagagaagtactcccgacaggctgaaacatatgaaatagtccaggccttccattagagaatgtgatgtttgaaggaagaacaatgggacgtagagtactccgaatagagcagtaagtccattgatgagctgaaacagtaaataatcgaaaagttagtaaatatgttcaaggaatggaagtaaaccggaattatccgagtatgggcgttttatagttttttctctttttttgacttcgtttttcatcctattaaaatatcatcggttttttcgagttccagaaaaaatatttaaaaaatcatccgaaatccgaacacaaaatccgaaggctactccaaggtaagttaaccctactcggcaaatctctcgtcctggagcgcggacggggcgcgactagatcacgggttcgcgctccagtcaccctttttttcgcgcttcttacgcgccacgtccgcgcttcaggaggagcgatttgcggagtaccttttatgcattcagactggtacttaaaaattaatcgatttttttaaaaagtgtcataaactttttctacgtctttttctgacacaatgttgaaccgtactagattgttgtaaacacggtcttcaaatttgattttcgcgaaaaaatttgaataatttttttctaacttttttctttttaaaatcttaccacacttagcaaataaccatgaagcacaacttcataagtggatcctatttttcgtttgaagaggcaaaatactgaaaacaaaagagctgatatggagcaagacacgtggatccagaagagtatacgcacaatcacactatccccttcgattttgacgcggtacagaattctggaatttttttttgaactttaatggattgcgattcaaaagaaaacgtagcttaatctccagttaaagctgattttcattgcaaaatgtatttagaaaaaactcacgctaataaggcggagagtattgtctgtagaaccgccatgattactgtagatgcatagagtgagaatgagcacatataagcgctcggctgtttttgaacgacaatcgaattggccgccatcatctcattcttcgacctcccgttttatttctgaaaatatatgacactttttaaatgaattgacagaaatctgatgctaactacattttaacttgtaggagtggttcaaatgattcataaagggaatacaatttctgaatgatcaaagaagaaagaaaaaaaatattggtgaatgtataattttttaggggtaaagtaaataaataaacacaaggccgaagattagcaagagtttggggataacccccgtgaagaaaaatatgaaaaaaaatggtttgaaagaattaaaaaaatcctttcaaatttgagattcaaattttgttcatctgttctgttcgaacattgagcagaagaagcttttaccaataaatccaaaatttgttaagagaatatagtttaaggatatcacccagttcaaaatagtagttcaaaaactcgagtcttaattttttcagtattcgaatttttacagtacattgatcgtttcgttatttgatcgctttttgataaaacaaaaaatagataatgaagctgccaagtttaaaaaaatcggggctaaggctaatggagcatacacggtatatcactacctggatattagttttagacttcatcagatatttagtcagaaaagtacgtcaagaagtcggatacgaaatgtataaatttcttaaaacttaaaacttcgagatatccagactgtggctctcaagcttcagtgcttggagaaatagtttaatagtcagaatatgttttaaatttcttaatttttctgaagaagtcgtaaaagtataaatgttgctagatcaaacactctagaaaaccttcaccacttgagaatactccagtctcaaattttccctcgacgcggaagtgtagaagggcgcgagattcagaagtaggtgaaaattagacggaaaactctctcaaaattgaaatcaatgaataggacaactgagacaatgtgcaggtgtatgtgtatgcacatggcacccacgtacacgcatacatcttatgttagagaagtacgtgtgctccgctcatcatgtcttctccttctcctacatctacattttttgctccgtgagccacgccgggaaaaacgacgacgacgacggcgacgggggacgactactcgactctaattggccctaaacgcaagtaaatttttaggcaatgtatgtttgcgagagttgagagccccaccgccacgaggagaagtgggggaagattccgaagagattccccctcctccttctgatcacctcgtctttccttttttgttccatttccgtgaaaaagctgtggaagggaggagaagaacttaccggctaaatggaaaaaaaggaactctaacttattctgactctacggaaataggaagcctacttgtcaattagaccgccctcgcacagatttctttttttttgtagatacaaatataaaaactaactgcgtgtgatgcagcagatatcttgaattggaaagtgtcagtgctcagagggaatagccaatcattgacagaaatttgactacttcagaaggaatcaactagaacatttgacgcctgaaacctaacaagaaaaatctataatttggagatccctagattgatgccaactttattaaaaactaagtatacttatatatatacgatttttttaaaaataaacctgattgtctgaatttctacaagattgcgaccaaattttccgtatttccaaaatctaatattaggggtttctactaaaattcaacgagaactcttaacattatggttattttaacacatggttcaccgccggctcaaacttcattcttagtcctctgatttttggtaaatcgacgcctacgtctcaacaattagtttgtgcagaaaataagtaaaaagagttgtgctccatcttgcacacatacacatcgcctgtaatgaagaggttcggagtcagatgactaggcgtagaaatgtgcgaaattcacggataacagagatttttgatgtttcatcagacttacacgttttggaagtatgaattgggtctagacaacggagtggcagatgttcggaaaattttgcagaaaagagaacctaagagcgttgatggtttggtgactaacgaacttaaaagaaaattggtcattgaaaattttaaaattttaaattttgcttgcagttcatctttctctattaacaaaaattattttgtagcttttctcaatttcaggcaattaaaacatttcaatttattcttctattatggaagtttatctctaattgaaactctccaattttgatcaaagaacaaacgttctcgttgtttgaaaaaaaaaacagttcttttttgaaactcgcgcgcaaattattaaccaatcatcctcgtttgcgcgcaaaattgtagaaaaaatcatttaaatttatcaaaaatagtttaccattctgatgagtttttcatatacaaaaatgccctggcaattgttgttttctctgaaatagcacataataattgaactctacccacataaagttcgttctgaaaaacaccttacaattattgtgattgagagccaccccaagagggattagaaaaacggatgtaatctgtataccttcgagattcgtttatttccttgtataaccaatagcaggaaaattacagctttttctaagtaagcggtgaaactagagagattctatagaatatgggcgttaataattgtatgttaaagttttagaataacacaagtccagagtaagggcaagaaaagtaatgagcaacggaaaccagcatgcaagacacccgaattccggttctcttctgaaactaaaagttgcgtgtactaaaccttaaaccagcagctggctagtctcaagaaataatagaaaaaaggaaggaatgaagatatgggaataatacaaattgaaaatgttgtgtgagctccgaataattttcaatatcaaaaatttatgaattgtgtggacggctgtgtgtgcgtgtgcgtatgcgtcggcaagaaaaagaagcgaccgaataagaaaatggttgattcagtgaacaaaaaaagagagaaagatatccaaacaaaattattcaaaactattatcaatcggtaggtattgctctagagcacacctttctggacactcagcagacatgcgtagagagggattatgtggtacatatagtggatggaggaacagatatttataaatacttatggaaaagaggatgaagataggatgaggtagatgaattgagaagattttaaaatgataatggatattgaatttgaataaggagattctaaattatccgaagaacacaaactatatcaagactacaaaataatctagacgagtcccagttttgcaaggtaaggattaatcttaaaaggatcttttaaatatttatttcaatgctcctataaattttaaaaagtaggtgcattctaatatgtacagtgattaggagatatgtgacgttacgtgaggtctcgataaagtacggtattcgagctaaatttcaaacattgtcaaggtagattcggtacacagccaccataaatgttccactaaaaatgtgttgtccttctcctttggaacacaaatctagctgctgaactttttcacttcactacatgtcaatgggattgatatgcatctaggacatttttttggttatcaatagtccgcatagcttgcgtaaccaatacaaccgattgtccaaaaaaatttgaacactacaaaacgtatttattattcggatacccgttgcatttcaatacacaagttgatacttgctgcccctcggggctctcagacactcattgactgaaaacagacgattgctcgtcgtcgtagtctgaaggctcggagagctgaggaagatatgaggacataatgaattgatgtgtgagaatgagaaaatgaaaaaggaaaaatgagaaaaaaaagatgatgaagaatgtacaaatgaataatcaagtagcaatgacgagaaaagaaccaggtccttttggcaggcaattttcgaaattttcagatcaaatttgtcgccattgcttctggattaataatggatgacgctttgacaatggtgctcaatacaagtgcaaacagattggtttgggatggcgtatagaaatagagccggtgagacgatgtgatgaagttctgagagacgagatgtgatcgaggcgtttgtagtcgaggcaaaccgaggccgcatatggggttccgataggcaatcggagaccagtgtccatctgaaagagataaaagttattcgagttgtgaatgttgcaaggaaaattaaaggtacagtagagacaatcgagacttttttcgggaggacgccatctaaaaactgtggaagcacgtggctttggtagcttgatgtcacagaagttgattccataagaattacattagaaagcttgcgacgctaaatggataaatctggtaacggcttcctaatagcaagttaagttttttcacaataaatttttcagaattgaatagatgcattttataacttacacatcgagtgggcacgttggtggacaagacaagccccgat

SEQ ID NO: 24 - DNA, 4434 bp, Unknown
  Description of Unknown Organism: Unknown
gccgccctcgccaccgctcccggccgccgcgctccggtacacacaggatccctgctgggcaccaacagctccaccatggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcggacagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattgatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcacctgcagtggccagaattagggaatcagaatcgaaaccagtgtaaggcagtgctggctgccattgcctggtcacattgaaattggtggcttcattctagatgtagcttgtgcagatgtagcaggaaaataggaaaacctaccatctcagtgagcaccag

SEQ ID NO: 25 - DNA, 2837 bp, Unknown
  Description of Unknown Organism: Unknown
agagagccagtccgatgtctgcagcctccctggccaggcctctcctctcctgccgcagctagtccccctcaggacagacagagtactggcgtcggtcaccattcacttgcaaacacaccaggtcacgtgaagaaacttcctggtgacactcaggctgtagctgtgcactcttcaaccacgaggttggttttctcctaagtgtcacaggtggagacaagatgctctgggcactggccctgctggctctgggcatagggccaagagcttctgctggtgaccacgtcaaggacacttcatttgaccttttcagcatcagcaacattaaccggaagaccatcggtgccaagcagttccgagggcctgaccccggggtgcccgcctaccgttttgtacggtttgactacatccccccagtgaacacagatgatctcaacaggattgtcaagcttgcaaggagaaaggagggcttcttcctcacagcccaactgaagcaggaccgcaagtctcggggaacgctcctggtgttggaaggccccggcacctcccagaggcagtttgagattgtgtccaatggcccaggggacactttggacctcaactactgggtagaaggcaatcagcataccaacttcctggaggatgtgggcctggctgactcccagtggaagaatgtgactgtgcaggtggccagtgacacctatagcctgtatgtgggctgcgatcttatcgacagtgtcaccctggaagaaccattctatgagcagctagaagtagacaggagcaggatgtacgtggccaaaggtgcatctcgagagagtcacttcaggggcttgctgcagaatgtccatctcgtgtttgcagattctgtggaagatatcttaagcaagaaaagctgtcaacacagccagggagctgaagtcaacaccatcagtgaacatacagagactctccatctgagccctcacatcaccacagatctcgtggtccagggtgtggagaaggcacaggaggtgtgtacgcactcctgcgaggagttgagcaacatgatgaatgagctctctggactgcacgtcatggtgaaccagctgagcaagaacctggagagagtgtctagtgataaccagttccttttggagctcattgggggccctctgaagacaagaaacatgtcagcctgtgtgcaggagggccgaatctttgcagaaaatgaaacctgggttgtggatagttgtaccacatgcacctgcaagaaatttaaaacagtctgccatcagatcacctgctcacctgcaacttgtgccaacccatcttttgtggaaggcgagtgctgtccatcctgttcacactctgcagacagtgatgagggctggtctccgtgggcagagtggaccgagtgttctgtcacctgtggctctgggacccagcagagaggccggtcttgtgatgtcaccagcaacacctgcctgggcccctccattcagacaaggacatgcagcctgggcaaatgtgatacgagaatccgtcagaatggaggctggagtcactggtcaccctggtcttcatgctccgtgacttgtggagttggcaatgtcacccgcatacgtctctgcaactcaccagtgccccagatgggtggcaagaactgcaagggcagcggccgggaaaccaaaccctgtcagcgtgatccgtgcccaattgatggccgctggagcccctggtccccttggtcagcctgcacagttacctgtgctggagggatccgtgagcgctcacgtgtttgcaacagccctgagccccagtatggagggaaggactgtgtcggggatgtgacagaacaccaaatgtgcaacaagagaagctgccctattgatgggtgcttatccaacccgtgttttcctggagccaagtgcaacagcttccctgatgggtcctggtcctgtggctcctgcccagtgggctttctgggcaatggtacccactgtgaggacctggatgagtgtgctgtggtcacagatatttgcttctcaactaacaaagctccccgctgtgtcaacaccaacccgggcttccactgcctgccttgtccaccacgctacaaggggaaccaacccttcggtgttggcctggaggatgctaggacagaaaaacaagtgtgtgagccagagaatccatgtaaggacaagactcacagctgccacaagaatgcagagtgcatctacctgggccactttagtgaccccatgtacaagtgtgagtgccagattggctacgcaggtgatgggctcatctgcggggaggactcagacctggatggctggcccaacaacaacctggtgtgtgctactaatgccacctaccactgcatcaaggacaactgccccaaactgccaaattccgggcaggaggattttgataaggatggaatcggagatgcttgtgacgaggacgatgacaatgacggtgtgagcgatgagaaggacaattgccagcttctcttcaatccccgtcaattagactatgacaaggatgaggttggagaccgctgtgacaactgcccctatgtgcacaacccagcacagatcgacacagacaacaatggcgagggggatgcctgctctgtggacattgacggagacgatgttttcaatgagcgagacaattgtccatatgtctacaacactgaccagagagatacggatggtgatggcgtgggtgaccactgtgacaattgtcctctgatgcacaacccagatcagatcgatcaggacaatgatctcgttggagaccagtgtgacaacaatgaggacatagatgatgacggccaccagaacaaccaa

SEQ ID NO: 26 - DNA, 4108 bp, Unknown
  Description of Unknown Organism:Unknown
agagagccagtccgatgtctgcagcctccctggccaggcctctcctctcctgccgcagctagtccccctcaggacagacagagtactggcgtcggtcaccattcacttgcaaacacaccaggtcacgtgaagaaacttcctggtgacactcaggctgtagctgtgcactcttcaaccacgaggttggttttctcctaagtgtcacaggtggagacaagatgctctgggcactggccctgctggctctgggcatagggccaagagcttctgctggtgaccacgtcaaggacacttcatttgaccttttcagcatcagcaacattaaccggaagaccatcggtgccaagcagttccgagggcctgaccccggggtgcccgcctaccgttttgtacggtttgactacatccccccagtgaacacagatgatctcaacaggattgtcaagcttgcaaggagaaaggagggcttcttcctcacagcccaactgaagcaggaccgcaagtctcggggaacgctcctggtgttggaaggccccggcacctcccagaggcagtttgagattgtgtccaatggcccaggggacactttggacctcaactactgggtagaaggcaatcagcataccaacttcctggaggatgtgggcctggctgactcccagtggaagaatgtgactgtgcaggtggccagtgacacctatagcctgtatgtgggctgcgatcttatcgacagtgtcaccctggaagaaccattctatgagcagctagaagtagacaggagcaggatgtacgtggccaaaggtgcatctcgagagagtcacttcaggggcttgctgcagaatgtccatctcgtgtttgcagattctgtggaagatatcttaagcaagaaaagctgtcaacacagccagggagctgaagtcaacaccatcagtgaacatacagagactctccatctgagccctcacatcaccacagatctcgtggtccagggtgtggagaaggcacaggaggtgtgtacgcactcctgcgaggagttgagcaacatgatgaatgagctctctggactgcacgtcatggtgaaccagctgagcaagaacctggagagagtgtctagtgataaccagttccttttggagctcattgggggccctctgaagacaagaaacatgtcagcctgtgtgcaggagggccgaatctttgcagaaaatgaaacctgggttgtggatagttgtaccacatgcacctgcaagaaatttaaaacagtctgccatcagatcacctgctcacctgcaacttgtgccaacccatcttttgtggaaggcgagtgctgtccatcctgttcacactctgcagacagtgatgagggctggtctccgtgggcagagtggaccgagtgttctgtcacctgtggctctgggacccagcagagaggccggtcttgtgatgtcaccagcaacacctgcctgggcccctccattcagacaaggacatgcagcctgggcaaatgtgatacgagaatccgtcagaatggaggctggagtcactggtcaccctggtcttcatgctccgtgacttgtggagttggcaatgtcacccgcatacgtctctgcaactcaccagtgccccagatgggtggcaagaactgcaagggcagcggccgggaaaccaaaccctgtcagcgtgatccgtgcccaattgatggccgctggagcccctggtccccttggtcagcctgcacagttacctgtgctggagggatccgtgagcgctcacgtgtttgcaacagccctgagccccagtatggagggaaggactgtgtcggggatgtgacagaacaccaaatgtgcaacaagagaagctgccctattgatgggtgcttatccaacccgtgttttcctggagccaagtgcaacagcttccctgatgggtcctggtcctgtggctcctgcccagtgggctttctgggcaatggtacccactgtgaggacctggatgagtgtgctgtggtcacagatatttgcttctcaactaacaaagctccccgctgtgtcaacaccaacccgggcttccactgcctgccttgtccaccacgctacaaggggaaccaacccttcggtgttggcctggaggatgctaggacagaaaaacaagtgtgtgagccagagaatccatgtaaggacaagactcacagctgccacaagaatgcagagtgcatctacctgggccactttagtgaccccatgtacaagtgtgagtgccagattggctacgcaggtgatgggctcatctgcggggaggactcagacctggatggctggcccaacaacaacctggtgtgtgctactaatgccacctaccactgcatcaaggacaactgccccaaactgccaaattccgggcaggaggattttgataaggatggaatcggagatgcttgtgacgaggacgatgacaatgacggtgtgagcgatgagaaggacaattgccagcttctcttcaatccccgtcaattagactatgacaaggatgaggttggagaccgctgtgacaactgcccctatgtgcacaacccagcacagatcgacacagacaacaatggcgagggggatgcctgctctgtggacattgacggagacgatgttttcaatgagcgagacaattgtccatatgtctacaacactgaccagagagatacggatggtgatggcgtgggtgaccactgtgacaattgtcctctgatgcacaacccagatcagatcgatcaggacaatgatctcgttggagaccagtgtgacaacaatgaggacatagatgatgacggccaccagaacaaccaagacaactgcccatacatctccaactccaaccaggctgaccatgacaacgacggcaagggcgatgcctgcgactctgatgatgacaatgatggtgttccagatgacagggacaactgtcggcttgtgttcaacccagaccaggaagactcggacggtgacggccgaggtgacatttgtaaagatgactttgacaatgataatgtcccagatattgatgatgtgtgccctgagaacaatgccatcactgagacagacttcagaaacttccagatggtccctctggatcccaaggggaccacacaaattgatcccaactgggtaattcgtcaccaaggcaaagagctggtgcagacagcaaactcagaccctggcatcgctgtaggtttcgacgagtttgggtctgtggacttcagtggcactttctatgtcaacactgaccgggatgatgactacgctggctttgtctttggctatcagtcaagcagccgcttctatgtggtgatgtggaagcaggtgacccagacctactgggaagacaagcccagtcgggcttacggctactctggtgtgtcactcaaagtggtaaactccacgactggtactggcgagcacctgaggaatgccctgtggcacacgggaaacacagaaggccaggtccggactctatggcatgaccccaaaaacattggctggaaagactacactgcctacaggtggcacctgattcacaggcctaagacaggctacatgagagtcttagtgcatgaaggaaagcaagtcatggctgactcaggaccaatttatgaccaaacctacgctggtggacggctgggcctgtttgtcttctcccaagagatggtctatttctcggacctcaagtatgagtgcagagatgcctagagagcagggctccagctccagcaatgtgctgcaaacaccccttcttagacacatcagtccatcttggcacttgtggcttttctgtcatttggcatttcctgtttcttgaccttaactgagtggatctacacctccttcatcagcaccaagtccaagtgtcttcaaaggagaaacatcaattgcactccaagagcttccagcctgctgctggaaaacatttggatgagatatgaggctcaccgtggagcgaagaccgagcattccgctgtgttgccttttcttgtttgtttaaaaagaatgacgtttacatgtaaatgtaattacttgcagtatttatgtgtatatggagtcgaagggagctttagagcaca

SEQ ID NO: 27 - DNA, 820 bp, Unknown
  Description of Unknown Organism:Unknown
tcgaccagaggaggggaggccagttcctctcccaagggtgccacacacccctccctgttcatcaccagacaggcccttccttcttagccatatgctaaccttctcctccctgggaaatttcctctgcaggagccaaagcagatgggagctggagttgctggagctcctggtctgtatgcagagcaggcatccaggaaaggagaagagagtgtgacaatccagcacctcagaatggaggggcctcgtgttcagggcggaaagtacagacgcaggcttgctgagggcctctggacacaggctggaccagatgctgtggatgtcgacccctgcactgactattggataaagacttctttcaactaagagaagatgcaaatcagcacacttttttctttgttctgccagcttccaggcctaagactaggttttgctgtctacagccaactattctattagttacaaaactcaatcattttattcagcaactggatgttgactgttaactagaagctctgtcctacttacagcactttggatcatcaaaaaaataaagtaaaatagaaaactgagaaaactcaatccatgaccagggagaacttacaggatgttagagacaaaacaagcagacacctgaaacaatcaacgcccaataaaacaaagtaggatgaaaattctcttagttctttgataacaatttgttcactcatagaaacattattaattggtagggtaagcagacactctgaaacaatgagaaaaatactaaaaattgacttgagttatttcaaattgcctcattgacctgttatatcataactct

SEQ ID NO: 28 - DNA, 2397 bp, Unknown
  Description of Unknown Organism:Unknown
ttttttttttcatcctactttgttttattgggcgttgattgttacaggtcccagcctgtagacatcttttactccaatttcctgaatagatagctttattccttcaaggtaatatagtgcggtggcttctggctgagatgtttgctgttgttttcttcatcttgtctttgatgacttgtcagcctggggtaactgcacaggagaaggtgaaccagagagtaagacgggcagctacacccgcagcagttacctgccagctgagcaactggtcagagtggacagattgctttccgtgccaggacaaaaagtaccgacaccggagcctcttgcagccaaacaagtttgggggaaccatctgcagtggtgacatctgggatcaagccagctgctccagttctacaacttgtgtaaggcaagcacagtgtggacaggatttccagtgtaaggagacaggtcgctgcctgaaacgccaccttgtgtgtaatggagaccaggactgccttgatggctctgatgaggacgactgtgaagatgtcagggccattgacgaagactgcagccagtatgaaccaattccaggatcacagaaggcagccttggggtacaatatcctgacccaggaagatgctcagagtgtgtacgatgccagttattatgggggccagtgtgagacggtatacaatggggaatggagggagcttcgatatgactccacctgtgaacgtctctactatggagatgatgagaaatactttcggaaaccctacaactttctgaagtaccactttgaagccctggcagatactggaatctcctcagagttttatgataatgcaaatgaccttctttccaaagttaaaaaagacaagtctgactcatttggagtgaccatcggcataggcccagccggcagccctttattggtgggtgtaggtgtatcccactcacaagacacttcattcttgaacgaattaaacaagtataatgagaagaaattcattttcacaagaatcttcacaaaggtgcagactgcacattttaagatgaggaaggatgacattatgctggatgaaggaatgctgcagtcattaatggagcttccagatcagtacaattatggcatgtatgccaagttcatcaatgactatggcacccattacatcacatctggatccatgggtggcatttatgaatatatcctggtgattgacaaagcaaaaatggaatcccttggtattaccagcagagatatcacgacatgttttggaggctccttgggcattcaatatgaagacaaaataaatgttggtggaggtttatcaggagaccattgtaaaaaatttggaggtggcaaaactgaaagggccaggaaggccatggctgtggaagacattatttctcgggtgcgaggtggcagttctggctggagcggtggcttggcacagaacaggagcaccattacataccgttcctgggggaggtcattaaagtataatcctgttgttatcgattttgagatgcagcctatccacgaggtgctgcggcacacaagcctggggcctctggaggccaagcgccagaacctgcgccgcgccttggaccagtatctgatggaattcaatgcctgccgatgtgggccttgcttcaacaatggggtgcccatcctcgagggcaccagctgcaggtgccagtgccgcctgggtagcttgggtgctgcctgtgagcaaacacagacagaaggagccaaagcagatgggagctggagttgctggagctcctggtctgtatgcagagcaggcatccaggaaaggagaagagagtgtgacaatccagcacctcagaatggaggggcctcgtgtccagggcggaaagtacagacgcaggcttgctgagggcctctggacacaggctggaccagatgctgtggatgtcgacccctgcactgactattggataaagacttctttcaactaagagaagatgcaaatcagcacacttttttctttgttctgccagcttccaggcctaagactaggttttgctgtctacagccaactattctattagttacaaaactcaatcattttattcagcaactggatgttgactgttaactagaagctctgtcctacttacagcactttggatcatcaaaaaaataaagtaaaatagaaaactgagaaaactcaatccatgaccagggagaacttacaggatgttagagacaaaacaagcagacacctgaaacaatcaacgcccaataaaacaaagtaggatgaaaattctcttagttctttgataacaatttgttcactcatagaaacattattaattggtagggtaagcagacactctgaaacaatgagaaaaatactaaaaattgacttgagttatttc

SEQ ID NO: 29 - DNA, 4100 bp, Unknown
  Description of Unknown Organism:Unknown
ggatccccccgctccgctaccatcttcatcgacctcacccaggacgacgactgagctccctcttcctcgccgcggactggggcgaccctgttgctgctgcggccgccgccgctcctgcccccacttcggctcccgctcctgctcctgctcccggccccactcctgttcctgttcctgttcctgttcctgttcccggtcctgctccggctcccggccccgcacccacctccgctcctgctgcgggtctccaggcccagacaaaataaaaaaagatatattttttcagtccgtctctcccgcccggtgtcttctatggctgagggagtctggctctcggggctctcgggtcggctgggcggctcggctggttggctggctggcgagatggaccgctccggcgcgcagcgtccgcggctgctgtgatgggtgggcggagcgcggaccggggattatatacacgatgtgcatccataattgatgttgtttgagaaaaacaaagtcataaagtggcactcagacagcactttggcctggcgcccggccaccatctgagtgcccaaccgggcccggcggttacatcacccccacatggaccatcacggcccattagcaccaattggccagagtgtcgggagccaccgctaattgcagtaacgcgcggctgccagactgcaatttaccgcgcgatactgcagtttactgcagccgcggtaaactgcagtacgcggcggccgcaggaaatctactgtagtatttggcggcggcgcgcggtactgcaactgtagtaaactgtagctgcagtagagttactgcagcgccatcgggccggtgtggccgccagggtaactgcacccgcagtaaatttactgcagccggactttgtgcgctgtggagaccgcgccgaactgggacccccccgactcccccccgactcccccccgactcccccccgactcccccccgactcccccccgactcccccccgactcccccccgactcccccgggacgcgtccgcgcctcgatgcgccccatcgcgccccgttccgcttcgccacgctccagttgccccgcccccggcacgtggcacgtatttcccccccgtaaatcaagagggattatgcggatgtctagtttatgtctcaatttcctctttccggagataaaagccgggacccccgcgccgaaaaaggatacaccagccgcgatgtcgccgctcgtggcggtgctggtgtttttttcggcggcgctgggggttcctggccccggcgtcgcgggaaacccccgtgggctcgatgccatcttcgaggccccggtcacgcccgcgccccccactcgccatcctcggcgcgaggagctggagtgggacgatgaggatcacccgctgctggacctcgagccgcccgtgggatcacgctgccatccctacatcgcgtactcgctgccgccggacatgaacgccgtcacgagcgtggtcgtgaagccctactgctcgccgccggaggtcatcctgtgggcgtctggcaccgcctacctggtcaacccctttgtcgccatccaggccctggccgtcggagagcccttaaatgaggcggccctcaaggagctcggagaggtggccgtgcacaaggactccctgccgccgctgcgctataatggagggccccccgccgagtaagagaccctgcggcctgccgcccggggtgcgcctcgtcgtgcctgccgccgccgccgcttctgcctctaacgccgccaccgccgctgcagcagcagcccccgccggggccggggccggggcctcgaagccggcccgaccccccgccgccgcccggcccgcgaagggcacgcccgcggcgtcggcggcaacaacagccacgggggccgacgcctccgccccggcccccgaccccggggcgcccacgtgggacgccttcgccgccgagttcgacgtggccccctcgtggcgcgcgctgctggagcccgagatcgccaagccgtacgcgcgcctgctgctggccgagtaccgcggccgctgcctgaccgaggaggtgctgcccgcgcgcgaggacgtgttcgcctggacgcgcctcacggcgcccgaggacgtcaaggtggtcatcatcggccaggacccgtaccacgggccgggccaggcccacgggctggccttcagcgtccggcgcggggtgccgatccccccgagcctggccaacatcttcgcggcggtccgggcgacgtacccgacgctgcccgcgcccgcccacggctgcctggaggcctgggcgcgccgcggggtgctgctgctgaacacgacgctgaccgtgcggcgcggggtccccggctcccacgccccgctcggctgggcgcggctcgtgcgcgccgtcgtccagcggctctgcgagacccgccccaagctggtgttcatgctctggggcgcccacgctcaaaaggcctgcgcgccggacccgcgccgccacaaggtgctcaccttcagccatccgtcgccgctggcccgcacgcccttcaggacctgcccgcactttggagaggcgaacgcgtacctcgtccagacgggccgggcccccgtcgactggagcgtggactgagtcgggcgtgcgcgcacaccgccggcggaggacgaggaggggggaggggggtgggatggacggaggagagcggatgatggagcccgcgctcgccggcgccccggccagcgcgctgccggtcctggcggtgctgcgcgagtggggatgggccgtggaggaggtcgagccctccgggccgtgcccggaggacgcggacgcgccccgggagagcgcaccccctccccgggagggggtgcgcgggagcgaagacggagaggggggcgtggaagacggcgaggaggggaaggcgacggagaaggaggagacggaagacgaggaagacgggggggacgaagggacgacgacggcggcggcgggcccgcgccgggcgcagcacgtggagtttgacacgctgtttatggtcgcgtccgtggacgagctcgggcgccggcggctgacggacacgatccgccgggacctggccgcggccctggccggcctccccgtcgcctgcaccaagacgtccgcgtttgcgcgcggcgcgcgcggcccgcgcggcgcccccgggcgcggccataaaagcctgcagatgtttatcctgtgccgcagagcccacgcggcgcgcgtacgcgatcagctccggtccgcggtgcgcgcccgacgcccacgcgagccccgcgcgcgcccgacgagcggacgggcgcggccggccgcgccggtgttcatccacgagttcatcacccccgagccggtgcggctgcaccgggacaacgtgtttgcggcgccatgagcaccttcggacgcgcgtccgtggccacggtcgatgactaccaccggttcctgcaggccaacgagacggccgcccggcgcctggccgcggcctcccgccgcgtctccaccggcgggggcgagacgcgggccccgcggtcctcgcgcggcccccacgacgatgaggcgcccctgcgcgccggcggcctgggcaccgcccgcgggcgctcgcgccagcgcggcgcgaccgagccggaccccgtctacgccaccgtcgtccagcctacccaccaccaccaccagcagcaccaccaccgctctcagcatccgcagcagcagcaacaacagcagcgggccccacgccgccgcggcagcgtgcacgcctcggcgacggccgcggacggacccgagtcgtgcgcggccgcacccccgcgccgccgcggcagcgtgcacgcctcggcgacggccgccccggcggtccagctgccccggccccggcaacggagcatcaacgcctcgacgacggccgccccgacgccccagctgccgagaccccgccagcgcagcgtcaacgcctcggcccgcgccgccgtcccctcgacggccaccctcccgcgcccccggaccccgtcccggggccggcgcgcgccccccgcctcatgctgttatcgcgatcaataaagggcgagcgcccacggaccagacaaaagacacaaccggttcggtctctctgtccgcgcacgcgcgg

SEQ ID NO: 30 - DNA, 38734 bp, Unknown
  Description of Unknown Organism:Unknown
gatcctcgtgaccgggtacaccgacgcctcctggacgccgctgttcgccatcgcgggcggggtcgtcaccgacatcgggtcgatgctctcgcacagttccatcgtggcccgcgagttccacgtcccgtcggtggtgaacaccaaggacgccacccagcgcatcaacaccggcgacctgatcgtggtggacggcgacgcgggcacggtcgaggtcgtcgagagcgcggacaccgacccgcagggcccggccggggccgccgggaccccggccggagccaccaccgactgaagccggccaccgccgcaacaccggaccacgaccgcccccgcgaggggcggaccacaccccagacgggagacgacccgatgatccccaaccagtggtatcccatcgtcgaggcgcaggaggtgggcaacgacaaaccgctcggtgtgcgccgcatgggccaggacctcgtgctctggcgcgacatcgacggcaacctcgtctgccagggcgcccgctgcccgcacaagggcgccaacctcggcgacggccgcatgaagggcaacaccatcgaatgcccgtaccacggcttccgctacggagccgacggtgcctgccgggtgatcccggcgatgggctccgaggcccgcatccccggctcgctgcgggtacccacctacccggtccgggagcagttcggcctggtgtggatgtggtggggcgacgagcgcccgacggccgacctgccgccggtggcggccccggccgaggtgacggacaaccggaagctgtacgccaccaagcgctggacccgcccggtgcactacacccgttacatcgagagcctgctcgagttctaccacgtgacctacgtgcaccgggaccactggttcaactacatcgactacctgctcctgtacggcaccccgagcaagttcggcctcgacggccgcgagcggtacctggccgccacccggatcaccaaccaccgggtggagacggaggcggaggggcagaccatccgctactccttcgaccactgccaggaggacgaccccaccaacaccacccactacgtcatcacgttcaccttcccgtgcatggtgcacgtgcagaccgagcagttcgagaccacctcctggctggtgcccatcgacgaccagaacaccgagcacatcctgcgctggtacgagtacgaacaggtcaagcccgtcctgaggttcgaaccgctgcgccgtctgctgccctgggcgtccctctacatggagaagtgggtgcaggacccccaggacgtccgcatcatggaacaccaggaacccaagatcagcgccggcggcgtgaacaagttcatccccgtcgacgagatgaacgccaagtacatctcgatgcgcgccaagctgatcgcggacgcctcggccgcgccctcgtcaccggcgcgggcggcggagcccgagccggaagcggcggggcggggcggatcagcggcccgtgccacgggcaacggcaggggagcggccggcggacgacgcggcaccaagcccaaggaggacgccgccgcgcgcccgtagacccgaagacgggggacggacaagagagagcgagagtgagagatgtacggcggatacgacgcgtcgaccggccccaaggccctggtgacggccttcaacaccgtcgccgtggccggcgccgtgtggttcctgttcggcggcgcggacaccgtggccgactggttcggcaccgacttcgacgaggcggtgaccctgcgccgggtcctgctggcgaccctgtcggtgctctacctgctgcgcttcatcgccacgaacttcgtgatgctccagcgcaagatggagtggtcggagtcggccaccatcgggatctgggtcctggtgatccacggcacgatggcgtacttcggcggcaccaacgacgccggcgtgagcgcgttcacctggctgggcgtcgtgctgtacctcctcgggtcctacctgaacacggggtcggagtaccagcgcaaactctggaagaagcgcccggagaacaagggcaagctctacaccgaaggcctgttcaagcactcgatgcacatcaactacttcggtgacgccgtgctcttctccgggttcgcgctggtcacgggcaccccgtgggccttcgccatccccctgatcatggtctgcatgttcgtcttcctgaacatccccatgctcgacaagtacctcgccgagcgatacggcgaggccttcgacgagtacgcgtcccggacggcgaagttcgtcccctacgtgtactgaccccgcccgtcacgcgcgtacggcggcctccccgggcgaggggggccgccgtaccgggtggcaaccacagatcccacagatccccacagatccccacagagcccctccacagaccccctccagagatccacagatcccctccacagatccgagacgaggcacgtatgaccggagacattcccttcggagaggccgaggcgtccctgaccgccgaggtgctgcgcgaggtcctggccggcggcgccgaggcgttcgcccggctgacctccgacgagggcgccgtcgacgacttcggcttcgacccggagctgaccgacgactacctgctccccgccctgcgcctgctgtacgagaagtacttccgggtcgacctggagggactggagaacgtgccggccgaggggggcgcactcctggtcgccaaccactccggcaccctgccgctcgacgccctgatgctccaggtggcgctgcacgaccatcacagcacgcaccgcaggctccggctgctcgccgccgaccttgccttcgacctccccgtcgtccgtgacctcgcccgcaaggccggccacgtacgcgcctgccccgagaacgcgctgcggttgctcggctccggcgaactggtcggcgtgatgccggagggctacaaggggctcggcaagcccttcgaggagcgctaccggctgcagcgcttcggccggggaggcttcgcggcggtggcactgcggtcgcggcgccccatggtgccgtgctcgatcgtcggcgccgaggagatctacccgatgatcggctcggcccccaccctggcccggatgctgaagctgccgtacttcccgatcaccccgaccttcccgctgctgggcgcgctgggcctgatcccgatgccgaccaagtggaccatccgcttcggtgccccgatccacacggacggcttccccgaggacgccgcggaggacccgctggtggtcgagaagctcgccggcgaggtgaaggacaccatccagcacacgctcaacgagatgctggagggccgcggctccccgttcgtctgagggccgcggctcccggttcgcccgagggcggcggctcccggttcgcccgaggaccgtccctctcgtccggggccccgcctcagccccccgccgacgatccccggcggcagatgctgcgaacgctggcgaaggccagaacggcgaggccgacgagcgtgacgccgccgccgaccagctccgcggacagatgcatgggatctccctcagggggacgacggacggtgatggtcatatagccatgcgaaccccgccgtccgcccgatccgcagccgcaccgccccgcgaattcacccgtagagcagaccggtgcggccgaggaggggtggcgattgggtggtcgcgcgttcgaacgcttacgatcctctgttgtgtccaaactgaccgacgtgcccaagcggatcctcatcgggcgcgcactgcgcagcgaccggctgggtgaaacgctcctgccgaagcgcatcgcgcttcccgtgttcgcgtccgacccgctgtcctccgtggcgtacgcgcccggcgaggtgctgctcgtcctgtccatcgcgggcgtgtcggcctaccacttcagcccgtggatcgcggtcgcggtcgtggtcctgatgttcaccgtggtcgcctcctaccggcagaacgtgcacgcctacccgagcggcggcggcgactacgaggtggccaccaccaacctcgggcccaaggccggtctgaccgtcgccagcgccctgctggtcgactacgtcctgaccgtcgcggtctccatctcctccggcatcgagaacctgggctccgcgatccccttcgtcgtcgagcacaaggtcctgtgcgcggtcgccgtgatcctgctgctcacgctgatgaacctgcgcggggtcagggagtcgggcaccctgttcgcgattccgacgtacgtcttcgtcgcgggcgtcttcatcatgatcgtgtggggggcgttccgcggactggtcctggacgacaccatgcgtgccccgaccgcggactacgagatcaagccggagcacggcggcctggccggcttcgcgctgatcttcctcctcctgcgcgccttctcctccggctgtgccgcgctcaccggtgtcgaggcgatctccaacggcgtcccggccttccgcaagcccaagtccaagaacgcggggaacaccctcgcgatgatgggtctgctggccgtcaccatgttctgcggcatcatcgcgctggccgccgcgaccgacgtgcggatgtcggagaacccggccaccgacctcttccacaacggcgtcgcggtcggcgcggactacgtccagcacccggtgatctcgcaggtcgccgaggcggtcttcggcgagggcagcttcctgttcatcgtgctggccgcagccaccgcgctggtcctcttcctcgccgccaacaccgcgtacaacggcttcccgctgctcggctcgatcctcgcccaggaccgctacctgccgcgccagctgcacacccgcggcgaccgcctggccttctccaacggcatcgtgctcctcgccggagccgccatgctcctggtcgtcgtctacggcgccgactcgacccggctgatccagctctacatcgtcggcgtcttcgtgtccttcacgctcagccagatcggcatggtccgccactggaaccgcaacctggccggcgagcgggaccagtccaagcgacgccacatgatgcgctcccgcgcgatcaacgccttcggcgccttcttcaccggcctcgtcctggtggtggtcctggcgaccaagttcacgcacggcgcctgggtcgcgctgctcggcatgtgcatcttcttcgcgaccatgacggcgatccgcaagcactacgaccgggtcgccgaggagatcgcggccccggaggaccccgaggaggcacagagcgacgacatggtgcgcccctcacgcgttcactcggtggtcctgatctccaagatccaccgccccacgctccgcgccctcgcctacgccaagctgatgcgctccgacagcctggaggcgctcagcgtcaacgtcgacccggccgagacgaaggcgctgcgcgaggagtgggagcgccgcggcatcgccgtaccgctgaaggtcctggactcgccgtaccgcgagatcacccggccggtcatcgagtacgtcaagagcctgcgcaaggagtccccgcgcgacgcggtctcggtgatcatccccgagtacgtggtcggccactggtacgagcacctgctgcacaaccagagcgccctgcgcctcaagggccggctgctgttcacgccgggcgtcatggtcacgtcggtcccgtaccagctggagtcctccgaggccgccaggcgccgggcgcgcaagcgccaggactggagcgcgccgggtgcggtgcggcgcggaccggcccaccaccaccaggaccgtgaccgtacgaaggactcctcctcgtccacgtagactggacggctgttgtccctgtcatccccccgttctctggagtcaccccgccatgcaggcagaaccgaagaagtcgcaggcggaacagcgagcggtcgcggagccggtctcggagccggtctcgctggtgggcgaggagtacgaggtcgaggtcggccccgtcgcccacggcggccactgcatcgcccgcacgtccgagggccaggtgctgttcgtccggcacacgctgcccggcgagcgggtcgtggcccgggtgacggagggcgaggagggtgcccgcttcctgcgggcggacgcggtcgagatcctggacccctccaaggaccgcatcgaagccccctgccccttcgccggccccggccgctgcggcggctgcgactggcagcacgccaagccgggcgcccagcgacgcctgaagggcgaggtggtcgccgagcagttgcagcgcctggcgggtctcaccccggaggaggccggctgggacggcacggtgatgccggccgagggcgacaagctgccggccggccaggtcccgtcgtggcgcacgcgcgtgcagttcgcggtggacgccgacggtcgcgccggtctgcgccgccaccgctcccacgagatcgagccgatcgaccactgcatgatcgcggcggagggcgtcagcgaactgggcatcgagcgccgtgactggcccggcatggcgacggtcgaggcgatcgcggcgacgggctcccaggaccgccaggtcatcctgaccccgcgccccggcgcccgcctccccatcgtcgaactggaccgcccggtctcggtcatgcgcgtcggggagaaggacggcggcgtccaccgcgtccacggccgccccttcgtccgcgagcgcgccgacgaccgcacctaccgcgtcggctccggcggcttctggcaggtccacccgaaggccgccgacaccctggtcaccgcggtcatgcagggcctgctgccccgcaagggcgacatggccctggacctctactgcggcgtcggcctcttcgccggcgccctggccgaccgcgtcggggaccagggagcggtcctcggcatcgagtccggcaagcgcgccgtcgaggacgcccgccacaacctcgccgccttcgaccgcgtccgcatcgagcagggcaaggtcgagtccgtcctgccccgcaccggcatcgacgaggtcgacctcatcgtcctcgacccgccccgcgccggcgccggccgcaagacggtccagcacctctcgaccctgggcgcccgcaggatcgcctacgtggcctgcgacccggccgcgctggcccgggacctggggtacttccaggacggggggtaccgggtgcggacgctgcgggtgttcgatctgttcccgatgactgcgcacgttgagtgcgtggcgattttggagcccgccgcaaaggggctctgacctgcatttttcttggctggatcaggagcggcctgttgcgctcgacctgttctccaaagcgcacgacgtagagcttgcggaccgctcgtgaaagccgcctgacctggcgttgcacgagcggtgccgcgatgtcggcgtggtcggcccttctcctggcgcgaagggaaaccgaaggtcttgacgctcgggtgacgctatttctgaagggtcgtcaccgactggggaggcagggccctgcctctcgcgcccgatgaagcaggttctctctgctccaggtaatcgtcgagggtgccctgacggatcaggtagacggtcagggaccgcagggtgcagggcgtcgacgcccaggtcgaggatcatcagggcgctgtcattggtgatggcgaaggcgccgatcacaccgtcgacggaacaggttgcgttcaggagtctgtgcgggcgccgcaccggcacggtctggaagctcggctccgaccgcagctcgccaccagtccgagaggagccgatactgtccggtgccgggtgacccttcgtgcaagcgttgctgcccccgctcggcagaccggggcagcaacgcttgcacgatcggccggtactcaacgggatcgtgtggagtttcggaccggaacggcttggcaggacgtgcccgagcggtacggctcctgggccacattgcacacccgcttccgtcgatgggtgaaaggcggcacctttcagtgaaagggggttccgcccccccccgggaccttgcgcccaccgtcgccgaccggctgatgaaccggctccgcgctcccgccaccaacctgacccgacgtgagaccgaagtcctctcaccggtcgccgacggactgtccgaccaggccatcggcgcacgcctccacttgaccgaaggcaccgtcgatatcacctggcctgcatctatgccaacctcggaaccgactcgcgcaccgccgctgtggccactgtcaccgccatcgacgacctcgggctcatccgccgctgaacagtatgtggtgggcggtgtttccgttctccacgacttcagcggcgtccggagttgtggtgctggctgggcttggtgcccgctcctctgaacccatgtgaacgcccacggccagttcgagccggacacgccccccggacctggcctccgccgcggccagaatgcccggcccctgcacctggtctgcctacctgtacaggcgagggcggtccctcggagccactgcctgtaactccgaggggccgcccttggccgactcggcggtcaccgcggacgcggtgcggtcaggaggcaccgtctgcgtcatcaggcgcggctcggccgagcgagttattccggcacccgtgggaccagaaatgtcagccctgcgtgaccgcttcgaagaccgtgacgcggttgtcgtcggagagcgagtgggtggggtcggcgtgcgcggcgcggaatgcctccgacgaggtgtaggcggtgaatgcggcgtcgtcctcgaagttgaggacggccaggtagccgtgtgcgcccttgcggggacgcagcagccgtgcgttgcgcagccccggcacgttggaaagggtggcgcgcatgctggcggtgcagttgttctcgaacgcgccctgggcgggcgcggcgacggtgaactcggtgacggcggtgctcatttctgtcttctctcggttgttggtgtgatgtcggtggctgtcccgcccggccggggcccgcacggcgatggcgatgatgtgcaccgcgtgtccgatcgagttccgttgcggggtgcggttacagggctggagttgggctcgggtccgcggtcggctgagggagcctgcctgtgcggcgggcccagatttcgaacgcgatagtcatgaacgggggtacggcggccagcagggcgaagatcgttgtccggcccagccgccacttcagccggatcgcgaccaggacggtcagggacacgtagacgatgaaggcggcgccgtggagggtgccgaagatccgtacgccgagttcggtggtttcggggatgtacttgaggtacatcccggccagcagacctgcccacgtgcacgcttcgatgatcgcgatccaggtgaacgcgcgcagcagacggctggtgccggtttcggcagcccgtgcggccggcgcgtcggcggaaggcggttcggaggtgggcgtggagggtgtctgcggggtgcctgggcgtgcgggccacagggcgcggttgccgagcaggcggacgatggcggggaccaggagcgggcggatgaggaaggtgtccagcaggatgccgcaggccatggcgaagccgaactggaacagttcgcggatcggctgggtcatcaggacggcgaaggtcgccgcgaggatgaggcccgcggaggagatgacgccgccggtgcgtgtcagtgcggcggtgatcgccttcgctgggggctgggtgcgcagttcctgcttgaaccggctcatgatgaagatgttgtagtcgacgccgagcgcgacgaggaagacgaagatgtacgcggtgacgcggttgccgatgccgtcgtcaccgaggacggtcacggtgaagaaggtggtggcgcccagggtggccaggaacgacaggagcagggtcgcgaccaggtagagcggggcaaggagcgagcggagcagcaggacgaggaccacggtgacgatggctaggaccagcagcacgatgagggtcgtgtcgcggtcgagggcggagcggatgtcggcgttctgcgcggtctcgccgccgatgagcaccgtggcgtcctggacgccggcggcctgggctgcggattgtgtggcctgcttgaggggaccgatcgcgtcgagtgccttggagctgtaggggtcgaggtcgaggatgacgtcgtagaagacggtcttgccgtccttgcccatgcgggggtctgcgacacggctgacgtgatcggcgtcggtgagcgcggtggcgatgtcggcgggtgcggggctggagcgcaggttgtcctgggaatggacgacgacggtactgggggcgatctcgccgggcccgaattcctcccgaatgaggtgctgtccgtgctccgactcggtggcggcgcggaagccgctgagggtgttgaagctctcctggtagccgagcagtcccgcgctcagtaccaccaggagtgcgatcacggccgaggccaccttgacgggggcccgtgcgaccagggcggcgatgcggtgccagatgcctgcgccgcgactgcgttcggcggccttgtccacgcccccgggccagaagacgctcctgcccagcaggaggaccagggcggggatgaaggtgaacgccaccagcgccatgacggccacgcccagagcgaggtacggtccgaagccgtgaagtgccggggagacggccacgagcagggcaaacatggcgagcacgatggtcgaggcgctggcgaggacggactcggcggtgcggcgcacggcggcctgcatcgcgcgggcgcggtctggctcgtcgagcagggtctcgcggtagcgggcggtgatgatcagcgcgtagtccgtgcccaccccgaacagcagcacggtcatgatcgaggcggtctgggagctgaccgtgatgactccggcgtccgcgagaatcgcgccgagagtctccgccacgcgcatagccacgcccacggcaagaagcggcacgagcgccatcaggggcgagcggtagatcgccagcaggatgatcaggacgagcacgacggtggccagcagcaggactttgtcaccgccgctgaagaccttcacggtgtcggtggcgatcccggcggggccggtcaccgcgacgtcggcgggcccggcccggtcggacgcgagggcacgcacctcgtcgaccgcattctggaaggactcgtccgaggggctgccctccatgggcacgatgaccagctgagcaccgcggtcctgcgagaccaactcggccgcagcgtcgggagcggtcaccgtggagaccacgctcacgacatggtcgggtcggctggttccggaaagggccgaggtgatggcggcgaccgattgcgtggcgctcttcgcggcgtcggtgcccttgccgcggaccacgatgatcgccggcgtcgcgtcctggcccggaagctgggcgcggacgagatcacgggccttcatggagtccgaggcggcgggcggcaggttggcggaggcgttgtcctcgacggattccagggccggggcgaccccggcgaggaggcccgcgatcaggacccagaaggccaccaccacggcggcgcgcttcttcgatcccaggagacatcgcagcagagcgggggagttcatcggttgcatcgggcagccttcggcaggaagtacggacagaacttagcgacagggtgtctctaagttgcgtcaagctaacacgccccctcggcctctcgggcgtgggggtaggttggcgggagacggcacagcgtccgaggtgaagcggagaaaatgcccaagattgaagccggcagcgtccgggagcaccgggcgcagcggctcgcgcagctgattgacgcggccgaggagctcctggaagagggcggtgccgaagccctcacagccggagcggttgccgcgcgagccgggatcgcccgcaacagcatctaccgctacttcaactccatcgacgacctgctcgaactcgtcgtcacccgcgaattccccgcctggatcgacgcagtggagcaggccatcgcggccgagaccacacccgccgcccaggctgccgcctacgtcagggccaacctcgaacaggcagctcgcggcacccacggctggcgggccgcgctcacgcgcgactcgctctccccgtcggcgcgggagcgggtgaggaatctgcacatctcgctacacgaggcgctcgcccgggtcgtgcgcgaactggggcagccacagcccgagctgaccgtggcggtggtccaagcagtcgtcgatgcgtgcatccgcagaatcgaccaaggcgacgatctgacaaccgtgtccgacttcgcggccggagcgacgcgtcgactgctcgcggatgacgacttgccacatcacccgtgacgcaccccgtccaggcggctcgcaggcccgtcgacagcgaagccccggcagaacgagccggatcttgagccgcaccggagcgtgacgcagaccgctggtggctcatgcctcgtctcatccgatcttgccaccgggcggccgaccggtcagtgcccgacgcccatcgattacgacgtccacgacccgaaccagcgcgttcagtgcgttgacgttcgtggtgcgctcattggtcacccggcctctgggggtcaccagcgcttttagggcacgagactcgacggtggcgcgtgataccaggcaggcatcatgaccttatggcgatgacactccggcttcccgacgacctggacacgaagcttacggagcgggctcgtggggagggttgcagcaagcaggaacttgccatcggggccattcgtgatgcccgggaccgggccgagctgaaggtcgatgacgttctggccggtctgatggacagcgatgcggagattctggactacctgaagtgagcggcgtgcgctacctccagatcgacgagatcctggccatcgtgcgcacggtcaacggtgccgagcacagcgtgcgtgacatgggcctccttgtgtcggcgatcgaacggccccggacgaacgtcttcggagccgagctgtatcccaccctgcacgagaagccgcggcactactgcactccgtcgcccgcaatcacgcgctgatcgacggcaacaagcgcaccgcctggttcgccatgcgcgtcttcctgcggttcaacggcgccagcgccagtaccgtcccgccccacgggcgccggcccgacggacccgaggcccgtcacgcgctgctcaccagcagccctctcctcagcagcgcactgggaccggcgctgctgatcgccctgtccgccctgggggttctcgccctggacacggcgttgtgggtctcggtggtcagtgaggtggcggcgccggcccggtggggcttcgtgggcgggctgcgtgtcggcgccgggcgtctgggagccctgatcgccggcgtactcaacgccgtgatcggtcttggcgtggtcgctgtcaaactcatcgccgggcactgagagggcctgtggtggtgttcgcggagcgcatacggtggcagaccggtcggaatcctcggcgccgcggccggagcggtcccggcaccccggcgaacagccgcacgtccccgtccggtcgggtcaggtccgagccgtcagatccaggtcagtcgccacaggcgcagaagcccggtgccgtccaccgcgtactggccgccgcccacgtcctccccggacaccacgaagtccttggcctgccacagcgggacgacgggcacgtcgcgggcgacgatccgctgaagggcttcgaggtcggcttcggcgtcgctccggtcggcgaagcgctgactgctcgtgatcagccggtcggcggccttgctgccgtaccccgtcgccatggtgccgtccgtgccgacgagaggaccgccgaaggtgtcgggatcggggtagtcggcgacccagccgacggcgtaggcgtcgagctctccctcggcccagctcttctggaattcgtcccattcatatcctctgagggtcaccttgaacagcccgtcggcctctagttgctttttcacctccgcagcctcctcgtgggctgatccgcgtcccgccgcgtaaccgtaggtgaaagacagcgggatttcctcaccggcctcgacgaggaggcggcgtgccttttcggcgtccttgtgagggtagttgtcgaagaaggaggtggtgtggcccgtgatgctcgtcgggatgagggagtagagcgggtccacggttccgtcgtagacgtcgtaggaaatccggtccctgtctatcagccaggccgccgcctgccgtgcgcgtctgtcgtgaaacggcttgccgcggcggttgttgaggtacaggtttcgagtctccgcgctctgcgcctccgtcacgcgaagccccggatcgctcgggttcagatcggcgagcatttcggggggaagctgtctgagggcgacatcgatgcggtgggatatccaggcccgggcgagtgagtcgggggtgtcgtagaagtggagttcgatcggccggccggtgttctcggcggcgcccttgtaccgagggttgggcgagagggagatcttctcgcccttcgcgtaggagacgacgccgtacggtccggtcccgtcgatccggccgtccgagcgcagggagtccgccgggtacgtggtcgagtcgacgatcgagcccgccccggtcgtcagcttgaaggggaacgtggcgtccggtgcggtcagtcggaaagtgacggtccggtcgcgggcgtccatcgactcgatggtgtccaggagggacgacggccccacgtcggaatctatcttcttgacccgttcgaacgagaaccggacgtccttggctgtcattctgcgtccgctggagaaggtgatgtcatcccgcagccggcatcgataggtgcgtaggccggaatcggtgaaggagcagctttcggctgcgtcgggaacgggctccgccactccgggctccagggtcagcagtgtctggaagacattgctgtacagagtggtcgagccggagtcgtagccgccggccgggtcgagtgacgtcggcggttccgtcgtcccgaccttgatggtggtgccctcctggtcgtccgtcgggtaaagcagcagaccggctgccagtgccgtggcgatcaccgtgggtgtgatgacggacgcacggatgtgcgcacgaataggtctcatgaggctcgtcctcgcaagatcgagacgaacaggaattttcgtacccctgggtggagagtgcgtcggccaagtatgcgcaggcgtcgcttccttcggagcccgacggcacttccggaacgaagtcttatgactgacacggtggaactgctatgccccgttcggcgagagggccgccaggggtcggcaccccctctcagcagccgttccgcctcgtctccggtggtcctgcggacccgcttgcgcgggtcccgcccacggtctcactcctcgatgccattccctgtgcaatgtcacctgtgccatgttccgtgttgcagggcgtggccatgccaagtcgggaggtcgttcgtcttccgtcaggtggcagtgcggtactccgtttcccacgtcctctcccccttcagtcggccgtgctccgcacggccggatccctcatgggaggcgctgtgagaaagtcactggtacggcgaggtctgggggcggcgctgccgctggccctgaccgtcgccatgagcgtgggcctgctgtcgcagccggccggcgcagccgggaacaccgggtccgtcgtgcacgtcgcggcggacgacccggagcacgcgggacccccgcccgtcgcgcagtcccccaccgccgagacggagcacgtcgcgcagggacgcacgagggcgtccgagcttccgcccgtggccgcgagtaaggacgcgctcaaggaggtgtacggcaagaccgcgaaggcgccggtccgtccctcgaagtcgacggacaaggcggtcgccggcaagaccggcaactcccgtgcgcgtgccgccgcgtgcaacgtctccgacttcaccagccggagcggcggcgcgctggtccagcagatcaaggcgtccacgaccgactgcgtcaacaccctgttcaacctgaccgggaacgacgcctactacgccttccgtgagtcgcagatgacctcggtcgcctacgccctgcgcgacggctcgacgtcctacccgggcaacgcctccaccggtatgccgcagctcgtgctctacctgcgcgccggctactacgtgcactactacaacgccggcacggtgggcacctacggcagcagcctgcagaccgcgatacgcgccgggatcgacgccttcttcgccagcccgcactcccgcgacgtcaacgacgccaacggcgagacgctcgccgaggccgtcacgctcatcgacagcgccgaggagaacgcccgctacatccacgtcgtcaagcgactgctggcggactacgactccacctggaactcgtcgtggtggatgctcaacgcggtcaacaacgtgtacacggtgaccttccgcggtcaccaggtgcccgcgttcgtgagtgccgtgcagtctgaccccggcctgatcgacgcgctctacaacttcgcgagcggccacctcgcgctgctgggaacggaccagtcctacctcacgtcgaacgcgggacgtgaactcggccggttcctgcagcattccgcactgcgctccaaggtcagccctctggccggcggcctgctcaactccagctccatcaagggccggacggccccgctgtgggtcggtgtcgccgagatgaccgactactacgacaaggccaactgctcctactacggcacctgcgacctccaggcacaactggcccgctccgtcctgacggtgacctacccatgcagctccagcatcaccatcaaggcgcagcagatgacctcgggcgagctgtcctccagctgcagcagcctgcgcaaccaggacgcctacttccacaacgtggtccgtgacaacggccccgtcgcgaacgacaacaacagcaccatcgaggtcgtggtcttcgactccagcaccgactaccagacctacgccggcgcgatgtacgggatcgacaccaacaacggcggcatgtacctggaggggaatccgtcggcggccggcaaccagccgcgcttcatcgcctacgaggccgagtggctgcgtccggacttccagatctggaacctcaaccacgagtacacccactacctcgacggccgcttcgacatgtacggcgacttcaacgccaacatcaccaccccgaccatctggtgggtcgaaggcttcgccgagtacgtctcctactcctaccgcggcgtcccctacaccgaggccacgaccgaggcggggcgtcgcacgtacgcgctgagcaccctgttcgacaccacgtacagccacgacaccacgcgcatctaccgctggggctacctcgccgtgcggtacatgctcgaaaaccaccgcgccgacatggacaccgtcctcagccactaccgcgcgggaaactggaacgccgcccgcagctacctgaccggcaccatcggcacccgctacgacaacgactggtacacctggctggcggcctgcgcggccggcaactgcggtggcgggggcaccaacccgcccgggaaccaggcgcccaccgccgcgttcaccaccgccgtccagggcctgaacgtcaccttcaccgaccagtccaccgacgccgacggcaccatcgcctcccgctcctggagcttcggcgacggcaccacctccacggccaccaaccccgtcaagacgtacgggtcggccgggtcctacacggtcaagctgaccgtcaccgacgacaagggagccaccgccaccgccacgaggacggtcaccgtcggcagcggcggaggcggcggcaccgaatgcaacgggaccgacacccgggaactgggccagaactgccaacgcggcaaccagtccgccaccaccggcaactacgcctacctgtacctctacgtcccggccggcaccacccagctgaagatcaccacctccggcgggacgggcgacgcggacctgtactacagcaccagcggctggcccggcaccacgagctacacgcagcgggccacgggagccggcaacaaccacaccctgaccatcaccaacccgccggccggcgccaactacatcagcctgcacgccgtcagcagcttcagcggcgtcaccgtgagttccgcctactgacccacggctccgcaccaaggcacgaccctcacgacggcccggggcggctctccccgccccgggcggcgtccggggcggcggcaggggggagacctccgtcgccccggaccgagaacacatcgcccgcccgcacacgggcatccctacctcccaggaggcagagcgtgaagtcattacccgcacgcaggcgacgccgcgccatgtggtccctcatcatgtccgtcggtctcacctgcgcactcgccacacccgccgtcggcagcggtgaccagggcacgtcacggctcagcgcctcgcaacaggccgcggccggccaactcgcagcggaccagcacatctccacccaggaggcacagcggcgcgtactgcggcaggagcggctcaccggcgtcgcaacagcgctgcgtgagcgcctgggttcccgcttcgcaggagcctggatcgaccagaagcacggcggcaggctgaccgtcgccgtcacccggtcgacggccacggccctcgtcgaggcccggtccgctcaggctcaggcacccgacacgaccaccgtcgtcgtcgaccgcagcctgcggcaactcgaccgcatgtccgcaggactggcccaccgtatcgccgcagcgaacaagggcgccgcccacggcctgcagtccgcggtggtggtgcaggacaacaaggttcgtctggacctgccacggggcaagaccctcacccccgcccagcacgcagtcgtggagtgggcgaagcggaccctcggcgatggcctcgaggtcagcacctacgcgcatgcctccgaacccttctactgcggcggccagtactcgtgcgaccccccgctgcgctcgggcctggccatctacggcacgaacgtccgctgctccagcgccttcatggcgtacagcggcagcagctactacatgatgaccgccggccactgtgcggaggacagctcgtactgggaggtccccacctacagctacggctaccagggggtcggtcacgtcgccgactacaccttcggctactacggcgactccgcgatcgtcagggtcgacgaccccggcttctggcagccgcgcggctgggtctacccctcgacccgcatcaccaactgggactacgactacgtcggccagtacgtgtgcaagcagggctccacgaccggctacacctgcgggcagatcaccgagaccaacgcaacggtgtcctacccaggccgcaccctgaccggcatgacctggtccaccgcatgcgacgctcccggtgacagcggcagcggcgtctacgacggctcaacggcccacggcatcctcagcggggggccgaacagcggatgcggcatgatccacgaaccgatcagccgagcactggcggaccgcggggtcacgctgctggccggctaagcagcccgggcggaccgtgagtacgccgccccggtcacatcacgaggacgtcgaccgccgcacgcgcggtcggcgtctttccccgtgctccgctccgtccgccacccagcggactgggggcgggggcgtggcacgtcgtgcacgccgcagcgcggtggaacccgtcggccgattagaccgtaccggggagcgcctttccggctccgttcgtgggacgggcgggtgcgtatgcgcgcgtcacccatttctggaagtgcggagcctgcgacagcagttgccagtgggcgcgtacggcatgatggtgcaccacctcgacggccgacgcctcgaccgaatcccgccgccagacgagcagatgccgctgccacagcggatcccccgcgaggggtttaaccagtactccgcccaccgggcgcatggtgggctggacggcggccacccccagacccttggcgatcatcgactgcagttggtcgagcatgtggaactcgtgggtgacggcgggcctgaatcccgcggccccacaagcgtcgtagaaggcgccgggccagcccaccccgtcgtccgcggagacgaaccacgcgtcctccgacaggtcggccaaggacacctccagccggtgcgccagtgggtgatcggcaggggtggccacgaacaccgggacggttctgatagctcggtggtccagcttcggagagtgtcgaagaggcagccctgggtagtcgcaacccagggcgacgtcgagctcgccggcctctaggagatcgatgagttctccggtcgcgtacacactgctgaccgagacggtcagatcggggcaggcttcacggaggacgtcgagcaaggtgggtaccaccggtgtgttgatggccccgaggcgaagccgacgtgtcgccccggacgagcggggaggccgcagccgtgcgagattgtcggagagcgccaggatctcccgggcccggccgacgacctgggcgccgtaggcggtgagctccacgcccgcgctgctgcgcaggaagaccccctcgccgagcagtccctcgatgcggcgcagttgggtactcatcgccggctgggtgtatccgagcgccgcagcagcccggccgacgccccccgcgtcggctatcgcacacagcacgcgcaagtggcgcagctcaagttccacgggggcacctcgctccgggcgaacagagttccattatgcgccaggaggaaggcggtggggaatccgggacggcctgacgccttcggtcgaccagtagcccgagggttatggatgagccggagcctctggtatggcctggccggttgttcccgggtgaccgccgtggaaatctcggacctgcgtgttggtccgcagaggcgactgcggaagcctgaagcgcaccgccatcgaggagcgacatcatgcctcacacctgcatcagcttcaccgtcgaagcgaccggggccgcggttcaccgcgcccgccaccgcgtctccaccgcgctgagctggtggggagggccggtcgaggaagagctccgcttcagcgcggaactcgtgacctccgagctcctcaccaacgggctgcggcacgcgggcgggcccatgaccgtcgagttgacgctggtgcacgacatggtcgtcgtcgcggtcctcgatgacagccgggagctgccgcggcctcggcagacggaggcggacgacgagtgcgggcggggactcgccctgatcgaggacctcagtctgatacggggagtcgagaccacttcccgcgggaagcgctgctgggcggttctgccgctgcggacgccacaggagcgggctatcgagtcggctccggctgaggaggcggaccacggcttcgaggcagaccgggaacgctggtcactggctccccaaggaagcggactactggcgagtctgtttccggcgatgtgagttcgtcctcctcgggcggcccagtagccgacccagggcaggcgggcgtgcctgagggcgtgatgacgctcgtctgacgctctggccgctttcaagctgcacagcgagccgagaaacagcctttgacctggccttttctgcggctgcctcaggccgacatctttccgatgacgcaccacgtggagtacgtggcgattctggagcctgctggcaaggggttctgacctgcgcttttgttctcctgcggcgggcgcggcaagctcgtgcggggcagttgggtttcccgaaggccggtgctcgtgtgtccggccggcgggtgggctgccttcgtttcagtgggtgcgagagggcactcggacgcctgagccgagatgcggttcgttcggcaccatggggtccgcaggatgacccggtcagcgaccgctggcacctgtggaagaacctttgcgacaaggccctggccgaggttcgctcccacagcgcctgctggaccacagcgaacacaccccgcccggtcggcgtccatgagcagaccacccgcgaacgttggcatcagctccacgacctcctcggcaagggtgtcggcttgctcgaatgcgcccgccgcctgaacctgtccctcaacaccgtcaagcgctacccgcgcacccgcgatcctgaagccctgcgccccgtgaagcagctgtttcgcgaggtccaggagcagggctgcaccggcagcttcaccctgctctaccgcagcacccagggccgggcagaaggcgaccggcccgtcggagggtcgcggcttgacgctcaccgtatccatcactggaacggcgacgtctgatcccgtctgcccggggcttgggtcccggctgcggcccgtaggcccggctcaccccagcacccatcactgttcgagagtgattacctctccgccggacacatggaaatctgcatcggctggagtagacattgggcagcagtgtggttatgtttctcctgtaacccagaaggaccgcagggcccggcagagacgaactgccgggcagcagtacccgcagttgcaggacggtgcggtggtggagtgtcgaagccaggatggtgcaggacggcgacgggactgacgaccggaccgggcggcccgcagtggtcaggggccgccaccgcagtgcagtacccagcagcgaagtcagtgagcggtacctcggtgaaggcgtcggctgcggacgcgcgcgccgggaggttcggcagtggtggttccaagccagagcagacgcaggacgggcaacggggccgactgtcggacagtggcgctgtcacaggtcactgagaggttcgtgtcaccagcagtagagcagtaccagaggaaagaacggaggaaccaagcgccatcaggatcgcccgggcgcagttttgggcccgggtaccgcaggacatcgatagtgaggtggtctccggtcaagaaaccgcgatccccgcgcccccggcagcaggcaggtcgggtccgcggacacagaaggccggtgcagtatcagggccggcagatggtgtaggagttccttcggggccctggtgccgcatggcaccagggcccctccatgcgttccgcagagaggtgcagatgacagcagacgattcgtacggccgtctcgacgacgacgattaccccgcctacaccatggggcgggcggccgagatgctcggtacgacccccgctttcctgcgggccgtcggagaagcccggctgatcacgccgctccgctcggagggcggccaccgccgctactcccgctaccagttgcgcatcgcggcccgcgcccgcgaactcgtcgaccagggcactcccgtcgaggcggcctgccgcatcgtcatcctggaagaccagctccaagaagcgcggcgtatcaacgaggaactgcagaggcgcccggccggcctggtggacaaggccgagggctgaggccgcatctgccggccggtcctgtgagggctcgcctgccaagacgggaagcccttgccgcaacgagaagaggcaactgtccgcaccgatgtgctgggcccggtcctggctaggactcccgtcttcttgccggagcgatgcggctgtggacgcggaaccggacggcagtgtcgtcgggcgcggagcgcggggcgcacgtcgatggcgacaggaccggcgaaggtgtattcgtgttcggcggtgtgacggcgcacctggccggcgagggcggcggcgcaggtgtcacagggacatcggttccgacttccaccacccgtccgggttccaccagcgtgtcatccacctgatccaggctgccgcggtaggtgctcgacgtcgggggtgtacgggggcagttgtaccgttccgccgcgaggagtgacccgattgaccaccggcctgtggcgctcaggaacgggctggactgtcgcagtccgggccaactcaagcccgaccatgaggccgaccacggcgccgcgcgaccccgaccacagctacacgcgtggcatgaccaaggcggcacatgcttcgaacgagccatctcatgtgtgccggtatgaacgtgatcgacgtccccggcactctggtgcggacgcaagccgtctggggcgccacccacgactggctcgccgccccgcccccgcggcgccaccgtccgtcccgccctgcgtcgtcgcccgtgtggcgtcatgacggcgacagacttcctcgcgtatgggccgaccatacggccaacgccagaggtaaagcgctgtccatggtgagttccctgaacagaagggctggcgggacctcctttccaagaccgtgctgcaggagtccgtcagagcgcaggtaatcccgtgctgtccgcgacccagggctgtcctccgtctggccgagggtcctcgtcttctgggcgacatccctttagcgtgggcggtagccgccgaagggaggcgccatgtcggacgaattgacgggcccgttgggaacggcaatgcgggaggtcacgtttccggaccggtctcgcgggatcatcttggtgcgggctggaacaccgcaggccgaggccgaggcaatggccgcccgtatgtgggccgagatgccggaaggctgacgtgcccgaacgcagacaacccgtaccgtcctcacacgcattcccctgagccgtcggccatggaacggaaccagccgtacgaaccccggaggcgccgttgcggtctctgcggcgaggccggggccacgcagggcgaagaggccgcgccgcgttctgccgcctggcgcggctgccggctgttcacgagaacaccgagggaggagtcgcccgcctcttgcccggcgcgttgccgggtggagagcaggtggtgaaggactggctcgctgaaggcggccgaggcgacctcgtcggccggcctgaacggctttcactgtcccagcggcggcaggccgccgacacaggcatgctttgccatctccctcgctgtctactgaccccagcagcaggatccagtacggcgtcgcggcgctgccgcctcactcgcgcatcgatcggggaatgcggcatgtggtgagggcccggccggcgtgccggtcgggccctcacactgttttggtgtcggcgcgtttgtcgtgtcggtcagacggacaggtggggggcgccgagcatggcggaagcccgctgcaacggactgtcgctgcgcgcgggggcggcctcgggaagggtgcggcaggtgaagcccagctgggccatggcccttaggatttcgccggtgctgaagtcgcggcggtcctggcgggtgatgacctggccgacctgcttggcggggtagtggcgtcgtccgatgatcacggactcgccggtgaccggttcgggtttgacgcccttcatcgattccagcacgccgctcttggtcaggtcgaacgggaagcgggcaatgacacagcgcatgatgcctcacaggcaggagagttacggggccggccgccgtctggcggttcagcgggagagagcgaggacgcccagggcgctgccgtgttcgtcgaccacgggcaccagcccgagccgtccgaagggcaccgcgtcctcggcttcctccctcgtggccgacggtgagacgaagggctcgctgtcgtcggtgatgtcaccgaggcggagccggtcggtgtatcgggagctgtcccggacggcggtgagccgggcctgggtgaccaggccgacgcaccgggcatcctcgtcgcagacgaccagatgctcggcacgggcggcggccatcacggacagcgccacctcgacggtcatgtcgtaccagacctgtggcccggcggcgtccatgacgtcggccaccgtgccgcgcaatgggagagcgcctacggagcgatcctgcaactgtcctggcgtcaaggggtgcctcctgcgcagacgggcggggttcctgatcaggacggtcctaggcggccgcgccagccgtggacttgagtgcgggggtacgccgcgtcgccgaggcggggcggcgtcggccgcgtgaggtggcgccgcgcttcttggggcgttcagtcgccggggcggtgatgacgaccgggatgccggtcggggcctgggctccggtgatccggctgagggcctcgtcgcccgggctgacctgggtggtctgcggccggatcccggcttccgacatgagacggaccatgccgcggcgctggttcggggtgacgagcgtgacgacgctgccggactcgccggcgcgggccgtgcggccgccccggtggaggtagtccttgtggtcggtcggcgggtcgacgttgacgacgaggtcgaggttgtcgacgtggattccgcgtgccgcgacgttggtcgccaccagcacggtgacgtgcccggtcttgaactgcgccagagtgcgggtgcgctgcggctgggacttgccgccgtgcagggcggcggcccgtaccccgctgttgagcaggtcccgggtcagtctgtcgacggcgtgcttggtgtcgaggaacatgatcacgcggccgtcgcgtgcggcgatctcggtggtggccgcgtgcttgtcggcgccgtggacatggagtacgtggtgctccatcgtggtgacggcgccggccgaggggtcgacggagtgcacgacggggtcgctgaggtagcggcgtacgagcaggtcgacgttgcggtcgagggtggcggagaacagcatgcgctggccttcgggacgcacctggtcgagcagtgcggtgacctgcggcatgaagcccatatcggccatctggtcggcctcgtcgaggacggtgacggagacctggttcaaccggcagtcgccgcggtcgatgaggtccttgagacgtcccggagtggcgacgacgacctcggcgccaccacgcagcgccgacgcctgcctgccgatcgacatcccgcccaccaccgtggccagccgcagcttcacagagcgggcgtacggggtgagcgcgtcggtgacctgctgcgccagctcacgtgtcggtacgaggaccagccccagcggctgccgaggctcggcccgccggccggccgtacgggccagcagagccaggccgaaggcgagggtctttccggaaccggtgcgcccgcggcccatgatgtcgcggccggcgagggagttcggcagggtcgcggcctggatcgggaacggcacggtcaccccttgttggccgagcgcggccagcagttccccgggcatgtcgagatcggcgaagccctccgcagcgggaagcgcgggggtgatcgtccgggggagggcgaactccccctgaacggcgccgggccggcggccgtaaccgccggagcggctgggtccggccggccggcgcggcgccggcgaaccgaagcggctgccgccctttccggagtcggcaccgccatgacgggtgcgagcgaagcggtcgttcgtgcgtgtgcggttcatacggaaccttcctcgatgcggcacatatcaaggaatttccgaagcaatgagcagcacggagaatcgcaagaatggaccggtgggccttgccagcggatctggccgacagaaaatctgtgcggcacgtgcgctggaatgattgggggtgctgtgggctcgatattcgaagcgtccactgcactgtagctatgaaggatgcggctgcaccttcgaaggacgatccgtgtgcggtaaacacacgctgtccggagcgtcgtccgcaggtgaaatcactgcgggaaacgcatgtagctggggcccgcaccccgaaggatgcgggccccagctacaagtacgtgacagtcggcgtcaggcgggaacgatgttctcggccgtcgggcccttctggccctgcgcgatgtcgaagttcaccttctggccttcgagcagctcgcggaagccctgggcggcgatgttcgagtagtgggcgaacacatcagcgccgccaccgtcctgctcgatgaagccgaagcccttttccgcgttgaaccacttcacggtaccagcagccatgtcatttctccttcggggcagtcgtacgggatccgcaccgcgcggacctcgtgtcgccgcaatgatcaccccgcccggaaaaagaccggagatgtaaaagtgcttccaggggtactgagcccgaccggagcacttgaaatttcgggaaccacaactgcaactgacatcgacagtagcacgccacagcagccactgtgcggtgaagaacgccaccttgcttattgcggcagagaatctatccgcatgctccgatgaaaactcaaaccgcgcgcacagatattgaccttcgcgcgacgccatatatcgcatgccgcgctcgcgtgatccggtcccccaccacgctctccgctactgcacgggtcgcaccgccgcgggggcagacaggtccggccatgacgccggccatgctcggggcgtagcggacgcctgccggtcgggtgtacgtctcgcgcgcggcgagcactgcgggggaggggccggttgccagacgtcttgcctggcaaccggctgtcggctcgggctggttggtcagccgtggcaggtgatgtggttctgcgcgcccgcttccgtgaacgcgccgcagccccggctgccttctaccaggccgaccctcaggaggcgtgacccggggaagccgaggatcagcggtagtcgtcaggggaggcttccttgccgccgtaggtgacgtcctcgaagtatgcccaggcatccggccggctgccgtccacgtccgtcaccccgtatgccctggccagttccccgctggaggtggacttgccgttccaccgcttcgcgcggtctgggtcggcggccagcgccgcgaccgtacgggccaggtagtgcggggactccgcgatcgcgaacgtcggctcttgggcgatcgcgtcacgccagttctcctcactcacaccgaagtgggagagcatctgctccgaacgcaggaagcccggggacaccgcgaccgccgtgccctcgtactccgccagctcctgagccagcccgaacgcgaggcggatcggggcgttcttcgccaggtcgtagtagatgttctcgcggtagcggcggttggagtgcgcggtaccgtcggtgacttccacatgcagcggcgcgtcggagcggatcagcagcggaagcagcagcgccgccgtgatcacgtgcgagcgcgcgcccagctccaggatccgcaggccgtcggcgagcggtgtctcccagctcttcttcccgaacaccgaggtggccagaaggtgctcgccgccccacaggtcgttgacgagaatgtcgagccgctcgtactcccggtcgatccgctcgacgagggcgcggacctgggcttcgtcgagatggtcggtgggaactgcgattccggtgccgcccgctgcggtgacgagttcggcggtctcctcgatggtctcggtcgtccggccgacctcgctggcccgggcccgggtggttcggccggtcacatacacggtagcgccggcccgccccagttccacagcctgagctcgtcccgccccgcgggtagcgcccgccacgagggcgatccgtcctgccagcggacccttcggaccggcctgctcggtgttctcagtggtctgcctggtgatgtcctcgttgctcatgtcatccatcgttcacgctaaaaccgacagaacacgtcaccttttatgtggggggtaccgcgcatcatcccggccatagcgccaactacgtcctcgcactgagcgttttcagcgtgggccaccgatcgggtgacgccggtcaggtcggggtaggggccgcaacgcacaaggctcgcgtgcacgacatggccaccgcgcgcatgatctcccagcgggagcccagccgtccccggcagccccagccgctgagaccagctcacccgggacacccggtccgacaccgcacacgatcaagtagtcgacctccagacgcgttcagcagcccacatcccaggagccgtctaccgtcccaggaacccctgctccgggaccatcgggctcggcaccgggagtgcacagttgatcagtaactggcaacgagctcgtgcacggtaagcggtgaggtgtcgaggtccagatgggcggcggcggtggtgcccccagcggtcggccgaccggcatgccgagcgggcagcccaccggtgtgccgagcggcggacccggcggcggcacgggcatgggcggcacccccaccccgcagcacctgaagtcggtcaggaccggccgcgtgacgggcttcgggtcagacctgtgcggggaacagcaggcagtcgtccgggcggatgatcaggttgatctcgccgtccgtgtgccggacggggctctcggcatggacgcgcacgtcgccgatgctgagctcgtactcgaatcgcgctccggtgtacgagcactgctcgatcctcgcccggagcacgttgacggcaccgtcgtgcggggcgtcggcgcggtcggtgagcgtgatgcgttccgagcgcaggcccacggtggcggacgaccccgcggagcaggcgccggccaccctcaagcgctgaccggtctcacccagttcgacctgtacggctccgccctcggtggcgccgacgcgcccctccaggaggttgcagcggccgatgaagccggcgacctcgggagtggcgggagtctcgtagatctcggtcggtgtgcccacctgctggaggtgtccgtgcatgaacacggcgatgcggtcggacagggacatggcctcgacctggtcgtgggtgacgtacacggtggtgatgccgacctcccgctggaggtccttgagccagacgcgggcctggtcgcgcatcttcgcgtccaggttggagagcggttcgtccaggagcagcacgccgggggagtagacgatgcctcgggcgagggcgacgcgctgctgctgtccgccggagagctggtgggggtagcggtcgcgcaggtgagccatgtcgaccttggtgaggacgtcgtcgatgaggcgccgttgctcgcccttggtgaccttgcggagcttcagcggcagtgcgaggttgtcggcgacggtcatgtgtggccagagcgcgtacgactggaagaccaggccgagattgcggccttcggggggcaccgtgctgcgccgggtgccgtcgaagaagacctggtcgccgacacggatggtgcccgagtcgggggtctccagacccgcgacgcacgacaaggtggtggacttgccgcagcccgacgggccgagcagagtgaagaactccccgtccgcgacggtgaagttgacgtcctccaggaccgcggtcccgtggaaggacttcttgatgttctcgacgaccagctcaggcatgcttcttccccttcaggaggagaccggcgaggccggcgacgacggcggtgacggcgatctggagggtggcgagggcggccacggagccggtctcaccctgggtccacagatcgatggcggtggtgccgatgacctgtgactcggctccggcgaggaacatggcgggggcgtactcgcggatcatctgggtccagatgagcaggaacgaggcgagcatcgcgggcacgaggagacggagcatgatccgggacaccgtgcgccaccagtcggcgccggcgacgcgtgcggcgttgtcgagttcggctccgagctgcatggtcgccggggagatcgcgccgtacgccgacgggagtgcccggatgccgaaggcgatgatcagcgcgaagagcgtgccgcgcaccgcgtcgccgccgggtatccaggtgaaggcccagaacaggccgatgccgacgatcaggcccgggaccgcgtgcggtgactgcgctgtcgtctccaggagacgggcgaagcggaagtcggagcggcgtgccacgaggacgaccaccgtgccgaacagggtcacggccaccgcccccacgaaggccacggtgatgctgttgacgatcgactcggtgtagggggcgtagtcgaagatcagacggaagttgtccagggtgagcaggtcgaacgggttcaccagcggagtgagcagcgaggtgaacgcgcgcaggatgagcgcgagcatcggcagcagtgcgccgaagacgacgtacagaccgacgaaggcgaagcccagccacttccaggcaccgatgtcgagcaggtcggagcgggtcgccttgccgcgcaccgacacgaaccgctgggcgtgccccagcagccgcgtctggaacacgaccagggcgatggtggtgagcagcatgaaggtggacgccgcgcccagcaggccgtagtccggattgatcgagtcgatgccctgctcgtagaggaagttggagaagagggtgatgccggcgggctcgcccaggatgagcgggatggacagggtctcgatcgccgtgccgaagatcagcagacccgcgtagagcatcggcgggcgcagcatcggcaccacgaccgagcgcaggacgcgcagaggccccgcgccgacgctgcgggccgcgttctccagagaggtgtcggaggcggccagcgcgttggcgcagaacaggtaggcgatggggacctgggcgacggcctcgacgaacgccataccgggcagtgagtacaggttccagggcacccagccgaagccctcgcgcaccgcgccggtcaggaagccggccgggccgtagacgacgatccacccgaaggccaggacgagcggggagatgtagatgggccagcgcagcacctgcccgaacaggcgggcggcggggaagcgggtgcgctccagcagaatcgccatcggcaccgcgatggcgagcgcgaacacggtcgtcaggacggcgaagaggagggtgtcgaggacgatcgaaccgaagcccgccgacgtgaacaggtgggtgtagttcgagagggtgaaggcgccgccggccgcgtacaggggctggttgcggaccgactggtagaggatcggtacgacgggggcgaggacgagcacggcggtgacgaggaacgtcagccagtggatggtgacctcacgtccggcgccgaacaggcgccggtactggggcgtgcccagctcgcccgcgcgcgggatgcgggacggcgcgggtggcgccgggggtgtctggatggccatgacgactccgtacgaacggggtggggacaggggcgttgggcgggcgggggcggctcagccggccgccttctcccagcgcgcgacgtacgcctcccgcacgcgctccggcacccgcacgggccggtacagatggacgcggtccgcgccgagcctgcgccgcatgtcctgcagactgtccatggcgtcctggcgcacgtccggccggtacggcaccaggccgccctcggcgaccgccgcctgcccttcggcggagagcaggaagtccaggaagagacgggccgcgttcgggtgcggggcggtcttcacgacggacagcgcgcgcggcatgacgacggtgccctccgcgtagtagctccaccccagcagtcccccgctgtgctcggcggcgggtatcgcgacgccatcacgtacggcaggatcccgccgtgccgcaggtaggccagttcctggcgcgagtggaggcgcagcctgagacggaccgtggcgcggggtgcgtcgggccggacgagggacagggcgacggggttcgtgccgacgcacaggtcggcgaggccgtcgaaggtgaattcctcctctccggtgaaggcgtgggccgatgcggtgtcgccctcctcgaactccaggggcagtacacccatgccgatcaggttgttgcggtggatgcgctcgaaggactcggctatcaccgcccgcactcccagcagcgcctgtgccttggcggcccagtcgcggctggagccggcgccgtagttgcggcccgcgaccacgacgagatcgtggcccgcggcgcggtaggtcgccgcggcttcgtggacgggccccatccgcagttgcgtgccccgatggccgccctgcgcatggacgatgcggccgggtcccttcgagggcctggccgccgggtcgttcctgacccgaggcgccgatcaggacgacgccgtgcaacggacgcgaggacagcgtcagcttcgtccgcggcaacagcgacgaccccggtgacttccgatgacgcgcacgcccccgccgcccgaacccgagctgaccgtcgaccgcgccgcctgctctgggtcaccctcccgctccgcctgcgagatcagatcgccgacgcgccgccgggcaccgtcgtccacgtcgtcgccaccgacccccgcggcaccgctcgacctgcccacctggtgccacatgacaggtcacacctgtctcggcacgcccccggcgaacggccggtgtacgccccgaagctcaccgccgacgcgcgcgccacccgcccggacgcaccctggcacccgctccggcggcggcaggagcagccccggaaccggtgacgcatctcgtcggccggccgtttcgagtggaccgcggacgcggaacgtcacggcgtccggaaaccccggaaggtgaccggcctgcgtgtcttgaagccgagccgttcgtacaaggcgatcgcgccggtgttcgcctcggccacgtgcaggaagggacgatcaccgcgcgccgagatgcgctcggtgagagcgcggacgaggcgggcggcataaccctgcccgcgcgcctcgggagcggcgcagacggcgctgatctcggtccagcccggaggacgcaggcgttccccggccatcgccaccagggtgccgtcgacccggacacccaggtaggtgccgagttcatgggtacggggccagaacggccccggctcggtccgcgcggcgagatccagcatctcaggcacgctgtccgcgcccagctcgaccacgtcggtgtcggacgcggagcgagttcggccggggcggccgtcgccgggccaggtcatctgacggccctcaagactgaaaaccggctcccaacccggcggcggaacggccggggagctgaacatgtcggcgaaggcgccgggaccgagtaggccggccaggtcggcccagtcctccgcgtccgggtcgacggacacggaggagaaggtcgccacgtcggtgagataggtggctgctcgaccgaaccgtcgggcgagatgagcgtgccgaccactgagcgactgacctaccgggtcgtcgagtgcggggtcgtcgtcgttcatcatcgtgccgtttccttcctggtgagcgcggtggtcgaagggtggccgcggtaggcgaaaagtcggcggcggggcccgtggcccgatagtcgtagcccttgtcaccgtgcagtttgccgggtcgcctgaggacttccggctggaggccaatgccaaagcgctctcgtgccggcggaggcacgccttctgacgtgcctccaccggcaccactcagttcaggcagattgagcttgagcgatgcagcgccgccggaagtcgagcgcctcaatgcatcgagcggcgacttcctcgttctgggtgagagcagtcctgccttgtcgagtgatgcggcgttcggaccgtcaccccggcgaaggccaggacctgtcccacggagtggctcatccacctccccctcctcggcccacagcttcaggcccgacgtagggggaggggcgactcggaacccggcgtcccgctcgcgaaggtcggtcagacctgttcgaagtggaacgccttgatgaagcagtcccggggttgggcgacggcgaagaggatgcactccacgagggactgcgcggtgagggcgtcctcggcttcgcgtgaagtggtcgcccactcctcggagagcgggtcggcgttgtcgaagtcgggcgggtagagcgagatcacccggactccttgggcgcgcaggcgcttggagaggatttcggtgaaccctgcctgggcgctcttggccgcgtagaaggcgtcgtgtgcgtccgagcggtggtggcccggtgttccgcaggcggagaccatcgtcacgacgtcgggtgtgtccgagttgagcaggagggggaggaaactcctcgtggtcaggaccgtgccggtggctccggaggcgatggtgtccacgacgtcggcgtcggttgccgacagcaggtccggcccggtgaggtagcgggagccgttgttgacgagtacgtcgacgcggtcggtgtgttccgcgacgccggaggcgaagtcgcggatcgaggcaggatccgtcaggtcgcaggcgaaggcgtgcacccgctggtgtccgcggtcgcggatctcgtcgcggacccgttgggcggcggcgagccggcgtgccgagaggaagacctccgcgccgaggtccgcgaggcggatggccagggttcgtccgaagtcccggccggcggccgtgatgacgacgcggtggttgtcccatctcatggtgtcgttccccagtcgccgtttcgtggatcgggtggtgccgtgcaccgcgtctctacgctatcggtcatggtcgctcacgaacggtcgttcacggtcaatgatgatgttgaggtgcccaaccccggtgcggacgaggtctggaccgtcggcgcggtcatcctcaatcgggaaggtcgtgcctttgcccagaagcggagccgggaccgtcgcctgttccccggggcctgggacatcgtgggcggtcatgtcgaggagggcgagacgcttctggaggccctcgcgcgtgaagtcgaggaggagaccggctggcgcctgacccgtgtgcggcggttcctcggcaccacgacctggacgggggacgacggcggcggcctgcgtcacgaggccgactacctggtcgaggtggacggcgacctggaccacccgaggctggaatggtccaagcactccgcctacgactggttcggccccggcgatctcacccgcctcaaggagaaccgcggaccaggggagtacctgatccacgacctcatagccggtgccgttgccgactcgcctttcgacttgctccgggcggacgccctcaccagcccggaccggctgcgcgagctctacccgcagccgaacccgaactcgctgcgcaaggagaccgaccgcctgaccgaggagacccgggcgctgatcggctgttcgtcactggtgttcatcggcagcgcggaccgcgagggccgggcggacgtgacgccacgtggcggcccggccgggttcgtctcggtgctggacgagcagaccctggtgatccccgacgcgaccggcaacaaacggctcgacaccctgcacaacgtgctggagaccggacgcctggggctgctcttcctcgtccccggccgcccgaccacgctgcggatcaacggacgcgcctgtgtttcggcccgcccggagctgctcgcccgcctcactcccgtcggaaagccgccggtcaccgcgctggtggtgcaggtcgagcaggtgtatccgcactgcccgaagtcactgatgcgcgccgacgcctggcgacccgagcagtggatgcccgccgacgcccagccgagcagcgccgaggtgacccttgcgcagctgaacctgcccggcctgaccctggaccggatcgaggatgccgaacgggagtcgctgcgcctgcggtacgaatgacgacgagtcgatgagcgccgatgagccgatgagacccgacgggatccgacgggtcggcgtccgcggcgagcagaccggtcgcgaaggtcaccgcccgcacggcggcgaccctcgcgacggtcagtactgtccggtcaggtgcgggtccagcgttggttgctgccgttggagcaggtgtacagctggatcagggtgccgttggccgtgccgttcccgacggcgtcgaggcagaggccggactggacgccgacgacggacccgtcggagttgaggcgccacttctggttgtcgccgccccagcagctgtagatctggaccttggagccgttgccggtgcctgcggcgtccaggcacttgtcgccgtagaccctgagctcgcccgcgtcagtggcggcccactgctggttggtgccgctgtggcagtcccacagctggagctgggtgccgtcggaggtgctggcgtcgggcacgtcgaggcagcggcccgaaccgacgcccttgatctgtcccccgtccgcggggggctccgaggagtcgccgccgttgagtgcgtcgaggacggcggtgtacgcggccttcttgctgccgtcgttgttgaacagcaacggcgtctgctccgaccgccaggagtcgctgtcgcgcacaccccagacggtgatgccgaggcagcgcgagacggccaggcagtcgttggtcacgttggcgtaggtcgaggccggggcgccctggatgtccagctcggtgatggccacgtcgacgccgagggcggcgaagttctgcagtgtggtgcggaagttgctgttgtaggggctgccgctgttgaagtgcgactggaagccgacgcagtcgatcggcacgccgcgctgcttgaagtcccgcaccatgttgtacatggcctgggtcttggcccaggtccagttctcgacgttgtagtcgttgtagcagagcttggcggacgggtcggcggcgcgcgcggtgcggaaggcgacctcgatccagtcgttgccgctgcgttgcaggttggagtcccgccgcgctcccgaactgccgtcggcgaaggcctcgttcacgacgtcccactggacgatcttgcccttgtagtgggccatcacgccgttgatgtggtcgatcatcgcctggcgcagcgcgctgccgctgaggctctgcatccagccgggctgctgggagtgccaggccagggtgtggccgcgcacctgcttgccgttctgcaccgcccagttgtagacgcggtcggcggagctgaagttgaactggccccgctgcgg

SEQ ID NO: 31 - DNA, 3331 bp, Unknown
  Description of Unknown Organism:Unknown
tcggatctccccacacaacatagatagaggatatccgcctgggttcacaatgaagttactggtggttctcaccaccctcgtgggctttagctcagcactaagtttcggttgtaattacagaccagtattaggcttcaattcacagtatatgctgggaggactaagacttttctgtatgcctgccatggtttatgatccatgggcatgtggttgcgtttcggcatggagcagtgcaggtctttacggtgtcggagggggcggaggcgcctggggagctggcggtgctggaggagccgacggcggacgcggcggcggcggtggagattgggaatatgactatgatgacgacagcgatgacgatgatgaatgggactgggatgatgacggtggaatgggagctggcgccggaggtggtgctggtggtggtgccggaggtggtgctggtgctggtgctggagcaggcgcaggagcaggagcaggtgctggactcggacttggattgggcggaggtctcggaggtggacttggcggacttggaggtcttggcggacttggcggtggagacgatttatttgatttagatttcgatgatcttggtgcagctcttgccctcggtggagctggtggagctggaggtgctgctgctgctgctgcagctgccgctgctgccgccgggggtggagttggtggagctgctgccgcagccgcagccgctgctgccgctgcaggaggaggcgcaggtagacttggaggagctgctgctgcagccgcagccgctgctgccgctgcaggaggcgcaggtggacttggaggactcggtggcggacttggaggactcggtggcggacttggaggcctcggaggtcttggtggcctcggaggatatggaggatctgctgctgccgctgctgctgctgccgccgctgctgccggaggtggaggactcggtggtgttggtttctacggtggacgaggaggtagacgcggtcgaggaagaggaggccgcagacgtgctgctgctgccgctgctgcagctgccgccgcagccgctggtggtggcggaggaggtggaggtggtggaggaggaggcggaggcgctggtgctgccgctgccgctgcagccgctgctgcatctgcttcagcttctagacaaatgagtggtataagggacgcattaggagacattaaagaccttctcaggagtaatggagcctctgcaaaagcctctgctaaagcatcagcagtagcaagcacaaaatctcaaattgacgatttgaaggatgtcttaaaggatcttgcaggtctattgaaaagctcagcatctgcttcagcatctgcatctgcatcagcttcagctggaggtggaggcggtggtggtaacggaggtggtaacggaggaggaggcggcggtggagctggagctctagctgctgctctcgctgctgcaggagccggaggtggacttggaggtggaggcggaggcggagctttagccgctgcactagctgctgctggtgcaggtggaggaggttttggtggacttggaggactaggcggtcttggtgggggatctgccgcagctgctgcagccgctgccgctgctgcatcaggtggtggaggaagagcacttagaagggctttgagaagacaaatgcgtggaggtggatccgctgctgccgctgctgctgctgctgcagctgctgctggaggtggatggggaggtggaatgggtggaggattcggagtaggtctcggtggaggattcggaggaggatttggtggtggatcatcagcagcagctgctgccgctgctgcagccgccgctggatttggtggaggtggacgaagaggtagaggtagaggacgtggaggcgatggcgacggtaacggagctagtgctgtagctgcagccgccgccgctgctgctgctgctggaggatctgctgctgatgttgccgctgccgctgctgcagccgcagctatgtacggtgacggtgctgatggacctgatttcgataatggattcggtggtggaaacggaaatggaggtggcggatctggtggtggcggatccggcggaggtggatccggtggcggatctggaggtggcggtggatctggtggatcaggcggtggcggcggatctggtggttcaggcggtggcggatcaggcggcggtggaaacaatggatggggaaataacggcaacaataaatatgacgatgatgactgtgatgaatatggtaaccctattagaagggggtaaattatttgacattatccgccatttgactcatttttcttagttctctatgttttatacttcaccttagattgttttagtttgattgaataaattatgttttcgatataaattttttttaaattaaattaaactttattagttgacctgtaaactttttcatggagttataatctaaggaacaaaaaacatacataatatgttcagtattgtggtaaagcacctgtaccgcaaacacaatcacctctatacatgtatacaaaatcagtaatgctgacaaaatcttctacactctcacctacacactcgcacacagtcctcttacatacacagcactataatatcctgaacatgaagtttgtgttgataaaaagttcagaaaaatctcccctacatcacctgatctttcactgaaaatttacgacaagtattgaaaatagcagaaagaaaacgggaaattgagaagttttctataaaaaacaatcggaacaatgactggaatgacaaggatgaaaataatgataacttacattaattaaggccccaataatctctctattttcaaacttttttttcaaatgttctctctaactcacttgcatctatgtggaaattcacatactatactaaattaccacaagtatcaaggtttcacaacctctcatgccttcatggcagaccatgctgggtatttgtctaacaatgcctcataaatacataaaactaactaacaaaataggtcagtctgtaacaaattattaatgcaccattattgcattttctaaaacaaagcatacactggatattggcagacaaaatgttgttattggatacctttccattctatctagacacttgctttccacaagtcatcataaataaatcccccctatcccaaatgtcaatggaatgccccaacccttcccccataattttaaaacctagaataaattaaaacatctatagttcgtcatgatcatctttcttatcatcctcttcttcttcctcctcctccttcttcttcttcctcctcctcaggttcttggctgcctgctccttccttgccaa

SEQ ID NO: 32 - DNA, 5224 bp, Unknown
  Description of Unknown Organism:Unknown
ggatcccctgctcgacgccggcggcccggtacacctccatcgtgcggacgttgttcccgcgcccgcgagggtggacggaggtgccggcgtgacgctccaccagcatgtgccgcaccccgagccggcccaggaacacggacgtcgacaggcccacgagcgatccgccgacgacgaggaccggaaccctgtggaccgtgtccccggcccgatcggctcttgcgttcatctttctcctccagcgcgtgatgtccgcccactcggccggtttcggccggggtcatgcatgccccgcgaggctggagcgcggtgcgccggggaccacacttcacccgcttaacccgctgcgttcgcgcaggggcacggcacgcccgacgatcgtgctcacgggccgacgcaccgtcatgtgacgcgtcggccgccttaccgttcctccaggaagaggtgcgcctcaatgacggtctctgccgctgtgtccacggtcccggaccgtgtccccctcaccgtgttcgacggttcccgggtgcgggtcgtgctgatgctggacatccgcgacgggacgcaagcggaggtcctggacgcctacgagcggatgtccgaccgggtcgccgccgtgccggggcacatcagcgaccagctgtgccagtcgctggagaaccccacccagtggctcatcaccagcgagtgggagagcgcaccggagttcctcgcctgggccaacagcgaggaacacctggagatggtccgtcccctggagccctacgtccgcggcacccactcgatgcgctactcggtgctgcgcgagacggccgaggagcgggccggggcgggtgcggcggcccggggcgcgctgcagccccggccgcgcatcggcgacaacgtggtccggcacgccgtcacctacaccgtcaagcccgacagcgtcaccgaggtcgtgaagatcctctccgcctacacctcgcccgaggtgcgcgtggacgacaccacgcggctcgtgcgcacctccctcttcctgtacggcaaccgggtcgtccgggcgatcgaggtgcggggcgacctgcaggccgccctgcgccacgtggcccggcagccggaggtgcgcgccgtcgaggaagccctcaccccgcacatcgaacaggaccgggacctcaccgacccgcggtccgcccggctgttcttcacccgggccgcgctgccggccgtccaccacgtggtgtccgggcgcgggacgggcggcgacacgcagcggtgcgcgctgtactacccggcccaccccggcgccggaccggcgctcgcccggctgctggcgcggcagggcgaggccaccgtgggcgacccgggcagtccggtcgtcgcctgcaccgtcttccaccgcgacgacctcgtcgtacggctcgtcgacacggcgggcgcaccggagcgcgcgcccggggccgtcctggccctgcacgagccggacgccctcgccgaggccgggcggctgctggacgccgccgcgctcggcgccgacggccccccggacgaccgggcgctgccgacgttcctcgcgcacgcccggatgcggcctctgacagaccgtcagtcgccggcctcctgaccccccgctcgcccgacctcagggagtgaccgacatgacagaacagcaggcacgcatcgtcgccttcgacgacgtcccgcccaaccggcggcgcggcggcgacgtccgggccctgctcacgcccacgaccgcgggggcgaccagcggcttcatgggcgtggccgtcgtacggcccggagaacgcatctccgagcactaccacccgtactccgaggagttcgtgtacgtcaccgccggcgccttcgaggtggacctggacgacgtgccgcatcccctgcgcaccgggcagggcctgctcatccccaaggacgtgcgccaccgcttccgcaacaccggcgacgtcgaggcgcgcctcgtcttccacctgggtccgctggccccccggccggacctcgggcacgtcgacaccgaggagaccgacgagaccgcgccggccggggtggtgtcatgagccgccgggtcgtcgtcaccggcataggcgtcgtcgccccgggcggcatcggcgcggcccggttctgggacctgctggccggcgggcgtacggcgacgcgccggatctccctgttcgacccggcgcgcctgcgctcgcagatcgccgccgagtgcgacttcgacccgtccgcgcacggcctggacgacgagacggtccggcggtgcgaccggtacgtgcagttcgcgctggtcgccaccgccgaggcggtccgcgacgcgggcctggacaccacgcgcgaggacccctggcgcatgggggccgtcctcggcacggcggtcggcggcaccacccgcctggagcacgactacgtcctggtcagcgagggcggctcgcgctgggacgtggaccaccggcgggccgagccgcacctgcaccgcgccttcgcccccagcacgctcgcctccaccgtcgccgagaccttcggcgcgcagggcccggtgcagaccgtctccaccggctgcacgtccgggctggacgcggtggggtacgcctaccacgccatcgccgagggccgtgccgacgtgtgcctggcgggcgcctcggactcgccgatatcgccgatcaccatggcgtgcttcgacgccatcaaggcgacctcgcccagcaacgacgacccggagcacgcctcccgccccttcgacgcccgccgcaacgggttcgtgatgggcgagggcggcgcggtgctcgtgctggaggagctggagcacgcccgggcccgcggcgcggacgtctactgcgagctcgccggctacgccaccttcggcaacgcccaccacatgaccgggctcacccgggagggcctggagatggcgcgggccatcgacaccgcgctggacatggcccgcctggacggcacggacatcgactacgtcaacgcgcacggctccggcacccagcagaacgaccggcacgagaccgcggcggtcaagcggtcgctgggcgagcacgcgtaccggaccccgatgagctcgatcaagtcgatggtgggccactcgctcggcgcgatcggctcgatcgaggtcgtcgcctgcgtcctcgccctggcgcaccaggtggtgccgcccacggccaactacgagacaccggaccccgagtgcgacctggactacgtgccgcgcgaggcacgcgagcgggagctgcgcagcgtgctgtcggtgggcagcggcttcggcggcttccagtccgcggtcgtgctgaccggaccggagaggaggctgagatgagcgcaccccggcgagccgtcgtcaccggactcggagtggtggcaccccacggcatcggtgccgagacgttctggaagacggccgtggacggcaccagcagcctggcccggatcgaccgggagggctgcggccacctgcccctgaagatcgccggccaggtccccgacttcgacccggccgccctgatcgaggacacctacctcgtccagaccgaccgcttcacccacttcgcgatggcggccacccagctcgccctcgacgacgcccggctctcccgcgccgacatcgactcgccgtactcggtgggcgtggtgacggccgcgggctccggcggcggcgagttcggccagcgcgagctgcagaaactgtggggccagggctcgaagtacgtcggcccctaccagtcgatcgcctggttctacgcggcgagcaccggccagatctccatccgcggcggcttcaagggcccctgcggcgtggtggccgccgacgaggccggcggcctggacgccctcgcgcacgccgcgctggcggtacggcgcggcaccgccaccgtcgtcgccggcgcgaccgaggccccgctggccccgtactcgatggtctgccagctgggttacccggagctcagccgcagcgccgacccgggccgggcctaccgtcccttcacctccgccgcctgcgggttcgtgcccgccgagggcggggcgatgttcgtcctggaggaggagggcgcggcacgcgagcgcggcgccgacgcgcgggcgacggtggccggccacgcggccacgttcaccggcgcctcccgctgggaggagtccagggccggcctggcgcacgcgatcggcacggcgctggcgcgggccggctgccgtccgcaggacgtggacgtcgtgttcgccgacgccctcggcgtgccggaggccgaccgggccgaggccctggccctggccgacgcgctcggcccgcacgcgcggcgggtccccgtcaccgccccgaaggcgggcatcggccgggcgttctgcgcggccgcggtgctcgacgtggcgaccgcgctgctcgccatggagcacgagctgatcccgcccaccccccatgtgctcgacgtctgccacgacctggacctggtggtcggccgggcgcgtcccgcccggccgcgcaccgcgctggtgctcagccgcggactcatgggcaacaactcggcgctcgtcctgcgcaggggcgccgcgccgttccccgagtaagtaccccgaacaggtgtctcacgtccccttcgggcgcgggcacccgagtcaaggagctcaaccacatgaccgacatgaccgaacgcgtgggcacccaggtgaccttcgaggaactgtccgccctgatgaagcgcaccgcgggcgtgcacgtggaaccgcctgacctgcgggcgcgggccgaggagggcttcgacggcttcggcctggactccctgggcctgctgggcatcgtggccgagctggagaagaagcacggcgtgggactgccggagcaggtggagcgctgcaagacgcccgcggagttcctcgcgcaggtgaacgccaccctcaggacggcggtgtgacatggccgggcacaccgagaacgagatcgtcatcgccgcgccgctggacctggtctgggacatgaccaacgacgtcgagaactggccgcggctgttcagcgagtacgcctccgccgagatcctggagcgcgagggcgaccgcgtccgcttccggctcaccatgcacccggacgacgagggccgggtgtggagctgggtctccgaacgcgtcgccgaccgcgcctccctgacggtccgcgcccaccgcgtggagaccggccccttccagttcatggacatccagtgggtgtacgagcagacgcccgagggcgtgctgatgcgctggatcc

SEQ ID NO: 33 - DNA, 30601 bp, Unknown
  Description of Unknown Organism:Unknown
gatcttagaccttattcacttgatacgtgtaatagttattacgatagtatgtttttggccgattcctccgcgtcttctttcgacgacgtggaggtggaggcaaaagcgaagtagttgtggaagaataagaattatgattatcatgattattattcaaattaactctattgttacgtaccgcgctccatgcagacgtttgccaggagacgacgggtggaaggataggaagcgaagaagcggaagcggaagacgtcgtatttgaattcgaagatgataatgatgtcattgatgctgatgatgttttgttgtgataatgagatcggcatggaggcatttcacaatctctttgttcgcgaggcttaatctctgagcactcgatatcgtcttgtttacgaccggaaagcacgtcttcacaccagattttgcggcgttgaactcccccgccacacgatacagaacactggaattattattagaagcttcaatgatgttctaagaacttacgtgagtccatggagatatctgccaagaattatttgtgagtggtggacatagttcttcattgcatttatcaaacatctttggttttctggtttcatcgcaagaagacgaagtgcaagatacactgcgacgacgccatcccccaccacaagaagcagagcactgaacaatcgtacattagtaaattctaaatctgaaaattatatatcccacttttgaccaatctccaataatccaggatccaatatgttcttctccctttggacagggttcaagtcggcaatttcttgcacttgttggtctcttttgcacatcacaatcaacatctttcaaaatcgtccgaccaccgtcttccgcactgacgcatgtaacatttctactttgttgaacatgagttccacaagtagctggacactcttcccattccgccattttccagtatgaacaatcacgaaggcgacaatttcttgttgatacttccttatccaaatgattgcaatactcatcaggaagatcacgaacatgatctcggcacttgagaagacgacgttgagtaccattaccacaagttgctgaacaggctgtccatggtccggttgcccatcggattggtggtacgtcggcttgaagtttttgaagtactctgggcccatcacaagtatctttttcacaagtcttttttaggcgtggacgagtattctgaaatgatattttcgttcaagattaaaagcaaactgaaacgtactcgatcacaaaaatattcatcaacaatagttccttcagatccacgagtacacgaaacacttctagtctgaattcctgatccacaagtgactgagcaaggggaccaatgacttggtttccaagatgtacatggcagaagatggcaagtttgactagtttctggcattttggtatctccacaaaaagaagcatcaacagattgttcgcggtatatgcattcggttgtacgttcccgatgaccgattccacaagatacagaacactgaaacgtatttatggtattgacaacagcaattctggagtatttgaataaacttacagcactccaatcagtatttctccaaaatgggcaagtgcctaaattacatgtctgatgagaagcaggccgatcagatgcagtaccacaaagtgacatatcgacttcagttccatttccagaaacacatgaaactcttcttgacgaccatccatcctcacaagaaacactacactgagaccattctccaagtttatattttggacatgattctctatgacatggttttgtaattatcttttccattttaaggcaacgatgttccggtagtactgatctatgacgatcggtacaattagcgtctcgatactgtacaccatctccacacttagctgagcagtcagaccagactccgaactgccaccaagtacaagcatgttcattacaatgttcttgtgtctgtgctggaccacatctggatgtatgtgtttcccgatcggctgcatccaaacattgagcatgacgcattttgactccaccatcacaacttcgagagcactctgaccaatgcccataaacccatcttggacatggaattctgttacattcccgttctgtcgcctctttctgttctctgccgcacaatgactcatcaactcgacgattcgaatcatcaacgcaatatgacttccgatgcatttttccattcgatccgcaagtttcagaacatgaagtccattctccatagttccattttcttccagagcagtcaatgtaacaactggcaatatcggatggttttgaattacgatcacatagatgttcggatgctggagtttgacgatcaccctccatttttacgcaagaaactcgttgacgtttctgtccagatccacatttggcactacaactagacacatcttcagtgatccatctgtaaatataaaaatttattatagaaatctaatgaaaatatgtagtttaccttgtagaacaatctatattgcacattcgtgttgcttgttttggtttgagaacattttgacaatttctatcatgactttgacgatgagtcgacatgtccagacacattaatttttgcgattgctgtccacgacaggctctatcacattctgtccaagtatccgtaactctccacaaatacaatgcactggatattggccgaattacagcatttggaacagccgcagtcatgtactcatatgagatgtcgggtggatgactaccaacagaaagaacatgaacataaatgtcacttctaatcggaccagttccatttatccgttcaataattgcatcagaaccagaatattcgagaacagtgtcttggaatgcaatttgttggcgagccagtgatacttggaaatgaccgttaagtaggaattcaccattggcggcacggagagctgaaggttaaaataaagattttcatgggtattgataacacaagggtgagtgatgaaaaaagtaaatgttccaaaaacactttgtatagaaactcacaaagataattgtcatcttctttcatattattatatcctttctgccggatatcaatatttgcagaaccagctggaatcttcattacttcgttataaccaaaggttccttgctcattaaatgttcctttgacaaccttacaggaagaatcatccccaccgcaaacaccacatttgtctcttcggagagttgaatgaagttgatgatcacagcctgaaaatccacttattttcaattttcttttgaaatcagatcaatgttacctgctggcatacaagctccagctacacaaatatcgtctccatttctatcacatggtgttccatcaacaactttatctcgaagcagatagaacgctgcagatccactgagccgacaatacagcttgcaacgttcatttggtgcaacattcgcatattttggaacccagtgagtattcgttgaagcgacaccttggattccaatatctttattgttgaattcagaacattgaacttcacggtatggttgagtatcccatgggcattcttgtgtattacatgaccgataacgttctcgttgaccaacacagtactttccaccatttcgaggtctaaagtaatatgggaaaatgtcattttaatattgataggaaagcttagccagtgtggcctaaaagctggaagtttttttaaagatgcgttttctatcaatttaagataaccggctacttcaggtgattctataaattttataaagcttggaagctaggtaaatctgaaaagccttaaactatctcgaagcggcccgaaagcccagaaaagcagagacggacaaacatttaagagtgatcagaagcactccataccttgatgttacatttgattttagtgtttccacctcgttttcacttctgaactcgccgattgaaaatattttgattgaatatattatttgctttcagactatttgatatcatttcgtttggcagtttaactcactttgggctgtcacaatctcttaatcctttttgaacaccaccaccacaagtacgactgcattctccccatgatcgccagtcaccccattgtccgtcaattttggtaagggattcgggggctagacgaacacaggctccatgatgacagaactaaatatccaagtttttatgagtttcttttgtgattaatttctgagatactcaccatgcttcttgattcgtcacaaggagttccgtcggcccatggcatatgctgagttcgacagcccatctggcttccgtagaatgttgcacaccaaagacggcggcatgtcggctgtaaaatatcaatgtttcatcttaaagaatatatttaggcaaactaaccatataagggcacaactcagaagctggtccaaatacaaacttgcactgttgatgagcatcgtatttctttcctggttcatcacgtacaaagacatcctcgtagtaacgacgttcgaccggctgatcgaatagacattgagtttgacctcgattatttctgacaaattgacaattaaatagaatcaaaattttaatagctatcttactcgaggaatcgttcgagcattccagctgaacatggcgaccaactccatggatgagtgttatattccaacgttggtgccattatgtggaagttgttctgaaactgcgttttatcaaatttagtgctttggaacttgcaaaccttattaaccggcatgtaggtagagcattttcgttcgtcatcatgaggaatcgaaaacacatgacccaattcatgagcaattgtgaatgcagcactcaatccattgtcttctatgattgcacaacttttttgcatatcacacattgttccaagttcagcaagtccaagtgtatcgcattttccttgtgatcgacaaatatctttacgcgtcaaaaggattgcaacgtcatgatgttggacactcgaatcatctggatcattgtaatactgctgccatctacagaaatcttgaagtgtttgttgagcgttctgagtgattcgtggtccagcgttttccgttttcaaaacgatcaacttgacaacaacgacattgatagatgcacgaagggattggtgacgatagatggaggcaactgtggagaagagagtgagaacgtagtcttcaagagatcttccgtgatattcgtacatttttgtatccgccaccacaaggacttcaacatagtgatcccaagagttggcagctcttcgggatcttgctttgcgttctataattaaatccttttgtttcataaaattatttaaacatttttttactgtatccttcctattaatcttgcaccccagagctccactttgacctatctttgttgtcattgactctatcaaaaactgttcaactatgaaaatggggatgcaagactaataaaaggtattggtaactggttccagtagagctttttttactatctgtttcattgattcaattttcagatgtttatataaccatcttaaccgttcaaatctcataacatagaacagcctggcagcccgtgaaaaggtgctgaaatcccagtaatttcaatggcattcgaccacacacaagtgatccattatcctttgctcttttacttcgttaactaccattagctataggggacccacgagcaaaattctatagtttctgtgtgtgttagggtgttttaatgggctattacacaacacccgatgggatcagcagaatctgagatcttttgggaaccggaaaaaaatattgtgataacttctcttttttctacattttttacagaactagcaggtaaactttcagattgaaatctcgaaaaatgcatccgcctactcaaaaagtcgtttttaaaatgattgtttctttgtgtttgtcctctttttcccggacgtacgcaacacaaaaccgcttgcgcgaggatgtacacaaaacgtacgttctgcgcaatcttttccctgcagctctctctctctcactttttctactccataaatcagttctctgtctgtctcccaccacctaaatcatcatcagcatcatcacagtccccccaccaagttcttgtgtcttctctgacctttacacgtcgactagggaaaagctctcaagcagacactcgagcgccagttgaaaaaaatagtgtgtccaaatgagcagtttcgaatttgaaccgtttgttcttgttctgacataaacccaaaaaaacgaactaggcggcaaaagagatctggataatctaaagaatctagacaaatttcagaagttcttaccaataacatcttcccactgatcttgccacgtggcaaccgtcgtctccgtctcgttgacactggtcgagttaagatggtcaaacgatttgaagtgcattggatcgaactttcggacgagatgttgcctatggcgacttgctccgtcgtgctctagatgtttaaagtgtcagagaaagtgattacaaagtttctacctgttccgtttccactaataattggctcaaccgtatggattccgctgggtagtgcaagcattccgtactgaaaaaggcttttattcaccaaaattcgaacttatacaaaccaatccgtcttccgagtcgcataaattgacgatgctgtgctgatgtacacctttaacgtgtgcacggtagatacaatcgggatctgttcgagacattccacctctaacctcctcctccgagtccaaatataagaccatcggcgcgaaatttgaattggaaaagtggggaacacttctggaattgaaaattaatacgactgttataataaaattgaaatctcatacttgttatgtgagtccggtatttgattccatctgtgcaaatgaacgatgtagacggcatcatctgatcgtaatcgtaagtgacaagcatgtccacagtctctggcaactccttggagtcgacgtcgccgatctgttgacgtgacatcacgttttccacgacgtccataagaatctcttcggacgatgtgatggctgtcgatgacgtgtataccggcgtcttgacgccatcgactgtgatgcactggcacacctgaaacttagaacattatttcacttcaaaactttttggattgttacctgagtacttggccctggagaacagcacatctgatgagaattctgagatcgtgccactccctgctgaaaggaacatttggttaaaaacaaaagctgataaattaaaataattagataaaaacgaacattgcaacgcataaacgaggcagacgacgaggagtatgagagcggcgacgacgggctgcagcagatggaatgagccgccgatggagcgcataccaacagctccgtgatgatgattatgattgtgtggagagcagcaaagaaaaaagagatggaaagaagcagaagctccgataaagttcgtccgtctcttctgaaaccttccaaaaactacctgctcgaggtgaagggaagtcgtctgattgaactgctactgcttctgatcttttgataatctcccgagtttgtgttttcgtttagtcgaattaaaattgtagattgtggaatgagcacttgcaatagggaacagagcatcacagactgaaaaattaaaaattatctagaatgcaagcaatttttaaatttgttttaaaatcacttattctgacgccatcttcttttccgatttgcgcagaataaataaaaacttgactgtaatattgggaaaatttcgaaaaaaaacaccgttaagtctgagcccacctttcgcctttttttgttgacgaaaaaaaccaaacaagctttaaattcataaaattcccaattttaaaaacatctaaagtcaattcctcccaataatgcatttgtatatgaacaaaagtctgttgaccataagtcgttatattactacaagcaattggtcatcaacaaacctcataaaaatcagttttgaacgggagcaatttatataaactctgtgtgctcttttgctctttttcttatttcttagttgtcttctagttccgccaccactttcgctgctcttgacgaaatctgtaaattgttcgtcatttttgatttataagatttgtttggctctcggtaggagctctcaagctgctaatagtcctatagtaaagtactaaaaacacaaagaagcagatgaaggtgtcataaaacactgataagaatcatcatgattaggttggtgcagagaaaagaagaagaagaaaaaggagatttagagaagagaaacaagaataaaaatgcaaaaataaaaaaaatagtaataacaatgaacgcagagtcttccatgttggagaaggaacaggacccatgttgatgtgtatctgaggggatccaatgtgtagtgatggtagtaaacacttgagagggaacttccacccccgactagatgattggaagcaattgatgatagatgtagagccaaagaattgggacctactaatgatctagtcaagattcttctgataagagaaaaagacaaggaagaacatgaaaatgactggtgattgaaaaataaaacggtttatgaagtcggggtgtactaaagatgcaaggtctcttgtgacgtattttttcttccaggcacgttcgcgttattcacgattttatgcaaacaaggtaaggagtgttttgaattttgaatataaaaatttaaaagaaattaaagttagacatttgaaaaattagacaccctcatgggaaaaattatagggcgaggagaggcggtgagaggcgccctaatttctgctcggtcgggtagaatgtctaatctaaatcctacctcatgtttggctccttcttaaatcaaaagcttaaggtcatctctgaaacgtgcagttgacaagttcaatggtaagaacagggagcaagcatttacaacaaaaaagtaaacaaaaattgcatttgtcgcagttcaaaatggaacaactcactcccactcgagaacgttttgaaggggagaggaagaagaggaaaatcatcacacaggcacatggaacttctgggacacaaaacaatacaaactgggtgccgtgaatctcagtacacacacacaaaaatcaaaaaagacggaaattaggagcagatgtggtaaagggtggttcaatgctgatgggagagagagggagaaacttcaaaaaaagaagtttagatttatgttggctatttcaatcctaaatttatctaaacaattctaaaaatgctggttttggaaggttatctggtaatggtgaagttttataaacaaaacaagacaaacaattcttgagatcttaaaaatcttagcgactacaacaatatttaggtattttttaatggaaaaaagtattgattgttgacttgggaaattgaacagcaattttttgtacttttaaatcagttatattttaactttttagagcacatttcgtagacaaaagggaaaacgattggtccaacatgtgaagatgatgatgtcaacaagttttggatcggagccaaaaaagaaacaaaacattcataccatgatgggaaacaagaggtgcagcaacaacttttatcaatattttgtttatgttttgattatttttctggcacccagccagtaattcttttccgtagagttgacctagaaaatgttggaggcggagtcttaggatcaagagacgcagactatcaaagtaaaatgagtaaaaggaagtgatataaacttaggaaacggaggaaaaaaggacgatgataagagattgaagacttggaagagtgtgctctttgcgggagagcatattcttttgagaaaaatgggacctaggggcaactgacgcaattgaaacatggtcgagcggtcggcgggaagacaaaaagtgaagaaggatgggcaagaagaagcaagagaaatggcacccaccgtggaacatgatcatgatgattgagagtgaaaattggaaatctcgaacttttttgcaacggcgcgttttggaaaactaacaaagttgaccaaaaaattattttacatgtataccgggatgtctaagaattgtaaaattgagtgatcctttctgtgacataatttaaagcaatttattttggttatttctaagcgcctttttatactagcatgttatattgttaattttattatctaaactgccgttcttcctatatttattattgcaccccctttgttcattctgacagactatacctcgattaatcataaaaatgtcacaaaagaataaaaacaactaaaattaagaaaatacaagaaatttatcaattgccaaaaattcggccaatcggaaaaatgcttggttgccaatttgtcaaaaatttagtcaattggaatttgtcgattttccgaaatgatatgaaagtttgaatgatgcagctaattttgcagtttaagtttacattttcaagtttactgtaatttttccaaaatatgaagaagagttttacgaaattaaaagataataaaaaagcaatgcaaacatagctatgaaatctgatcccgactaagtttgatggacataggattaataatattagtctaactttctatagaacactaaataaatacattcactctcgaaactctcccttttctgccatcaactaccgtactcacttttgactcaatgacccgcaactgtcaagatgagttagtttcaagattctctgaaacagcaataatctaacaagagaaactgaaaaaatagagtaaaactaataataataccacataaattgacatgcatgatagatgattttccggttttcaacaagaaaaacaacaatttccgagaaatcctcatagtttttggtaagaaaaaataaattgatagtgatacggtatgactattacttctaaagacttacctgattagaaacgtgtagtaattgaagaagaaaagttgaatttgagaagttgaatcgagtttacgatgtctgaaaaaaacatagatattatggtaagatcaagcatagaaaaaatggaaaaatacaagaaaatagagactagagattgcataggttttgcggtggcgaaaccgcacacatttttgtctgtgttatctctaattttacgctctcggtgttctctatttactgtccagaagaatgaagaatatgggggaaaagtgcgcgggaaaattgagagaccgagtgatgagagccgcagttttgcaaaactttttcgggcaataatccgccggcgagtactacgagaagcacacacacatacgaaaactgttgagttaaaacctaaaaaattgtttcgacatatttaattttcgaactaaagtttagagggtctgtgcgtgcatttttgaattttccaaacaactttcagttttgcggaagaaaattacagcgattttttcgaatatttctgaaaacaacactattgcgtatcaaaaatttttcgatttgccaaaattcagactaagttttggtggttttggtttgcaaacatttaaaagaactcaaaaaacatttttagatgttcgaaaccgtacaattgtaggatacaaatagctacagaacaattagaatataaaatagagttgtcaaacatgtttaactaatacaaaaacacagaaactttgaaactcgaaatttttatatcaaaattgaaaaagcttgtaaaatttaaatatggatacagtacaaacaatataatcatagatcaaatagttcatttatttatatatcttggcaaatcaaatcgtatcccttacccactcatattcgatgagtctacaattaaatcagttgttttttcatcctcccggactattagtttaacttccacttgaacaagggcaaagagtacattaggaagagtttatgatgacaggaaaaaagctatgtaaaatgacctctttggattgaaaaagcgaacgaattgaggtttaggacccccggaaaatgaagaattcgtggcctcgagaatagcaaattggcggaattaattatccgtaagagtgtgaattggaaacaaccgggacgaatggattactgaatcaaaaatgaaagaaagaagagatgaaaatacgtgtgaatcggatgaaatgtgatgattttagaataacctaaatgcaacaaaacgacgtaaagacgcggaagaacaggaatgatcaaggggtacatcttataggggaaaaatgcactttttgtgctccaaatgtgagagataatcaggtaggaagagacgtagaataggaacaggaaacggtaacgatagtgcgcaggtgcttgatttctgtgcttttgcatgtgttccgatggaatttttggaacttttcaaggggtttcggaaagggttcgagatttcgcatgtgagctttggaagaattttggaagaactttcaggataacatcgctcaagcttgtttgttagatttcagacttcaaagtatataccgattattgaaacattttaatcgtttcttactattagtaaagtttaatcacagtttgaaaaaaaaatcacaattttttcaattatttagaccaaactaattatggtacagaaaataacttgcaacccgggtatttcattctaatttttttcatttggaaccactagtttttgaaatagaaactcgttaggattcttcacatattatcataactatcagtattttgttgcacatcagatctaagttcagtctaattagaatcgcaaatttgaccatcacactttaaaacaaatttacttaggcacagggcatccttctaacttttttgtccccgacaaaatgatgacaaaaatgacgtgaggaatcaaggagaaaaaggaaaagaacaggaagcgaaaagtaggagaagctcttgatttctgtgctcattccttgttcggatgagctcactgtttgcaacattggcgttggtgcgcgggaatcgccattgccgaactttttcaagagacagagagagagagaaagagaaggaaaacgttccgatttttaaaatggaaaaaaatgaaagaggaagatgatgaaaaaatgaactctgcgtgacatttgttaatatggaaaaagcatgattacttcaaaattgtacactaatccccacagcacacattttgaagacttttttacaaaaacaatggtttaagcaagctttaaaaaattgatagtatccttaatgcttaatcatatccaagtttagttttaagttttgatttcaaaaatttctacatcaaaaaatcatacttagtgattatatgcaaaacaatttttaaattcaaggacatatttttgatttttggaaggatgataacttttttgtgattccgaaaaagattaaagtaggtttaaaacctctgaccttctacagaaaaaacattacctctatgaattttttttcatctcgttcagaacttgtctcgggtcaagccatgaagacatgagatagggtgtaaaacgttccgaagagaggtttatgactattattgtagttgaagagaaaaatgatatctcaatggatttcatacagatggtcggatttcattcataaaatatcataagaaaaggtacgtttatgactgtctaggtcaactggttttaggtttcttggaattgtttcaaacatttttaggaaatattttcttgcaaatatctactaaattgaagtttgttattgtttttgacatattgtagattttagagaagaatcactcagagcaaaaatgttgggaaaacgtgagaaaaatccaagagacaaaagaatggtcttactattagtagatcaaaaaaccagaccaattattcatattcctactattcaatatatattcaaaaatgagcaaaccaagaaattgcacctaatttatcatcccacatatattccgacgaaacattcgctctaccttctttttttctgtctaggaattataaagggccataattataatttcagtcaaggttttggaaaattgttcgactaaccattatgaaagttaaaaaccaatcagtcaaaacacacaataggaatataaaattcgtagaagaaaagctttttttttggtcgaaagcaaaatcaaattctggaactgcgacttttttagtgcaattatccattcaacgcaagttgtctttcaaaatttaaattccagaagagttataacaaaacagacaggtgtacaagtaaaagaaaaatacaagttttatcgtaaaaactgatacgaatctagatacacctgttaaaaaaggctttctcgaaacccagatgccgtacgaagtaagcagcagccaactaaacattttgagtaaacatatggcaagtgttttggcgcaaattgtaaagattttccgtgtgggtaactagaatttgaaactgtaagtatgacgacttaaccacacaaaatcaaatttcaaaagatcttaaaatgttcgaactttcaaaacttttaagctctctcgcatctaccgtagtcttctaataacaacagtcgtaagagaaagctcaaaatttttcaaactttttctgaatgacagaatcagttgtatacaaaaaaaacccccaaaatgcgagccccatgaacctgacaaccagacaagtcgaaattgtaaaatcgtatagatcttggttcacgacatgaagagcaccgcgggggcacacgagagcaactactgcaagcgctcctgaagagaagaaacatcttttttccaggaccactggccagtagtgctcccccagatcactttcttttttcttgcttcatctgatttgtgtctgcgtcgtctgatctctttagaacctatccttcttcttcttctttttgatacttcgacatcagaacaacatcgacatgtatcatcttttctcttttttttttgttatctattcattcattcacttttcatttagtttgattaataggtgacatgaactcttgtcacttttcaatttcaacttcttaaatcttaaactcacagtgattccagatatgagcaactccaatgaggtgttgagtagaaacctaaatataacattttggatgttttgataatgttggaacaaataaattgaaacaaacaagacttgaaatagagacaacgtgcagaataatgtctaccagctggtttcagtggcatattgtaccacgaacgtccgacagaacgaataacataaagatcaagaaaaactgtttgggagcagacaaacaatcagaacacagttttgttgaggggaccaaatcataattaatgactaaattttaacgaagaaagtgctcgaaaagaacagaatttagaagttgatgaacaatatttttacttttagattaacaattatgctttacaaatgacatccaatctaaagcatctggtaatctgaaatttgtcaaaacagctttcaagactagtttcaaatttgtcgattcaatggatcaagtgtgtaattgatccaataaaaaagagtataaagtgagaaggaagaaagtgtgaaaaaagaagaacgtgaaacgtgcagaagatacgaaatgagtttgaagactgcacttttcgagcctcgatggtcagtcacttggtcagttgcgaaaaagctgtgaaaatgatacattgtgtcggctctcgtagagaagaaagccacatggtcaggatgactccaactgggatattcagttgtaaagaacacaattgatatttttgcatcttttttaactagtttttacaatatgagaaattgttctgtgcgaaaaatatgacttcttccttgttgccgaagtgtatttccctggaaattccagtaaatacctaatgtaaaaaatctcagcagaatgtgttcttacattttgttgtaataataatgtattaaaattgcattaattaaaaatttcttcaaaatgttcctacgtcttctatgcacattatttaggtcacagtttcatggagcacaaaacacctgccgacgcctctaaaatagttataactgcgcatgaaatcaggtagaaaaaactacaaaataaccaatacaaattgagtagggcgatggagaggtgggcggttggagaggcgggcaacaagcgtcctcatgacgccttgttcatttagaatgtgtttgctttgaattacatacaagtttctaaaatttaacttacaaaatttaaaaaaagtcacaacaataataaaagttgtggcaatgaaatgttttaaaaatctaaatattgagttttaaataaatgatttttgaaaattcacaaagaaatgttacaatctgtgaatgaagacgaacaatgaaaaagtgaggaacggacgcggatattacacattcagtcacacaataaacgttcggacactaccacacatttctctcatcatttttttccaaagtttattctaaagttcaatattttagtttgattattttggacactattcttaaaattaatgtataatagtttagaaaatattttgaaacatgaaacttttttgttgataaaatagtgccaaacatccttatgttacgcagttatccaaccacatttttctcatttttccaccaaaaaacactgaaatggtccataaaacctattcaaatggatatgagaatattacttttttgacatgaaattttcaatgatgtaatgtaaaacaaagaaaaatattgcgggaaaaattgaacggcgtattgcaaaaatcggtgtgcggaggaggagaaggaaaaggaagagcaggagaagcggaccgaagaattcagaagcttttaaaataagaacggcgactttcagacaaacaatggactgttgtataaaaataaagcggaggcggtagagagtcaaagctttcagaaatgtattagaataggtttcactacctgttgttgaactcaaaaaggtgtgaaaaagtgaaagtttgtctgaagtttatgacgggaagtgtccatcaaataactttcaaaatttgacttatcagtgagaaaaacacgtcattttggaacgttaaaatgggtggcaccgcaaaatgttcacaatgtgaagtgaattacgtaataaaatcagttttattaagcttattaaactaacccttccggactatttgtggaatgaaacaattgggggggtttttttttccaattttcgatttttttttgaatttataattaccggaacaaaaatatctttaaattattaagatttgagtgatgtttgaaattttgaacctgcaaaacataagcacaaaataatggagtttttgttttaaaatatcaataggtgttttttcacagaactttaaacaacaaatactcataatttgaatgaaaacagtagatcccacaatattttgaaaacttatctatatatatatatatatatatataattacgaaaaaaaaacaaaaagaaaaaaacaaataatttgtcagttgataatttttagatatgagttgccaaaattgggcaatatggtgaagaaatacggtagttcgtcgcactgtcagactaattttcaagtgttcctagtggaatgaaactaacagaagctatacggtatataatattaggaacacaattaaaacgaacagcggaagaaaagatctagtggtcacttccgatttctcagctgacttttgaatgggcacctatcatcatctcacttgtttatttgaacagtctcgactttttccaattgttggcttctagttcaagaaacgaaaaaaagagcaataacggaacagaaaattcagaaagtggaagagaaatatgagaaaatgatgatgataataataataagttagaagagggttatcgatgaggaacggaaacgttatctctgatcgccatctcattattattatgagacacaaagatgtaagttatggtatctttgaaagaaaagaaaacaggaaattatacagaacacacacaatttcggagatttcattcgaagaacctaacccaatttgaactcactcccacttcctcttgtctataaaacagtcaatcacaggaacaggtgtctgtcttttcaaaatgtatacgttttccgaataatgacacacaatatcacagacaaaatgatcaatgaggttgcagaaaagaatgcaaaaaaatatagaaagagagggtgaacaggagatagagaatcaaaatttgcatagataaatatgcaatagaaaataacaatttttgaacaacaaagaaataatttagtggcatataatatagcgatggaacttgcaaatttttagaattatcatataaaaataacaatgtttctatattttatgccctataagtcttgcagtatttcttaaatttaacagttcatttcttggtaatctttatttttatcaagaagtgttcaggaaattttaggacatcaaatttttatttattttctaaatctacttttatcaaaattttagaggtctagtacacatctacccaaaaagaagactttggagctctcaaaaaccacctagtgtatggtaaagtacatgagaagtgacgtgtctttgggcagctggccatctttgtcgatatgcgggtgatggtgtttctgtgagcagtaacaggaaattctggacacctgctagggtgtcaaaccaaatttatttcaacccattcttgcttcaaaaaacccccaactaaattattcaaattctcgtaatttaatgaatcactcagtaactgtaacgttttttttttcagagacaatgatcgaaagttaacaaaaaaaactgaggattaaacgttatttggtatctacagctgacattggaacatatcaaaaagtggtaagtgaaagtgaaacgaaaagtgcaacatttgaaattgagagtagaaaagatcattgaagcagaaatatggaagtgaattgaaagccgtggcgccaaaacgacggtcaggcgccattgagaaaattaatgagagttcggaaggttgaaacaacacaaagacaacgtgaaaaattagtttggagaagataaaaaatgtctggagatggacgatttcttagttagctgagaatagtttacattgattttcgggaaaacgcagaatgttagaaaaatggaaacatgtctagacttcagataaatttgtagaatttatatttgtagcaaaagcacactaacaaaggttacaaagctattaggaaaaatacggaatgtatttttgaaaatttttgatttctctaaaataataacaccattaatttgctatatttgctatatatgctatatagtatgttcgcattactgagcacaaaacttggaaaaagtttaaaaaaaaaggaaacttgttttctggagaaatcattaaaaacagtacaatttcagacagaaataaatctttcagtgaaagcttttttttgagtaagactaagtatgcactcacaacttttctgagtgttccaaaaatgtttaaagaaaatactagtaaaaatgagcatttcgaaaagcaatatatcatacaactacacaaacatttcaattaaaggaatcaattttataatagttctaggcaatcccacttttagattcaattttctagcacagggagcattggaagatataaaaacataaagataaaggtgataaaagatccattaaacacatcatatctatcaaaccatcacttccatcaaatccacagatttatcacaaatcagtgtgtgacaaatataccgtaatattaagttcaaatggtggaaaagacgcagacaaagcttttgcataaatactaaataattgaaagaaacgcagagaatgtaagagaaaaatatacaatatgtgtattatcaaccatcaacagtttttgattaaaaccatggagaagcgatatacaggagcaaattaggagacgcagattgagaaaaaatgagaaaataatgaaagtacggaagggttattgtacaataagacaggtagcatctctcaaagaacctattgtcaagcagtttaaacattcaacaacgttcatttattttttagccttcattatgatatctcattggttctataattggattttttaaattcagatttctcattcatgtacaagtaaagttgttaattggttattatgcccaaagtttaattatttgagcgcagaaaatttgaatggaaatttcagaaaactgattcatgctaacttcaaaaaatcctgaataaataccaattcttttccaagtatgattctcgagcctgtttacgtgcctgcctacggtctattttctaatttttttaatgataaaattttagagtagatcttcaaaaatcttccttaaaaaatctccaaaaaaatcaagttcaggaaaactaaagtactccaataaaatactcttatgcaaaaaccccccattcattttgcagaaaaagacaaacaagaattaaagataaaaagttatgatagacaggaagctgatttattagatcaatgaatcgacttttagtttttcttgaactctaatttgaaatagtattcgaatgagaaaattgaaaatatacaaagatcaaaagttataattgaaaatcaacaaattgatagtgtttgtataggattaaattaaaatgtgcggtacatgagacagtagtagtagtagccatagtacgtattggtggctccactcggctactgataatttccttttttactgataatttgatgtcatttcgtaattttatttgtgtttccaaaaattgtgggcgtggtttatgaattggtcaagacatgaattaaaggaattgtaaagtaaagaagaaaatgacagaggagaaattattttcgtttgctttggaaattgcaaaataaattagattattaaagataatagttacggtttaaaataaataggtgataaaaaaatatccaaaagttcaagtcctaagaatcttgctattttgcaaaaaaaaagcatgagcttttggcctaaaaatggcggacagctgtcgggacactatccaagaattcgtgataaacgggtgaagcaccgtctcttatcatcatgccatttttcgaattttaaactcagactttgataaagaaaattaaaaagagagagtgtgagaaataagagtacacatggaaaatgcaagatttgaatttgtttccaatttttaaaatgtatttaaaagagttaccgttccatttttgattagctttataagtggaaaaatcgtttttggattattttttgaggaatatttttgaatgcgctttcaattttcctataaaaaactttgtgttcacttttttatcccgtttttatttttatttttacaactttcaaatttttatgaatgttttattgtaaaatcataaaaaggtgcgaaacatctaaattgcctggattgcatttaaaagtgcattagcagaaatgtattcctatggaatgttttttgtgcaacgagatccagaagctcgaaaaacatccaaatttcttccaagaaagttgatgttccaaaaataaaaaagattttagcccaatcaactaaaaaaaaactctcgtttttttcatatttcacattttctggtcactttgaaggaaacactaatcccaaactgagaaccgaacatggattaaaccatcccatttactatttcttgttgtcttcaaaaagtcttagaattgtgcaaaaaatagaatgtttcgaaatattgcggttttcgttaaaaccttttttgagtagattgagggtccattagaattcccaagagaacttgatgaccttcatcatcaaaattagtggtcattgaatgtttgatcagacaaaaatggaaatgactgaatcggaaagagcaagaaaatcgaaaaaaaaagtatttggaaattctggaaaactttttaaaatttaagaagggcaacgataagaaacaggaaattagggattttttagtgatggagaagtacgtgataaggttaaggtggaacactagtgcacacgttttgaatacactacgtgtttttatttatggtagaatatagcacttaaagaacgtttttaatacaaactgaaataaaaatacggaaatgtaatttttttttttgaaagaatccgcctgaaactgaattttcacatcaaacggtagtgattctctttatgcgttgggtgatatgtatttacgctgtcttaaagttttcgactataatttaagtaatatgtttgtcaaaaatcatcatggtgctgtgtcctatgtagccttttctacacttgaaaaatgataatttttatttgaaaatggtatttaaattcaagtagaaagttatttagtcttgtgtgccaagcaataaacacatagtctattaggcaataaaaagtcagctactgtttgatttaaaaacttagactactggtgtgcctgtgcaagttactcccgtagtacggatacagagtgaaaactagtgattgtactttagatcggctgatagtgaatttacagagaaataattataaaacttaaaatttttagcagctcagtcttcaggctgcacagccatattgttacacttggagttacaaattctgcaaaccatctaggattgaatgcaaaaactctgaaagtcacatcaagaaattccaacaaaaaacacattagatgccaactcattgaattgcattgattcccaagagaaatagtagtaaaagtgacccctatccattcctccgttacatacaaatatacacacaaaaaagagtgtagacctcttccttctaacccaaccaacacacaacaatatcgttcccttttatctctaattctctgcgtctccataagctttgagagctcttcggagcatcttgtgcttgctccttgtacggcggtacagtttcctccctctgctcccttatgtgtgtttaggtgttgtttgaacaaataagtttttggccatccacctccttctcaaaacctttttcttatgcttcttcttgttttgtgcacattttggctcttgcttgtctgctcgagccatagacaaggcggcgacatttttgaaaaaattatattagtactgttatatagtacttaatacaacgatcacaacaacaacacaacgaaatgaaaacatgagatcaaaagacaaattgttaggaggagttggagtttctacaatcatgaaatgtttatctagttattataaaactgaaattgctcataaaattgtgataccatgaagaccgaaaaactctatgcaactgcatactgcacatacttacaacctttattctgacttgaatttcagtttttggtgtttgcagttattctattttgtttaaaagaaaattcaattaggaaataagcaataaattttggcatgtatttcgatagaaggcacgtgtaaatgccacccggaaattagaaaaaataagatttctcaaactgaaaatgattgtgaattgaaaatttaagagaatcattgcaaaagtacacaaatgaatcatttttcagattgaacaggaaagtgcagaaatatcagattaccgtcccaacagaaaccggaaataacacttttcaggtaaagaattatacagaaatcgtaataaatttaaaacaaaagagagttatgacacattgcagaacggtctctgtggaaaataggaggaggtgctgcaaaaactccttagacatggtcatacttacaaaaaaaacagagtttaactaaaaattaaattaagtgagaaaatgaagaaaatggaggtctttcgcggattcattttacttcttcttttttccacttttcgttgcaagctttggtttaaaagtttcgcaaacaaataaacaatgaacattgtgttgagaagacaagccaagtgaaaggaaaccattgagagcaaaaacaacaatcaattgaaataaagagtaaagtttattgaatatactgatatgtgaatactggaaaaataattagtctctataattggtaccgcctggaagattcatttctgattcccttgtgtctttgaccaaaactttatttttttcagttcaaaattacaaaaaataaatactcatcttcatcgattcagtggtgttttaaactcctacgtttttcttttacaataaaggtaatgtaaacgttccgagcgtgtagttttctctgaaaattttttaaaaataacaactttatggtatttttcttaaagtcttaaactgaaaccgaaacatttttgataggaaaactattttaacattttgggaactcggcaaaagctctgcaggcttgccgaacaactctcatttgaaagtaataaatatgaaaataaattatcgaagtttttttttttgatattttatgaatacggctcttggtagtttttgacgagaaaattacatgttgcataaatttcaagagttataactcatggagaccctaatttctggtttcactagaaaatcaaaaaatcaagcgtttgagcagaagactgtaggaagagcacacgtcataaaaattaggggatcaacgatccgaaacggggaattgaaatacgatatgcgatgagttttggttcgaaccggctttgtcccaaaaaacaacagaacgatggtctcaggctcacttgactcatctcggtgggaacaatttttatttgtttttattccgtacgcacagaaactttttttgaggtatttttgatcgtgggtgggtggaatggtagcacccaatttcaaatagtgtttgatttgaagagacaatgaaagaaacaagtgggagataatggaaatgacgtgatgaaatggaacggaggaaaactggtataaatatcgttgactatcaaaactacaataatactaatggagaaaagttcaggattcttgaagattttacattatgatagttgggatttactggtttcaagttcaaatgtcaaacatctggaagaaaaacgtataagattacatcaaaataaaactaaaatttgaaggataaagtaaaacagcataatatagtgttttacatctcatgtaggaaacgaacaaaatctttgaacacctagataacttcaaacggaagttgggtgaagaaaagaataggggccagaatagaaggtcattttgacaaagtgaacagacaaagacattcctaactcggaggtattccaaaaactgttccaatattgaagaatgacactatttgattttatatcataacattattaatcacatggcttttttcttaggaaatttatatcgcaaaataaaaagtggccttgatgagtcattcattcaaaacatgcctaaaaaccttcataattaattataaaaatgctgatacttgaggacccgtttttttatatttataaacagttgttttctttattccgttctcactttgagtttttttctgaaaatactaaaaaaattaacaaagttcggcgttttttgtcgataattccatctgattattttcggtttttttacctaattatcaaatattttagccagagtgaaatttattatcttattaatatgtttttcaatttgttttggtattattctgttgaaggaacatgttgcattttaaatctgttgttaatacagcggccacatgtttagaactttataacctcgtttaaacataaattgtatgccatatttattgcaagtactacatgagtttgaaacagtatcagatactatattttaaacaaaaatacacattttccccgctatgagagattctgatacattggtttccaatttttttaaaaacttgaaattcctcaagtctcccactgaattacagatttctgttctagatacctccaaagacacctagattcgacttcggcatcttcctcatttttatcttcagtttcatcttttgtctaattttccgtacatttctttgcatccttaccatctctccctctctcactcactcttcttgttcactaaatctcaattcaaaatgttttctgccacgtcatcatcatcatcaatgccaccttctcagagcccattcgaaaaattaccacggcatcaaaatattcgatatcacgaaaaatgcttctcaattccacttcatacacttaactattttctatgcgttattattttttatttctttgttttcactatattttatcacgaacgttatggtggaaaacctgaaaatgttcaagttacatcagcaatttatgattcaaattcaaacgaactgtcattaatctttctatttgattcttcaattcgtcgacgggaaatattccttggatttggtccaaatgactcaaaaacatcaagaaatgaaactcaaattgagcttaaaccaccacccggatttgttgataactcacaaatttcagtaagtttaggattttttttcaaaaaaacttgatatgaagtgttgaaaaattgataattgggccgggcttacatcagagtatctagttatcttgtatttcaaatattaatattcaaacattgtagagattcgaaatgcgacagtacttcagtaattaccacccacattttgactgtcaaaaaagttcccaaaaattgtcgaaaacttttattaggatgttttctcattttggcacgattggagtgtttttttaacaaatcccttttatgcatcaaattaatatctaatttttaaatcaataatttggattaattcaacttgttttataagattttctcgctattaaattagcaaaaaaaaactatcttcaaacaattagcgtgctttaaaactactaggcctttgttggcaacgtcttttcacattttggcacaaaactataaactatgctcagaatttggtaatgtttgaaaatgttttgggcaagcatatagttattccaattctaaagtaagattagtcatctattttccattccatttttccatttttcacctattttttccattatttaacaaccaagactgagcaaacattttcctgttttaattttcatatatgaaaagacataagcaaaagctggatcaaagcttgggcaaatcctattcaaagtattttccaacgtttccattccctcgtttgtaaagtacaattggtaatcttaaggcttaattaattattgtgggagattcataatgtgaaaactaaatgttaagatttggtcatcaattgaaaaggaaaaaccccagtctttaactgtgaatgcagaacatccaaagtcattgcttttacgagatcacacaggacatccatatttagaagtaagttcaaatcagaaatccccaatccattttttcttgtagttaccacttcaagaaccatactccgattttcgcgacattgttagttgtttcagtccaatttatggagattttgagatggttttaacaggtttaacaaattaatttggtttcttttttaaaacatttaatttttatagctttaacatcatccatatcaatgggatcatttgttagtataccatatgaagagcttactggagagctttacaagtttctacgtgtatttgaaaaaacgggacatgtcaggttaactgcatttccaatgatacgtcatcagcctcgcttcgattcggaaaatgaaaattatcatttgaaaatgatcaaacttaaaacagatttaacgcatttgcattgttggctaatgcataaaaaccgggccaaattcatgatcttccaaaactctgctgaaattgttttaccgatttcctcgacgctggaaaatcccaattacgcctctgaatttacacgaatatttgaaacaccacgagttgaaggatatgatattttagaatataatgtcaaaatttcaacggataaacgcttaggcgacttttcggatttctccatcaggcagacaattgaagcagcaaaagcagaagaattaaccggaaattctaaaacattaatcatgagaatggtatcactttttttcaaaataatttactgtttctattttggcatttatttcagcattctccaactccacagaatctcttaaaacgcggtaaaatgtatccatttttcaaaaatttcccatctccaccacaagttattccaaagaaaacattggacaaattggatacaataacagaaataattgaagaatctgatgcattctggacacttatcaaagaatgttcagaaaattcgaaatcttggaaatgctcgtcaagaaaatgtgtaagaccatcagttagacatcgatctcttcatggatggtattcatatgatattcatttttctaaatttttgaatgttgaaagttttttttgttcagattttcaataaacttttaagaaaagaataattttaaattctataattcctgaatttccaactatgtttatcatttcccaaagtacattcgaaaaagctcaataagcaaaacgaccacgaaataacagtattaaaaaaaaagatgttgtcatttgaagttctggagtgcgatgaaaagtctctcacctcggactttctgtaatttatttagcatacaacatgaatttgaccaactcgaaataaggttaagactgaaaatttttcacaaaaattggaacacttgcgaagcgaattcaagacttttcgaagttattaaacaagctttcaaattctcagtaaaactgaacgttttttttatgctctccaaatcattttaatatggctgctcgcgtcgctgaagtattttctagagtatgtttaataaaactaatatgtaaatgaaaaaccaaaaactcagataaagagcataacttttataacgcattttcagaactcttcaagctttttcagatcacttctatcagcagtattcttcttttttccaaagacaccaagaactgaaaaggttgaaggagcatcaccggaaatagaggatgactgcttattgttcttctttttctgaataaaatcaaattaaacaccgaaaatatgaaacatattcactaacctgaacagctttcaggtttgatttattctgattttccgccgctgatctgctctgacttttgaaaccgggacttggagagttaccattgcgtatgcgagttcgaactggacgccgattcttctttctgaataaacgaattatacaaatttgtatttgaaaacggacaacatacactccttcttccgccgaattgctcatcgattttctcatttcttgtgttttttcctggcgttcaggttcaaaaggtggagcaactggtttggacatatacggaagaatgttcgagacttgaatcttttttggttgctcaatattctccattggaatatgatcgggaagttcaaagtagctgttggatcctggagcttgatcaaatccttcgagagttaaaagttcacgaactgcttcactcattgtgaccctttcctcttcggcaccagcacagattctatactgaaattgcttgttgtgttgttttactcaaaagaatagtgaacaaaattttctcaccgtaatgaatctgacaatggctggtgggacgttagcttcaaatggcattcggtatccgttctgaacacgtggtaaaacctcagcaactttcattcccggataaggttcgattccatcatggtacacttcccaacacatgactccataagcgaaaacatcagtctttggagtatagaacccagttcttggaacttctggagccaaccatctaataggaactctgaaaaatttgaaaaggttggaatttttgacgttctctaactttttgtgaggattcatccgatagctatagccttctcgtgacagtccaaagtcggatatctttacttgtccattcccgtagagacaatttctggacgcaatatcgcgatgaattatttgaagtgaatgaagatattcaagaccaagaccagcttgaagaaccatcgtatgtttcttggaaattggcaatgaaccaatgttcttctttagatatgaatccaaagctccattgtcagcctaaaataatttacataagacattttttcttagtaaaataaaattaatcagttaattaattaacataccaactccattatgaccatcaaaggttcctgtcctgcagccacaccataaaaagtgacgacattcggatgtttgaactttctcatcaatctggcttcgtgcatgatttctttgatctgctcttttgtcaaagattccaactttgccagcttgattgcagcttttttgacggtatttcctatgcgaatttctcccaattgaacctctccaaatgctccttctcctaatttcttgattaatgtcacgtcagaatgttgcttttcccacggttcacgaccaattggacggatgattacagtttctgggccctaaaagcaaacaaatgaaaataagtttactcacttaatttgtaagatcaccccagcaacaggttctttagaacgatgatagtaattgagaagatctgcgatactagaaaaccattttttatcaactgcaaacttgttattgtgctctcgaattacataatgacgaatctgaaataatattcttaaaaattatgagcaatcgttttacgtacgtcctcaattactccaacatagacagagagaacaaatttccttggctctcccacttttggatcagtaaatcgaactagaaaatcgcctcgttgagtgagcaactgtttcatatcctcacgtggcaataagccatggtaccagggttcttttgcaagtacttgc

SEQ ID NO: 34 - DNA, 8009 bp, Unknown
  Description of Unknown Organism:Unknown
ggatccttggccacgccatgggcgatgaaattgaccgcgtcgtaacgggtcatgtcctgctcctgcaggaagaaggccgcgttcgattcccgttccgcaaagatcgcgacaaggacattcgcccccgtcacctcggtccggcccgagctttgcacatggatcgcggcgcgctggatcacccgctggaaggcggcggtcggcacggcttccgagccttcgacttcggtgatcagcgtcgagagatcatcgtcgatgaactcggtcagggtggtgcgcaactcgccaagatcgacgccgcaggcgcgcatcacgcggctggcgtcgggctcgtcgatcagcgcgacgagaagatgttcgagcgtcgccagttcatgtttgcgcgtgttggccagcgccagtgcggcgtgaattgcttgctcgagcgtggtcgaaaacgaaggcatgcggcgctcctttcctcgggtctcccgatactggcctcatgtgattaagtttcggtggatttcgccgcgcttcaaggcccggacgcgtgttttttccacctctcgccgctctgttgcaaaactgaccagcgcggcgggctttcgcgcgatccgcaggcagcgcgcgaaagtgctttcagaaccggtccttgcgcgcccgcgccgcggtgaagacggcgagcggcgcggcatccccgggcaggccgagcgcggcgcgcaacgcagcgtcatcggcgcgcaaaaagggattcgttgcccgttcctcgcccaaagtcaccggcaaactgggttccccggccagccgcaaggccgtcacccggtccatccggtcgtgcagccgaccgttccccggttccaggctgagcgcgaaccggccgttcgcggcggtgtattcatgccccgaacagacccgggtttcgggcggcagcgcggccagacgggtcagcgtgtcgaacatctgcgcgggggtcccctcgaagagacgcccgcagccccagctcatcaggctgtcgccggaaaagagcagccccgccccgggcagataccaggcgatatggccgagcgtatggccgtcggccgcgatcacctgcgcggcctccatccccagatgcagcacgtcgcccggggccaccggatgatcgagcggcggcagccggtgggcatcggccgcggcccccgccaccttggccccggtcgcctgcgccagcgcctcgacccccgcgatgtgatcggcgtggtgatgggtgatcaggatgtggtgcagctgccagcgccggtcggtcagcaccttcagcaccggggccgcctcggggacatcgaccaccaccacggtatcggtggcggtgtcgtgccagagccaggcgtaattgtcggtcaggcaggggatcggggtcagttcgagggtcatggccttttgcgcatctttcgctatcctgacccagcttcgcccaaggaaggccaacctgcaatgcatctcgacgtgctcgacctgcgtgatttctactaccgcacccaattggggcgcacggcgcaaaaggcgatccgcgacaaggtggtcgaactctggccggacacccagtccggcatggccgggctgacggtggcgggctacggcttcgcggtgccgctgttgcgcccctatctgggccgggcgcggcgggtgatcgggctgatgcccgcgcagcagggcgtgatgccctggcccgccggagagcccaatgtctcggtgctctgtgccgaaaccagctggccgctggagaccgggatgatcgaccggctggtggtgctgcacgggcttgaagtctccgacgaccccgatgcgctgatggaggaatgctggcgcacgctgggccccggcgggcgggcgctgttcatcgtgccgaaccgggtcgggctttgggcgccgcgcgaaaccacgcccttcggctttggccgcccctatacgatgggccagctcgaggcgcaggcacgacgggtggggtttgcccccgaacgtcaggcggcggcgctgtacattccgccctcgcagcggcggttctggctgcgctcctccgagatgtgggaacggctgggcacaagggcggcgggctatctggcggcgggggtggtgatgcttgaggtgatcaagcaggtgcattcggtgcgccgctcggggcttggcgcggcggtgcgcaagccgctctcgatccttgaaggggcgcccaagccggtggtcgggcggatgtgagccgcccgcggccgcaagaatcgcccggccggaaaagcccgtttccgcggcacttcgccctgcggcggggaaacgcagcggggcgggcttcgaccctttgcgctaacactccgtgccggtgcagaaaatgtgccagcctgatgcggattcctgccgccaagatggttgcgagggtcttgatgctctgctagacgcaaccccgaatgcggcgtgcgagatcattttgggcgccgaggggggcctctgaatcggtgacggaacgattggttccggtgtccgcgtgcggaggcaaaagcatcggaagggtggacgtgtccgaaccagcttcgatttccgcagccattgccgggcgttatgccacggccatcttcgacctcgcgcaggaggccaagggcatcgacgcgctctcggccgacgtggacgcgctgacggccgccttggccggttcggccgagctgcgtgacctgatttcctcgccggtctacacccgcgaggagcagggggacgcgatcgccgcggtggctgcgaagatgggcctgtcggcgccgcttgccaacggtctgaaactgatggcgacgaagcgccgtctgttcgcgctgccgcagctgctcaagggcctggccgccgcgatcgccgaagccaagggcgagatgaccgcggatgtcacctcggccaccgcgctgagcgcggcgcaggccgagaagctggcggcgacgctggcgaaacagacgggcaagaccgtcaaactgaacgtcgccgtcgatgaaagcctcatcggtggcatgatcgtcaagctgggttcgcgcatgatcgacaccacggtcaaagccaaactcgcttcccttcagaacgccatgaaagaggtcggataaatgggcatccaagcagctgagatttctgcgatcctcaaggagcagatcaagaacttcgggcaggatgcccaggtcgccgaagtgggccgcgtgctctcggtcggtgacgggatcgcgcgcgtgcacgggctcgacaacgtccaggcgggcgagatggtcgaattccccggcggcatccgcgggatggcgctgaaccttgaagtcgacaacgtcgggatcgtgatcttcgggtcggaccgcgacatcaaggaaggcgacaccgtcaagcgcaccaacgccatcgtggacgttccggcgggcgaaggcctgctgggccgcgtcgtggacggccttggcaacccgatcgacggcaagggcccgatcgtggcgaaagagcgtcgcatcgccgacgtcaaagccccgggcatcattccgcggaaatcggtgcatgagccgatggcgaccggcctcaagtcggtcgacgcgatgatcccgatcggccgcggccagcgcgagctgatcatcggcgaccgtcagaccggcaagaccgcgatcgcgctcgacaccattctgaaccagaagtcgtacaacgacgccaacccgggcaacaagctgcactgcttctatgtcgccatcgggcagaagcgctcgaccgtggcgcagctggtgaagaagctcgaagaagccggcgcgatggaatacaccaccgtcgtcgccgcgaccgcttcggacccggcgccgatgcagttccttgccccctattcggcgaccgcgatggcggaatacttccgcgacaacggcatgcacgcgctgatcatctatgatgacctctcgaagcaagccgtggcctatcgtcagatgtcgctgctgctgcgccgtccgccggggcgtgaagcctatccgggcgacgtgttctatctgcactcgcgcctgctggaacgttcggcgaaactgaacgaggatttcggttcgggctcgctgaccgcgctgccggtcatcgaaacccagggcggcgacgtgtcggccttcatcccgaccaacgtgatctcgatcaccgacggtcagatcttcctggaaaccgaactgttctaccagggcatccgcccggccgtgaacaccggtctctcggtgtcgcgcgtcggttcgtcggcccagaccaactcgatgaagtcggttgccggtccggtgaaactggagcttgcgcagtatcgcgaaatggccgcctttgcgcagttcggttccgaccttgacgccgcgacgcaaaagctgctgaaccgcggtgcccgtctgaccgagctgatgaaacagccgcaatattcgccgctgaccaacgccgaaatcgtggcggtgatctttgcgggcaccaacggcttcctcgatgccgttccggtgaaggaagtcggccggttcgagaaaggcctgctggcctatctgcgctcgacccgcaaggacgtgcttgagtggctcaccaaggaagaccccaagatcaagggcgacgccgagaagaagctcaaagacgcgatcgccgagttcgccaagaccttcgcttgacggcctgaaaggacagggagatgcccagccttaaggacctcaagaaccggatcgtgagtgtcaagaacactcgcaagatcacgaaagcgatgcagatggtcgcggcggcgaacattcgccgcgcccaggaaagcgccgaagctgcccggccctatgccgagcggatgaacgccgtgatgtcgagccttgccggtgcggtgggctcgaccgacggtgcgccgcgcctacttgcgggcacgggctccgacaaggtccatctcctcgtcatcatgacgggcgagcgcgggctttgcggcggcttcaacgccaatatcgcgaaactcgcgaaggcgaaggcgatggaactgctggcccagggcaagacggtgaagatcctcaccgtcggcaagaaaggtcgcgacgcgctgcgtcgtgatctgggccagtattacatcgatcacatcgacctgagcgacgtgaagaaactgagctacccggtggcgcagaagatttcgcaaaacatcatcgaccgcttcgaggcgggcgaatacgatgtggcgacgatcttcttctcggtcttccagagcgtgatcagccaggtgccgaccgccaagcaggtgatcccggcgcagttcgaaaccgatgcggcctcggcctcggcggtttacgactacgaaccgggcgatcaggaaatcctgaccgcgctgctgccgcgtgcggtggccacggcgatctttgccgcgctgctggaaaacaacgcgtccttcaacggggcgcagatgtcggccatggacaacgccacccgcaacgcgggtgacatgatcgatcgcttgaccatcgagtataaccgctcgcgtcaggccgccatcaccaaagagctcatcgaaatcatctcgggcgccgaggcgctctgacggaaccggagatagaagagaatggcaagcaaaggcaaagtgacccaggtcatcggcgccgtcgtcgacgtgcagttcgaagacggcctcccggcgattctgaacgcccttgaaaccaccaacaacggcaagcgcctcgttctcgaagtggcgcagcacctgggcgagaacaccgtccgcaccatcgcgatggacgcgaccgagggtctcgtgcgcggcgcggccgtgtccgacaccggcggcccgatcaccgttccggtgggcaacgccaccctgggccgcatcctgaacgtcatcggcgagccggtggacgaacgcggtgacgtgtcgaaagccgaagcccgggcgatccaccagcccgcgcccgatttcgcggcgcagtcgacggaaagccagatcctcgtcaccggcatcaaggtgatcgacctgctcgccccctattccaagggcggcaagatcggtctcttcggcggcgccggtgtgggcaagaccgttctgatcatggaactgatcaacaacatcgcgaaagtgcactcgggcttctcggtgttcgcgggcgttggcgaacggacccgtgagggcaacgacctttaccacgagatgatcgaatcgggcgttatcaacctcgagaagctcgaagaatcgaaagtggcgctggtctacggccagatgaacgaacccccgggggcccgtgcccgcgtggcgctgaccggcctgaccctggcggaacagttccgcgaccagtcgggcaccgacgtgctgttcttcgtcgacaacatcttccgcttcacccaggccggttcggaagtgtcggcgctccttggccgtatcccctcggccgtgggctaccagccgacgctggccaccgacatgggcgcgctgcaagaacgcatcacctcgaccaaagccggttcgatcacctcggttcaggccatctacgttccggccgacgaccttaccgacccggccccggccacgtcctttgcccacctcgacgccacgaccgttctgtcgcgtgcgatctcggaactcgggatctacccggccgtcgacccgctcgactccacctcgcggatccttgacccgcaagtcgtcggcgaagagcactatcaggtcgcccgtgacgtccaagggatgctgcaacgctacaagtcgctgcaggacatcatcgccatcctcggcatggacgaactgtcggaagaagacaagctgacggtggcccgcgcccggaagatccagcgcttcctgtcgcagcccttcgacgtggcgaaagtcttcaccggctcggacggcgtgcaggttccgctcgaagacaccatcaagtcgttcaaggcggtggttgcgggcgaatacgaccacctgccggaagcggccttctacatggtcggcggcatcgatgacgtgatcgcgaaagcccagcgcctcgccgctgcggcgtaagggggaaccatggccgataccatgcagttcgatctcgtgtcgccggaacggcggcttgcctccgttgccgcgagcgaggtccgtcttcccggcgtggaaggcgatctgacggcgatgccgggccatgcgcccgtcatcctctcgctgcgtcccggcatcctgaccgtggtcagcgccgcgggcacggccgaatacgccgtgaccggcggcttcgccgaggtttcgggcgagaaggtgaccgttctggccgagcgcggtctgacccgggcggaactgaccgccgcggttcatgccgagatgctggccgaggccaagaaagtcgcggacgccgcgcatccgtcggtggccgatgccgccgcgaagatgctggccgacatggaagcgcttggctcgcacatcaatctctgacgggacatcccgccggatatctcgggccccggtcatcgcgccggggcccttgctttttgcttttgtcttgccgcgccgcatattagcgtgaaggtgcaggcagccggagtgagcgacaggaacggatgaagaagttttcctcgacccggatcggcgtggcccagggatcgctggtgctgttttcggattatctggacggcggcgtgatgtggacgggcgagggcccgcgcgaattgcgcaggctggtggtgttcgacgaagccttccgcgagatcccggcggtgcaggtgtcgctgtcgatgtgggacatcgaccagaagcacaatccgcgcatggacatttccgccgacatggtgacggccgagggcttcgtgatcgtctttcgcacctggggcgacacccgcgtcgcccgcgtccgcgcggactggctggcgatcggcggctgcgccaatgacgacgactgggacgtggcctgatcccgcccggcttgactttccgcccccccgcgccgatggtgcgcgcgactttcccatccaacgaggcccgcccgtgcaacaagatgccccccgctggcagctcgtggtgatcctgtgggggacgaaatatccggtcgccgaactcaacgccctgatcgagaccgtgtggcccgggcctcgag

SEQ ID NO: 35 - DNA, 9810 bp, Unknown
  Description of Unknown Organism:Unknown
gatatcgggcttgtcattttcgattgcgacggggttctggttgattcggaagttctggccgtggccgtcctcatcgcagaactggaccgggcgggcgtgcgggtcgacgaggccttcgtgcatcggcattttctgggccggagcttcccggctgttcaggaggtcgtgcagcgccagttcggcgtgaccctgcccgagaccttccaggtcgaggaacgtgcccggctgctgtcagccttcgagaccggcctgcgggccatgctcggggccgcggagaccgtccgcgcgctgtcggtgccctactgcctcgccacgtcgagcacgccggcccggctcacgcgctcgctggagatcacgggccttgcggccctcttcgagggacgctgcttcaccgcgagccaggtggcgcgcggcaagcccgcgcccgatctgttcctgctcgccgcggccgagatgggcgtcgcgcccgaacgctgcctcgtgatcgaggataccgagcccggcgtgcgcgcaggcctcgcggccgggatgcaggtctggcgcttcaccggcggtagccatttcgcgaaccgatcccccgaggatgcgcccgatgccctgccgcaccggcggttcgacagcttcgaccgtttctacgagaccctgcccggcctgcgccgggccaagtgcgagaccctgacatgatcgaccggcccgagagcgagccgacgcccctcgacgatgccgcgcgcgcgggctggctctattatgtcgcaggcctgactcaggatcagatcgcgcgggagctcggcacctcgcgtcagcgggcgcagcggctggtgagccgggccatctccgaacggctgatccacgtccggctcgagcaccgggtctcgggctgcctgcatctggaagccgcgctcctccggcgcttcgggttgaagctggcccgcgtggcgccgagtctcgggtccgaggtggatcccctgccctccatcgcccccaccgccgccgccgaggtggagcgggtgctgcgctcggagcggccgatggtggtggccttcggcaccggccggtcgctgcgcgccaccgtcgaggagatgacctcgatggtctgcgaacagcacaagatcgtgtcgctcaacggaaatatttctgcggatggctcggcctcctactacgatgtgatcttccgcatcgccgaccgtgtgcgtgcgccgcactatccgatgccgatgccggtcatcgcgcaggatgcggcggagcgggagctgtttcatgcgctaaagcccgtgcagtcggtgctgcggcttgcgcgcaatgccgatgtgaccttcgtcgggctgggacagatgggcgaggacgcgccgctcctgaaggacgggttcatcacgcccgaggagctgaccgagatgcaggatctgggcgccgtcggagaggtggcgggatgggtcttcgactcggagggtcgctacctcgaaaccagcatcaatcagcgggttgcgggcgtccgtgtcgaactttccgaggatcggacggtggtcgccatcgccggtggcaggcgcaagctcgcggcgctgcacgcaggcttaaggggccgtcttttcaacggcctgatcaccgacgagttcacggcgcaggcacttctgtcctgaagccgccgaaaggcgcggcaaaaagtatttgacaggctggcacccctcggtgagtaattattcgccgcacgaaataatgctcaccgtgcaggccagggaggatactgatgaccgcaagatttcgcgccctgatgggcgcgtgcgccgtggctgcgctctcgtccgccgccggcgccgaaaccatcaccgtggcgactgtcaacaacggcgacatgatccgcatgcaggggctcatgtccgagttcaacgcgcagcaccccgacatcaccgtcgagtgggtgacgctcgaggaaaacgtgctgcgccagaaggtcacgaccgacatcgccaccaagggcgggcagttcgacgtgctgaccatcggcacctacgaggttccgatctggggcaagcagggctggctcgtgagcctgaacgacctgccgccggagtatgatgccgacgacatcctgcccgcgatccgcaacggcctgaccgtcgacggcgagctctatgccgcgcccttctacggcgagagctcgatgatcatgtatcgcaaggacctgatggagaaggcggggctgaccatgcccgacgcccccacctgggacttcgtgaaggaagcggcgcagaagatgaccgacaaggatgccgaggtctacggcatctgcctgcgcggcaaggccggctggggcgagaacatggccttcctcagcgccatggccaacagctacggcgcgcgctggttcgacgagaactggcagccgcagttcgacggcgaggcctggaaggccacgctgaccgactatctcgacatgatgacgaactacggcccgcccggcgcctcgaaaaacggcttcaacgagaacctcgcgctgttccagcagggcaagtgcggcatgtggatcgacgcgacggtggccgcctccttcgtgaccaaccccgaggaatccacggtggccgacaaggtgggcttcgcgctcgcccccgataccggcaagggcaagcgggccaactggctcggggcctggaacctcgcgatcccggcgggctcgcagaaggtcgatgccgccaagcagttcatcgcctgggcgacctcgaaggactatgccgagctggtggcctcgaaggaaggctgggccaacgtgcctccggggacgcggacgtcgctctacgagaacccggaatatcagaaggtgccgttcgcgaagatgacgctcgacagcatcaacgcggctgacccgacccacccggcggtcgatccggtgccttacgtcggtgtgcagttcgtggcaatccccgagttccagggcatcggcaccgccgtgggccagcagttctcggcagccctcgcgggctcgatgtcggccgagcaggcgcttcaggcggcccagcagttcacgacgcgcgaaatgacccgcgcgggctacatcaagtgagcccttccgcgggccggccctgagcggccggcccgcaccgcttgccgcttccggccgtatccgccggaggcctttccgccccatcagccccgaggcctccatggcgacccagcattcaaagactgcggcgcgtctgatgatttccccggccgtgatcctcctgttcctgtggatgatcgtgccgctgtcgatgacgctctacttcagcttcctgcgctacaacctcctcatgccggggatggagagcttcaccggctgggacaattactattacttcctgaccgatccggccttctccgcggccctgaccaacacgatcctcctcgtggtcggcgtccttctcatcaccgtggtgggcggggtcctgctcgcgctcctgctcgaccagcccttctgggggcagggcatcgtgcgcgtgctggtgatcgctcccttcttcgtcatgcccaccgtctcggcgctggtctggaagaacatgttcatgaaccccgtgaacgggatgttcgcccatatcgcccgcgggctcggccttccgccgttcgacttcctgtcgcaggcgccgctggcctcgatcatcggcatcgtggcctggcagtggctgcccttcgccacgctgatccttctgacggcgctccagtcgctcgaccgcgagcagatggaggcggccgagatggacggcgcctcggcgctcgaccggttcatccacatcaccgtgccgcacctgacgcgtgccatcaccgtggtggtgctgatccagaccatcttccttctgggcgtcttcgccgagatcctcgtcacgacgaacggtggacccggcaccgcctcgaccaacatcacctacctcgtctatgcgcagtcgctcctgaattacgacgtggggggcgggtcggccggcggcatcgtcgccgtggtgctcgccaatatcgtggcgatcttcctgatgcgcatgatcggcaagaatctggacgcctgacatgtcacgccgcacctcaacccgccgcacgctgatcgtcacgctcgccgcctggacgatagccttcctcatcttcttcccgatcctctggacggtgctgatgagcttcaaatcggaaggagacgccatcaaggcgcccttcgccatgctcttctcggactggaccctgcaatcctacgccgatgtgcaggaacggtcgaactacgcccgccacttcatgaattcggtggtgatctcgctgggctcgaccctcgtggcgctcgccatcgcgatccccgccgcctgggccatggccttcgtgccgggccggcggacgaaggacgtgctgatgtggatgctgtcgaccaagatgatgccggcggtgggcgtgctcatcccgctctatctgatcttccgcgacacgggccttctcgacacgcggatcggcctcgtgatcgtgctcacgctcatcaacctgccgatcgtggtctggatgctctacacctacttcaaggagatcccgggcgagatcctcgaggcggcgcggatggacggggcgacgctcggctccgagatcctctatatcctcacgccgatggccgtgccgggcatcgcctcgacgctgcttctgaacgtgatcctcgcctggaacgaggccttctggacgctgcagctgaccacctcgcgggcggccccgctcacgcagttcatcgcgagctattccagccccgagggcctcttctacgccaaactgtcggcggcctcgaccatggccatcgcgccgatcctgatccttggctggttcagccagaaacaactcgtccgcggcctgaccttcggcgcggtgaagtgaggaccacatgggcaagataaccctgcgcaacgtccagaagcggttcggtgaggcggtcgtcatcccctcgctcgacctcgacatcgaggatggcgagttcgtcgtcttcgtcggcccctcgggctgcggcaaatccacgctcctgcgcctgatcgcgggcctcgaggatgtgtcggacggccagatcatgatcgacgggcgcgacgccaccgagatgccgcccgcgaagcgcggcctcgccatggtgtttcagagctacgcgctctatccgcacatgacggtgaagaagaacatcgccttcccgctgcggatggcgaagatggagccacaggagatcgagcggcgcgtgtcgaacgcggccaagatcctgaacctcaccaactatctcgaccgccgccccggccagctctcgggcgggcaacggcagcgggtggccatcgggcgcgccatcgtgcgcgagccggcggccttcctgttcgacgagccgctctcgaacctcgatgcggcgctgcgggtcaacatgcggctcgagatcaccgagctgcaccagtcgctcgagaccacgatgatctatgtcacccacgatcaggtcgaggccatgaccatggccgacaagatcgtggtgctgaacgcgggccggatcgagcaggtgggctcgcccctcaccctctaccgcaatccggcgaacctcttcgtggcgggcttcatcggcagcccgaagatgaacctgatcgaggggcccgaggccgccaagcacggcgccaccaccatcgggatccgccccgaacatatcgacctgtcgcgcgaggcgggggcgtgggagggcgaggtcggcgtctcggaacatctcggctcggacacgttcctgcatgtgcatgtcgcggggatgcccaccctcaccgtgcggacgggcggagagttcggcgtccatcacggcgaccgggtctggctcacgccgcaggccgacaagatccaccgcttcggcgccgacggaaaggcgctctgacatgcggctcgacggcaagaccgccctcatcaccggctcggcgcgcggcataggccgcgccttcgccgaggcctatgtgcgtgaaggcgcgcgcgtggccatcgccgacatcaacctcgaggcagcccgcgccaccgcggccgagatcggccccgcggcctgcgccatcgccctcgacgtgaccgatcaggccagcatcgaccgctgcgtggccgagcttctcgaccgctggggcagcatcgacatcctcgtgaacaatgcggccctcttcgatctggcgcccatcgtcgagatcacccgcgagagctacgaccggctgttcgcgatcaacgtctcgggcacgctcttcatgatgcaggcggtggcacgggcgatgatcgcgggcggccggggcggcaagatcatcaacatggcaagccaggccggccgccgcggcgaggcgctggtgggcgtctattgcgcgaccaaggccgccgtcatctcgctcacccagagcgcggggctgaacctcatccgccacgggatcaacgtcaatgccatcgccccgggcgtggtggacggcgagcactgggacggggtggatgcgaagttcgccgactacgagaacctgccccgcggcgagaagaagcgtcaggtcggcgcggcggtgcccttcggccgcatgggccgcgccgaggacctgaccggcatggcgatcttcctcgccacgcccgaggccgactacatcgtggcccagacctacaacgtggacggcggcaactggatgagctgaggcccaaggcccggccctccccccgtcgaacgcgccccctatccgaggtaatcccatgacccgctccgtcacccgtccctcctatgaccgcaaggcgctcactcccggcatcgtccatatcggcgtcggcaacttccaccgggcgcatcaggcggtctatctcgacgatctcttcgcgctgggcgagggccacgactgggccatcctcggcgcgggcgtccgcccgaccgatgcgcggatgcgcgaggctctggccgcgcaggacaatctctcgacggtgatcgagctcgatccggcgggccaccgggcccggcaggtgggggcgatggtgggcttcctgccggtcgaggccgacaatgcggccctgatcgaggccatgtcggatccgcgcatccgcatcgtctcgctgaccgtgaccgagggcggctattatgtcgatgcctcgggcgccttcgatccgacgcatcccgatatcgtggccgatgcggcccatcctgcgcggcccgcgaccgccttcggcgcgatcctcgccgccctccgcgcccgccgcgacgcgggggttacacccttcaccgtgatgtcctgcgacaacctccccggcaacggccatgtcacccgcaacgccgtggtgggcctggccgagctctacgacgccgagcttgcgggctgggtgaaggcgcaggtggccttcccgaacggcatggtcgaccgcatcacccccgccaccggcccgcacgagcgcgaactggcgcagggcttcggcctcgccgatccggtgcccgtcacctgcgagccgttccggcagtgggtgatcgaggatcatttccccgccggacgccccgcgctcgagaaggtgggcgtgaccttcaccccgcatgtccatgcctacgaggcgatgaagatccgcatcctgaacgggggccatgcggtgatcgcctatccgtcggcgctcatggacatccagctcgtgcacgcggccatggcccatccgctgatcgcggccttcctgcacaaggtcgaggtcgaggagatcctgccccatgtcccgcccgtgcccgacaccagcatccccgactatcttaccctgatcgagagccgcttctcgaaccccgagatcgccgacacgacgcgcaggctctgcctcgacggttcgaaccggcagccgaagttcatcgtgccgtcgctgcgcgacaatctggcggcgggcacggtgccgaaggggctggtgctgctctcggcgctctggtgccgctactgcttcggcacgacggactcgggcgttgtggtcgagccgaacgatccgaactggacggcgctgcaggaccgggcgcggcgggcgaaggagacgccggccgagtggctggcgatgaccgaagtctacggcgatctggcgcagaacgatcttctggcggccgagttcgcggcagccctcgaggcggtctggcgcgacggggccgaggcggtgctgcggcgcttcctcgcggcctgatccgcagggcccagccgctcggagcaccgaagcggagcccctgccccttgcggcgcaccgtgaggcgaaacgaccgggccaccccggggccaccgcctcggtaacaccatggtatcgcgcaagaatgccggcgcctctgccgaacgggcccggctgccgggcgaggcgccggacttgtcaaggcggcggccctcgggtagagagggcgggcgtggccccgttagcacagtggtagtgcagcgctcttgtaaagcgaaggtcgttcgttcaaatcggacacggggcacgcgatcctccctccgcatcggcgctcgcccccggtctggactgcctcttcggaaggcacctgcccgcttgtgcgccgcgccctttcctcgcttcccaagcgtctgtcacggcttgcggaaagccgtgcgcctcggttctggacagccgccccttgcggtgtaatctgccctcagcgcgcagccggcggacagaagccggcccgccacgtccacaagggaggaatgccatggatcgtcgttcattcatcaccaaggccgccgtgggaggggccgccgcgagcgccctcgccgcgccggcgcttgcccagtccgcgcccaaggtcacctggaggctcgcctcctccttcccgaaatcgctcgacacgatcttcggcggcgccgaagtgctgtcgaagatgctctccgaggccaccgacggcaacttccagatccaggtcttctcggcgggcgagctggtgccgggcctgcaggccgccgacgccgtgaccgagggcaccgtcgaatgctgccacacggtcggctactattactggggcaaggatcccacattcgcgctggccgcggccgtgcccttctcgctgtcggcgcgcggcatcaacgcctggcactaccatggcggcgggatcgacctctacaacgatttcctcgcgcagcacaacatcgtggccttcccgggcggcaacaccggcgtgcagatgggcggctggttccggcgcgagatcaacaccgtggccgacatgcagggcctgaagatgcgggtcggcggctttgcggggaaggtgatggagcgtctgggcgtcgtgccgcagcagatcgcgggcggcgacatctatccggcgctggagaaggggacgatcgacgcgaccgaatgggtcggcccctatgacgacgagaagctcggcttcttcaaggtggcgccctactactactatcccggctggtgggaaggcggcccgaccgtccatttcatgttcaacaagagcgcctacgaggggctgaccccggcctatcagtcgctgctgcgcaccgcctgccacgcggccgatgcgaacatgctccagctctacgactggaagaacccgacggcgatcaagtcgctggtggcgcagggaacccagctcaggcccttcagccccgagatcctgcaggcctgtttcgaggccgcgaacgaggtctatgccgagatggaagcctcgaaccccgccttcaagaagatctgggactcgatcaaggccttccgctccgagcactacacctgggcgcagatcgccgaatacaactacgacaccttcatgatggtgcagcagaacgccggcaagctctgagcccgagcgccgcgcgaaagaggaccccggagccgcgttccggggtcttttcatgggcgacaggggccggcgcg

SEQ ID NO: 36 - DNA, 1886 bp, Unknown
  Description of Unknown Organism:Unknown
tgagtgtctattttttttcgggtttttttaagtgtgaatcacatggttaggagcagttgtcttcaatgtgaccaaccatcccaaggctctaattcaacgtttgggtgtgggggcccgctggcagctgtgtgtgccactgggctgttggtgttggtgctttactccccctcatcgcaaacggctaattggtcggcacagggtatttccacaaaggcgctgtatccggcagtgcctgtgccttccactctgctgcctggaagcgcgcctgccaaacaccagctgcatgtttggagggcacatgcgatgtcggaggccacaacaaacaattcattcaaacagtcattatttgggtacaatgccatctcctccatttggcttcaactggctggtgtggccgccactttctttgcatttggagctttgatggcagctgtaacgcaacgcaaggagatcgccgtcttctccgcctcgggtcaggctgctgagccggagggggcggagcccctgaagcggccttttccgtctcctgctgccaaacctaagccgctcttctccaccccggcaaattccttcagcaacatcttccaggcgcctccatcgctgcgcacggactccacctatggccgaggcccgcgctcgaccagcttcaccgacatcagcaactggccctccaacaacgcactccgcaacccccagtcggtgattgacatcgggggaggagtcgacttcctgggggacagaagccctggaaacccgttcacgcggctgcgggggtccccgagctccaccctcagcaacctcggcatgggcctaggcctggggctgggcaagggcaagggcttcggcaagggcttcggcaaaggccgggggttccccgtggaggaggaggtggaggaggagcaggaggtgctgtcgtgggccgaccgccggcgggcgctggcggaccccgacgccccgccgatgaacgaggacatcaagtacccgcagctgcggctggtgcgggccgtgccgggcggccgggacgagaagctcggtgtgatgtcgaggcaggaggcgctggagctggcggaggcggaagacatcgacctcgtcctcgtcagcatcgacaccgaccccccggtggccaagctagtcaattactcgaagttgaagtacgagtccgagaagaagaagaaggacagccacaagaaggggaaggtgaaggaggtgaaggagctgaaggtgtcccataagatcggccagcacgactacgacgtccgcgtgaagcaggcccgaaagttcctggagggcggccaccgcatcaaggtgtcgatggagttcaaggggcgcgagaaccagttcgtggagatcggccgcgcggtgatgaagcgcttccagaacgacctggcggacatgggcaaggcggacgccgtgcccaagaagctcggcacccggctgatcctgaacctggccccggccggggaggcgctgaaggtgattgcggagcggagggcagagcgcgacaggaaagccgcggctgaggaggagggggagggcgacgacctcgacttcgtggacgagaacgaggacgaggatgtggagggggagggcgaggaggaagaggccgaggagctggaggaggagacagcggaggggacggaggtgccaacccgcagctgatcgccgatccgcgggggacagccacctcccccccggcctccctgccgggggccggcaccatccgtcgttgcggtgcggcgctgccatcaacggccgtccttgagcttaatgctcccgccctccgttggcccgcggcggtcgccaggttgctggcctggctgcccgcagctcctcccctccccgactgacacagtgtggatgaccgtgatgtgcgccttttcgccttc

SEQ ID NO: 37 - DNA, 3015 bp, Unknown
  Description of Unknown Organism:Unknown
ccgctcatctccaggcctccctgagtgcgtacccgagagcggcaagtagagaaaggaacacagatacagcaccatggcctctaggctcgtccgtgtgctggcggccgccatgctggttgcagcggccgtgtcggtcgacgcgcgcttcgtggtgcgcatggtgcaggtggtacaccgccacggtgcgcgcagcgcactcatcgacgacaacacgacggagatttgtggcaccctgtacccgtgcggtgagctgaccggcgagggtgtcgagatggtccgtgctatcggcgagtttgcccgcagccgctacaacaacctctcattggtggagagccctctcttcccgtcgacgcggtacaactcctctgtcgtgcacacacgctccacccacacccagcgcaccatccagagcgcgaccgcctttctgcgcggcctcttccaggacgactacttctacccggtggtgtactcgaccaacagaacgaccgaaacgctgctcagcactgacgcggtgccgtccgtggtgggccgtagctggctcgacaacccggcgctgcacgccgccctcaacccggtgatcgatgagcacctcagctgggacgccatccagagcgctgccaaggacgcatgggtcgagggcctgtgcgcggactacaacgcccgcaccaactgcgtcctcgacatgtacgacgtggccgccgccttcgaggccgccgggcgtcttgacaatgccaccaatctcaaggcggtgtatcccggccttcaggaggtgaacgccgcctggttcaagtatgtcttcagctggaaccacacgagcaagctcgatctcacgcagggctccgcctcgcagaaccttgcgcagacggtgctggccaacatcaacgcccaccgcctctctccgtcgtacaacatgttccagtacagcgctcacgacacaacggtgactcccttggctgtcacgttcggtgaccagggcgagacgacgatgcgtccgcccttcgcggttaccatcttcgtggagctgctccaggacaccgcagatgccagtggctggtacgtgcgcctcatccgcggcaaccctgtgaaggcagccgacggcacctatgtcttccaggagtctggtatcaaggcatactgcatcgatgaagccgggaacaagtacctcgcacacaccggcatctgcccgctgaatagcttccgccgcatggtcgactactcgcgccccgccgtggctgacggtcactgcgccatgacacagactcagtacagcaacatggattgcccgcgcactatcgcggacaacaagccggtgccgtcgcgctgctggctctaccgccacgtttgccctagcaaggcatgcccggacagctacattctctccgcggtcgaccaccagtgctaccccgggcccgacgttacgaaccccaccagcagcagcagcagcgagggtaccaccaccagcagcagcgagggtaccgccaccagcagcagcgacgttaccaccaccagcagcagcgagggtaccgccaccagcagcagcgacgctaccaccagcagcagcgagggtaccgccaccagcagcagcgacgctaccaccagcagcagcagcgacgctaccaccaccagcagcagcgagggtaccaccagcagcagcagcgacgctaccaccagcagcagcgacgctaccaccaccagtagcagcgagggtaccgccaccagcagcagcgacgctaccaccaccagcagcgagggtaccgccaccagcagcagcgacgttaccaccaccagcagcgagggtaccgccaccagcagcagcgacgctaccaccaccagcagcagcgagggtaccaccagcagcagcagcgacgctaccaccagcagcagcgagggtaccgccaccaccagcagcgacgctaccaccagcagcagcagcgagggtaccaccagcagcagcagcgacgctaccaccagcagcagcgacgttaccaccaccagcagcagcagcgagggtaccgccaccagcagcagcgacgctaccaccagcagcagcgagggtaccgccaccaccagcagcgacgctaccaccagcagcagcagcgagggtaccaccagcagcagcagcgacgctaccaccagcagcagcgagggtaccgccaccaccagcagcgacgctaccaccagcagcagcagcgagggtaccaccagcagcagaagtgacgctaccaccagcagcagcgagggtaccgccaccaccagcagcgacgctaccaccagcagcagcagcgagggtaccaccagcagcagcagcgacgctaccaccagcagcagcgagggtaccgccaccaccagcagcgacgctaccaccagcagcagcagcgagggtaccaccagcagcagcagcgacgctaccaccaccagcagcgacgttaccaccaccagcagcagcagcgagggtaccgccaccagcagcagcgacgctaccaccaccagcagcgacgttaccaccaccagcagcagcagcgagggtaccaccaccagcagcagcagcagcagcagcaaaagcacaagttcatcggatgtcccttccttcaaaaagcccgcgaactggagcccgcgcgttctctcgccggaaaggggccgccacattgccggggacatcatccgccgcgtgacgaacggtgttacgatcggtgcgggtgtccgaaagcacgatgagtacagccggcaccgccaacagtagcacaacggcatgtaactcttttgtgcatgtttgaatggagaggaggcttctgtacagcgtacattgtttcgagaaggtatcacaaccgctcgtttcacccccgtcatcttttcattttgatctccgtcgtctcatactgcctttgtgggctctctctgggtgtgggcgcttgtgcgtgtgtcgctgtaaagtcgttgacgccatcgctcttacctgtgggctatttttttaattatggtttattattacttccctctctgcgcgtccctctgcag

SEQ ID NO: 38 - DNA, 38186 bp, Unknown
  Description of Unknown Organism:Unknown
gatccttcctgcctcttccggcgtctggggctccaggcgcccttggcttgcagattgatctccctgatctctgcctccatctgctcacagccttctcccctgtgtgtctctgtctcttcttgtaaattcatccgtcgttggatcagggcccacccggttcctcgtggcctcgccttaactgggccatgtctgcagagaccctatttccacataaggtcctattcacaggaaccgggggtcaggatgtcagcctgtctttctgggagatgtagttcaacccacaacacacatcaaacagttattgagcgccgactgcgtgccctgccgtgtgcttgaaggtcccaccctcaggaagcggggcctagggatggcggccgtgatcacgcaggcagcagagagcagctctgggaagcggggagggacgaggacggggaggcgacatcagcaaggccgtgtgtgagccaggcagggtgtccccggtgtagcacctggctcgggcagaggccccgaggaggggctggaggagctgggcgaggaggcgggcaggacgggcctgacactagggacctcgggccccgggaatgcctctgggggggcgtgtacacccgttgctcccaggaggcacacactgcggttcgcttcgccaagaatgtttaattgcatttgatgactacggtttccattcattcatttgtagagatataacactcagaccacaaaatgcataaaatgcggtggcttttagtattaacagagtgctgcacccgataccacagcctcactccagaacattctcatgggcccaaaaggagacctggggtgttagtcaccagctcactccccgtccccagcccctggcaacccacgctacttagtcattatttaggtgtttaggagttgcaaagtcaaatctttaaacccacatatggccaggcgtggtggctcacgcctgtaatcccagcactttcagaggccgagacgggcagatcacctgaggtcaggagttcgagaccagcctggccaacatggtgaagccccgtctccactaaaaatacaaaattagccgggcgtggtggtgggcgcctgtaatcccagctactctggaggctgagacaggagaatcgcttgaacccaggaggcggcggttgcagtgagccgagattgtgccactgcactccagcctggacaacagagcgagactccgtctcaaaaaaaaaaaaagtaccaaaaagtgccccaggtcataagggcacagctcgatagctggtccctaaagggaacgtggtgtaaccaccacacagaacgaagctggaacgttcctgccgtccttagaagctgcctttgctaaggggaattgccctgacttcccacaccattgattcatctccagacccttggttttcatgttgatttttcaaaaatcacctgatagtctgaccgaatgtagctttccactggtgtgtgtgtgtgtgtgtgtgtgtgtgtgtgtgtgagagagagatggagtctcgctctgtcacccgggctccagtgcagttgtgtgatcttggttcactgtaacctcctcctcccgggttcaagagactcgtgcctcagcctcccgagtagctgggattacaggcacccgccaccacacccagctaattttttgtatttttagtagagatggggtttcaccatgttggccaggctggtctcgaactcctgacatcaggcgatccacccaccttggcctcccagagtgctgggattacaggtgtgagccaccacgcccggccttatttttcccccattttctttttttttttttttgagtcagggtcttgttctgcgctcaggctggagggcagtggtgtggggatcacggctcactgcagcctcgacttcctgcaccaccacgcctggctgttttttttttttccggtagagacgggggtcttaccgtgttgcccaggctggtctagaactcctgggctcaagcgatcctcccgcctcggcctccgcaaatgctgagatcacacgcgtgagcccccgcacccggcctcctttccaccgctcttgtctacagccgcccctcctggtccgattgtattggcagatgtcgccaatacggtgtcaaacggcgaaggggcactgagcgttttttctttctcccgtccttggcggcagcagctcggttccggctacggggctgagcccgtctctcagacgaggaaactggggtccgagaggtgagccggtcccagaggcagggcgagggggaagcgggagtggggtccgcagcggacccagccctgcctcccccctgcaggagatcgtcaacttcaactgccggaagctggtggcctccatgccgctgttcgccaacgccgaccccaacttcgtcacggccatgctgaccaagctcaagttcgaggtcttccagccgggtgactacatcatccgcgaaggcaccatcgggaagaagatgtacttcatccagcacggcgtggtcagcgtgctcactaagggcaacaaggagatgaagctgtccgatggctcctacttcgggggtgagcttgaggggggcgcgcctggagggggagggggcacgcgacccccgcggtgtgcagagccagggggccggggccggggccggggccggggatggggatggggatggggatggggccggggatggggatggggatggggatggggccggggatggggatggggatggggccggggatggggatggggccggggatggggccggggatggggccggggatggggccggggccggcaccagggagagcctgggtgggaagcgcccacgctggccaaggtgcagaggccgggccgtgtgcctgggcggggagggccgcggcgcccgcctcgtccagcaacccccccctgcgcgccacgtgcagagatctgcctgctcacccggggccgccgcacggcgagcgtgcgggctgacacctactgccgcctctattcgctgagcgtggacaacttcaacgaggtgctggaggagtaccccatgatgcggcgcgccttcgagacggtggccatcgaccgcctggaccgcatcggtgagcgggccgggggcgtggccggggcgggtgccctggcgggggaggggcgtggccaaggcatcaggagagtggcttggacagtggcagggggaagggcgtggctgtggcatcaggggcacggttggggcagagacgtggccaaggcatcaggagtgtggccatggcagcaggggcgtggctggggcaggggcagcggctggccgctcctaggacccctttgggtctagaggctgattttctgacctattgtcctacttcagccagaggcagcctgtttcccaagggagggaatgcacagggtgtttgcggttgtgccgaatgctcggtgagcacctgctgtgtgctgggggtgcaggggacagacccgggggcccactcagactcccagggaggcttatggactggtgatgaaatcacacacgactgggctgtgtgccagcagggcaggtggggccggtgggcttccctgagttgggaatgcagagtggagaccagggtaagggatgccatgtggaaacggggaggaagatgtgttcgtggagtggacacagcacatcccaaggccctgaggtggaaaagaggcctagagtccagagagccagggaggcctggaggaggttggggaagaaggggaggccagacacacagggcccagtgggcggcagggagagtttagactaaatcaggagcatcagggagccatggagggttctaggtgggcggaggacctggtcagattgtatccgccaaggcgggccgtgtccaggagggagacggtgacctggcctctcaggggggcagtctctggggcagggagcggcagagccctgatgactggatgtaggcgccagagagatggcggctcatgctgctgttcgtgggaatgggaatgaagaccatggctgaaacgcaggacaggtgcgacggagtggtgtcagggagctccctggtgtacagtaggaagctctccacaacttgctctatacagtgagtatgcaacccgttcctgagtatcaggtgcttaggttataacttctgtatacagcaggtgctcagcacaggctgtgtacaggcaggtgttttcggtatgcctgtggcacactggaggcagtcattacataatcagcgtatacaggtggtacacatgcatacttggtgcacagtgatacctgctccatgtacacagcaggcattaaatacctgtttactgccaggcgcggtggctcacgcctgtagtcccagcactttcggaggccaaggtgggtggatcacgaggtcaggagattgagaccatcctggctaacatggtgaaaccccgtctctactaaaaaaaaaatacaaaaaattagccgggtgtggtggcgggcgcctgtagtcccagctactcgggaggatgaggcaggagaatggtgtgaacccgggaggtggaccttgcagtgggccgagatcgcgccactgcactccagcccgggcgacagagcaagactccgtctcagaaacaaagcaaaacaaaagccctgctttctgtatgcaggtgcttcatgcatgctggctgtgcatagcaggtgctcagcctgtatatggcaggtactcaatatccatactataggccagagatgctacatatgtgcttattgtatacagtaggtggtaaatgcatgcttgctctacacggcaagcactgtgtgcgcacccgcggtgcagagtaggtgctcggtgcccgctgtacgcagcaggcgctccctgtgcacacgctaacgccccctctcccgcaggcaagaagaattccatcctcctgcacaaggtgcagcatgacctcaactcgggcgtattcaacaaccaggagaacgccatcatccaggagatcgtcaagtacgaccgcgagatggtgcagcaggccgagctgggtcagcgcgtgggcctcttcccgccgccgccgccgccgccgcaggtcacctcggccatcgccacgctgcagcaggcggcggccatgagcttctgcccgcaggtggcgcggccgctcgtggggccgctggcgctcggctcgccgcgcctcgtgcgccgcccgcccccggggcccgcacctgccgccgcctcacccgggcccccgccccccgccagccccccgggcgcgcccgccagcccccgggcaccgcggacctcgccctacggcggcctgcccgccgccccccttgctgggcccgccctgcccgcgcgccgcctgagccgcgcgtcgcgcccactgtccgcctcgcagccctcgctgcctcacggcgcccccggccccgcggcctccacacgcccggccagcagctccacaccgcgcttggggcccacgcccgctgcccgggccgccgcgcccagcccggaccgcagggactcggcctcacccggcgccgccggcggcctggacccccaggactccgcgcgctcgcgcctctcgtccaacttgtgaccctcgccgaccgccccgcgggcccaggcgggccgggggcggggccgtcatccagaccaaagccatgccattgcgctgccccggccgccagtccgcccagaagccatagacgagacgtaggtagccgtagttggacggacgggcagggccggcggggcagccccctccgcgcccccggccgtcccccctcatcgccccgcgcccacccccatcgcccctgcccccggcggcggcctcgcgtgcgagggggctcccttcacctcggtgcctcagttcccccagctgtaagacagggacggggcggcccagtggctgagaggagccggctgtggagccccgcccgccccccaccctctaggtggcccccgtccgaggaggatcgttttctaagtgcaatacttggcccgccggcttcccgctgcccccatcgcgctcacgcaataaccggcccggcccccgtccgcgcgcgtcccccggtgacctcggggagcagcaccccgcctccctccagcactggcaccgagaggcaggcctggctgcgcagggcgcgggggggaggctggggtcccgccgccgtgatgaatgtactgacgagccgaggcagcagtgcccccaccgtggccccccacgccccattaacccccacacccccattccgcgcaataaacgacagcattggcgccaagcctggccgcgtgtgattgcccgagacccgcagggcgtgcacccttcctgaagacagtggctcctgggggtggcaaaagagctttatttacacactgacaaggctcacggggtgtcagctgaagaagtaggtggaacgcttcacctgctccaggtcgaaggcccctgcggaggaagcagagcggacggcgtgggtggcgggaaagccccgccctggcccgcagttcgagccacccttgcgaggctgcccacccgcctacctggcttgggcaccgcctgcagtgtctccttcagctggctggcctccaagatcttctggggcctggggttggaagcagggtggggtgaggctgaggccaggttttggggtggggggggaatccaggtagttggggtcagggagcgccttactcagagcagaaccgcttgaccaggaatctggacaggtcctgcaggatgggctcgctgtgcaagcggacaaactgctcccggcacacctgggcaggagtcagaggatccccaggggtgatcaggcaggctctgggcaccacccctacccaacgccccagtgtgggggccccacccatgggtggactgaggctcagactacgggggcacctggttcatgacggagacatcagctgcgtgagtccagtaacagtcgtgcacagagacgaaggtcaggcccttcctgtggcagagcggaggactcctgaagggaggggagctcacagggccacccagtgaccagcatcctggccctgcgctcagccccctccacttgaggtccagggaagcccaccctcctgcaggcctcgccccacccctcgccccgcccctccccaaacatcctgggttaggtatcagtacagggggaggaaatgttcccagaagcctcctcgccccacccctgccgccccccacgctgctgtgggagcctcagctccgagggcggctacgaggtcccctcctgccagggccaccaccccgcatcctgagcattcccagctcccgtggccggtagattctgctggaacgacctccacgtgctccagatctaaccacacatcgcggtgccaagaaatgcccagcaggaaggggcagcgcccatgctcggctctccctgtcgggccacaggaggggagctgccaggaccacctacattcggggcacacagcctcagggcctctacacaggccccacagacacagcagatccactctgcccagtccctgcccccagctagacccagccttgccagctgtgccctgctagccagaagacgcccctgggaggcgagcggcacccacgccgtccggagacgcccacctgtagcagtgcagggcggtgagcatcatgtgggaggagtccagcgagtggatgaagttgggcgggaagccgttcttctgcttacgtgtgttgggctttctgaggacggaacaggtgccggtgggggcggcccagggacacccctaactggccgctgtctccaccgtggctgctctccagacccccggccaggccccagcccgggccccccactcaccggctgatgtctccgttgtgggtgtaggtgatgctctgaattccacctcctatttgctaaaaaggggaaggggccggtgagtcccacccgaggcccagcacggtggtggtacattgaggtggtggtacactggggtagtggcacgctaggatggtggcacactggcgcgggatggggtggcacactggggcggtggtacactggggtggtggtacgctggggcactggtacactgggacgctgttacactgggatggtggcacactggggagggatggggtggtacactgaccttgaccttggagtccaggcgatagggctggatgacggggacgcccaggggtgtgacccactccaccacagagcccatgtgggagatgaggcgggcactctcggtcagccagtgctgtgggacacaggccgtctcagggcagggggctcaggccggggatcccgtccacttgcttagggagtcctggccgagcggggacaggacaggacgtacctggatggcccgggtccccgagaacatctcctgtagactcttgaagacctggcgtacgagatagtgagaggcctcccacacgaactcctgcagagggcgggcagcaggtgcaggtcctcaggggctggcccgttcacgccctactcccccctatttcagagccactgaggcccaaggcctagggcctagcaggggggcaggggaatggggcctggcgcccacgcagtcagcaagaaacgcccaagccctaacaggcagccagtggtctgggggagcagccagggctcctgctgggaggctgggtcgggggcacacccgtctgagttttaaatggcagtgaaaccaacgtgttcgcagcgcgacatgcctggcgcacctggggaaagtcgctcagctcccggaggcgcttctcaatctgcaggcgcccgccatagcgcgtgaccccgtacaccaccgtcatcaccgtctgcttcaccaccttgcgggtgatgaaaccttccagcacctgtgccacccgcatgccccgctgggcgtcctgcctacggaacacctccacctgcacggcgggtgggccgggggcgcgggtcagccccgctagcagcccaggggccaccaagcacccatgaagcccccgccccagccccaccacatcctcaggacaggccaaggtgagggcacctggggccgagactcagggctcacattgcccccacgccgagatgcccccgggcagcagggcacaccctacctgcgcggccacgccgctgtacacgtcctgcggcacatccgagggctccaggttgacggaggcggcgcccacgctgtcgcggcccagagcagcataatgctgcaggccgttgcaagagccgtcctgaggaaggggcggcaaacgggagatggaagctagagaggcagagacgtgtgggaccccaaaccaccccccaggtcgagccgttcctagggccgtgcaccccccagccaagtgcaccggagcccccgcacgctcccgggagagaccaggagccatggctcccgcacactctaggaccacctccagagaataccacgagcgaaggtgaaatctcacaccctcaagtcgagccccaggcccagtgcacactgcacggcctcgggggccagacccagctggctcacctgatggacggggaggtgggagacataggcggcagggtcggaggcgcgcacagcgttcgccacctccatacagcaggccagcgtctgccagggttcctccgcgcccatccaccactttcggccctgcggggacagcggatggggggcagtgaggcccgggcccgatccctgagcccgctgggaggctgtgttgcggggaggtgggaaatggggaggagacgcacacccgtgatagtgaacacgggacgcatgtgggcgagagacggggcggtggctggatgagttctccatagccacggatggaggatgggagctgcgggtggaccgggctgaaacaagcgtgtccggagctgccgggggaggagggtggacagaggacctgggggcgccgggggaggaagcagctcggcggatgcaggggaggggggaacgtggggaacgcgggggccctggggcaggggagaagggagaagcaggacgggcagggggcgcgggggaggagagcgggcgggggacgtgggggcgccaggggagggggaggggaggaggaagacgggcagggggcgccaggggagggggaggggaggaggaagacgggcagggggcgcgggggcgccgggggagggcgcgggggcgccgggggagggcgcgggggtgccgggagggcggggaatgcgggggccccgcccctaccgtcaagggttggtccgcggagtccaggatgtcatccatcacctcctccgcaaaggccaggcgcttccgcagcggctcccgcttcttcaaccccgtgagattgaccaggtggatcttgagccaatccaggccgtgcgggccgagcgggcggccctgggcgaactccagcagggcccgcgccacgtcgctgcccaggtggttgaagtgcggcgggcaggggtaggtgcggccgcggaagtccatgttgtgcggcagccagaagacgcggtcccgcaggtgctgcgccagcgagaggcggtacagcgcctccgcccgcaggctgtgcatctcccgggccaccttctggcagtgcgccagctcacggcgcagctcggccttgcgggcgggcgcggcgctgtgcggcaggtgggcctcgggcggctggggcgcctcggagggcggggccggcacgcctagctgggggcagcccttggcctggaagagctgcagcaccaggtccagcacgcgcccgttgacgcgccaggcgcagttgcccagttgggtgagggcgtccagtgcgccatgcagcgcggtgggcgggcaggtttccagcagctcctggtgctgcgtggcgccttccaccgtgcgcatcagcttggtggggctgagcaggaaagcaccagagtgcggcgatgtccagggcagcggggggcaaagcatgggtacatccaccgcctcgaaggtcagcgtgggctccgcggccttctccagcagctgcacgtaggccgggtgcggcttcaggatgccgatctggggtgcgacaggcagacgggtcagggccccggtgctggggctttcctgttcccaccccttaaacttgggtgagaggggccggctccccggccaacaagaaaccagtgtggcctcccacgaacagaagccacctccagaaacggccggacacctgcatggacacccatggtgtgtcccgagtcctgggaggtactgacggctgcgctgagatcaaggctccgcccaaaggcgccaaccccatggggtccctggtcctcccagcgggatgccccccagctcaggagggcactgcctggcacctgctggacgttgcggaaggaatacacgtggtagagcacggggacaagccgagaggaacgatgcggcttgtccaggctgcatggcatctgcgtagcctgcaccagcatctccgccagcagcttgcccagctccatctgcactggcaggggccagggctgctcccgcagggcctcgggcgcccccagctcctcccagtactgccgcggcaggcagggctcgggcacctgtaggacagggcggtcagggcgctgggcaccggggcccctgagctagatgccccaccgcccgtgcctgacgcccggtggggcatctgtcagcccaagcatacagatgaacagactgaagcttgggtgcaaacccggctgctccagggagggagagcgcccacccaccactggccccagccaggaggagagggggtgcgagcctcacctcggcgtcggaggccagcaagcagaggtacttcctgtagtggttctgcagcgcctgcacctggccactgacccgctgcctctgcaccacgtgccggctgaaagtgcgcgcactcagctcccgggccagggtggtgaaggactcaccttgggcgggcagcgcctgcaggacctgcggaaggcagccgtgagtgcctgcccgccccgcccggggacccggccgcgcggaggaagacgcacctgcaggagcatccgcaccacctcgcgctcgtccagcaggcacaggaaggggtaaagtgagaaccggccctcgtacacctcgcgctctaggcggttcttggtctcccgcagcgcccggcacagtgctttctcccattggtcccgcagggtcttcagggtcttccgctgcgggggatgaacgggcccggtgagccccgtggcagctggtgggacccaggctcacaggacgggggtcaccgcagctccctgcagagacctcatggccctcaaggtccctgctgtgtgttccgggtagctcctcaccccggcctgccctctgccggcttcagcgtgcctgacgcagccaagagcaaaagcccagctgcagtgtgcgcagaagcacaggccaagacccaacctcgggaccccacaagttttccctgagcggcagccaggctgagttcctaggccctgcatgaccagaccagggcatgagcaattcaaccgcatacacggagctcagcccctgcggcggacacgcgaccccggctcagcccctgcggcggacacgggaccccggctcagcccgtgcggtggacacgcgaccccggctcagcccctgcggcggacacgggaccccggctcagcccctaccgcgtgcttgacctccttgcttggcaacgtgggcttctccacggacaccacgcacaccctgctggccagctccatgtggagctgcttctcaaagaggcactgcagggtcttcaagggcaggtgcagcttcgggtaggacacacgcccatcctgcagggatgggggtagtgaggttgggggcttgccagagggcgacctgccctcccaggaccccgagacagcatgggtgcacgcgtttctgcgtctcctgcaagttgctggtggctatcgctgacgcggggaaaggcgggctgcgggtaaagtcagtgccagcagtgcaaaccaaaggccttgaccctcctggcctcgacccctctagaagggacactgggcaccgtgcagggggtggcaggggcggtgatgctgggagctggcagagcctggggagaccgttcactgcacccccagatgttggctgttttctcctcaaactcagaactgtatgaatgtgacccatccagaaatagatgaattaaaaataacaactaaagcctagcgctttgagaatcaaagacgcacgtccacataaaagcttgtacacaaacgttcacagctgcatgactcgcagtcgataagtagaaacagcccaacgtcccataaacggacgaacagacgggcacggcgcggccatccacgcaccggagcatgactcagccctgacccaggtcgcctcccggaggcaccatgaggacgtcacgctcagtgggagatgccaaacacaaaaggtctcgcagtgtgtggtcccatttctatggaatgtccagagcagactcatccacagatggggaggggatggggagtgacggggatggggacgaggcttccttttagggtgatggaacattctagaattagacaaccgtgactacactaaaatcgctgaattacacctttaagagggttttatggcaggtgaattacacctcagtaacagacgagcccactgcgtgcacctggcagccccactcaaacgcactgctctcctgtcaccccaccctctctctgcggcccccgaccacctcgtccccctgagcccacaccctcagggccaagaccctcccagctctgggtcctcccatcttctcagaggaggaagggaggaattcagggcccagcccaggtgagccctgggcaccggggaggcccattggtctgagctgaggctccaggaacccccaaagggcagctataaggactgaagtctgccggggcccacgtgggctcaccttggcatacacgtccctgagcagcttggaggtgttgaccgggggcggcagctgcggcgggaggctgaaggtgggcttcaccttgtgcacggccttcagaacagtggcccgatcctcctcagacagcagaacggcggtgaagagtgcctgcagcttcagcccctcctggctcatctgttccagacacctgtggtgcaggcggcctgctcgagggacgggccagccccacgctgggcttccacagaccccaggggaacctcgtgaccacctcctgctagcctgcaggtctcggtgtggctgtcaggccctctgggggtccccagcccccagcccaggcaccgtcccagatcttaaaaccctgggagggacatggtggggggtgggggccctcccgacaccacctacctttcgatggtcccggcgtcctggtcctgcctccccatgcactggagggcagccgcataggacagcaggtccggagtcaagccggcatccttcaccatgaataacacatataccagctccttgaaggcaccctgggagaccaagccagggtgagggtctggggggatggcccaacctccacatcctccctgctccctggagaccccttctctgtagccaccagctcagcaggggacagggtcaccaggcaggagtggccagctgggcagaccgatgcatccccctgaggttctgacacacaagctccacctgcagaggcagccgcatggcccgccaggtgggactgtgggaggttcacgttcctctgggaggcagcttgttaaacctccagatttgtcaattgtgtggatcttttcaaaggactgacttggcttgactgttctctgctgtttctgccttccatttcatcgatttgttttaatctttgtaacttcctctcatctacttgctttaggtttagtgacagcttcttcttctagtttcctaaggtgaaaggtgacgtatttggtctgagatgtttcactttttttccccccaagatggagtcttgctctgttgcccaggctggagtgcagtggcacaatctcagctgggccgggttctctgcctcccaggttccagcacttctcctgcctcagcctcctgagtagctgggattacaggcacacgccaccacaccagctaattttttgtattcttagcagatacggggtttcaccatgctggccaggctggtctcgaactcctgacatcgtgatccgccagcctcagcctcccaaagtgctgggatgacaggtgtgcaccaccgcgcccggccatcacctttccgaatataggcattttgtgactataaattaccctgcgagcactgtgtcagctgcatcccaggacttctgacaggtggtgttttcattttcattatctccaagtgttttcgaacttcatagtttacttcttctttggaaattttatttaattatttttttagatagagtctcgctctgtcgcccaggctggagtgcagtggcgcaatctcagctcactgtcaacctccgcctcccgggttcaaccgattctcctgcctcagcctcctgagtagctgggactacaggcacatgccaccacacccagctaattattttgtatttttagtagagatggggtttcgccctgttggccaggctggtctccaactcctgacctcaggggatccacccgcctcggcctcccaaagtgctgggattacaggtgtgagccaccacgcccagccatgtatagcttaaatatcccctgcaatttttttttttttcatttaatttttggccaggcacagtggctcatgcctgtaaccccagcactttgggaggccaagacaggaggatcacaaggtcaggagtttaagaccagcctggccaacatagtgaaaccccatctccactaaaaatacaaaaaaaaaaaaaaaaaattagctgggcgtggtggctcatgcctgtgctccctccactaaaaatacaaaaaaaaaaaaaaattagctgggcgtggtggcacatgcctgtaatctcagctactgggagcctggggcaggagaatcacttgaacgcagaaagcggaaattgcggtaagccgggatctcaccactgcactccagcctgggagacagaaactttgctgtcgacagacttggagactctgtcttaaaatatacacacacacacatatatatatatatataaaataacatatatatataatttttttcttgtattcatttttcctgacatccctgttctgagcaatttctcctttgacccagtggctgcttaagagtggcctgtaactgtaacagactattccaaagggaaaaaaattcccttacatcctcccaccccatagtcctgcagctgaagacatgctgtgacatgaggtggccacacaccagagaccagagacatgagttttggggcatttttttttttttttttttttgagacggagtctcgctctgtcgcccaggctggagtgcagtggctcgatctcggctcactgcaagctctgcctcccaggttcactccatcctcctgcctcagcctcccaagtagctgggactgcaggcgcccgccaccacacccggctaattttttgtatatttttagtagagacggggtttcactgtgttagccaggatggtctcatctcctgacctcgtgatccgcccgcctcagcctcccaaagtgctgggattacaggcgtgagccactgtgcccggccggttttggggcagtttctaaacaacctctgtatggtagacctcactggccacacatagtccttaaattgaaatattcagttcttccctttcaccagcttcaagtgttcagtagcacacacagctgttggcagatgcggaaaattcccaacatcatagaaagttctactggatggtgctggttagaatacgtggccgggcgcggtcgctcacgcctgtaatcccagcacttagggaggctgaggcgggcggattacctgaggtcaggagtttgagaccagcccggccaacatggcaaaagcccgtctctactaaaaatacaaaaattggccgggcgtggtggtgagtccctgtaatcccagccactcaggaggctgcggcagggagaattattgaacccaggaggcggaggctgtagtgagccgagatcatggcactgcaccctagcctgggcaacagacagagagtctatctcaaaaaaaaaaaaaaaaaaaagatagaagcaatgccttagcctggctaacatgctgaaaccccacctctactaaaaataaaaattaaaacaattatccgggggtggtggcacacgcctgtaatcccagctgctcgggaggctgagctcgcagtccagcgacatccaggactgctggccaccccggaacgctgggagaggcaggaggggcccctgctagagcctctggagagacttcgggtctgcagacatcttgattccagacttctgggctcgtgctaagagtgcgtttctgctgtgcaagccgccaggtttgggacactttcgtaggggccgatcccaaaagcgccctgttacagtgtgggctctctgcccagggaatccagggggcttgtgaccttggaggggaaaatacacgaccctcatcctcagtcctcccggagtctggcgccccctgcagcaaggaggaaccaggcagcacgccgcctccacctcgcggtaagagcactgcggacttcaccgcaagactggccccacctgatcctgaatttcgctgtttgatgcgttaataaagaagcacatcaagttctctaccacgaattggtcttaatattgcgatatctgtattttaatataatagtatcccatgtttacccaaatattaagagaagcttttactgttgtttctcaaattagggctgaaggatcatggggggggagaaagctgggaacgtttgctgctttgaaagggtgtgtaaacaacaccctccaaaacaaccaagagttccgaggagaaactttggccggatacggtggctcacgcctgtaatctcagctcctcgggaggctcaggggggcagatcacgaggtcaggagtttgagaccagcttggccaacacggtgaaacccccgtctctactcaaaatacaaaaattaatcgggggtggtggcgggcacctgtaactccagctacttaggaggctgaggcaggataatcacttgaacctgggaggtggaggtggccatgagccgagatcgcaccaccgcactccaacctagtaacagggagagtatgtcccagaaaacaaataaataaacaaacaaaaagaaaacggcaagggaaattggaaaatactccagatgaaccacaacgaagatgggtgggatacatctaaagctgtgctcagagggaatgcggcgccagtgaacacccacatttcacacagaaggatctcagcacagcagcccgaccttccacctcaggaaaccagaaaaaggagcaaagtcaaccccaacaccaaagcctcatcctgacgagggctctgcaggctgccccccgacgaggccaaaagcacccctgcccagacagattcacgagccccgagaaagaacggaaggaaatgctcaaggcattagcagaatttctccctacttttttggtcattttcaaaatttgagagtcacacgtgatttgtatttgaaaagcctaaaagaattattaaaataaaaaacaaaggacttgaacctgggggctaagagagaaaagtccagtctaaatgagggcaagttcctgtctccaacgaccagggcaggtggcccggctcccggctgcactcacctgccgcgcccagccaagcatcacggcgttgtacatgtccagcgtgagcagcttccgcttctgccgctggccgtggtggacgaccagcaggtggtgggcgaggggcagctggtcagtgagcaggcagcacttgaagaaggccaggagcctctgctgctgacctgagagctgggcctgcgagtgctgccccgacggggcctgctccacatcgaggctcagcttcccaggggcctcctgcagcagccgggccagctgctcctcccaggggctctcgggggcctggcgcgtgcagtcctccaggcacccggccatctgcttgctcaggagccggggctccacctgcaggcgcctggtcagcgccttgaactccccgctctggaatggcatctgcagcttcgccttcaaccgctgcatacgcatctgctgggtccgcttatccttctccagtatctttgcccagcggccacagggcaccggggtggcatccttggcccccatctggaccttcctgggtggctggaggctaccatctccactgccacattctgggagccgcgccacatccaccctgttcaccaccacctccgacacgctctcagcctgcagctgccgcacccgcgcctggagcactgtgaggggcagaaggcgaggacatgagagggaccccctccccattcgagcacccgtctctctggaccctgagccaggccaggaggtgcaggtggctgagctcgctgggacccaaggcgtgaattcctcatacttgccaacaacgttgtaaggtctgcccgctgctttccagacacacgcaccccaccacctccgcacctccccacccgagcctcacagaactcagcagccctaacaagctgccaccgaaacctgcagcaccacgtctccccggtcactggccgctcagaccctccaggtgcacaggcccagaacccggggtctgtgacaactccctccgtccacctctcagtacctcctctgggcttgcctccagaatctatccaggtggcccccgcctcccctgcccctctcactgtctagctcagggcctctgcacagactcccaggaccctgaaccgcccactccctggctcaaccatggcctgcaagttcgcaccccgcctcagcaagacccccccagctggtggagctgccacacacacactcctaggctcccagtgtctacaccggtggacgctgagccactagctcgcagggaaaacgcggctcctgctcgtgccgcctcaggttgcatttttgccaaccaatcaatgcctaagtgttctgtatctctttaaagaagccttgttggaaatctattgctggccgggcatggcggctcacgtcggtcatcccagcactttgggaggccgaggcaggaagatcacctaaggtcaggagttcgagaccagcctggccaacatggtgaaaccccgtctctattagaaatccaaaaaattagctgggcgtggtggcatgtgtctatagtaccagctacttgggaggctgaggcaggagaattgcttgagcctgggaggcagaggttgcagtgactcaagatagcgccattgaactccagcctgggcaacagaacaataatccatctaaaaaaaaaagactgttgaaataagccgggtacagggccgcgcacctgtggtcccagctactccggtggctgaggtgaaagaatcacctaagcctaggagttcctggctgctgtgagccgtgatcaggccaccgtgctgcagcctgagagacagagcaggaccctgtctcaaaaaaaaaaagggggggggggacccaggtgtccagatgtggtggctcacgcctgtaatcccagcactttaggaggccgaggcaggcggatcacgaggtcaggagatcaagaccatcctggctaacacggtgaaaccccgtccctactaaaaatacgaaaaattaaccgggcgtggtggtgcgcgcctgtagttccagctactcgggaggttgaggcaggagaattgcttgaactcgggaggcggaggctgcagtgagccaagatcgcaccattgcactccagcctagcaacagattgagaatccgtctcaagaaaaaaaaaattgctgaaataaaaagacaagcgtgatgtccgccttcagagtgctccaaaactcaggagatacttttaggattaacagttgagagctttgttttgttttgttttgtttttgagatggaatttccctcgttgcccaggctagagtgcaatggcatgatctcggctcaccgcaacctccaccttccgggttcaagcgattctcctgtctcagtctccccgggttcaagcgattttcctgcctcagcctcctgagtagctggcactgcaggcgttcaccaccatgcccagctaatttttgtatttttagtagagacagtgtttcaccatgttggccaggctggtcttgaactcatgacctcttgatccgcccgcctcggcctcccaaagtgctgggattacaggcgtgagccaccgcaccaggcctcggacccttgacctcttgatccgcccaccttggccacccaaaagtgctgggagtacaggcgtgagccaccgcaccaggcctcgaacccccgacctcttgatccgcccacctcggccacccaaaagtgctgggattacaggcgtgagccaccgcacctggccaggttttttccctttataaaggttctcccgcctctcccttcccggctgcctaatggacgcagacaggatgtgggacagaagcaccggcgggaagcaagcacagggaagctcccacctccctcccacaccaccagccaggccaggacgagggcctgccaccgctggagcctgggctgtccctcccaagtttcgcagtcatccagtctccattaggcgcctaccccccagagccaagccaggacagctgagtcagttcagggttcacatcctggctctgcacatgtggccttggcggcggggccggggggggggtctctccagacataatcttgggcctcacctatgtccctggaaagtgggagcacctggtggggttctggggagggggaattacgagagctccaggaaggagcctgctcagcaaggacagggcccatgagcggtgcaagagatgtttcagcaacgccgtctgggcgtgtcctgggacccgagaggtggagaccgccctcagcctgtctcagaatctgagcctttgccttttctcccggcagcagggagcggactctcctctcccgggccgccgtgggggtcgcgctcaccctccagcagctccacgtggccccagtccttcctgcggtcttggtcttgctcctgggggctggcggacgagctcctcctggggccgcagacgccaccggcggtccctgcgggaaagacgagagcggctgagcggggccgggcgtgtgggcgggggcctccataaaggcagaagccgaagggtcgaagggcaaaggagccctaaacgcagcggaaactctcggagcacgggcttaagttggaaagaaactaagacagcgaaggtggaagggccccgccgcggcgaacacgggcgcggaaccgccgagagagggttcctcgcactcgaggtgcagcaggtcaaaggttaagagccctaaacaccacacctggggtcaggaggctgcataagaaaccacgagtcaaaggtcagactgcacggaggagcctcagtcgaaaagcgggcaagggcgagtggaaagcggggccgggtcggtgggctgcgcacgcccaggtgcaaagaggcaaaggtcaaagcgccaaaggccccggccgcgcggggaggagcccacgccgtggcccccgggctgcctggccgtctccctttgtgttaccttctttgccggggagtcccgggcggccgcaaggccgtagggctcgtttgagccccgccgctccgcggccccagcaaagtgccgacattacgcacgccgctccaggccaccccaccggcccgcgcctgcgcatgcgcccgcgccgcctgccgggagttgtggtttcatggtcgacggaggctgcgaagggaaaccccagccggaagtagactcccaggatgcagcggaggcgcgaaggcatgcgccggtggacgctctgattggttcctcctgctgtttttaaagggagggggcgggacagagctgttgccgtggcaactgggaggcactctcaggctgttttcccgaggacctcaaatccggactttttttctgtttttctttcttttttggttttgttttggacgcgttgtggcccaggctggagtgcagtggcgtgatcatagctcagtgcagcttcgaactgctggggtaaagagatcctcgcccctcggcttcccaaagcgctgggattgcagacgccgccaccgtgcccggctttttttttttttttttcaaggcatactcatctaataacgaggacagcatctgcaatttagagattcctgtccgcaaccttcattgctccaacgacaacttttgggtaagagtcattaggatgccgtctatcatggaggaagctgaggctcagagagggccaccaagttgctggaagacacagcacgtgcgacctcagggaggctgcaaggagagaaagccccagtccgcgagactcccagcctccagcttcagtttaccctccaatccccaagccctcaggggcaggagccgaatggagcggcaggcttggattcacctgctaagtggggtgaggtcaagggaatgaaataaacctcggagcctagagcctgccctggtctccgcgtgatcctgcctaggaggagcagggcgggagctttagaatggaacctggaaggtgtgcccacctgtgtcgttcagccggggcagcaggccagaggcgggagcgcctgctgtggggcagtaggcttgggaagggtgagaataggaatatctgggggtaactgtgttccaggctaatatcccagttgcaaaggggagctggtttggtggctcaggcctgtcatcccagcactttgggaggctgaggcgggcggatcacctaaggtcagagttcgagaccagcttggcaaatacgcaagcatgcctggcaacatggcaaaaccccgtctctagtaaaaatacaaaaattatccgggggtggtggcgggcacctgtaatcccagctactcgggaggctgaggcaggagaatcgcttgaacccgggaggcggaggttgcagtgagccaagatctcgccactgcactccagcctgggtgacagagcgagaacctgtctcaaaaaaaaaaaagtgcaaagggaggtcagttcagtgcctcaggcctgtaatcccagcactttgggaggctgcggcgggaggatcgcttgagcccaggagttccagacaagccttgggcaaccgagatactgagacccagtctccaccaaaggaaaaaaagaaattagccaggcatggtggtgcacacctgtggtcccagatactcgggaggctgaggcaggaggactgcttgagcccaggaggtttagactgcagtgagctgagatggcgccactgtactccagcctgggttgacagaacaggaccctgtctcaaaacaaaacaagtgcaaaggccctgaggcaggaacaagcgtggacagaggagcaatttgagcagagtggggctggggagagggagcaaagatgtagctggggctcagttagggggcctgaccacacgggggctcgggggcctcagctcaagctatcctccatccccaaaccctggcacttcagtttccccatcagcccagaacgaggactcgacctcactctggaagggcctggcagcctccttacagcacattccagacgctgctgccgacgcctgcgtgagcgcactgatgccaccggctgggaatgttttcgacagacggcagcaccctccctcacctgcctcagtccacctcagggtgccccagcgggctgtgacctcagacctcacccactactggggtcacctgcctggccctgaatcagccaggcctggtgtgccaagacctacagacaccccctgcacccctgcaggctggcagagccagaaacttgggtggaaaccgacttctgaactatttcaccattccttatgcgttagtcttttcttttatttgatgagatcccagcactttgggaggccgaggcgggcggatcacgtgaggtcaggagtttgagaccagcctggccaacatggtgaaaccccgtctctactaaaaatacgaaaattagccgggcatggtggcctgtgcctgtaatcccagctactcaggaggccaagggaggaaaatcacttgaacctgagaggtggaggttacagtgagccaagatcgcaccactgcactccagccttgggcaatgtagccaaaccccatcactacaaataatacaaaaaaattttgttggctgtgatggtgcctgcctgtggccccatctacttgggaggctgaggtgggaagatgtagaattgcttgagccaggaggcagaggctgcagtgagctgtgattgagccactgcactccagcctgggcgacagagcgagaccctgtctcaaaaaaaaaagaacataatctgggttttggaataagacagcagtttctgaaacagctcattgcccaaattccagcctcgcaactctgtagccgccaccaccccccagccccaccatttattttaactacatctgtctccaccactcctgtattaagtaaatgcaatattggctggtcatggtggctcatgcctgtaattccagcactttgggaggctgaggcaggcagatcccctgaggtcaggagttcgagactggcctggccaacgtggtgaaaccctgtctccactaaaaattcaaaaattagccggacgtggtagtgggtggtgcctgtaatcccagctacttgggaggctgaggtaagagaaatgcttgaatccaagagactgaggttgcagtgagctgagatctcgccgctgcactccagcctgaacgacagagcgagactccgtctcaaaaataaattaataaatacaacattaattatttttcttgcttaagttttacgaagagacttaatatcaccatcaaaagtgggaaaccatatatctggccgggcgtggtggctcccgcctgtcatcccagcactacgggaggccgaggcgggcggatcccctgaggccgggagctggagaccagcctggctaacatggtgaaaccctcatctccaataaaaataacaaaaattagccgggcatggtgggtgcctgtaatcccagctattcaggaggctgaggcagaagaatcacttgaacccgggaggcggaggttgcagggagccgagatcacaccactgccctccggcctgggcgacagagcgagactctgtctaaaaacaaaacaaaacaaaacccaaccaagcaaaccccacagagtcgagaatcgctagatggaaggggatggcccaggtccctggagcccctgtgacaaattaccacaaactcggtgccttaaagcaacgttcattttcttacatttctggaaatgaaaagtccaaaatcaggactgcggggctgaagtcaaggtgtgtggaggcctcgctccctccagaggccctggggctccttcctgcctctcccagcttttgaaggctccaggtgtgcttggcctgcggccacatcactcccgtctcggtctctgtggtcgcactgcagcctcctcgtctgcctgtgtgaaatctcctcctgtctccgtattgtgaccgcgtttaggatgccccaggacaatcttctccatatcgttcagatcttcatggtgtcaatatattgagactctttttccaaataaggcaaatgtcacattctagggatcagggtggggacttacctttgggccaaccacagaggctacaaagaggaagacaccactcaatacaaagcgtgcgccagcccagccctgatcggtgtttgttgttgttgtttttgtttgagacagagtctcgctctgtcgcccaggctggagggcagtggcatgatctcagctcattgcaacctccgcctcctgggttgtatagattctcctgcctcagcctcctgagtagctgggattacaggcgtgaaaaggagcaaggctctgccccagccacagcgcggatgcaccttgaggatgtcatgctcagtgaaagacgccagacacagaaggacacacagtgtgtgatcccctttatatgaaatgtccacaacaggcccatccacagaggcaggaaggggatgtgtgggtgccgggggctggcagaggggatgagtgacagctgatggggcttcttcttgcggtgatggaatcttctggaactagacagtcgtggtggttgcacaactctacgaggtactaaaatcactgaactggctgggtgcagtggctcatgcctgtaatcccagcactttgggaggcagaagcaggtagatcacgaggtcaggagtttgagaccagcctggccaacatggtaaaactctgtctctactaaaaatacaaaaattagctgggtgtggtggcaggtgcctgtaatcccagctactcaggaggctgaggcaggagaatcgcttgaaccagggaggcagagtttgcagtgagccgagatcgcaccactgcactccagtctgggtgacagagccagactccgtctcaaagaaataataataaaataaaatcactgaactgtacagtgtaagtgggtgaattgtgtggtatatgagtgatgtttccgaggtgtcattaaagaaactcagacgcctggggtggggccagtctcaccgctgtgggtcccatccccatcatttctcacaaggccctcagatcacccttccgcggtggggggcggacactctaagaagggaagacctgggctcctgctggcgagaaggcggtggacatttcttcagtgtctggtgccgcgccctctgcccagcgtgctccgtggagggtctcattgtcttcctccagacgtctctttactggcccattttacagaggcggaaccgaagcttggggtgttggccacagggctctagtgtgggaagccaggccaggctggacctcagccatggggacccctgtccctgagactgtggcacctgccacaccctctgtgtgacccgcctaagccaggaagagagggtcaggagatgcctgagccaccaagaaggcatcccagcgtccagccagaccggttatccctccagagggctccccggcaggacaggctggtcgccatgtcttcagcctggtgctatttaaaggtgggtgccacctggggctgtggccgcagggccaggactgggctgctgggagctgtgtccccacagcggaggtcgccgcccctctcaggcctcggtttccccagttgtcaatgcctccacttggctgtgagtctgtgagggtcactgtgctcaccttttggggcccagcgcatggggcaggcagaggaagggtgggggccagccgccttgctgggtggttccccgtggggcctggggtatggctctaagggaggagcaagtgtgggtgcgaatggggccgccccattcctgccgcctccgacgtgccccgccagccggccaccgacaggtctacgtggctatcctccctcctgcccacctacctgcccaaacacacgtccccagtcgtcacctgcccacccacccgcgcattcccacacccttgtgggcctggctttcgggaaactacaatttgcggggagagaagtcccacgagggcatgccccggagcctggctggtcccacggctgacgcacgcggcaggacctcccgtgtccatctctgtccccaagcatctccgcctctgcccctctctgtctctgtgtctctctcgtctctcccggtcatcttccttgtgtctcttgactgccgccgtctttctgtctctgtctccctccgggtctctgtctccctccaggtctctgcggcccgcgtctcacactcccgcccccgcaacccgaggtcctagcccgcccggggactcggctgactcacggacacgccccgcgagacaaacaacaaacgcgcggaggccgagcgcggagtcccgcacggccgcgcccctgtgcacctggcccccgcccccgagacgtcccattggccggcgccctagcctggtcccgcccaagtggaccccgcccccgccccgaggcaccccattggccggcgtccccgccccagcgaacccggccccgcccccgaggcgccccattggccccgccgcgcgaaggcagagccgcggacgcccgggagcgacgagcgcgcagcgaaccgggtgccgggtcatgcgccgccgcctgtggctgggcctggcctggctgctgctggcgcgggcgccggacgccgcgggaaccccgagcgcgtcgcggggaccgcgcagctacccgcacctggagggcgacgtgcgctggcggcgcctcttctcctccactcacttcttcctgcgcgtggatcccggcggccgcgtgcagggcacccgctggcgccacggccaggacagtgagtgcggggcggcgggggcctggggtggggaggcggcgggtgacggcaacgcggccgccgtcttcacggtgacctgcgcccgcgggggagtcccggaggctcctctgtgcagcctcggcctcagtttccgtggtctgtgagatgggtgcagcctgcctggtgggagggttgcactgttaaagcgaaggctgcagcggcggacccggctcaggggcagagaagcgtccgtgtggtacaaccctgtgggtggggccacccatctgcaggtgggaaactgaggctccagaggggctggggcaggcccagctgcatggcggaagcggcggggggctgacctccggactcctgacatcacagaatccagtcagggctgcctgagtcggggccccctctgcttcttcccagacaccccatctggcaggtgaggacaaggaggcacacagaagggatgggacctgcccagggtcacactgacaggggtggcggagctgggtccccacagggcccaggacgtcacggagcgggcgtctctgtccccagggtctgccgagcacactgaggtaggccctcagtgtttgtggaatgtcaggagcaagaggagaggctgggcacagcaggggatgtgggtacctggaggccaggggagtcggtgtccccgccgggcggggggcactgggaagggggcccgggcccgctggctgccgcctgaatcaccaccatcagggcaggtaatcaccccctgtccttcccaccgctttcatctgggcgccaaggccctcattaggccgcacgtgacgagggcggacaggggactggctgggccggtccatccatggcgggcatggccaggcggggtggcctcgggccggggcagaggcctggctccgctgcctgacctggaacagtctctgcctctctccaagcctcggtttccccagctggacggtgatgggggtgagggctagctgagggctctcctgcccttcgtgcattcgctggtcactaatcgggcaccttgtgggtgctgtgctccgcatgggggacccagtggtgacagagacgcccaccctcctggggctcccagagcagaggcgcgcagcagttagacacgtgaacaagggcgcaggtgggtgcacagaacagtgaacggttggccgggtgcagtggctcacgtcggtaatcccagcactttgggaggccgaggcgggcagatcacgaggtcaggagatcgagaccatcccggctaacacggtgaaaccccgtctctaccaaaaatacaaaaattagccgggtgtggtggcgggcgcctgtagtcccagctactcgggaggctgaggcaggagaatgacgtgaagccgggaggtggagcttgcagtgagctgagatcgcgccactgccctccaccctgggcgacagagcgagactccgtctcaaaaaaaaaaaaaaaaaaagaacagtgaatgacgtgaacaagggtgcaggtgggtgcgcagaacagtgaacggcggtgttgggaggcaccttgccaggggaggggaggtgcagggcgaggaaggggccaggggagatcgtgacacagacgccccagaacaaccacctcaaagacgttcctgtgtgtcctggaaggtcgggctgggaggctgccccgaggagctttcactttgacagggagctggccgggcacgcagggaactgtacacccagctgacaaagcggcagacacccaggccggggtgagcgagtgtgggtgaggagtggcggctggccccagggtccttgctggacaagacacttcagctcagggtggggcagggctcacccagggctacccacagacgatggcgtccaaatctggctctgccactcccaggcctcaactggcccctctgcaacgtgggctgctgagcgggcttggtaggacagctggcatacagtcggcgctcaagcatgtctgtggtgtcccataaaccaccggtgtcccactctaggccactgccagcccggcctccagtccagagtcccagtccggagtcccagtgactgtgcgtgggccgggcagctgagctgtgagggccgggctgggggctccatatggggtggtgtgagctgtgagggccgggctgggggctccatatggggtggtgtgagctgtgagggccgggctgggggctccatatggggtggtgtgagctgtgagggccgggctgggggctccatatggggtggtgtgagctgtgagggccgggctggggggtccctggggtggtgtgagctgtgagggccgggctggggggtctctggggtggtgtgagctgtgagggccgggctgggggctccatatggggtggtgtgagctgtgagggccgggctgggggctccatatggggtggtgtgagctgtgagggccgggctggggggtccctggggtggtgtgagctgtgagggccgggctgggggctccctggggtggtgtgagctctgagggccgggctggggggtctctggggtggtgtgagctgtgagggccgggctgggggctccatatggggtggtgtgagctctgagggccgggctgggggctccctggggtgctgctggtcgctggctcattgacagttatcagtggtctgggtgggccctgccccttctgactcccacatcccaggaaccctttcccaaccttcctcgtggtgttgctgcccccctgacgtccgtccctctgggtgtgtgggagcccccccgccatacacacacacagatgctgctcttgggctgagctgcagggacagcgctgacctggccctcccacggggtcctcatcgatctctgcactcccccagctcgtgggggccgtcctgcttcccgttccctctgcctgctccttgctcctccctcacatgctggggggggctcctggtgtcagtcacggctctgggggatcctgagtgtccgtcgtggtcgggaggggactcgtggtcccgggggtctcctggtatctgtcgtggtcctgagggccctgcacgaagcacagcggacagcagcggtgctgggggtgagccagcaaggccctccccgacccccgcctcccccaggcatcctggagatccgctctgtacacgtgggcgtcgtggtcatcaaagcagtgtcctcaggcttctacgtggccatgaaccgccggggccgcctctacgggtcggtgagtgccgggcagggctgggcggcgcgggcagggtggggagggtgggccggcctcacccccgcccgcagcgactctacaccgtggactgcaggttccgggagcgcatcgaagagaacggccacaacacctacgcctcacagcgctggcgccgccgcggccagcccatgttcctggcgctggacaggaggggggggccccggccaggcggccggacgcggcggtaccacctgtccgcccacttcctgcccgtcctggtctcctgaggccctgagaggccggcggctccccaaggtgcctgggctggtggcgaggggcccggccacgcttgttcttccccctgcgggctctgtaagcgctgagtgcccaccgtgtgcgggcgctgtggacacagcccaggagccctccaggggggtcccagcctgagggggtggtggccaccaagcaggttcaatcctgagttggggacctcgaggacccaacagggcgcctctcgggctgaaggacgcagacgtcgaaaggtcgagggggacgtcccaggcagggcccggcagaggcaggggctcggggtggggagcacgttgggagtgggggcaggagcggaggggaggggagggggccggggagacggtgacagacgccgcagaacaccagcctcgaagccggtcccgtcccgggaatctgcaaatacaacgccttgcgaggacaaaggcacctgcaggtgggacggagatggaggagcatccagggtggggggtccagggccccagtgtcctcacagggtcctcacgacaggaggcgggacagtgagagccagagagagatggggatgggccgcgctgtggccgtgaaggggaggaagggccctaagctgagggacgtgggtgcctccagatgctggggaaggcgggaacggttccgcactggagcccccgggagggaccggcctgctcctgccttgatatgagcccagtgggacccagtttggactctggcctccagaaccgccagaaaataaacgtagtaagccatcaactttgtggtcttttgttacagcagacgtcggaaatatgcacacggtgtctgaaactgttctcatgacaaaataagcctcagatcccccggggaagggcggaggccaacgcctcggtgttcctccgatcccccgggaagggcggaggccgacgcctcggtgttcctcggatcccccgggaagggcagaggccgacgcctcggtgctcctcagatcccccgggaagggcagaggctgagggcaggagccgtgctgggtgcagggcaggcctgggggcttcatgccgctgtcctgcgggacgcagagagggctggccgtcggtgtgggggcgcccccacctgtgcccagcgccctcctgacatcctgactccgctgggacttctgcctacagccctgggagtcaaactccagcctctcagagaaaaggtcagagccaagagccccacagcctggagccaggcagtgacaccctgggcctgtctccccttctgtgtgtggggcgacagcagcatcgccctggtgaagtccccggggacggccagggctccatccccagccgccgccttccacataaatacaggaagactgggccgaggcacttgctgggaggtgctgagcagcctgacacggaaaacccttctgggaagggagggtcgtgcccggcccgagagcttctgctcaccctgcagacagaagcgagccccaccccaggggacaccaggcggcctctggggacatctttggctggcatggagtgggtggaggacagggctgcacccaggatgtccccaggttggcagtgtgaggggagatcggcccacgttggccagtcggagggcgtcgccacttgagttgtcactgggagctgcacaggtcaccacagctgaaataaaacttgctggcaccccacgcaggaacgtaacatgtgcctcgaagaaacgggtcagcaggccgggcgcgggggctcacgcctgtcatcccagcactttgggaggccgaggcgggtggatcacgaggtcaggagatcaaggccatcttggtcaacatggtgaaaccccgtgtctactaaaaatacaaaaaattagccgggcgtggtggcgggcgcctgtaattccagctacttgagaggctgaggcggggaatcgcttgaatccgggaggcggaggttgcagtgagctgagatcgcgccactgcactccagcctgggcgacagagcgagactccgtctcaaaaaaaaaaaaaaaaaagaaacaggtcagcagttgtttctttgtttctaaaacagagcgtggaatgggcgtacagctccgcacatcccagggcagtgaaatcccggttcacacagagccctcagcagcttattcgcaagcccaaacctggggacccccgttgtcctcaggcagtgaggtgggggccccccaacagagaggagcggcctgggggcacagaaccagcggctccccaggaaatcgccagcagtgaaaataagacaaccccaaactgttgcaaactgtgcttccgcttacgaagcactcctgagcggcagggcggatggggagagggcggctgcaggcgcgaggggcccggggacgcaggggtgcgggccttaccagggccctgtcctgtcgtgcagcaggctcctggggcagggaagacaccaggggcggccacttcttactgctgtctgacctcgagcaatgcggcctcacagcccccaccagggtgccggtgtcctctgggcccagcgcccccgaggctcatgcctgggtggggcgaaccaatcggtcctgctcctctggccactccacgcgagggaagtcccagcctcacaggcaggcgcacaccccggcagcatctctgacaaaggccctccagttccgagtctccaggtcccgccgctgcaagcctcacctgcccagccctcctctccagctccaactccaactcccaagaaccaccacggacacacagaacccgagccttgtctccctcaacgcctcctgactcaaaactccatcttccaacaggaaaacggctcggccgggggactgtgacccggagcaggcggcccagcctgtcgcgcagactcggggcctaaaacacttgttctctcagtccggagatcaaggacgatccgaggtaacctccctacctcggtgtcctccatgcaacctcgtcttagggcaccgggtacgttacctcgtgaggagccgagtccgcgggtcctggggttgagatgtggacgccctcagggctggcactctgccctggcggccacagtcatggaagtcccaacgcttctctcggctccgcaaccccagagggcggccacgaggagggcccgccacgcacgaccccagagggcggccaccaggagggcccgccacgcgcgaccccagagggcggccaccaggagggcccgccacggcgttgcggcagcagcccagaaggtgccctgcgcacggtccggacaggtgggatccgagttacctggccaagggggctgacgcagacacgtcgcgggacacagtgaagagtgtggtgcagagcggagggcgggagtctttggagaacaggtaggggcgtggggcacgcgcctcccacgcgcaggagccgtctaccgtggagggacacgggtggtcctgctggaggctcctctccgttagctgtctccatcgtctgattcttggatcccaggatggtgggatcatcagcaactgagatgaacccactgccccggccccctgagcccgcaggtccccacgccttgccagctgtgcccgagctggctgcaccccgggccaggcatccagcaaccttgagcagtggggtccggcttttcagaaggggccaggaacccgcgtggctgaggtgtgaccgaagcgtggggcagaggcgctgggccctggcgctttaacgctggtgtttctggttttaaatttcacgacccagtgacactgccaccctgctacctcgccagcagccctcctgggcttaacttcgggagagcagttttgctagccggccctgggtgccaagccctgcaggaggcgcagacccctggagacaggaccggactctgcagagcccgaccagcctcccagcttggccttttcctgacgcacgggcgcagaaggaaagccacagcaccggcttctctttgtaagtagtgtattttaaatagctttcaagatacacatattttttcctttaaaaaagtctgttggagcagttttgttcttgaattttgctggtcatcctcatggtcccgagcccccctactccgggtcgtggaggcggccgagggggaggctgggggcccacgtggcccgtcctggcggcacctgcagcactgggggagccgctgaaccccgtgcttcagcgctgggggagccgctgggccccgtcttccgccacaaaccatgcatggccgccacgtgagctcaaacgtccgtttatttcaaagcagtaataatttaaaattataaaaatctttccaccgctgaacgtttagagggtgaggttagacagaggacggggaggctggggacgccccagaggggaccatgtggcccacgccttcccaagccagggggccggtgggccgggcccgggtcctgccctggaacaggcgggacctgcagcgctgaccagccaagcgtggcgccgccggggcacccagtctgtgggtgccgtgtggcgctggctgagggtgggtgggaaaggccccgtgctttcccgacggccgacgtgggctcacgagttgcttgtggcgttctcgttgctgggcgagctggaggaggacgatgacgacgaggaggagaagctcaccccagtgaggccaggggggttcgtggccgtgttctgtcccgtgaggctttttcggcagacggggcagctgtcgtgctttgtggggacagaggcagggacgggagaaggggcaggttagaggcgggagggccgcggtcggggtgggggggcgggtgggcggggcactcacctgctccagccagggcacgatgcagccgtcgtggaacaggtggttgcagggcagctgccgcacacgctcacccagcgcgtagtcgtccttgcacacagggcactcgagcccggagcctgcgggagtgtgcagctgcggtcacagcgggcgtggggggcctgccgagccttcaagggcaggctactccacagcctcagccggaggccgcccctgagcccagcgaggggagaaaagccgtgtgtgtgtcccccgggctgccagaggggacctggacagaaccctctcctcccagcccaccttcagggaaatgctcgaggccgggtgcggtggctcacgcctgtcatcccagcactttgggaggccgaggcaggaggatcacctgaggtcaggagttcgagacctgcctgaccaacatggtgaaaccctgtctctactgaaaatacaagtatgagccaggcgtggcggcgggtgcctgtaattcccactactcgggaggctgagctctcatacctacgtgctcctcagtgacggggacggtggggagggcctggattttctctttatctgccggtggggggcctgtgttttcaaactgattgaggagctgaaagacaagaggcgagagtgccgggagctcctcgggggcccggcccggggctctgaaacgcgaggctgcaggacctgcaaaagcaccgaggccgcgtttgtcctgggccctgggccccttggagcccgcccggggtcggagatc

SEQ ID NO: 39 - DNA, 720 bp, Unknown
  Description of Unknown Organism:Unknown
cgccggcgcttgacctgactttcatgaatcgaaaaggaaatcctctatgaacgcactgcatcgcatcggcgccggaacgctactggccgtgttgctcgcttttggcctgaccggctgcggggagaaggaggaggttcagcagtcgctcgagccggtggcttttcacgactctgacgagtgtcacgtgtgcggcatgatcatcactgacttccccggccccaagggccaggcggtcgaaaagcggggagtgaagaaattttgttccaccgccgaaatgcttggttggtggctgcagccggaaaaccgtctgctcgatgccaagctctacgtccacgacatggggcgcagcgtttgggaaaagccggatgacggtcatctgatcgacgcaaccagcgcctactatgtggtcggtacgtcactcaaaggcgccatgggcgcgtcgcttgcaagctttgccgaggagcaggacgccaaggcgcttgccggcatgcacggcggtcgtgtgctgcgcttcgaggaaatcgatcaggcgctgctgcaggaggctgcaagcatgcagcacggcggcatgcacgaccatgcgccaaacggtgcacataacgcacacgcaggccactgagcagcagtggtctgaacagcacacacaagaaatcgaggtaagcacaatgatgggtatcagcgtctggcaactcctgatcattcttctgatcgtcgtcatgc

SEQ ID NO: 40 - DNA, 127 bp, Unknown
  Description of Unknown Organism:Unknown
  Misc_feature (9): N is any nucleic acid
  Misc_feature (101): N is any nucleic acid
  Misc_feature (119): N is any nucleic acid
gcggccgcncggcgctggctgctgtgcggaggccacggcgggccgcgagccgcctcgtcctcgccctcctgccctgggtgcggccccccgggtcccggcgncccactcgccccggcgtncccgcgct

SEQ ID NO: 41 - DNA, 6858 bp, Unknown
  Description of Unknown Organism:Unknown
actcgccaagtgatcgaccggcccctgagggccgcgacgcagagggcgccccgtgcactggcacaggcggccttgtgcgttagactctgatattcgtgcgccctctcgttggcaggaccatccatcctgtgtgccgggggccgcgcacaccgatcccggatccgcctcggccctgccctgcgcgcccctccgttctcgacctccccgacgctgtctgaacacgcgtcgccgggggacgacggcgggcggcccgcctcgggggaggggtaagcgtcccgggatgcccgttcaaccgttccgcaaggctcgcccatcgtgggggagaaccggcgcgacgctaggagagacaagtgatccagcaggagtcgcggctcaaggtcgccgacaacaccggtgcgaaggaaatcctgaccatccgtgtgctcggcggttccggacgccgctacgcaggcatcggcgacaccatcgtcgccaccgtgaaggacgccatccccggcggcaacgtcaagaaggcgcacgtcgtcaaggccgtggtggtccgcacccgcaagcagtcccgccgtcccgacggctcgtacatcaagttcgacgagaacgcggcggtcatcctgaagaccgacggcgagccccgtggcacgcgcatcttcggccccgtgggtcgcgagctgcgtgacaagaagttcatgaagatcgtgtcgctcgccccggaggtgatctgacctcatggccaagatcaagaaggacgacctcgtgcaggtcatcagtggcaaggacaagggcaagcagggcaaggtcctgcgcgtgttcccgacggatgagcgcgtgctcgtcgagggcgtgaaccgcgtgaccaagcacctgcgcgccggccaggacaacaacggttccaccgagggcggcctgcaggtcgtcgaggccccgatccacatctcgaacgtggccgtggtggacccggagaccaagaagccgacccgtgtgggctaccgcttcgagaccgtcgagaaggacggcgtgacgaagaccgtgaaggtccgcttcgccaaggcctcggggaaggagctgtgatgaccgaggtgcagcagaccgagaaggtcaccccgcgtctgaagaccaagtaccgcgaggagatccgcggacgcctgcaggagcagttccagtacgggaacgtcatgcaggtgccgggcctcgtgaaggtcgtcgtcaacatgggcgtcggcgaggccgccaaggactccaagatcatcgacgacgccgtcaccgacctcaccgccatcaccggccagaagccgatgatcaccaaggcccgcaagtccatcgcgcagttcaagctgcgtgagggcatgcccatcggcacgcacgccaccctccgtggcgatcgcatgtgggagttcctggaccgcctggtcacgctgccgctgccgcgcatccgtgacttccgcggcctgtccgaccgccagttcgacggcaacggcaactacaccttcggcctgtccgagcagaccgtgttccacgagatcgatcaggacaagatcgaccgcgtgcgcggcatggacatcaccgtggtgacgaccgccaagaacgacgacgagggccgcgcgctgctcaaggcgctgggcttcccgttcaagaccgaccagtaagacctccacgccacaggtcctccaccggtgaaccggtggcggaaaccacggcgagaaagggcgtgaagcacatgaccatgaccgatcccgtcgcagacatgctgacccgtctgcgcaacgcaaactcggcctaccacgacaccgtgtccatgccgtcctcgaagctgaagactcgcgtcgccgagatcctcaaggccgagggctacatccaggactggcgcgaggaggaggccgaggtcggcaagaagctgaccatcgacctgaagttcggcccgcagcgtgagcgtgcgatcgccggcctgcgccgcatctccaagccgggcctgcgcgtgtacgcgaagtccacgaacctgccccacgtgctgggcggcctcggcatcgccatcctgtccacctcctctggtctcctcacgaaccagcaggccgccaagaaggctggcgtgggcggagaagtcctcgcctacgtctggtgacgggcaagacggaagaaaggctgaactgacatgtctcgaatcggacgtctcccgatcaccatccccgccggcgtcgatgtgaccatcgacggcgaccgcgtctccgtgaagggccccaagggccccaagggtcagctcgagcactcgctgcccacgcccatcacggccaccctcgaggaggggcaggtcaccgtggcccgccccgacgacgagcgtgagtcccgctccctgcacggtctgacccgtaccctcatcagcaacatggtcgagggcgtgaccaacggcttctccaagcagctcgaggtcgtcggcaccggctaccgcgtgcaggccaagggccaggacctcgagttcgacctgggctactcccaccccgtcccggtgaaggtgtcccagggcatcaccttcacggtggagggtaacagggtcaccgtcgccggtatcgacaagcagcagcaggtcggcgagaccgccgccaacatccgcaagctgcgccgccccgacccgtacaagggcaagggcgtctacgcgggcgagcagatccgccgcaaggccggaaagaagtgatgtctactctgaaggtgaagggcaagggcaagttcaacgcccgcacccgccgccacctccgggtgcgcaagcggatctccggcaccacgtccgtcccccgcctcgtcgtcaaccgctctgcacggcacatgttcgtgcaggtcgtggacgacacgcagagccgcacgatcgcgtacgcctccaccatggaggccgacgtgcgtgcgctcgagggtgacaagacggccaaggccaagcgcgtgggcgagctcgtcgccgagcgtgccaaggcggccggcatcgaggccgcggtcttcgaccgggcgggcaacaagtaccacgggcgcgtcgcggccgtggccgacggtgcgcgagagggtgggctgcagctgtgaccgagaacatcaaccagaaggacactcaggtgaccgagagcaccgagaccaccgtctccgagaccgggtcgggctcgcgagccagaccaccgagcgcgccaccggtggccgcggcggtcgcgacggcggccgcggtggccggacggcgatcgtcgtggcggccgtcggacgaccgaaccgtcgtggcgcccaggacgacgaggaaggaccagttcctcgagcgcgtcgtgggcatcaaccgcgtctccaaggtcggccgccgcttctccttcaccgccctcgtggtggtgggtgacggcgacggcaccgtcggcgtcggctacggcaaggcgaaggaggtccccgctgcgatccagaaggccgtggaggaggccaagaagtccttcttccgcgtcccccgcgtcggctccaccatcccgcacctggtgcagggtgaggacgccgccggcgtcgtgctgctccgcccggcctccccgggtaccgcggtgatcgccggcggtccggtgcgcgccgtgctcgagtgcgccggcatccacgacgtgctctccaagtccatgggctccgtgaacgcgatcaacatcgtgcgcggcacggtggagggcctcaagaagctgaagagcccccaggccgtcgccgcccgccgcggcaaggccctggacgagatcgccccccatgcgatgctgcgcaccatggagaacgatcgcgcccagaagagcgcgaaggcaggtgcgtgacgcgtgtttgagtccactcgcaagaacatccagccctcggacgccaccctggtcatcacccagacccgcggcgtcacgggctccaagcagaaccatcgggacaccctgcgctcgctgggcctgaagcggatcggccaccaggtcacccgcaaggccgacgcggtgacggtcggcatggtcaacaccgtgccgcacctggtgtccgtggaggaggtcaacaatggctgacaacgacgccatcaaggtccacgacctgcgtccggcccccggtgccaagaccgccaagacccgcgtgggtcgcggtgaggcgtcgaagggcaagaccgccggtcgcggcaccaagggcaccaaggcccgttaccaggtccgtgcgggcttcgagggcggtcagctgcccctgcagatgcgtctgccgaagctccgcggcttcaagaacccgttccgcacggagtaccaggtcgtgaacctggacaagctctccgcgcacttccccgagggcggtgaggtcaccgtggacgcgctcgtctccaagggcctcgtccgtcgtggccagcccgtgaaggtgctgggcacgggggagatcaccgcggccgtgcaggtgaaggcgaacgccttctctgcgtccgccgtggagaagatccaggccgccggcgggtccaccgagaccctctgacacgccgacccatcgaccgagggccctggccggagcagccgctcgggccaggccctggtccgtccgtgtagactcgcacagccgccccggtgtggccgccgtctcgtgcccccgccccgcggaacggcgcacgccccacaggaccagccgcaggaggactcgtgctcaaggccatcgcccggatcgtccggacgcctgacctgttgcggaagatcgccttcacgctcgggctcatcgccgtctatcggatgggcgacttcgtgccggccaccggcgtggactacccggcggtgcagcagtgcctggcagcgggcaacgcccagggcggcctgtactccttcgtgaacatgttctcgggcggggcgctcctgcaggtgtctgtcttcgcgctgggcatcatgccgtacatcacggcgtcgatcatcgtgcagctgctgcgcgtggtgatcccgcgcttcgagcagctccaccaggagcgccgcaggggccaggcgacgctgacgcagtacacccgctacctgaccctcgccctcgccctgctgcaggcgaccacgatggcctcgctggcccgcaccggggccctgctcggatgcagcctgccgctgctgcgcgacggctccatcctcacggtgctgctcgtggtcatcgccctgaccaccggctgtctcatcgtcatgtggttcggggagcggatcaccgagaacggcgtgggcaacggcatgtccctgctcatcttcacctccatcgcggcaggcttcccggccggtctcggccaggtggtccagacgcagggctggcgcgtgttcgcgatcgtcatggggatcggcctgctcaccatgctggccatcgtcttcgtggaggagtcgcagcgccggatcccggtccagtacgccaagcggcagatcggctcacggaccgtgggcgggtcgagcacctacatcccggtcaaggtgaacatggccaacgtcatcccggtcatcttcgcctcctccgtgctgatgctcccgggcatcctcatccagttcaacacgccgcaggacggcagtgcgccggccccgtggatcacgtggctgagccggtacttcggctccggtgaccacccggtgtacatggccctgtacttcctgctcatcatcggcttcacgtacttctacgtgtccatcacgttcaacccggtggagatctcggacaacatgaagcgctacggcggcttcatcccggcgtccgcgccggccggccccaccgagcgttacctgcagtacgtcatcagccgcatcacgttcgtggtgggggccctctacctcggtatcgtggccatgatcccgctgatcgccttcgcggtgatcggcaccagccagaacttcccgctcggcggcacgtccatcctcatcatggtgggcgtcggcctccagaccgtgaagcaggtcagcgcacagatggagcagcgccactacgagggcctgctgcgctgagccccgacccgatcccgcaacgccgtccgtatcgacagtgaggaacacacgatgacccgcatgctgctcatgggccctcccggttccggcaagggcacccaggccacccggatcgccgacaagctggggatcgtcccgatctccaccggtgacatcttccgccacaacgtgaagtcgatgacgccgctcggcgtcgaggccaagaggtacatcgacaacggcgacttcgtccccgatgaggtcacgaaccgcatggtcgccgaccgcatcgcccaggccgacgcggagcacggcttcctgctggacggctacccgcgcacgaagggccaggtcgaggcgctggacgccatgctcgccgaggccggccagtcgctgtccgccgtcgtcgagctggaggtgcccgacgaggagctcgtggagcgcctgctcaagcgtgccgagatcgagggccgcgcggacgacacccaggaggtcatcgagcaccgcctggacctgtaccaccgcgagaccgagtccgtcatccaggagtacgtggagcgcggcatcgtcgcccgcgtggacggcaccggccagatcgacgacgtcaccgagcgcctgctgcaggccgtgtactccgtgcgctccgccacgggctccctgcccgtgatccagccgggcgcggagtcctgaccccgtgatcggccgccgctcgctcgagctcaagaccgccccccagctgctggccatgcagcgcgcgggggtggtcctgtccgaggcactggacgccgcgctggccggcgcgccgggcttcaccaccgcggagctggacgccgtgttcgcggtggtgctggccgaacgcggtgcgacctccaacttcctgggctactacgacttcccggcctcgatctgcacctcggtcaacgaggaggtcgtgcacggcatcccc

SEQ ID NO: 42 - DNA, 578 bp, Homo sapiens
  Misc_feature (5): N is any nucleic acid
  Misc_feature (23): N is any nucleic acid
  Misc_feature (31): N is any nucleic acid
  Misc_feature (48): N is any nucleic acid
  Misc_feature (211): N is any nucleic acid
  Misc_feature (292): N is any nucleic acid
  Misc_feature (308): N is any nucleic acid
  Misc_feature (350): N is any nucleic acid
  Misc_feature (384): N is any nucleic acid
  Misc_feature (477): N is any nucleic acid
  Misc_feature (507): N is any nucleic acid
  Misc_feature (529): N is any nucleic acid
  Misc_feature (549): N is any nucleic acid
  Misc_feature (551): N is any nucleic acid
  Misc_feature (558): N is any nucleic acid
ttctngtctatggcagagatggncaggttgncgttgagcaggtactgnccatcagccgtcttcagcgccaggtagttcccatcgttctgcacacccgggtggctccgctgcttcacgtcaatattagtggcaccagctgggatggtgacaatgtcattgtagccataattggtgggggtgagggacccggagaccttcctgcaggagttgnctttgcccccacacaccccgcatttgtccagcttccgaggcgagtccaccacatggtcacagccggccttgacacactggncacggacacagatggncagtgtttctggcccacacagggtgccatcaatcaccttggnctcgaacactttggaactcgctcctcccccgggntcgggaggaacaacttgcaggggtcccgggggggacaacccagcattcttgggggacccactgcaggaggattccccgtccatgtcaagtgtnattggtgggcattattcttctcacaattgntgctccctgaaggttttcccgncaaggggggattcccccccngntggaatnattggtacttgggtctccga

SEQ ID NO: 43 - DNA, 305 bp, Homo sapiens
  Misc_feature (128): N is any nucleic acid
  Misc_feature (146): N is any nucleic acid
catttaagtttgctagtcctttgcaaacagactgacgctgagtgtcctgtctgagtcaataagtgcacttttaccttttaacctatgccctctacttgaacccgagcaaggtccagtccactggacangttgatgatagggtctgncgccccataccctctcctcttcccccttaggaatttgtgcagtactggaggggttgcggcaatgggaggcctgggtgggccgtgctgccttgatatggccaagggacccagtcaccacagtggagacccttgtctgcacctcagtaccgcatgtccagg

SEQ ID NO: 44 - DNA, 333 bp, Homo sapiens
  Misc_feature (82): N is any nucleic acid
  Misc_feature (255): N is any nucleic acid
  Misc_feature (275): N is any nucleic acid
  Misc_feature (299): N is any nucleic acid
  Misc_feature (313): N is any nucleic acid
  Misc_feature (324): N is any nucleic acid
ggcacaggtgactttagcatgcagagcagcaaagagagagcaaccaccaacatcatccagccgctgctccacgcacagtggntgctgggggactggtctgagtgctctagcactgcggggccggctggcagaggcgaactgtagagtgcagggacccctccggtgcaggcctctgccacctgcaacaaggctctggaaacccgaggatgccaagccctggcagaaccagctgtgccccctgtgatttcagggggncaggggccattttgtgctcngggacatgcggtaatggaggttgncagacaaggtcttncattgtggtgnatgggttcc

SEQ ID NO: 45 - DNA, 102 bp, Unknown
  Description of Unknown Organism:Unknown
  Misc_feature (64): N is any nucleic acid
  Misc_feature (69): N is any nucleic acid
  Misc_feature (71): N is any nucleic acid
  Misc_feature (72): N is any nucleic acid
gcagcagcagcgcagcgcagagagagcagcagcagcagcagcagcagcagcagagcagatcntnctggnannaaaaaatcgcggcagcagctgctctagcag

SEQ ID NO: 46 - DNA, 123 bp, Unknown
  Description of Unknown Organism:Unknown
  Misc_feature (9): N is any nucleic acid
  Misc_feature (51): N is any nucleic acid
  Misc_feature (52): N is any nucleic acid
  Misc_feature (57): N is any nucleic acid
  Misc_feature (67): N is any nucleic acid
  Misc_feature (123): N is any nucleic acid
caggcaagncggcacgtaggagcagcagcagcagcagcagcagcagtaacnnagtcnacgagggggngcccgggacccaaggcgcccgaacagagaggcggagcacaatccactggtcggcgn

SEQ ID NO: 47 - DNA, 109 bp, Unknown
  Description of Unknown Organism:Unknown
  Misc_feature (87): N is any nucleic acid
  Misc_feature (95): N is any nucleic acid
  Misc_feature (102): N is any nucleic acid
  Misc_feature (106): N is any nucleic acid
  Misc_feature (107): N is any nucleic acid
ggcacgcaggagcagcagcagcagcagcagcagcagcagcagagagagagcagcagagagagagagcagcagagcagagcagagcanagtagagnagagcanagcnnac

SEQ ID NO: 48 - DNA, 293 bp, Homo sapiens
  Misc_feature (86): N is any nucleic acid
  Misc_feature (166): N is any nucleic acid
  Misc_feature (185): N is any nucleic acid
  Misc_feature (209): N is any nucleic acid
  Misc_feature (214): N is any nucleic acid
  Misc_feature (219): N is any nucleic acid
  Misc_feature (234): N is any nucleic acid
  Misc_feature (290): N is any nucleic acid
ggcacgagggggaaactgctccgcgcgcgccggggaggaggaaccgcccggtcctttagggtccgggcccggccgggcatggattnaatgcctgagcccgggtcccgctgtcttctgcttcttcccttgctgctgctgctgctgctgctgctgccggccccggagntgggcccgagccaggccgnagctgaggagaacgacttgggttngcctncccanaaaatgggaaggganttggggttaatcgaagtcattgggaccattttaaaaggggcttcctggattatagnctt

SEQ ID NO: 49 - DNA, 506 bp, Homo sapiens
  Misc_feature (283): N is any nucleic acid
  Misc_feature (342): N is any nucleic acid
  Misc_feature (356): N is any nucleic acid
  Misc_feature (362): N is any nucleic acid
  Misc_feature (364): N is any nucleic acid
  Misc_feature (368): N is any nucleic acid
  Misc_feature (429): N is any nucleic acid
  Misc_feature (454): N is any nucleic acid
  Misc_feature (461): N is any nucleic acid
aattcggcacgagcacccggccactgcagtcttctgccctgctggacagcagcagcagcagcagcagcagcagcagcagcagcagcaacagtaacagcagcagttcgtccggacccaacccttctacctcctttgagcccatcaaggcagaccccacaggtgttttggaactccccaaagagctgtcagaaatctttgatcccacacgagagtgcatgagctcggagctgctggaggagttgatgtcctcagaagtgtttgcccctctgctttcgtctttctncacccccgggagaccacgattatatctacaacctggacgagagtgaaggtgtttgtganctcttttgatgtgnctgttntnaacntttgactgacagggacatgccttttttggttgggacccagattttttgacttgggggtttncttgggacttttcaaccgaccctanagagttnagagcaaanaggttggtttttcggcttccttaacgaaagttttgg

SEQ ID NO: 50 - DNA, 419 bp, Homo sapiens
  Misc_feature (137): N is any nucleic acid
  Misc_feature (221): N is any nucleic acid
  Misc_feature (259): N is any nucleic acid
  Misc_feature (327): N is any nucleic acid
  Misc_feature (385): N is any nucleic acid
  Misc_feature (389): N is any nucleic acid
  Misc_feature (416): N is any nucleic acid
  Misc_feature (418): N is any nucleic acid
tttaagcaccaaaacttgtgttttaatgatgttggatggaaatctttcctaaatgtgtcatgcatgctcttgtctcccttaatggagagagtgtgacactgcttagcacttggatggcttggggtggtggttatgancagcagtctgtcacagctcagcgaggtgaagcctgtgggcgttttgctctgtgctgaatggctcagtggccctacaaagcggantcagctcttggtggctttctgttgtggtgggctgctgntgctgctgctgctgctgctgctgctgcccttgcctctaaaagaactcacttcctcttcctcctgctgncacctgtcttttggcttgtgggattggagtcatggggcccagatggagccttgctccntganttatgataggcccctcggtctcttttntnc

SEQ ID NO: 51 - DNA, 495 bp, Saccharomyces cerevisiae
  Misc_feature (177): N is any nucleic acid
  Misc_feature (322): N is any nucleic acid
  Misc_feature (328): N is any nucleic acid
  Misc_feature (342): N is any nucleic acid
  Misc_feature (368): N is any nucleic acid
  Misc_feature (371): N is any nucleic acid
  Misc_feature (375): N is any nucleic acid
  Misc_feature (380): N is any nucleic acid
  Misc_feature (386): N is any nucleic acid
  Misc_feature (396): N is any nucleic acid
  Misc_feature (404): N is any nucleic acid
  Misc_feature (423): N is any nucleic acid
  Misc_feature (426): N is any nucleic acid
  Misc_feature (436): N is any nucleic acid
  Misc_feature (443): N is any nucleic acid
  Misc_feature (456): N is any nucleic acid
  Misc_feature (460): N is any nucleic acid
  Misc_feature (467): N is any nucleic acid
  Misc_feature (468): N is any nucleic acid
  Misc_feature (471): N is any nucleic acid
  Misc_feature (474): N is any nucleic acid
aattcggcacgagcaaagttctgcgctccattgtgggcatcaaacgacacgtcaaagccctccatctgggggacacagtggactctgatcagttcaagcgggaggaggatttctactacacagaggtgcagctgaaggaggaatctgctgctgctgctgctgctgctgccgcagacncccagtccctgggactcccacctccgagccagctcccacccccagcatgactggcctgcctctgtctgctcttccaccacctcttgcacaaagcccagtcctccggcccagaacatcctgggcccggagttccttccttgccttnaggggnttttcagcaagttnagttccttgggtcctttttgggaaanttnaggnagttnaaggantaccaggttnttgccatnctttccagatccaagtttnacnaaaaattttnaacagtntaaattgggtttnttgnccctttnnggnggntgttttttttttcgggtccgg

SEQ ID NO: 52 - DNA, 81 bp, Unknown
  Description of Unknown Organism:Unknown
  Misc_feature (65): N is any nucleic acid
  Misc_feature (67): N is any nucleic acid
  Misc_feature (71): N is any nucleic acid
  Misc_feature (75): N is any nucleic acid
ggcacgcaggagcagagcagcagcagcagagagagcagcagcagcagcagcagcagcagagagananatanatanatatat

SEQ ID NO: 53 - DNA, 305 bp, Homo sapiens
  Misc_feature (11): N is any nucleic acid
  Misc_feature (62): N is any nucleic acid
  Misc_feature (81): N is any nucleic acid
  Misc_feature (256): N is any nucleic acid
  Misc_feature (289): N is any nucleic acid
aggcacttganttgaaaatggaaaaccctactgctggtggtgctgcggtgatgaggcctatnatgcagccccagggttttnttaatgctcaaatggtcgcccaacgcagcagagagctgctaagtcatcacttccgacaacagagggtggctataatgatgcagcagcagcagcagcagcaacagcagcagcagcagcagcagcagcagcaacagcaacagcaacagcaacagcagcaacagcagcaaacccaggncttcagcccacctcctaatgtgactgcttcccncagcatggatgggctt

SEQ ID NO: 54 - DNA, 307 bp, Hepatitis C virus
  Misc_feature (212): N is any nucleic acid
tggggtgtgaagctccggtgctggtgcggcgggggactgcggggccagcctcagtttaaaccccctcagcagtctttctgtcgttgccctccacactgcgagactctggagggcgatctggaggtctggaagataaccgattcctgggagatttgggggtagtctccaatctgtccctggctcatcttgtgacccgaagccggcggccttgncaggagtattctagaatgagtgcacataaaaataccttcaaacggtagcagcagcagcagcagcagcagcagcaagcagcagcagcagcagcagc

SEQ ID NO: 55 - DNA, 88 bp, Unknown
  Misc_feature (6): N is any nucleic acid
  Misc_feature (7): N is any nucleic acid
  Misc_feature (78): N is any nucleic acid
  Misc_feature (83): N is any nucleic acid
  Misc_feature (87): N is any nucleic acid; Description of Unknown Organism:Unknown
ggacanngactactctctctctctctctctctctctctgctgctgctgctgtgctgctgctgctgctgctgctgccgntgtgngcana

SEQ ID NO: 56 - DNA, 346 bp, Unknown
  Misc_feature (278): N is any nucleic acid
  Misc_feature (288): N is any nucleic acid
  Misc_feature (299): N is any nucleic acid
  Misc_feature (313): N is any nucleic acid
  Misc_feature (342): N is any nucleic acid; Description of Unknown Organism:Unknown
ggcacagcccaactggtgatgctgctgctgctgctgctgctgccgccgccgcctctattgctgatactctagtggggctggaagggtggttcctattcgcaccatcgccaaccagagacagagggaaaaaaaaaaccggcagccactgctgaatgttgggttcggaggctgcatccgactcggtcacaaggaaaatggattcagtttgcatctctccctcctttaaacagcttctccgggtctcagcatgggcttccagggcagcgattgaggagacnttaccaaggngcaccacacantagatgctgagacntcgtgactccaggataagaaacattaacngggg

SEQ ID NO: 57 - DNA, 496 bp, Unknown
  Description of Unknown Organism:Unknown
  Misc_feature (11): N is any nucleic acid
  Misc_feature (78): N is any nucleic acid
  Misc_feature (195): N is any nucleic acid
  Misc_feature (197): N is any nucleic acid
  Misc_feature (286): N is any nucleic acid
  Misc_feature (291): N is any nucleic acid
  Misc_feature (293): N is any nucleic acid
  Misc_feature (315): N is any nucleic acid
  Misc_feature (328): N is any nucleic acid
  Misc_feature (329): N is any nucleic acid
  Misc_feature (344): N is any nucleic acid
  Misc_feature (346): N is any nucleic acid
  Misc_feature (352): N is any nucleic acid
  Misc_feature (354): N is any nucleic acid
  Misc_feature (358): N is any nucleic acid
  Misc_feature (366): N is any nucleic acid
  Misc_feature (399): N is any nucleic acid
  Misc_feature (406): N is any nucleic acid
  Misc_feature (410): N is any nucleic acid
  Misc_feature (418): N is any nucleic acid
  Misc_feature (420): N is any nucleic acid
  Misc_feature (435): N is any nucleic acid
  Misc_feature (443): N is any nucleic acid
  Misc_feature (453): N is any nucleic acid
  Misc_feature (454): N is any nucleic acid
  Misc_feature (459): N is any nucleic acid
  Misc_feature (471): N is any nucleic acid
  Misc_feature (473): N is any nucleic acid
  Misc_feature (474): N is any nucleic acid
  Misc_feature (481): N is any nucleic acid
gaattcggcanaggtgcacagatgtggtggatggggagggccgcacgggacagaagttctccctgtgtattctgacgnctgagaaaggagcatttcatccgggcggagaccaaggagatcgtcaatgggtggctggagatgctcatggtctatccccggaccaacaagcagaatcagaagaagaaacggaaagtngnagccccccacaccacaggagcctgggactgccaagttgggctgttaccagcagcagcagcagcagcagcagcagcagcagcatccccantgctntnggaaagttcccaccaccaagtnccacaatttgggnnaaaaccaaggttgtngnagacgngntttngggatttnggcattgtgggttgcttgcatggaaggacattnggttgtnggtnccttggangntacaattaccatttncggttgtnaaggttaaanntccgncattcagaaggntnnaaggtgntttgaagtccatttg

SEQ ID NO: 58 - DNA, 268 bp, Drosophila sp.
  Misc_feature (16): N is any nucleic acid
  Misc_feature (51): N is any nucleic acid
  Misc_feature (60): N is any nucleic acid
  Misc_feature (202): N is any nucleic acid
aacacttatccttganagctctgtttgggaagcaggacaaagctacatgtnaggaaactntggagcctccgcagactctccaccagcagcagcagcagcagcagcagcagcaagagaagcttccaattaggcagggggttgtacgctccctgtcctatgaggaacccagaagacactcaccccccattgagaagcagctctntccagccattcagaaactcatggtcaggagcgcagacctccacccattgtcagagctgcctgaaaa

SEQ ID NO: 59 - DNA, 471 bp, Homo sapiens
  Misc_feature (249): N is any nucleic acid
  Misc_feature (386): N is any nucleic acid
  Misc_feature (449): N is any nucleic acid
tcgacccacgcgtccgctgaggaacagacgttccctggcggccctggcgccttcaaacccagacatgctgctgctgctgctgctgctgcccctgctctgggggacaaaggggatggagggagacagacaatatggggatggttacttgctgcaagtgcaggagctggtgacggtgcaggagggcctgtgtgtccatgtgccctgctccttctcctacccccaggatggctggactgactctgacccagntcatggctactggttccgggcaggagacagaccataccaagacgctccagtggccacaaacaacccagacagagaagtgcaggcagagacccagggccgattccaactccttggggacatttggagcaacgactgcnccctgagcatcagagacgccaggaagagggataaggggtcatatttctttcggctagagagangaagcatgaaatggagttacaa

SEQ ID NO: 60 - DNA, 379 bp, Unknown
  Description of Unknown Organism:Unknown
  Misc_feature (2): N is any nucleic acid
  Misc_feature (14): N is any nucleic acid
  Misc_feature (31): N is any nucleic acid
  Misc_feature (135): N is any nucleic acid
  Misc_feature (315): N is any nucleic acid
  Misc_feature (332): N is any nucleic acid
  Misc_feature (349): N is any nucleic acid
  Misc_feature (357): N is any nucleic acid
  Misc_feature (374): N is any nucleic acid
anttcggcanaggnaagggagagggtgaccngcatcccaactagatttcagtggagtgaagttcaggaggcatggagctgacaaccatgaggcctcggcagccaccgccaccaccgccgccgccaccaccgtagncagcagcagcagcagcagcagcagcaagagttaactctgacttagggaatagagacagccagagagaaatgtgatcaatgaaggagacatctggagtgtgcgtgcttcttcagaggggacgggtgatgggcagatttggaaaaagcaccgcagattgggaaccttatcttttctttttcntaaaattgttgttatgnaaatttgggtttttccngtaacttnttaaaaacttaaaagtnggttt

SEQ ID NO: 61 - DNA, 255 bp, Unknown
  Description of Unknown Organism:Unknown
  Misc_feature (121): N is any nucleic acid
  Misc_feature (183): N is any nucleic acid
  Misc_feature (254): N is any nucleic acid
  Misc_feature (255): N is any nucleic acid
aattccgacaatggaaagcactcttagccttgcagtggtctacatttttaaggaaccaatatttcagcattctttattacccggcacgctgtgtcctttgtcagagttcaagtttatggtnactgccagggtcagacagtccatttgctgctgctgctgctgctgctgctttctcgaactggnatggcattagggaagctgctgtctgagtgttagggaatgtcttggctaagtaaagccaatgttctttcctnn

SEQ ID NO: 62 - DNA, 5289 bp, Unknown
  Description of Unknown Organism:Unknown
cgagctctcccagccgcagcctccgaatccacggcctccaccccgcgcctctccagcgctctatcccgtcgctgcgcccttgtcgccggccccggccgctgcatccgcgtccgcacaggctccttgctgggcacaaatagctccaccatggggctggcctggggactcggtgtcctgctcctgttgcatgcctgcggctccaaccgcattccagagtctgggggagacaacagtgtgtttgacatctttgaactcaccggagctgcccgcaagcggtctgggcgccgactggtgaagggccctgacccttctagcccagctttccgcatcgaggatgccaacctgatcccccctgtgcctgacaagaagttccaagacctagtggatgctgtgcgggcggagaaaggtttcctcctcctggcctccctgaggcaaatgaagaagacccggggtaccctgctggctgtggagcggaaagaccactctggccaggtcttcagcgtgatctccaatggcaaggcgggcaccctggacctgagcctgaccgtgcaggggaagcagcatgtggtgtcggtggaagaagcactcctggcgactggccagtggaagagcatcaccctgtttgtgcaggaggacagggcccagctgtacatcgactgtgagaagatggagaatgcggagctggatgtccccatccagagcatcttcaccagggacctggccagcatcgccaggctccgcattgccaaaggaggtgtcaacgacaatttccagggggtgctgcagaatgtaaggtttgtctttggaaccacaccagaagacatcctcaggaacaaaggctgctccagctctaccagtgtctttgtcacccttgacaacaacgtggtgaatgggtccagccctgccatccgcaccgactacattggccacaagacaaaggacctgcaagccatctgtggcatctcatgtgacgagctgtccagcatggtcctggagctcaggggtctacgcaccatcgtgaccacgctgcaggacagtatccgcaaagtgaccgaagagaacaaagagctggccaacgagctgaggaggcccccactctgctaccacaacggagtgcagtacaggactggcgacgagtggacggtggacagctgcactgagtgtcgctgccagaactcagttaccatctgcaaaaaagtgtcctgtcccatcatgccctgctccaatgccacagttccggatggagaatgctgcccacggtgctggcccagcgactctgcagacgatggctggtccccgtggtctgagtggacctcttgctctgtgacctgtggcaatggaatccagcagcgtggccgctcctgcgacagcctcaacaacagatgcgagggctcctctgtgcagacgcggacctgccacatccaggagtgtgacaagagatttaaacaggatggcggctggagccactggtccccatggtcatcttgctccgtaacatgtggagacggtgtgatcacaaggatccggctctgcaactcccccagcccccagatgaatgggaagccatgtgagggcaaagcccgggagaccaaagcctgccagaaagactcctgccccatcaatggaggctggggaccttggtcaccatgggacatctgttctgtcacctgtggaggaggggtacagaaacgtagccggctctgcaacaaccccaaaccccagtttggaggcaaggactgcgttggtgatgtgacagaaaaccagatctgcaacaagcaggactgtcccattgacggatgcctgtccaatccctgctttgctggtgtccagtgtaccagctaccctgatggcagctggaagtgtggtgcctgtcccccaggctatagtggagatggagtcgagtgcaaagacgttgatgagtgcaaagaagtccctgatgcctgcttcaaccacaatggagagcacaggtgtgagaacacagaccccggctacaactgcctgccctgcccaccgcgcttcactggctcgcagccctttggccggggcgtggaacatgccaccgccaacaagcaggtatgcaagccccgaaacccctgcacagacgggacacacgactgcaacaagaacgccaagtgcaactacctgggccactacagcgaccccatgtaccgctgcgagtgcaagcctggctacgccggcaacggcatcatctgcggggaggacacagacctggacggctggcccaatgaggacctgctgtgcgtggccaacgcaacttaccactgcagaaaggataattgccccaaccttcccaactcagggcaggaagactatgacaaggatggaatcggcgatgcctgcgatgatgacgatgacaatgataagattccagatgacagggacaactgtccattccattacaacccagcccagtacgactatgacagagatgacgtgggagaccgctgtgacaactgcccctacaaccacaacccagaccaggctgacacagataacaatggggaaggagacgcctgtgcagctgacattgatggggacagtatcctcaatgaacgggacaactgccagtatgtctacaatgtggaccagaaagacactgacatggacggggttggtgatcagtgtgacaactgccccctggaacacaatccagaccagctcgactctgactcggaccgcattggagacacctgtgacaacaatcaggatattgatgaagacggccaccagaacaatctggacaactgtccctacgtgcccaacgccaaccaggctgaccatgacaaggatggcaaaggcgatgcctgtgaccatgatgacgacaatgatggcattcctgatgaccgggacaactgcaggctggtgcccaatcctgaccagaaggactctgatggtgatggtcgaggtgatgcttgcaaagatgattttgaccaggacaaggtgccagacattgatgacatctgtcccgaaaatgttgatatcagtgagactgatttccgccgattccagatgattcctctagatcccaaagggacatcccagaatgaccctaactgggttgtacgccatcagggtaaagaactcgtccagactgtcaactgtgaccctggacttgctgtaggttatgacgaatttaacgccgtggacttcagtggcaccttcttcatcaacaccgagagggatgacgactatgccggctttgtgtttggctaccagtccagcagccgcttctatgttgtgatgtggaagcaagtcactcagtcctactgggacaccaaccccacgagggctcaggggtactctggactttccgtgaaggttgtaaactccaccacggggcctggcgagcacctgcggaatgccctgtggcacacaggaaacacctctggccaggtgcgcacactgtggcatgaccctcgtcacattggctggaaagatttcactgcctacagatggcatctgagccacaggccaaagacaggtttcatcagagtggtaatgtatgaagggaagaaaatcatggctgactcaggacccatctatgacaaaacctatgctggtgggaggctaggcttgttcgtcttctctcaagaaatggtgttcttctccgacctgaaatatgaatgcagagactcctaatcatcaaactgttgatcaaaagactgatcataaaccaatgctggtattgcaccttctggaaccatgggcttagaaaacccccaggatcgcgcctcgctgcctgcctttgctctctgcttgcatgagtgtggactcctagaacatgtgacttgcctcaagaaaatgcaattttccaaatcagaccctgcattcagcctctgactgagaagaatcttccaaggagacaaacaatgactttggttggcttttgcaaaagcaaaagcatccacatgctttggttggaaggtgcctgtcccactctgcttttgtcagagcagaatgcgactgtgaggccagctctgagcagtggactccaaaatgttttcaggcatgtgagagaagggaggactcactagaattgacaaacaaaaccagccctgacctactccctctggaatgggggcgggtgggggggccaaagcccaaaggggaggatgcatacccaagagatgattgtatgaagaaaatatggaggaactgttacatttttggtactaaatcattttcaggggattgaaagactattgctggatttcatgatgctgaccggtgttagctgattaacccacataaataggcacttaaataggagcagggaaggaaggaaaagactggcttctggacttcctcccagatttccaccccttaacacatcacctgtagtgaccagaacagggagtcggagttaaaccgacacaaggcagggccagctgctgcagcttggttctattgaaattgtcagttgtattccagatgtagcttctgcagatgtagcagcaaaataagaatacccaccatctcagcgagcaccaggctgtctcccaagggacggcagccatgcttgtatttttatggttagaaaggcacaaaattatcaactaagacattccttctttctctttttttcctgaacatcatggagttttccagttgtctcttttggactgtagtttttagtgttttaaacaaacactttacaatgtaaactatttattttttacttattctgggggatctgtctgaaagactattcatggaacaggaagaagcgtaaggactatccatatcatctttgctacaagtcattatgactgtaagattgtaaatacagattatttattaactctgttctacctggaatctagtttcatatggaaagtgtttgagagcaggtagttgagatcgatcagcaaatctttcacaggaatggcacaaggaaaccagcatagcaagctgctcttcaccttgtgcttagactggatgatttggaattcttttttccttttttttcccaagtggaattacttggttgtccatttgcaagtgtttttagtttgcaaagaaagccaagaggccattaatactgtcttatcccatcccttgtgcctatttccagggagatgaaaagcatctacatttattatttttgcctttttccaaaagaaaaaaatgacaaaggtgaaacttgtatacaaatattacctcatttgttgtgtgactgagtaaagaattttgggatcaaacagaaagagtttaagtgtctaacaaacttaaagctactgtagtacctaaaaaaaaaaaaaaaaaaaa

SEQ ID NO: 63 - DNA, 2053 bp, Unknown
  Description of Unknown Organism:Unknown
gaattccggcggccgctgagagcccaccctggcgagctctcccagccgcagcctccgaatccacggcctccaccccgcgcctctccagcgctctatcccgtcgctgcgcccttgtcgccggccccggcgctgcatccgcgtccgcacaggctccttgactgggcacaaatagctccaccatggggctggcctggggactcggtgtcctgctcctgttgcatgcctgcggctccaaccgcattccagagtctgggggagacaacagtgtgtttgacatctttgaactcaccggagctgcccgcaacggtactgggcgccgactggtgaagggccctgacccttctagcccagctttccgcatcgaggatgccaacctgatcccccctgtgcctgacaagaagttccaagacctagtggatgctgtgcgggcggagaaaggtttcctcctcctggcctccctgaggcaaatgaagaagacccggggtaccctgctggctgtggagcggaaagaccactctggccaggtcttcagcgtgatctccaatggcaaggcgggcaccctggacctgagcctgaccgtgcaggggaagcagcatgtggtgtcggtggaagaagcactcctggcgactggccagtggaagagcatcaccctgtttgtgcaggaggacagggcccagctgtacatcgactgtgagaagatggagaatgcggagctggatgtccccatccagagcatcttcaccagggacctggccagcatcgccaggctccgcattgccaaaggaggtgtcaacgacaatttccagggggtcctgcagaatgtaaggtttgtctttggaaccacaccagaagacatcctcaggaacaaaggctgctccagctctaccagtgtctttgtcacccttgacaacaacgtggtgaatgggtccagccctgccatccgcaccgactacattggccacaagacaaaggacctgcaagccatctgtggcatctcatgtgacgagctgtccagcatggtcctggagctcaggggtctacgcaccatcgtgaccacgctgcaggacagtatccgcaaagtgaccgaagagaacaaagagctggccaacgagctgaggaggcccccactctgctaccacaacggagtgcagtacaggactggcgacgagtggacggtggacagctgcactgagtgtcgctgccagaactcagttaccatctgcaaaaaagtgtcctgtcccatcatgccctgctccaatgccacagttccggatggagaatgctgcccacggtgctggcccagcgactctgcagacgacggctggtccccgtggtctgagtggacctcttgctctgtgacctgtggcaatggaatccagcagctggccgctcctgcgacagcctcaacaacagatgcgagggctcctctgtgcagacgcggacctgccacatccaggagtgtgacaagagatttaaacaggatggcggctggagccactggtccccatggtcatcttgctccgtaacatgtggagacggtgtgatcacaaggatccggctctgcaactcccccagcccccagatgaatgggaagccatgtgagggcaaagcccgggagaccaaagcctgccagaaagactcctgccccatcaatggaggctggggaccttggtcaccatgggacatctgttctgtcacctgtggaggaggggtacagaaacgtagccggctctgcaacaaccccacaccccagtttggaggcaaggactgcattggtgatgtgacagaaaaccagatctgcaacaagcaggactgtcccattgacggatgcctgtccaatccctgctttgctggtgtccagtgtaccagctaccctgatggcagctggaagtgtggtgcctgtcccccaggctatagtggagatggagtcgagtgcaaagacgttgatgagtgcaaagaagtccctgatgcctgcttcaaccacaatggagagcacaggtgtgagaacacagaccccggctacaactgcctgccctgcccaccgcccggaatt

SEQ ID NO: 64 - DNA, 4339 bp, Unknown
  Description of Unknown Organism:Unknown
agccactgcctggagtcagccagcctcatcggacttctgcaggcaatcgcgaagctgctatccagttctgccacggtctctcccggcgcaccggcagtctcagcgtcttcaccggactcagcgtccttgtccttcacttcacctttgccacctctccgggttactgagccccggtgcacacaggctccgtgttgggcacaaaggctccaccatggagctcctgcggggactaggtgtcctgttcctgttgcatatgtgtggaagcaaccgcattccagagtctgggggagataacggtgtgtttgacatctttgaactcattggaggtgcacgaaggggccccggtcgccgactggtgaagggccaagatctatccagccccgccttccgcattgagaatgccaacctgatccccgctgtgccggatgacaagttccaagacctactggacgctgtgtgggccgacaaaggcttcatcttcttggcttccttgaggcagatgaagaagacccggggcacactcctggctgtggaacggaaagacaacactggccagatcttcagtgtggtctccaacggcaaagctggcaccctggacctgagcctgagcctgccagggaagcaacaagtggtgtcagtggaggaagctctcctggccactggccagtggaagagcatcacgctgtttgttcaagaggaccgggctcaactctacatagactgtgataagatggagagcgcggagctggatgtacccatccagagcatcttcaccagggatctggccagcgttgccaggctccgagttgcaaagggagatgtcaatgacaattttcagggggtgctgcagaatgtgaggtttgtctttggaaccaccccagaagacattctcaggaacaaaggctgctccagctctaccaacgtccttcttacccttgacaacaacgtggtgaacggttccagccctgctatccgcaccaactacatcggccacaaaacaaaggacctccaagctatctgtggcctctcctgtgatgaactatccagcatggtcctggaactgaagggcctgcgcaccatcgtgaccactctgcaggacagcatccgaaaagtgacggaagagaacagagagctggtcagtgagctgaagcggcctcccctctgctttcacaatggagtccagtacaagaacaacgaggagtggactgtagacagttgcacagagtgtcactgccagaactcggttaccatctgcaaaaaggtgtcctgtcccatcatgccctgctccaacgccacagttcctgatggtgaatgctgcccacggtgctggcccagcgactctgctgacgatggctggtctccctggtctgagtggacctcctgctctgccacatgtggcaatggaattcagcaacgtggtcgttcctgtgacagcctcaacaacagatgcgagggctcttcggtacagacgaggacctgccacattcaggagtgtgacaaaagatttaaacaggatggtggctggagtcactggtctccatggtcgtcctgttctgtgacctgtggtgacggtgtgatcacaaggatccgtctctgcaactcccccagcccccagatgaacgggaagccctgtgaaggtgaagcccgggagaccaaagcctgcaagaaagacgcctgcccaattaatggaggctggggtccctggtcaccatgggacatctgctctgtcacctgtggaggaggagtgcagagacgcagccgactctgtaacaaccccacaccccagtttggaggcaaagactgtgttggcgatgtgacagaaaatcaagtttgcaacaagcaggactgcccaattgatggatgcctgtccaatccctgctttgctggtgccaagtgtactagctaccctgatggtagctggaaatgtggtgcgtgtcctcctggctacagtggaaatggcatccagtgcaaagacgtcgatgagtgcaaagaagtgcctgatgcttgcttcaatcacaacggagaacatcggtgcaagaacacagatcctggctacaactgcctgccctgcccaccacgattcactggctcacagcccttcggccgaggtgtcgaacatgccatggccaacaaacaggtgtgcaaaccgcgaaacccctgcacggacgggacgcatgactgcaacaagaacgctaagtgcaactacctgggtcactacagcgaccccatgtaccgctgtgagtgcaagcccggctatgcaggcaatggcatcatctgcggagaggacacagacctggacggctggcctaatgaaaacctggtgtgtgtggccaacgcaacctaccactgcaaaaaggacaactgccccaaccttcccaactcggggcaggaagactatgacaaggacgggattggcgatgcctgcgatgatgacgatgacaacgacaagatccctgatgacagggacaactgtccattccattacaacccagcccagtatgactatgacagagatgatgtgggagaccgctgtgacaactgcccctacaaccacaaccctgaccaagcagacacagacaaaaacggggagggcgatgcctgtgctgtggacatcgatggagatggaatcctcaatgaacgagacaactgccagtacgtttacaacgtggaccagagggacacggacatggatggggttggagatcagtgtgacaactgccccctggaacacaatccagaccagctggactctgactcagacctcataggggacacttgtgacaacaatcaggacatcgatgaggatggccatcagaacaacctggacaactgtccctatgtgcctaacgccaaccaggccgaccatgataaagatggcaaaggagatgcctgtgaccatgacgatgacaatgacggcatccctgatgacagagacaactgcaggctggtgcccaatcctgaccagaaggactctgatggtgatggccgaggtgacgcctgcaaagacgactttgaccatgacaatgtgccagatattgatgacatctgtcctgagaattttgacatcagtgaaaccgatttccgacgattccagatgattcctctagatcccaaaggaacctcccaaaatgaccctaactgggttgtccgccatcagggcaaagaactcgtccagactgtaaactgtgaccctggacttgctgtaggttatgatgagtttaatgctgtggacttcagcggtaccttcttcatcaacaccgagagagatgatgactacgctggcttggtattcggctaccagtccagcagccgcttctacgttgtgatgtggaaacaagtcacccagtcctactgggacaccaaccccacaagggctcagggatactcaggcctgtctgtaaaggttgtgaactccaccaccggccctggcgagcacctgcggaatgcactgtggcacacaggaaacacccctggccaggtgcgcaccctgtggcatgaccctcgccacatcggctggaaagatttcactgcgtacagatggcgtctcagccacaggccaaagaccggttatatcagagtggtgatgtatgaaggaaagaaaatcatggctgactcgggacccatctatgacaaaacctacgccggcggtagactaggcctgttcgtcttctctcaggaaatggtgttcttctcagacatgaaatacgagtgtcgagattcctaatcatcagctgccaatcataaccagcgctggcaatgcaccttctaaaaacaagggctagagaaaccccccacccctgccgggatcgcctttcctcgccttccttgcctctcttcttgcatagtgtggacttgtaaagcctgagacctgcctcaagaaaatgcagttttcgaacccagagtcagcactcggcctttaacgaatgagaatgcatcttccaagaccatgaagagttccttgggtttgcttttgggaaagccaaagcgcctatttacttcccactaggaaggtgcccgctccactctgccttactcacagagccagaacttcttcgaggccacctctgagcagcacacacagaagcattttcaggcatgtcaaagaaaggaaaaatgactcactagaactcaccgccaaacaacctctgacataggtcctgagatgtggggaggcaggagccaaagctctagggagggcatgtacccaagagatgactgtatgaagaaaatgtggaggagctgttcggtactaaatcattttcaggggacagacagacttgctgcatttccgcatgctgctggtgagagctgattgacccaatcttccacacaggcactt

SEQ ID NO: 65 - DNA, 186 bp, Unknown
  Description of Unknown Organism:Unknown
gcacagttaatggaggctggggtccctggtcaccatgggacatctgctctgtcacctgtggaggaggagtgcagagacgcagccgactctgtaacaaccccacaccccagtttggaggcaaagactgtgttggcgatgtgacagaaaatcaagtttgcaacaagcaggactgcccaattggtaagc

SEQ ID NO: 66 - DNA, 5774 bp, Unknown
  Description of Unknown Organism:Unknown
gtcactttggttgatagcagccgctctggtagaggttaggacttcagctgatggacaagctggtaatgaagaaatggtgcaaatagatttaccaataaagagatatagagagtatgagctggtgactccagtcagcacaaatctagaaggacgctatctctcccatactctttctgcgagtcacaaaaagaggtcagcgagggacgtgtcttccaaccctgagcagttgttctttaacatcacggcatttggaaaagattttcatctgcgactaaagcccaacactcaactagtagctcctggggctgttgtggagtggcatgagacatctctggtgcctgggaatataaccgatcccattaacaaccatcaaccaggaagtgctacgtatagaatccggaaaacagagcctttgcagactaactgtgcttatgttggtgacatcgtggacattccaggaacctctgttgccatcagcaactgtgatggtctggctggaatgataaaaagtgataatgaagagtatttcattgaacccttggaaagaggtaaacagatggaggaagaaaaaggaaggattcatgttgtctacaagagatcagctgtagaacaggctcccatagacatgtccaaagacttccactacagagagtcggacctggaaggccttgatgatctaggtactgtttatggcaacatccaccagcagctgaatgaaacaatgagacgccgcagacacgcgggagaaaacgattacaatatcgaggtactgctgggagtggatgactctgtggtccgtttccatggcaaagagcacgtccaaaactacctcctgaccctaatgaacattgtgaatgaaatttaccatgatgagtccctcggagtgcatataaatgtggtcctggtgcgcatgataatgctgggatatgcaaagtccatcagcctcatagaaaggggaaacccatccagaagcttggagaatgtgtgtcgctgggcgtcccaacagcaaagatctgatctcaaccactctgaacaccatgaccatgcaatttttttaaccaggcaagactttggacctgctggaatgcaaggatatgctccagtcaccggcatgtgtcatccagtgagaagttgtaccctgaatcatgaggatggtttttcatctgcttttgtagtagcccatgaaacgggccatgtgttgggaatggagcatgatggacaaggcaacaggtgtggtgatgagactgctatgggaagtgtcatggctcccttggtacaagcagcattccatcgttaccactggtcccgatgcagtggtcaagaactgaaaagatatatccattcctatgactgtctccttgatgacccttttgatcatgattggcctaaactcccagaacttcctggaatcaattattctatggatgagcaatgtcgttttgattttggtgttggctataaaatgtgcaccgcgttccgaacctttgacccatgtaaacagctgtggtgtagccatcctgataatccctacttttgtaagactaaaaagggacctccacttgatgggactgaatgtgctgctggaaaatggtgctataagggtcattgcatgtggaagaatgctaatcagcaaaaacaagatggcaattgggggtcatggactaaatttggctcctgttctcggacatgtggaactggtgttcgtttcagaacacgccagtgcaataatcccatgcccatcaatggtggtcaggattgtcctggtgttaattttgagtaccagctttgtaacacagaagaatgccaaaaacactttgaggacttcagagcacagcagtgtcagcagcgaaactcccactttgaataccagaataccaaacaccactggttgccatatgaacatcctgaccccaagaaaagatgccacctttactgtcagtccaaggagactggagatgttgcttacatgaaacaactggtgcatgatggaacgcactgttcttacaaagatccatatagcatatgtgtgcgaggagagtgtgtgaaagtgggctgtgataaagaaattggttctaataaggttgaggataagtgtggtgtctgtggaggagataattcccactgccgaaccgtgaaggggacatttaccagaactcccaggaagcttgggtaccttaagatgtttgatataccccctggggctagacatgtgttaatccaagaagacgaggcttctcctcatattcttgctattaagaaccaggctacaggccattatattttaaatggcaaaggggaggaagccaagtcgcggaccttcatagatcttggtgtggagtgggattataacattgaagatgacattgaaagtcttcacaccgatggacctttacatgatcctgttattgttttgattatacctcaagaaaatgatacccgctctagcctgacatataagtacatcatccatgaagactctgtacctacaatcaacagcaacaatgtcatccaggaagaattagatacttttgagtgggctttgaagagctggtctcaggtttccaaaccctgtggtggaggtttccagtacactaaatatggatgccgtaggaaaagtgataataaaatggtccatcgcagcttctgtgaggccaacaaaaagccgaaacctattagacgaatgtgcaatattcaagagtgtacacatccactctgggtagcagaagaatgggaacactgcaccaaaacctgtggaagttctggctatcagcttcgcactgtacgctgccttcagccactccttgatggcaccaaccgctctgtgcacagcaaatactgcatgggtgaccgtcccgagagccgccggccctgtaacagagtgccctgccctgcacagtggaaaacaggaccctggagtgagtgttcagtgacctgcggtgaaggaacggaggtgaggcaggtcctctgcagggctggggaccactgtgatggtgaaaagcctgagtcggtcagagcctgtcaactgcctccttgtaatgatgaaccatgtttgggagacaagtccatattctgtcaaatggaagtgttggcacgatactgctccataccaggttataacaagttatgttgtgagtcctgcagcaagcgcagtagcaccctgccaccaccataccttctagaagctgctgaaactcatgatgatgtcatctctaaccctagtgacctccctagatctctagtgatgcctacatctttggttccttatcattcagagacccctgcaaagaagatgtctttgagtagcatctcttcagtgggaggtccaaatgcatatgctgctttcaggccaaacagtaaacctgatggtgctaatttacgccagaggagtgctcagcaagcaggaagtaagactgtgagactggtcaccgtaccatcctccccacccaccaagagggtccacctcagttcagcttcacaaatggctgctgcttccttctttgcagccagtgattcaataggtgcttcttctcaggcaagaacctcaaagaaagatggaaagatcattgacaacagacgtccgacaagatcatccaccttagaaagatgagaaagtgaaccaaaaaggctagaaaccagaggaaaacctggacaacctctctcttcccatggtgcatatgcttgtttaaagtggaaatctctatagatcgtcagctcattttatctgtaattggaagaacagaaagtgctggctcactttctagttgctttcatcctccttttgttctgcattgactcatttaccagaattcattggaagaaatcaccaaagattattacaaaagaaaaatatgttgctaagattgtgttggtcgctctctgaagcagaaaagggactggaaccaattgtgcatatcagctgactttttgtttgttttagaaaagttacagtaaaaattaaaaagagataccaatggtttacactttaacaagaaattttggatatggaacaaagaattcttagacttgtattcctatttatctatattagaaatattgtatgagcaaatttgcagctgttgtgtaaatactgtatattgcaaaaatcagtattattttaagagatgtgttctcaaatgattgtttactatattacatttctggatgttctaggtgcctgtcgttgagtattgccttgtttgacattctataggttaattttcaaagcagagtattacaaaagagaagttagaattacagctactgacaatataaagggttttgttgaatcaacaatgtgatacgtaaattatagaaaaagaaaagaaacacaaaagctatagatatacagatatcagcttacctattgccttctatacttataatttaaaggattggtgtcttagtacacttgtggtcacagggatcaacgaatagtaaataatgaactcgtgcaagacaaaactgaaaccctctttccaggacctcagtaggcaccgttgaggtgtcctttgtttttgtgtgtgtgtgttcttttttaattttcgcattgttgacagatacaaacagttatactcaatgtactgtaataatcgcaaaggaaaaagttttgggataacttatttgtatgttggtagctgagaaaaatatcatcagtctagaattgatatttgagtatagtagagctttggggctttgaaggcaggttcaagaaagcatatgtcgatggttgagatatttattttccatatggttcatgttcaaatgttcacaaccacaatgcatctgactgcaataatgtgctaataatttatgtcagtagtcaccttgctcacagcaaagccagaaatgctctctccagggagtagatgtaaagtacttgtacatagaattcagaactgaagatatttattaaaagttgatttttttttcttgatagtatttttatgtactaaatatttacactaatatcaattacatattttggtaaactagagagacataattagagatgcatgctttgttctgtgcatagagacctttaagcaaactactacagccaactcaaaagctaaaactgaacaaatttgatgttatgcaaacatcttgcatttttagtagttgatattaagttgatgacttgtttcccttcaaggaaacattaaattgtatggactcagctagctgttcaatgaaattgtgaattagaaacatttttaaaagtttttgaaagagataagtgcatcatgaattacatgtacatgagaggagatagtgatatcagcataatgattttgaggtcagtacctgagctgtctaaaaatatattatacaaactaaaatgtagatgaattaacctctcaaagcacagaatgtgcaagaacttttgcattttaatcgttgtaaactaacagcttaaactattgactctatacctctaaagaattgctgctactttgtgcaagaactttgaaggtcaaattaggcaaattccagatagtaaaacaatccctaagccttaagtcttttttttttcctaaaaattcccatagaataaaattctctctagtttacttgtgtgtgcatacatctcatccacaggggaagataaagatggtcacacaaacagtttccataaagatgtacatattcattatacttctgacctttgggctttcttttctactaagctaaaaattcctttttatcaaagtgtacactactgatgctgtttgttgtactgagagcacgtaccaataaaaatgttaacaaaatat

SEQ ID NO: 67 - DNA, 5535 bp, Unknown
  Description of Unknown Organism:Unknown
ggactttagaagccgttgctgccctctctgtcacctgaagcggggccctctcccatcccacccttgccccgcctccctgcccccaccgggccggccctgcccgccgccggaccctggcatgtcaagacctggtccgcgcctgcctgcccagcccgcggaaccccggcggccccgcgagctaggatgaggggccaggccgccgccccgggccccgtctggatcctcgccccgctgctactgctgctgctgctgctgggacgccgcgcgcgggcggccgccggagcagacgcggggcccgggcccgagccgtgcgccacgctggtgcagggaaagttcttcggctacttctccgcggccgccgtgttcccggccaacgcctcgcgctgctcctggacgctacgcaacccggacccgcggcgctacactctctacatgaaggtggccaaggcgcccgtgccctgcagcggccccggccgcgtgcgcacctaccagttcgactccttcctcgagtccacgcgcacctacctgggcgtggagagcttcgacgaggtgctgcggctctgcgacccctccgcacccctggccttcctgcaggccagcaagcagttcctgcagatgcggcgccagcagccgccccagcacgacgggctccggccccgggccgggccgccgggccccaccgacgacttctccgtggagtacctggtggtggggaaccgcaaccccagccgtgccgcctgccagatgctgtgccgctggctggacgcgtgtctggccggtagtcgcagctcgcacccctgcgggatcatgcagaccccctgcgcctgcctgggcggcgaggcgggcggccctgccgcgggacccctggccccccgcggggatgtctgcttgagagatgcggtggctggtggccctgaaaactgcctcaccagcctgacccaggaccggggcgggcacggcgccacaggcggctggaagctgtggtccctgtggggcgaatgcacgcgggactgcgggggaggcctccagacgcggacgcgcacctgcctgcccgcgccgggcgtggagggcggcggctgcgagggggtgctggaggagggtcgccagtgcaaccgcgaggcctgcggccccgctgggcgcaccagctcccggagccagtccctgcggtccacagatgcccggcggcgcgaggagctgggggacgagctgcagcagtttgggttcccagccccccagaccggtgacccagcagccgaggagtggtccccgtggagcgtgtgctccagcacctgcggcgagggctggcagacccgcacgcgcttctgcgtgtcctcctcctacagcacgcagtgcagcggacccctgcgcgagcagcggctgtgcaacaactctgccgtgtgcccagtgcatggtgcctgggatgagtggtcgccctggagcctctgctccagcacctgtggccgtggctttcgggatcgcacgcgcacctgcaggcccccccagtttgggggcaacccctgtgagggccctgagaagcaaaccaagttctgcaacattgccctgtgccctggccgggcagtggatggaaactggaatgagtggtcgagctggagcgcctgctccgccagctgctcccagggccgacagcagcgcacgcgtgaatgcaacgggccttcctacgggggtgcggagtgccagggccactgggtggagacccgagactgcttcctgcagcagtgcccagtggatggcaagtggcaggcctgggcgtcatggggcagttgcagcgtcacgtgtggggctggcagccagcgacgggagcgtgtctgctctgggcccttcttcgggggagcagcctgccagggcccccaggatgagtaccggcagtgcggcacccagcggtgtcccgagccccatgagatctgtgatgaggacaactttggtgctgtgatctggaaggagaccccagcgggagaggtggctgctgtccggtgtccccgcaacgccacaggactcatcctgcgacggtgtgagctggacgaggaaggcatcgcctactgggagccccccacctacatccgctgtgtttccattgactacagaaacatccagatgatgacccgggagcacctggccaaggctcagcgagggctgcctggggagggggtctcggaggtcatccagacactggtggagatctctcaggacgggaccagctacagtggggacctgctgtccaccatcgatgtcctgaggaacatgacagagattttccggagagcgtactacagccccacccctggggacgtacagaactttgtccagatccttagcaacctgttggcagaggagaatcgggacaagtgggaggaggcccagctggcgggccccaacgccaaggagctgttccggctggtggaggactttgtggacgtcatcggcttccgcatgaaggacctgagggatgcataccaggtgacagacaacctggttctcagcatccataagctcccagccagcggagccactgacatcagcttccccatgaagggctggcgggccacgggtgactgggccaaggtgccagaggacagggtcactgtgtccaagagtgtcttctccacggggctgacagaggccgatgaagcatccgtgtttgtggtgggcaccgtgctctacaggaacctgggcagcttcctggccctgcagaggaacacgaccgtcctgaattctaaggtgatctccgtgactgtgaaacccccgcctcgctccctgcgcacacccttggagatcgagtttgcccacatgtataatggcaccaccaaccagacctgtatcctgtgggatgagacggatgtaccctcctcctccgcccccccgcagctcgggccctggtcgtggcgcggctgccgcacggtgcccctcgacgccctccggacgcgctgcctctgtgaccggctctccaccttcgccatcttagcccagctcagcgccgacgcgaacatggagaaggcgactctgccgtcggtgacgctcatcgtgggctgtggcgtgtcctctctcaccctgctcatgctggtcatcatctacgtgtccgtgtggaggtacattcgctcagagcgttctgtcatcctcatcaacttctgcctgtccatcatctcctccaatgccctcatcctcatcgggcagacccagacccgcaacaaggtgatgtgcacgctggtggccgccttcctgcacttcttcttcctgtcctccttctgctgggtgctcaccgaggcctggcagtcctacatggccgtgacgggccacctccggaaccgcctcatccgcaagcgcttcctctgcctgggctgggggctccctgcactggttgtggccatttctgtgggattcaccaaggccaaagggtacagcaccatgaactactgctggctctccctggaggggggactgctctatgccttcgtgggacctgccgctgccgttgtgctggtgaacatggtcattgggatcctggtgttcaacaagctcgtgtccaaagacggcatcacggacaagaagctgaaggagcgggcaggggcctccctgtggagctcctgcgtggtgctgccgctgctggcgctgacctggatgtcggctgtgctcgccgtcaccgaccgccgctccgccctcttccagatcctcttcgctgtcttcgactcgctggagggcttcgtcatcgtcatggtgcactgtatcctccgtagagaggtccaggacgctgtgaaatgccgtgtggttgaccggcaggaggagggcaacggggactcagggggctccttccagaacggccacgcccagctcatgaccgacttcgagaaggacgtggatctggcctgtagatcagtgctgaacaaggacatcgcggcctgccgcactgccaccatcacgggcacactgaagcggccgtctctgcccgaggaggagaagctgaagctggcccatgccaaggggccgcccaccaatttcaacagcctgccggccaacgtgtccaagctgcacctgcacggctcaccccgctatcccggcgggcccctgcccgacttccccaaccactcactgaccctcaagagggacaaggcgcccaagtcctccttcgtcggtgacggggacatcttcaagaagctggactcggagctgagccgggcccaggagaaggctctggacacgagctacgtgatcctgcccacggccacggccacgctgcggcccaagcccaaggaggagcccaagtacagcatccacattgaccagatgccgcagacccgcctcatccacctcagcacggcccccgaggccagcctccccgcccgcagcccgccctcccgccagccccccagcggcgggccccccgaggcaccccctgcccagcccccaccgcctccgcccccaccgccaccacctccccagcagcccctgcccccaccgcccaatctggagccggcaccccccagcctgggggatcccggggagcctgccgcccatccgggacccagcacggggcccagcaccaagaacgagaatgtcgccaccttgtctgtgagctccctggagcggcggaagtcgcggtatgcagaactggactttgagaagatcatgcacacccggaagcggcaccaagacatgttccaggacctgaaccggaagctgcagcacgcagcggagaaggacaaggaggtgctggggccggacagcaagccggaaaagcagcagacgcccaacaagaggccctgggagagcctccggaaagcccacgggacgcccacgtgggtgaagaaggagctggagccgctgcagccgtcgccgctggagcttcgcagcgtggagtgggagaggtcgggcgccacgatcccgctggtgggccaggacatcatcgacctccagaccgaggtctgagcgggtgggcggcggccacgcactgggccacggaggagggatgctgctccgcccgctcctgccgcagacgggcacagacacgctcgcgggcagcgggccaggcccgcaccccggcctcagggcgctcagacggcggccaggcacagggcccgcagtgctgggaccagagccagatgcaggacaggaggcggcccggccagcgggcacagggcaccagaggccgaaggtgcctcagactccgccctcctcgggccgaggcccagcgggcagatgggcggacggctgtggaccgtggacaggcccagcgcggccagcgtcccagggtacccgcctgagctcctgctgcggaggagctgcctgcttggcccggccggcctggcaccgttttttaaacacccccatccctcgggaagcagccagctccccacaccttccagggccctaggcccctcctagacccaggtggagggcacagccctccgaccctcatggcccccaggggcaggactgagtcccctccaggaagaagcaggggggaatctattttttctctccttttcttttcttcaataaaaagaattaaaaacccaaaaaaaa

SEQ ID NO: 68 - DNA, 398 bp, Unknown
  Description of Unknown Organism:Unknown
cggggcaacccgctggagtggacgggccaggtgacggtgcgcaagaagcgcaagccctactccaagttccagacgctcgagctcgagaaggagttcctcttcaacgcgtacgtcagcaagcagaagcgctgggagctggcgcgcaacctcaacctcaccgagcgccaggtcaagatctggttccagaaccggcgcatgaagaacaagaagaacagccagcgccaggcggccagcagcagcagcagcaacagcagcagcagcagcagcagcaacagcagcaagcggccgccggcggggcgtcggccgccgccaacggccaccagggccaccaagcgcaccaccacgcgccccccaacggcgccgtcgcagccctcaagcaccaccagtgacccgtagcg

SEQ ID NO: 69 - DNA, 8670 bp, Unknown
  Description of Unknown Organism:Unknown
cccgggtgcggtgtcgtgtgtggggctgggcgccatgttcctggacatgctgagggccaagcgcgacacggcgcccgaccgccgccagctggacgaccggatgatgggggcggacccgggggacatagcggccaaggtgagggcagggttttgcgtgcgtgcttgattgtgcgtgtgcgtgcgtgcgtgcgtgcgtgcggtgttgcgtgtgtatttgaactgtgttttgtgtatgtacttaggggtaagagtgcatacacatgcatgcgaccggtggccttacaaatcaacaacacgtacgcctgcatgtatccaggtggcagcgtggcgacgagcacgtggcttcgagggcccaggcacggcgggccccagcggcagcgccgccagtggcagcggcgccagcggctcggcaccgcaggcgcgctcgccccgacctcagccaccgcggccgcgctcaccttcacgcgggtgaaccccggcgaggagccgcccgtgtacgcgtgcgagcaaacaggtgcgtaagcgacgtgtgggcagcgcgaagaggcgtgggggcgagagagcaaagggactagggaaacgcacagccaaatacggtatgcgggcaacgaggcgatggccctggaaatcgcagggcccttttgaaatcgtgtaaggcgcaattgctgggcgactaccgtagtctactgatgcattgcactacttgtattactgtatcctactgcagtagtgccgttgccagccgcgctgctgccctttggctcccttcccaatccaaatggcccatgcctcgcgcactccgagcacccagagcacccagaagccgttgcgtgcgctccgccgccgccctctcccccgccttcacttcttaattaatcgtgaatgtaatcccccccccccccgcttcctcaggctgggtgcacgtgtgcgcgacgcctgcacggagggtgtggtggatgcccgcagcgaactgctggtgtgcccggtgagtcgacgaggaggaggtgcaagggggataccagcgcgtgtttctcagggcctgtgtgggacaccgaaacgtggtaaaagagacccgcccgcgaactgtgtatgtggagtagcgtggcgtgtgcggccggaccgacaaggcagcttgtggactgccccacgttgcagagtcagctgacaacgacacgtgcgccttcctgtcattgcccgtgcgcacgcacgtcctccgcactcccaacaaattgacagcgacacgtgcgccttcctataagcctatgcccgcacacgctcccgcgccctcaggtgtcgggccagaccacagaccggttggtccacgagtgcgaggaggatgaggcgggcggctgcggcggcgccggcggggcgccgcggcgaggaggacggcctgggactgggcatcacaggtgggtggcaggctggcagggactcacgcatgggccttgtacgtgactgcggttctgcatggctagtggctcacgcgctgcgcacgttcacgtacggcttgtgggcatgcagtgccttgacgtgaggctgcgctgccttgctgctgccgccttgccccgctccctgcacacactgcagccggcttcgggcgctacttcaccgcgggctacgagtgcgagaacgcgcagcagctcaacaggctgctggggtacaaggcgctgtgagagcgcgccgcagggggagtgtgttcatattgtggttgtttgggccgtgggcgcgggctgcatgtgcgtattgcacgcgtacagcattggtgactggtcaggtgtaagcggccggcagtgcgccgcgaggcgctgcagcgagttgtggggcatgcgtcatgcgcagacggcccctggacgacaaggcgttgagttggcgtttggaggtgtgggacgacgtggggtttgtgccgtcaaagcacagaacagaaggcgtgaccgttttacgagctcgtatgatgtagcatggattgaataatgacatgtgatttttgttacaagcgacgaatgcgtggggttttggatggcaggggtttcagtcgcccgattgcgcatgcacacgtgaccaaatttatgctcaacgacgtgaccattgctttatacatacttgtgtatcggttggcacttataacaattggctcgtcaaattgacgcgaggctgcacttcgatcctgaaagccccagttcaacaagtcggatagccaaatggccccgctcgctctccagcatcaaggggcctctaagtgcctcgcggcaacccagcgcaagtgtgctcgcgttgcggtgagctggactcgtgcacttgtcgacgccgtcggcaccgcaatcgaaagacgcgtgcgtcgagcaattgtggaagccgctgacgaattgtccgcatgtgacattgcaggctcgcgtccccgctcgtctcagcgtcatggcccaggtgcggacgttgggactgcacttgcacgaatgtgatggggccgcaccgagtctgcgcggacgtctcgctgacgtttcgcgttgaatgcatctcgcaataggcagctgctgcgcctgctgacaacactaagaagctgtggggcggtcgcttcacgggcaagacggacccgctcatggagaagttcaacgagtcgctgccctttgacaagcgcctgtgggctgaggacatcaaggtgcggcacagggaggggggcgagtggtggggtggggctgggggggacgcgggtttggtggccagggcagggagggaagacgtgcggggctaggcaagaggctgcgagggcccagggtaacaccagaccgtgccgtgtcgcgtgcccggcttgctgcccaccttgcccggccatccccaccgccctccccaccagcaatgacacgtacacattcacacactcccccacacccacatacccacacacccacgcattccccaacagggcagccaggcgtacgccaaggctcttgccaaggccggcattctggcacatgacgaggccgtgaccattgtggaggggctggccaaggtgcgcacacccggcagcagggcgggtgggtgggtgggtggggtgggggggcagagagaggcgcgggctgagagggggctgagaggggggtcagcgaggcgcaggctcagggggaggcgtctgaggggggctgagatggtggtgggggagctgcgggtgctggggctgctgcggtggcgggcgggcgggcgggcgggcgacgtgtacgtgagtagccgctgaccgggcgctgggcctttgcgcacgccacagcccacatgacaccgccgcaaggcccgccgcgccccacccacgttcacacactccccacacccacgcgtgcgcgcgcctccttcccctcaatacacgcgcctccttcccctggcccccgcctgctccccccatccggccgccccgcctgcaggtggctgaggagtggaaggcgggtgcctttgtgatcaaggcgggtgacgaggacatccacacggccaacgagcggcgcctcacggagctggtgggggcggtgggcggcaagctgcacaccggccgctcgcgcaacgaccaggtgagggtgggtgggtgggggtggggtgggtgggtgggtgggtgggtgggtgggtgggtgggtgggtgggtgggtgggtgggggtttgagataccggtaccaggccaaactaaaccgaacccaagggggtggcgtaggggcgtgggagggggggagtgcggaagccgggaggcaggagtaagggcgggaggagggggccggaggagaagcagggacgaagtcgatgacaggcgcagtcggtggcggcggtggcgggtgtgccgttgtgcagtggctgtggaggccatgtgcagggcggcggcggggccgggccgggggtgggagacttgtccagaccccgtggccctcttccagccccgtccgccactgccgccaccaccaccgccgccgccgtagccaccacccctcacgtcgaggcacttcacagatgcgaagcaaccacaccgttctccacatgaacagctaccctcccaaacccaactttcccttcccgccttacctaaccatgacccgctaccccccccccctttatttcttaactaaccatgaatgcccccccccggctgtacctggctacgacttcacttcgtaaacttaatgtgtgtaaccccccttacacacacacacacacccctccccgcccctccaaaggttgccaccgactaccggctgtggctggtgggtcaggtggaggtgatgcggtccgaggtgggcgagctgatgcgcgtggcggcggaccgctccgaggcagaggtggaggtgctcatgccgggtgagggggcagggagggggggagggggagggggaggtgctcatgccggtgagggtagggaggggaggggcagaggagggagggggaggagggggcggctgagtgcgggagaggcagggatgagggcgatagaaagttgcgtattgtcggtaaactcaaaggactagacgaagagaacaaacctaaacaagggagctggagcgaggccaaatctgaacgtgacatcgcccgcctcctcccgctgcctgctcccccacctcctcccccatctcgcccccccccccacacacacacaggcttcacgcaccttcagaatgccatgactgtgcgctggagccactggctgatgagccacgccgcggcctggcagcgcgacgacatgcggctgcgggacctgctgccgcgggtggccacactgccgctgggctcgggtgggtgagggaggggaggggaggggagggggggagggggagggagaggaggggagaagggggggggagacgaggagggtggaagggtgggggcggggcggtggaggctagagggtggggctgggtgggtggacggagtgcactggtagaggagggatagggtacattgagacgggaggagggatgcaggggcgaaggtggggaggaggggaggggaggaggcgtggagctggagtgggccgacgagtgtgcggacggggcaggcggcaacggggattaaacggcggggggccggggcgtgtgcacgacaggggcttgcgcgtctgcgattgtgggggcacacagggacaggagcacgacgtgggacacgcatagatacgccgcattgacaacacacacacacacacacacacacacacacacacacacacacacacaaacacaaacacacacaaacacaaacacacacacgcccccccccctacacacacgccccctccccaggcgccctggccggcaacccctttctggtggaccgccagttcatcgccaaggagttgggtttcggcggcggcgtgtgccccaactccatggacgcggtgaggggaggaggagggggaggagggcgggggggggcaggaggggggaggaggagggggggagggggttaactttgaagcgtaaggaaacagtcgggaggagggggggaaggagggggcctggaggagggggggaggaggagggtggctggagggggctgggggaggaggagggggaggattgggagggggctgggggagggtgcccgcagctgggggaggtggggagggagggggttgctgctggtgtaaagggcctgtaggcactgagagcactgtggggagccggggtactgcctggggccccgcgctgcagaggtgtcgcgcagtgtggcggcgcatcccccgcatccccacacgcgggccgctgccgctgcccgccacacccttgccactttgtgtgctttcctaggatatacacacacacacacacacacacacacacacacacacacacaaacacaaacacacacgggcgcgggctttcgtttcgttttttaacacaaacacacactccccctgtgctcctcaacacactccatctttctcacacaaacacacacgcacacacacatgcgcaggtgtctgaccgcgactttgtgatcgagacggtgtttgcggccagcctgctgtgcgtgcacctgtcgcgctgggcggaggacctcatcatctacagctccggccccttcggctacgtgcagtgcagcgacgcctacgccaccggctcctcgctcatgccgcagaagaagaaccccgacgccctggagctcatcaggtgcgggagggatggggtgggggtgggggggttacattcatggttagttaagaagtgaaggcgtagggggtggatggggtgggttacattcatgaacatttaagaagtgaaggcgtagccaggaacagtagtagagcagacgcgttgtagtgtgtgggtttgggtgggagggatggttgggtaaagcggtacaggatgtactgaggactgcagaccgaaggagcgggggagggggagcaggcaggcggggcgaggggcgtgggggcgggggttactggcaccgtgccgggtaagcaacacgtgacacggagatgcaccacacaaagagggacgtggggagtggcaggcgggggccagggctgagaggcgcgtgtggaggggtgcggggttgggcggggggctgtttcatgataccgctgcctccacctcctccaccgcctcctgccacctccacctcccccactgcccctccccgcctcctcctgctgcaggggcaagggcggtcgtgtgcagggcaacctgatgggcgtcatggcggtgctcaagggcacgcccaccacatacaacaaggacttccaggcgagagagcgagagcgagggagggagggagagcgagggagagggagggagagggagggagagggagacagagggacagggacagggacagggacagggacagggacagggacaggggcaggggcaggggcaggggcaggggcaggggcaggggaggccccccgggggcggcgggcccggggcatgaggtcagacataggggcgctgcactgaggccgcgaggcgggcgggaggcagggggcggggggcggggggcgggagcggacatgcgccgcaaacacagacgggttgagaaagcacaacgactggaacgcagtgggcttactgacaattcatcattgtgcgcatatgtgtgtatgtgtatgtgtgtgtttgtttgtgcaggagtgttgggagctgctgtttgacacggtggacacggtgcacgacgtggtgcgcatcgccaccggcgtgctgtccaccctgcggatcaagcccgaccgcatgaaggccggtgagcgtagccgagcagggctggagcagcagccgggcagcagtagcagcagggcaggggagcagcgggagcgggagcagcaggaggggtggttgggaagcggtgggggtagggtgggagcggaggaagggaaggaggagcaggagcaggaggaagaggaggaggaagggcggtggggggtggggggtcgtgtccttggccgcatgggcggaggcggggaggcggggaggaggcggggaagcagagcctgcacccacgctccgcgggtccctaccgtcttgcgcctaaccccgtgcgcctagcctcttgcgcccacccccttagtgcatcctgtacccctctttccaaacatccttgcaactccctgacctcctcgccaaacctcccccgcccccaggcctgtccgccgacatgctcgccacggacttggccgagtacctggtgcgcaagggcgtgccgttccgggagacacaccaccacaggtgcggccgggcgggagggcgtgagggcgtgggtggggcatgcccggggttgtgagagctatcgaacgttgtgccgcgcctgtttcacaatgtcgggccacagggtatgcagtttcctctccatatgtataacaaactgaccaccaatcatgcacgctcacacgctctcccacacacacgcgcaccacgccaccacagcggcgccgccgtgaagatggccgaggaccgcggctgcacgctgttcgacctcaccgtggacgacctcaagaccatccacccgctcttcaccgacgacgtggcggcggtgagcggcggcgcggagcagcagcagcagcagcagcagcagcagcagcagcagcagtagcctgggggggagcgtgtgggaggaacggcgggggaggggaggcggggggtgtcgtttgcagccgagcgcacgtggtgctttgccccattccatgccagcagggtgacacacctgaccatgctggtgtgctgctaggtggttcacacctacgtgtgaatttgtgctggcgtgcgcacaccttactgtggccatgtgaacggcatcctcatgtcctcgtgattgcgcccggcacattgcccacaaccccgcaccacccagctcctcaatccagtgcaaggaaaggaaatgcacgcccgccgcaccaacaacacgacgcatgtgtttgccacgtgcgcgcacacacgcgcaggtgtgggacttcaaccgcagcgccgagatgcgcgacacggagggcggcaccagcaagcgctcggtgctggagcaggtgcagaagatgcgcacctacctggcggcggagggacagcactgagcgggtcgggggagggggggcgggtgtgtatgtgtgtgtgtgtgcgtgtgtaagtctcggtggaggggtggtcctctatatggcggcggggccacagggggacgggtgtgacagagttacggccggagccagcggagtcccgggatggattaaggatc

SEQ ID NO: 70 - DNA, 745 bp, Unknown
  Description of Unknown Organism:Unknown
atgagatggcgacgcgccccgcgccgctccgggcgtcccggcccccgggcccagcgccccggctccgccgcccgctcgtcgccgccgctgccgctgctgccactactgctgctgctggggaccgcggccctggcgccgggggcggcggccggcaacgaggcggctcccgcgggggcctcggtgtgctactcgtccccgcccagcgtgggatcggtgcaggagctagctcagcgcgccgcggtggtgatcgagggaaaggtgcacccgcagcggcggcagcagggggcactcgacaggaaggcggcggcggcggcgggcgaggcaggggcgtggggcggcgatcgcgagccgccagccgcgggcccacgggcgctggggccgcccgccgaggagccgctgctcgccgccaacgggaccgtgccctcttggcccaccgccccggtgcccagcgccggcgagcccggggaggaggcgccctatctggtgaaggtgcaccaggtgtgggcggtgaaagccgggggcttgaagaaggactcgctgctcaccgtgcgcctggggacctggggccaccccgccttcccctcctgcgggaggctcaaggaggacagcaggtacatcttcttcatggagcccgacgccaacagcaccagccgcgcgccggccgccttccgagcctctttcccccctctggagacgggccggaacctcaagaaggaggtcagccgggtgctgtgcaagcggtgcg

SEQ ID NO: 71 - DNA, 1986 bp, Unknown
  Description of Unknown Organism:Unknown
gaattcctttttttttttttttttttctttttttttttgcccttatacctcttcgcctttctgtggttccatccacttcttccccctcctcctcccataaacaactctcctacccctgcacccccaataaataaataaaaggaggagggcaaggggggaggaggaggagtggtgctgcgaggggaaggaaaagggaggcagcgcgagaagagccgggcagagtccgaaccgacagccagaagcccgcacgcacctcgcaccatgagatggcgacgcgccccgcgccgctccgggcgtcccggcccccgggcccagcgccccggctccgccgcccgctcgtcgccgccgctgccgctgctgccactactgctgctgctggggaccgcggccctggcgccgggggcggcggccggcaacgaggcggctcccgcgggggcctcggtgtgctactcgtccccgcccagcgtgggatcggtgcaggagctagctcagcgcgccgcggtggtgatcgagggaaaggtgcacccgcagcggcggcagcagggggcactcgacaggaaggcggcggcggcggcgggcgaggcaggggcgtggggcggcgatcgcgagccgccagccgcgggcccacgggcgctggggccgcccgccgaggagccgctgctcgccgccaacgggaccgtgccctcttggcccaccgccccggtgcccagcgccggcgagcccggggaggaggcgccctatctggtgaaggtgcaccaggtgtgggcggtgaaagccgggggcttgaagaaggactcgctgctcaccgtgcgcctggggacctggggccaccccgccttcccctcctgcgggaggctcaaggaggacagcaggtacatcttcttcatggagcccgacgccaacagcaccagccgcgcgccggccgccttccgagcctctttcccccctctggagacgggccggaacctcaagaaggaggtcagccgggtgctgtgcaagcggtgcgccttgcctccccaattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacagtacgtccactccctttctgtctctgcctgaataggagcatgctcagttggtgctgctttcttgttgctgcatctcccctcagattccacctagagctagatgtgtcttaccagatctaatattgactgcctctgcctgtcgcatgagaacattaacaaaagcaattgtattacttcctctgttcgcgactagttggctctgagatactaataggtgtgtgaggctccggatgtttctggaattgatattgaatgatgtgatacaaattgatagtcaatatcaagcagtgaaatatgataataaaggcatttcaaagtctcacttttattgataaaataaaaatcattctactgaacagtccatcttctttatacaatgaccacatcctgaaaagggtgttgctaagctgtaaccgatatgcacttgaaatgatggtaagttaattttgattcagaatgtgttatttgtcacaaataaacataataaaagg

SEQ ID NO: 72 - DNA, 2003 bp, Unknown
  Description of Unknown Organism:Unknown
  Misc_feature (31): N is any nucleic acid
  Misc_feature (32): N is any nucleic acid
ggaattcctttttttttttttttttttcttnntttttttttgcccttatacctcttcgcctttctgtggttccatccacttcttccccctcctcctcccataaacaactctcctacccctgcacccccaataaataaataaaaggaggagggcaaggggggaggaggaggagtggtgctgcgaggggaaggaaaagggaggcagcgcgagaagagccgggcagagtccgaaccgacagccagaagcccgcacgcacctcgcaccatgagatggcgacgcgccccgcgccgctccgggcgtcccggcccccgggcccagcgccccggctccgccgcccgctcgtcgccgccgctgccgctgctgccactactgctgctgctggggaccgcggccctggcgccgggggcggcggccggcaacgaggcggctcccgcgggggcctcggtgtgctactcgtccccgcccagcgtgggatcggtgcaggagctagctcagcgcgccgcggtggtgatcgagggaaaggtgcacccgcagcggcggcagcagggggcactcgacaggaaggcggcggcggcggcgggcgaggcaggggcgtggggcggcgatcgcgagccgccagccgcgggcccacgggcgctggggccgcccgccgaggagccgctgctcgccgccaacgggaccgtgccctcttggcccaccgccccggtgcccagcgccggcgagcccggggaggaggcgccctatctggtgaaggtgcaccaggtgtgggcggtgaaagccgggggcttgaagaaggactcgctgctcaccgtgcgcctggggacctggggccaccccgccttcccctcctgcgggaggctcaaggaggacagcaggtacatcttcttcatggagcccgacgccaacagcaccagccgcgcgccggccgccttccgagcctctttcccccctctggagacgggccggaacctcaagaaggaggtcagccgggtgctgtgcaagcggtgcgccttgcctccccaattgaaagagatgaaaagccaggaatcggctgcaggttccaaactagtccttcggtgtgaaaccagttctgaatactcctctctcagattcaagtggttcaagaatgggaatgaattgaatcgaaaaaacaaaccacaaaatatcaagatacaaaaaaagccagggaagtcagaacttcgcattaacaaagcatcactggctgattctggagagtatatgtgcaaagtgatcagcaaattaggaaatgacagtgcctctgccaatatcaccatcgtggaatcaaacgctacatctacatccaccactgggacaagccatcttgtaaaatgtgcggagaaggagaaaactttctgtgtgaatggaggggagtgcttcatggtgaaagacctttcaaacccctcgagatacttgtgcaagtgcccaaatgagtttactggtgatcgctgccaaaactacgtaatggccagcttctacagtacgtccactccctttctgtctctgcctgaataggagcatgctcagttggtgctgctttcttgttgctgcatctcccctcagattccacctagagctagatgtgtcttaccagatctaatattgactgcctctgcctgtcgcatgagaacattaacaaaagcaattgtattacttcctctgttcgcgactagttggctctgagatactaataggtgtgtgaggctccggatgtttctggaattgatattgaatgatgtgatacaaattgatagtcaatatcaagcagtgaaatatgataataaaggcatttcaaagtctcacttttattgataaaataaaaatcattctactgaacagtccatcttctttatacaatgaccacatcctgaaaagggtgttgctaagctgtaaccgatatgcacttgaaatgatggtaagttaattttgattcagaatgtgttatttgtcacaaataaacataataaaaggaaaaaaaaaaaaaa

SEQ ID NO: 73 - DNA, 957 bp, Unknown
  Description of Unknown Organism:Unknown
  Misc_feature (809): N is any nucleic acid
  Misc_feature (810): N is any nucleic acid
  Misc_feature (811): N is any nucleic acid
tctcgccccaactttttcccccgcgctccgcagcagcagcagcagcagcagcagcagcagcaaaatggcagacctcttcagcggactcgtgggcggcgtcgtcggcgctgttgctgcagcagatttgcctgcggagggcgagagggccccccgccccgcccccggcactgcctggacttgctgctgcagcaaactgcaagaaggggcccgcgagctggagggttttgtgcagcagctgagttttgttgcagggaagctggcctgctgcctgcgggtgggggcggagcagctggcgcgctgcgctgcggaggggcggctgcccagcagcagcagcagcagcagctgctgcgcgctgctgcagctcgagaagcaggacctcgagcagagcctcgaggccggcaagcagggcgcggagtgcctcttgaggagcagcaaactggccctcgaggccctcctcgagggggcccgcgttgcagcaacgcggggtttgctgctggtcgagagcagcaaagacacggtgctgcgcagcattccccacacccaggagaagctggcccaggcctacagttctttcctgcggggctaccagggggcagcagcggggaggtctctgggctacggggcccctgctgctgcttacggccagcagcagcagcccagcagctacggggcgccccccgcctccagccagcagccctccggcttcttctggtagccctgcagcagcagcagcagcagcagcagcagcagcagcgcgggcggcagccgcggcggggccggggcgccgctgcagcaacagcagcagccgnnncggctagcgccgcggagcactcgcagggaactccacaggcagcgggagagcagcagggacgagaagcaggtcatgtagcgcaggcagcagcgccagctgcagcagcagcagcagcagcagcagcagcagcagcagctcctgcaccg

SEQ ID NO: 74 - DNA, 957 bp, Unknown
  Description of Unknown Organism:Unknown
  Misc_feature (809): N is any nucleic acid
  Misc_feature (810): N is any nucleic acid
  Misc_feature (811): N is any nucleic acid
tctcgccccaactttttcccccgcgctccgcagcagcagcagcagcagcagcagcagcagcaaaatggcagacctcttcagcggactcgtgggcggcgtcgtcggcgctgttgctgcagcagatttgcctgcggagggcgagagggccccccgccccgcccccggcactgcctggacttgctgctgcagcaaactgcaagaaggggcccgcgagctggagggttttgtgcagcagctgagttttgttgcagggaagctggcctgctgcctgcgggtgggggcggagcagctggcgcgctgcgctgcggaggggcggctgcccagcagcagcagcagcagcagctgctgcgcgctgctgcagctcgagaagcaggacctcgagcagagcctcgaggccggcaagcagggcgcggagtgcctcttgaggagcagcaaactggccctcgaggccctcctcgagggggcccgcgttgcagcaacgcggggtttgctgctggtcgagagcagcaaagacacggtgctgcgcagcattccccacacccaggagaagctggcccaggcctacagttctttcctccggggctaccagggggcagcagcggggaggtctctgggctacggggcccctgctgctgcttacggccagcagcagcagcccagcagctacggggcgccccccgcctccagccagcagccctccggcttcttctggtagccctgcagcagcagcagcagcagcagcagcagcagcagcgcgggcggcagccgcggcggggccggggcgccgctgcagcaacagcagcagccgnnncggctagcgccgcggagcactcgcagggaactccacaggcagcgggagagcagcagggacgagaagcaggtcatgtagcgcaggcagcagcgccagctgcagcagcagcagcagcagcagcagcagcagcagcagctcctgcaccg

SEQ ID NO: 75 - DNA, 1089 bp, Unknown
  Description of Unknown Organism:Unknown
  Misc_feature (376): N is any nucleic acid
  Misc_feature (377): N is any nucleic acid
  Misc_feature (847): N is any nucleic acid
  Misc_feature (848): N is any nucleic acid
  Misc_feature (849): N is any nucleic acid
  Misc_feature (850): N is any nucleic acid
gaattccctccaactcttcgcgactctctctctctcgccccaactttttcccccgcgccccgcagcagcagcagcagcagcagcagcaaaatggcagacctcttcagcggactcgtgggcggcgtcgtcggcgctgttgctgcagcagatttgcctgcggagggcgagagggccccccgccccgcccccggcactgcctggacttgctgctgcagcaaactgcaagaaggggcccgcgagctggagggttttctgcagcagctgagttttgttgcagggaagctggcctgctgcctgcgggtgggggcggagcagctggcgcgctgcgctgcggaggggcggctgcccagcagcagcagcagcagcagctgctgcnngctgctgcagctcgagaagcaggacctcgagcagagcctcgaggccggcaagcagggcgcggagtgcctcttgaggagcagcaaactggccctcgaggccctcctcgagggggcccgcgttgcagcaacgcggggtttgctgctggtcgagagcagcaaagacacggtgctgcgcagcattccccacacccaggagaagctggctcaggcctacagttctttcctgcggggctaccagggggcagcagcggggaggtctctgggctacggggcccctgctgctgcttacggccagcagcagcagcccagcagctacggggcgccccccgcctccagccagcagccctccggcttcttctggtagccctgcagcagcagcagcagcagcagcagcagcagcagcggcggcggcagccgcggcggggccggggcgccgctgcagcaacagcagcagccgcggcggctagcgnnnngagcactcgcagggaactccacaggcagcgggagagcagcagggacgagaagcaggtctatgtagcgcaggcagcagcgccagctgcagcagcagcagcagcagcagcagcagcagcagcagctcctgcaccgcagcgttgtgtcatttattacgttggcagctctgaggcctcggcgcagccaacgcgcctcaggtatctttcagactcttttctctaaggtcttccagacggaattc

SEQ ID NO: 76 - DNA, 1985 bp, Unknown
  Description of Unknown Organism:Unknown
cgccgagctttcggcacctctgccgggtggtaccgagccttcccggcgccccctcctctcctcccaccggcctgcccttccccgcgggactatcgcccccacgtttccctcagcccttttctctcccggccgagccgcggcggcagcagcagcagcagcagcagcaggaggaggagcccggtggcggcggtggccggggagcccatggcgtacagtcaaggaggcggcaaaaaaaaagtctgctactactacgacggtgatattggaaattattattatggacagggtcatcccatgaagcctcatagaatccgcatgacccataacttgctgttaaattatggcttatacagaaaaatggaaatatataggccccataaagccactgccgaagaaatgacaaaatatcacagtgatgagtatatcaaatttctacggtcaataagaccagataacatgtctgagtatagtaagcagatgcatatatttaatgttggagaagattgtccagcgtttgatggactctttgagttttgtcagctctcaactggcggttcagttgctggagctgtgaagttaaaccgacaacagactgatatggctgttaattgggctggaggattacatcatgctaagaaatacgaagcatcaggattctgttacgttaatgatattgtgcttgccatccttgaattactaaagtatcatcagagagtcttatatattgatatagatattcatcatggtgatggtgttgaagaagctttttatacaacagatcgtgtaatgacggtatcattccataaatatggggaatactttcctggcacaggagacttgagggatattggtgctggaaaaggcaaatactatgctgtcaattttccaatgtgtgatggtatagatgatgagtcatatgggcagatatttaagcctattatctcaaaggtgatggagatgtatcaacctagtgctgtggtattacagtgtggtgcagactcattatctggtgatagactgggttgtttcaatctaacagtcaaaggtcatgctaaatgtgtagaagttgtaaaaacttttaacttaccattactgatgcttggaggaggtggctacacaatccgtaatgttgctcgatgttggacatatgagactgcagttgcccttgattgtgagattcccaatgagttgccatataatgattactttgagtattttggaccagacttcaaactgcatattagtccttcaaacatgacaaaccagaacactccagaatatatggaaaagataaaacagcgtttgtttgaaaatttgcgcatgttacctcatgcacctggtgtccagatgcaagctattccagaagatgctgttcatgaagacagtggagatgaagatggagaagatccagacaagagaatttctattcgagcatcagacaagcggatagcttgtgatgaagaattctcagattctgaggatgaaggagaaggaggtcgaagaaatgtggctgatcataagaaaggagcaaagaaagctagaattgaagaagataagaaagaaacagaggacaaaaaaacagacgttaaggaagaagataaatccaaggacaacagtggtgaaaaaacagataccaaaggaaccaaatcagaacagctcagcaacccctgaatttgacagtctcaccaatttcagaaaatcattaaaaagaaaatattgaaaggaaaatgttttctttttgaagacttctggcttcattttatactactttggcatggactgtatttattttcaaatgggactttttcgtttttgtttttctgggcaagttttattgtgagattttctaattatgaagcaaaatttcttttctccaccatgctttatgtgatagtatttaaaattgatgtgagttattatgtcaaaaaaactgatctattaaagaagtaattggcctttctgagctgaaaaaaaaaaaaaaaaag

SEQ ID NO: 77 - DNA, 476 bp, Unknown
  Description of Unknown Organism:Unknown
ccaccctcctccccctcccccggccacttcgctaacttggtggctgttgtgatgcgtattcctgtagatccgagcaccagccggcgcttcagccccccctccagcagcctgcagcccggcaaaatgagcgacgtgagcccggtggtggctgcgcaacagcagcagcaacagcagcagcagcaacagcagcagcagcagcagcaacagcagcagcagcagcaggaggcggcggcggcggctgcggcggcagcggcggctgcggcggcggcagctgcagtgccccggttgcggccgccccacgacaaccgcaccatggtggagatcatcgccgaccacccggccgaactcgtccgcaccgacagccccaacttcctgtgctcggtgctgccctcgcactggcgctgcaacaagaccctgcccgtggccttcaaggtaagaggctaccccgccccccgcccccggccgggagcggcgga

SEQ ID NO: 78 - DNA, 24 bp, Artificial Sequence
  Description of Artificial Sequence:DNA Primer
gcattttggatccgccttttcatg

SEQ ID NO: 79 - DNA, 22 bp, Artificial Sequence
  Description of Artificial Sequence:DNA Primer
gttgtgtgctgcagattgttcc

SEQ ID NO: 80 - DNA, 21 bp, Artificial Sequence
  Description of Artificial Sequence:DNA Primer
gaaaaatggggatccgaggtg

SEQ ID NO: 81 - DNA, 20 bp, Artificial Sequence
  Description of Artificial Sequence:DNA Primer
gcaggagaattccgtccatg

SEQ ID NO: 82 - protein, 5 aa, Homo sapiens
  Misc_feature (3): Xaa is any amino acid
WSXWS

SEQ ID NO: 83 - protein, 6 aa, Homo sapiens
CSVTCG

SEQ ID NO: 84 - protein, 5 aa, Homo sapiens
  Misc_feature (4): Xaa is any amino acid
GCQXR

SEQ ID NO: 85 - DNA, 733 bp, Homo sapiens
gggatccggagcccaaatcttctgacaaaactcacacatgcccaccgtgcccagcacctgaattcgagggtgcaccgtcagtcttcctcttccccccaaaacccaaggacaccctcatgatctcccggactcctgaggtcacatgcgtggtggtggacgtaagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccaacccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccgcgactctagaggat

SEQ ID NO: 86 - DNA, 86 bp, Artificial Sequence
  Description of Artificial Sequence:DNA Primer
gcgcctcgagatttccccgaaatctagatttccccgaaatgatttccccgaaatgatttccccgaaatatctgccatctcaattag

SEQ ID NO: 87 - DNA, 27 bp, Artificial Sequence
  Description of Artificial Sequence:DNA Primer
gcggcaagctttttgcaaagcctaggc

SEQ ID NO: 88 - DNA, 271 bp, Artificial Sequence
  Description of Artificial Sequence:PCR Fragment
ctcgagatttccccgaaatctagatttccccgaaatgatttccccgaaatgatttccccgaaatatctgccatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctt

SEQ ID NO: 89 - DNA, 32 bp, Homo sapiens
gcgctcgagggatgacagcgatagaaccccgg

SEQ ID NO: 90 - DNA, 31 bp, Homo sapiens
gcgaagcttcgcgactccccggatccgcctc

SEQ ID NO: 91 - DNA, 12 bp, Homo sapiens
ggggactttccc

SEQ ID NO: 92 - DNA, 73 bp, Homo sapiens
gcggcctcgaggggactttcccggggactttccggggactttccgggactttccatcctgccatctcaattag

SEQ ID NO: 93 - DNA, 27 bp, Artificial Sequence
  Description of Artificial Sequence:PCR Fragment
gcggcaagctttttgcaaagcctaggc

SEQ ID NO: 94 - DNA, 652 bp, Homo sapiens
  Misc_feature (524): N is any nucleic acid
ggcataagatcacactttagttcagagacacatttgcataaatacttgaaatggatccacccctgcaggtggcagcctgagaacatggcgctgcagggggaccagggcagcgtctggttcaggtggacgaacagcggtgccatcacgtggtgcttgcccatgggcccgaagagccgtgtgcagggcttggagtcgtcgtggggcatgctgaggacgtgccctagttcatgggccagggtgtgggccgcctggagcccctcatcctcgatcacggagcagcttttgttggggtcacaaatggtcccgatgtctgccacacccagggtgtcacacagcccctcctgcccacagaagttctgtctggtgagcaggatggccgtgtcgtagtgctctgggtggcggtcgctgggctggttgaaacgccgctgccagttgcagaagttacgcagtgtaagccccccattgtcggacacctctgggccccatttttcatcttctacgatcagcacttttaccaccatcangttgatggaattcttgatgctggggtgcttgtagaatcgggcttgccacgaaaattaacctcaggatgtggttctgcaggtcggcccgtaaagggcgccatggacgcatcggccaccaacagcgtttc

SEQ ID NO: 95 - DNA, 716 bp, Homo sapiens
  Misc_feature (578): N is any nucleic acid
  Misc_feature (658): N is any nucleic acid
  Misc_feature (666): N is any nucleic acid
  Misc_feature (678): N is any nucleic acid
taagtttgctagtcctttgcaaacagactgacgctgagtgtcctgtctgagtcaataagtgcacttttaccttttaacctatgccctctacttgaacccgagcaaggtccagtccactggacagttgatgatagggtctgccgccccataccctctcctcttcccccttaggaatttgtgcagtactggaggggttgcggcaatgggaggcctgggtgggccgtgctgccttgatatggccaagggacccagtcaccacagtggagacccttgtctgcacctcagtaccgcatgtccaggagcacaagactggcccctgcccccctgaatcacagggggcacagctggctttcgcagggcttggcatcctcgggtttcagagccttgttgcaggtggcagaggcctggccggaggggtccctgcactctacagttcgcctctgccagccggccccgcaggtgctagagcactcagaccagtcccccagcacccactgtgcgtggagcagcggctggatgatgttggtggttgctctctctttgctgctctgcatgctaaaagtcacgtcattaggaancaaagaaggtgtatttgactttttggggggaagaacctcgcccaggactgtcaggagctgcactgtcagaaggctctgcnaaggcccngaagctctgcangcgctccagggtggcgatggagccgtgtacttcaggat

SEQ ID NO: 96 - DNA, 543 bp, Homo sapiens
ggcataagatcacactttagttcagagacacatttgcataaatacttgaaatggatccacccctgcaggtggcagcctgagaacatggcgctgcagggggaccagggcagcgtctggttcaggtggacgaacagcggtgccatcacgtggtgcttgcccatggcctcgaagagccgtgtgcagggcttggagtcgtcgtggggcatgctgaggacgtgccctagttcatgggccagggtgtgggccgctggagccctcatcctcgatcacggagcagcttttgttggggtcacaaatggtcccgatgtctgccacacccagggtgtcacacagcccctcctgcccacagaagttctgtctggtgagcaggatggccgtgtcgtagtgctctgggtggcggtcgctgggctggttgaaacgccgctgccagttgcagaagttacgcagtgtaaggcccccattgtcggacagctctggggcccatttttcatcttctacgatcagcacttttaaccacatcaggttgatggaattcttgatgcc

SEQ ID NO: 97 - DNA, 377 bp, Mus musculus
gcaaagtgccaccacccttcggatccaaaactagaagcaagaggtttgtgtccgaggctcgcttcgtggaaacacttctggtggctgatgcgtccatggctgccttctatgggaccgacctgcagaaccacatcctcacggtgatgtcaatggcagcccgaatctacaagcacccgagcatcaagaactccgtcaaccttgtggtggtgaaagtgctaatagtggaagaggaaggatggggcccggaggtgtcggacaacggggggctcacactgcgcaacttctgcagctggcaacggcgtttcaacaagcccagtgaccgccacccggagcactatgacactgccatcttgttcaccagacagaacttctgtggg

SEQ ID NO: 98 - DNA, 432 bp, Rattus norvegicus
  Misc_feature (42): N is any nucleic acid
  Misc_feature (214): N is any nucleic acid
ctaaagtacagtggttccatggccaccctggagcggctgcanagcttccaagccctccctgagcctcttacagtacagctcctgactgtgtctggtgaggtcttccctccaaaagtcaaatataccttcttcgtccccaatgacacggacttcaacgtgcagagtagcaaagaaagagcaagcaccaacatcattcagtccttgccctatgcanagtgggtgctgggggactggtctgaatgtccaagcacatgtggaggtggctggcagcggcggactgtggaatgcagggacccctcaggtcaggcctctgacacctgtgatgaggctctgaaacctgaggatgccaagccctgtggaagccagccatgtctcctctgatccccttggtggacatgtctaaggcttatggatttgggctactggcgtttt

SEQ ID NO: 99 - DNA, 354 bp, Mus musculus
caaagtgcaccacccttcggatccaaaactagaagcaagaggtttgtgtccgaggctcgcttcgtggaaacacttctggtggctgatgcgtccatggctgccttctatgggaccgacctgcagaaccacatcctcacggtgatgtcaatggcagccacgaatctacaagcacccgagcatcaggaactccgtcaaccttgtggtggtgaaagtgctaatagtggaagaggaaggatggggcccggagtgtcggacaacggggggctcacactgcgcaacttctgcagctggcaacggcgtttcaacaagcccagtgaccgccacccggagcactatgacactgccatcttgttc

SEQ ID NO: 100 - DNA, 389 bp, Homo sapiens
  Misc_feature (136): N is any nucleic acid
ttgtgcccagaagacactggccctggggcctgggttgagttcaaaaccaaaagaaagaagaaaagtgctgtaaattcgggatttctccaccggatgctcctgcttccgcatgggtgtcacctccatgccgttcctncctctttctagggaaaagcttcagggagcagcagtgtgagaagtataatgcctacaattacactgacatggacgggaatctcctgcagtgggtccccaagtatgctggggtgtccccccgggaccgcctggcaagttgttctgccgagcccgggggaggagcgagttcaaagtgttcgaggccaaggtgagaatcaccctgggggacttcagatccagagatggggggagggaaggtcggcctgttccccaca

SEQ ID NO: 101 - DNA, 305 bp, Homo sapiens; identical to SEQ ID NO: 43
  Misc_feature (128): N is any nucleic acid
  Misc_feature (146): N is any nucleic acid

SEQ ID NO: 102 - DNA, 152 bp, Homo sapiens; identical to SEQ ID NO: 19
  Misc_feature (105): N is any nucleic acid
  Misc_feature (122): N is any nucleic acid
  Misc_feature (135): N is any nucleic acid

SEQ ID NO: 103 - DNA, 632 bp, Homo sapiens
tttaataataataatgcccggggctttattatgctgtatcactgctcagaggttaataatcctcactaactatcctatcaaatttgcaactggcagtttactctgatgattcaactccttttctatctacccccataatcccaccttactgatacacctcactggttactggcaagatacgctggatccctccagccttcttgctttccctgcaccagcccttcctcactttgccttgccctcaaagctaacaccacttaaaccacttaactgcattctgccattgtgcaaaagtctatgaaatgtttaggtttctttaaaggatcacagctctcatgagataacacccctccatcatgggacagacacttcaagcttctttttttgtaacccttcccacaagtcttagaacatgatgaccactcccccagctgccactgggggcagggatggtctgcacaaggtctggtgctggctggcttcacttcctttgcacactcggaagcaggctgtccattaatgtctcggcattctaccagtcttctctgccaacccaattcacatgacttagaacattcgccccactcttcaatgacccatgctgaaaaagtggggatagcattgaaagaatc

SEQ ID NO: 104 - DNA, 519 bp, Homo sapiens
tttttttctaaacttgtaatagatgtaacaaaagaaataataataataatgcccggggctttattatgctatatcactgctcagaggttaataatcctcactaactatcctatcaaatttgcaactggcagtttactctgatgattcaactccttttctatctacccccataatcccaccttactgatacacctcactggttactggcaagatacgctggatccctccagccttcttgctttccctgcaccagcccttcctcactttgccttgccctcaaagctaacaccacttaaaccacttaactgcattctgccattgtgcaaaagtctatgaaatgtttaggtttctttaaaggatcacagctctcatgagataacacccctccatcatgggacagacacttcaagcttctttttttgtaacccttcccacaggtcttagaacatgatgaccactcccccagctgccactgggggcagggatgtctgcacaagggctggtgctggctgcccggac

SEQ ID NO: 105 - DNA, 475 bp, Homo sapiens
gagtcatgatgcgatcacaaccagcttttacacactgtccttgcacacagacagaggtggaatctgggctacatggagtaccatctacaaccttgggctgcaaaacgaagaagtagccaatgcctttggcttggcagatgagcttgcacctgtcctttggtgagacgccagcgtacttgggaatccattccaccgcaggcccactcccaaaggaagcttttgaaaactcgttgtgtgcttcacattgttcctctctaaaggtttttccattattgtctggacagtcctcaaggttacaggatctgtagcgcactcgtttgccttcacagtacttccctccattctttgggactgggttgtcacattccctcatcgtgtactggactcctccaccgcacgttctcgaacagtctccccaaggcccccacattccccagcttccatgaaaaggcgtatcaaaatgctttctgtcggt

SEQ ID NO: 106 - DNA, 455 bp, Homo sapiens
aataataataatgcccggggctttattatgctgtatcactgctcagaggttaataatcctcactaactatcctatcaaatttgcaactggcagtttactctgatgattcaactccttttctatctacccccataatcccaccttactgatacacctcactggttactggcaagatacgctggatccctccagccttcttgctttccctgcaccagcccttcctcactttgccttgccctcaaagctaacaccacttaaaccacttaactgcattctgccattgtgcaaaagtctatgaaatgtttaggtttctttaaaggatcacagctctcatgagataacacccctccatcatgggacagacacttcaagcttctttttttgtaacccttcccacaggtcttagaacatgatgaccactcccccagctgccactgggggcagggatggtctgg

SEQ ID NO: 107 - DNA, 515 bp, Homo sapiens
aacccttcccacaggtcttagaacatgatgaccactcccccagctgccactgcggggcagggatggtctgcacaaggtctggtgctggctggcttcacttcctttgcacactcggaagcaggctgtccattaatgtctcggcattcttccagtcttctctgccaacccaattcacatgacttagaacattcgccccactcttcaatgacccatgctgaaaaagtggggatagcattgaaagattccttcttcttctttacgaagtaggtgtatttaattttaggtcgaagggcattgccacagtaagaacctggatggtcaagggctctttggagcaggctaaagctgcgaattctttccaatgccgcagaggagccgctgtacctcaagacaacacctttgtacataatgtcttgctctaaggtggacaaagtgtagtcaccataaagaatatatgtgccatcagcagcttttgatggcaggaagctgtcattgttcttggatccctctgttcc

SEQ ID NO: 108 - DNA, 359 bp, Homo sapiens
acttcgtaaagaagaagaaggaatctttcaatgctatccccactttttcagcatgggtcattgaagagtggggcgaatgttctaagtcatgtgaattgggttggcagagaagactggtagaatgccgagacattaatggacagcctgcttccgagtgtgcaaaggaagtgaagccagccagcaccagaccttgtgcagaccatccctgcccccagtggcagctgggggaagtggtcatcatgttctaagacctgcgggaagggttacaaaaaaagaagctttgaagtgtcttgtcccatgatggaggggtgttatctcattgagagctgtgatcctttaaagaaacctaaacatttcat

SEQ ID NO: 109 - DNA, 320 bp, Homo sapiens; identical to SEQ ID NO: 15

SEQ ID NO: 110 - DNA, 316 bp, Homo sapiens; identical to SEQ ID NO: 16

SEQ ID NO: 111 - DNA, 318 bp, Homo sapiens
  Misc_feature (4): N is any nucleic acid
  Misc_feature (6): N is any nucleic acid
  Misc_feature (261): N is any nucleic acid
agantnccgagacattaatggacagcctgcttccgagtgtgcaaaggaagtgaagccagccagcaccagaccttgtgcagaccatccctgcccccagtggcagctgggggagtggtcatcatgttctaagacctgtgggaagggttacaaaaaaagaagcttgaagtgtctgtcccatgatggaggggtgttatctcatgagagctgtgatcctttaaagaaacctaaacatttcatagacttttgcacaatggcagaatncagttaagtggtttaagtggtgttagctttgagggcaaggcaaagtgaggaagggct

SEQ ID NO: 112 - DNA, 314 bp, Homo sapiens
ttttttttctaaacttgtaatagatgtaacaaaagaaataataataataatgcccggggctttattatgctatatcactgctcagaggttaataatcctcactaactatcctatcaaatttgcaactggcagtttactctgatgattcaactccttttctatctacccccataatcccaccttactgatacacctcactggttactggcaagatacgctggatccctccagccttcttgctttccctgcaccagcccttcctcactttgccttgccctcaaagctaacaccacttaaaccacttaactgcattc

SEQ ID NO: 113 - DNA, 316 bp, Homo sapiens
aaggaatccttcaatgctatccccactgtttcagcatgggtcattgaagagtggggcgaatgttctaagtcatgtgaattgggttggcagaaaagacttgtagaatgccgagacattaatggacagcctgcgtccgagtgtgcaaaggaagtgaagccagccagcaccagaccttgtgcagaccatccctgcccccagtggcagctggggggagtggtcatcatgttctaagacctgtgggaaggggtacaaaaaaagaggcgtgaagtgtctgtcccatgatggaggggtttatctcatgagaactgtgatcctt

SEQ ID NO: 114 - DNA, 265 bp, Homo sapiens
  Misc_feature (10): N is any nucleic acid
  Misc_feature (11): N is any nucleic acid
  Misc_feature (15): N is any nucleic acid
  Misc_feature (97): N is any nucleic acid
  Misc_feature (231): N is any nucleic acid
  Misc_feature (249): N is any nucleic acid
agcagtttanncctntcaaagagcccttgaccatccaggttcttactgtgggcaatgcccttcgacctaaaattaaatacacctacttcgtaaagangaagaaggaatctttcaatgctatccccactttttcagcatgggtcattgaagagtggggcgaatgttctaagtcatgtgaattgggttggcagagaagactggtagaatgccgagacattaatggacagcctncttccgagtgtgcaaagnaagtgaagccagccag

SEQ ID NO: 115 - DNA, 334 bp, Mus musculus
cgtttgtggaggaaacggttccacatgcaagaagatgtcaggaatagtcactagtacaagacctgggtatcatgacattgtcacaattcctgctggagccaccaacattgaagtgaaacatcggaatcaaagggggtccagaaacaatggcagctttctggctattagagccgctgatggtacctatattctgaatggaaacttcactctgtccacactagagcaagacctcacctacaaaggtactgtcttaaggtacagtggttcctcggctgcgctggagagaatccgcagctttagtccactcaaagaacccttaaccatccaggttctt

SEQ ID NO: 116 - DNA, 528 bp, Mus musculus
agaattcctggatgatggtcatggtaattgcttccgtggtaggtctagcaaacaattaccatgaccatcatccaggaattctgtgatggtggctgacgtgcatttggaccagggcttggatgcatcgatgctggtaaggattgaagacattaaacgcttgtcttctgtagtaccgaagttctcttcacagaatttggaatcgtcatgagaaaggccaagtagatgcccaatttcatgagccacagtgaaggctgcatggaggccatcatcttcaatcactgcacagctgcgctccggagaacatatggtcccaacgtctgccattcccagggtgtcacatgaatgatgcccacataaatcctctcgggtgaacaggatggctgcatcgtagtgctcttcgtgatcatcccctagctggttatgttggtgctgccatttgcaaaagttcttgagggtcgtggccgcattcttgctcacctccagactcgtgtccttgtccgtcagcaccaccaccttcaccaccgccag

SEQ ID NO: 117 - DNA, 438 bp, Homo sapiens
  Misc_feature (389): N is any nucleic acid
  Misc_feature (432): N is any nucleic acid
atttgataggatagttagtgaggattattaacctctgagcagtgatatagcataataaagccccgggcattattattattatttcttttgttacatctattacaagtttagaaaaaacaaagcaattgtcaaaaaaagttagaactattacaacccctgtttcctggtacttatcaaatacttagtatcatgggggttgggaaatgaaaagtaggagaaaagtgagattttactaagacctgttttactttacctcactaacaatggggggagaaaggagtacaaataggatctttgaccagcactgtttatgggctgctatgggtttcagaggaatgtttatacattatttctacccgaggatttaaaacttcagattgttccaaccnggaggggaagggcttccggccaacgtggaattaaccggcaatnggcctt

SEQ ID NO: 118 - DNA, 455 bp, Homo sapiens
  Misc_feature (452): N is any nucleic acid
atttgataggatagttagtgaggattattaacctctgagcagtgatatagcataataaagccccgggcattattattattatttcttttgttacatctattacaagtttagaaaaaacaaagcaattgtcaaaaaaagttagaactattacaacccctgtttcctggtacttatcaaatacttagtatcatgggggttgggaaatgaaaagtaggagaaaagtgagattttactaagacctgttttactttacctcactaacaatggggggagaaaggagtacaaataggatctttgaccagcactgtttatggctgctatggtttcagagaatgtttatacattatttctaccgaggattaaaacttccagattgtttcaacatggagaggaaaggctcaggcaacgtggaaataacgcaaatgggcttcctcttttcctttttgggaccntct

SEQ ID NO: 119 - DNA, 380 bp, Homo sapiens
  Misc_feature (25): N is any nucleic acid
  Misc_feature (85): N is any nucleic acid
  Misc_feature (190): N is any nucleic acid
  Misc_feature (295): N is any nucleic acid
  Misc_feature (361): N is any nucleic acid
aatttgataggatagttagtgaggnttattaacctctgagcagtgatatagcataataaagccccgggcattattattattattncttttgttacatctattacaagtttagaaaaaacaaagcaattgtcaaaaaaagttagaactattacaacccctgtttcctggtacttatcaaatacttagtatnatgggggttgggaaatgaaaagtaggagaaaagtgagattttactaagacctgttttactttacctcactaacaatggggggagaaaggagtacanataggatctttgaccagcactgtttatggctgctatggtttcagaggaatgtttatacattatttctaccgaganttaaaacttcagattgttc

SEQ ID NO: 120 - DNA, 199 bp, Mus musculus
caatggcagcttgctggctataatagccgctgatggtacctatatactgaatggaaacttcactctgtccacactagagcaagacctcacctacgaatgtactgtcttaaggtacagtggttcctcggctgcgcaggaaagagtccgcagctttagtccactcaaataacccttaaccatccaggttcttatggtagga

SEQ ID NO: 121 - DNA, 439 bp, Homo sapiens
  Misc_feature (198): N is any nucleic acid
  Misc_feature (199): N is any nucleic acid
  Misc_feature (203): N is any nucleic acid
atttaacctctgagcagtgatatagcataataaagccccgggcattattattattatttcttttgttacatctattacaagtttagaaaaaacaaagcaattgtcaaaaaaagttagaactattacaacccctgtttcctggtacttatcaaatacttagtatcatgggggttgggaaatgaaaagtaggaggaaagnngagnttttactaagacctgttttacctttacctcactaacaatggggggagaaaggagtacaaataggatctttgaccagcactgtttatggctgctatggtttcagagaatgtttatacattatttctaccgagaattaaaacttcagattgttcaacatggagagaaaggctcagcaacgtggaaataacgcaaatgggcttccccctttcccttttttgggaccatctcaggtcctt

SEQ ID NO: 122 - DNA, 471 bp, Homo sapiens
cagagtaaactgccagttgcaaatttgataggatagttagtgaggattattaacctctgagcagtgatatagcataataaagccccgggcattattattattattatttcttttgttacatctattacaagtttagaaaaaacaaagcaattgtcaaaaaaagttagaactattacaacccctgtttcctggtacttatcaaatacttagtatcatgggggttgggaaatgaaaagtaggagaaaagtgagattttactaagacctgttttacttttcctcactaacaatggggggagaaaggagtacaaataggatctttgaccagcactgtttatggctgctatggtttcagagaatgtttatacattatttctacccgagaattaaaacttcagattggttcaacatgagagaaaggctccagcaacgtgaaattaacgccaatggcttcctccttcccttttttgga

SEQ ID NO: 123 - DNA, 424 bp, Homo sapiens
  Misc_feature (39): N is any nucleic acid
  Misc_feature (51): N is any nucleic acid
  Misc_feature (395): N is any nucleic acid
cgtgaggattattaacctctgagcagtgatatagcatantaaagccccggnattattattattatttcttttgttacatctattacaagtttagaaaaaacaaagcaattgtcaaaaaaagttagaactattacaacccctgtttcctggtacttatcaaatacttagtatcatgggggttgggaaatgaaaagtaggagaaaagtgagattttactaagacctgttttactttacctcactaacaatggggggagaaaggagtacaaataggatctttgaccagcactgtttatggctgctaatggtttcagagaatgtttatacattatttctacccgagaattaaaacttcagattgttcaacctgagagaaaggctcagcaacgtgaaatnacgccaatggcttcctctttccctttttg

SEQ ID NO: 124 - DNA, 458 bp, Homo sapiens
  Misc_feature (453): N is any nucleic acid
tacatctattacaagtttagaaaaaacaaagcaattgtcaaaaaaagttagaactattacaacccctgtttcctggtacttatcaaatacttagtatcatgggggttgggaaatgaaaagtaggagaaaagtgagattttactaagacctgttttactttacctcactaacaatggggggagaaaggagtacaaataggatctttgaccagcactgtttatggctgctatggtttcagagaatgtttatacattatttctaccgagaattaaaacttcagattgttcaacatgagagaaaggctcagcaacgtgaaataacgcaaatggcttcctctttccttttttggaccacagccagccttggtctccttgcagtggctacatgattacatcatttctggataatagtcatggggaatgtttgatggacaagctcagaatcccatacagntccca

SEQ ID NO: 125 - DNA, 4014 bp, Homo sapiens
  CDS (466)..(3366)
cccacgcgtccgcccacgcgtccggcggctccgagccaggggctattgcaaagccagggtgcgctaccggacggagaggggagagccctgagcagagtgagcaacatcgcagccaaggcggaggccgaagaggggcgccaggcaccaatctccgcgttgcctcagccccggaggcgccccagagcgcttcttgtcccagcagagccactctgcctgcgcctgcctctcagtgtctccaactttgcgctggaagaaaaacttcccgcgcgccggcagaactgcagcgcctcctcttagtgactccgggagcttcggctgtagccggctctgcgcgcccttccaacgaataatagaaattgttaattttaacaatccagagcaggccaacgaggctttgctctcccgacccgaactaaagctccctcgctccgtgcgctgctacgagcggtgtctcctggggctccaatgcagcgagctgtgcccgaggggttcggaaggcgcaagctgggcagcgacatggggaacgcggagcgggctccggggtctcggagctttgggcccgtacccacgctgctgctgctcgccgcggcgctactggccgtgtcggacgcactcgggcgcccctccgaggaggacgaggagctagtggtgccggagctggagcgcgtcccgggacacgggaccacgcgcctccgcctgcacgcctttgaccagcagctggatctggacgtgccgcccgacagcagctttttggcgcccggcttcacgctccagaacgtggggcgcaaatccgggtccgacaccccgcttccggaaaccgacctggcgcactgcttctactccggcaccgtgaatggcgatcccagctcggctgccgccctcagcctctgcgagggcgtgcgcggcgccttctacctgctgggggaggcgtatttcatccagccgctgcccgccgccagcgagcgcctcgccaccgccgccccaggggagaagccgccggcaccactacagttccacctcctgcggcggaatcggcagggcgacgtaggcggcacgtgcggggtcgtggacgacgagccccggccgactgggaaagcggagaccgaagacgaggacgaagggactgagggcgaggacgaagggcctcagtggtcgccgcaggacccggcactgcaaggcgtaggacagcccacaggaactggaagcataagaaagaagcgatttgtgtccagtcaccgctatgtggaaaccatgcttgtggcagaccagtcgatggcagaattccacggcagtggtctaaagcattaccttctcacgttgttttcggtggcagccagattgtacaaacaccccagcattcgtaattcagttagcctggtggtggtgaagatcttggtcatccacgatgaacagaaggggcccgaagtgacctccaatgctgccctcactctgcggaacttttgcaactggcagaagcagcacaacccacccagtgaccgggatgcagagcactatgacacagcaattcttttcaccagacaggacttgtgtgggtcccagacatgtgatactcttgggatggctgatgttggaactgtgtgtgatccgagcagaagctgctccgtcatagaagatgatggtttacaagctgccttcaccacagcccatgaattaggccacgtgtttaacatgccacatgatgatgcaaagcagtgtgccagccttaatggtgtgaaccaggattcccacatgatggcgtcaatgctttccaacctggaccacagccagccttggtctccttgcagtggctacatgattacatcatttctggataatggtcatggggaatgtttgatggacaagcctcagaatcccatacagctcccaggcgatctccctggcacctcgtacgatgccaaccggcagtgccagtttacatttggggaggactccaaacactgccctgatgcagccagcacatgtagcaccttgtggtgtaccggcacctctggtggggtgctggtgtgtcaaaccaaacacttcccgtgggcggatggcaccagctgtggagaagggaaatggtgtatcaacggcaagtgtgtgaacaaaaaccacagaaagcattttgatacgccttttcatggaagctggggaatgtgggggccttggggagactgttcgagaacgtgcggtggaggagtccagtacacgatgagggaatgtgacaacccagtcccaaagaatggagggaagtactgtgaaggcaaacgagtgcgctacagatcctgtaaccttgaggactgtccagacaataatggaaaaacctttagagaggaacaatgtgaagcacacaacgagttttcaaaagcttcctttgggagtgggcctgcggtggaatggattcccaagtacgctggcgtctcaccaaaggacaggtgcaagctcatctgccaagccaaaggcattggctacttcttcgttttgcagcccaaggttgtagatggtactccatgtagcccagattccacctctgtctgtgtgcaaggacagtgtgtaaaagctggttgtgatcgcatcatagactccaaaaagaagtttgataaatgtggtgtttgcgggggaaatggatctacttgtaaaaaaatatcaggatcagttactagtgcaaaacctggatatcatgatatcatcacaattccaactggagccaccaacatcgaagtgaaacagcggaaccagaggggatccaggaacaatggcagctttcttgccatcaaagctgctgatggcacatatattcttaatggtgactacactttgtccaccttagagcaagacattatgtacaaaggtgttgtcttgaggtacagcggctcctctgcggcattggaaagaattcgcagctttagccctctcaaagagcccttgaccatccaggttcttactgtgggcaatgcccttcgacctaaaattaaatacacctacttcgtaaagaagaagaaggaatctttcaatgctatccccactttttcagcatgggtcattgaagagtggggcgaatgttctaagtcatgtgaattgggttggcagagaagactggtagaatgccgagacattaatggacagcctgcttccgagtgtgcaaaggaagtgaagccagccagcaccagaccttgtgcagaccatccctgcccccagtggcagctgggggagtggtcatcatgttctaagacctgtgggaagggttacaaaaaaacaagcttgaagtgtctgtcccatgatggaggggtgttatctcatgacagctgtgatcctttaaagaaacctaaacatttcatagacttttgcacaatggcagaatgcagttaagtggtttaagtggtgttagctttgagggcaaggcaaagtgaggaagggctggtgcagggaaagcaagaaggctggagggatccagcgtatcttcccagtaaccagtgaggtgtatcagtaaggtgggattatgggggtagatagaaaaggagttgaatcatcagagtaaactgccagttgcaaatttgataggatagttagtgaggattattaacctctgagcagtgatatagcataataaagccccgggcattattattattatttcttttgttacatctactacaagtttagaaaaaacaaagcaattgtcaaaaaaagttagaactattacaacccctgcttcctggtacttatcaaatacttagtatcatgggggttgggaaatgaaaagtaggagaaaagtgagattttactaagacctgttttactttacctcactaaacaatggggggagaaaggagtacaaataggatcttttgaccagcactgtttatgggctgctatggtttcagagaacgtctatacattatttctaccgaggatttaaaacttccagattgttccaacatggagaggaaaggctcaggcaacgtggaaataacgcaatgggcttcccccttccctttttgggacccactccag

SEQ ID NO: 126 - protein, 967 aa, ITGL-TSP
MQRAVPEGFGRRKLGSDMGNAERAPGSRSFGPVPTLLLLAAALLAVSDALGRPSEEDEELVVPELERVPGHGTTRLRLHAFDQQLDLDVPPDSSFLAPGFTLQNVGRKSGSDTPLPETDLAHCFYSGTVNGDPSSAAALSLCEGVRGAFYLLGEAYFIQPLPAASERLATAAPGEKPPAPLQFHLLRRNRQGDVGGTCGVVDDEPRPTGKAETEDEDEGTEGEDEGPQWSPQDPALQGVGQPTGTGSIRKKRFVSSHRYVETMLVADQSMAEFHGSGLKHYLLTLFSVAARLYKHPSIRNSVSLVVVKILVIHDEQKGPEVTSNAALTLRNFCNWQKQHNPPSDRDAEHYDTAILFTRQDLCGSQTCDTLGMADVGTVCDPSRSCSVIEDDGLQAAFTTAHELGHVFNMPHDDAKQCASLNGVNQDSHMMASMLSNLDHSQPWSPCSGYMITSFLDNGHGECLMDKPQNPIQLPGDLPGTSYDANRQCQFTFGEDSKHCPDAASTCSTLWCTGTSGGVLVCQTKHFPWADGTSCGEGKWCINGKCVNKNHRKHFDTPFHGSWGMWGPWGDCSRTCGGGVQYTMRECDNPVPKNGGKYCEGKRVRYRSCNLEDCPDNNGKTFREEQCEAHNEFSKASFGSGPAVEWIPKYAGVSPKDRCKLICQAKGIGYFFVLQPKVVDGTPCSPDSTSVCVQGQCVKAGCDRIIDSKKKFDKCGVCGGNGSTCKKISGSVTSAKPGYHDIITIPTGATNIEVKQRNQRGSRNNGSFLAIKAADGTYILNGDYTLSTLEQDIMYKGVVLRYSGSSAALERIRSFSPLKEPLTIQVLTVGNALRPKIKYTYFVKKKKESFNAIPTFSAWVIEEWGECSKSCELGWQRRLVECRDINGQPASECAKEVKPASTRPCADHPCPQWQLGEWSSCSKTCGKGYKKTSLKCLSHDGGVLSHDSCDPLKKPKHFIDFCTMAECS

(19) United States
(12) Patent Application Publication
(10) Pub. No.: US 2004/0002449 A1
(43) Pub. Date: Jan. 1, 2004

(54) METH1 and METH2 polynucleotides and polypeptides

(75) Inventors: Luisa Iruela-Arispe, Los Angeles, CA (US); Gregg A. Hastings, Westlake Village, CA (US); Steven M. Ruben, Olney, MD (US); Zdenka L. Jonak, Devon, PA (US); Stephen H. Trulli, Havertown, PA (US); James A. Fornwald, Norristown, PA (US); Jonathan A. Terrett, Oxfordshire (GB)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Human Genome Sciences, Inc.

(21) Appl. No.: 09/989,687

(22) Filed: Nov. 21, 2001

Related U.S. Application Data
(63) Continuation-in-part of application No. PCT/US00/14462, filed on May 25, 2000.
(63) Continuation-in-part of application No. 09/318,208, filed on May 25, 1999, now abandoned.
(63) Continuation-in-part of application No. 09/373,658, filed on Aug. 13, 1999.
(60) Provisional application No. 60/171,503, filed on Dec. 22, 1999.
(60) Provisional application No. 60/183,792, filed on Feb. 22, 2000.
(60) Provisional application No. 60/144,882, filed on Jul. 20, 1999.
(60) Provisional application No. 60/147,823, filed on Aug. 10, 1999.

(51) Int. Cl. A61K 38/17; A61K 48/00

(52) U.S. Cl. 514/12; 514/44

(57) ABSTRACT

The present invention relates to novel anti-angiogenic proteins, related to thrombospondin. More specifically, isolated nucleic acid molecules are provided encoding human METH1 and METH2. METH1 and METH2 polypeptides are also provided, as are vectors, host cells and recombinant methods for producing the same. Also provided are diagnostic methods for the prognosis of cancer and therapeutic methods for treating individuals in need of an increased amount of METH1 or METH2. Also provided are methods for inhibiting angiogenesis using METH1 or METH2.

### CROSS-REFERENCE TO RELATED APPLICATIONS

This application is a continuation-in-part of International Appl. No. PCT/US00/14462, filed May 25, 2000, published in English, the disclosure of which is incorporated by reference herein; said Appl. No. PCT/US00/14462 claims priority benefit to U.S. Provisional Appl. No. 60/171,503, filed Dec. 22, 1999 and U.S. Provisional Appl. No. 60/183,792, filed Feb. 22, 2000, the disclosures of both of which are incorporated by reference herein; said Appl. No. PCT/US00/14462 is also a continuation-in-part of U.S. Appl. No. 09/318,208, filed May 25, 1999, the disclosure of which is incorporated by reference herein; said Appl. No. PCT/US00/14462 is also a continuation in part of U.S. Appl. No. 09/373,658, filed Aug. 13, 1999, the disclosure of which is incorporated by reference herein; said Appl. No. 09/373,658 claims priority benefit of U.S. Provisional Appl. No. 60/144,882, filed Jul. 20, 1999 and U.S. Provisional Appl. No. 60/147,823, filed Aug. 10, 1999, the disclosures of both of which are incorporated by reference herein.

### STATEMENT AS TO RIGHTS TO INVENTIONS MADE UNDER FEDERALLY-SPONSORED RESEARCH AND DEVELOPMENT

Part of the work performed during development of this invention utilized U.S. Government funds. The U.S. Government may have certain rights in this invention.

### BACKGROUND OF THE INVENTION

1. Field of the Invention

The present invention relates to novel anti-angiogenic proteins, related to thrombospondin. More specifically, isolated nucleic acid molecules are provided encoding human METH1 and METH2 (ME, for metalloprotease, and TH, for thrombospondin). METH1 and METH2 polypeptides are also provided, as are vectors, host cells and recombinant methods for producing the same. Also provided are diagnostic methods for the prognosis of cancer and therapeutic methods for treating individuals in need of an increased amount of METH1 or METH2. Also provided are methods for inhibiting angiogenesis using METH1 or METH2.

2. Related Art

Angiogenesis, the formation of new blood vessels from pre-existing vasculature, is a tightly regulated process in normal adults. Under physiological circumstances, growth of new capillaries is tightly controlled by an interplay of growth regulatory proteins which act either to stimulate or to inhibit blood vessel growth. Normally, the balance between these forces is tipped in favor of inhibition and consequently blood vessel growth is restrained. Under certain pathological circumstances, however, local inhibitory controls are unable to restrain the increased activity of angiogenic inducers. Angiogenesis is a key step in the metastasis of cancer (Folkman, Nature Med. 1:27-31 (1995)) and in abnormal wound healing, inflammation, rheumatoid arthritis, psoriasis, and diabetic retinopathy, it is integral to the pathology (Folkman et al., Science 235:442-447 (1987)), engendering the hope that these pathological entities could be regulated by pharmacological and/or genetic suppression of blood vessel growth (Iruela-Arispe et al., Thromb. Haem. 78:672-677 1997)).

Thrombospondin-1 (TSP-1) is a 450 kDa, anti-angiogenic adhesive glycoprotein released from activated platelets and secreted by growing cells (reviewed in Adams, Int. J. Biochem. Cell. Biol. 29:861-865 (1997)). TSP-1 is a homotrimer, with each subunit comprised of a 1152 amino acid residue polypeptide, post-translationally modified by N-linked glycosylation and beta-hydroxylation of asparagine residues.

TSP-1 protein and mRNA levels are regulated by a variety of factors. TSP-1 protein levels are downregulated by IL-1 alpha and TNF alpha. TSP-1 mRNA and protein levels are upregulated by polypeptide growth factors including PDGF, TGF-beta, and bFGF (Bornstein, Faseb J. 6:3290-3299 (1992)) and are also regulated by the level of expression of the p53 tumor suppressor gene product (Dameron et al., Science 265:1582-1584 (1994)). At least four other members of the thrombospondin family have been identified: TSP-2, TSP-3, TSP-4, and TSP-5 (also called COMP). There is a need in the art to identify other molecules involved in the regulation of angiogenesis.

### SUMMARY OF THE INVENTION

The present invention provides isolated nucleic acid molecules comprising a polynucleotide encoding the METH1 polypeptide having the amino acid sequence shown in SEQ ID NO:2 or the amino acid sequence encoded by the cDNA clone deposited in a bacterial host as ATCC Deposit Number 209581 on Jan. 15, 1998.

The present invention also provides isolated nucleic acid molecules comprising a polynucleotide encoding the METH2 polypeptide having the amino acid sequence shown in SEQ 1D NO:4 or the amino acid sequence encoded by the cDNA clone deposited in a bacterial host as ATCC Deposit No.209582 on Jan. 15, 1998 or ATCC Deposit No. PTA 1478 on Mar. 14, 2000.

The present invention also relates to recombinant vectors, which include the isolated nucleic acid molecules of the present invention, and to host cells containing the recombinant vectors, as well as to methods of making such vectors and host cells and for using them for production of METH1 or METH2 polypeptides or peptides by recombinant techniques.

The invention further provides an isolated METH1 or METH2 polypeptide having an amino acid sequence encoded by a polynucleotide described herein.

The invention further provides a diagnostic method useful during diagnosis or prognosis of cancer.

An additional aspect of the invention is related to a method for treating an individual in need of an increased level of METH1 or METH2 activity in the body comprising administering to such an individual a composition comprising a therapeutically effective amount of an isolated METH1 or METH2 polypeptide of the invention or an agonist thereof.

### BRIEF DESCRIPTION OF THE FIGURES

FIGS. 1A-C show the nucleotide (SEQ ID NO: 1) and deduced amino acid (SEQ ID NO:2) sequences of METH1. The protein has a predicted leader sequence of about 28 amino acid residues (underlined).

FIGS. 2A-B show the nucleotide (SEQ ID NO:3) and deduced amino acid (SEQ ID NO:4) sequences of METH2. The protein has a predicted leader sequence of about 23 amino acid residues (underlined).

FIGS. 3A-C show a comparison of the amino acid sequence of METH1 (SEQ ID NO:2) and METH2 (SEQ ID NO:4) with that of their closest homologue, a bovine metalloprotease (pNPI) (SEQ ID NO:5). Identical amino acids are boxed. Functional domains predicted by sequence and structural homology are labeled, including the signal peptide (single line), the potential cleavage site for mammalian subtilisin (double underlined), the zinc-binding-site (dotted line; amino acids 383-395 in METH1 and 363-375 in METH2) in the metalloprotease domain, and the putative disintegrin loops (arrows).

FIG. 4 shows the primary structure of METH1, METH2 and pNPI which includes a prodomain, a catalytic metalloprotease domain, a cysteine rich disintegrin domain, a TSP-like domain, a spacer region and a different number of TSP-like domains, three for METH1, two for METH2, and four for pNPI.

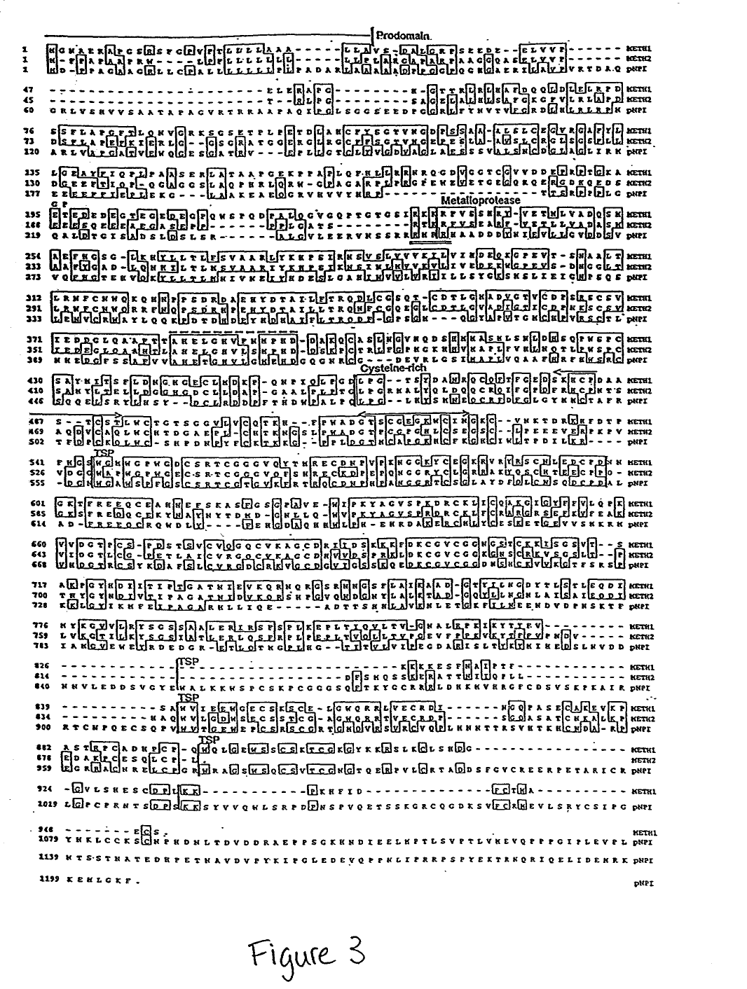

FIG. 5 shows a comparison of the TSP-like domain of METH1 (SEQ ID NO:2) and METH2 (SEQ ID NO:4) with those of TSP1 (SEQ ID NOs:6, 7, and 8) and TSP2 (SEQ ID NOs:9, 10, and 11), cysteines are numbered 1 to 6, tryptophans are marked by asterisks.

FIGS. 6A-6D show that peptides and recombinant protein derived from the TSP-like domain of METH1 and METH2 block VEGF-induced angiogenesis. Angiogenesis was induced on CAMs from 12-14-day-old embryos using a nylon mesh containing VEGF casted on matrigel and in the presence or absence of the peptides or recombinant protein. Capillary density was evaluated as described in Example 4. Positive and negative control included VEGF alone and vehicle alone, respectively. (A) Quantification of the angiogenic response induced by VEGF in the presence of recombinant proteins. TSP1, purified platelet TSP1, GST, purified GST, GST-TSP1, GST-METH1, and GST-METH2 are described in Example 4. (B) Quantification of the angiogenic response induced by VEGF in the presence or absence of the peptides; P—TSP1, P-METH1, and P-METH2 (peptide derived from the Type I repeats of TSP, METH1 and METH2, respectively); SC1 and SC2are scramble peptides used as controls. (C) Dose-response of the VEGF-induced angiogenesis in the presence of GST-METH1. (D) Dose-response of the VEGF-induced angiogenesis in the presence of GST-METH2. The angiogenic index was expressed considering the vascular response from the VEGF-matrigel as 100% and subtracting the background levels (matrigel alone). Assays were repeated, at least, twice. Each treatment was done in triplicate. Values represent the mean, bars indicate standard deviations. *p<0.001.

FIGS. 7A-E show the effect of METH1 and METH2 recombinant proteins on bFGF-stimulated cell proliferation. Cells were cultured on 24-well plates in media containing bFGF and the recombinant protein to be tested (3 μg/ml, unless indicated in the graph). Controls included vehicle or GST recombinant protein alone. (A), HDEC, human dermal endothelial cells; (13), HMEC, human mammary epithelial cells; (C), HDF, human dermal fibroblasts; (D), SMC, smooth muscle cells; (E) Dose-response of GST-METH1 and GST-METH2 on HDEC proliferation. Experiments were repeated, at least, twice. Each treatment was done in triplicate. Values represent the mean, bars indicate standard deviations. *p<0.01.

FIG. 8 shows a schematic representation of the pHE4-5 expression vector (SEQ ID NO: 12) and the subcloned METH1 or METH2 cDNA coding sequence. The locations of the kanamycin resistance marker gene, the METH1 or METH2 coding sequence, the oriC sequence, and the lacIq coding sequence are indicated.

FIG. 9 shows the nucleotide sequence of the regulatory elements of the pHE promoter (SEQ ID NO:13). The two lac operator sequences, the Shine-Delgarno sequence (S/D), and the terminal HindIII and NdeI restriction sites (italicized) are indicated.

FIG. 10 shows an analysis of the METH1 amino acid sequence. Alpha, beta, turn and coil regions; hydrophilicity and hydrophobicity; amphipathic regions; flexible regions; antigenic index and surface probability are shown, and all were generated using the default settings. In the “Antigenic Index or Jameson-Wolf” graph, the positive peaks indicate locations of the highly antigenic regions of the METH1 or METH2 protein, i.e., regions from which epitope-bearing peptides of the invention can be obtained. The domains defined by these graphs are contemplated by the present invention. Tabular representation of the data summarized graphically in FIG. 10 can be found in Table 1.

FIG. 11 shows an analysis of the METH2 amino acid sequence. Alpha, beta, turn and coil regions; hydrophilicity and hydrophobicity; amphipathic regions; flexible regions; antigenic index and surface probability are shown, and all were generated using the default settings. In the “Antigenic Index or Jameson-Wolf” graph, the positive peaks indicate locations of the highly antigenic regions of the METH1 or METH2 protein, i.e., regions from which epitope-bearing peptides of the invention can be obtained. The domains defined by these graphs are contemplated by the present invention. Tabular representation of the data summarized graphically in FIG. 11 can be found in Table 2.

TABLE 1

| | | Garni . . . | Chou- . . . | Garni . . . | Chou- . . . | Garni . . . | Chou- . . . | Garni . . . | Kyte- . . . | Eisen . . . | Eisen . . . | Karpl . . . | James . . . | Emini |
|---|---|---|---|---|---|---|---|---|---|---|---|---|---|---|
| Res | Pos. | Alpha | Alpha | Beta | Beta | Turn | Turn | Coil | Hydro . . . | Alpha | Beta | Flexi . . . | Antig . . . | Surfa . . . |
| Met | 1 | A | A | . | . | . | . | . | 0.41 | * | . | . | −0.30 | 0.60 |
| Gly | 2 | . | A | . | . | . | . | C | 0.91 | * | . | . | 0.50 | 0.81 |
| Asn | 3 | A | A | . | . | . | . | . | 0.71 | * | . | . | 0.75 | 1.24 |
| Ala | 4 | A | A | . | . | . | . | . | 0.89 | * | . | . | 1.09 | 1.26 |
| Glu | 5 | A | A | . | . | . | . | . | 0.93 | * | . | F | 1.58 | 1.97 |
| Arg | 6 | . | A | B | . | . | . | . | 1.23 | . | . | F | 1.92 | 1.21 |
| Ala | 7 | . | . | B | . | . | T | . | 1.69 | . | . | F | 2.66 | 1.61 |
| Pro | 8 | . | . | . | . | T | T | . | 1.39 | . | . | F | 3.40 | 1.82 |
| Gly | 9 | . | . | . | . | T | T | . | 1.28 | . | . | F | 3.06 | 1.25 |
| Ser | 10 | . | . | . | . | T | T | . | 0.93 | . | . | F | 2.42 | 1.07 |
| Arg | 11 | . | . | . | . | T | T | . | 0.61 | . | * | F | 1.93 | 0.68 |
| Ser | 12 | . | . | . | . | T | T | . | 0.34 | * | . | F | 1.74 | 1.07 |
| Phe | 13 | . | . | B | . | . | T | . | 0.34 | * | . | F | 0.25 | 0.59 |
| Gly | 14 | . | . | B | . | . | T | . | 0.38 | * | . | F | 0.25 | 0.47 |
| Pro | 15 | . | . | B | B | . | . | . | −0.13 | * | . | F | −0.45 | 0.50 |
| Val | 16 | . | . | B | B | . | . | . | −1.06 | * | . | F | −0.45 | 0.48 |
| Pro | 17 | . | . | B | B | . | . | . | −1.57 | . | . | F | −0.45 | 0.40 |
| Thr | 18 | . | A | B | . | . | . | . | −1.68 | . | . | F | −0.45 | 0.21 |
| Leu | 19 | . | A | B | . | . | . | . | −1.92 | . | . | . | −0.60 | 0.24 |
| Leu | 20 | A | A | . | . | . | . | . | −2.30 | . | . | . | −0.60 | 0.15 |
| Leu | 21 | A | A | . | . | . | . | . | −2.03 | . | . | . | −0.60 | 0.11 |
| Leu | 22 | A | A | . | . | . | . | . | −2.63 | . | . | . | −0.60 | 0.13 |
| Ala | 23 | A | A | . | . | . | . | . | −3.13 | . | . | . | −0.60 | 0.13 |
| Ala | 24 | A | A | . | . | . | . | . | −2.91 | . | . | . | −0.60 | 0.13 |
| Ala | 25 | A | A | . | . | . | . | . | −2.96 | . | . | . | −0.60 | 0.16 |
| Leu | 26 | A | A | . | B | . | . | . | −2.44 | . | . | . | −0.60 | 0.12 |
| Leu | 27 | A | A | . | B | . | . | . | −1.63 | . | . | . | −0.60 | 0.16 |
| Ala | 28 | A | A | . | B | . | . | . | −1.63 | . | . | . | −0.30 | 0.26 |
| Val | 29 | A | A | . | B | . | . | . | −1.86 | . | . | . | −0.30 | 0.32 |
| Ser | 30 | A | A | . | . | . | . | . | −1.61 | * | * | . | −0.30 | 0.32 |
| Asp | 31 | A | A | . | . | . | . | . | −0.69 | * | * | F | −0.15 | 0.31 |
| Ala | 32 | A | A | . | . | . | . | . | −0.09 | | * | F | 0.75 | 0.83 |
| Leu | 33 | . | A | . | . | . | . | C | 0.20 | * | . | F | 1.55 | 0.96 |
| Gly | 34 | . | A | . | . | . | . | C | 1.06 | * | * | F | 1.85 | 0.77 |
| Arg | 35 | . | . | . | . | . | T | C | 1.36 | * | * | F | 2.70 | 1.32 |
| Pro | 36 | . | . | . | . | . | T | C | 1.36 | * | * | F | 3.00 | 2.76 |
| Ser | 37 | . | . | . | . | . | T | C | 1.94 | * | . | F | 2.70 | 4.66 |
| Glu | 38 | A | . | . | . | . | T | . | 2.76 | * | . | F | 2.20 | 4.12 |
| Glu | 39 | A | A | . | . | . | . | . | 2.29 | * | * | F | 1.50 | 4.61 |
| Asp | 40 | A | A | . | . | . | . | . | 1.32 | * | * | F | 1.20 | 2.84 |
| Glu | 41 | A | A | . | . | . | . | . | 0.68 | . | . | F | 0.90 | 1.22 |
| Glu | 42 | A | A | . | . | . | . | . | 0.77 | . | . | F | 0.75 | 0.52 |
| Leu | 43 | A | A | . | . | . | . | . | 0.77 | . | . | . | 0.60 | 0.48 |
| Val | 44 | A | A | . | . | . | . | . | −0.04 | . | . | . | 0.60 | 0.48 |
| Val | 45 | A | A | . | . | . | . | . | −0.04 | * | . | . | −0.30 | 0.23 |
| Pro | 46 | A | A | . | . | . | . | . | 0.07 | * | . | . | −0.30 | 0.48 |
| Glu | 47 | A | . | . | . | . | . | . | −0.52 | * | . | F | 1.10 | 1.27 |
| Leu | 48 | A | . | . | . | . | . | . | 0.08 | * | . | F | 1.41 | 1.73 |
| Glu | 49 | A | . | . | . | . | . | . | 0.59 | * | . | F | 1.72 | 1.73 |
| Arg | 50 | A | . | . | . | . | . | . | 1.41 | * | . | F | 1.88 | 0.99 |
| Ala | 51 | A | . | . | . | . | T | . | 1.28 | * | . | F | 2.24 | 1.64 |
| Pro | 52 | . | . | . | . | T | T | . | 0.97 | * | . | F | 3.10 | 0.93 |
| Gly | 53 | . | . | . | . | T | T | . | 1.47 | * | * | F | 2.49 | 0.69 |
| His | 54 | . | . | . | . | . | T | C | 1.58 | * | * | F | 1.38 | 0.98 |
| Gly | 55 | . | . | . | . | . | . | C | 0.66 | * | * | F | 1.62 | 1.25 |
| Thr | 56 | . | . | . | . | . | . | C | 1.36 | . | * | F | 0.71 | 1.04 |
| Thr | 57 | . | A | B | . | . | . | . | 0.76 | . | * | F | 0.60 | 1.49 |
| Arg | 58 | . | A | B | . | . | . | . | 1.07 | . | * | F | 0.60 | 1.25 |
| Leu | 59 | . | A | B | . | . | . | . | 0.51 | . | * | . | 0.45 | 1.17 |
| Arg | 60 | . | A | B | . | . | . | . | 0.16 | . | * | . | 0.30 | 0.82 |
| Leu | 61 | . | A | B | . | . | . | . | 0.47 | . | * | . | −0.30 | 0.36 |
| His | 62 | . | A | B | . | . | . | . | 0.78 | . | * | . | −0.30 | 0.74 |
| Ala | 63 | A | A | . | . | . | . | . | 0.67 | . | * | . | 0.30 | 0.65 |
| Phe | 64 | A | A | . | . | . | . | . | 0.67 | . | * | . | −0.15 | 1.37 |
| Asp | 65 | A | A | . | . | . | . | . | 0.56 | . | * | F | −0.15 | 0.83 |
| Gln | 66 | A | A | . | . | . | . | . | 0.56 | . | * | F | 0.60 | 1.37 |
| Gln | 67 | A | A | . | . | . | . | . | 0.59 | . | * | F | 0.60 | 1.30 |
| Leu | 68 | A | A | . | . | . | . | . | 0.37 | * | * | F | 0.90 | 1.35 |
| Asp | 69 | A | A | . | . | . | . | . | 1.18 | * | * | . | 0.30 | 0.64 |
| Leu | 70 | . | A | B | . | . | . | . | 0.97 | . | * | . | 0.94 | 0.73 |
| Glu | 71 | . | A | B | . | . | . | . | 0.97 | . | * | . | 1.43 | 1.37 |
| Leu | 72 | . | A | B | . | . | . | . | 0.67 | . | * | . | 1.77 | 1.37 |
| Arg | 73 | . | . | . | . | . | T | C | 1.18 | * | * | F | 2.86 | 2.22 |
| Pro | 74 | . | . | . | . | T | T | . | 0.48 | * | * | F | 3.40 | 1.72 |
| Asp | 75 | . | . | . | . | T | T | . | 0.48 | . | * | F | 2.76 | 1.80 |
| Ser | 76 | . | . | . | . | . | T | C | −0.11 | . | * | F | 2.07 | 0.76 |
| Ser | 77 | . | . | B | . | . | . | . | 0.49 | * | * | F | 0.73 | 0.50 |
| Phe | 78 | . | | B | . | . | . | . | 0.03 | * | * | . | 0.24 | 0.46 |
| Leu | 79 | . | . | B | . | . | . | . | −0.46 | . | . | . | −0.40 | 0.34 |
| Ala | 80 | . | . | B | . | . | T | . | −0.77 | . | . | . | −0.20 | 0.22 |
| Pro | 81 | . | . | B | . | . | T | . | −1.28 | . | . | . | −0.20 | 0.37 |
| Gly | 82 | . | . | . | . | T | T | . | −0.98 | . | . | . | 0.20 | 0.37 |
| Phe | 83 | . | . | B | . | . | T | . | −0.28 | . | . | . | −0.20 | 0.63 |
| Thr | 84 | . | . | B | B | . | . | . | −0.32 | . | . | . | −0.60 | 0.65 |
| Leu | 85 | . | . | B | B | . | . | . | −0.08 | * | * | . | −0.60 | 0.49 |
| Gln | 86 | . | . | B | B | . | . | . | 0.24 | * | . | . | −0.29 | 0.56 |
| Asn | 87 | . | . | B | . | . | T | . | 0.63 | * | . | F | 0.87 | 0.76 |
| Val | 88 | . | . | B | . | . | T | . | 1.03 | * | * | F | 1.93 | 1.84 |
| Gly | 89 | . | . | . | . | . | T | C | 1.00 | * | . | F | 2.74 | 1.42 |
| Arg | 90 | . | . | . | . | T | T | | 1.51 | * | . | F | 3.10 | 0.87 |
| Lys | 91 | . | . | . | . | . | T | C | 1.51 | * | . | F | 2.74 | 1.58 |
| Ser | 92 | . | . | . | . | . | T | C | 1.20 | * | . | F | 2.43 | 2.76 |
| Gly | 93 | . | . | . | . | . | T | C | 1.84 | . | . | F | 2.38 | 2.04 |
| Ser | 94 | . | . | . | . | . | T | C | 1.38 | . | . | F | 2.33 | 1.57 |
| Glu | 95 | . | . | . | . | . | . | C | 1.06 | . | . | F | 1.63 | 0.97 |
| Thr | 96 | . | . | . | . | . | . | C | 1.01 | . | . | F | 2.04 | 1.51 |
| Pro | 97 | . | . | . | . | . | . | C | 1.00 | . | . | F | 2.60 | 1.96 |
| Leu | 98 | . | . | . | . | . | . | C | 1.34 | . | . | F | 2.04 | 1.63 |
| Pro | 99 | A | . | . | . | . | . | . | 0.83 | . | . | F | 1.58 | 1.89 |
| Glu | 100 | A | A | . | . | . | . | . | 0.24 | . | . | F | 1.12 | 1.01 |
| Thr | 101 | A | A | . | . | . | . | . | 0.52 | . | . | F | 0.86 | 1.23 |
| Asp | 102 | A | A | . | . | . | . | . | 0.07 | . | . | F | 0.60 | 1.08 |
| Leu | 103 | A | A | . | . | . | . | . | 0.18 | . | . | . | 0.30 | 0.34 |
| Ala | 104 | A | A | . | . | . | . | . | 0.14 | . | . | . | −0.60 | 0.20 |
| His | 105 | . | A | B | . | . | . | . | −0.16 | * | . | . | −0.60 | 0.19 |
| Cys | 106 | . | A | B | . | . | . | . | −0.19 | * | . | . | −0.60 | 0.31 |
| Phe | 107 | . | A | B | . | . | . | . | −0.50 | * | . | . | −0.60 | 0.30 |
| Tyr | 108 | . | . | B | . | . | T | . | −0.54 | . | . | . | −0.20 | 0.32 |
| Ser | 109 | . | . | . | . | T | T | . | 0.04 | . | * | F | 0.35 | 0.44 |
| Gly | 110 | . | . | . | . | T | T | . | −0.27 | . | * | F | 0.35 | 0.82 |
| Thr | 111 | . | . | . | . | T | T | . | 0.40 | . | * | F | 0.59 | 0.52 |
| Val | 112 | . | . | B | B | . | . | . | 0.89 | . | * | F | 0.93 | 0.65 |
| Asn | 113 | . | . | . | B | T | . | . | 0.83 | . | * | F | 1.72 | 1.01 |
| Gly | 114 | . | . | . | B | . | . | C | 0.83 | . | * | F | 1.61 | 0.94 |
| Asp | 115 | . | . | . | . | . | T | C | 0.59 | . | * | F | 2.40 | 1.69 |
| Pro | 116 | . | . | . | . | . | T | C | 0.31 | . | * | F | 2.16 | 1.06 |
| Ser | 117 | . | . | . | . | . | T | C | 0.58 | . | * | F | 1.92 | 1.08 |
| Ser | 118 | A | . | . | . | . | T | . | −0.23 | . | . | F | 1.33 | 0.66 |
| Ala | 119 | A | A | . | . | . | . | . | −0.19 | . | . | . | −0.06 | 0.35 |
| Ala | 120 | A | A | . | . | . | . | . | −1.00 | . | . | . | −0.30 | 0.35 |
| Ala | 121 | A | A | . | . | . | . | . | −1.46 | . | . | . | −0.60 | 0.22 |
| Leu | 122 | A | A | . | . | . | . | . | −1.16 | . | . | . | −0.60 | 0.11 |
| Ser | 123 | A | A | . | . | . | . | . | −1.20 | . | . | . | −0.30 | 0.20 |
| Leu | 124 | A | A | . | . | . | . | . | −1.47 | * | * | . | −0.30 | 0.19 |
| Cys | 125 | . | A | B | . | . | . | . | −0.77 | * | * | . | −0.30 | 0.17 |
| Glu | 126 | . | A | B | . | . | . | . | −0.52 | * | * | . | 0.30 | 0.25 |
| Gly | 127 | A | . | . | . | . | . | . | −0.30 | * | * | F | 0.65 | 0.30 |
| Val | 128 | A | . | . | . | . | . | | −0.70 | * | * | F | 0.65 | 0.57 |
| Arg | 129 | . | . | B | . | . | . | . | −0.13 | * | * | F | 0.65 | 0.29 |
| Gly | 130 | . | . | B | B | . | . | . | −0.28 | * | * | . | −0.60 | 0.45 |
| Ala | 131 | . | . | B | B | . | . | . | −1.09 | * | * | . | −0.60 | 0.50 |
| Phe | 132 | . | . | B | B | . | . | . | −1.09 | * | * | . | −0.60 | 0.21 |
| Tyr | 133 | . | . | B | B | . | . | . | −0.23 | * | * | . | −0.60 | 0.21 |
| Leu | 134 | . | A | B | B | . | . | . | −0.93 | * | * | . | −0.60 | 0.36 |
| Leu | 135 | . | A | B | B | . | . | . | −0.83 | . | * | . | −0.60 | 0.42 |
| Gly | 136 | A | A | . | B | . | . | . | −0.94 | . | . | . | −0.60 | 0.42 |
| Glu | 137 | A | A | . | . | . | . | . | −1.13 | . | . | . | −0.60 | 0.44 |
| Ala | 138 | A | A | . | B | . | . | . | −0.89 | . | . | . | −0.60 | 0.38 |
| Tyr | 139 | . | . | B | B | . | . | . | −0.29 | . | . | . | −0.60 | 0.66 |
| Phe | 140 | . | . | B | B | . | . | . | −0.29 | . | . | . | −0.60 | 0.59 |
| Ile | 141 | . | . | B | B | . | . | . | −0.16 | . | . | . | −0.60 | 0.48 |
| Gln | 142 | . | . | B | B | . | . | . | −0.74 | . | . | . | −0.60 | 0.48 |
| Pro | 143 | . | . | B | B | . | . | . | −0.74 | . | . | . | −0.60 | 0.55 |
| Leu | 144 | . | A | . | . | . | . | C | −0.80 | * | . | . | −0.40 | 0.80 |
| Pro | 145 | . | A | . | . | . | . | C | −0.10 | * | . | . | −0.10 | 0.62 |
| Ala | 146 | A | A | . | . | . | . | . | 0.90 | * | * | . | 0.30 | 0.69 |
| Ala | 147 | A | A | . | . | . | . | . | 0.09 | * | . | . | 0.75 | 1.64 |
| Ser | 148 | A | A | . | . | . | . | . | −0.29 | * | . | F | 0.75 | 0.88 |
| Glu | 149 | A | A | . | . | . | . | . | 0.21 | * | . | F | 0.45 | 0.88 |
| Arg | 150 | A | A | . | . | . | . | . | −0.17 | * | . | F | 0.60 | 1.25 |
| Leu | 151 | A | A | . | . | . | . | . | −0.17 | * | . | . | 0.30 | 0.94 |
| Ala | 152 | A | A | . | . | . | . | . | 0.21 | * | * | . | 0.30 | 0.55 |
| Thr | 153 | A | A | . | . | . | . | . | 0.17 | * | * | . | 0.04 | 0.43 |
| Ala | 154 | A | A | . | . | . | . | . | 0.17 | . | . | . | 0.08 | 0.52 |
| Ala | 155 | . | . | . | . | | T | C | 0.10 | . | * | F | 2.07 | 0.89 |
| Pro | 156 | . | . | . | . | . | T | C | 0.70 | . | . | F | 2.86 | 1.24 |
| Gly | 157 | . | . | . | . | T | T | . | 1.08 | . | . | F | 3.40 | 1.90 |
| Glu | 158 | . | . | . | . | . | T | C | 0.80 | . | . | F | 2.86 | 2.90 |
| Lys | 159 | . | . | . | . | . | . | C | 1.18 | . | . | F | 2.32 | 1.90 |
| Pro | 160 | . | . | . | . | . | . | C | 0.96 | . | * | F | 1.98 | 2.97 |
| Pro | 161 | . | . | . | . | . | . | C | 1.17 | . | * | F | 1.64 | 1.41 |
| Ala | 162 | A | A | . | . | . | . | . | 0.81 | . | * | F | 0.60 | 1.22 |
| Pro | 163 | A | A | . | . | . | . | . | 0.78 | . | * | . | −0.60 | 0.68 |
| Leu | 164 | A | A | . | . | . | . | . | −0.08 | . | * | . | −0.60 | 0.60 |
| Gln | 165 | A | A | . | . | . | . | . | −0.68 | * | * | . | −0.60 | 0.49 |
| Phe | 166 | . | A | B | . | . | . | . | −0.36 | * | * | . | −0.60 | 0.26 |
| His | 167 | . | A | B | . | . | . | . | 0.34 | * | * | . | −0.26 | 0.62 |
| Leu | 168 | . | A | B | . | . | . | . | 0.56 | * | * | . | 0.38 | 0.70 |
| Leu | 169 | . | A | B | . | . | . | . | 1.48 | * | * | . | 0.87 | 1.31 |
| Arg | 170 | . | . | . | . | T | T | . | 1.48 | * | . | F | 3.06 | 1.88 |
| Arg | 171 | . | . | . | . | T | T | . | 1.83 | * | . | F | 3.40 | 3.96 |
| Asn | 172 | . | . | . | . | T | T | . | 1.87 | * | . | F | 3.06 | 4.75 |
| Arg | 173 | . | . | . | | T | T | . | 1.82 | * | . | F | 2.72 | 4.05 |
| Gin | 174 | . | . | . | . | T | . | . | 2.29 | . | . | F | 2.43 | 1.53 |
| Gly | 175 | . | . | . | . | T | . | . | 1.83 | . | . | F | 2.19 | 0.94 |
| Asp | 176 | . | . | . | . | T | T | . | 1.41 | . | * | F | 2.30 | 0.48 |
| Val | 177 | . | . | B | . | . | T | | 0.74 | * | . | F | 1.85 | 0.40 |
| Gly | 178 | . | . | . | . | T | T | . | 0.29 | * | . | F | 2.50 | 0.22 |
| Gly | 179 | . | . | B | . | . | T | . | −0.57 | . | * | F | 1.85 | 0.13 |
| Thr | 180 | . | . | B | B | . | . | . | −1.08 | . | * | F | 0.30 | 0.13 |
| Cys | 181 | . | . | B | B | . | . | . | −1.08 | . | . | . | −0.10 | 0.10 |
| Gly | 182 | . | . | B | B | . | . | . | −0.22 | . | . | . | −0.05 | 0.16 |
| Val | 183 | . | . | B | B | . | . | . | 0.12 | . | . | . | 0.30 | 0.19 |
| Val | 184 | . | . | B | B | . | . | . | 0.26 | * | * | . | 0.90 | 0.60 |
| Asp | 185 | . | . | B | . | . | T | . | 0.68 | * | * | F | 1.75 | 0.94 |
| Asp | 186 | . | . | B | . | . | T | . | 1.13 | * | * | F | 2.20 | 2.49 |
| Glu | 187 | . | . | B | . | . | T | . | 1.17 | * | * | F | 2.50 | 5.18 |
| Pro | 188 | . | . | . | . | . | T | C | 1.68 | * | * | F | 3.00 | 4.48 |
| Arg | 189 | . | . | . | . | . | T | C | 2.58 | * | * | F | 2.70 | 2.66 |
| Pro | 190 | . | . | . | . | . | T | C | 1.99 | * | * | F | 2.40 | 3.07 |
| Thr | 191 | . | . | . | . | . | T | C | 1.99 | * | * | F | 2.10 | 2.00 |
| Gly | 192 | . | . | . | . | . | T | C | 1.68 | * | * | F | 1.80 | 1.77 |
| Lys | 193 | A | A | . | . | . | . | . | 1.89 | * | * | F | 0.90 | 1.65 |
| Ala | 194 | A | A | . | . | . | . | . | 1.78 | * | * | F | 0.90 | 1.98 |
| Glu | 195 | A | A | . | . | . | . | . | 1.99 | . | * | F | 0.90 | 3.35 |
| Thr | 196 | A | A | . | . | . | . | . | 2.30 | . | * | F | 0.90 | 2.90 |
| Glu | 197 | A | A | . | . | . | . | . | 2.64 | . | * | F | 0.90 | 4.79 |
| Asp | 198 | A | A | . | . | . | . | . | 2.26 | . | * | F | 0.90 | 4.79 |
| Glu | 199 | A | A | . | . | . | . | . | 2.53 | . | . | F | 0.90 | 3.29 |
| Asp | 200 | A | . | . | . | . | T | . | 2.53 | . | . | F | 1.30 | 2.74 |
| Glu | 201 | A | . | . | . | . | T | . | 2.50 | . | . | F | 1.30 | 2.84 |
| Gly | 202 | A | . | . | . | . | T | . | 2.50 | . | . | F | 1.30 | 1.62 |
| Thr | 203 | A | . | . | . | . | T | . | 2.50 | . | . | F | 1.30 | 1.68 |
| Glu | 204 | A | A | . | . | . | . | . | 2.50 | * | . | F | 0.90 | 1.62 |
| Gly | 205 | A | A | . | . | . | . | . | 2.16 | * | . | F | 1.20 | 2.84 |
| Glu | 206 | A | A | . | . | . | . | . | 1.94 | * | . | F | 1.50 | 1.95 |
| Asp | 207 | . | A | . | . | T | . | . | 2.29 | * | . | F | 2.20 | 1.74 |
| Glu | 208 | . | A | . | . | . | . | C | 2.31 | * | . | F | 2.30 | 3.04 |
| Gly | 209 | . | . | . | . | . | T | C | 2.01 | * | . | F | 3.00 | 1.85 |
| Pro | 210 | . | . | . | . | T | T | . | 2.14 | . | . | F | 2.60 | 1.48 |
| Gln | 211 | . | . | . | . | T | T | . | 2.14 | . | . | F | 2.30 | 1.32 |
| Trp | 212 | . | . | . | . | . | T | C | 2.14 | . | . | F | 1.44 | 2.32 |
| Ser | 213 | . | . | . | . | . | . | C | 1.93 | . | . | F | 1.78 | 2.50 |
| Pro | 214 | . | . | . | . | T | T | . | 1.69 | . | . | F | 2.12 | 2.23 |
| Gln | 215 | . | . | . | . | . | T | C | 1.09 | . | . | F | 1.56 | 2.15 |
| Asp | 216 | . | . | . | . | . | T | C | 1.09 | . | * | F | 2.40 | 1.32 |
| Pro | 217 | . | . | . | . | . | T | C | 1.03 | . | . | F | 2.16 | 1.48 |
| Ala | 218 | . | . | . | . | T | . | . | 0.48 | . | . | F | 1.77 | 0.85 |
| Leu | 219 | . | . | B | . | . | . | . | 0.34 | * | . | F | 0.53 | 0.38 |
| Gln | 220 | . | . | B | . | . | . | . | 0.34 | * | . | F | −0.01 | 0.24 |
| Gly | 221 | . | . | B | . | . | T | . | 0.13 | * | * | F | −0.05 | 0.41 |
| Val | 222 | . | . | B | . | . | T | . | 0.03 | * | . | F | −0.05 | 0.77 |
| Gly | 223 | . | . | B | . | . | T | . | 0.28 | * | . | F | 0.25 | 0.64 |
| Gln | 224 | . | . | B | . | . | T | . | 0.78 | * | * | F | 0.25 | 0.64 |
| Pro | 225 | . | . | B | . | . | . | . | 0.43 | . | . | F | 0.20 | 1.25 |
| Thr | 226 | . | . | . | . | T | . | . | 0.48 | . | * | F | 0.60 | 1.25 |
| Gly | 227 | . | . | . | . | . | T | C | 0.44 | * | * | F | 0.45 | 0.97 |
| Thr | 228 | . | . | B | . | . | T | . | 0.90 | * | * | F | 0.25 | 0.44 |
| Gly | 229 | . | . | B | . | . | T | . | 0.94 | . | * | F | 0.85 | 0.60 |
| Ser | 230 | . | . | B | . | . | T | . | 1.20 | . | * | F | 1.30 | 1.20 |
| Ile | 231 | . | A | B | . | . | . | . | 1.62 | . | * | F | 0.90 | 1.67 |
| Arg | 232 | . | A | B | . | . | . | . | 1.27 | . | * | F | 0.90 | 3.30 |
| Lys | 233 | . | A | B | . | . | . | . | 0.72 | . | . | F | 0.90 | 2.13 |
| Lys | 234 | . | A | B | B | . | . | . | 0.77 | . | . | F | 0.90 | 2.26 |
| Arg | 235 | . | A | B | B | . | . | . | 0.77 | . | . | F | 0.90 | 1.55 |
| Phe | 236 | . | . | B | B | . | . | . | 1.62 | . | * | . | 0.75 | 1.04 |
| Val | 237 | . | . | B | B | . | . | . | 1.62 | . | * | . | 0.30 | 0.71 |
| Ser | 238 | . | . | B | . | . | T | . | 1.33 | * | * | . | 0.70 | 0.71 |
| Ser | 239 | . | . | . | . | . | T | C | 0.43 | * | . | . | 0.15 | 1.28 |
| His | 240 | . | . | . | . | . | T | C | 0.32 | * | * | . | 0.45 | 1.28 |
| Arg | 241 | . | . | . | . | . | T | C | 0.71 | * | . | . | 1.05 | 1.65 |
| Tyr | 242 | A | . | . | B | . | . | . | 0.97 | * | . | . | 0.45 | 1.78 |
| Val | 243 | A | . | . | B | . | . | . | 0.46 | * | . | . | 0.45 | 1.29 |
| Glu | 244 | . | . | B | B | . | . | . | −0.10 | * | . | . | −0.30 | 0.54 |
| Thr | 245 | . | . | B | B | . | . | . | −0.66 | * | . | . | −0.60 | 0.26 |
| Met | 246 | A | . | B | B | . | . | . | −0.77 | * | . | . | −0.60 | 0.35 |
| Leu | 247 | A | . | . | B | . | . | . | −0.52 | . | . | . | 0.30 | 0.34 |
| Val | 248 | A | . | . | B | . | . | . | 0.03 | . | . | . | −0.30 | 0.41 |
| Ala | 249 | A | . | . | B | . | . | . | −0.57 | . | . | . | −0.30 | 0.55 |
| Asp | 250 | A | . | . | . | . | T | . | −0.84 | . | . | F | 0.25 | 0.66 |
| Gln | 251 | A | . | . | . | . | T | . | −0.24 | . | . | F | 0.25 | 0.90 |
| Ser | 252 | A | . | . | . | . | T | . | −0.13 | . | . | F | 1.30 | 1.54 |
| Met | 253 | A | . | . | . | . | T | . | 0.69 | . | * | . | 0.70 | 0.80 |
| Ala | 254 | A | . | . | . | . | . | . | 0.93 | . | * | . | −0.10 | 0.63 |
| Glu | 255 | A | . | . | . | . | . | . | 0.63 | . | * | . | −0.10 | 0.46 |
| Phe | 256 | A | . | . | . | . | . | . | 0.29 | . | * | . | −0.10 | 0.63 |
| His | 257 | A | . | . | . | . | T | . | −0.22 | * | . | . | 0.10 | 0.61 |
| Gly | 258 | A | . | . | . | . | T | . | 0.42 | * | . | F | 0.25 | 0.29 |
| Ser | 259 | A | . | . | . | . | T | . | 0.98 | * | * | F | 0.25 | 0.68 |
| Gly | 260 | A | . | . | . | . | T | . | 0.73 | * | * | F | 0.85 | 0.68 |
| Leu | 261 | A | A | . | . | . | . | . | 0.62 | . | . | F | 0.00 | 1.07 |
| Lys | 262 | A | A | . | . | . | . | . | −0.16 | . | . | . | −0.60 | 0.66 |
| His | 263 | . | A | B | . | . | . | . | −0.12 | * | . | . | −0.60 | 0.55 |
| Tyr | 264 | . | A | B | . | . | . | . | −0.63 | * | . | . | −0.60 | 0.96 |
| Leu | 265 | . | A | B | . | . | . | . | −0.99 | * | . | . | −0.60 | 0.40 |
| Leu | 266 | . | A | B | . | . | . | . | −0.48 | * | . | . | −0.60 | 0.25 |
| Thr | 267 | . | A | B | . | . | . | . | −1.38 | * | . | . | −0.60 | 0.22 |
| Leu | 268 | . | A | B | . | . | . | . | −1.93 | * | . | . | −0.60 | 0.19 |
| Phe | 269 | A | A | . | . | . | . | . | −2.28 | * | * | . | −0.60 | 0.24 |
| Ser | 270 | A | A | . | . | . | . | . | −1.36 | * | * | . | −0.60 | 0.17 |
| Val | 271 | A | A | . | . | . | . | . | −1.36 | * | * | . | −0.60 | 0.39 |
| Ala | 272 | A | A | . | . | . | . | . | −1.29 | * | * | . | −0.60 | 0.38 |
| Ala | 273 | A | A | . | . | . | . | . | −0.43 | * | * | . | −0.60 | 0.44 |
| Arg | 274 | A | A | . | . | . | . | . | 0.23 | * | * | . | −0.15 | 1.18 |
| Leu | 275 | A | A | . | . | . | . | . | 0.32 | * | * | . | 0.45 | 1.59 |
| Tyr | 276 | . | . | . | . | T | . | . | 0.88 | * | * | . | 1.39 | 2.44 |
| Lys | 277 | . | . | B | . | . | . | . | 0.58 | * | * | F | 1.48 | 1.67 |
| His | 278 | . | . | B | . | . | T | . | 1.28 | . | * | F | 1.12 | 1.42 |
| Pro | 279 | . | . | B | . | . | T | . | 1.17 | . | * | F | 2.36 | 1.77 |
| Ser | 280 | . | . | . | . | T | T | . | 1.68 | . | * | F | 3.40 | 1.43 |
| Ile | 281 | . | . | B | . | . | T | . | 1.07 | . | * | F | 2.36 | 1.41 |
| Arg | 282 | . | . | B | B | . | . | . | 0.72 | . | * | F | 1.47 | 0.67 |
| Asn | 283 | . | . | B | B | . | . | . | −0.06 | * | * | F | 1.13 | 0.67 |
| Ser | 284 | . | . | B | B | . | . | . | −0.70 | * | * | F | 0.19 | 0.79 |
| Val | 285 | . | . | B | B | . | . | . | −1.26 | * | * | . | −0.30 | 0.30 |
| Ser | 286 | . | . | B | B | . | . | . | −1.22 | . | * | . | −0.60 | 0.14 |
| Leu | 287 | . | . | B | B | . | . | . | −1.29 | . | * | . | −0.60 | 0.08 |
| Val | 288 | . | . | B | B | . | . | . | −2.18 | * | . | . | −0.60 | 0.21 |
| Val | 289 | . | . | B | B | . | . | . | −2.69 | . | * | . | −0.60 | 0.11 |
| Val | 290 | . | . | B | B | . | . | . | −2.69 | . | . | . | −0.60 | 0.11 |
| Lys | 291 | . | . | B | B | . | . | . | −3.28 | . | . | . | −0.60 | 0.11 |
| Ile | 292 | . | . | B | B | . | . | . | −2.50 | . | . | . | −0.60 | 0.10 |
| Leu | 293 | . | . | B | B | . | . | . | −1.64 | . | * | . | −0.60 | 0.19 |
| Val | 294 | . | . | B | B | . | . | . | −0.79 | . | . | . | −0.30 | 0.16 |
| Ile | 295 | . | . | B | B | . | . | . | 0.07 | . | * | . | 0.00 | 0.39 |
| His | 296 | A | . | . | B | . | . | . | 0.07 | . | * | . | 0.90 | 0.81 |
| Asp | 297 | A | . | . | B | . | . | . | 0.61 | . | . | F | 1.80 | 2.19 |
| Glu | 298 | A | . | . | . | . | . | . | 1.21 | * | . | F | 2.30 | 3.09 |
| Gln | 299 | . | . | . | . | T | . | . | 2.07 | * | . | F | 3.00 | 3.51 |
| Lys | 300 | . | . | . | . | . | . | C | 2.10 | . | . | F | 2.50 | 3.64 |
| Gly | 301 | . | . | . | . | . | T | C | 1.82 | . | . | F | 2.40 | 1.56 |
| Pro | 302 | . | . | . | . | . | T | C | 1.52 | . | . | F | 2.10 | 1.30 |
| Glu | 303 | . | . | B | . | . | T | . | 1.52 | * | . | F | 1.45 | 0.87 |
| Val | 304 | A | . | . | . | . | T | . | 0.93 | * | . | F | 1.00 | 1.42 |
| Thr | 305 | A | . | . | . | . | T | . | 0.30 | . | * | F | 0.85 | 0.93 |
| Ser | 306 | A | . | . | . | . | T | . | −0.17 | . | * | F | 0.85 | 0.54 |
| Asn | 307 | A | . | . | . | . | T | . | −0.27 | . | * | F | −0.05 | 0.60 |
| Ala | 308 | A | . | . | . | . | T | . | −1.08 | * | * | . | −0.20 | 0.60 |
| Ala | 309 | A | . | . | . | . | . | . | −0.11 | * | * | . | −0.40 | 0.37 |
| Leu | 310 | A | . | . | . | . | . | . | 0.20 | * | * | . | −0.10 | 0.45 |
| Thr | 311 | . | . | B | . | . | . | . | −0.20 | * | * | . | −0.10 | 0.72 |
| Leu | 312 | . | . | B | . | . | . | . | −0.87 | * | * | . | −0.40 | 0.61 |
| Arg | 313 | . | . | B | . | . | . | . | −0.28 | * | * | . | −0.40 | 0.40 |
| Asn | 314 | . | . | . | . | T | . | . | 0.02 | * | * | . | 0.30 | 0.44 |
| Phe | 315 | . | . | . | . | T | T | . | 0.83 | * | * | . | 0.20 | 0.57 |
| Cys | 316 | . | . | . | . | T | T | . | 1.19 | * | * | . | 0.20 | 0.50 |
| Asn | 317 | . | . | . | . | T | T | . | 2.00 | * | * | . | 0.20 | 0.62 |
| Trp | 318 | . | . | . | . | T | T | . | 1.86 | * | . | . | 0.35 | 1.25 |
| Gln | 319 | . | . | . | . | T | . | . | 1.86 | . | . | . | 0.45 | 3.16 |
| Lys | 320 | . | . | . | . | T | . | . | 2.34 | * | . | F | 0.60 | 3.16 |
| Gln | 321 | . | . | . | . | T | . | . | 2.80 | . | . | F | 0.94 | 4.65 |
| His | 322 | . | . | . | . | . | . | C | 2.50 | * | . | F | 1.68 | 4.15 |
| Asn | 323 | . | . | . | . | . | . | C | 2.79 | * | . | F | 2.02 | 2.78 |
| Pro | 324 | . | . | . | . | . | T | C | 2.90 | . | . | F | 2.56 | 2.68 |
| Pro | 325 | . | . | . | . | T | T | . | 2.86 | * | . | F | 3.40 | 3.86 |
| Ser | 326 | . | . | . | . | . | T | C | 2.27 | . | . | F | 2.86 | 4.01 |
| Asp | 327 | . | . | . | . | . | T | C | 2.30 | . | . | F | 2.52 | 2.62 |
| Arg | 328 | A | A | . | . | . | . | . | 2.27 | . | . | F | 1.58 | 2.94 |
| Asp | 329 | A | A | . | . | . | . | . | 2.23 | * | . | F | 1.24 | 2.98 |
| Ala | 330 | A | A | . | . | . | . | . | 2.44 | * | . | . | 0.90 | 2.80 |
| Glu | 331 | A | A | . | . | . | . | . | 2.43 | * | . | . | 0.75 | 2.38 |
| His | 332 | A | . | . | . | . | T | . | 1.84 | * | . | . | 1.15 | 2.06 |
| Tyr | 333 | A | . | . | . | . | T | . | 0.84 | * | . | . | 0.85 | 2.06 |
| Asp | 334 | A | . | . | . | . | T | . | 0.03 | . | . | . | 0.70 | 0.83 |
| Thr | 335 | A | . | . | . | . | T | . | −0.08 | . | . | . | −0.20 | 0.51 |
| Ala | 336 | A | A | . | . | . | . | . | −0.39 | * | . | . | −0.60 | 0.28 |
| Ile | 337 | A | A | . | . | . | . | . | −0.24 | * | . | . | −0.60 | 0.24 |
| Leu | 338 | . | A | B | . | . | . | . | 0.00 | . | . | . | −0.60 | 0.33 |
| Phe | 339 | . | A | B | . | . | . | . | 0.00 | . | * | . | −0.60 | 0.56 |
| Thr | 340 | . | A | B | . | . | . | . | −0.50 | . | . | F | 0.00 | 1.34 |
| Arg | 341 | . | A | B | . | . | . | . | −0.58 | . | * | F | 0.25 | 1.34 |
| Gln | 342 | . | A | . | . | T | . | . | −0.03. | . | * | F | 1.35 | 0.83 |
| Asp | 343 | . | A | . | . | T | . | . | 0.48 | . | * | F | 1.60 | 0.57 |
| Leu | 344 | . | A | . | . | T | . | . | 1.18 | * | . | F | 2.15 | 0.39 |
| Cys | 345 | . | . | . | . | T | T | . | 1.18 | . | * | F | 2.50 | 0.39 |
| Gly | 346 | . | . | . | . | T | T | . | 0.40 | . | * | F | 2.25 | 0.34 |
| Ser | 347 | . | . | . | . | T | T | . | 0.40 | . | . | F | 1.10 | 0.22 |
| Gln | 348 | . | . | B | . | . | T | . | 0.09 | . | . | F | 1.35 | 0.68 |
| Thr | 349 | . | . | B | . | . | . | . | 0.09 | . | . | F | 0.90 | 0.99 |
| Cys | 350 | . | . | B | . | . | . | . | 0.41 | . | . | F | 0.05 | 0.61 |
| Asp | 351 | . | . | B | . | . | T | . | 0.16 | * | . | F | 0.25 | 0.35 |
| Thr | 352 | . | . | B | . | . | T | . | −0.13 | . | . | F | 0.25 | 0.24 |
| Leu | 353 | . | . | B | . | . | T | . | −0.13 | . | . | . | 0.10 | 0.45 |
| Gly | 354 | . | . | B | . | . | T | . | −0.68 | . | . | . | 0.70 | 0.45 |
| Met | 355 | . | . | B | . | . | . | . | −0.36 | . | . | . | −0.10 | 0.23 |
| Ala | 356 | . | . | B | . | . | . | . | −0.67 | . | . | . | −0.10 | 0.28 |
| Asp | 357 | . | . | B | . | . | T | . | −1.21 | . | . | . | 0.10 | 0.41 |
| Val | 358 | . | . | B | . | . | T | . | −1.07 | . | . | . | 0.10 | 0.30 |
| Gly | 359 | . | . | B | . | . | T | . | −0.72 | . | . | . | 0.10 | 0.16 |
| Thr | 360 | . | . | B | . | . | T | | −0.33 | . | . | . | 0.70 | 0.16 |
| Val | 361 | . | . | B | . | . | . | . | −0.04 | . | * | . | 0.24 | 0.34 |
| Cys | 362 | . | . | B | . | . | . | . | 0.07 | * | . | . | 1.18 | 0.46 |
| Asp | 363 | . | . | B | . | . | T | . | 0.62 | * | . | F | 1.87 | 0.62 |
| Pro | 364 | . | . | . | . | T | T | . | 0.30 | * | . | F | 3.06 | 1.12 |
| Ser | 365 | . | . | . | . | T | T | . | 0.31 | * | . | F | 3.40 | 1.12 |
| Arg | 366 | . | . | . | . | T | T | . | 0.31 | * | . | F | 2.91 | 0.90 |
| Ser | 367 | . | . | . | B | T | . | . | 0.09 | * | . | F | 1.87 | 0.43 |
| Cys | 368 | . | . | B | B | . | . | . | 0.09 | * | . | . | 0.38 | 0.22 |
| Ser | 369 | . | . | B | B | . | . | . | 0.30 | * | . | . | 0.64 | 0.20 |
| Val | 370 | . | . | B | B | . | . | . | 0.60 | * | . | . | 0.30 | 0.25 |
| Ile | 371 | . | . | B | B | . | . | . | 0.14 | * | . | . | 0.60 | 0.77 |
| Glu | 372 | . | . | B | B | . | . | . | −0.37 | . | . | . | 0.60 | 0.57 |
| Asp | 373 | A | . | . | . | . | T | . | 0.30 | . | . | F | 1.15 | 0.63 |
| Asp | 374 | A | . | . | . | . | T | . | 0.01 | * | . | . | 1.30 | 1.56 |
| Gly | 375 | A | . | . | . | . | T | . | 0.28 | . | . | . | 1.00 | 0.91 |
| Leu | 376 | A | . | . | . | . | T | . | 0.47 | * | . | . | 0.70 | 0.55 |
| Gln | 377 | A | A | . | . | . | . | . | 0.16 | . | . | . | −0.30 | 0.29 |
| Ala | 378 | A | A | . | . | . | . | . | −0.16 | * | . | . | −0.60 | 0.42 |
| Ala | 379 | A | A | . | . | . | . | . | −0.74 | * | . | . | −0.60 | 0.73 |
| Phe | 380 | A | A | . | . | . | . | . | −0.43 | * | . | . | −0.60 | 0.43 |
| Thr | 381 | A | A | . | . | . | . | . | 0.38 | * | * | . | −0.60 | 0.57 |
| Thr | 382 | A | A | . | . | . | . | . | −0.43 | * | . | . | −0.30 | 0.98 |
| Ala | 383 | A | A | . | . | . | . | . | −0.19 | * | . | . | −0.60 | 0.94 |
| His | 384 | A | A | . | . | . | . | . | 0.37 | * | . | . | −0.30 | 0.64 |
| Glu | 385 | A | A | . | . | . | . | . | 0.21 | * | . | . | −0.30 | 0.61 |
| Leu | 386 | A | A | . | . | . | . | . | −0.18 | * | . | . | −0.30 | 0.45 |
| Gly | 387 | A | . | . | B | . | . | . | 0.13 | * | . | . | −0.60 | 0.28 |
| His | 388 | A | . | . | B | . | . | . | 0.12 | * | . | . | −0.60 | 0.26 |
| Val | 389 | A | . | . | B | . | . | . | −0.06 | * | . | . | −0.60 | 0.32 |
| Phe | 390 | A | . | . | B | . | . | . | −0.09 | * | . | . | −0.60 | 0.49 |
| Asn | 391 | . | . | B | B | . | . | . | 0.72 | * | . | . | −0.60 | 0.49 |
| Met | 392 | . | . | B | . | . | T | . | 1.07 | * | . | . | 0.25 | 1.11 |
| Pro | 393 | A | . | . | . | . | T | . | 0.51 | * | . | . | 0.85 | 2.14 |
| His | 394 | . | . | . | . | T | T | . | 1.41 | * | . | F | 1.70 | 1.34 |
| Asp | 395 | A | . | . | . | . | T | . | 2.11 | * | . | F | 1.30 | 2.72 |
| Asp | 396 | A | A | . | . | . | . | . | 1.44 | * | . | F | 0.90 | 3.04 |
| Ala | 397 | A | A | . | . | . | . | . | 1.46 | * | . | F | 0.90 | 1.20 |
| Lys | 398 | A | A | . | . | . | . | . | 1.37 | * | * | F | 0.75 | 0.73 |
| Gln | 399 | A | A | . | . | . | . | . | 0.59 | . | * | . | 0.60 | 0.58 |
| Cys | 400 | . | A | B | . | . | . | . | 0.59 | . | * | . | −0.30 | 0.48 |
| Ala | 401 | . | A | B | . | . | . | . | 0.24 | . | * | . | 0.30 | 0.38 |
| Ser | 402 | . | . | B | . | . | T | . | −0.02 | . | * | . | 0.10 | 0.22 |
| Leu | 403 | . | . | B | . | . | T | . | −0.07 | . | . | . | 0.04 | 0.30 |
| Asn | 404 | . | . | . | . | T | T | . | −0.07 | . | . | . | 0.68 | 0.48 |
| Gly | 405 | . | . | . | . | T | T | . | 0.60 | . | . | F | 1.37 | 0.62 |
| Val | 406 | . | . | . | . | . | . | C | 0.89 | . | . | F | 1.96 | 1.26 |
| Asn | 407 | . | . | . | . | . | T | C | 1.16 | . | . | F | 2.40 | 1.05 |
| Gln | 408 | A | . | . | . | . | T | . | 1.37 | * | . | F | 1.96 | 1.44 |
| Asp | 409 | A | . | . | . | . | T | . | 0.77 | * | . | F | 1.72 | 1.92 |
| Ser | 410 | A | . | . | . | . | T | . | 0.52 | . | . | . | 1.33 | 1.18 |
| His | 411 | A | A | . | . | . | . | . | 1.08 | . | * | . | −0.06 | 0.69 |
| Met | 412 | A | A | . | . | . | . | . | 0.48 | . | . | . | 0.30 | 0.55 |
| Met | 413 | A | A | . | . | . | . | . | −0.33 | . | . | . | −0.60 | 0.41 |
| Ala | 414 | A | A | . | . | . | . | . | −0.63 | . | . | . | −0.60 | 0.25 |
| Ser | 415 | A | A | . | . | . | . | . | −0.33 | * | . | . | −0.60 | 0.34 |
| Met | 416 | A | A | . | . | . | . | . | −1.11 | * | * | . | −0.60 | 0.55 |
| Leu | 417 | A | . | . | . | . | T | . | −0.51 | * | . | . | −0.20 | 0.45 |
| Ser | 418 | A | . | . | . | . | T | . | 0.06 | * | . | . | 0.38 | 0.56 |
| Asn | 419 | A | . | . | . | . | T | . | 0.34 | . | . | . | 0.66 | 0.76 |
| Leu | 420 | . | . | . | . | . | T | C | 0.64 | . | . | . | 1.29 | 1.24 |
| Asp | 421 | . | . | . | . | T | T | . | 1.03 | . | . | . | 2.37 | 1.60 |
| His | 422 | . | . | . | . | T | T | . | 1.56 | . | . | F | 2.80 | 1.54 |
| Ser | 423 | . | . | . | . | . | T | C | 1.56 | . | . | F | 1.72 | 1.97 |
| Gln | 424 | . | . | . | . | . | T | C | 1.34 | . | . | F | 1.44 | 1.58 |
| Pro | 425 | . | . | . | . | T | . | . | 1.49 | . | . | F | 0.86 | 1.79 |
| Trp | 426 | . | . | . | . | T | . | . | 1.19 | . | . | F | 0.43 | 0.72 |
| Ser | 427 | . | . | . | . | . | T | C | 0.63 | . | . | F | 0.15 | 0.55 |
| Pro | 428 | . | . | . | . | T | T | . | 0.69 | . | . | F | 0.35 | 0.36 |
| Cys | 429 | . | . | . | . | T | T | . | 0.09 | . | . | . | 0.20 | 0.54 |
| Ser | 430 | . | . | B | . | . | T | . | −0.59 | . | . | . | −0.20 | 0.40 |
| Ala | 431 | . | . | B | B | . | . | . | −0.61 | . | . | . | −0.60 | 0.18 |
| Tyr | 432 | . | . | B | B | . | . | . | −0.61 | . | . | . | −0.60 | 0.49 |
| Met | 433 | . | . | B | B | . | . | . | −1.10 | . | . | . | −0.60 | 0.49 |
| Ile | 434 | . | . | B | B | . | . | . | −1.24 | * | . | . | −0.60 | 0.42 |
| Thr | 435 | . | . | B | B | . | . | . | −0.94 | * | . | . | −0.60 | 0.22 |
| Ser | 436 | . | . | B | B | . | . | . | −0.36 | * | . | . | −0.60 | 0.37 |
| Phe | 437 | . | . | B | B | . | . | . | −0.46 | * | . | . | −0.60 | 0.85 |
| Leu | 438 | . | . | B | . | . | T | . | 0.11 | * | . | F | 0.56 | 0.58 |
| Asp | 439 | . | . | . | . | T | T | . | 0.66 | * | . | F | 1.27 | 0.59 |
| Asn | 440 | . | . | . | . | . | T | C | 0.97 | . | . | F | 1.38 | 0.68 |
| Gly | 441 | . | . | . | . | T | T | . | 0.60 | . | . | F | 2.94 | 1.42 |
| His | 442 | . | . | . | . | T | T | . | 0.49 | . | . | F | 3.10 | 0.46 |
| Gly | 443 | A | . | . | . | . | T | . | 0.70 | . | . | F | 1.49 | 0.23 |
| Glu | 444 | A | . | . | . | . | T | . | 0.70 | . | . | . | 1.03 | 0.23 |
| Cys | 445 | . | . | B | . | . | T | . | 0.74 | . | * | . | 1.32 | 0.29 |
| Leu | 446 | . | A | B | . | . | . | . | 0.88 | . | . | . | 1.25 | 0.58 |
| Met | 447 | . | A | B | . | . | . | . | 0.91 | * | . | . | 1.28 | 0.52 |
| Asp | 448 | . | A | . | . | T | . | . | 1.26 | * | . | F | 2.02 | 1.67 |
| Lys | 449 | . | A | . | . | . | . | C | 1.04 | * | . | F | 2.16 | 3.26 |
| Pro | 450 | . | . | . | . | T | T | . | 0.82 | * | * | F | 3.40 | 5.10 |
| Gln | 451 | . | . | . | . | T | T | . | 1.63 | * | * | F | 3.06 | 2.14 |
| Asn | 452 | . | . | B | . | . | T | . | 1.42 | * | * | F | 2.02 | 1.85 |
| Pro | 453 | . | . | B | . | . | T | . | 1.21 | * | * | F | 0.63 | 0.99 |
| Ile | 454 | . | . | B | . | . | . | . | 0.82 | * | * | F | 0.09 | 0.88 |
| Gln | 455 | . | . | B | . | . | . | . | 1.03 | * | * | F | −0.25 | 0.54 |
| Leu | 456 | . | . | B | . | . | T | . | 0.22 | * | * | F | 0.25 | 0.59 |
| Pro | 457 | . | . | B | . | . | T | . | 0.01 | * | * | F | 0.25 | 0.69 |
| Gly | 458 | . | . | B | . | . | T | . | −0.12 | . | * | F | 0.25 | 0.62 |
| Asp | 459 | . | . | B | . | . | T | . | 0.46 | . | * | F | 0.25 | 0.74 |
| Leu | 460 | . | . | . | . | . | T | C | 0.16 | . | * | F | 1.05 | 0.96 |
| Pro | 461 | . | . | B | . | . | T | . | 0.72 | . | * | F | 0.85 | 0.93 |
| Gly | 462 | . | . | B | . | . | T | . | 0.93 | . | . | F | 0.25 | 0.88 |
| Thr | 463 | . | . | B | . | . | T | . | 0.69 | . | * | F | 0.74 | 1.78 |
| Ser | 464 | . | . | B | . | . | . | . | 0.69 | * | . | F | 1.48 | 1.16 |
| Tyr | 465 | . | . | . | . | T | . | . | 1.61 | * | . | F | 2.22 | 1.88 |
| Asp | 466 | . | . | . | . | T | T | . | 1.82 | . | . | . | 2.61 | 2.56 |
| Ala | 467 | . | . | . | . | T | T | . | 1.50 | * | . | F | 3.40 | 3.31 |
| Asn | 468 | . | . | . | . | T | T | . | 1.81 | . | * | F | 2.76 | 1.13 |
| Arg | 469 | . | . | B | . | . | T | . | 1.41 | . | * | F | 2.32 | 1.17 |
| Gln | 470 | . | . | B | B | . | . | . | 1.34 | * | * | . | 0.53 | 1.01 |
| Cys | 471 | . | . | B | B | . | . | . | 0.64 | * | * | . | 0.04 | 0.09 |
| Gln | 472 | . | . | B | B | . | . | . | 0.89 | . | . | . | −0.60 | 0.40 |
| Phe | 473 | . | . | B | B | . | . | . | 0.89 | . | . | . | −0.26 | 0.23 |
| Thr | 474 | . | . | B | B | . | . | . | 0.78 | . | . | . | 0.08 | 0.74 |
| Phe | 475 | . | . | . | B | T | . | . | 0.48 | . | * | . | 1.72 | 0.71 |
| Gly | 476 | . | . | . | . | T | T | . | 1.19 | . | * | F | 2.76 | 1.10 |
| Glu | 477 | . | . | . | . | T | T | . | 1.16 | . | * | F | 3.40 | 1.52 |
| Asp | 478 | . | . | . | . | T | T | . | 1.19 | * | . | F | 3.06 | 2.39 |
| Ser | 479 | . | . | . | . | T | T | . | 1.29 | * | . | F | 2.72 | 1.30 |
| Lys | 480 | . | . | . | . | T | . | . | 1.99 | * | . | F | 2.43 | 1.16 |
| His | 481 | . | . | . | . | T | . | . | 1.74 | * | . | F | 2.34 | 1.16 |
| Cys | 482 | . | . | . | . | . | T | C | 1.16 | * | . | F | 2.10 | 0.87 |
| Pro | 483 | A | . | . | . | . | T | . | 0.86 | . | . | F | 2.15 | 0.44 |
| Asp | 484 | . | . | . | . | T | T | . | 0.84 | * | . | F | 2.50 | 0.43 |
| Ala | 485 | A | . | . | . | . | T | . | 0.13 | * | . | F | 2.00 | 1.17 |
| Ala | 486 | A | . | . | . | . | . | . | −0.13 | . | . | F | 1.40 | 0.41 |
| Ser | 487 | . | . | B | . | T | T | . | 0.22 | . | . | F | 1.75 | 0.33 |
| Thr | 488 | . | . | B | . | . | T | . | −0.38 | * | . | F | 0.50 | 0.46 |
| Cys | 489 | . | . | B | . | . | T | . | −0.67 | * | . | F | −0.05 | 0.38 |
| Ser | 490 | . | . | B | . | . | T | . | −0.74 | . | . | F | −0.05 | 0.30 |
| Thr | 491 | . | . | B | B | . | . | . | −0.47 | . | . | . | −0.60 | 0.11 |
| Leu | 492 | . | . | B | B | . | . | . | −0.51 | . | . | . | −0.60 | 0.30 |
| Trp | 493 | . | . | B | B | . | . | . | −0.51 | . | . | . | −0.60 | 0.22 |
| Cys | 494 | . | . | B | B | . | . | . | −0.14 | . | . | . | −0.60 | 0.22 |
| Thr | 495 | . | . | B | B | T | . | . | −0.19 | . | . | F | −0.05 | 0.36 |
| Gly | 496 | . | . | . | B | T | . | . | −0.22 | . | . | F | −0.05 | 0.34 |
| Thr | 497 | . | . | . | . | T | T | . | −0.27 | . | . | F | 0.65 | 0.62 |
| Ser | 498 | . | . | . | . | T | T | . | −0.79 | . | . | F | 0.65 | 0.32 |
| Gly | 499 | . | . | . | . | T | T | . | −0.98 | . | . | F | 0.35 | 0.27 |
| Gly | 500 | . | . | . | . | T | T | . | −1.33 | . | . | F | 0.35 | 0.14 |
| Val | 501 | . | . | B | B | . | . | . | −0.99 | . | . | . | −0.60 | 0.05 |
| Leu | 502 | . | . | B | B | . | . | . | −0.99 | . | . | . | −0.60 | 0.10 |
| Val | 503 | . | . | B | B | . | . | . | −0.64 | . | . | . | −0.60 | 0.14 |
| Cys | 504 | . | . | B | . | . | T | . | −0.33 | . | . | . | −0.20 | 0.38 |
| Gln | 505 | . | . | B | . | . | T | . | −0.69 | . | . | . | 0.10 | 0.62 |
| Thr | 506 | . | . | B | . | . | T | . | −0.04 | . | . | F | 0.25 | 0.72 |
| Lys | 507 | . | . | B | . | . | T | . | 0.48 | . | . | F | 0.40 | 2.09 |
| His | 508 | . | . | . | . | . | . | C | 0.74 | . | . | . | −0.05 | 1.27 |
| Phe | 509 | . | . | B | . | . | . | . | 1.41 | . | . | . | −0.40 | 0.89 |
| Pro | 510 | . | . | . | . | T | . | . | 1.07 | . | . | . | 0.30 | 0.74 |
| Trp | 511 | . | . | . | . | T | T | . | 1.07 | . | . | . | 0.20 | 0.54 |
| Ala | 512 | . | . | . | . | T | T | . | 0.72 | . | . | . | 0.51 | 0.90 |
| Asp | 513 | . | . | . | . | T | T | . | 0.09 | . | . | F | 1.27 | 0.78 |
| Gly | 514 | . | . | . | . | T | T | . | 0.44 | . | . | F | 1.58 | 0.40 |
| Thr | 515 | . | . | . | . | T | T | . | 0.66 | . | . | F | 2.49 | 0.39 |
| Ser | 516 | . | . | . | . | T | T | . | 0.60 | . | * | F | 3.10 | 0.40 |
| Cys | 517 | . | . | . | . | T | T | . | 1.23 | . | * | F | 2.49 | 0.40 |
| Gly | 518 | . | . | . | . | T | T | . | 0.94 | . | * | F | 2.48 | 0.56 |
| Glu | 519 | . | . | . | . | T | . | . | 0.62 | . | * | F | 1.67 | 0.44 |
| Gly | 520 | . | . | . | . | T | . | . | 0.04 | . | * | F | 1.36 | 0.44 |
| Lys | 521 | . | . | . | . | T | . | . | 0.34 | . | * | F | 0.45 | 0.31 |
| Trp | 522 | . | . | . | . | T | . | . | 0.67 | . | * | . | 0.90 | 0.29 |
| Cys | 523 | . | . | B | . | . | T | . | 1.06 | . | * | . | −0.20 | 0.29 |
| Ile | 524 | . | . | B | . | . | T | . | 0.39 | . | * | . | 0.70 | 0.29 |
| Asn | 525 | . | . | . | . | T | T | . | −0.12 | . | * | . | 0.20 | 0.15 |
| Gly | 526 | . | . | . | . | T | T | . | −0.17 | * | * | F | 0.65 | 0.20 |
| Lys | 527 | . | . | . | . | T | . | . | 0.17 | * | * | F | 0.45 | 0.47 |
| Cys | 528 | . | . | . | . | T | T | . | 0.52 | . | * | . | 1.40 | 0.58 |
| Val | 529 | . | . | B | . | . | T | . | 1.41 | * | * | . | 1.04 | 0.85 |
| Asn | 530 | . | . | B | . | . | T | . | 1.52 | * | . | F | 1.83 | 0.71 |
| Lys | 531 | . | . | B | . | . | T | . | 1.91 | * | . | F | 2.32 | 2.58 |
| Thr | 532 | . | . | B | . | . | T | . | 1.83 | * | . | F | 2.66 | 6.96 |
| Asp | 533 | . | . | . | . | T | T | . | 1.80 | * | . | F | 3.40 | 5.89 |
| Arg | 534 | . | . | . | . | T | T | . | 2.66 | * | . | F | 3.06 | 2.55 |
| Lys | 535 | . | . | B | . | . | T | . | 2.34 | * | . | F | 2.32 | 2.95 |
| His | 536 | . | . | B | . | . | . | . | 2.09 | * | . | F | 1.78 | 2.55 |
| Phe | 537 | . | . | B | . | . | . | . | 1.70 | * | . | F | 1.44 | 2.01 |
| Asp | 538 | . | . | B | . | . | . | . | 1.67 | * | . | F | 0.65 | 0.87 |
| Thr | 539 | . | . | B | . | . | . | . | 1.21 | * | . | F | −0.25 | 0.87 |
| Pro | 540 | . | . | . | . | . | . | C | 0.87 | * | * | F | −0.05 | 1.00 |
| Phe | 541 | . | . | . | . | T | . | . | 0.61 | . | * | F | 0.45 | 0.80 |
| His | 542 | . | . | . | . | T | T | . | 0.97 | . | * | . | 0.20 | 0.58 |
| Gly | 543 | . | . | . | . | T | T | . | 0.37 | . | * | . | 0.20 | 0.37 |
| Ser | 544 | . | . | . | . | T | T | . | 0.39 | . | * | . | 0.20 | 0.43 |
| Trp | 545 | . | . | . | . | T | T | . | 0.26 | . | * | . | 0.20 | 0.33 |
| Gly | 546 | . | . | . | . | . | . | C | 0.74 | . | * | . | −0.20 | 0.33 |
| Met | 547 | . | . | . | . | T | . | . | 0.49 | . | . | . | 0.00 | 0.38 |
| Trp | 548 | . | . | . | . | T | . | . | 0.49 | . | . | . | 0.00 | 0.38 |
| Gly | 549 | . | . | . | . | . | T | C | 0.79 | . | . | . | 0.00 | 0.38 |
| Pro | 550 | . | . | . | . | T | T | . | 0.41 | . | . | F | 0.35 | 0.64 |
| Trp | 551 | . | . | . | . | T | T | . | 0.46 | * | . | F | 0.66 | 0.33 |
| Gly | 552 | . | . | . | . | T | T | . | 1.17 | * | . | F | 1.27 | 0.44 |
| Asp | 553 | . | . | . | . | T | . | . | 1.14 | * | . | F | 1.98 | 0.56 |
| Cys | 554 | . | . | . | . | T | T | . | 0.82 | * | . | F | 2.49 | 0.77 |
| Ser | 555 | . | . | . | . | T | T | . | 0.69 | * | . | F | 3.10 | 0.42 |
| Arg | 556 | . | . | . | . | T | T | . | 0.63 | * | . | F | 2.79 | 0.25 |
| Thr | 557 | . | . | . | . | T | T | . | 0.63 | * | . | F | 2.18 | 0.46 |
| Cys | 558 | . | . | . | . | T | T | . | −0.22 | * | . | F | 1.87 | 0.34 |
| Gly | 559 | . | . | . | . | T | T | . | 0.44 | * | . | F | 1.56 | 0.13 |
| Gly | 560 | . | . | . | . | T | T | . | 0.50 | * | . | F | 0.65 | 0.15 |
| Gly | 561 | . | . | . | . | T | T | . | 0.08 | * | * | F | 0.35 | 0.45 |
| Val | 562 | . | . | B | B | . | . | . | −0.21 | * | * | . | −0.60 | 0.65 |
| Gln | 563 | . | . | B | B | . | . | . | 0.57 | * | * | . | −0.60 | 0.65 |
| Tyr | 564 | . | . | B | B | . | . | . | 0.91 | * | * | . | −0.15 | 1.29 |
| Thr | 565 | . | . | B | B | . | . | . | 0.59 | * | * | . | 0.79 | 3.01 |
| Met | 566 | . | . | B | B | . | . | . | 0.93 | * | * | . | 0.98 | 0.93 |
| Arg | 567 | . | . | B | B | . | . | . | 1.79 | * | * | . | 1.62 | 0.99 |
| Glu | 568 | . | . | . | . | T | . | . | 1.58 | * | * | F | 2.86 | 1.11 |
| Cys | 569 | . | . | . | . | T | T | . | 0.97 | * | . | F | 3.40 | 1.73 |
| Asp | 570 | . | . | . | . | T | T | . | 1.07 | * | .. | F | 2.91 | 0.66 |
| Asn | 571 | . | . | . | . | . | T | C | 1.71 | * | . | F | 2.37 | 0.59 |
| Pro | 572 | . | . | . | . | . | T | C | 1.60 | * | . | F | 2.52 | 2.18 |
| Val | 573 | . | . | . | . | . | . | C | 1.26 | * | . | F | 2.32 | 2.10 |
| Pro | 574 | . | . | . | . | T | T | . | 1.58 | * | . | F | 2.42 | 1.29 |
| Lys | 575 | . | . | . | . | T | T | . | 1.62 | * | . | F | 2.61 | 0.83 |
| Asn | 576 | . | . | . | . | T | T | . | 1.38 | * | . | F | 3.40 | 2.23 |
| Gly | 577 | . | . | . | . | T | T | . | 0.92 | * | . | F | 3.06 | 2.26 |
| Gly | 578 | . | . | . | . | T | T | . | 1.78 | * | . | F | 2.27 | 0.61 |
| Lys | 579 | . | . | B | . | . | T | . | 1.64 | . | . | F | 1.53 | 0.65 |
| Tyr | 580 | . | . | B | . | . | T | . | 1.64 | . | . | F | 1.19 | 0.65 |
| Cys | 581 | . | . | B | . | . | T | . | 1.76 | . | . | F | 1.30 | 1.32 |
| Glu | 582 | . | . | B | . | . | . | . | 1.24 | . | * | F | 1.10 | 1.29 |
| Gly | 583 | . | . | B | B | . | . | . | 1.70 | . | * | F | 0.75 | 0.61 |
| Lys | 584 | . | . | B | B | . | . | . | 1.41 | . | * | F | 0.90 | 2.24 |
| Arg | 585 | . | . | B | B | . | . | . | 1.77 | . | * | F | 1.15 | 2.02 |
| Tyr | 586 | . | . | B | B | . | . | . | 2.13 | . | * | . | 1.25 | 4.01 |
| Arg | 587 | . | . | B | B | . | . | . | 1.47 | * | * | . | 1.50 | 2.68 |
| Tyr | 588 | . | . | B | . | . | T | . | 1.81 | * | * | . | 2.00 | 0.73 |
| Arg | 589 | . | . | . | . | T | T | . | 0.96 | * | * | . | 2.50 | 1.59 |
| Ser | 590 | . | . | . | . | T | T | . | 0.84 | * | * | . | 2.10 | 0.67 |
| Cys | 591 | . | . | . | . | T | T | . | 1.70 | . | * | . | 1.85 | 0.74 |
| Asn | 592 | . | A | . | . | T | . | . | 0.92 | . | * | . | 1.50 | 0.63 |
| Leu | 593 | . | A | B | . | . | . | . | 0.96 | . | . | . | 0.89 | 0.25 |
| Glu | 594 | . | A | B | . | . | . | . | 0.84 | . | . | F | 1.13 | 0.73 |
| Asp | 595 | . | A | . | . | T | . | . | 1.17 | . | . | F | 2.17 | 0.76 |
| Cys | 596 | . | . | B | . | . | T | . | 1.81 | . | . | F | 2.66 | 1.48 |
| Pro | 597 | . | . | . | . | T | T | . | 1.47 | * | * | F | 3.40 | 1.37 |
| Asp | 598 | . | . | . | . | T | T | . | 2.32 | * | * | F | 2.91 | 0.81 |
| Asn | 599 | . | . | . | . | T | T | . | 2.01 | * | . | F | 3.02 | 3.03 |
| Asn | 600 | . | . | . | . | T | T | . | 1.31 | * | . | F | 2.98 | 2.83 |
| Gly | 601 | . | . | . | . | T | T | . | 2.09 | * | . | F | 2.94 | 1.47 |
| Lys | 602 | . | . | . | . | . | T | C | 2.30 | * | * | F | 2.70 | 1.79 |
| Thr | 603 | . | . | . | . | . | T | C | 2.30 | * | . | F | 3.00 | 1.92 |
| Phe | 604 | A | A | . | . | . | . | . | 2.30 | * | . | F | 2.10 | 3.37 |
| Arg | 605 | A | A | . | . | . | . | . | 1.63 | . | . | F | 1.80 | 2.91 |
| Glu | 606 | A | A | . | . | . | . | . | 1.98 | * | . | F | 1.50 | 1.08 |
| Glu | 607 | A | A | . | . | . | . | . | 1.34 | * | . | F | 1.20 | 2.17 |
| Gln | 608 | A | A | . | . | . | . | . | 1.62 | * | . | F | 0.90 | 1.12 |
| Cys | 609 | A | A | . | . | . | . | . | 2.32 | * | * | . | 0.60 | 0.88 |
| Glu | 610 | A | A | . | . | . | . | . | 2.21 | . | * | . | 0.60 | 0.81 |
| Ala | 611 | A | A | . | . | . | . | . | 1.51 | . | * | . | 0.60 | 0.81 |
| His | 612 | A | A | . | . | . | . | . | 1.21 | * | . | . | 0.45 | 1.32 |
| Asn | 613 | A | A | . | . | . | . | . | 1.26 | * | * | . | 0.45 | 1.02 |
| Glu | 614 | A | A | . | . | . | . | . | 1.33 | * | . | . | 0.45 | 2.02 |
| Phe | 615 | A | A | . | . | . | . | . | 1.03 | * | * | F | 0.60 | 1.50 |
| Ser | 616 | A | A | . | . | . | . | . | 0.92 | . | . | F | 0.90 | 1.25 |
| Lys | 617 | A | A | . | . | . | . | . | 0.61 | . | . | F | 0.45 | 0.62 |
| Ala | 618 | . | A | . | . | T | . | . | 0.31 | . | . | F | 0.25 | 0.71 |
| Ser | 619 | . | A | . | . | T | . | . | −0.03 | . | . | F | 0.85 | 0.71 |
| Phe | 620 | . | . | . | . | T | . | . | 0.46 | . | . | F | 1.26 | 0.35 |
| Gly | 621 | . | . | . | . | T | T | . | 0.17 | . | . | F | 1.07 | 0.54 |
| Ser | 622 | . | . | . | . | . | T | C | −0.73 | . | * | F | 1.08 | 0.41 |
| Gly | 623 | . | . | . | . | . | T | C | −0.14 | . | . | F | 0.99 | 0.35 |
| Pro | 624 | . | . | . | . | . | T | C | −0.13 | . | . | F | 2.10 | 0.61 |
| Ala | 625 | . | A | . | . | . | . | C | −0.32 | . | . | F | 0.89 | 0.48 |
| Val | 626 | . | A | B | . | . | . | . | −0.19 | * | . | . | 0.03 | 0.34 |
| Glu | 627 | . | A | B | . | . | . | . | 0.16 | * | . | . | −0.18 | 0.34 |
| Trp | 628 | . | A | B | . | . | . | . | 0.26 | * | . | . | −0.09 | 0.67 |
| Ile | 629 | . | . | B | . | . | . | . | −0.12 | * | . | . | −0.25 | 1.42 |
| Pro | 630 | . | . | B | . | . | T | . | 0.12 | * | . | . | 0.10 | 0.83 |
| Lys | 631 | . | . | . | . | T | T | . | 0.12 | * | . | . | 0.20 | 0.78 |
| Tyr | 632 | . | . | . | . | T | T | . | −0.18 | * | . | . | 0.20 | 0.82 |
| Ala | 633 | . | . | . | . | T | T | . | −0.10 | * | . | . | 0.84 | 0.71 |
| Gly | 634 | . | . | . | . | T | . | . | 0.83 | * | . | . | 0.98 | 0.55 |
| Val | 635 | . | . | B | . | . | . | . | 1.04 | . | * | . | 0.92 | 0.70 |
| Ser | 636 | . | . | B | . | . | T | . | 1.11 | . | * | F | 2.66 | 1.17 |
| Pro | 637 | . | . | . | . | T | T | . | 0.69 | . | * | F | 3.40 | 2.31 |
| Lys | 638 | . | . | . | . | T | T | . | 1.32 | . | * | F | 3.06 | 1.67 |
| Asp | 639 | . | . | . | . | T | T | . | 0.86 | . | * | F | 2.72 | 2.49 |
| Arg | 640 | A | A | . | . | . | . | . | 0.82 | . | * | F | 1.58 | 1.33 |
| Cys | 641 | A | A | . | . | . | . | . | 0.46 | * | * | F | 1.09 | 0.46 |
| Lys | 642 | . | A | B | . | . | . | . | 0.67 | * | * | . | 0.30 | 0.15 |
| Leu | 643 | . | A | B | . | . | . | . | 0.03 | . | * | . | 0.30 | 0.13 |
| Ile | 644 | . | A | B | . | . | . | . | 0.08 | . | * | . | −0.60 | 0.25 |
| Cys | 645 | . | A | B | . | . | . | . | −0.38 | . | * | . | 0.30 | 0.25 |
| Gln | 646 | . | A | B | . | . | . | . | −0.60 | * | * | . | −0.30 | 0.30 |
| Ala | 647 | . | A | B | . | . | . | . | −0.99 | * | * | . | −0.30 | 0.30 |
| Lys | 648 | . | A | B | . | . | . | . | −0.42 | * | * | F | −0.15 | 0.55 |
| Gly | 649 | . | . | . | . | T | T | . | −0.23 | * | . | F | 0.65 | 0.50 |
| Ile | 650 | . | . | . | . | T | T | . | −0.27 | . | * | . | 0.20 | 0.43 |
| Gly | 651 | . | . | B | . | . | T | . | −1.12 | . | * | . | −0.20 | 0.18 |
| Tyr | 652 | . | . | B | . | . | T | . | −1.34 | . | . | . | −0.20 | 0.14 |
| Phe | 653 | . | . | B | B | . | . | . | −1.39 | . | . | . | −0.60 | 0.16 |
| Phe | 654 | . | . | B | B | . | . | . | −1.26 | . | * | . | −0.60 | 0.29 |
| Val | 655 | . | . | B | B | . | . | . | −0.32 | . | * | . | −0.60 | 0.28 |
| Leu | 656 | . | . | B | B | . | . | . | −0.83 | . | * | . | −0.60 | 0.65 |
| Gln | 657 | . | . | B | . | . | T | . | −1.44 | . | . | . | −0.20 | 0.56 |
| Pro | 658 | . | . | B | . | . | T | . | −0.74 | * | . | F | −0.05 | 0.56 |
| Lys | 659 | . | . | . | . | T | T | . | −0.39 | . | * | F | 1.40 | 1.13 |
| Val | 660 | . | . | B | . | . | T | . | 0.16 | . | . | F | 0.85 | 0.65 |
| Val | 661 | . | . | B | . | . | T | . | 0.76 | . | * | F | 0.85 | 0.60 |
| Asp | 662 | . | . | B | . | . | T | . | 0.09 | . | . | F | 1.06 | 0.47 |
| Gly | 663 | . | . | B | . | . | T | . | 0.00 | * | . | F | 0.67 | 0.34 |
| Thr | 664 | . | . | B | . | . | T | . | −0.26 | * | . | F | 1.48 | 0.61 |
| Pro | 665 | . | . | B | . | . | . | . | 0.60 | . | . | F | 1.49 | 0.56 |
| Cys | 666 | . | . | . | . | T | . | . | 1.16 | . | . | F | 2.10 | 0.95 |
| Ser | 667 | . | . | . | . | . | T | C | 0.84 | . | . | F | 1.89 | 0.88 |
| Pro | 668 | . | . | . | . | T | T | . | 0.89 | . | . | F | 1.88 | 0.82 |
| Asp | 669 | . | . | . | . | T | T | . | 0.34 | . | . | F | 1.82 | 2.06 |
| Ser | 670 | . | . | . | . | T | T | . | −0.11 | . | . | F | 1.61 | 1.14 |
| Thr | 671 | . | . | . | B | T | . | . | −0.30 | . | * | F | 0.85 | 0.39 |
| Ser | 672 | . | . | B | B | . | . | . | 0.00 | . | * | F | −0.15 | 0.18 |
| Val | 673 | . | . | B | B | . | . | . | −0.13 | . | * | . | −0.60 | 0.23 |
| Cys | 674 | . | . | B | B | . | . | . | −0.13 | . | * | . | −0.60 | 0.16 |
| Val | 675 | . | . | B | B | . | . | . | −0.50 | . | * | . | −0.60 | 0.20 |
| Gln | 676 | . | . | B | B | . | . | . | −1.04 | . | * | F | −0.45 | 0.15 |
| Gly | 677 | . | . | B | B | . | . | . | −0.70 | . | * | F | −0.45 | 0.20 |
| Gln | 678 | . | . | B | B | . | . | . | −0.43 | . | * | F | −0.15 | 0.54 |
| Cys | 679 | . | . | B | B | . | . | . | −0.11 | . | . | . | 0.30 | 0.32 |
| Val | 680 | . | . | B | B | . | . | . | 0.08 | * | * | . | 0.30 | 0.32 |
| Lys | 681 | . | . | B | . | . | T | . | 0.08 | * | . | . | 0.10 | 0.10 |
| Ala | 682 | . | . | B | . | . | T | . | 0.53 | * | . | . | 0.70 | 0.30 |
| Gly | 683 | . | . | B | . | . | T | . | −0.36 | * | . | . | 1.00 | 0.80 |
| Cys | 684 | . | . | B | . | . | T | . | −0.58 | * | . | . | 1.00 | 0.28 |
| Asp | 685 | A | . | . | B | . | . | . | 0.28 | * | . | . | 0.30 | 0.20 |
| Arg | 686 | A | . | . | B | . | . | . | −0.07 | * | . | . | 0.60 | 0.33 |
| Ile | 687 | A | . | . | B | . | . | . | 0.57 | * | . | . | 0.60 | 0.82 |
| Ile | 688 | A | . | . | B | . | . | . | 0.96 | * | . | F | 0.75 | 0.99 |
| Asp | 689 | A | . | . | . | . | T | . | 1.67 | * | * | F | 1.30 | 1.01 |
| Ser | 690 | A | . | . | . | . | T | . | 0.97 | * | * | F | 1.30 | 2.88 |
| Lys | 691 | A | . | . | . | . | T | . | 0.86 | * | . | F | 1.61 | 3.55 |
| Lys | 692 | . | . | . | . | T | T | . | 1.79 | * | * | F | 2.32 | 3.55 |
| Lys | 693 | . | . | . | . | T | . | . | 2.01 | * | * | F | 2.43 | 5.30 |
| Phe | 694 | . | . | . | . | T | . | . | 1.67 | * | * | F | 2.74 | 1.42 |
| Asp | 695 | . | . | . | . | T | T | . | 1.11 | * | . | F | 3.10 | 0.70 |
| Lys | 696 | . | . | B | . | . | T | . | 0.40 | * | . | F | 2.39 | 0.26 |
| Cys | 697 | . | . | B | . | . | T | . | 0.01 | * | . | . | 1.63 | 0.16 |
| Gly | 698 | . | . | B | . | . | T | . | −0.38 | * | . | . | 1.32 | 0.10 |
| Val | 699 | . | . | B | . | . | . | . | 0.32 | * | . | . | 0.21 | 0.05 |
| Cys | 700 | . | . | . | . | T | . | . | −0.02 | . | . | . | 0.00 | 0.14 |
| Gly | 701 | . | . | . | . | T | T | . | −0.37 | . | . | F | 0.65 | 0.14 |
| Gly | 702 | . | . | . | . | T | T | . | −0.01 | . | . | F | 0.65 | 0.26 |
| Asn | 703 | . | . | . | . | T | T | . | −0.33 | . | . | F | 0.65 | 0.69 |
| Gly | 704 | . | . | . | . | T | T | . | 0.57 | . | . | F | 0.65 | 0.37 |
| Ser | 705 | . | . | . | . | T | T | . | 1.28 | . | . | F | 1.25 | 0.76 |
| Thr | 706 | . | . | B | . | . | T | . | 0.73 | . | . | F | 1.41 | 0.94 |
| Cys | 707 | . | . | B | . | . | T | . | 0.78 | . | * | F | 1.37 | 0.67 |
| Lys | 708 | . | . | B | . | . | T | . | 0.43 | . | * | F | 1.63 | 0.67 |
| Lys | 709 | . | . | B | . | . | . | . | 0.48 | * | * | F | 1.69 | 0.46 |
| Ile | 710 | . | . | B | . | . | T | . | −0.08 | * | * | F | 2.60 | 1.14 |
| Ser | 711 | . | . | B | . | . | T | . | −0.08 | * | * | F | 1.89 | 0.42 |
| Gly | 712 | . | . | B | . | . | T | . | 0.29 | * | * | F | 1.03 | 0.31 |
| Ser | 713 | . | . | B | . | . | T | . | −0.34 | * | * | F | 0.77 | 0.58 |
| Val | 714 | . | . | B | B | . | . | . | −0.34 | . | * | F | 0.11 | 0.44 |
| Thr | 715 | . | . | B | B | . | . | . | 0.33 | . | . | F | 0.73 | 0.89 |
| Ser | 716 | . | . | B | B | . | . | . | 0.29 | . | . | F | 1.16 | 1.03 |
| Ala | 717 | . | . | B | . | . | . | . | 0.39 | . | . | F | 1.64 | 1.37 |
| Lys | 718 | . | . | . | . | . | T | C | 0.66 | . | . | F | 2.32 | 1.49 |
| Pro | 719 | . | . | . | . | T | T | . | 1.51 | * | . | F | 2.80 | 1.51 |
| Gly | 720 | . | . | . | . | T | T | . | 0.93 | * | . | F | 2.52 | 2.50 |
| Tyr | 721 | . | . | B | . | . | T | . | 0.34 | * | . | . | 1.54 | 0.88 |
| His | 722 | . | . | B | B | . | . | . | 0.62 | * | . | . | −0.04 | 0.40 |
| Asp | 723 | . | . | B | B | . | . | . | −0.31 | * | . | . | −0.32 | 0.58 |
| Ile | 724 | . | . | B | B | . | . | . | −0.31 | * | . | . | −0.60 | 0.26 |
| Ile | 725 | . | . | B | B | . | . | . | −0.28 | * | . | . | −0.60 | 0.29 |
| Thr | 726 | . | . | B | B | . | . | . | −0.38 | * | . | . | −0.60 | 0.25 |
| Ile | 727 | . | . | B | . | . | T | . | −0.93 | * | . | . | −0.20 | 0.36 |
| Pro | 728 | . | . | B | . | . | T | . | −1.24 | * | . | F | −0.05 | 0.52 |
| Thr | 729 | . | . | . | . | . | T | C | −0.36 | * | . | F | 0.15 | 0.52 |
| Gly | 730 | . | . | . | . | . | T | C | −0.36 | . | * | F | 0.30 | 1.19 |
| Ala | 731 | . | . | . | B | . | . | C | −0.04 | . | * | F | −0.25 | 0.54 |
| Thr | 732 | . | . | . | B | . | . | C | −0.01 | . | * | F | 0.65 | 0.65 |
| Asn | 733 | . | . | B | B | . | . | . | 0.24 | . | * | F | −0.15 | 0.48 |
| Ile | 734 | . | . | B | B | . | . | . | 0.56 | . | * | F | 0.45 | 0.96 |
| Glu | 735 | . | . | B | B | . | . | . | 1.01 | . | * | F | 0.60 | 1.15 |
| Val | 736 | . | . | B | B | . | . | . | 1.60 | . | * | F | 0.90 | 1.40 |
| Lys | 737 | . | . | B | B | . | . | . | 1.91 | . | * | F | 1.24 | 3.21 |
| Gln | 738 | . | . | B | . | . | . | . | 2.02 | . | * | F | 1.78 | 3.21 |
| Arg | 739 | . | . | B | . | . | . | . | 2.57 | * | * | F | 2.12 | 8.48 |
| Asn | 740 | . | . | B | . | . | T | . | 2.27 | * | * | F | 2.66 | 4.20 |
| Gln | 741 | . | . | . | . | T | T | . | 3.23 | * | * | F | 3.40 | 3.25 |
| Arg | 742 | . | . | . | . | T | T | . | 3.19 | * | . | F | 3.06 | 3.25 |
| Gly | 743 | . | . | . | . | T | T | . | 3.19 | * | . | F | 3.00 | 3.25 |
| Ser | 744 | . | . | . | . | T | . | . | 2.73 | * | . | F | 2.74 | 3.02 |
| Arg | 745 | . | . | . | . | . | . | C | 2.43 | * | * | F | 2.48 | 1.52 |
| Asn | 746 | . | . | . | . | T | T | . | 1.73 | * | . | F | 2.82 | 2.06 |
| Asn | 747 | . | . | . | . | T | T | . | 0.81 | * | . | F | 2.80 | 1.33 |
| Gly | 748 | . | . | . | . | . | T | C | 0.57 | . | * | F | 1.57 | 0.56 |
| Ser | 749 | . | . | B | . | . | T | . | −0.02 | . | * | F | 0.79 | 0.35 |
| Phe | 750 | . | A | B | . | . | . | . | −0.09 | . | * | . | −0.04 | 0.15 |
| Leu | 751 | . | A | B | . | . | . | . | −0.68 | . | . | . | −0.32 | 0.31 |
| Ala | 752 | . | A | B | . | . | . | . | −1.27 | * | . | . | −0.60 | 0.23 |
| Ile | 753 | . | A | B | . | . | . | . | −0.92 | . | . | . | −0.60 | 0.27 |
| Lys | 754 | A | A | . | . | . | . | . | −0.97 | . | . | . | 0.30 | 0.55 |
| Ala | 755 | A | A | . | . | . | . | . | −0.58 | . | . | . | 0.30 | 0.54 |
| Ala | 756 | A | A | . | . | . | . | . | −0.01 | . | . | F | 0.60 | 1.12 |
| Asp | 757 | A | . | . | . | . | T | . | −0.31 | . | . | F | 0.85 | 0.87 |
| Gly | 758 | . | . | B | . | . | T | . | −0.23 | . | * | F | 0.25 | 0.61 |
| Thr | 759 | . | . | B | . | . | T | . | −0.28 | . | . | F | −0.05 | 0.50 |
| Tyr | 760 | . | . | B | . | . | T | . | −0.03 | . | * | . | −0.20 | 0.48 |
| Ile | 761 | . | . | B | . | . | . | . | 0.56 | . | * | . | −0.40 | 0.48 |
| Leu | 762 | . | . | B | . | . | . | . | 0.31 | . | * | . | −0.40 | 0.55 |
| Asn | 763 | . | . | B | . | . | T | . | 0.34 | . | * | F | −0.50 | 0.55 |
| Gly | 764 | . | . | . | . | T | T | . | −0.16 | . | * | F | 0.50 | 1.14 |
| Asp | 765 | . | . | . | . | T | T | . | −0.21 | . | * | F | 0.50 | 1.14 |
| Tyr | 766 | . | . | . | . | . | T | C | 0.37 | . | * | F | 0.45 | 0.95 |
| Thr | 767 | . | . | B | B | . | . | . | 0.37 | . | * | . | −0.15 | 1.38 |
| Leu | 768 | . | . | B | B | . | . | . | 0.37 | * | * | . | −0.60 | 0.68 |
| Ser | 769 | . | . | B | B | . | . | . | 0.71 | * | . | F | −0.45 | 0.75 |
| Thr | 770 | . | . | B | B | . | . | . | 0.71 | * | * | F | −0.15 | 0.90 |
| Leu | 771 | A | . | . | B | . | . | . | 0.07 | * | . | F | 0.60 | 1.83 |
| Glu | 772 | A | . | . | B | . | . | . | −0.22 | * | . | F | 0.45 | 0.96 |
| Gln | 773 | A | . | . | B | . | . | . | 0.34 | * | * | F | 0.45 | 0.66 |
| Asp | 774 | A | . | . | B | . | . | . | 0.69 | . | * | F | 0.00 | 1.25 |
| Ile | 775 | A | . | . | B | . | . | . | 0.66 | . | * | . | 0.75 | 1.44 |
| Met | 776 | A | . | . | B | . | . | . | 0.61 | . | * | . | 0.30 | 0.82 |
| Tyr | 777 | . | . | B | B | . | . | . | −0.24 | . | * | . | −0.30 | 0.37 |
| Lys | 778 | . | . | B | B | . | . | . | −1.06 | . | * | . | −0.60 | 0.39 |
| Gly | 779 | . | . | B | B | . | . | . | −0.94 | . | * | . | −0.60 | 0.32 |
| Val | 780 | . | . | B | B | . | . | . | −0.30 | . | * | . | −0.30 | 0.40 |
| Val | 781 | . | . | B | B | . | . | . | 0.00 | . | * | . | −0.30 | 0.32 |
| Leu | 782 | . | . | B | B | . | . | . | −0.10 | . | * | . | −0.60 | 0.43 |
| Arg | 783 | . | . | B | B | . | . | . | −0.44 | . | * | . | −0.60 | 0.57 |
| Tyr | 784 | . | . | B | . | . | T | . | −0.40 | * | * | . | 0.25 | 1.03 |
| Ser | 785 | . | . | . | . | T | T | . | −0.13 | . | * | F | 0.80 | 1.68 |
| Gly | 786 | . | . | . | . | . | T | C | 0.13 | * | * | F | 1.05 | 0.86 |
| Ser | 787 | . | . | . | . | . | T | C | 0.13 | . | * | F | 0.45 | 0.56 |
| Ser | 788 | . | A | . | . | . | . | C | 0.02 | . | * | F | 0.05 | 0.34 |
| Ala | 789 | A | A | . | . | . | . | . | 0.38 | * | . | F | 0.45 | 0.60 |
| Ala | 790 | A | A | . | . | . | . | . | −0.21 | * | * | . | 0.60 | 0.88 |
| Leu | 791 | A | A | . | . | . | . | . | 0.24 | * | * | . | 0.30 | 0.46 |
| Glu | 792 | A | A | . | . | . | . | . | 0.24 | * | * | . | 0.60 | 0.89 |
| Arg | 793 | . | A | B | B | . | . | . | −0.16 | * | * | F | 0.90 | 1.18 |
| Ile | 794 | A | A | . | B | . | . | . | 0.13 | * | * | F | 0.60 | 1.24 |
| Arg | 795 | A | A | . | B | . | . | . | 0.51 | * | * | F | 0.75 | 0.96 |
| Ser | 796 | . | A | . | . | T | . | . | 0.51 | . | * | F | 1.13 | 0.76 |
| Phe | 797 | . | . | . | . | . | . | C | 0.56 | . | * | F | 0.81 | 0.89 |
| Ser | 798 | . | . | . | . | . | T | C | 0.44 | . | * | F | 1.89 | 0.91 |
| Pro | 799 | . | . | . | . | . | T | C | 1.12 | * | * | F | 2.32 | 1.17 |
| Leu | 800 | . | . | . | . | T | T | . | 0.20 | * | * | F | 2.80 | 2.10 |
| Lys | 801 | . | . | . | . | . | T | C | 0.19 | * | * | F | 2.32 | 1.29 |
| Glu | 802 | . | . | . | . | . | . | C | 0.00 | . | * | F | 1.84 | 1.20 |
| Pro | 803 | A | . | . | B | . | . | . | 0.30 | . | * | F | 1.16 | 1.02 |
| Leu | 804 | A | . | . | B | . | . | . | −0.34 | . | * | F | 0.73 | 0.89 |
| Thr | 805 | . | . | B | B | . | . | . | −0.34 | . | * | . | −0.30 | 0.38 |
| Ile | 806 | . | . | B | B | . | . | . | −0.70 | . | * | . | −0.60 | 0.20 |
| Gln | 807 | . | . | B | B | . | . | . | −1.56 | . | . | . | −0.60 | 0.35 |
| Val | 808 | . | . | B | B | . | . | . | −1.69 | . | * | . | −0.60 | 0.18 |
| Leu | 809 | . | . | B | B | . | . | . | −0.88 | . | * | . | −0.60 | 0.26 |
| Thr | 810 | . | . | B | B | . | . | . | −1.16 | . | . | . | −0.60 | 0.24 |
| Val | 811 | . | . | B | B | . | . | . | −1.08 | . | * | . | −0.60 | 0.33 |
| Gly | 812 | . | . | B | B | . | . | . | −0.97 | * | * | . | −0.60 | 0.33 |
| Asn | 813 | A | . | . | . | . | . | . | −0.32 | * | * | . | 0.12 | 0.44 |
| Ala | 814 | A | . | . | . | . | . | . | 0.53 | * | * | . | 0.34 | 0.92 |
| Leu | 815 | A | . | . | . | . | . | . | −0.04 | * | * | F | 1.76 | 1.86 |
| Arg | 816 | . | . | B | . | . | . | . | 0.86 | * | * | F | 1.53 | 0.81 |
| Pro | 817 | . | . | B | . | . | . | . | 0.96 | * | * | F | 2.20 | 1.61 |
| Lys | 818 | . | . | B | B | . | . | . | 0.64 | * | * | F | 1.48 | 3.05 |
| Ile | 819 | . | . | B | B | . | . | . | 0.99 | . | * | F | 1.56 | 2.25 |
| Lys | 820 | . | . | B | B | . | . | . | 1.10 | * | * | F | 0.44 | 2.28 |
| Tyr | 821 | . | . | B | B | . | . | . | 0.13 | * | * | . | −0.38 | 0.99 |
| Thr | 822 | . | . | B | B | . | . | . | 0.39 | . | * | . | −0.45 | 1.04 |
| Tyr | 823 | A | . | . | B | . | . | . | 0.39 | . | * | . | −0.45 | 1.04 |
| Phe | 824 | A | . | . | B | . | . | . | 1.32 | . | * | . | −0.45 | 1.33 |
| Val | 825 | A | . | . | B | . | . | . | 1.32 | . | . | . | 0.45 | 1.85 |
| Lys | 826 | A | . | . | B | . | . | . | 1.57 | . | . | F | 0.90 | 2.36 |
| Lys | 827 | A | A | . | . | . | . | . | 1.58 | * | . | F | 0.90 | 4.71 |
| Lys | 828 | A | A | . | . | . | . | . | 1.12 | * | . | F | 0.90 | 8.51 |
| Lys | 829 | A | A | . | . | . | . | . | 1.82 | * | . | F | 0.90 | 3.68 |
| Glu | 830 | A | A | . | . | . | . | . | 2.09 | * | . | F | 0.90 | 2.96 |
| Ser | 831 | A | A | . | . | . | . | . | 1.16 | * | . | F | 0.90 | 1.50 |
| Phe | 832 | A | A | . | . | . | . | . | 0.90 | . | . | . | 0.30 | 0.52 |
| Asn | 833 | . | A | B | . | . | . | . | 0.54 | * | . | . | −0.30 | 0.47 |
| Ala | 834 | . | . | B | . | . | . | . | −0.20 | * | * | . | −0.40 | 0.50 |
| Ile | 835 | . | . | . | . | . | . | C | −0.50 | * | . | . | −0.20 | 0.50 |
| Pro | 836 | . | . | . | . | . | T | C | −0.79 | . | . | . | 0.00 | 0.42 |
| Thr | 837 | . | . | . | . | T | T | . | −0.38 | * | * | . | 0.20 | 0.42 |
| Phe | 838 | A | . | . | . | . | T | . | −1.23 | * | . | . | −0.20 | 0.63 |
| Ser | 839 | . | . | . | . | . | T | C | −1.53 | * | . | . | 0.00 | 0.30 |
| Ala | 840 | . | A | B | B | . | . | . | −0.64 | * | . | . | −0.60 | 0.15 |
| Trp | 841 | . | A | B | B | . | . | . | −0.43 | . | . | . | −0.60 | 0.29 |
| Val | 842 | A | A | . | B | . | . | . | −0.41 | . | . | . | −0.30 | 0.38 |
| Ile | 843 | A | A | . | B | . | . | . | −0.06 | * | . | . | −0.60 | 0.40 |
| Glu | 844 | A | A | . | B | . | . | . | 0.24 | * | . | . | −0.60 | 0.37 |
| Glu | 845 | A | A | . | . | . | . | . | 0.17 | * | . | . | 0.30 | 0.87 |
| Trp | 846 | A | A | . | . | . | . | . | 0.16 | * | . | . | 0.61 | 0.66 |
| Gly | 847 | A | A | . | . | . | . | . | 1.06 | * | . | F | 1.37 | 0.51 |
| Glu | 848 | . | A | . | . | T | . | . | 1.64 | * | . | F | 2.08 | 0.59 |
| Cys | 849 | . | A | . | . | T | . | . | 0.98 | * | . | F | 2.09 | 0.76 |
| Ser | 850 | . | . | . | . | T | T | . | 0.98 | . | . | F | 3.10 | 0.41 |
| Lys | 851 | . | . | . | . | T | T | . | 0.46 | . | . | F | 2.79 | 0.41 |
| Ser | 852 | . | . | . | . | T | T | . | 0.46 | . | . | F | 2.18 | 0.63 |
| Cys | 853 | . | . | . | . | T | T | . | 0.17 | * | * | . | 2.02 | 0.47 |
| Glu | 854 | A | A | . | . | . | . | . | 0.83 | * | . | . | 0.61 | 0.24 |
| Leu | 855 | A | A | . | . | . | . | . | 1.24 | . | . | . | −0.30 | 0.32 |
| Gly | 856 | . | A | . | . | . | . | . | 1.31 | . | * | . | 0.85 | 1.16 |
| Trp | 857 | A | A | . | . | . | . | . | 0.80 | * | * | . | 0.75 | 1.31 |
| Gln | 858 | A | A | . | . | . | . | . | 0.61 | * | * | . | −0.15 | 1.31 |
| Arg | 859 | A | A | . | . | . | . | . | 0.61 | * | * | . | −0.30 | 0.98 |
| Arg | 860 | . | A | B | . | . | . | . | 0.76 | . | * | . | 0.45 | 1.61 |
| Leu | 861 | . | A | B | . | . | . | . | 1.21 | * | . | . | 0.60 | 0.50 |
| Val | 862 | . | A | B | . | . | . | . | 1.50 | * | . | . | 0.60 | 0.50 |
| Glu | 863 | . | A | B | . | . | . | . | 0.61 | . | . | . | 0.94 | 0.43 |
| Cys | 864 | . | A | B | . | . | . | . | 0.50 | . | . | . | 0.98 | 0.36 |
| Arg | 865 | . | A | . | . | T | . | . | 0.04 | . | . | F | 2.17 | 0.78 |
| Asp | 866 | . | . | . | . | T | T | . | 0.86 | . | . | F | 2.91 | 0.45 |
| Ile | 867 | . | . | . | . | T | T | . | 1.50 | . | . | F | 3.40 | 1.45 |
| Asn | 868 | . | . | . | . | T | T | . | 0.91 | . | . | F | 3.06 | 1.14 |
| Gly | 869 | . | . | . | . | . | T | C | 1.28 | . | . | F | 2.07 | 0.69 |
| Gln | 870 | . | . | . | . | . | T | C | 1.17 | . | * | F | 1.28 | 1.32 |
| Pro | 871 | . | . | . | . | . | T | C | 0.50 | . | * | F | 1.54 | 1.42 |
| Ala | 872 | . | . | . | . | . | T | C | 0.80 | . | * | F | 1.05 | 0.77 |
| Ser | 873 | A | . | . | . | . | T | . | 0.84 | * | . | F | 0.85 | 0.45 |
| Glu | 874 | A | A | . | . | . | . | . | 1.19 | * | . | F | 0.75 | 0.58 |
| Cys | 875 | A | A | . | . | . | . | . | 0.33 | * | . | . | 0.60 | 1.00 |
| Ala | 876 | A | A | . | . | . | . | . | 0.59 | * | . | . | 0.60 | 0.55 |
| Lys | 877 | A | A | . | . | . | . | . | 0.97 | * | . | F | 0.75 | 0.64 |
| Glu | 878 | A | A | . | . | . | . | . | 0.68 | * | . | F | 0.90 | 1.84 |
| Val | 879 | A | A | . | . | . | . | . | 0.38 | * | . | F | 0.90 | 1.84 |
| Lys | 880 | A | A | . | . | . | . | . | 0.73 | * | . | F | 0.90 | 1.23 |
| Pro | 881 | A | . | . | . | . | T | . | 1.43 | * | . | F | 1.30 | 1.03 |
| Ala | 882 | . | . | . | . | T | T | . | 1.18 | * | . | F | 2.01 | 2.71 |
| Ser | 883 | . | . | . | . | T | T | . | 0.51 | . | * | F | 2.32 | 2.10 |
| Thr | 884 | . | . | . | . | T | T | . | 0.78 | . | * | F | 2.18 | 0.73 |
| Arg | 885 | . | . | B | . | . | T | . | 0.73 | . | * | F | 2.09 | 0.73 |
| Pro | 886 | . | . | . | . | T | T | . | 0.91 | . | * | F | 3.10 | 0.91 |
| Cys | 887 | . | . | . | . | T | T | . | 1.29 | . | * | . | 2.64 | 0.85 |
| Ala | 888 | . | . | . | . | T | T | . | 0.92 | . | * | . | 2.43 | 0.67 |
| Asp | 889 | . | . | . | . | T | . | . | 1.02 | . | * | . | 1.72 | 0.23 |
| His | 890 | . | . | . | . | . | T | C | 0.91 | . | * | . | 1.51 | 0.67 |
| Pro | 891 | . | . | . | . | T | T | . | 0.83 | . | . | . | 1.65 | 1.16 |
| Cys | 892 | . | . | . | . | T | T | . | 1.50 | . | * | . | 1.00 | 0.73 |
| Pro | 893 | . | . | . | . | T | T | . | 1.28 | . | * | . | 0.60 | 0.93 |
| Gln | 894 | . | A | . | . | T | . | . | 0.93 | . | . | . | 0.10 | 0.49 |
| Trp | 895 | . | A | B | . | . | . | . | 0.97 | . | . | . | −0.40 | 0.91 |
| Gln | 896 | . | A | B | . | . | . | . | 0.89 | . | . | . | −0.05 | 1.02 |
| Leu | 897 | . | A | B | . | . | . | . | 1.26 | . | . | . | −0.60 | 0.62 |
| Gly | 898 | . | . | . | . | T | . | . | 1.17 | . | . | . | 0.00 | 0.79 |
| Glu | 899 | . | . | . | . | T | . | . | 0.50 | . | . | F | 0.45 | 0.61 |
| Trp | 900 | . | . | . | . | T | . | . | 0.49 | . | . | F | 0.45 | 0.40 |
| Ser | 901 | . | . | . | . | T | T | . | 0.53 | . | . | F | 0.65 | 0.54 |
| Ser | 902 | . | . | . | . | T | T | . | 1.03 | . | . | F | 1.25 | 0.62 |
| Cys | 903 | . | . | . | . | T | T | . | 0.71 | . | * | F | 0.65 | 0.85 |
| Ser | 904 | . | . | . | . | T | T | . | 0.37 | * | * | F | 1.25 | 0.34 |
| Lys | 905 | . | . | . | . | T | . | . | 0.70 | * | * | F | 1.05 | 0.25 |
| Thr | 906 | . | . | . | . | T | . | . | 0.66 | * | * | F | 1.69 | 0.94 |
| Cys | 907 | . | . | . | . | T | . | . | 0.71 | * | . | F | 2.03 | 0.69 |
| Gly | 908 | . | . | . | . | T | T | . | 1.42 | * | * | F | 2.27 | 0.54 |
| Lys | 909 | . | . | . | . | T | T | . | 1.77 | * | * | F | 2.61 | 0.75 |
| Gly | 910 | . | . | . | . | T | T | . | 1.83 | * | * | F | 3.40 | 2.81 |
| Tyr | 911 | . | . | . | . | T | T | . | 1.84 | . | . | F | 3.06 | 5.57 |
| Lys | 912 | . | A | B | . | . | . | . | 1.70 | * | . | F | 1.92 | 3.73 |
| Lys | 913 | . | A | B | . | . | . | . | 2.09 | * | . | F | 1.58 | 3.11 |
| Arg | 914 | . | A | B | . | . | . | . | 1.38 | * | . | F | 1.24 | 3.97 |
| Ser | 915 | . | A | B | . | . | . | . | 0.91 | * | . | F | 0.90 | 1.06 |
| Leu | 916 | . | A | B | . | . | . | . | 0.86 | * | . | F | 0.75 | 0.44 |
| Lys | 917 | . | A | B | . | . | . | . | 0.78 | * | . | . | 0.30 | 0.30 |
| Cys | 918 | . | A | B | . | . | . | . | 0.73 | * | . | . | −0.30 | 0.30 |
| Leu | 919 | . | A | B | . | . | . | . | 0.28 | * | . | . | 0.30 | 0.62 |
| Ser | 920 | . | . | B | . | . | . | . | 0.23 | . | . | . | 0.50 | 0.31 |
| His | 921 | . | . | B | . | . | T | . | 0.19 | * | . | F | 0.85 | 0.56 |
| Asp | 922 | . | . | . | . | T | T | . | −0.67 | * | . | F | 0.65 | 0.51 |
| Gly | 923 | . | . | . | . | T | T | . | −0.30 | . | . | F | 0.65 | 0.31 |
| Gly | 924 | . | . | . | . | T | T | . | 0.48 | . | . | F | 0.65 | 0.31 |
| Val | 925 | . | . | B | . | . | . | . | 0.78 | . | . | . | −0.10 | 0.25 |
| Leu | 926 | . | . | B | . | . | . | . | 0.51 | . | . | . | −0.10 | 0.44 |
| Ser | 927 | . | . | B | . | . | . | . | −0.16 | . | . | . | −0.10 | 0.59 |
| His | 928 | . | . | B | . | . | T | . | 0.19 | . | . | . | 0.10 | 0.43 |
| Glu | 929 | . | . | B | . | . | T | . | 0.32 | . | . | F | 0.85 | 0.87 |
| Ser | 930 | A | . | . | . | . | T | . | 0.37 | * | . | F | 1.30 | 1.00 |
| Cys | 931 | A | . | . | . | . | T | . | 1.22 | * | . | F | 0.85 | 0.61 |
| Asp | 932 | A | . | . | . | . | T | . | 1.57 | * | . | F | 1.15 | 0.70 |
| Pro | 933 | A | . | . | . | . | T | . | 1.39 | * | . | F | 1.30 | 1.05 |
| Leu | 934 | A | . | . | . | . | T | . | 1.43 | * | . | F | 1.30 | 3.02 |
| Lys | 935 | A | . | . | . | . | T | . | 1.70 | * | . | F | 1.30 | 3.62 |
| Lys | 936 | A | A | . | . | . | . | . | 1.67 | * | . | F | 0.90 | 3.18 |
| Pro | 937 | A | A | . | . | . | . | . | 0.78 | * | . | F | 0.90 | 3.34 |
| Lys | 938 | A | A | . | . | . | . | . | 0.99 | * | * | F | 0.90 | 1.17 |
| His | 939 | A | A | . | . | . | . | . | 1.10 | * | * | . | 0.60 | 0.98 |
| Phe | 940 | . | A | B | . | . | . | . | 0.39 | * | * | . | −0.30 | 0.55 |
| Ile | 941 | . | A | B | . | . | . | . | 0.03 | * | * | . | −0.30 | 0.15 |
| Asp | 942 | A | A | . | . | . | . | . | −0.36 | * | * | . | −0.60 | 0.16 |
| Phe | 943 | A | A | . | . | . | . | . | −0.99 | * | * | . | −0.60 | 0.18 |
| Cys | 944 | A | A | . | . | . | . | . | −0.96 | . | . | . | −0.60 | 0.26 |
| Thr | 945 | A | A | . | . | . | . | . | −0.92 | . | * | . | 0.30 | 0.27 |
| Met | 946 | A | A | . | . | . | . | . | −0.33 | . | * | . | −0.60 | 0.16 |
| Ala | 947 | A | A | . | . | . | . | . | −0.72 | . | . | . | −0.30 | 0.41 |
| Glu | 948 | A | A | . | . | . | . | . | −0.41 | . | . | . | 0.30 | 0.36 |
| Cys | 949 | A | A | . | . | . | . | . | −0.13 | . | . | . | 0.30 | 0.47 |
| Ser | 950 | A | A | . | . | . | . | . | −0.21 | . | . | . | 0.30 | 0.60 |

TABLE 2

| | | Garni . . . | Chou- . . . | Garni . . . | Chou- . . . | Garni . . . | Chou- . . . | Garni . . . | Kyte- . . . | Eisen . . . | Eisen . . . | Karpl . . . | James . . . | Emini |
|---|---|---|---|---|---|---|---|---|---|---|---|---|---|---|
| Res | Pos. | Alpha | Alpha | Beta | Beta | Turn | Turn | Coil | Hydro . . . | Alpha | Beta | Flexi . . . | Antig . . . | Surfa . . . |
| Met | 1 | . | . | B | . | . | . | . | −0.37 | . | . | . | −0.40 | 0.50 |
| Phe | 2 | . | . | B | . | . | . | . | −0.57 | . | . | . | −0.40 | 0.61 |
| Pro | 3 | . | . | B | . | . | . | . | −0.77 | . | . | . | −0.40 | 0.48 |
| Ala | 4 | . | . | . | . | . | . | C | −0.59 | . | * | . | −0.20 | 0.49 |
| Pro | 5 | . | . | . | . | . | . | C | −0.09 | . | * | . | −0.20 | 0.87 |
| Ala | 6 | . | . | . | . | . | . | C | 0.22 | * | * | . | 0.85 | 1.11 |
| Ala | 7 | . | . | . | . | . | T | C | 0.11 | * | * | . | 0.45 | 1.15 |
| Pro | 8 | A | . | . | . | . | T | . | 0.11 | * | . | . | −0.20 | 0.61 |
| Arg | 9 | . | . | . | . | T | T | . | 0.00 | * | . | . | 0.20 | 0.94 |
| Trp | 10 | . | . | B | . | . | T | . | −0.60 | * | . | . | −0.20 | 0.81 |
| Leu | 11 | . | A | B | . | . | . | . | −0.82 | * | . | . | −0.60 | 0.43 |
| Pro | 12 | . | A | B | . | . | . | . | −1.04 | * | . | . | −0.60 | 0.18 |
| Phe | 13 | . | A | B | . | . | . | . | −1.64 | * | . | . | −0.60 | 0.14 |
| Leu | 14 | A | A | . | . | . | . | . | −2.57 | * | . | . | −0.60 | 0.14 |
| Leu | 15 | A | A | . | . | . | . | . | −3.09 | . | . | . | −0.60 | 0.08 |
| Leu | 16 | A | A | . | . | . | . | . | −3.09 | . | . | . | −0.60 | 0.07 |
| Leu | 17 | A | A | . | . | . | . | . | −3.69 | . | . | . | −0.60 | 0.07 |
| Leu | 18 | A | A | . | . | . | . | . | −3.80 | . | . | . | −0.60 | 0.07 |
| Leu | 19 | A | A | . | . | . | . | . | −3.20 | . | . | . | −0.60 | 0.07 |
| Leu | 20 | A | A | . | . | . | . | . | −3.20 | . | . | . | −0.60 | 0.14 |
| Leu | 21 | A | A | . | . | . | . | . | −2.98 | * | . | . | −0.60 | 0.14 |
| Leu | 22 | . | A | B | . | . | . | . | −2.06 | * | . | . | −0.60 | 0.17 |
| Pro | 23 | . | A | B | . | . | . | . | −1.59 | * | . | . | −0.60 | 0.39 |
| Leu | 24 | A | A | . | . | . | . | . | −1.37 | * | . | . | −0.60 | 0.47 |
| Ala | 25 | A | A | . | . | . | . | . | −0.77 | * | . | . | −0.04 | 0.58 |
| Arg | 26 | . | A | B | . | . | . | . | −0.54 | * | . | . | 0.82 | 0.58 |
| Gly | 27 | . | A | B | . | . | . | . | 0.38 | . | . | F | 0.63 | 0.71 |
| Ala | 28 | . | . | B | . | . | . | . | 0.38 | . | . | F | 2.14 | 1.37 |
| Pro | 29 | . | . | . | . | . | . | C | 0.60 | . | . | F | 2.60 | 1.08 |
| Ala | 30 | . | . | B | . | . | . | . | 0.60 | . | . | F | 1.84 | 1.11 |
| Arg | 31 | . | . | B | . | . | . | . | 0.14 | . | . | F | 1.58 | 1.11 |
| Pro | 32 | . | . | B | . | . | . | . | 0.14 | . | * | F | 1.17 | 0.71 |
| Ala | 33 | . | . | B | . | . | T | . | 0.73 | . | * | F | 1.11 | 0.69 |
| Ala | 34 | A | . | . | . | . | T | . | 0.36 | . | * | F | 0.85 | 0.61 |
| Gly | 35 | . | . | . | . | . | T | C | 0.64 | . | * | F | 0.45 | 0.40 |
| Gly | 36 | . | . | . | . | . | T | C | 0.53 | . | * | F | 0.45 | 0.53 |
| Gln | 37 | A | . | . | . | . | . | . | −0.07 | . | . | F | 0.65 | 0.91 |
| Ala | 38 | . | . | B | . | . | . | . | −0.33 | . | . | F | 0.65 | 0.76 |
| Ser | 39 | . | . | B | B | B | . | . | −0.60 | . | . | F | −0.15 | 0.57 |
| Glu | 40 | . | . | B | B | B | . | . | −0.47 | . | . | F | −0.15 | 0.24 |
| Leu | 41 | . | . | B | B | B | . | . | −0.43 | . | * | . | −0.30 | 0.37 |
| Val | 42 | . | . | B | B | B | . | . | −0.32 | . | * | . | −0.30 | 0.40 |
| Val | 43 | . | . | B | B | B | . | . | −0.54 | . | * | . | 0.30 | 0.46 |
| Pro | 44 | . | . | B | B | B | . | . | −0.46 | . | * | F | −0.24 | 0.46 |
| Thr | 45 | . | . | B | B | B | . | . | −0.80 | . | * | F | 0.27 | 0.95 |
| Arg | 46 | . | . | B | B | B | . | . | −0.29 | . | * | F | 0.63 | 1.26 |
| Leu | 47 | . | . | . | . | . | T | C | −0.02 | . | * | F | 2.04 | 1.10 |
| Pro | 48 | . | . | . | . | . | T | C | 0.49 | * | * | F | 2.10 | 0.77 |
| Gly | 49 | . | . | . | . | . | T | C | 0.70 | * | * | F | 1.89 | 0.39 |
| Ser | 50 | . | . | . | . | . | T | C | 0.20 | * | * | F | 1.68 | 0.81 |
| Ala | 51 | A | A | . | . | . | . | . | −0.50 | * | * | F | 0.87 | 0.43 |
| Gly | 52 | A | A | . | . | . | . | . | −0.50 | . | . | F | 0.66 | 0.44 |
| Glu | 53 | A | A | . | . | . | . | . | −0.32 | . | * | . | −0.30 | 0.27 |
| Leu | 54 | A | A | . | . | . | . | . | −0.79 | . | * | . | −0.30 | 0.37 |
| Ala | 55 | A | A | . | . | . | . | . | −0.79 | . | * | . | −0.60 | 0.31 |
| Leu | 56 | A | A | . | . | . | . | . | −0.79 | . | * | . | −0.60 | 0.24 |
| His | 57 | A | A | . | . | . | . | . | −1.14 | . | * | . | −0.60 | 0.29 |
| Leu | 58 | A | A | . | . | . | . | . | −1.49 | * | * | . | −0.60 | 0.25 |
| Ser | 59 | A | A | . | . | . | . | . | −0.63 | * | * | . | −0.60 | 0.30 |
| Ala | 60 | A | A | . | . | . | . | . | −0.39 | * | * | . | −0.30 | 0.44 |
| Phe | 61 | A | A | . | . | . | . | . | −0.28 | * | * | . | −0.30 | 0.53 |
| Gly | 62 | . | . | . | . | T | T | . | −1.10 | * | . | . | −0.50 | 0.34 |
| Lys | 63 | A | . | . | . | . | T | . | −1.10 | . | * | F | −0.05 | 0.25 |
| Gly | 64 | . | . | B | . | . | T | . | −0.69 | . | * | . | −0.20 | 0.24 |
| Phe | 65 | . | . | B | . | . | T | . | −0.91 | . | * | . | 0.70 | 0.47 |
| Val | 66 | . | . | B | B | . | . | . | −0.80 | * | * | . | −0.30 | 0.19 |
| Leu | 67 | . | . | B | B | . | . | . | −0.67 | * | * | . | −0.30 | 0.20 |
| Arg | 68 | . | . | B | B | . | . | . | −0.71 | . | * | . | 0.00 | 0.35 |
| Leu | 69 | . | . | B | B | . | . | . | −0.37 | * | * | . | 1.20 | 0.80 |
| Ala | 70 | . | . | . | . | . | T | C | 0.03 | . | * | . | 2.55 | 1.61 |
| Pro | 71 | . | . | . | . | . | T | C | 0.19 | * | * | F | 3.00 | 1.10 |
| Asp | 72 | . | . | . | . | T | T | . | 0.19 | . | * | F | 2.60 | 1.16 |
| Asp | 73 | A | . | . | . | . | T | . | −0.51 | . | * | F | 1.75 | 0.95 |
| Ser | 74 | A | A | . | . | . | . | . | 0.09 | . | . | . | 0.90 | 0.62 |
| Phe | 75 | A | A | . | . | . | . | . | 0.68 | . | . | . | 0.60 | 0.57 |
| Leu | 76 | A | A | . | . | . | . | . | 0.19 | . | * | . | 0.30 | 0.59 |
| Ala | 77 | A | A | . | . | . | . | . | 0.23 | . | * | . | −0.60 | 0.38 |
| Pro | 78 | A | A | . | . | . | . | . | −0.66 | . | * | . | −0.30 | 0.89 |
| Glu | 79 | A | A | . | . | . | . | . | −0.36 | * | * | F | −0.15 | 0.75 |
| Phe | 80 | A | A | . | . | . | . | . | 0.46 | * | . | F | 0.90 | 1.29 |
| Lys | 81 | A | A | . | . | . | . | . | 0.46 | * | * | F | 0.90 | 1.63 |
| Ile | 82 | A | A | . | . | . | . | . | 0.70 | * | . | F | 0.75 | 0.78 |
| Glu | 83 | A | A | . | . | . | . | . | 0.57 | * | * | F | 0.45 | 0.89 |
| Arg | 84 | A | A | . | . | . | . | . | 0.27 | * | * | F | 0.75 | 0.44 |
| Leu | 85 | . | A | . | . | T | . | . | 0.62 | * | * | F | 0.85 | 0.84 |
| Gly | 86 | . | A | . | . | T | . | . | 0.69 | * | * | F | 1.15 | 0.48 |
| Gly | 87 | . | . | . | . | . | T | C | 0.99 | * | * | F | 1.35 | 0.48 |
| Ser | 88 | . | . | . | . | . | T | C | 0.68 | * | * | F | 1.05 | 0.59 |
| Gly | 89 | . | . | . | . | . | T | C | 0.22 | * | * | F | 1.05 | 0.86 |
| Arg | 90 | . | . | B | . | . | T | . | 0.69 | . | * | F | 1.19 | 0.86 |
| Ala | 91 | . | . | . | . | . | T | C | 1.03 | . | * | F | 1.73 | 0.63 |
| Thr | 92 | . | . | B | . | . | T | . | 1.49 | . | * | F | 2.32 | 1.11 |
| Gly | 93 | . | . | B | . | . | T | . | 1.44 | . | * | F | 2.66 | 1.11 |
| Gly | 94 | . | . | . | . | T | T | . | 0.98 | * | * | F | 3.40 | 1.09 |
| Glu | 95 | . | . | B | . | . | . | . | 0.98 | * | * | F | 2.31 | 0.62 |
| Arg | 96 | . | . | B | . | . | . | . | 1.22 | * | . | F | 2.12 | 1.23 |
| Gly | 97 | . | . | . | . | T | . | . | 0.87 | * | * | F | 2.18 | 1.23 |
| Leu | 98 | . | . | B | . | . | T | . | 0.51 | * | . | F | 1.49 | 0.38 |
| Arg | 99 | . | . | B | . | . | T | . | 0.16 | * | . | . | 0.70 | 0.17 |
| Gly | 100 | . | . | B | . | . | T | . | −0.14 | * | . | . | −0.20 | 0.15 |
| Cys | 101 | . | . | B | . | . | T | . | −0.60 | * | . | . | −0.20 | 0.24 |
| Phe | 102 | . | . | B | . | . | . | . | −0.57 | . | * | . | −0.10 | 0.12 |
| Phe | 103 | . | . | B | . | . | T | . | −0.61 | . | * | . | −0.20 | 0.18 |
| Ser | 104 | . | . | B | . | . | T | . | −0.72 | . | * | F | −0.05 | 0.24 |
| Gly | 105 | . | . | . | . | . | T | C | −0.72 | . | * | F | 0.15 | 0.45 |
| Thr | 106 | . | . | . | . | . | T | C | −0.06 | * | * | F | 0.45 | 0.52 |
| Val | 107 | . | . | . | B | . | . | C | 0.43 | . | * | F | 1.25 | 0.67 |
| Asn | 108 | . | . | . | B | . | . | C | 1.13 | . | * | F | 1.70 | 1.05 |
| Gly | 109 | . | . | . | B | . | . | C | 1.13 | . | * | F | 2.30 | 1.26 |
| Glu | 110 | . | . | . | . | . | T | C | 0.67 | . | * | F | 3.00 | 2.27 |
| Pro | 111 | A | . | . | . | . | T | . | 0.39 | . | * | F | 2.50 | 1.16 |
| Glu | 112 | A | . | . | . | . | T | . | 0.66 | . | * | F | 2.20 | 1.19 |
| Ser | 113 | A | . | . | . | . | T | . | −0.20 | . | . | F | 1.75 | 0.69 |
| Leu | 114 | A | A | . | B | . | . | . | −0.16 | . | . | . | 0.00 | 0.33 |
| Ala | 115 | A | A | . | B | . | . | . | −0.97 | . | . | . | −0.30 | 0.26 |
| Ala | 116 | A | A | . | B | . | . | . | −1.42 | . | . | . | −0.60 | 0.16 |
| Val | 117 | A | A | . | B | . | . | . | −1.31 | . | . | . | −0.60 | 0.10 |
| Ser | 118 | . | A | B | B | . | . | . | −1.36 | * | . | . | −0.30 | 0.20 |
| Leu | 119 | . | . | B | B | . | . | . | −1.36 | * | . | . | −0.30 | 0.20 |
| Cys | 120 | . | . | B | . | . | T | . | −1.07 | * | . | . | 0.10 | 0.22 |
| Arg | 121 | . | . | B | . | . | T | . | −0.82 | * | . | . | 0.10 | 0.22 |
| Gly | 122 | . | . | . | . | T | T | . | −0.27 | * | . | F | 0.65 | 0.26 |
| Leu | 123 | . | . | . | . | T | T | . | −0.67 | * | . | F | 1.25 | 0.65 |
| Ser | 124 | . | . | . | . | . | T | C | −0.67 | * | . | F | 0.45 | 0.29 |
| Gly | 125 | . | . | B | . | . | T | . | −0.81 | . | * | F | −0.05 | 0.24 |
| Ser | 126 | . | . | B | . | . | T | . | −0.92 | . | * | F | −0.05 | 0.24 |
| Phe | 127 | . | . | B | . | . | T | . | −0.92 | . | * | . | 0.10 | 0.30 |
| Leu | 128 | . | A | B | . | . | . | C | −0.11 | . | * | . | −0.30 | 0.30 |
| Leu | 129 | . | A | . | . | . | . | . | 0.19 | . | * | F | 0.65 | 0.39 |
| Asp | 130 | A | A | . | . | . | . | . | −0.17 | . | . | F | 0.45 | 0.77 |
| Gly | 131 | A | A | . | . | . | . | . | −0.18 | . | . | F | 0.45 | 0.81 |
| Glu | 132 | A | A | . | . | . | . | . | −0.37 | . | * | F | 0.90 | 1.42 |
| Glu | 133 | A | A | . | . | . | . | . | 0.44 | . | * | F | 0.75 | 0.60 |
| Phe | 134 | A | A | . | . | . | . | . | 1.04 | . | * | . | 0.45 | 1.04 |
| Thr | 135 | . | A | B | . | . | . | . | 1.04 | . | * | . | 0.30 | 0.93 |
| Ile | 136 | . | . | B | . | . | . | . | 1.04 | . | * | F | 0.05 | 0.93 |
| Gln | 137 | . | . | B | . | . | . | . | 0.46 | . | * | F | −0.10 | 1.06 |
| Pro | 138 | . | . | . | . | . | . | C | 0.11 | . | * | F | 0.25 | 0.75 |
| Gln | 139 | . | . | . | . | T | . | . | 0.47 | . | * | F | 0.60 | 1.05 |
| Gly | 140 | . | . | . | . | . | T | C | 0.48 | . | * | F | 0.45 | 0.60 |
| Ala | 141 | . | . | . | . | T | T | . | 0.56 | . | * | F | 1.25 | 0.52 |
| Gly | 142 | . | . | . | . | . | T | C | −0.03 | . | . | F | 0.45 | 0.25 |
| Gly | 143 | . | . | . | . | . | T | C | 0.18 | . | . | F | 0.65 | 0.25 |
| Ser | 144 | . | . | . | . | . | . | C | −0.03 | . | . | F | 0.65 | 0.43 |
| Leu | 145 | . | . | B | . | . | . | . | 0.28 | * | . | F | 0.65 | 0.68 |
| Ala | 146 | . | . | B | . | . | . | . | 0.98 | * | . | F | 0.85 | 0.93 |
| Gln | 147 | . | . | B | . | . | T | . | 0.51 | . | * | F | 2.00 | 1.36 |
| Pro | 148 | . | . | B | . | . | T | . | 0.86 | * | . | . | 1.05 | 1.36 |
| His | 149 | . | . | B | . | . | T | . | 1.27 | * | . | . | 1.45 | 2.34 |
| Arg | 150 | . | . | B | . | . | T | . | 1.79 | * | . | . | 1.55 | 2.64 |
| Leu | 151 | . | . | B | . | . | . | . | 2.03 | * | . | . | 0.85 | 1.80 |
| Gln | 152 | . | . | B | . | . | . | . | 1.82 | * | . | . | 0.65 | 1.31 |
| Arg | 153 | . | . | . | . | T | . | . | 1.44 | * | . | . | 1.05 | 1.03 |
| Trp | 154 | . | . | . | . | T | . | . | 1.13 | * | . | F | 0.84 | 1.26 |
| Gly | 155 | . | . | . | . | . | T | C | 0.43 | * | . | F | 0.93 | 0.72 |
| Pro | 156 | . | . | . | . | . | T | C | 1.36 | * | . | F | 1.17 | 0.37 |
| Ala | 157 | . | . | . | . | T | T | . | 1.14 | * | . | F | 1.61 | 0.69 |
| Gly | 158 | . | . | . | . | . | T | C | 0.22 | * | . | F | 2.40 | 1.08 |
| Ala | 159 | . | . | . | . | . | . | C | 0.30 | * | * | F | 1.81 | 0.58 |
| Arg | 160 | . | . | B | . | . | . | . | 0.76 | * | * | F | 1.37 | 0.89 |
| Pro | 161 | . | . | B | . | . | . | . | 0.62 | * | * | F | 1.58 | 1.75 |
| Leu | 162 | . | . | . | . | . | . | C | 1.00 | * | * | F | 1.84 | 1.72 |
| Pro | 163 | . | . | . | . | . | . | C | 1.34 | * | * | F | 1.90 | 1.35 |
| Arg | 164 | . | . | . | . | . | . | C | 1.64 | * | * | F | 2.20 | 1.52 |
| Gly | 165 | . | . | . | . | . | T | C | 1.53 | * | * | F | 2.40 | 1.93 |
| Pro | 166 | . | . | . | . | . | T | C | 0.89 | * | . | F | 3.00 | 2.17 |
| Glu | 167 | . | . | . | . | . | T | C | 1.70 | * | . | F | 2.55 | 0.82 |
| Trp | 168 | A | . | . | . | . | T | . | 1.60 | * | . | . | 2.05 | 1.44 |
| Glu | 169 | A | . | . | . | . | . | . | 1.14 | * | . | . | 1.85 | 1.34 |
| Val | 170 | A | . | . | . | . | . | . | 1.49 | | . | F | 1.85 | 0.77 |
| Glu | 171 | A | . | . | . | . | . | . | 1.36 | | * | F | 2.00 | 1.26 |
| Thr | 172 | A | . | . | . | . | . | . | 1.36 | | * | F | 2.15 | 0.72 |
| Gly | 173 | . | . | . | . | . | T | C | 1.76 | | . | F | 3.00 | 1.68 |
| Glu | 174 | A | . | . | . | . | T | . | 1.76 | | . | F | 2.50 | 1.90 |
| Gly | 175 | A | . | . | . | . | T | . | 2.61 | | . | F | 2.20 | 2.28 |
| Gln | 176 | A | . | . | . | . | T | . | 2.72 | | . | F | 1.90 | 4.00 |
| Arg | 177 | A | A | . | . | . | . | . | 2.69 | | * | F | 1.54 | 4.52 |
| Gln | 178 | A | A | . | . | . | . | . | 3.03 | | * | F | 1.58 | 4.52 |
| Glu | 179 | . | A | . | . | T | . | . | 3.00 | * | * | F | 2.32 | 4.36 |
| Arg | 180 | . | A | . | . | T | . | . | 3.34 | * | . | F | 2.66 | 3.03 |
| Gly | 181 | . | . | . | . | T | T | . | 3.34 | . | * | F | 3.40 | 3.03 |
| Asp | 182 | . | . | . | . | . | T | C | 3.23 | . | . | F | 2.86 | 3.03 |
| His | 183 | . | . | . | . | . | T | C | 2.93 | . | * | F | 2.52 | 2.58 |
| Gln | 184 | . | . | . | . | . | T | C | 2.93 | . | * | F | 2.18 | 3.50 |
| Glu | 185 | . | A | . | . | . | . | C | 2.82 | . | * | F | 1.44 | 3.63 |
| Asp | 186 | A | A | . | . | . | . | . | 3.17 | . | . | F | 0.90 | 4.61 |
| Ser | 187 | A | A | . | . | . | . | . | 2.87 | . | . | F | 0.90 | 4.61 |
| Glu | 188 | A | A | . | . | . | . | . | 2.90 | . | . | F | 0.90 | 3.57 |
| Glu | 189 | A | A | . | . | . | . | . | 2.90 | . | . | F | 0.90 | 3.70 |
| Glu | 190 | A | A | . | . | . | . | . | 2.90 | . | . | F | 0.90 | 4.79 |
| Ser | 191 | A | A | . | . | . | . | . | 2.90 | . | . | F | 0.90 | 4.79 |
| Gln | 192 | A | A | . | . | . | . | . | 2.61 | . | . | F | 0.90 | 4.79 |
| Glu | 193 | A | A | . | . | . | . | . | 2.61 | . | . | F | 0.90 | 2.79 |
| Glu | 194 | A | A | . | . | . | . | . | 2.27 | . | . | F | 0.90 | 3.61 |
| Glu | 195 | A | A | . | . | . | . | . | 1.68 | . | . | F | 0.90 | 2.06 |
| Ala | 196 | A | A | . | . | . | . | . | 1.68 | . | . | F | 1.16 | 1.20 |
| Glu | 197 | A | A | . | . | . | . | . | 1.68 | . | . | F | 1.27 | 0.93 |
| Gly | 198 | A | A | . | . | . | . | . | 1.47 | . | . | F | 1.53 | 0.93 |
| Ala | 199 | . | A | . | . | T | . | . | 1.26 | . | . | F | 2.34 | 1.42 |
| Ser | 200 | . | . | . | . | . | . | C | 1.04 | . | . | F | 2.60 | 1.27 |
| Glu | 201 | . | . | . | . | . | . | C | 1.42 | * | . | F | 2.04 | 1.99 |
| Pro | 202 | . | . | . | . | . | . | C | 0.61 | * | . | F | 1.78 | 3.04 |
| Pro | 203 | . | . | . | . | . | . | C | 0.61 | . | . | F | 1.52 | 1.87 |
| Pro | 204 | . | . | . | . | . | T | C | 0.61 | . | . | F | 1.46 | 1.07 |
| Pro | 205 | . | . | . | . | . | T | C | 0.60 | . | . | F | 0.45 | 0.70 |
| Leu | 206 | . | . | . | . | . | T | C | 0.30 | * | * | F | 0.45 | 0.65 |
| Gly | 207 | . | . | B | . | . | T | . | 0.62 | . | * | F | 0.51 | 0.57 |
| Ala | 208 | . | . | B | . | . | . | . | 0.52 | . | * | F | 1.17 | 0.72 |
| Thr | 209 | . | . | B | . | . | . | . | 0.78 | * | * | F | 1.58 | 1.25 |
| Ser | 210 | . | . | B | . | . | T | . | 1.10 | * | . | F | 2.34 | 2.53 |
| Arg | 211 | . | . | B | . | . | T | . | 1.21 | * | . | F | 2.60 | 4.91 |
| Thr | 212 | . | . | B | . | . | T | . | 0.70 | * | . | F | 2.34 | 2.95 |
| Lys | 213 | . | . | B | . | . | T | . | 0.99 | * | . | F | 2.08 | 1.63 |
| Arg | 214 | . | . | B | B | . | . | . | 1.30 | * | . | F | 1.42 | 1.12 |
| Phe | 215 | . | . | B | B | . | . | . | 1.01 | * | * | . | 1.01 | 1.34 |
| Val | 216 | . | . | B | B | . | . | . | 1.01 | * | * | . | 0.60 | 0.68 |
| Ser | 217 | A | . | . | B | . | . | . | 0.62 | * | * | . | 0.60 | 0.68 |
| Glu | 218 | A | A | . | . | . | . | . | −0.28 | * | * | . | −0.30 | 0.68 |
| Ala | 219 | A | A | . | B | . | . | . | −0.39 | * | * | . | 0.30 | 0.68 |
| Arg | 220 | A | A | . | B | . | . | . | 0.00 | * | * | . | 0.60 | 0.87 |
| Phe | 221 | A | A | . | B | . | . | . | 0.04 | * | . | . | 0.60 | 0.73 |
| Val | 222 | A | A | . | B | . | . | . | −0.47 | * | * | . | −0.30 | 0.59 |
| Glu | 223 | A | A | . | B | . | . | . | −1.32 | * | * | . | −0.30 | 0.25 |
| Thr | 224 | A | A | . | B | . | . | . | −1.32 | * | * | . | −0.60 | 0.21 |
| Leu | 225 | A | A | . | B | . | . | . | −1.43 | * | * | . | −0.60 | 0.29 |
| Leu | 226 | A | A | . | B | . | . | . | −1.32 | . | . | . | 0.30 | 0.28 |
| Val | 227 | A | A | . | B | . | . | . | −0.77 | . | . | . | −0.60 | 0.20 |
| Ala | 228 | A | A | . | B | . | . | . | −1.37 | . | . | . | −0.30 | 0.32 |
| Asp | 229 | A | A | . | B | . | . | . | −1.64 | . | . | . | −0.30 | 0.38 |
| Ala | 230 | A | A | . | . | . | . | . | −1.42 | . | . | . | −0.30 | 0.52 |
| Ser | 231 | A | A | . | . | . | . | . | −1.31 | . | . | . | 0.30 | 0.52 |
| Met | 232 | A | A | . | . | . | . | . | −0.70 | . | . | . | −0.30 | 0.27 |
| Ala | 233 | A | A | . | . | . | . | . | −0.46 | . | . | . | −0.60 | 0.42 |
| Ala | 234 | A | A | . | . | . | . | . | −1.04 | . | . | . | −0.60 | 0.31 |
| Phe | 235 | A | A | . | . | . | . | . | −0.46 | . | . | . | −0.60 | 0.32 |
| Tyr | 236 | A | A | . | . | . | . | . | −0.97 | . | . | . | −0.60 | 0.52 |
| Gly | 237 | A | A | . | . | . | . | . | −0.37 | . | . | . | −0.60 | 0.43 |
| Ala | 238 | A | A | . | . | . | . | . | 0.22 | . | . | . | −0.60 | 0.86 |
| Asp | 239 | A | A | . | . | . | . | . | 0.78 | * | * | . | −0.30 | 0.88 |
| Leu | 240 | A | A | . | . | . | . | . | 0.59 | * | . | . | 0.45 | 1.21 |
| Gln | 241 | A | A | . | B | . | . | . | 0.02 | * | * | . | −0.30 | 0.84 |
| Asn | 242 | A | A | . | B | . | . | . | 0.06 | * | . | . | −0.30 | 0.41 |
| His | 243 | . | A | B | B | . | . | . | −0.17 | * | * | . | −0.60 | 0.72 |
| Ile | 244 | . | A | B | B | . | . | . | −0.77 | * | . | . | −0.60 | 0.35 |
| Leu | 245 | . | A. | B | B | . | . | . | −0.26 | * | . | . | −0.60 | 0.21 |
| Thr | 246 | . | A | B | B | . | . | . | −1.11 | * | . | . | −0.60 | 0.21 |
| Leu | 247 | . | A | B | B | . | . | . | −1.70 | * | . | . | −0.60 | 0.22 |
| Met | 248 | A | A | . | B | . | . | . | −2.26 | * | * | . | −0.60 | 0.27 |
| Ser | 249 | A | A | . | B | . | . | . | −1.26 | * | * | . | −0.60 | 0.19 |
| Val | 250 | A | A | . | B | . | . | . | −1.33 | * | * | . | −0.30 | 0.45 |
| Ala | 251 | A | A | . | B | . | . | . | −1.27 | * | * | . | −0.30 | 0.32 |
| Ala | 252 | A | A | . | B | . | . | . | −0.41 | * | * | . | −0.60 | 0.37 |
| Arg | 253 | A | A | . | B | . | . | . | 0.16 | * | * | . | −0.15 | 1.01 |
| Ile | 254 | A | A | . | B | . | . | . | 0.24 | * | * | . | 0.45 | 1.36 |
| Tyr | 255 | A | . | . | . | . | . | . | 0.80 | * | * | . | 0.99 | 2.08 |
| Lys | 256 | . | . | B | . | . | . | . | 0.50 | * | * | . | 1.33 | 1.42 |
| His | 257 | . | . | B | . | . | T | . | 1.13 | . | * | F | 1.12 | 1.42 |
| Pro | 258 | . | . | . | . | | T | C | 1.02 | . | * | F | 2.56 | 1.81 |
| Ser | 259 | . | . | . | . | T | T | . | 1.61 | . | * | F | 3.40 | 1.46 |
| Ile | 260 | . | . | . | . | T | T | . | 0.97 | . | * | F | 2.76 | 1.44 |
| Lys | 261 | . | . | B | . | . | . | . | 0.92 | . | * | F | 1.67 | 0.65 |
| Asn | 262 | . | . | . | . | T | . | . | 0.14 | * | * | F | 1.73 | 0.78 |
| Ser | 263 | . | . | B | B | . | . | . | −0.24 | * | * | F | 0.19 | 0.92 |
| Ile | 264 | . | . | B | B | . | . | . | −0.80 | * | * | . | −0.30 | 0.45 |
| Asn | 265 | . | . | B | B | . | . | . | −0.77 | * | * | . | −0.60 | 0.21 |
| Leu | 266 | . | . | B | B | . | . | . | −0.77 | . | * | . | −0.60 | 0.12 |
| Met | 267 | A | . | . | B | . | . | . | −1.62 | * | . | . | −0.60 | 0.33 |
| Val | 268 | . | . | B | B | . | . | . | −2.13 | . | * | . | −0.60 | 0.15 |
| Val | 269 | . | . | B | B | . | . | . | −2.13 | . | . | . | −0.60 | 0.15 |
| Lys | 270 | A | . | . | B | . | . | . | −2.99 | . | . | . | −0.60 | 0.11 |
| Val | 271 | . | . | B | B | . | . | . | −2.18 | . | . | . | −0.60 | 0.11 |
| Leu | 272 | . | . | B | B | . | . | . | −1.58 | . | . | . | −0.30 | 0.25 |
| Ile | 273 | A | . | . | B | . | . | . | −0.72 | . | . | . | 0.30 | 0.21 |
| Val | 274 | A | . | . | B | . | . | . | 0.18 | . | * | . | 0.30 | 0.49 |
| Glu | 275 | A | . | . | B | . | . | . | −0.16 | . | . | . | 0.75 | 1.19 |
| Asp | 276 | A | A | . | . | . | . | . | 0.36 | . | . | F | 0.90 | 1.79 |
| Glu | 277 | A | A | . | . | . | . | . | 0.96 | * | . | F | 0.90 | 2.39 |
| Lys | 278 | . | A | . | . | T | . | . | 1.84 | * | * | F | 1.30 | 2.13 |
| Trp | 279 | . | A | . | . | . | . | C | 1.84 | . | * | F | 1.10 | 2.21 |
| Gly | 280 | . | . | . | . | . | T | C | 1.54 | * | . | F | 1.35 | 0.95 |
| Pro | 281 | . | . | . | . | . | T | C | 1.54 | * | * | F | 1.36 | 0.64 |
| Glu | 282 | . | . | B | . | . | T | . | 1.54 | * | * | F | 1.62 | 1.01 |
| Val | 283 | . | . | B | . | . | T | . | 1.16 | * | * | F | 2.23 | 1.64 |
| Ser | 284 | . | . | . | . | . | T | C | 1.10 | . | * | F | 2.74 | 1.05 |
| Asp | 285 | . | . | . | . | T | T | . | 0.63 | . | . | F | 3.10 | 0.60 |
| Asn | 286 | . | . | . | . | T | T | . | 0.53 | . | * | F | 2.49 | 0.67 |
| Gly | 287 | . | . | . | . | T | T | . | −0.28 | * | * | F | 2.18 | 0.72 |
| Gly | 288 | . | . | . | . | T | . | . | 0.69 | * | * | F | 1.07 | 0.35 |
| Leu | 289 | . | . | B | . | . | . | . | 0.99 | * | * | F | 0.36 | 0.43 |
| Thr | 290 | . | . | B | . | . | . | . | 0.29 | * | * | . | −0.10 | 0.70 |
| Leu | 291 | . | . | B | . | . | . | . | −0.38 | * | * | . | −0.40 | 0.61 |
| Arg | 292 | . | . | B | . | . | . | . | −0.03 | * | * | . | −0.40 | 0.40 |
| Asn | 293 | . | . | B | . | . | . | . | 0.02 | * | * | . | −0.10 | 0.44 |
| Phe | 294 | . | . | . | . | T | T | . | 0.83 | * | * | . | 0.20 | 0.57 |
| Cys | 295 | . | . | . | . | T | T | . | 1.26 | * | * | . | 0.20 | 0.50 |
| Asn | 296 | . | . | . | . | T | T | . | 2.18 | * | * | . | 0.20 | 0.61 |
| Trp | 297 | . | . | . | . | T | T | . | 1.37 | * | * | . | 0.65 | 1.38 |
| Gln | 298 | . | . | . | . | T | . | . | 1.37 | * | . | . | 0.45 | 2.23 |
| Arg | 299 | . | . | . | . | T | . | . | 2.07 | * | . | . | 1.05 | 2.23 |
| Arg | 300 | . | . | . | . | T | . | . | 2.52 | * | * | F | 1.20 | 3.67 |
| Phe | 301 | . | . | . | . | T | . | . | 2.22 | * | * | F | 1.84 | 3.28 |
| Asn | 302 | . | . | . | . | T | . | . | 2.51 | * | * | F | 2.18 | 2.24 |
| Gln | 303 | . | . | . | . | . | T | C | 2.62 | * | . | F | 2.52 | 1.91 |
| Pro | 304 | . | . | . | . | . | T | C | 2.48 | * | . | F | 2.86 | 4.33 |
| Ser | 305 | . | . | . | . | T | T | . | 2.16 | * | * | F | 3.40 | 3.66 |
| Asp | 306 | . | . | . | . | T | T | . | 2.86 | * | . | F | 3.06 | 3.27 |
| Arg | 307 | . | . | . | . | . | . | C | 2.82 | * | . | F | 2.32 | 3.66 |
| His | 308 | . | . | . | . | . | . | C | 2.58 | * | . | F | 1.98 | 3.72 |
| Pro | 309 | . | . | . | . | . | . | C | 2.79 | * | . | F | 1.64 | 3.49 |
| Glu | 310 | . | . | . | . | T | . | . | 2.78 | * | . | F | 1.50 | 2.97 |
| His | 311 | A | . | . | . | . | T | . | 2.19 | * | . | F | 1.00 | 3.15 |
| Tyr | 312 | A | . | . | . | . | T | . | 1.19 | * | . | F | 1.00 | 2.06 |
| Asp | 313 | A | . | . | . | . | T | . | 0.41 | . | . | F | 0.85 | 0.83 |
| Thr | 314 | A | . | . | . | . | T | . | −0.19 | . | . | . | −0.20 | 0.51 |
| Ala | 315 | A | . | . | B | . | . | . | −0.50 | * | . | . | −0.60 | 0.27 |
| Ile | 316 | . | . | B | B | . | . | . | −0.36 | * | . | . | −0.60 | 0.23 |
| Leu | 317 | . | . | B | B | . | . | . | −0.11 | . | . | . | −0.60 | 0.31 |
| Leu | 318 | . | . | B | B | . | . | . | −0.11 | . | * | . | −0.60 | 0.53 |
| Thr | 319 | . | . | B | B | . | . | . | −0.50 | . | . | F | 0.00 | 1.23 |
| Arg | 320 | . | . | B | B | . | . | . | −0.58 | . | * | F | −0.08 | 1.29 |
| Gln | 321 | . | . | . | B | T | . | . | −0.03 | . | * | F | 0.69 | 0.84 |
| Asn | 322 | . | . | . | . | T | T | . | 0.78 | . | * | F | 1.31 | 0.57 |
| Phe | 323 | . | . | . | . | T | T | . | 1.59 | . | . | . | 1.98 | 0.51 |
| Cys | 324 | . | . | . | . | T | T | . | 1.56 | . | * | . | 2.20 | 0.51 |
| Gly | 325 | . | . | . | . | T | T | . | 0.63 | . | * | F | 1.53 | 0.31 |
| Gln | 326 | . | . | . | . | T | . | . | −0.03 | . | . | F | 1.11 | 0.30 |
| Glu | 327 | . | . | . | . | T | . | . | −0.03 | . | . | F | 0.89 | 0.30 |
| Gly | 328 | . | . | . | . | T | . | . | 0.36 | . | . | F | 1.27 | 0.50 |
| Leu | 329 | . | . | B | . | . | . | . | 0.21 | . | . | F | 0.65 | 0.42 |
| Cys | 330 | . | . | B | . | . | . | . | 0.21 | . | . | . | 0.50 | 0.20 |
| Asp | 331 | . | . | B | . | . | T | . | −0.64 | . | . | . | 0.10 | 0.20 |
| Thr | 332 | . | . | B | . | . | T | . | −1.23 | * | . | . | −0.20 | 0.18 |
| Leu | 333 | . | . | B | . | . | T | . | −0.89 | . | . | . | 0.10 | 0.34 |
| Gly | 334 | . | . | B | . | . | T | . | −0.97 | . | . | . | 0.70 | 0.34 |
| Val | 335 | . | . | B | . | . | . | . | −0.64 | . | . | . | −0.40 | 0.16 |
| Ala | 336 | . | . | B | . | . | . | . | −0.96 | . | . | . | −0.10 | 0.20 |
| Asp | 337 | . | . | B | . | . | T | . | −1.53 | . | . | . | 0.10 | 0.29 |
| Ile | 338 | . | . | B | . | . | T | . | −1.39 | . | . | . | −0.20 | 0.27 |
| Gly | 339 | . | . | B | . | . | T | . | −1.04 | * | . | . | 0.10 | 0.14 |
| Thr | 340 | . | . | B | . | . | T | . | −0.40 | . | . | . | 0.70 | 0.14 |
| Ile | 341 | . | . | B | . | . | . | . | 0.19 | . | . | . | 0.24 | 0.32 |
| Cys | 342 | . | . | B | . | . | . | . | 0.23 | . | . | . | 1.18 | 0.52 |
| Asp | 343 | . | . | B | . | . | T | . | 0.82 | * | . | F | 1.87 | 0.72 |
| Pro | 344 | . | . | . | . | T | T | . | 0.50 | . | . | F | 3.06 | 1.37 |
| Asn | 345 | . | . | . | . | T | T | . | 0.51 | . | . | F | 3.40 | 1.37 |
| Lys | 346 | . | . | . | . | T | T | . | 0.54 | * | . | F | 3.06 | 1.10 |
| Ser | 347 | . | . | . | B | T | . | . | 0.32 | . | . | F | 1.87 | 0.53 |
| Cys | 348 | . | . | B | B | . | . | . | 0.32 | * | . | . | 0.38 | 0.23 |
| Ser | 349 | . | . | B | B | . | . | . | 0.53 | * | . | . | 0.64 | 0.20 |
| Val | 350 | . | . | B | B | . | . | . | 0.53 | * | . | . | 0.30 | 0.25 |
| Ile | 351 | . | . | B | B | . | . | . | 0.14 | * | . | . | 0.60 | 0.80 |
| Glu | 352 | A | . | . | B | . | . | . | −0.37 | . | . | . | 0.60 | 0.59 |
| Asp | 353 | A | A | . | . | . | . | . | 0.30 | . | . | F | 0.75 | 0.66 |
| Glu | 354 | A | A | . | . | . | . | . | 0.01 | * | . | F | 0.90 | 1.62 |
| Gly | 355 | A | A | . | . | . | . | . | 0.28 | * | . | F | 0.75 | 0.95 |
| Leu | 356 | A | A | . | . | . | . | . | 1.13 | * | . | . | 0.30 | 0.57 |
| Gln | 357 | A | A | . | . | . | . | . | 0.82 | * | . | . | −0.30 | 0.45 |
| Ala | 358 | A | A | . | . | . | . | . | 0.01 | * | . | . | −0.60 | 0.66 |
| Ala | 359 | A | A | . | . | . | . | . | −0.58 | * | . | . | −0.60 | 0.66 |
| His | 360 | A | A | . | . | . | . | . | −0.27 | * | . | . | −0.60 | 0.38 |
| Thr | 361 | A | A | . | . | . | . | . | 0.54 | * | . | . | −0.60 | 0.52 |
| Leu | 362 | A | A | . | . | . | . | . | −0.27 | * | . | . | −0.30 | 0.88 |
| Ala | 363 | A | A | . | . | . | . | . | −0.02 | * | . | . | −0.30 | 0.54 |
| His | 364 | A | A | . | . | . | . | . | 0.53 | * | . | . | −0.30 | 0.37 |
| Glu | 365 | A | A | . | . | . | . | . | −0.29 | * | . | . | −0.30 | 0.61 |
| Leu | 366 | A | A | . | B | . | . | . | −0.79 | * | . | . | −0.30 | 0.45 |
| Gly | 367 | A | A | . | B | . | . | . | −0.28 | * | . | . | −0.60 | 0.27 |
| His | 368 | A | A | . | B | . | . | . | −0.29 | * | . | . | −0.30 | 0.21 |
| Val | 369 | A | A | . | B | . | . | . | −0.47 | * | . | . | −0.60 | 0.25 |
| Leu | 370 | . | A | B | B | . | . | . | −0.50 | * | . | . | −0.26 | 0.39 |
| Ser | 371 | . | A | B | B | . | . | . | 0.31 | * | . | . | 0.08 | 0.39 |
| Met | 372 | . | . | B | . | . | . | . | 0.66 | . | . | . | 0.92 | 0.88 |
| Pro | 373 | . | . | . | . | T | . | . | 0.39 | * | . | . | 2.41 | 1.78 |
| His | 374 | . | . | . | . | T | T | . | 1.29 | * | . | F | 3.40 | 1.78 |
| Asp | 375 | . | . | . | . | T | T | . | 1.89 | . | . | F | 3.06 | 3.61 |
| Asp | 376 | . | . | . | . | T | T | . | 1.52 | . | . | F | 2.89 | 3.61 |
| Ser | 377 | . | . | . | . | T | T | . | 1.81 | * | * | F | 2.72 | 1.42 |
| Lys | 378 | . | . | B | . | . | T | . | 2.13 | * | * | F | 2.15 | 1.23 |
| Pro | 379 | . | . | . | . | T | T | . | 1.36 | * | * | F | 2.38 | 1.44 |
| Cys | 380 | . | . | B | . | . | T | . | 0.66 | * | * | F | 1.70 | 0.89 |
| Thr | 381 | . | . | B | . | . | T | . | 0.31 | * | * | F | 1.53 | 0.38 |
| Arg | 382 | . | . | B | B | . | . | . | 0.40 | * | * | F | 0.36 | 0.25 |
| Leu | 383 | . | . | B | B | . | . | . | −0.24 | * | * | . | 0.04 | 0.71 |
| Phe | 384 | . | . | B | B | . | . | . | −0.38 | * | . | . | −0.43 | 0.49 |
| Gly | 385 | . | . | . | B | . | . | C | 0.33 | * | . | F | 0.05 | 0.25 |
| Pro | 386 | . | . | . | . | . | T | C | 0.61 | * | * | F | 0.45 | 0.59 |
| Met | 387 | . | . | . | . | T | T | . | 0.47 | * | * | F | 0.65 | 0.93 |
| Gly | 388 | A | . | . | . | . | T | . | 0.42 | . | . | F | 1.00 | 1.29 |
| Lys | 389 | A | . | . | . | . | T | . | 0.52 | . | . | . | 0.10 | 0.62 |
| His | 390 | A | A | . | . | . | . | . | 0.28 | . | . | . | −0.30 | 0.62 |
| His | 391 | A | A | . | . | . | . | . | 0.28 | . | * | . | −0.30 | 0.63 |
| Val | 392 | . | A | B | . | . | . | . | 0.07 | . | . | . | −0.30 | 0.49 |
| Met | 393 | A | A | . | . | . | . | . | −0.29 | . | * | . | −0.60 | 0.30 |
| Ala | 394 | A | A | . | . | . | . | . | −1.19 | . | * | . | −0.60 | 0.19 |
| Pro | 395 | A | A | . | . | . | . | . | −1.19 | . | * | . | −0.60 | 0.19 |
| Leu | 396 | A | A | . | . | . | . | . | −1.97 | . | * | . | −0.60 | 0.26 |
| Phe | 397 | A | A | . | . | . | . | . | −1.11 | * | * | . | −0.60 | 0.21 |
| Val | 398 | A | A | . | . | . | . | . | −0.51 | * | . | . | −0.60 | 0.22 |
| His | 399 | . | A | B | . | . | . | . | −0.23 | * | * | . | −0.60 | 0.46 |
| Leu | 400 | . | A | B | . | . | . | . | −0.83 | * | * | . | −0.60 | 0.77 |
| Asn | 401 | . | A | . | . | T | . | . | −0.23 | * | * | F | −0.05 | 0.85 |
| Gln | 402 | . | A | . | . | T | . | . | 0.18 | . | * | F | −0.05 | 0.97 |
| Thr | 403 | . | A | . | . | T | . | . | 0.73 | . | * | F | 0.10 | 1.24 |
| Leu | 404 | . | A | . | . | . | . | C | 0.56 | . | * | F | −0.10 | 1.03 |
| Pro | 405 | . | . | . | . | T | . | . | 0.70 | . | . | . | 0.00 | 0.92 |
| Trp | 406 | . | . | . | . | T | . | . | 0.40 | . | . | . | 0.00 | 0.34 |
| Ser | 407 | . | . | . | . | . | T | C | −0.19 | . | . | . | 0.00 | 0.55 |
| Pro | 408 | . | . | . | . | T | T | . | −0.48 | . | . | . | 0.20 | 0.36 |
| Cys | 409 | . | . | . | . | T | T | . | 0.09 | . | . | . | 0.20 | 0.34 |
| Ser | 410 | . | . | B | . | . | T | . | −0.51 | . | . | . | −0.20 | 0.40 |
| Ala | 411 | . | A | B | . | . | . | . | −0.53 | . | . | . | −0.60 | 0.21 |
| Met | 412 | . | A | B | . | . | . | . | −0.23 | . | . | . | −0.60 | 0.57 |
| Tyr | 413 | . | A | B | . | . | . | . | −0.83 | . | . | . | −0.60 | 0.74 |
| Leu | 414 | . | A | B | . | . | . | . | −0.98 | * | . | . | −0.60 | 0.60 |
| Thr | 415 | . | A | B | . | . | . | . | −0.68 | * | . | . | −0.60 | 0.50 |
| Glu | 416 | A | A | . | . | . | . | . | −0.43 | * | . | . | −0.30 | 0.54 |
| Leu | 417 | A | A | . | . | . | . | . | −0.18 | * | . | F | 0.76 | 0.64 |
| Leu | 418 | A | . | . | . | . | T | . | 0.03 | * | . | F | 1.47 | 0.44 |
| Asp | 419 | . | . | . | . | T | T | . | 0.50 | * | . | F | 2.18 | 0.35 |
| Gly | 420 | . | . | . | . | T | T | . | 0.81 | . | . | F | 1.89 | 0.42 |
| Gly | 421 | . | . | . | . | T | T | . | 0.14 | . | . | F | 3.10 | 0.84 |
| His | 422 | . | . | . | . | T | T | . | 0.14 | . | . | F | 2.79 | 0.27 |
| Gly | 423 | . | . | . | . | T | T | . | 0.14 | . | . | F | 1.58 | 0.23 |
| Asp | 424 | . | . | B | . | . | T | . | 0.14 | . | * | . | 0.72 | 0.19 |
| Cys | 425 | . | . | B | . | . | T | . | −0.10 | . | * | . | 1.01 | 0.23 |
| Leu | 426 | . | . | B | . | . | . | . | 0.03 | . | * | . | 0.50 | 0.24 |
| Leu | 427 | . | . | B | . | . | . | . | −0.28 | . | * | . | 0.50 | 0.22 |
| Asp | 428 | . | . | B | . | . | . | . | −0.52 | * | * | . | −0.10 | 0.40 |
| Ala | 429 | . | . | B | . | . | T | . | −1.11 | * | . | F | 0.25 | 0.49 |
| Pro | 430 | A | . | . | . | . | T | . | −1.26 | . | . | F | 0.25 | 0.60 |
| Gly | 431 | . | . | . | . | T | T | . | −0.66 | . | . | F | 0.65 | 0.30 |
| Ala | 432 | . | . | B | . | . | T | . | −0.66 | . | . | . | −0.20 | 0.46 |
| Ala | 433 | . | . | B | . | . | . | . | −0.87 | . | . | . | −0.40 | 0.24 |
| Leu | 434 | . | . | B | . | . | . | . | −0.59 | . | . | . | −0.40 | 0.38 |
| Pro | 435 | . | . | B | . | . | . | . | −0.72 | . | . | . | −0.40 | 0.54 |
| Leu | 436 | . | . | B | . | . | T | . | −1.19 | . | . | . | −0.20 | 0.53 |
| Pro | 437 | . | . | B | . | . | T | . | −0.81 | . | . | F | 0.00 | 0.53 |
| Thr | 438 | . | . | . | . | T | T | . | −0.57 | . | * | F | 0.45 | 0.53 |
| Gly | 439 | . | . | . | . | . | T | C | 0.36 | . | * | F | 0.30 | 0.64 |
| Leu | 440 | . | . | . | . | . | T | C | −0.03 | . | * | F | 1.25 | 0.81 |
| Pro | 441 | . | . | B | . | . | T | . | 0.19 | . | * | F | 0.50 | 0.55 |
| Gly | 442 | . | . | B | . | . | T | . | −0.41 | . | * | F | 0.45 | 0.57 |
| Arg | 443 | . | . | B | . | . | T | . | −0.34 | . | * | . | 0.25 | 0.57 |
| Met | 444 | . | A | B | . | . | . | . | 0.00 | . | * | . | −0.50 | 0.57 |
| Ala | 445 | . | A | B | . | . | . | . | 0.00 | * | * | . | −0.10 | 1.00 |
| Leu | 446 | . | A | B | . | . | . | . | 0.21 | * | . | . | −0.60 | 0.42 |
| Tyr | 447 | . | A | B | . | . | . | . | 0.56 | * | * | . | −0.60 | 0.71 |
| Gln | 448 | . | A | B | . | . | . | . | 0.44 | * | * | . | −0.45 | 1.22 |
| Leu | 449 | A | A | . | . | . | . | . | 0.38 | * | * | . | −0.15 | 2.57 |
| Asp | 450 | A | A | . | . | . | . | . | 1.08 | * | * | F | −0.15 | 0.88 |
| Gln | 451 | . | A | B | . | . | . | . | 1.89 | * | * | F | 0.75 | 0.99 |
| Gln | 452 | . | A | B | . | . | . | . | 1.24 | * | * | F | 0.90 | 2.09 |
| Cys | 453 | . | A | B | . | . | . | . | 0.54 | * | * | F | 0.75 | 0.88 |
| Arg | 454 | . | A | B | . | . | . | . | 1.01 | * | * | . | −0.30 | 0.44 |
| Gln | 455 | . | A | B | . | . | . | . | 0.80 | * | * | . | −0.30 | 0.25 |
| Ile | 456 | . | A | B | . | . | . | . | 0.80 | * | . | . | −0.30 | 0.72 |
| Phe | 457 | . | A | . | . | T | . | . | 0.10 | * | * | . | 0.70 | 0.62 |
| Gly | 458 | . | . | . | . | . | T | C | 0.88 | * | * | . | 0.00 | 0.31 |
| Pro | 459 | . | . | . | . | T | T | . | 0.73 | * | * | F | 0.65 | 0.86 |
| Asp | 460 | . | . | . | . | T | T | . | 0.07 | * | * | F | 1.40 | 1.35 |
| Phe | 461 | . | . | . | . | T | T | . | 0.74 | * | * | . | 1.35 | 0.73 |
| Arg | 462 | . | . | . | . | T | . | . | 1.44 | * | * | . | 1.40 | 0.73 |
| His | 463 | . | . | . | . | T | . | . | 1.48 | * | * | . | 1.65 | 0.71 |
| Cys | 464 | . | . | . | . | . | T | C | 1.39 | * | * | . | 1.45 | 1.18 |
| Pro | 465 | . | . | . | . | T | T | . | 0.80 | * | . | F | 2.50 | 0.80 |
| Asn | 466 | . | . | . | . | T | T | . | 1.50 | * | * | F | 1.65 | 0.60 |
| Thr | 467 | . | . | . | . | T | T | . | 1.39 | * | * | F | 1.55 | 1.93 |
| Ser | 468 | . | A | . | . | T | . | . | 0.57 | * | . | F | 1.50 | 2.08 |
| Ala | 469 | . | A | . | . | T | . | . | 0.57 | . | . | F | 1.10 | 0.96 |
| Gln | 470 | . | A | B | . | . | . | . | 0.19 | . | . | F | 0.45 | 0.36 |
| Asp | 471 | . | A | B | . | . | . | . | 0.19 | * | * | F | 0.45 | 0.27 |
| Val | 472 | . | A | B | . | . | . | . | −0.31 | * | . | . | −0.30 | 0.46 |
| Cys | 473 | . | A | B | . | . | . | . | −0.30 | * | . | . | −0.30 | 0.22 |
| Ala | 474 | . | A | B | . | . | . | . | −0.38 | * | * | . | −0.60 | 0.14 |
| Gln | 475 | . | A | B | . | . | . | . | −0.41 | . | * | . | −0.60 | 0.10 |
| Leu | 476 | . | A | B | . | . | . | . | −0.72 | * | * | . | −0.60 | 0.25 |
| Trp | 477 | . | A | B | . | . | . | . | 0.13 | . | * | . | −0.60 | 0.36 |
| Cys | 478 | . | A | B | . | . | . | . | 0.46 | . | . | . | −0.26 | 0.35 |
| His | 479 | . | . | . | . | T | T | . | 0.46 | . | . | . | 0.88 | 0.42 |
| Thr | 480 | . | . | . | . | T | T | . | 0.46 | . | * | . | 1.52 | 0.40 |
| Asp | 481 | . | . | . | . | T | T | . | 1.06 | . | . | F | 3.06 | 1.30 |
| Gly | 482 | . | . | . | . | T | T | . | 0.53 | . | . | F | 3.40 | 1.48 |
| Ala | 483 | . | . | . | . | T | . | C | 0.53 | * | . | F | 2.41 | 0.85 |
| Glu | 484 | A | . | . | . | . | . | . | 0.53 | * | . | F | 1.67 | 0.27 |
| Pro | 485 | A | . | . | . | . | . | . | 0.53 | . | . | F | 0.73 | 0.37 |
| Leu | 486 | A | . | . | . | . | . | . | 0.58 | * | . | . | 0.24 | 0.53 |
| Cys | 487 | A | . | . | . | . | . | . | 0.92 | . | . | . | 0.78 | 0.62 |
| His | 488 | . | . | B | . | . | . | . | 1.17 | . | . | F | 0.61 | 0.64 |
| Thr | 489 | . | . | . | . | T | T | . | 0.87 | . | . | F | 1.49 | 0.77 |
| Lys | 490 | . | . | . | . | T | T | . | 0.27 | . | . | F | 2.52 | 1.92 |
| Asn | 491 | . | . | . | . | T | T | . | 0.87 | . | . | F | 2.80 | 1.16 |
| Gly | 492 | . | . | . | . | T | T | . | 1.24 | . | . | F | 2.52 | 1.25 |
| Ser | 493 | . | . | . | . | . | . | C | 0.69 | . | . | F | 1.09 | 0.66 |
| Leu | 494 | . | . | . | . | . | . | C | 1.00 | . | . | . | 0.36 | 0.41 |
| Pro | 495 | . | . | B | . | . | . | . | 0.61 | . | . | . | 0.18 | 0.69 |
| Trp | 496 | . | . | . | . | T | T | . | 0.30 | . | . | . | 0.50 | 0.51 |
| Ala | 497 | . | . | B | . | . | T | . | 0.43 | . | . | . | 0.05 | 0.90 |
| Asp | 498 | . | . | . | . | T | T | . | 0.07 | . | . | F | 1.15 | 0.90 |
| Gly | 499 | . | . | . | . | T | T | . | 0.53 | . | . | F | 1.40 | 0.46 |
| Thr | 500 | . | . | . | . | . | T | C | 0.53 | . | . | F | 2.05 | 0.45 |
| Pro | 501 | . | . | . | . | T | T | . | 0.48 | . | . | F | 2.50 | 0.42 |
| Cys | 502 | . | . | . | . | T | T | . | 1.03 | . | * | F | 1.65 | 0.42 |
| Gly | 503 | . | . | . | . | . | T | C | 0.22 | . | . | F | 1.20 | 0.39 |
| Pro | 504 | . | . | . | . | T | . | . | −0.10 | . | . | F | 0.65 | 0.21 |
| Gly | 505 | . | . | . | . | T | . | . | −0.09 | . | . | . | 0.25 | 0.21 |
| His | 506 | . | . | B | . | . | . | . | 0.12 | . | . | . | −0.40 | 0.28 |
| Leu | 507 | . | . | B | . | . | . | . | 0.44 | . | . | . | 0.50 | 0.32 |
| Cys | 508 | . | . | B | . | . | T | . | 0.49 | . | * | . | 0.91 | 0.32 |
| Ser | 509 | . | . | . | . | T | T | . | 0.03 | . | . | F | 1.67 | 0.31 |
| Glu | 510 | . | . | . | . | T | T | . | −0.43 | . | . | F | 1.28 | 0.20 |
| Gly | 511 | . | . | . | . | T | T | . | −0.61 | * | . | F | 1.49 | 0.31 |
| Ser | 512 | . | . | . | . | T | . | . | 0.20 | * | . | F | 2.10 | 0.36 |
| Cys | 513 | . | A | . | . | . | . | C | 0.87 | . | . | F | 1.79 | 0.36 |
| Leu | 514 | . | A | . | . | . | . | C | 1.17 | . | . | F | 1.58 | 0.63 |
| Pro | 515 | A | A | . | . | . | . | . | 0.31 | . | . | F | 1.17 | 0.81 |
| Glu | 516 | A | A | . | . | . | . | . | 0.66 | * | . | F | 1.11 | 1.13 |
| Glu | 517 | A | A | . | . | . | . | . | 1.07 | * | . | F | 0.90 | 2.37 |
| Glu | 518 | A | A | . | . | . | . | . | 1.52 | . | . | F | 0.90 | 3.00 |
| Val | 519 | A | A | . | . | . | . | . | 2.38 | . | . | F | 0.90 | 2.68 |
| Glu | 520 | A | A | . | . | . | . | . | 2.38 | * | . | F | 0.90 | 3.09 |
| Arg | 521 | A | . | . | . | . | T | . | 1.52 | * | . | F | 1.30 | 2.76 |
| Pro | 522 | A | . | . | . | . | T | . | 0.67 | * | * | F | 1.30 | 2.76 |
| Lys | 523 | A | . | . | . | . | T | . | 0.67 | * | * | F | 1.30 | 1.18 |
| Pro | 524 | . | . | B | . | . | T | . | 1.18 | * | * | F | 1.30 | 1.01 |
| Val | 525 | . | . | B | . | . | . | . | 0.83 | * | * | F | 0.65 | 0.65 |
| Val | 526 | . | . | B | . | . | . | . | 0.43 | . | * | F | 0.65 | 0.32 |
| Asp | 527 | . | . | B | . | . | T | . | 0.06 | * | . | F | −0.05 | 0.22 |
| Gly | 528 | . | . | B | . | . | T | . | −0.20 | * | . | F | −0.05 | 0.30 |
| Gly | 529 | . | . | . | . | T | T | . | −0.28 | . | . | F | 0.65 | 0.62 |
| Trp | 530 | . | . | . | . | . | T | C | 0.23 | . | . | . | 0.00 | 0.39 |
| Ala | 531 | . | . | . | . | . | . | C | 0.88 | . | . | . | −0.20 | 0.39 |
| Pro | 532 | . | . | . | . | T | . | . | 0.59 | . | . | . | 0.00 | 0.61 |
| Trp | 533 | . | . | . | . | T | . | . | 0.59 | . | . | . | 0.00 | 0.61 |
| Gly | 534 | . | . | . | . | . | T | C | 0.93 | . | . | . | 0.00 | 0.59 |
| Pro | 535 | . | . | . | . | T | T | . | 0.56 | . | . | F | 0.35 | 0.66 |
| Trp | 536 | . | . | . | . | T | T | . | 0.84 | * | . | F | 0.66 | 0.34 |
| Gly | 537 | . | . | . | . | . | T | C | 1.17 | * | . | F | 1.07 | 0.46 |
| Glu | 538 | . | . | . | . | T | . | . | 1.14 | * | . | F | 1.98 | 0.58 |
| Cys | 539 | . | . | . | . | T | T | . | 0.82 | * | . | F | 2.49 | 0.80 |
| Ser | 540 | . | . | . | . | T | T | . | 0.69 | * | . | F | 3.10 | 0.43 |
| Arg | 541 | . | . | . | . | T | T | . | 0.63 | * | . | F | 2.79 | 0.25 |
| Thr | 542 | . | . | . | . | T | T | . | 0.63 | * | . | F | 2.18 | 0.46 |
| Cys | 543 | . | . | . | . | T | T | . | −0.22 | * | . | F | 1.87 | 0.34 |
| Gly | 544 | . | . | . | . | T | T | . | 0.44 | * | . | F | 1.56 | 0.13 |
| Gly | 545 | . | . | . | . | T | T | . | 0.04 | * | * | F | 0.65 | 0.15 |
| Gly | 546 | . | . | . | . | T | T | . | −0.37 | * | * | F | 0.35 | 0.25 |
| Val | 547 | . | . | B | B | . | . | . | −0.09 | * | * | . | −0.60 | 0.33 |
| Gln | 548 | . | . | B | B | . | . | . | 0.69 | * | * | . | −0.60 | 0.46 |
| Phe | 549 | . | . | B | B | . | . | . | 1.03 | * | * | . | −0.30 | 0.91 |
| Ser | 550 | . | . | B | B | . | . | . | 0.71 | * | * | . | 0.79 | 2.13 |
| His | 551 | . | . | B | . | . | . | . | 1.10 | * | * | . | 1.18 | 0.66 |
| Arg | 552 | . | . | . | . | T | . | . | 1.96 | * | * | . | 2.37 | 1.52 |
| Glu | 553 | . | . | . | . | T | . | . | 1.74 | * | * | F | 2.86 | 1.89 |
| Cys | 554 | . | . | . | . | T | T | . | 2.44 | * | . | F | 3.40 | 2.15 |
| Lys | 555 | . | . | . | . | T | T | . | 2.53 | * | . | F | 3.06 | 1.90 |
| Asp | 556 | . | . | . | . | . | T | C | 2.57 | * | . | F | 2.52 | 1.70 |
| Pro | 557 | . | . | . | . | . | T | C | 2.46 | * | . | F | 2.52 | 5.49 |
| Glu | 558 | . | . | . | . | . | . | C | 2.11 | . | . | F | 2.32 | 4.41 |
| Pro | 559 | . | . | . | . | T | T | . | 2.43 | . | * | F | 2.72 | 2.62 |
| Gln | 560 | . | . | . | . | T | T | . | 2.50 | . | * | F | 2.76 | 1.67 |
| Asn | 561 | . | . | . | . | T | T | . | 2.26 | * | * | F | 3.40 | 1.89 |
| Gly | 562 | . | . | . | . | T | T | . | 1.80 | * | * | F | 2.76 | 1.92 |
| Gly | 563 | . | . | . | . | T | T | . | 0.99 | * | * | F | 2.27 | 0.59 |
| Arg | 564 | . | . | B | . | . | T | . | 0.86 | * | . | F | 0.93 | 0.30 |
| Tyr | 565 | . | . | B | . | . | T | . | 0.97 | . | . | . | 0.44 | 0.30 |
| Cys | 566 | . | . | B | . | . | T | . | 1.08 | . | . | . | 1.00 | 0.60 |
| Leu | 567 | . | . | B | . | . | . | . | 0.83 | . | * | . | 1.40 | 0.60 |
| Gly | 568 | . | . | B | . | . | . | . | 1.22 | . | * | F | 1.55 | 0.39 |
| Arg | 569 | . | . | B | . | . | . | . | 0.87 | . | * | F | 2.30 | 1.45 |
| Arg | 570 | . | . | . | . | T | . | . | 1.11 | * | * | F | 3.00 | 2.75 |
| Ala | 571 | . | . | . | . | T | . | . | 1.48 | * | * | F | 2.70 | 4.82 |
| Lys | 572 | . | . | . | . | T | . | . | 1.62 | * | * | F | 2.40 | 3.30 |
| Tyr | 573 | . | . | . | . | T | T | . | 1.93 | * | . | F | 1.85 | 0.90 |
| Gln | 574 | . | . | . | . | T | T | . | 1.51 | . | . | F | 1.10 | 1.22 |
| Ser | 575 | . | . | . | . | T | T | . | 1.40 | . | * | . | 0.50 | 0.88 |
| Cys | 576 | . | . | . | . | T | T | . | 1.99 | . | . | . | 0.50 | 0.97 |
| His | 577 | . | A | B | . | . | . | . | 1.28 | . | . | . | 0.60 | 0.97 |
| Thr | 578 | . | A | . | . | T | . | . | 1.31 | . | . | F | 0.85 | 0.39 |
| Glu | 579 | . | A | . | . | T | . | . | 1.10 | . | . | F | 1.00 | 1.12 |
| Glu | 580 | . | A | . | . | T | . | . | 1.40 | . | . | F | 1.64 | 1.27 |
| Cys | 581 | . | A | B | . | . | . | . | 1.72 | . | * | F | 1.58 | 1.47 |
| Pro | 582 | . | . | . | . | . | T | C | 1.80 | . | * | F | 2.37 | 0.84 |
| Pro | 583 | . | . | . | . | T | T | . | 1.81 | * | . | F | 2.91 | 0.97 |
| Asp | 584 | . | . | . | . | T | T | . | 1.11 | * | . | F | 3.40 | 2.43 |
| Gly | 585 | . | . | . | . | T | T | . | 1.22 | * | . | F | 3.06 | 1.36 |
| Lys | 586 | . | A | . | . | T | . | . | 1.89 | * | . | F | 2.32 | 1.72 |
| Ser | 587 | A | A | . | . | . | . | . | 2.10 | . | . | F | 1.58 | 1.79 |
| Phe | 588 | A | A | . | . | . | . | . | 2.31 | . | . | F | 1.24 | 3.13 |
| Arg | 589 | A | A | . | . | . | . | . | 1.64 | . | . | F | 0.90 | 2.71 |
| Glu | 590 | A | A | . | . | . | . | . | 1.99 | . | . | F | 0.60 | 1.08 |
| Gln | 591 | A | A | . | . | . | . | . | 1.99 | . | . | F | 0.90 | 2.17 |
| Gln | 592 | A | A | . | . | . | . | . | 2.04 | . | * | F | 0.90 | 2.21 |
| Cys | 593 | A | A | . | . | . | . | . | 2.74 | . | * | F | 1.15 | 2.00 |
| Glu | 594 | . | A | . | . | T | . | . | 2.04 | . | . | F | 1.50 | 1.86 |
| Lys | 595 | . | A | . | . | T | . | . | 1.80 | . | . | F | 1.75 | 1.08 |
| Tyr | 596 | . | . | . | . | T | . | . | 1.80 | . | . | . | 2.05 | 3.17 |
| Asn | 597 | . | . | . | . | T | T | . | 1.56 | . | . | . | 2.50 | 2.94 |
| Ala | 598 | . | . | . | . | T | T | . | 1.91 | . | . | . | 1.35 | 2.30 |
| Tyr | 599 | . | . | B | . | . | T | . | 1.91 | . | . | . | 0.70 | 2.12 |
| Asn | 600 | . | . | B | . | . | T | . | 1.27 | . | * | . | 0.75 | 2.20 |
| Tyr | 601 | . | . | B | . | . | . | . | 1.51 | . | . | . | 0.25 | 2.16 |
| Thr | 602 | . | . | B | . | . | . | . | 1.17 | . | * | F | 0.70 | 2.30 |
| Asp | 603 | . | . | B | . | . | T | . | 1.76 | . | * | F | 1.75 | 1.42 |
| Met | 604 | . | . | B | . | . | T | . | 1.19 | . | * | F | 2.00 | 1.45 |
| Asp | 605 | . | . | . | . | T | T | . | 0.38 | * | . | F | 2.50 | 0.83 |
| Gly | 606 | . | . | B | . | . | T | . | 0.62 | * | * | F | 1.85 | 0.41 |
| Asn | 607 | . | . | B | B | . | . | . | 0.64 | * | * | F | 0.60 | 0.72 |
| Leu | 608 | A | . | . | B | . | . | . | −0.21 | * | * | . | −0.10 | 0.45 |
| Leu | 609 | . | . | B | B | . | . | . | 0.18 | * | * | . | −0.35 | 0.34 |
| Gln | 610 | . | . | B | B | . | . | . | 0.22 | * | . | . | −0.60 | 0.33 |
| Trp | 611 | . | . | B | B | . | . | . | 0.32 | * | . | . | −0.60 | 0.79 |
| Val | 612 | . | . | B | B | . | . | . | −0.27 | * | . | . | −0.45 | 1.50 |
| Pro | 613 | . | . | B | . | . | T | . | 0.20 | * | . | . | −0.20 | 0.88 |
| Lys | 614 | . | . | B | . | . | T | . | 0.16 | * | * | . | −0.20 | 0.82 |
| Tyr | 615 | . | . | B | . | . | T | . | −0.14 | . | . | . | 0.10 | 0.82 |
| Ala | 616 | . | . | . | . | T | T | . | −0.07 | * | * | . | 0.50 | 0.71 |
| Gly | 617 | . | . | . | . | T | . | . | 0.90 | * | . | . | 0.64 | 0.55 |
| Val | 618 | . | . | B | . | . | . | . | 1.11 | . | * | . | 0.58 | 0.69 |
| Ser | 619 | . | . | B | . | . | T | . | 1.18 | . | * | F | 2.32 | 1.14 |
| Pro | 620 | . | . | B | . | . | T | . | 0.76 | . | * | F | 2.66 | 2.26 |
| Arg | 621 | . | . | . | . | T | T | . | 1.39 | . | * | F | 3.40 | 1.63 |
| Asp | 622 | . | . | . | . | T | T | . | 0.92 | . | * | F | 3.06 | 2.43 |
| Arg | 623 | . | A | . | . | T | . | . | 1.08 | . | * | F | 2.32 | 1.30 |
| Cys | 624 | . | A | B | . | . | . | . | 0.71 | * | * | F | 1.43 | 0.57 |
| Lys | 625 | . | A | B | . | . | . | . | 1.03 | * | * | . | 0.64 | 0.18 |
| Leu | 626 | . | A | B | . | . | . | . | 0.33 | * | * | . | 0.30 | 0.18 |
| Phe | 627 | . | A | B | . | . | . | . | 0.44 | . | * | . | 0.04 | 0.35 |
| Cys | 628 | . | A | B | . | . | . | . | −0.01 | . | * | . | 0.98 | 0.34 |
| Arg | 629 | . | A | B | . | . | . | . | 0.77 | * | * | . | 1.32 | 0.41 |
| Ala | 630 | A | . | . | . | . | T | . | 0.42 | * | * | . | 2.36 | 0.92 |
| Arg | 631 | . | . | . | . | T | T | . | 1.23 | . | * | F | 3.40 | 2.31 |
| Gly | 632 | . | . | . | . | T | T | . | 1.23 | . | * | F | 3.06 | 2.04 |
| Arg | 633 | . | . | . | . | T | T | . | 1.94 | . | * | F | 2.72 | 1.75 |
| Ser | 634 | A | A | . | . | . | . | . | 0.98 | * | * | F | 1.58 | 1.79 |
| Glu | 635 | A | A | . | . | . | . | . | 0.87 | * | * | F | 1.24 | 1.34 |
| Phe | 636 | A | A | . | . | . | . | . | 0.76 | * | * | F | 0.45 | 0.59 |
| Lys | 637 | A | A | . | . | . | . | . | 0.51 | * | * | . | 0.30 | 0.77 |
| Val | 638 | A | A | . | . | . | . | . | 0.44 | * | * | . | 0.30 | 0.45 |
| Phe | 639 | A | A | . | . | . | . | . | −0.11 | . | . | . | 0.45 | 1.03 |
| Glu | 640 | A | A | . | . | . | . | . | −1.00 | * | . | . | 0.30 | 0.38 |
| Ala | 641 | A | . | . | B | . | . | . | −0.30 | * | . | . | −0.30 | 0.36 |
| Lys | 642 | A | . | . | B | . | . | . | −0.69 | . | . | . | 0.30 | 0.70 |
| Val | 643 | A | . | . | B | . | . | . | −0.14 | . | . | . | 0.60 | 0.40 |
| Ile | 644 | A | . | . | B | . | . | . | −0.26 | . | * | F | 0.45 | 0.57 |
| Asp | 645 | . | . | B | B | . | . | . | −0.92 | . | . | F | 0.45 | 0.23 |
| Gly | 646 | . | . | B | B | . | . | . | −0.68 | * | . | F | −0.45 | 0.17 |
| Thr | 647 | . | . | B | B | . | . | . | −0.93 | * | . | F | −0.15 | 0.24 |
| Leu | 648 | . | . | . | B | . | . | C | −0.08 | . | . | F | 0.05 | 0.22 |
| Cys | 649 | . | . | . | B | T | . | . | 0.50 | * | * | . | 0.10 | 0.39 |
| Gly | 650 | . | . | . | . | . | T | C | −0.31 | . | . | F | 0.45 | 0.39 |
| Pro | 651 | . | . | . | . | T | T | . | −0.56 | . | . | F | 0.65 | 0.39 |
| Glu | 652 | A | . | . | . | . | T | . | −1.13 | . | . | F | 0.25 | 0.73 |
| Thr | 653 | A | . | . | . | . | T | . | −0.99 | . | . | F | 0.25 | 0.52 |
| Leu | 654 | A | . | . | B | . | . | . | −1.18 | * | * | . | −0.30 | 0.18 |
| Ala | 655 | . | . | B | B | . | . | . | −0.72 | * | * | . | −0.60 | 0.08 |
| Ile | 656 | . | . | B | B | . | . | . | −0.86 | . | * | . | −0.60 | 0.10 |
| Cys | 657 | . | . | B | B | . | . | . | −0.86 | . | * | . | −0.60 | 0.13 |
| Val | 658 | A | . | . | B | . | . | . | −1.21 | . | * | . | −0.30 | 0.21 |
| Arg | 659 | . | . | B | B | . | . | . | −1.26 | . | * | . | −0.30 | 0.16 |
| Gly | 660 | . | . | . | B | T | T | . | −0.62 | . | * | F | 0.25 | 0.23 |
| Gln | 661 | . | . | B | B | . | . | . | −0.32 | . | * | F | 0.45 | 0.61 |
| Cys | 662 | . | . | B | B | . | . | . | 0.00 | . | * | . | 0.30 | 0.32 |
| Val | 663 | . | . | B | B | . | . | . | 0.19 | . | * | . | 0.30 | 0.32 |
| Lys | 664 | . | . | B | . | . | T | . | 0.08 | . | * | . | 0.10 | 0.10 |
| Ala | 665 | . | . | B | . | . | T | . | 0.39 | * | . | . | 0.70 | 0.30 |
| Gly | 666 | . | . | B | . | . | T | . | −0.47 | * | . | . | 0.70 | 0.56 |
| Cys | 667 | . | . | B | . | . | T | . | −0.66 | * | * | . | 0.70 | 0.21 |
| Asp | 668 | . | . | B | B | . | . | . | 0.20 | * | * | . | −0.30 | 0.15 |
| His | 669 | . | . | B | B | . | . | . | −0.14 | * | . | . | 0.30 | 0.26 |
| Val | 670 | . | . | B | B | . | . | . | 0.23 | * | . | . | 0.30 | 0.64 |
| Val | 671 | . | . | B | B | . | . | . | 0.69 | * | . | . | 0.64 | 0.59 |
| Asp | 672 | . | . | B | B | . | . | . | 1.40 | * | . | F | 1.13 | 0.86 |
| Ser | 673 | . | . | B | . | . | T | . | 0.59 | * | . | F | 2.32 | 2.31 |
| Pro | 674 | A | . | . | . | . | T | . | 0.62 | * | . | F | 2.66 | 2.56 |
| Arg | 675 | . | . | . | . | T | T | . | 1.52 | * | . | F | 3.40 | 2.56 |
| Lys | 676 | . | . | . | . | T | T | . | 1.71 | * | . | F | 3.06 | 3.82 |
| Leu | 677 | . | . | . | . | T | . | . | 1.37 | * | . | F | 2.52 | 1.33 |
| Asp | 678 | . | . | . | . | T | T | . | 0.81 | * | . | F | 2.23 | 0.67 |
| Lys | 679 | . | . | B | . | . | T | . | 0.36 | * | . | F | 1.49 | 0.25 |
| Cys | 680 | . | . | B | . | . | T | . | −0.10 | * | . | . | 0.70 | 0.16 |
| Gly | 681 | . | . | B | . | . | T | . | −0.49 | * | . | . | 0.70 | 0.10 |
| Val | 682 | . | . | B | . | . | . | . | 0.37 | * | . | . | −0.10 | 0.05 |
| Cys | 683 | . | . | B | . | . | T | . | 0.02 | . | . | . | 0.10 | 0.18 |
| Gly | 684 | . | . | . | . | T | T | . | −0.02 | . | . | F | 1.59 | 0.18 |
| Gly | 685 | . | . | . | . | T | T | . | 0.34 | . | . | F | 1.93 | 0.38 |
| Lys | 686 | . | . | . | . | T | T | . | 0.02 | . | . | F | 2.27 | 0.96 |
| Gly | 687 | . | . | . | . | T | . | . | 0.99 | . | . | F | 2.41 | 0.52 |
| Asn | 688 | . | . | . | . | T | T | . | 1.70 | . | . | F | 3.40 | 1.03 |
| Ser | 689 | . | . | B | . | . | T | . | 1.19 | . | . | F | 2.66 | 1.03 |
| Cys | 690 | . | . | B | . | . | T | . | 1.23 | . | . | F | 2.34 | 0.77 |
| Arg | 691 | . | . | B | . | . | T | . | 0.84 | . | . | F | 2.17 | 0.64 |
| Lys | 692 | . | . | B | . | . | . | . | 0.89 | * | . | F | 1.80 | 0.47 |
| Val | 693 | . | . | B | . | . | T | . | 0.08 | * | . | F | 1.98 | 1.18 |
| Ser | 694 | . | . | B | . | . | T | . | 0.07 | * | . | F | 1.70 | 0.50 |
| Gly | 695 | . | . | B | . | . | T | . | 0.52 | * | . | F | 0.93 | 0.36 |
| Ser | 696 | . | . | B | . | . | T | . | 0.10 | * | . | F | 0.46 | 0.75 |
| Leu | 697 | . | . | B | . | . | . | . | 0.06 | . | * | F | 0.39 | 0.81 |
| Thr | 698 | . | . | B | . | . | . | . | 0.67 | . | . | F | 0.37 | 1.31 |
| Pro | 699 | . | . | B | . | . | T | . | 0.62 | . | . | F | 0.10 | 1.53 |
| Thr | 700 | . | . | . | . | T | T | . | 0.72 | . | . | F | 0.50 | 1.84 |
| Asn | 701 | . | . | B | . | . | T | . | 1.02 | . | . | F | 0.10 | 2.00 |
| Tyr | 702 | . | . | . | . | T | T | . | 1.83 | * | . | . | 0.35 | 2.08 |
| Gly | 703 | . | . | . | . | T | T | . | 1.26 | * | . | . | 0.65 | 2.41 |
| Tyr | 704 | . | . | . | . | T | T | . | 0.61 | * | . | . | 0.35 | 1.05 |
| Asn | 705 | . | . | B | . | . | T | . | 0.61 | * | . | . | −0.20 | 0.50 |
| Asp | 706 | . | . | B | . | . | T | . | −0.28 | * | . | . | 0.10 | 0.72 |
| Ile | 707 | . | . | B | B | . | . | . | −0.24 | * | . | . | −0.60 | 0.32 |
| Val | 708 | . | . | B | B | . | . | . | −0.49 | . | . | . | −0.30 | 0.31 |
| Thr | 709 | . | . | B | B | . | . | . | −0.59 | * | . | . | −0.60 | 0.19 |
| Ile | 710 | . | . | B | B | . | . | . | −1.18 | . | . | . | −0.60 | 0.27 |
| Pro | 711 | . | . | B | . | . | T | . | −1.49 | * | . | . | −0.20 | 0.36 |
| Ala | 712 | . | . | B | . | . | T | . | −0.60 | * | . | . | −0.20 | 0.36 |
| Gly | 713 | . | . | . | . | . | T | C | −0.63 | . | * | . | 0.00 | 0.83 |
| Ala | 714 | . | . | . | . | . | T | C | −0.32 | . | * | F | 0.15 | 0.38 |
| Thr | 715 | . | . | B | B | . | . | . | −0.29 | . | * | F | 0.45 | 0.62 |
| Asn | 716 | . | . | B | B | . | . | . | −0.03 | . | * | F | −0.15 | 0.47 |
| Ile | 717 | . | . | B | B | . | . | . | 0.56 | . | * | F | 0.45 | 0.92 |
| Asp | 718 | . | . | B | B | . | . | . | 1.01 | . | * | F | 0.60 | 1.11 |
| Val | 719 | . | . | B | B | . | . | . | 1.30 | . | * | F | 0.90 | 1.35 |
| Lys | 720 | . | . | B | B | . | . | . | 1.58 | . | * | F | 0.90 | 2.58 |
| Gln | 721 | . | . | B | . | . | . | . | 1.37 | . | * | F | 1.10 | 2.10 |
| Arg | 722 | . | . | B | . | . | . | . | 1.91 | . | * | F | 1.10 | 4.38 |
| Ser | 723 | . | . | . | . | . | . | C | 1.06 | * | * | F | 1.30 | 2.17 |
| His | 724 | . | . | . | . | . | T | C | 1.91 | * | * | F | 1.05 | 0.93 |
| Pro | 725 | . | . | . | . | . | T | C | 1.87 | . | * | F | 1.33 | 0.82 |
| Gly | 726 | . | . | . | . | T | T | . | 1.87 | * | * | F | 1.21 | 0.99 |
| Val | 727 | . | . | B | . | . | T | . | 1.41 | * | * | F | 1.84 | 1.21 |
| Gln | 728 | . | . | B | . | . | . | . | 1.71 | . | * | F | 1.77 | 0.77 |
| Asn | 729 | . | . | B | . | T | T | . | 1.50 | * | . | F | 2.80 | 1.26 |
| Asp | 730 | . | . | . | . | T | T | . | 0.90 | * | . | F | 1.92 | 2.66 |
| Gly | 731 | . | . | . | . | T | T | . | 0.66 | . | . | F | 1.64 | 1.27 |
| Asn | 732 | . | . | B | . | . | T | . | 0.70 | . | * | F | 0.81 | 0.80 |
| Tyr | 733 | . | A | B | . | . | . | . | 0.74 | . | . | . | −0.32 | 0.39 |
| Leu | 734 | . | A | B | . | . | . | . | 0.43 | * | . | . | −0.60 | 0.79 |
| Ala | 735 | . | A | B | . | . | . | . | −0.16 | * | . | . | −0.60 | 0.71 |
| Leu | 736 | . | A | B | . | . | . | . | 0.19 | . | . | . | −0.40 | 0.46 |
| Lys | 737 | . | A | B | . | . | . | . | −0.16 | . | . | F | 0.85 | 0.93 |
| Thr | 738 | . | . | B | . | . | T | . | 0.09 | . | . | F | 1.45 | 0.91 |
| Ala | 739 | A | . | . | . | . | T | . | 0.66 | . | . | F | 2.10 | 1.91 |
| Asp | 740 | . | . | B | . | . | T | . | 0.43 | . | . | F | 2.00 | 1.50 |
| Gly | 741 | . | . | B | . | . | T | . | 0.43 | . | * | F | 1.05 | 0.86 |
| Gln | 742 | . | . | B | . | . | . | . | 0.39 | . | * | F | 0.35 | 0.70 |
| Tyr | 743 | . | . | B | . | . | . | . | 0.36 | . | * | . | 0.30 | 0.67 |
| Leu | 744 | . | . | B | . | . | . | . | 0.94 | . | * | . | −0.20 | 0.67 |
| Leu | 745 | . | . | B | . | . | . | . | 0.13 | . | * | . | −0.40 | 0.63 |
| Asn | 746 | . | . | B | . | . | T | . | −0.11 | . | * | F | −0.05 | 0.33 |
| Gly | 747 | . | . | . | . | T | T | . | −1.00 | . | * | F | 0.35 | 0.40 |
| Asn | 748 | . | . | . | . | . | T | C | −1.06 | . | * | . | 0.00 | 0.34 |
| Leu | 749 | . | . | . | . | . | T | C | −0.83 | . | * | . | 0.00 | 0.29 |
| Ala | 750 | A | A | B | . | . | . | . | −0.91 | . | * | . | −0.60 | 0.29 |
| Ile | 751 | . | A | B | . | . | . | . | −0.91 | * | * | . | −0.60 | 0.13 |
| Ser | 752 | . | A | B | . | . | . | . | −0.57 | * | . | . | −0.60 | 0.27 |
| Ala | 753 | A | A | . | . | . | . | . | −0.57 | * | * | . | −0.30 | 0.46 |
| Ile | 754 | A | A | . | . | . | . | . | −0.64 | * | . | . | 0.45 | 1.09 |
| Glu | 755 | A | A | . | . | . | . | . | −0.87 | * | . | F | 0.45 | 0.57 |
| Gln | 756 | A | . | . | B | . | . | . | −0.83 | . | * | F | 0.45 | 0.47 |
| Asp | 757 | A | . | . | B | . | . | . | −0.49 | . | * | F | −0.15 | 0.49 |
| Ile | 758 | A | . | . | B | . | . | . | −0.24 | . | * | . | 0.60 | 0.57 |
| Leu | 759 | A | . | . | B | . | . | . | 0.33 | . | * | . | 0.30 | 0.33 |
| Val | 760 | A | . | . | B | . | . | . | −0.56 | . | * | . | 0.30 | 0.28 |
| Lys | 761 | A | . | . | B | . | . | . | −1.37 | . | * | F | −0.45 | 0.28 |
| Gly | 762 | . | . | B | B | . | . | . | −1.32 | . | * | F | −0.45 | 0.28 |
| Thr | 763 | . | . | B | B | . | . | . | −0.68 | . | * | F | 0.45 | 0.76 |
| Ile | 764 | . | . | B | B | . | . | . | −0.17 | . | . | F | −0.15 | 0.59 |
| Leu | 765 | . | . | B | B | . | . | . | 0.34 | . | * | . | −0.60 | 0.80 |
| Lys | 766 | . | . | B | B | . | . | . | 0.00 | . | * | F | −0.45 | 0.55 |
| Tyr | 767 | . | . | B | . | . | T | . | −0.54 | * | * | F | 0.40 | 1.05 |
| Ser | 768 | . | . | . | . | . | T | C | −0.82 | * | * | F | 0.45 | 0.89 |
| Gly | 769 | . | . | . | . | . | T | C | −0.24 | * | . | F | 0.45 | 0.45 |
| Ser | 770 | . | . | . | . | . | T | C | −0.24 | . | * | F | 0.15 | 0.42 |
| Ile | 771 | . | A | B | . | . | . | . | −0.29 | * | * | . | −0.60 | 0.26 |
| Ala | 772 | . | A | B | . | . | . | . | 0.07 | * | . | . | −0.30 | 0.45 |
| Thr | 773 | . | A | B | . | . | . | . | −0.44 | * | * | . | 0.30 | 0.66 |
| Leu | 774 | . | A | B | . | . | . | . | −0.10 | * | . | . | −0.30 | 0.77 |
| Glu | 775 | A | A | . | . | . | . | . | −0.10 | * | | . | 0.45 | 1.32 |
| Arg | 776 | . | A | B | . | . | . | . | 0.09 | . | . | F | 0.60 | 1.23 |
| Leu | 777 | . | A | . | . | T | . | . | 0.79 | . | . | F | 1.00 | 1.29 |
| Gln | 778 | . | A | . | . | T | . | . | 0.89 | . | . | F | 1.30 | 1.46 |
| Ser | 779 | . | A | . | . | T | . | . | 0.89 | . | . | F | 1.00 | 1.15 |
| Phe | 780 | . | . | B | . | . | . | . | 0.68 | * | . | F | 0.41 | 1.15 |
| Arg | 781 | . | . | . | . | . | . | C | 0.57 | . | * | F | 0.82 | 1.03 |
| Pro | 782 | . | . | . | . | . | . | C | 1.17 | * | . | F | 1.63 | 1.33 |
| Leu | 783 | . | . | . | . | . | T | C | 0.36 | * | . | F | 2.04 | 2.37 |
| Pro | 784 | . | . | . | . | . | T | C | 0.34 | * | * | F | 2.10 | 1.00 |
| Glu | 785 | . | . | . | . | . | T | C | 0.19 | * | * | F | 1.29 | 0.93 |
| Pro | 786 | . | . | B | . | . | T | . | 0.08 | * | * | F | 0.88 | 0.84 |
| Leu | 787 | . | . | B | B | . | . | . | −0.52 | . | * | F | 0.27 | 0.94 |
| Thr | 788 | . | . | B | B | . | . | . | −0.52 | . | * | . | −0.09 | 0.45 |
| Val | 789 | . | . | B | B | . | . | . | −0.62 | . | . | . | −0.60 | 0.24 |
| Gln | 790 | . | . | B | B | . | . | . | −1.48 | . | . | . | −0.60 | 0.42 |
| Leu | 791 | . | . | B | B | . | . | . | −1.48 | . | . | . | −0.60 | 0.21 |
| Leu | 792 | . | . | B | B | . | . | . | −1.01 | . | * | . | −0.60 | 0.45 |
| Thr | 793 | . | . | B | B | . | . | . | −0.70 | . | * | . | −0.60 | 0.26 |
| Val | 794 | . | . | B | . | . | T | . | −0.70 | * | . | F | 0.25 | 0.54 |
| Pro | 795 | . | . | B | . | . | T | . | −1.40 | * | . | F | 0.25 | 0.48 |
| Gly | 796 | . | . | B | . | . | T | . | −0.80 | * | . | F | −0.05 | 0.29 |
| Glu | 797 | . | . | B | . | . | T | . | −0.20 | . | * | F | 0.25 | 0.60 |
| Val | 798 | . | . | B | . | . | . | . | 0.16 | . | * | F | 0.05 | 0.60 |
| Phe | 799 | . | . | B | . | . | . | . | 0.16 | . | * | F | 1.00 | 1.22 |
| Pro | 800 | . | . | B | . | . | T | . | 0.41 | * | * | F | 1.25 | 0.52 |
| Pro | 801 | . | . | . | . | T | T | . | 0.51 | * | * | F | 2.00 | 1.41 |
| Lys | 802 | . | . | . | . | T | T | . | 0.20 | * | * | F | 1.60 | 2.55 |
| Val | 803 | . | . | B | . | . | T | . | 0.36 | . | * | F | 2.00 | 2.38 |
| Lys | 804 | . | . | B | B | . | . | . | 0.36 | . | * | F | 0.80 | 1.33 |
| Tyr | 805 | . | . | B | B | . | . | . | −0.29 | . | * | . | 0.00 | 0.58 |
| Thr | 806 | . | . | B | B | . | . | . | −0.29 | . | * | . | −0.20 | 0.58 |
| Phe | 807 | . | . | B | B | . | . | . | −0.33 | . | * | . | −0.40 | 0.45 |
| Phe | 808 | . | . | B | B | . | . | . | 0.52 | * | * | . | −0.60 | 0.46 |
| Val | 809 | . | . | B | . | . | T | . | −0.38 | * | * | . | −0.20 | 0.53 |
| Pro | 810 | . | . | B | . | . | T | . | −0.13 | . | * | F | −0.05 | 0.45 |
| Asn | 811 | . | . | . | . | T | T | . | −0.52 | . | * | F | 1.25 | 0.88 |
| Asp | 812 | . | . | . | . | T | T | . | −0.12 | . | * | F | 1.40 | 1.02 |
| Val | 813 | A | . | . | . | . | . | . | −0.02 | * | * | F | 0.65 | 0.89 |
| Asp | 814 | A | . | . | . | . | . | . | 0.83 | * | * | . | 0.50 | 0.54 |
| Phe | 815 | A | . | . | . | . | . | . | 0.74 | . | * | . | 0.80 | 0.57 |
| Ser | 816 | A | . | . | . | . | . | . | 0.44 | . | * | . | 0.65 | 1.02 |
| Met | 817 | A | . | . | . | . | . | . | 0.49 | . | * | . | 1.40 | 0.82 |
| Gln | 818 | A | . | . | . | . | T | . | 1.34 | . | * | F | 2.20 | 1.89 |
| Ser | 819 | . | . | . | . | . | T | C | 1.46 | . | * | F | 3.00 | 2.44 |
| Ser | 820 | . | . | . | . | . | T | C | 1.57 | . | * | F | 2.70 | 4.84 |
| Lys | 821 | A | . | . | . | . | T | . | 1.56 | . | * | F | 2.20 | 2.82 |
| Glu | 822 | A | . | . | . | . | . | . | 1.84 | . | * | F | 1.70 | 3.04 |
| Arg | 823 | A | . | . | B | . | . | . | 1.84 | * | * | F | 1.20 | 3.27 |
| Ala | 824 | A | . | . | B | . | . | . | 1.26 | * | * | F | 0.90 | 2.63 |
| Thr | 825 | . | . | B | B | . | . | . | 0.67 | * | * | F | 0.60 | 1.06 |
| Thr | 826 | . | . | B | B | . | . | . | 0.62 | * | * | F | −0.15 | 0.38 |
| Asn | 827 | . | . | B | B | . | . | . | 0.41 | * | * | . | −0.60 | 0.65 |
| Ile | 828 | . | . | B | B | . | . | . | −0.51 | * | * | . | −0.60 | 0.70 |
| Ile | 829 | . | . | B | B | . | . | . | −0.73 | * | . | . | −0.60 | 0.40 |
| Gln | 830 | . | A | B | . | . | . | . | −0.46 | * | . | . | −0.60 | 0.21 |
| Pro | 831 | . | A | B | . | . | . | . | −0.73 | * | . | . | −0.60 | 0.40 |
| Leu | 832 | . | A | B | . | . | . | . | −0.73 | * | * | . | −0.60 | 0.57 |
| Leu | 833 | . | A | B | . | . | . | . | −0.13 | . | . | . | −0.60 | 0.57 |
| His | 834 | . | A | B | . | . | . | . | −0.10 | . | * | . | −0.60 | 0.39 |
| Ala | 835 | . | A | B | B | . | . | . | −0.91 | . | * | . | −0.60 | 0.35 |
| Gln | 836 | . | A | B | B | . | . | . | −1.04 | . | . | . | −0.60 | 0.35 |
| Trp | 837 | . | A | B | B | . | . | . | −0.23 | . | . | . | −0.60 | 0.26 |
| Val | 838 | . | A | B | B | . | . | . | 0.29 | . | * | . | −0.60 | 0.42 |
| Leu | 839 | . | . | B | . | . | T | . | 0.02 | * | . | . | −0.20 | 0.26 |
| Gly | 840 | . | . | . | . | T | T | . | 0.61 | * | . | . | 0.45 | 0.33 |
| Asp | 841 | . | . | . | . | T | T | . | −0.06 | . | . | F | 1.15 | 0.76 |
| Trp | 842 | . | . | . | . | T | T | . | −0.07 | . | . | F | 2.00 | 0.50 |
| Ser | 843 | . | . | . | . | . | T | C | 0.49 | * | . | F | 2.05 | 0.67 |
| Glu | 844 | . | . | . | . | T | T | . | 0.99 | . | . | F | 2.50 | 0.54 |
| Cys | 845 | . | . | . | . | T | T | . | 0.67 | . | . | F | 1.65 | 0.74 |
| Ser | 846 | . | . | . | . | T | T | . | 0.32 | . | . | F | 2.00 | 0.30 |
| Ser | 847 | . | . | . | . | T | . | . | 0.02 | . | . | F | 1.55 | 0.17 |
| Thr | 848 | . | . | . | . | T | . | . | −0.02 | . | . | F | 0.70 | 0.32 |
| Cys | 849 | . | . | . | . | T | . | . | −0.31 | . | . | F | 0.45 | 0.24 |
| Gly | 850 | . | . | . | . | T | T | . | 0.36 | * | . | . | 0.20 | 0.18 |
| Ala | 851 | . | . | . | . | T | T | . | 0.77 | . | . | . | 0.20 | 0.22 |
| Gly | 852 | . | . | . | . | T | T | . | 1.18 | . | . | . | 0.50 | 0.81 |
| Trp | 853 | . | . | . | . | T | T | . | 1.18 | * | . | . | 1.25 | 1.60 |
| Gln | 854 | . | . | B | B | . | . | . | 0.99 | * | . | F | 0.60 | 2.29 |
| Arg | 855 | . | . | B | B | . | . | . | 1.33 | * | . | F | 0.60 | 1.72 |
| Arg | 856 | . | . | B | B | . | . | . | 1.26 | . | * | F | 0.90 | 2.83 |
| Thr | 857 | . | . | B | B | . | . | . | 1.71 | . | . | F | 1.05 | 0.87 |
| Val | 858 | . | . | B | B | . | . | . | 2.00 | . | . | . | 1.20 | 0.87 |
| Glu | 859 | . | . | B | B | . | . | . | 1.79 | . | . | . | 1.50 | 0.75 |
| Cys | 860 | . | . | . | . | T | . | . | 1.38 | . | * | . | 2.40 | 0.80 |
| Arg | 861 | . | . | . | . | T | . | . | 0.92 | . | . | F | 3.00 | 1.44 |
| Asp | 862 | . | . | . | . | . | T | C | 1.23 | . | * | F | 2.55 | 0.82 |
| Pro | 863 | . | . | . | . | T | T | . | 1.50 | . | * | F | 2.60 | 2.66 |
| Ser | 864 | . | . | . | . | T | T | . | 1.20 | . | * | F | 2.30 | 1.37 |
| Gly | 865 | . | . | . | . | T | T | . | 1.28 | . | . | F | 1.70 | 1.10 |
| Gln | 866 | A | . | . | . | . | . | . | 0.86 | . | * | F | 0.05 | 0.72 |
| Ala | 867 | . | . | B | . | . | . | . | 0.19 | . | * | F | 0.05 | 0.78 |
| Ser | 868 | . | . | B | . | . | . | . | 0.40 | . | * | . | −0.10 | 0.42 |
| Ala | 869 | A | . | . | . | . | . | . | 0.74 | . | * | . | −0.10 | 0.39 |
| Thr | 870 | A | . | . | . | . | T | . | 0.50 | * | . | . | 0.70 | 0.77 |
| Cys | 871 | A | . | . | . | . | T | . | −0.31 | * | . | . | 0.70 | 0.58 |
| Asn | 872 | A | . | . | . | . | T | . | 0.32 | * | . | . | 0.10 | 0.48 |
| Lys | 873 | A | . | . | . | . | T | . | 0.41 | . | . | F | 0.85 | 0.66 |
| Ala | 874 | A | . | . | . | . | . | . | 1.00 | * | . | F | 0.80 | 1.90 |
| Leu | 875 | A | . | . | . | . | . | . | 1.31 | * | . | F | 1.10 | 2.05 |
| Lys | 876 | A | . | . | . | . | T | . | 1.39 | . | . | F | 1.30 | 1.71 |
| Pro | 877 | A | . | . | . | . | T | . | 1.43 | . | . | F | 1.30 | 1.71 |
| Glu | 878 | A | . | . | . | . | T | . | 1.18 | . | . | F | 1.30 | 4.14 |
| Asp | 879 | A | . | . | . | . | T | . | 1.10 | . | . | F | 1.30 | 3.20 |
| Ala | 880 | A | . | . | . | . | . | . | 1.91 | . | . | F | 1.10 | 1.11 |
| Lys | 881 | A | . | . | . | . | T | . | 1.57 | . | . | F | 1.30 | 1.11 |
| Pro | 882 | A | . | . | . | . | T | . | 1.78 | * | . | F | 1.15 | 0.89 |
| Cys | 883 | A | . | . | . | . | T | . | 0.97 | * | . | F | 1.30 | 1.53 |
| Glu | 884 | A | . | . | . | . | T | . | 0.30 | . | . | F | 1.15 | 0.63 |
| Ser | 885 | A | A | . | . | . | . | . | 0.68 | * | . | F | −0.15 | 0.22 |
| Gln | 886 | . | A | B | . | . | . | . | −0.18 | * | . | F | −0.15 | 0.63 |
| Leu | 887 | . | A | B | . | . | . | . | −0.36 | . | . | . | −0.30 | 0.30 |
| Cys | 888 | . | A | B | . | . | . | . | −0.08 | . | . | . | −0.60 | 0.29 |
| Pro | 889 | . | A | B | . | . | . | . | −0.47 | . | . | . | −0.60 | 0.21 |
| Leu | 890 | . | . | B | . | . | . | . | −0.56 | . | . | . | −0.40 | 0.33 |

### DETAILED DESCRIPTION OF THE PREFERRED EMBODIMENTS

By screening cDNA libraries with cDNA encoding the anti-angiogenic domain of TSP-1, the present inventors have identified two novel proteins, METH1 and METH2 (also called VEGA-1 and VEGA-2, respectively, for vascular endothelial growth antagonist) which contain the anti-angiogenic domain of TSP-1, a metalloproteinase domain, and a disintegrin-like domain. The present inventors have demonstrated that both METH1 and METH2 have anti-angiogenic activity. METH1 is also called ITGL-TSP.

Thus, the present invention provides isolated nucleic acid molecules comprising a polynucleotide encoding a METH1 polypeptide having the amino acid sequence shown in SEQ ID NO:2, which was determined by sequencing a cloned cDNA. The METH1 protein of the present invention shares sequence homology with thrombospondin-1 and pNPI. The nucleotide sequence shown in SEQ ID NO:1 was obtained by sequencing a cDNA clone, which was deposited on Jan. 15, 1998 at the American Type Culture Collection, 10801 University Boulevard, Manassas, Va. 20110-2209, and given accession number 209581. The cDNA clone contained in ATCC Deposit No. 209581 contains a METH1 sequence, encoding amino acids 1 to 950 of SEQ ID NO:2.

The present invention also provides isolated nucleic acid molecules comprising a polynucleotide encoding a METH2 polypeptide having the amino acid sequence shown in SEQ ID NO:4, which was partially determined by sequencing a cloned cDNA. The METH2 protein of the present invention shares sequence homology with thrombospondin-1 and pNPI. The nucleotide sequence shown in SEQ ID NO:3 was partially obtained by sequencing a cDNA clone, which was deposited on Jan. 15, 1998 at the American Type Culture Collection, 10801 University Boulevard, Manassas, Va. 20110-2209, and given accession number 209582. The cDNA clone contained in ATCC Deposit No. 209582 contains a partial METH2 sequence, encoding amino acids 112-890 of SEQ ID NO:4. A cDNA clone containing the entire METH2 sequence was deposited on Mar. 14, 2000 at the American Type Culture Collection, 10801 University Boulevard, Manassas, Va. 20110-2209, and given accession number PTA 1478.

Nucleic Acid Molecules

Some of the nucleotide sequences determined by sequencing a DNA molecule herein were determined using an automated DNA sequencer (such as the Model 373 from Applied Biosystems, Inc.), and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

Using the information provided herein, such as the nucleotide sequence in SEQ ID NO: 1 or SEQ ID NO:3, a nucleic acid molecule of the present invention encoding a METH1 or METH2 polypeptide may be obtained using standard cloning and screening procedures, such as those for cloning cDNAs using mRNA as starting material. Illustrative of the invention, the nucleic acid molecule described in SEQ ID NO: 1 was discovered in a cDNA library derived from human heart and the nucleic acid molecule described in SEQ ID NO:3 was discovered in a cDNA library derived from human lung. The determined nucleotide sequence of the METH1 cDNA of SEQ ID NO: 1 contains an open reading frame encoding a protein of about 950 amino acid residues, including a predicted leader sequence of about 28 amino acid residues. The present inventors have determined that the nucleotide sequence of the METH2 cDNA of SEQ ID NO:3 contains an open reading frame encoding a protein of about 890 amino acid residues, including a predicted leader sequence of about 23 amino acid residues.

The present invention also provides the mature form(s) of the METH1 and METH2 proteins of the present invention. According to the signal hypothesis, proteins secreted by mammalian cells have a signal or secretory leader sequence which is cleaved from the mature protein once export of the growing protein chain across the rough endoplasmic reticulum has been initiated. Most mammalian cells and even insect cells cleave secreted proteins with the same specificity. However, in some cases, cleavage of a secreted protein is not entirely uniform, which results in two or more mature species on the protein. Further, it has long been known that the cleavage specificity of a secreted protein is ultimately determined by the primary structure of the complete protein, that is, it is inherent in the amino acid sequence of the polypeptide. Therefore, the present invention provides a nucleotide sequence encoding the mature METH1 polypeptide having the amino acid sequence encoded by the cDNA clone contained in the host identified as ATCC Deposit No. 209581 and as shown in SEQ ID NO:2. The present invention also provides a nucleotide sequence encoding the mature METH2 polypeptide having the amino acid sequence as shown in SEQ ID NO:4. By the mature METH1 protein having the amino acid sequence encoded by the cDNA clone contained in the host identified as ATCC Deposit No. 209581 is meant the mature form(s) of the METH1 protein produced by expression in a mammalian cell (e.g., COS cells, as described below) of the complete open reading frame encoded by the human DNA sequence of the clone contained in the vector in the deposited host. As indicated below, the mature METH1 having the amino acid sequence encoded by the cDNA clone contained in ATCC Deposit No. 209581 may or may not differ from the predicted “mature” METH1 protein shown in SEQ ID NO:2 (amino acids from about 29 to about 950) depending on the accuracy of the predicted cleavage site based on computer analysis; and the mature METH2 may or may not differ from the predicted “mature” METH2 protein shown in SEQ ID NO: 4 (amino acids from about 24 to about 890) depending on the accuracy of the predicted cleavage site based on computer analysis. Additionally, the mature form of the protein may then undergo even more processing after the prodomain has been cleaved (e.g., a second cleavage distal to the prodomain, located in the metalloprotease domain/cysteine-rich region). Thus, “mature” forms of the proteins encompass not only those forms produced by cleavage of the prodomain, but also other processed forms of the protein.

Methods for predicting whether a protein has a secretory leader as well as the cleavage point for that leader sequence are available. For instance, the methods of McGeoch (Virus Res. 3:271-286 (1985)) and von Heinje (Nucleic Acids Res. 14:4683-4690 (1986)) can be used. The accuracy of predicting the cleavage points of known mammalian secretory proteins for each of these methods is in the range of 75-80%. von Heinje, supra. However, the two methods do not always produce the same predicted cleavage point(s) for a given protein.

In the present case, the predicted amino acid sequence of the complete METH1 and METH2 polypeptides of the present invention were analyzed by a computer program (“PSORT”) (K. Nakai and M. Kanehisa, Genomics 14:897-911 (1992)), which is an expert system for predicting the cellular location of a protein based on the amino acid sequence. As part of this computational prediction of localization, the methods of McGeoch and von Heinje are incorporated. The analysis by the PSORT program predicted the cleavage site between amino acids 28 and 29 in SEQ ID NO:2 and amino acids 23 and 24 in SEQ ID NO:4. Thereafter, the complete amino acid sequences were further analyzed by visual inspection, applying a simple form of the (-1,-3) rule of von Heinje. von Heinje, supra. Thus, the leader sequence for the METH1 protein is predicted to consist of amino acid residues from about 1 to about 28 in SEQ ID NO:2, while the mature METH1 protein is predicted to consist of residues from about 29 to about 950; and the leader sequence for the METH2 protein is predicted to consist of amino acid residues from about 1 to about 23 in SEQ ID NO:4, while the mature METH2 protein is predicted to consist of residues from about 24 to about 890. An alternative predicted mature METH1 protein consists of residues 30 to 950 in SEQ ID NO:2. Another alternative predicted mature METH1 protein consists of residues 35 to 950 of SEQ ID NO:2. An alternative predicted mature METH2 protein consists of residues 31 to 890 of SEQ ID NO:4.

As one of ordinary skill would appreciate, due to the possibilities of sequencing errors, as well as the variability of cleavage sites for leaders in different known proteins, the predicted METH1 polypeptide encoded by the deposited cDNA comprises about 950 amino acids, but may be anywhere in the range of 910-990 amino acids; and the predicted leader sequence of this protein is about 28 amino acids, but may be anywhere in the range of about 18 to about 38 amino acids. An alternative predicted METH1 polypeptide is shown in SEQ ID NO: 126, encoded by SEQ ID NO: 125, and comprises an additional 18 amino acid residues on the N-terminus. Also, the predicted METH2 polypeptide comprises about 890 amino acids, but may be anywhere in the range of 850 to about 930 amino acids; and the predicted leader sequence of this protein is about 23 amino acids, but may be anywhere in the range of about 13 to about 33 amino acids.

As indicated, nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated” nucleic acid molecule(s) is intended a nucleic acid molecule, DNA or RNA, which has been removed from its native environment. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated nucleic acid molecules of the present invention include DNA molecules comprising an open reading frame (ORF) shown in SEQ ID NO:1; DNA molecules comprising the coding sequence for the mature METH1 protein; and DNA molecules which comprise a sequence substantially different from those described above but which, due to the degeneracy of the genetic code, still encode the METH1 protein. Also included are DNA molecules comprising an open reading frame (ORF) shown in SEQ ID NO:3; DNA molecules comprising the coding sequence for the mature METH2 protein; and DNA molecules which comprise a sequence substantially different from those described above but which, due to the degeneracy of the genetic code, still encode the METH2 protein. Of course, the genetic code is well known in the art. Thus, it would be routine for one skilled in the art to generate such degenerate variants.

Polynucleotides of the present invention encompass not only polynucleotides encoding the full length sequence, but polynucleotides encoding the mature, proprotein, processed forms of the protein, deletion mutants, substitution variants, allelic variants, analogs, derivatives, etc.

In another aspect, the invention provides isolated nucleic acid molecules encoding the METH1 or METH2 polypeptides having an amino acid sequence as encoded by the cDNA clones contained in the plasmids deposited as ATCC Deposit No. 209581 on Jan. 15, 1998 or ATCC Deposit No. 209582 on Jan. 15, 1998, respectively; or METH2 polypeptides having the amino acid sequence as encoded by the cDNA clone contained in the plasmid deposited as ATTC Deposit No. PTA 1478 on Mar. 14, 2000. In a further embodiment, nucleic acid molecules are provided encoding the mature METH1 or METH2 polypeptide or the full-length METH1 or METH2 polypeptide lacking the N-terminal methionine. The invention also provides an isolated nucleic acid molecule having the nucleotide sequence shown in SEQ ID NO: 1 or SEQ ID NO:3 or the nucleotide sequence of the METH1 or METH2 cDNA contained in the above-described deposited clones, or a nucleic acid molecule having a sequence complementary to one of the above sequences. Such isolated molecules, particularly DNA molecules, are useful as probes for gene mapping, by in situ hybridization with chromosomes, and for detecting expression of the METH1 or METH2 gene in human tissue, for instance, by Northern blot analysis.

The present invention is further directed to fragments of the isolated nucleic acid molecules described herein. By a fragment of an isolated nucleic acid molecule having the nucleotide sequence of the deposited cDNA or the nucleotide sequence shown in SEQ ID NO: 1 or SEQ ID NO:3 is intended fragments at least about 15 nt, and more preferably at least about 20 nt, still more preferably at least about 30 nt, and even more preferably, at least about 40 nt in length which are useful as diagnostic probes and primers as discussed herein. Of course, larger fragments 50, 100, 150, 200, 250, 300, 350, 400, 450, 500, 550, 600, 650, 700, 750, 800, 850, 900, 950, 1000, 1050, 1100, 1200, 1300, 1400, 1500, 1600, 1700, 1800, 1900, 2000, 2100, 2200, 2300, 2400, 2500, 2600, 2700, 2800, 2900, or 3000 nt in length are also useful according to the present invention as are fragments corresponding to most, if not all, of the nucleotide sequence of the deposited cDNA or as shown in SEQ ID NO:1 or SEQ ID NO:3. By a fragment at least 20 nt in length, for example, is intended fragments which include 20 or more contiguous bases from the nucleotide sequence of the deposited cDNA or the nucleotide sequence as shown in SEQ ID NO:1 or SEQ ID NO:3.

Preferred nucleic acid fragments of the present invention include nucleic acid molecules encoding epitope-bearing portions of the METH1 or METH2 protein. Methods for determining epitope-bearing portions of the METH1 and METH2 proteins are described in detail below.

Other preferred nucleic acid fragments of the present invention include nucleic acid molecules encoding: the metalloprotease domain of METH1, amino acids 235 to 459 in SEQ ID NO:2; the disintegrin domain of METH1, amino acids 460 to 544 in SEQ ID NO:2; the first TSP-like domain of METH1, amino acids 545 to 598 in SEQ ID NO:2; the second TSP-like domain of METH1, amino acids 841 to 894 in SEQ ID NO:2; the third TSP-like domain of METH1, amino acids 895 to 934 in SEQ ID NO:2; amino acids 536 to 613 in SEQ ID NO:2; amino acids 549 to 563 in SEQ ID NO:2; the metalloprotease domain of METH2, amino acids 214 to 439 in SEQ ID NO:4; the disintegrin domain of METH2, amino acids 440 to 529 in SEQ ID NO:4; the first TSP-like domain of METH2, amino acids 530 to 583 in SEQ ID NO:4; the second TSP-like domain of METH2, amino acids 837 to 890 in SEQ ID NO:4; amino acids 280 to 606 in SEQ ID NO:4; and amino acids 529 to 548 in SEQ ID NO:4; and nucleic acid molecules encoding combinations of these domains.

Thus, preferred embodiments include a nucleic acid molecule encoding a METH1 or METH2 protein lacking the signal sequence (cleavage occurs for METH1 somewhere about 1-24 to about 1-34 and about 1-23 to about 1-30 for METH2); a METH1 or METH2 protein lacking the signal sequence and the prodomain (cleavage for the prodomain can occur in METH1 between amino acids about 232 to 236 and in METH2 between amino acids about 211 to 215); a METH1 or METH2 protein lacking the signal sequence, the prodomain, and the metalloprotease domain; a METH1 or METH2 protein lacking the signal sequence, the prodomain, the metalloprotease domain, and the cysteine rich domain; a METH1 or METH2 protein lacking the signal sequence, the prodomain, the metalloprotease domain, cysteine rich domain and TSP1; a METH1 or METH2 protein lacking the signal sequence, the prodomain, the metalloprotease domain, cysteine rich domain, TSP1 and TSP2. Also preferred are polypeptides encoded by such nucleic acids.

Similarly, preferred embodiments include a nucleic acid encoding a METH1 protein lacking TSP3; a METH1 protein lacking TSP2 and TSP3; a METH1 protein lacking TSP3, TSP2, and TSP1; a METH1 protein lacking the cysteine-rich domain, TSP1, TSP2, and TSP3; a METH1 protein lacking the metalloprotease domain, the cysteine-rich domain, TSP1, TSP2 and TSP3; and a METH1 protein lacking the prodomain, the metalloprotease domain, the cysteine-rich domain, TSP1, TSP2, and TSP3; a METH2 protein lacking TSP2; a METH2 protein lacking TSP1 and TSP2; a METH2 protein lacking the cysteine-rich domain, TSP1 and TSP2; a METH2 protein lacking the metalloprotease domain, the cysteine-rich domain, TSP1 and TSP2; and a METH2 protein lacking the prodomain, the metalloprotease domain, the cysteine-rich domain, TSP1 and TSP2. Also preferred are polypeptide encoded by such nucleic acids.

Also preferred are nucleic acids encoding any combination of METH1 domains. For example, nucleic acid molecule encoding polypeptides comprising the following domains of METH1 are preferred: signal sequence and prodomain; signal sequence, prodomain and metalloprotease domain; signal sequence and metalloprotease domain; signal sequence, prodomain, metalloprotease domain, and cysteine rich domain; signal sequence and cysteine rich domain; signal sequence, metalloprotease domain and cysteine rich domain; signal sequence, prodomain, and cysteine rich domain; signal sequence, prodomain, metalloprotease domain, cysteine rich domain, and TSP1; signal sequence and TSP1; signal sequence, prodomain and TSP1; signal sequence, prodomain, metalloprotease domain and TSP1; signal sequence, metalloprotease domain, and TSP1; signal sequence, prodomain, cysteine rich domain and TSP1; signal sequence, cysteine rich domain and TSP1; signal sequence, metalloprotease domain, cysteine rich domain and TSP1; signal sequence, prodomain, metalloprotease domain, cysteine rich domain, TSP1 and TSP2; signal sequence and TSP2; signal sequence, prodomain and TSP2; signal sequence, metalloprotease domain and TSP2; signal sequence, cysteine rich domain and TSP2; signal sequence, TSP1 and TSP2; signal sequence, prodomain, metalloprotease domain and TSP2; signal sequence, prodomain, cysteine rich domain and TSP2; signal sequence, prodomain, TSP1 and TSP2; signal sequence, metalloprotease domain, cysteine rich domain and TSP2; signal sequence, metalloprotease domain, TSP 1 and TSP2; signal sequence, metalloprotease domain, cysteine rich domain, TSP1 and TSP2; signal sequence, prodomain, cysteine rich domain, TSP1 and TSP2; signal sequence and TSP3; signal sequence, prodomain and TSP3; signal sequence, prodomain, metalloprotease domain and TSP3; signal sequence, metalloprotease domain and TSP3; signal sequence, prodomain, metalloprotease domain, cysteine rich domain and TSP3; signal sequence, cysteine rich domain and TSP3; signal sequence, prodomain, cysteine rich domain and TSP3; signal sequence, prodomain, metalloprotease domain, cysteine rich domain, TSP1 and TSP3; signal sequence, TSP1 and TSP3; signal sequence, prodomain, TSP1 and TSP3; signal sequence, prodomain, metalloprotease domain, TSP1 and TSP3; signal sequence, prodomain, cysteine rich domain, TSP1 and TSP3; signal sequence, TSP2 and TSP3; signal sequence, prodomain, cysteine rich domain, TSP1, TSP2 and TSP3; signal sequence, prodomain, metalloprotease domain, TSP 1, TSP2 and TSP3; signal sequence, metalloprotease domain, TSP1, TSP2 and TSP3; signal sequence, TSP1, TSP2 and TSP3; signal sequence, metalloprotease domain, cysteine rich domain, TSP1, TSP2 and TSP3; signal sequence, prodomain, metalloprotease domain, TSP1 and TSP2; signal sequence, prodomain, metalloprotease domain, cysteine rich domain, and TSP2; signal sequence, prodomain, metalloprotease domain, cysteine rich domain, TSP2 and TSP3; signal sequence, TSP1, TSP2 and TSP3; signal sequence, cysteine rich domain, TSP1 and TSP2; signal sequence, cysteine rich domain, TSP1 and TSP3; signal sequence, cysteine rich domain, TSP2 and TSP3; signal sequence, cysteine rich domain, TSP1, TSP2, and TSP3; signal sequence, metalloprotease domain , cysteine rich domain, and TSP3; sign al sequence, metalloprotease domain, cysteine rich domain, TSP1 and TSP3; signal sequence, metalloprotease domain, cysteine rich domain, TSP2 and TSP3; signal sequence, metalloprotease domain, TSP1 and TSP3; signal sequence, metalloprotease domain, TSP2 and TSP3; signal sequence, prodomain, metalloprotease domain, TSP1 and TSP3; signal sequence, prodomain, metalloprotease domain, TSP2 and TSP3; prodomain and metalloprotease domain; prodomain and cysteine rich domain; prodomain and TSP1; prodomain and TSP2; prodomain and TSP3; prodomain, metalloprotease domain and cysteine rich domain; prodomain, metalloprotease domain and TSP1; prodomain, metalloprotease domain and TSP2; prodomain, metalloprotease domain and TSP3; prodomain, metalloprotease domain, cysteine rich domain and TSP1; prodomain, metalloprotease domain, cysteine rich domain and TSP2; prodomain, metalloprotease domain, cysteine rich domain and TSP3; prodomain, cysteine rich domain and TSP 1; prodomain, cysteine rich domain and TSP2; prodomain, cysteine rich domain and TSP3; prodomain, metalloprotease domain, cysteine rich domain, TSP 1 and TSP2; prodomain, metalloprotease domain, cysteine rich domain, TSP1, TSP2 and TSP3; prodomain, cysteine rich domain, TSP1 and TSP2; prodomain, metalloprotease domain, TSP1 and TSP2; prodomain, metalloprotease domain, cysteine rich domain, TSP2 and TSP3; prodomain, cysteine rich domain, TSP1 and TSP3; prodomain, cysteine rich domain, TSP2 and TSP3; prodomain, TSP1 and TSP2; prodomain, TSP1 and TSP3; prodomain, TSP2 and TSP3; prodomain, metalloprotease domain, TSP1 and TSP2; prodomain, metalloprotease domain, TSP1 and TSP3; prodomain, metalloprotease domain, TSP2 and TSP3; prodomain, metalloprotease domain, cysteine rich domain, TSP2 and TSP3; prodomain, TSP1 and TSP2; prodomain, TSP1 and TSP3; prodomain, TSP2 and TSP3; prodomain, metalloprotease domain, TSP1 and TSP2; prodomain, metalloprotease domain, TSP1 and TSP3; prodomain, metalloprotease domain, TSP2 and TSP3; prodomain, metalloprotease domain, cysteine domain, TSP1 and TSP3; prodomain, cysteine rich domain, TSP1, TSP2 and TSP3; prodomain, metalloprotease domain, TSP1, TSP2, and TSP3; metalloprotease domain and cysteine rich domain; metalloprotease domain and TSP1; metalloprotease domain and TSP2; metalloprotease domain and TSP3; metalloprotease domain, cysteine rich domain and TSP1; metalloprotease domain, cysteine rich domain and TSP2; metalloprotease domain, cysteine rich domain and TSP3; metalloprotease domain, cysteine rich domain, TSP1 and TSP2; metalloprotease domain, cysteine rich domain, TSP1, TSP2 and TSP3; metalloprotease domain, cysteine rich domain, TSP1 and TSP3; metalloprotease domain, cysteine rich domain, TSP2 and TSP3; metalloprotease domain, TSP1 and TSP2; metalloprotease domain, TSP1 and TSP3; metalloprotease domain, TSP2 and TSP3; metalloprotease domain, TSP1, TSP2 and TSP3; cysteine rich domain and TSP1; cysteine rich domain and TSP2; cysteine rich domain and TSP3; cysteine rich domain, TSP1 and TSP2; cysteine rich domain, TSP1 and TSP3; cysteine rich domain, TSP2 and TSP3; cysteine rich domain, TSP1, TSP2 and TSP3; TSP1 and TSP2; TSP1 and TSP3; TSP2 and TSP3; and/or TSP1, TSP2 and TSP3. These domains may be present in the METH1 molecule in the same order or a different order than in the naturally occurring molecule. Also preferred are polypeptides encoded by such nucleic acids.

Also preferred are nucleic acids encoding any combination of METH2 domains. For example, nucleic acid molecule encoding polypeptides comprising the following domains of METH2 are preferred: signal sequence and prodomain; signal sequence, prodomain and metalloprotease domain; signal sequence and metalloprotease domain; signal sequence, prodomain, metalloprotease domain, and cysteine rich domain; signal sequence and cysteine rich domain; signal sequence, metalloprotease domain and cysteine rich domain; signal sequence, prodomain, and cysteine rich domain; signal sequence, prodomain, metalloprotease domain, cysteine rich domain, and TSP1; signal sequence and TSP1; signal sequence, prodomain and TSP1; signal sequence, prodomain, metalloprotease domain and TSP 1; signal sequence, metalloprotease domain, and TSP 1; signal sequence, prodomain, cysteine rich domain and TSP1; signal sequence, cysteine rich domain and TSP1; signal sequence, metalloprotease domain, cysteine rich domain and TSP1; signal sequence, prodomain, metalloprotease domain, cysteine rich domain, TSP1 and TSP2; signal sequence and TSP2; signal sequence, prodomain and TSP2; signal sequence, metalloprotease domain and TSP2; signal sequence, cysteine rich domain and TSP2; signal sequence, TSP1 and TSP2; signal sequence, prodomain, metalloprotease domain and TSP2; signal sequence, prodomain, cysteine rich domain and TSP2; signal sequence, prodomain, TSP1 and TSP2; signal sequence, metalloprotease domain, cysteine rich domain and TSP2; signal sequence, metalloprotease domain, TSP1 and TSP2; signal sequence, metalloprotease domain, cysteine rich domain, TSP1 and TSP2; signal sequence, prodomain, cysteine rich domain, TSP1 and TSP2; signal sequence, prodomain, metalloprotease domain, TSP1 and TSP2; signal sequence, prodomain, metalloprotease domain, cysteine rich domain, and TSP2; signal sequence, cysteine rich domain, TSP1 and TSP2; prodomain and metalloprotease domain; prodomain and cysteine rich domain; prodomain and TSP1; prodomain and TSP2; prodomain, metalloprotease domain and cysteine rich domain; prodomain, metalloprotease domain and TSP1; prodomain, metalloprotease domain and TSP2; prodomain, metalloprotease domain, cysteine rich domain and TSP1; prodomain, metalloprotease domain, cysteine rich domain and TSP2; prodomain, cysteine rich domain and TSP1; prodomain, cysteine rich domain and TSP2; prodomain, metalloprotease domain, cysteine rich domain, TSP 1 and TSP2; prodomain, cysteine rich domain, TSP1 and TSP2; prodomain, metalloprotease domain, TSP1 and TSP2; prodomain, metalloprotease domain, TSP1 and TSP2; prodomain, TSP1 and TSP2; prodomain, metalloprotease domain, TSP1 and TSP2; metalloprotease domain and cysteine rich domain; metalloprotease domain and TSP1; metalloprotease domain and TSP2; metalloprotease domain, cysteine rich domain and TSP1; metalloprotease domain, cysteine rich domain and TSP2; metalloprotease domain, cysteine rich domain, TSP 1 and TSP2; metalloprotease domain, TSP1 and TSP2; cysteine rich domain and TSP1; cysteine rich domain and TSP2; cysteine rich domain, TSP1 and TSP2. These domains may be present in the METH2 molecule in the same order or a different order than in the naturally occurring molecule. Also preferred are polypeptides encoded by such nucleic acids.

Additionally, METH1 and METH2 domains may be combined to form hybrid molecules. Any domain of METH1 may be combined with any domain of METH2 to form a hybrid molecule. For example, the TSP1 domain of METH1 may be replaced with the TSP1 domain of METH2 to form a hybrid molecule, leaving the remainder of the METH1 molecule intact. Also, the TSP1 domain of METH1 may be replaced with the TSP2 domain of METH2 to form a hybrid molecule, leaving the remainder of the METH1 molecule intact. Additionally, the TSP1 domain of METH1 may be combined with the TSP2 domain of METH2 to form a hybrid molecule, without any additional METH1 and/or METH2 sequences. These domains may be present in the same or a different order as occurs in the naturally occurring molecules. Also preferred are polypeptides encoded by such nucleic acids.

Further embodiments include nucleic acids encoding a METH1 or METH2 polypeptide in which: one or more TSP domains have been replaced with other known TSP domains; the metalloprotease domain has been replaced with another known metalloprotease domain; the disintegrin domain has been replaced with another known disintegrin domain. One or more domains may be replaced in this manner. For example, the both the metalloprotease and disintegrin domains may be replaced. Alternatively, all three TSP domains may be replaced. Also preferred are polypeptides encoded by such nucleic acids.

Preferred embodiments are polynucleotides encoding the amino acid sequence of SEQ ID NO:2 or SEQ ID NO:4 except for several, 5-10, 1-5,1-3, 1-2 or 1 amino acid residues are substituted, deleted or added, in any combination.

In addition, the present inventors have identified the following cDNA clones related to portions of the sequence shown in SEQ ID NO:1: HOUCQ17RA (SEQ ID NO: 14), HPLBM11R (SEQ ID NO: 15), HGBI07R (SEQ ID NO: 16), HNTMA49R (SEQ ID NO:17), HNALE27R (SEQ ID NO:18), and HIBDB45R (SEQ ID NO:19).

The following public ESTs, which relate to portions of SEQ ID NO: 1, have also been identified: D67076 (SEQ ID NO:20), AB001735 (SEQ ID NO:21), X14787 (SEQ ID NO:22), U64857 (SEQ ID NO:23), X04665 (SEQ ID NO:24), M64866 (SEQ ID NO:25), L07803 (SEQ ID NO:26), U08006 (SEQ ID NO:27), M16974 (SEQ ID NO:28), L13855 (SEQ ID NO:29), AL021529 (SEQ ID NO:30), D86074 (SEQ ID NO:31), L05390 (SEQ ID NO:32), Z69361 (SEQ ID NO:33), X99599 (SEQ ID NO:34), AF018073 (SEQ ID NO:35), L23760 (SEQ ID NO:36), Z46970 (SEQ ID NO:37), AC004449 (SEQ ID NO:38), Z69589 (SEQ ID NO:39), Z22279 (SEQ ID NO:40), X17524 (SEQ ID NO:41), AI126019 (SEQ ID NO:103), AI571069 (SEQ ID NO:104), AI148739 (SEQ ID NO:105), AI335849 (SEQ ID NO:106), AA677116 (SEQ ID NO:107), H27128 (SEQ ID NO:108), AA368429 (SEQ ID NO:109), AA345812 (SEQ ID NO:110), AA373718 (SEQ ID NO:111), AI537518 (SEQ ID NO:112), N88341 (SEQ ID NO: 113), C₀₃₆₀₀ (SEQ ID NO: 114), AA066142 (SEQ ID NO: 115), AI40095 (SEQ ID NO:94), AA288689 (SEQ ID NO:116), AI464076 (SEQ ID NO:97), R13547 (SEQ ID NO:117), R19976 (SEQ ID NO:118), Z43925 (SEQ ID NO:119), AA670987 (SEQ ID NO:120), AA635657 (SEQ ID NO:96), W24878 (SEQ ID NO:121), W47316 (SEQ ID NO: 122), W35345 (SEQ ID NO: 123), and N27243 (SEQ ID NO: 124).

The present inventors have also identified the following cDNA clones related to portions of SEQ ID NO:3: HCE4D69FP02 (SEQ ID NO:42), HIBDB45F (SEQ ID NO:43), HKIXH64R (SEQ ID NO:44), HIBDB45R (SEQ ID NO: 19), HCE3Z95R (SEQ ID NO:45), HTLEQ90R (SEQ ID NO:46), HMWEF45R (SEQ ID NO:47), HTOFC34RA (SEQ ID NO:48), HHFDI20R (SEQ ID NO:49), HMSHY47R (SEQ ID NO:50), HCESF90R (SEQ ID NO:51), HMCAO46R (SEQ ID NO:52), HTTAQ67R (SEQ ID NO:53), HFKCF19F (SEQ ID NO:54), HMCAS31R (SEQ ID NO:55), HMWGP26R (SEQ ID NO:56), HLHTP36R (SEQ ID NO:57), HE8AN11R (SEQ ID NO:58), HEONN73R (SEQ ID NO:59), HBNBG53R (SEQ ID NO:60), and HMSCH94R (SEQ ID NO:61).

The following public ESTs, which relate to portions of the sequence shown in SEQ ID NO:3, have also been identified: D67076 (SEQ ID NO:20), AB001735 (SEQ ID NO:21), AB005287 (SEQ ID NO:62), X87619 (SEQ ID NO:63), X14787 (SEQ ID NO:22), X04665 (SEQ ID NO:24), M87276 (SEQ ID NO:64), M62458 (SEQ ID NO:65), AB002364 (SEQ ID NO:66), AB005297 (SEQ ID NO:67), X69161 (SEQ ID NO:68), X16619 (SEQ ID NO:69), I36448 (SEQ ID NO:70), L12260 (SEQ ID NO:71), 136352 (SEQ ID NO:72), X15898 (SEQ ID NO:73), I07789 (SEQ ID NO:74), I08144 (SEQ ID NO:75) U31814 (SEQ ID NO:76), AF001444 (SEQ ID NO:77), AI400905 (SEQ ID NO:94), AI378857 (SEQ ID NO:95), AA635657 (SEQ ID NO:96), AI464076 (SEQ ID NO:97), CO₆₅₇₈ (SEQ ID NO:98), AA855532 (SEQ ID NO:99), H11881 (SEQ ID NO: 100), AA350801 (SEQ ID NO: 101), and AA350802 (SEQ ID NO: 102).

In specific embodiments, the polynucleotides of the invention are less than 300 kb, 200 kb, 100 kb, 50 kb, 15 kb, 10 kb, or 7.5 kb in length. In a further embodiment, polynucleotides of the invention comprise at least 15 contiguous nucleotides of METH1 or METH2 coding sequence, but do not comprise all or a portion of any METH1 or METH2 intron. In another embodiment, the nucleic acid comprising METH1 or METH2 coding sequence does not contain coding sequences of a genomic flanking gene (i.e., 5′ or 3′ to the METH1 or METH2 gene in the genome).

In another aspect, the invention provides an isolated nucleic acid molecule comprising a polynucleotide which hybridizes under stringent hybridization conditions to a portion of the polynucleotide in a nucleic acid molecule of the invention described above, for instance, the cDNA clones contained in ATCC Deposit No. 209581; ATCC Deposit No. 209582; or ATCC Deposit No. PTA 1478. By “stringent hybridization conditions” is intended overnight incubation at 42° C. in a solution comprising: 50% formamide, 5× SSC (750 mM NaCl, 75 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1× SSC at about 65° C.

By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30, 40, 50, 60 or 70 nt of the reference polynucleotide. These are useful as diagnostic probes and primers as discussed above and in more detail below.

By a portion of a polynucleotide of “at least 20 nt in length,” for example, is intended 20 or more contiguous nucleotides from the nucleotide sequence of the reference polynucleotide (e.g., the deposited cDNAs or the nucleotide sequence as shown in SEQ ID NO:1 or SEQ ID NO:3). Of course, a polynucleotide which hybridizes only to a poly A sequence (such as the 3′ terminal poly(A) tract of the METH1 or METH2 cDNA shown in SEQ ID NO:1 and SEQ ID NO:3, respectively) or to a complementary stretch of T (or U) resides, would not be included in a polynucleotide of the invention used to hybridize to a portion of a nucleic acid of the invention, since such a polynucleotide would hybridize to any nucleic acid molecule containing a poly (A) stretch or the complement thereof (e.g., practically any double-stranded cDNA clone).

Also contemplated are nucleic acid molecules that hybridize to the METH1 or METH2 polynucleotides at moderately high stringency hybridization conditions. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 3° C. in a solution comprising 6× SSPE (20× SSPE=3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1× SSPE, 0.1% SDS. In addition, to achieve even lower stringency, washes performed following stringent hybridization can be done at higher salt concentrations (e.g. 5× SSC).

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Of course, a polynucleotide which hybridizes only to polyA+ sequences (such as any 3′ terminal polyA+ tract of a cDNA shown in the sequence listing), or to a complementary stretch of T (or U) residues, would not be included in the definition of “polynucleotide,” since such a polynucleotide would hybridize to any nucleic acid molecule containing a poly (A) stretch or the complement thereof (e.g., practically any double-stranded cDNA clone).

The METH1 or METH2 polynucleotide can be composed of any polyribonucleotide or polydeoxribonucleotide, which may be unmodified RNA or DNA or modified RNA or DNA. For example, METH1 or METH2 polynucleotides can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, the METH1 or METH2 polynucleotides can be composed of triple-stranded regions comprising RNA or DNA or both RNA and DNA. METH1 or METH2 polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms.

“SEQ ID NO: 1” refers to a METH1 polynucleotide sequence while “SEQ ID NO:2” refers to a METH1 polypeptide sequence. “SEQ ID NO:3” refers to a METH2 polynucleotide sequence while “SEQ ID NO:4” refers to a METH2 polypeptide sequence.

As indicated, nucleic acid molecules of the present invention which encode a METH1 or METH2 polypeptide may include, but are not limited to, those encoding the amino acid sequence of the mature polypeptide, by itself; the coding sequence for the mature polypeptide and additional sequences, such as those encoding the leader or secretory sequence, such as a pre-, or pro- or prepro-protein sequence; the coding sequence of the mature polypeptide, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; an additional coding sequence which codes for additional amino acids, such as those which provide additional functionalities. Thus, the sequence encoding the polypeptide may be fused to a marker sequence, such as a sequence encoding a peptide which facilitates purification of the fused polypeptide. In certain preferred embodiments of this aspect of the invention, the marker amino acid sequence is a hexa-histidine peptide, such as the tag provided in a pQE vector (Qiagen, Inc.), among others, many of which are commercially available. As described in Gentz et al., Proc. Natl. Acad. Sci. USA 86:821-824 (1989), for instance, hexa-histidine provides for convenient purification of the fusion protein. The “HA” tag is another peptide useful for purification which corresponds to an epitope derived from the influenza hemagglutinin protein, which has been described by Wilson et al., Cell 37:767-778 (1984). As discussed below, other such fusion proteins include the METH1 or METH2 fused to Fc at the - or C-terminus. Other fusion proteins include METH1 or METH2 fused to Flag at the - or C-terminus. Other fusion proteins include METH1 fragments or METH2 fragments fused to Flag or Fc at the - or C-terminus. Particularly preferred are fragments of METH1 or METH2, such as H541-Q894, M1-P799, F236-E614, or K801-Q950 of SEQ ID NO:2, fused to Fc or Flag at the - or C-terminus.

As stated above, METH1 or METH 2 may be fused with the FLAG polypeptide sequence (see U.S. Pat. No. 4,851,341; see also Hopp et al., Bio/Technology 6:1204, 1988). The FLAG polypeptide sequence is highly antigenic and provides an epitope for binding by a specific monoclonal antibody, enabling rapid purification of the expressed recombinant protein. This sequence is also specifically cleaved by bovine mucosal enterokinase at the residue immediately following the Asp-Lys pairing.

The present invention further relates to variants of the nucleic acid molecules of the present invention, which encode portions, analogs or derivatives of the METH1 or METH2 protein. Variants may occur naturally, such as a natural allelic variant. By an “allelic variant” is intended one of several alternate forms of a gene occupying a given locus on a chromosome of an organism. Lewin, B., ed., Genes II, John Wiley & Sons, New York (1985). Non-naturally occurring variants may be produced using art-known mutagenesis techniques.

Such variants include those produced by nucleotide substitutions, deletions or additions, which may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the METH1 or METH2 protein or portions thereof. Also especially preferred in this regard are conservative substitutions.

Further embodiments of the invention include isolated nucleic acid molecules comprising a polynucleotide having a nucleotide sequence at least 80% identical, and more preferably at least 85%, 90%, 91%, 92%, 93%, 94%, 95%, 96%, 97%, 98% or 99% identical to: a nucleotide sequence encoding the polypeptide having the amino acid sequence in SEQ ID NO:2; a nucleotide sequence encoding the polypeptide having the amino acid sequence in SEQ ID NO:2, but lacking the N-terminal methionine; a nucleotide sequence encoding the polypeptide having the amino acid sequence at positions from about 29 to about 950 in SEQ ID NO:2; a nucleotide sequence encoding the polypeptide having the amino acid sequence at position from about 30 to about 950 in SEQ ID NO:2; a nucleotide sequence encoding the polypeptide having the amino acid sequence encoded by the cDNA clone contained in ATCC Deposit No. 209581; a nucleotide sequence encoding the mature METH1 polypeptide having the amino acid sequence encoded by the cDNA clone contained in ATCC Deposit No. 209581; a nucleotide sequence encoding amino acids 235 to 459 in SEQ ID NO:2 (the metalloprotease domain of METH1); a nucleotide sequence encoding amino acids 460 to 544 in SEQ ID NO:2 (the disintegrin domain of METH1); a nucleotide sequence encoding amino acids 545 to 598 in SEQ ID NO:2 (the first TSP-like domain of METH1); a nucleotide sequence encoding amino acids 841 to 894 in SEQ ID NO:2 (the second TSP-like domain of METH1); a nucleotide sequence encoding amino acids 895 to 934 in SEQ ID NO:2 (the third TSP-like domain of METH1); a nucleotide sequence encoding amino acids 536 to 613 in SEQ ID NO:2; a nucleotide sequence encoding amino acids 549 to 563 in SEQ ID NO:2; a nucleotide sequence encoding the polypeptide having the amino acid sequence in SEQ ID NO:4; a nucleotide sequence encoding the polypeptide having the amino acid sequence in SEQ ID NO:4, but lacking the N-terminal methionine; a nucleotide sequence encoding the polypeptide having the amino acid sequence at positions from about 24 to about 890 in SEQ ID NO:4; a nucleotide sequence encoding the polypeptide having the amino acid sequence at positions from about 112 to about 890 in SEQ ID NO:4; a nucleotide sequence encoding the polypeptide having the amino acid sequence encoded by the cDNA clone contained in ATCC Deposit No. 209582 or PTA 1478; a nucleotide sequence encoding the mature METH2 polypeptide having the amino acid sequence encoded by the cDNA clone contained in ATCC Deposit No. 209582 or PTA 1478; a nucleotide sequence encoding amino acids 214 to 439 in SEQ ID NO:4 (the metalloprotease domain of METH2); a nucleotide sequence encoding amino acids 440 to 529 in SEQ ID NO:4 (the disintegrin domain of METH2); a nucleotide sequence encoding amino acids 530 to 583 in SEQ ID NO:4 (the first TSP-like domain of METH2); a nucleotide sequence encoding amino acids 837 to 890 in SEQ ID NO:4 (the second TSP-like domain of METH2); a nucleotide sequence encoding amino acids 280 to 606 in SEQ ID NO:4; a nucleotide sequence encoding amino acids 529 to 548 in SEQ ID NO:4; or a nucleotide sequence complementary to any of the above nucleotide sequences.

By a polynucleotide having a nucleotide sequence at least, for example, 95% “identical” to a reference nucleotide sequence encoding a METH1 or METH2 polypeptide is intended that the nucleotide sequence of the polynucleotide is identical to the reference sequence except that the polynucleotide sequence may include up to five point mutations per each 100 nucleotides of the reference nucleotide sequence encoding the METH1 or METH2 polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 80%, 85%, 90%, 91%, 92%, 93%, 94%, 95%, 96%, 97%, 98% or 99% identical to, for instance, the nucleotide sequence shown in SEQ ID NO: 1 or SEQ ID NO:3 or to the nucleotide sequence of the deposited cDNA clones can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

A preferred method for determining the best overall match between a query sequence (a sequence of the present invention) and a subject sequence, also referred to as a global sequence alignment, can be determined using the FASTDB computer program based on the algorithm of Brutlag et al., Comp. Appl. Biosci. 6:237-245 (1990). In a sequence alignment, the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty=1, Joining Penalty=30, Randomization Group Length=O, Cutoff Score=1, Gap Penalty=5, Gap Size Penalty=0.05, Window Size=500 or the length of the subject nucleotide sequence, whichever is shorter.

If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by the results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence are calculated for the purposes of manually adjusting the percent identity score.

For example, a 90 base subject sequence is aligned to a 100 base query sequence to determine percent identity. The deletions occur at the 5′ end of the subject sequence and, therefore, the FASTDB alignment does not show a match/alignment of the first 10 bases at the 5′ end. The 10 unpaired bases represent 10% of the sequence (number of bases at the 5′ and 3′ ends not matched/total number of bases in the query sequence), so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal, so that there are no bases on the 5′ or 3′ ends of the subject sequence which are not matched/aligned with the query. In this case, the percent identity calculated by FASTDB is not manually corrected. Once again, only bases 5′ and 3′ of the subject sequence which are not matched/aligned with the query sequence are manually corrected for. No other manual corrections are to be made for the purposes of the present invention.

The present application is directed to nucleic acid molecules at least 80%, 85%, 90%, 91%, 92%, 93%, 94%, 95%, 96%, 97%, 98% or 99% identical to the nucleic acid sequence shown in SEQ ID NO:1 or SEQ ID NO:3 or to the nucleic acid sequence of the deposited cDNAs, irrespective of whether they encode a polypeptide having METH1 or METH2 activity. This is because even where a particular nucleic acid molecule does not encode a polypeptide having METH1 or METH2 activity, one of skill in the art would still know how to use the nucleic acid molecule, for instance, as a hybridization probe or a polymerase chain reaction (PCR) primer. Uses of the nucleic acid molecules of the present invention that do not encode a polypeptide having METH1 or METH2 activity include, inter alia, (1) isolating the METH1 or METH2 gene or allelic variants thereof in a cDNA library; (2) in situ hybridization (e.g., “FISH”) to metaphase chromosomal spreads to provide precise chromosomal location of the METH1 or METH2 gene, as described in Verma et al., Human Chromosomes: A Manual of Basic Techniques, Pergamon Press, New York (1988); and (3) Northern Blot analysis for detecting METH1 or METH2 mRNA expression in specific tissues.

Preferred, however, are nucleic acid molecules having sequences at least 80%, 85%, 90%, 91%, 92%, 93%, 94%, 95%, 96%, 97%, 98% or 99% identical to the nucleic acid sequence shown in SEQ ID NO: 1 or SEQ ID NO:3 or to a nucleic acid sequence of the deposited cDNAs which do, in fact, encode a polypeptide having METH1 or METH2 protein activity. By “a polypeptide having METH1 activity” is intended polypeptides exhibiting METH1 activity in a particular biological assay. For example, METH1 protein activity can be measured using the chorioallantoic membrane assay (Iruela-Arispe et al., Thrombosis and Haemostasis 78(1):672-677 (1997)) or the cornea pocket assay (Tolsma et al., J. Cell. Biol. 122:497-511 (1993)), both described in Example 4, below. By “a polypeptide having METH2 activity” is intended polypeptides exhibiting METH2 activity in a particular biological assay. For example, METH2 protein activity can also be measured using the chorioallantoic membrane assay (Iruela-Arispe et al., Thrombosis and Haemostasis 78(1):672-677 (1997)) or the cornea pocket assay (Tolsma et al., J. Cell. Biol. 122:497-511 (1993)), both described in Example 4, below.

Briefly, in the chorioallantoic assay, the potentially anti-angiogenic compound of interest is added to type I collagen pellets (Vitrogen), along with an angiogenic growth factor, such as bFGF. The samples are mixed and placed onto nylon meshes, and allowed to polymerize. After polymerization is complete, the meshes are placed onto the chorioallantoic membrane of 12 day old chick embryos and placed at 37° C. for 24 hours. The embryos are then injected with a fluorescent agent, such as FrFC-dextran, and the meshes are fixed and mounted for observation under a fluorescent microscope.

In the cornea pocket assay, hydron pellets containing the compound of interest and an angiogenic growth factor, such as bFGF, are implanted 1 to 2 mm from the limbus of the cornea of rats or mice. Response is examined after a period of time, for example 5 days. The extent of angiogenesis is evaluated by measuring the capillaries migrating from the limb of the cornea.

Of course, due to the degeneracy of the genetic code, one of ordinary skill in the art will immediately recognize that a large number of the nucleic acid molecules having a sequence at least 80%, 85%, 90%, 91%, 92%, 93%, 94%, 95%, 96%, 97%, 98% or 99% identical to a nucleic acid sequence of the deposited cDNAs or a nucleic acid sequence shown in SEQ ID NO:1 or SEQ ID NO:3 will encode a polypeptide “having METH1 or METH2 protein activity.” In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having METH1 or METH2 protein activity. This is because the skilled artisan is fully aware of amino acid substitutions that are either less likely or not likely to significantly effect protein function (e.g., replacing one aliphatic amino acid with a second aliphatic amino acid).

In particular, there are provided METH2 nucleic acids with one or more of the following nucleic acid substitutions and/or deletions: “C” substituted for “T” at position 3; “C” substituted for “T” at position 32; “C” substituted for “T” at position 37; “TGC” at positions 65-67 deleted; “C” substituted for “T” at position 199; “C” substituted for “T” at position 303; “C” substituted for “T” at position 306; “C” substituted for “T” at position 309; “C” substituted for “T” at position 950; “C” substituted for “G” at position 1292; “C” substituted for “T” at position 1577; and/or “G” substituted for “A” at position 2377. Likewise, there are provided METH2 polypeptides with one or more of the following amino acid substitutions and/or deletions: “L” substituted for “F” at position 2; “P” substituted for “L” at position 12; “L” substituted for “F” at position 14; “L” at position 23 deleted; “P” substituted for “L” at position 318; “A” substituted for “G” at position 432; “A” substituted for “V” at position 527; and/or “A” substituted for “T” at position 794.

For example, guidance concerning how to make phenotypically silent amino acid substitutions is provided in Bowie, J. U. et al., “Deciphering the Message in Protein Sequences: Tolerance to Amino Acid Substitutions,” Science 247:1306-1310 (1990), wherein the authors indicate that proteins are surprisingly tolerant of amino acid substitutions.

Vectors and Host Cells

The present invention also relates to vectors which include the isolated DNA molecules of the present invention, host cells which are genetically engineered with the recombinant vectors, and the production of METH1 or METH2 polypeptides or fragments thereof by recombinant techniques. Cell-free translation systems can also be employed to produce such proteins using RNAs derived from the DNA constructs of the present invention.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The DNA insert should be operatively linked to an appropriate promoter, such as the phage lambda PL promoter, the E. coli lac, trp and tac promoters, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the mature transcripts expressed by the constructs will preferably include a translation initiating at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, 293, and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc.; pBluescript vectors, Phagescript vectors, pNH8A, pNH16a, pNH18A, pNH46A, available from Stratagene Cloning Systems, Inc.; and ptrc99a, pKK223-3, pKK233-3, pDR540, pRIT5 available from Pharmacia Biotech, Inc. Among preferred eukaryotic vectors are pWLNEO, pSV2CAT, pOG44, pXT1 and pSG available from Stratagene; and pSVK3, pBPV, pMSG and pSVL available from Pharmacia. Preferred expression vectors for use in yeast systems include, but are not limited to pYES2, pYD1, pTEF1/Zeo, pYES2/GS, pPICZ, pGAPZ, pGAPZalph, pPIC9, pPIC3.5, pHIL-D2, pHIL-Si, pPIC3.5K, pPIC9K, and PA0815 (all available from Invitrogen, Carlbad, Calif.). Other suitable vectors will be readily apparent to the skilled artisan.

In addition to the use of expression vectors in the practice of the present invention, the present invention further includes novel expression vectors comprising operator and promoter elements operatively linked to nucleotide sequences encoding a protein of interest. One example of such a vector is pHE4-5 which is described in detail below.

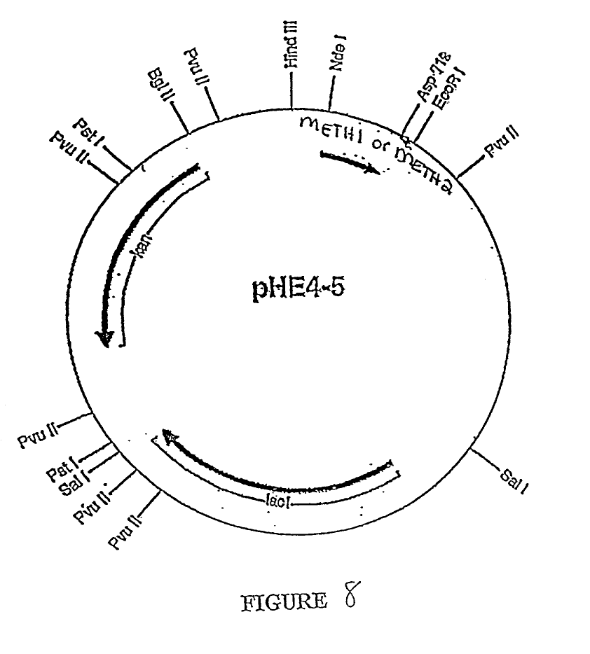

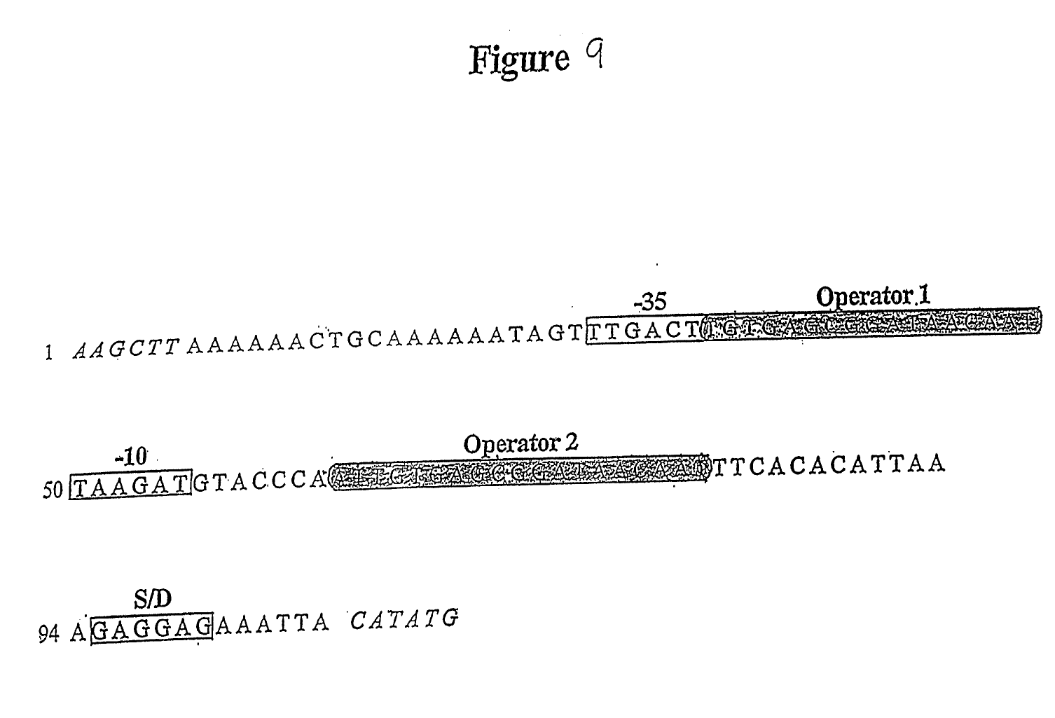

As summarized in FIGS. 8 and 9, components of the pHE4-5 vector (SEQ ID NO:12) include: 1) a neomycinphosphotransferase gene as a selection marker, 2) an E. coli origin of replication, 3) a T5 phage promoter sequence, 4) two lac operator sequences, 5) a Shine-Delgarno sequence, 6) the lactose operon repressor gene (lacIq). The origin of replication (oriC) is derived from pUC19 (LTI, Gaithersburg, Md.). The promoter sequence and operator sequences were made synthetically. Synthetic production of nucleic acid sequences is well known in the art. CLONTECH 95/96 Catalog, pages 215-216, CLONTECH, 1020 East Meadow Circle, Palo Alto, Calif. 94303. A nucleotide sequence encoding METH1 (SEQ ID NO:2) or METH2 (SEQ ID NO:4), is operatively linked to the promoter and operator by inserting the nucleotide sequence between the NdeI and Asp718 sites of the pHE4-5 vector.

As noted above, the pHE4-5 vector contains a lacIq gene. LacIq is an allele of the lacI gene which confers tight regulation of the lac operator. Amann, E. et al., Gene 69:301-315 (1988); Stark, M., Gene 51:255-267 (1987). The lacIq gene encodes a repressor protein which binds to lac operator sequences and blocks transcription of down-stream (i.e., 3′) sequences. However, the lacIq gene product dissociates from the lac operator in the presence of either lactose or certain lactose analogs, e.g., isopropyl B-D-thiogalactopyranoside (IPTG). METH1 or METH2 thus is not produced in appreciable quantities in uninduced host cells containing the pHE4-5 vector. Induction of these host cells by the addition of an agent such as IPTG, however, results in the expression of the METH1 or METH2 coding sequence.

The promoter/operator sequences of the pHE4-5 vector (SEQ ID NO:13) comprise a T5 phage promoter and two lac operator sequences. One operator is located 5′ to the transcriptional start site and the other is located 3′ to the same site. These operators, when present in combination with the lacIq gene product, confer tight repression of down-stream sequences in the absence of a lac operon inducer, e.g., IPTG. Expression of operatively linked sequences located down-stream from the lac operators may be induced by the addition of a lac operon inducer, such as IPTG. Binding of a lac inducer to the lacIq proteins results in their release from the lac operator sequences and the initiation of transcription of operatively linked sequences. Lac operon regulation of gene expression is reviewed in Devlin, T., TEXTBOOK OF BIOCHEMISTRY WITH CLINICAL CORRELATIONS, 4th Edition (1997), pages 802-807.

The pEIE4 series of vectors contain all of the components of the pHE4-5 vector except for the METH1 or METH2 coding sequence. Features of the pHE4 vectors include optimized synthetic T5 phage promoter, lac operator, and Shine-Delgarno sequences. Further, these sequences are also optimally spaced so that expression of an inserted gene may be tightly regulated and high level of expression occurs upon induction.

Among known bacterial promoters suitable for use in the production of proteins of the present invention include the E. coli lacI and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous Sarcoma Virus (RSV), and metallothionein promoters, such as the mouse metallothionein-I promoter.

The pHE4-5 vector also contains a Shine-Delgarno sequence 5′ to the AUG initiation codon. Shine-Delgarno sequences are short sequences generally located about 10 nucleotides up-stream (ie., 5′) from the AUG initiation codon. These sequences essentially direct prokaryotic ribosomes to the AUG initiation codon.

Thus, the present invention is also directed to expression vectors useful for the production of the proteins of the present invention. This aspect of the invention is exemplified by the pHE⁴-5 vector (SEQ ID NO: 12).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986). It is specifically contemplated that METH1 and/or METH2 polypeptides may in fact be expressed by a host cell lacking a recombinant vector.

The polypeptide may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to solubilize proteins. For example, EP-A-O 464 533 (Canadian counterpart 2045869) discloses fusion proteins comprising various portions of constant region of immunoglobin molecules together with another human protein or part thereof. In many cases, the Fc part in a fusion protein is thoroughly advantageous for use in therapy and diagnosis and thus results, for example, in improved pharmacokinetic properties EP-A 0232 262). On the other hand, for some uses it would be desirable to be able to delete the Fc part after the fusion protein has been expressed, detected and purified in the advantageous manner described. This is the case when the Fc portion proves to be a hindrance to use in therapy and diagnosis, for example when the fusion protein is to be used as an antigen for immunizations. In drug discovery, for example, human proteins, such as the hIL5-receptor, have been fused with Fc portions for the purpose of high-throughput screening assays to identify antagonists of hIL-5. See, D. Bennett et al., J. Mol. Recognition 8:52-58 (1995) and K. Johanson et al., J. of Biol. Chem. 270(16):9459-9471 (1995).

The METH1 or METH2 protein can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification.

METH1 and/or METH2 polypeptides, and preferably the secreted form, can also be recovered from: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect, and mammalian cells. Depending upon the host employed in a recombinant production procedure, the METH1 and/or METH2 polypeptides maybe glycosylated or may be non-glycosylated. In addition, METH1 and/or METH2 polypeptides may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

In one embodiment, the yeast Pichia pastoris is used to express METH1 and/or METH2 protein in a eukaryotic system. Pichia pastoris is a methylotrophic yeast which can metabolize methanol as its sole carbon source. A main step in the methanol metabolization pathway is the oxidation of methanol to formaldehyde using O₂. This reaction is catalyzed by the enzyme alcohol oxidase. In order to metabolize methanol as its sole carbon source, Pichia pastoris must generate high levels of alcohol oxidase due, in part, to the relatively low affinity of alcohol oxidase for O₂. Consequently, in a growth medium depending on methanol as a main carbon source, the promoter region of one of the two alcohol oxidase genes (AOX1) is highly active. In the presence of methanol, alcohol oxidase produced from the AOX1 gene comprises up to approximately 30% of the total soluble protein in Pichia pastoris. See, Ellis, S. B., et al., Mol. Cell Biol. 5:1111-21 (1985); Koutz, P. J, et al., Yeast 5:167-77 (1989); Tschopp, J. F., et al., Nucl. Acids Res. 15:3859-76 (1987). Thus, a heterologous coding sequence, such as, for example, a METH1 and/or METH2 polynucleotide of the present invention, under the transcriptional regulation of all or part of the AOX1 regulatory sequence is expressed at exceptionally high levels in Pichia yeast grown in the presence of methanol.

In one example, the plasmid vector pPIC9K is used to express DNA encoding a METH1 and/or METH2 polypeptide of the invention, as set forth herein, in a Pichea yeast system essentially as described in “Pichia Protocols: Methods in Molecular Biology,” D. R. Higgins and J. Cregg, eds. The Humana Press, Totowa, N.J., 1998. This expression vector allows expression and secretion of a METH1 and/or METH2 protein of the invention by virtue of the strong AOX1 promoter linked to the Pichia pastoris alkaline phosphatase (PHO) secretory signal peptide (i.e., leader) located upstream of a multiple cloning site.

Many other yeast vectors could be used in place of pPIC9K, such as, pYES2, pYD1, pTEF1/Zeo, pYES2/GS, pPICZ, pGAPZ, pGAPZalpha, pPIC9, pPIC3.5, pHIL-D2, pHIL-S1, pPIC3.5K, and PAO815, as one skilled in the art would readily appreciate, as long as the proposed expression construct provides appropriately located signals for transcription, translation, secretion (if desired), and the like, including an in-frame AUG as required.

In another embodiment, high-level expression of a heterologous coding sequence, such as, for example, a METH1 and/or METH2 polynucleotide of the present invention, may be achieved by cloning the heterologous polynucleotide of the invention into an expression vector such as, for example, pGAPZ or pGAPZalpha, and growing the yeast culture in the absence of methanol.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., METH1 and/or METH2 coding sequence), and/or to include genetic material (e.g., heterologous polynucleotide sequences) that is operably associated with METH1 and/or METH2 polynucleotides of the invention, and which activates, alters, and/or amplifies endogenous METH1 and/or METH2 polynucleotides. For example, techniques known in the art may be used to operably associate heterologous control regions (e.g., promoter and/or enhancer) and endogenous METH1 and/or METH2 polynucleotide sequences via homologous recombination, resulting in the formation of a new transcription unit (see, e.g., U.S. Pat. No. 5,641,670, issued Jun. 24, 1997; U.S. Pat. No. 5,733,761, issued Mar. 31, 1998; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435438 (1989), the disclosures of each of which are incorporated by reference in their entireties).

Polypeptides of the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or maybe non-glycosylated. In addition, polypeptides of the invention may also include an initial modified methionine residue, in some cases as a result of host-mediated processes.

In addition, polypeptides of the invention can be chemically synthesized using techniques known in the art (e.g., see Creighton, 1983, Proteins: Structures and Molecular Principles, W. H. Freeman & Co., N.Y., and Hunkapiller, M., et al., 1984, Nature 310:105-111). For example, a peptide corresponding to a fragment of the METH1 and/or METH2 polypeptides of the invention can be synthesized by use of a peptide synthesizer. Furthermore, if desired, nonclassical amino acids or chemical amino acid analogs can be introduced as a substitution or addition into the METH1 and/or ?METH2 polypeptide sequence. Non-classical amino acids include, but are not limited to, to the D-isomers of the common amino acids, 2,4-diaminobutyric acid, α-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, omithine, norleucine, norvaline, hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general. Furthermore, the amino acid can be D (dextrorotary) or L (levorotary).

Non-naturally occurring variants may be produced using art-known mutagenesis techniques, which include, but are not limited to oligonucleotide mediated mutagenesis, alanine scanning, PCR mutagenesis, site directed mutagenesis (see, e.g., Carter et al., Nucl. Acids Res. 13:4331 (1986); and Zoller et al., Nucl. Acids Res. 10:6487 (1982)), cassette mutagenesis (see, e.g., Wells et al., Gene 34:315 (1985)), restriction selection mutagenesis (see, e.g., Wells et al., Philos. Trans. R. Soc. London SerA 317:415 (1986)).

The invention encompasses METH1 and/or METH2 polypeptides which are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH₄; acetylation, formylation, oxidation, reduction; metabolic synthesis in the presence of tunicamycin; etc.

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of procaryotic host cell expression. The polypeptides may also be modified with a detectable label, such as an enzymatic, fluorescent, isotopic or affinity label to allow for detection and isolation of the protein.

Also provided by the invention are chemically modified derivatives of METH1 and/or METH2 which may provide additional advantages such as increased solubility, stability and circulating time of the polypeptide, or decreased immunogenicity (see U.S. Pat. No. 4,179,337). The chemical moieties for derivitization may be selected from water soluble polymers such as polyethylene glycol, ethylene glycol/propylene glycol copolymers, carboxymethylcellulose, dextran, polyvinyl alcohol and the like. The polypeptides may be modified at random positions within the molecule, or at predetermined positions within the molecule and may include one, two, three or more attached chemical moieties.

The polymer may be of any molecular weight, and may be branched or unbranched. For polyethylene glycol, the preferred molecular weight is between about 1 kDa and about 100 kDa (the term “about” indicating that in preparations of polyethylene glycol, some molecules will weigh more, some less, than the stated molecular weight) for ease in handling and manufacturing. Other sizes may be used, depending on the desired therapeutic profile (e.g., the duration of sustained release desired, the effects, if any on biological activity, the ease in handling, the degree or lack of antigenicity and other known effects of the polyethylene glycol to a therapeutic protein or analog). For example, the polyethylene glycol may have an average molecular weight of about 200, 500, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 25,000, 30,000, 35,000, 40,000, 50,000, 55,000, 60,000, 65,000, 70,000, 75,000, 80,000, 85,000, 90,000, 95,000, or 100,000 kDa.

As noted above, the polyethylene glycol may have a branched structure. Branched polyethylene glycols are described, for example, in U.S. Pat. No. 5,643,575; Morpurgo et al., Appl. Biochem. Biotechnol. 56:59-72 (1996); Vorobjev et al., Nucleosides Nucleotides 18:2745-2750 (1999); and Caliceti et al., Bioconjug. Chem. 10:638-646 (1999), the disclosures of each of which are incorporated herein by reference.

The polyethylene glycol molecules (or other chemical moieties) should be attached to the protein with consideration of effects on functional or antigenic domains of the protein. There are a number of attachment methods available to those skilled in the art, e.g., EP 0 401 384, herein incorporated by reference (coupling PEG to G-CSF), see also Malik et al., Exp. Hematol. 20:1028-1035 (1992) (reporting pegylation of GM-CSF using tresyl chloride). For example, polyethylene glycol may be covalently bound through amino acid residues via a reactive group, such as, a free amino or carboxyl group. Reactive groups are those to which an activated polyethylene glycol molecule may be bound. The amino acid residues having a free amino group may include lysine residues and the N-terminal amino acid residues; those having a free carboxyl group may include aspartic acid residues glutamic acid residues and the C-terminal amino acid residue. Sulfhydryl groups may also be used as a reactive group for attaching the polyethylene glycol molecules. Preferred for therapeutic purposes is attachment at an amino group, such as attachment at the N-terminus or lysine group.

As suggested above, polyethylene glycol may be attached to proteins via linkage to any of a number of amino acid residues. For example, polyethylene glycol can be linked to a proteins via covalent bonds to lysine, histidine, aspartic acid, glutamic acid, or cysteine residues. One or more reaction chemistries may be employed to attach polyethylene glycol to specific amino acid residues (e.g., lysine, histidine, aspartic acid, glutamic acid, or cysteine) of the protein or to more than one type of amino acid residue (e.g., lysine, histidine, aspartic acid, glutamic acid, cysteine and combinations thereof) of the protein.

One may specifically desire proteins chemically modified at the N-terminus. Using polyethylene glycol as an illustration of the present composition, one may select from a variety of polyethylene glycol molecules (by molecular weight, branching, etc.), the proportion of polyethylene glycol molecules to protein (or peptide) molecules in the reaction mix, the type of pegylation reaction to be performed, and the method of obtaining the selected N-terminally pegylated protein. The method of obtaining the N-terminally pegylated preparation (i.e., separating this moiety from other monopegylated moieties if necessary) may be by purification of the N-terminally pegylated material from a population of pegylated protein molecules. Selective proteins chemically modified at the N-terminus modification may be accomplished by reductive alkylation which exploits differential reactivity of different types of primary amino groups (lysine versus the N-terminal) available for derivatization in a particular protein. Under the appropriate reaction conditions, substantially selective derivatization of the protein at the N-terminus with a carbonyl group containing polymer is achieved.

As indicated above, pegylation of the proteins of the invention may be accomplished by any number of means. For example, polyethylene glycol may be attached to the protein either directly or by an intervening linker. Linkerless systems for attaching polyethylene glycol to proteins are described in Delgado et al., Crit. Rev. Thera. Drug Carrier Sys. 9:249-304 (1992); Francis et al., Intern. J. of Hematol. 68:1-18 (1998); U.S. Pat. No. 4,002,531; U.S. Pat. No. 5,349,052; WO 95/06058; and WO 98/32466, the disclosures of each of which are incorporated herein by reference.

One system for attaching polyethylene glycol directly to amino acid residues of proteins without an intervening linker employs tresylated MPEG, which is produced by the modification of monmethoxy polyethylene glycol (MPEG) using tresylchloride (ClSO₂CH₂CF₃). Upon reaction of protein with tresylated MPEG, polyethylene glycol is directly attached to amine groups of the protein. Thus, the invention includes protein-polyethylene glycol conjugates produced by reacting proteins of the invention with a polyethylene glycol molecule having a 2,2,2-trifluoreothane sulphonyl group.

Polyethylene glycol can also be attached to proteins using a number of different intervening linkers. For example, U.S. Pat. No. 5,612,460, the entire disclosure of which is incorporated herein by reference, discloses urethane linkers for connecting polyethylene glycol to proteins. Protein-polyethylene glycol conjugates wherein the polyethylene glycol is attached to the protein by a linker can also be produced by reaction of proteins with compounds such as MPEG-succinimidylsuccinate, MPEG activated with 1,1′-carbonyldiimidazole, MPEG-2,4,5-trichloropenylcarbonate, MPEG-p-nitrophenolcarbonate, and various MPEG-succinate derivatives. A number additional polyethylene glycol derivatives and reaction chemistries for attaching polyethylene glycol to proteins are described in WO 98/32466, the entire disclosure of which is incorporated herein by reference. Pegylated protein products produced using the reaction chemistries set out herein are included within the scope of the invention.

The number of polyethylene glycol moieties attached to each protein of the invention (i.e., the degree of substitution) may also vary. For example, the pegylated proteins of the invention may be linked, on average, to 1, 2, 3, 4, 5,6,7, 8, 9, 10, 12, 15, 17, 20, or more polyethylene glycol molecules. Similarly, the average degree of substitution within ranges such as 1-3, 2-4, 3-5, 4-6, 5-7, 6-8, 7-9, 8-10, 9-11, 10-12, 11-13, 12-14, 13-15, 14-16, 15-17, 16-18, 17-19, or 18-20 polyethylene glycol moieties per protein molecule. Methods for determining the degree of substitution are discussed, for example, in Delgado et al., Crit. Rev. Thera. Drug Carrier Sys. 9:249-304 (1992).

The METH1 and/or METH2 polypeptides of the invention may be in monomers or multimers (i.e., dimers, trimers, tetramers and higher multimers). Accordingly, the present invention relates to monomers and multimers of the METH1 and/or METH2 polypeptides of the invention, their preparation, and compositions (preferably, pharmaceutical compositions) containing them. In specific embodiments, the polypeptides of the invention are monomers, dimers, trimers or tetramers. In additional embodiments, the multimers of the invention are at least dimers, at least trimers, or at least tetramers.

Multimers encompassed by the invention may be homomers or heteromers. As used herein, the term homomer, refers to a multimer containing only METH1 and/or METH2 polypeptides of the invention (including METH1 and/or METH2 fragments, variants, splice variants, and fusion proteins, as described herein). These homomers may contain METH1 and/or METH2 polypeptides having identical or different amino acid sequences. In a specific embodiment, a homomer of the invention is a multimer containing only METH1 and/or METH2 polypeptides having an identical amino acid sequence. In another specific embodiment, a homomer of the invention is a multimer containing METH1 and/or METH2 polypeptides having different amino acid sequences. In specific embodiments, the multimer of the invention is a homodimer (e.g., containing METH1 and/or METH2 polypeptides having identical or different amino acid sequences) or a homotrimer (e.g., containing METH1 and/or METH2 polypeptides having identical and/or different amino acid sequences). In additional embodiments, the homomeric multimer of the invention is at least a homodimer, at least a homotrimer, or at least a homotetramer.

As used herein, the term heteromer refers to a multimer containing one or more heterologous polypeptides (i.e., polypeptides of different proteins) in addition to the METH1 and/or METH2 polypeptides of the invention. In a specific embodiment, the multimer of the invention is a heterodimer, a heterotrimer, or a heterotetramer. In additional embodiments, the homomeric multimer of the invention is at least a homodimer, at least a homotrimer, or at least a homotetramer.

Multimers of the invention may be the result of hydrophobic, hydrophilic, ionic and/or covalent associations and/or may be indirectly linked by, for example, liposome formation. Thus, in one embodiment, multimers of the invention, such as, for example, homodimers or homotrimers, are formed when polypeptides of the invention contact one another in solution. In another embodiment, heteromultimers of the invention, such as, for example, heterotrimers or heterotetramers, are formed when polypeptides of the invention contact antibodies to the polypeptides of the invention (including antibodies to the heterologous polypeptide sequence in a fusion protein of the invention) in solution. In other embodiments, multimers of the invention are formed by covalent associations with and/or between the METH1 and/or METH2 polypeptides of the invention. Such covalent associations may involve one or more amino acid residues contained in the polypeptide sequence (e.g., that recited in SEQ ID NO:2 or 4, or contained in the polypeptide encoded by either the clone HATCK89 or the clones deposited as ATCC Deposit No.209581 or 209582 or PTA 1478). In one instance, the covalent associations are cross-linking between cysteine residues located within the polypeptide sequences which interact in the native (i.e., naturally occurring) polypeptide. In another instance, the covalent associations are the consequence of chemical or recombinant manipulation. Alternatively, such covalent associations may involve one or more amino acid residues contained in the heterologous polypeptide sequence in a METH1 and/or METH2 fusion protein. In one example, covalent associations are between the heterologous sequence contained in a fusion protein of the invention (see, e.g., U.S. Pat. No. 5,478,925). In a specific example, the covalent associations are between the heterologous sequence contained in a METH1 and/or METH2-Fc fusion protein of the invention (as described herein). In another specific example, covalent associations of fusion proteins of the invention are between heterologous polypeptide sequence from another Fibroblast Growth Factor family member that is capable of forming covalently associated multimers, such as for example, oseteoprotegerin (see, e.g., International Publication No. WO 98/49305, the contents of which are herein incorporated by reference in its entirety). In another embodiment, two or more polypeptides of the invention are joined through peptide linkers. Examples include those peptide linkers described in U.S. Pat. No. 5,073,627 (hereby incorporated by reference). Proteins comprising multiple polypeptides of the invention separated by peptide linkers may be produced using conventional recombinant DNA technology.

Another method for preparing multimer polypeptides of the invention involves use of polypeptides of the invention fused to a leucine zipper or isoleucine zipper polypeptide sequence. Leucine zipper and isoleucine zipper domains are polypeptides that promote multimerization of the proteins in which they are found. Leucine zippers were originally identified in several DNA-binding proteins (Landschulz et al., Science 240:1759, (1988)), and have since been found in a variety of different proteins. Among the known leucine zippers are naturally occurring peptides and derivatives thereof that dimerize or trimerize. Examples of leucine zipper domains suitable for producing soluble multimeric proteins of the invention are those described in PCT application WO 94/10308, hereby incorporated by reference. Recombinant fusion proteins comprising a polypeptide of the invention fused to a polypeptide sequence that dimerizes or trimerizes in solution are expressed in suitable host cells, and the resulting soluble multimeric fusion protein is recovered from the culture supernatant using techniques known in the art.

Trimeric polypeptides of the invention may offer the advantage of enhanced biological activity. Preferred leucine zipper moieties and isoleucine moieties are those that preferentially form trimers. One example is a leucine zipper derived from lung surfactant protein D (SPD), as described in Hoppe et al. (FEBS Letters 344:191, (1994)) and in U.S. patent application Ser. No. 08/446,922, hereby incorporated by reference. Other peptides derived from naturally occurring trimeric proteins may be employed in preparing trimeric polypeptides of the invention.

In another example, proteins of the invention are associated by interactions between Flag® polypeptide sequence contained in fusion proteins of the invention containing Flag® polypeptide seuqence. In a further embodiment, associations proteins of the invention are associated by interactions between heterologous polypeptide sequence contained in Flag® fusion proteins of the invention and anti-Flag® antibody.

The multimers of the invention may be generated using chemical techniques known in the art. For example, polypeptides desired to be contained in the multimers of the invention may be chemically cross-linked using linker molecules and linker molecule length optimization techniques known in the art (see, e.g., U.S. Pat. No. 5,478,925, which is herein incorporated by reference in its entirety). Additionally, multimers of the invention may be generated using techniques known in the art to form one or more inter-molecule cross-links between the cysteine residues located within the sequence of the polypeptides desired to be contained in the multimer (see, e.g., U.S. Pat. No. 5,478,925, which is herein incorporated by reference in its entirety). Further, polypeptides of the invention may be routinely modified by the addition of cysteine or biotin to the C terminus or N-terminus of the polypeptide and techniques known in the art may be applied to generate multimers containing one or more of these modified polypeptides (see, e.g., U.S. Pat. No. 5,478,925, which is herein incorporated by reference in its entirety). Additionally, techniques known in the art may be applied to generate liposomes containing the polypeptide components desired to be contained in the multimer of the invention (see, e.g., U.S. Pat. No. 5,478,925, which is herein incorporated by reference in its entirety).

Alternatively, multimers of the invention may be generated using genetic engineering techniques known in the art. In one embodiment, polypeptides contained in multimers of the invention are produced recombinantly using fusion protein technology described herein or otherwise known in the art (see, e.g., U.S. Pat. No. 5,478,925, which is herein incorporated by reference in its entirety). In a specific embodiment, polynucleotides coding for a homodimer of the invention are generated by ligating a polynucleotide sequence encoding a polypeptide of the invention to a sequence encoding a linker polypeptide and then further to a synthetic polynucleotide encoding the translated product of the polypeptide in the reverse orientation from the original C-terminus to the N-terminus (lacking the leader sequence) (see, e.g., U.S. Pat. No. 5,478,925, which is herein incorporated by reference in its entirety). In another embodiment, recombinant techniques described herein or otherwise known in the art are applied to generate recombinant polypeptides of the invention which contain a transmembrane domain (or hyrophobic or signal peptide) and which can be incorporated by membrane reconstitution techniques into liposomes (see, e.g., U.S. Pat. No. 5,478,925, which is herein incorporated by reference in its entirety).

METH1 and METH2 Polypeptides and Fragments

The invention further provides an isolated METH1 polypeptide having the amino acid sequence encoded by the deposited cDNA, or the amino acid sequence in SEQ ID NO:2, or a peptide or polypeptide comprising a portion of the above polypeptides. The invention also provides an isolated METH2 polypeptide having the amino acid sequence encoded by the deposited cDNA, or the amino acid sequence in SEQ ID NO:4, or a peptide or polypeptide comprising a portion of the above polypeptides.

Polypeptides of the present invention encompass not only full length polypeptides, but the mature, proprotein, processed forms of the protein, deletion mutants, substitution variants, allelic variants, analogs, derivatives, etc.

METH1 or METH2 polypeptides can be composed of amino acids joined to each other by peptide bonds or modified peptide bonds, i.e., peptide isosteres, and may contain amino acids other than the 20 gene-encoded amino acids. The METH1 or METH2 polypeptides may be modified by either natural processes, such as post-translational processing, or by chemical modification techniques which are well known in the art. Such modifications are well described in basic texts and in more detailed monographs, as well as in a voluminous research literature. Modifications can occur anywhere in the METH1 or METH2 polypeptide, including the peptide backbone, the amino acid side-chains and the amino or carboxyl termini. It will be appreciated that the same type of modification may be present in the same or varying degrees at several sites in a given METH1 or METH2 polypeptide. Also, a given METH1 or METH2 polypeptide may contain many types of modifications. METH1 or METH2 polypeptides may be branched, for example, as a result of ubiquitination, and they may be cyclic, with or without branching. Cyclic, branched, and branched cyclic METH1 or METH2 polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. (See, for instance, PROTEINS—STRUCTURE AND MOLECULAR PROPERTIES, 2nd Ed., T. E. Creighton, W. H. Freeman and Company, New York (1993); POST-TRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

It will be recognized in the art that some amino acid sequences of the METH1 and METH2 polypeptides can be varied without significant effect of the structure or function of the protein. If such differences in sequence are contemplated, it should be remembered that there will be critical areas on the protein which determine activity.

The present inventors have shown that METH1 and METH2 inhibit angiogenesis in vitro and in vivo. METH1 and METH2 each contain a metalloprotease domain, a disintegrin domain, and TSP-like domains. The metalloprotease domain may be catalytically active. The disintegrin domain may play a role in inhibiting angiogenesis by interacting with integrins, since integrins are essential for the mediation of both proliferative and migratory signals. The present inventors have shown that peptides derived from the TSP-like domains of METH1 and METH2 inhibit angiogenesis in vitro and in vivo.

Thus, the invention further includes variations of the METH1 polypeptide which show substantial METH1 polypeptide activity or which include regions of METH1 protein such as the protein portions discussed below; and variations of the METH2 polypeptide which show substantial METH2 polypeptide activity or which include regions of METH2 protein such as the protein portions discussed below. Such mutants include deletions, insertions, inversions, repeats, and type substitutions. As indicated above, guidance concerning which amino acid changes are likely to be phenotypically silent can be found in Bowie, J. U., et al., “Deciphering the Message in Protein Sequences: Tolerance to Amino Acid Substitutions,” Science 247:1306-1310 (1990).

Thus, the fragment, derivative or analog of the polypeptide of SEQ ID NO:2 or SEQ ID NO:4, or that encoded by the deposited cDNA, may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as an IgG Fc fusion region peptide or leader or secretory sequence or a sequence which is employed for purification of the mature polypeptide or a proprotein sequence. Such fragments, derivatives and analogs are deemed to be within the scope of those skilled in the art from the teachings herein.

Of particular interest are substitutions of charged amino acids with another charged amino acid and with neutral or negatively charged amino acids. The latter results in proteins with reduced positive charge to improve the characteristics of the METH1 or METH2 proteins. The prevention of aggregation is highly desirable. Aggregation of proteins not only results in a loss of activity but can also be problematic when preparing pharmaceutical formulations, because they can be immunogenic. (Pinckard et al., Clin. Exp. Immunol. 2:331-340 (1967); Robbins et al., Diabetes 36:838-845 (1987); Cleland et al., Crit. Rev. Therapeutic Drug Carier Systems 10:307-377 (1993)).

As indicated, changes are preferably of a minor nature, such as conservative amino acid substitutions that do not significantly affect the folding or activity of the protein (see Table 3).

TABLE 3
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

Of course, the number of amino acid substitutions a skilled artisan would make depends on many factors, including those described above. Generally speaking, the number of amino acid substitutions for any given METH1 or METH2 polypeptide will not be more than 50,40, 30, 20, 10, 9, 8,7, 6, 5, 4, 3, 2 or 1.

In particular, preferred METH1 molecules contain one or more of the following conservative substitutions: Ml replaced with A, G, I, L, S, T, or V; G2 replaced with A, I, L, S, T, M, or V; N3 replaced with Q; A4 replaced with G, I, L, S, T, M, or V; E5 replaced with D; R6 replaced with H, or K; A7 replaced with G, I, L, S, T, M, or V; G9 replaced with A, I, L, S, T, M, or V; S10 replaced with A, G, I, L, T, M, or V; R11 replaced with H, or K; S 12 replaced with A, G, I, L, T, M, or V; F13 replaced with W, or Y; G14 replaced with A, I, L, S, T, M, or V; V16 replaced with A, G, I, L, S, T, or M; T18 replaced with A, G, I, L, S, M, or V; L19 replaced with A, G, I, S, T, M, or V; L20 replaced with A, G, I, S, T, M, or V; L21 replaced with A, G, I, S, T, M, or V; L22 replaced with A, G, I, S, T, M, or V; A23 replaced with G, I, L, S, T, M, or V; A24 replaced with G, I, L, S, T, M, or V; A25 replaced with G, I, L, S, T, M, or V; L26 replaced with A, G, I, S, T, M, or V; L27 replaced with A, G, I, S, T, M, or V; A28 replaced with G, I, L, S, T, M, or V; V29 replaced with A, G, I, L, S, T, or M; S30 replaced with A, G, I, L, T, M, or V; D31 replaced with E; A32 replaced with G, I, L, S, T, M, or V; L33 replaced with A, G, I, S, T, M, or V; G34 replaced with A, I, L, S, T, M, or V; R35 replaced with H, or K; S37 replaced with A, G, I, L, T, M, or V; E38 replaced with D; E39 replaced with D; D40 replaced with E; E41 replaced with D; E42 replaced with D; L43 replaced with A, G, I, S, T, M, or V; V44 replaced with A, G, I, L, S, T, or M; V45 replaced with A, G, I, L, S, T, or M; E47 replaced with D; I-48 replaced with A, G, I, S, T, M, or V; E49 replaced with D; R50 replaced with H, or K; A51 replaced with G, I, L, S, T, M, or V; G53 replaced with A, I, L, S, T, M, or V; H54 replaced with K, or R; G55 replaced with A, I, L, S, T, M, or V; T56 replaced with A, G, I, L, S, M, or V; T57 replaced with A, G, I, L, S, M, or V; R58 replaced with H, or K; L59 replaced with A, G, I, S, T, M, or V; R60 replaced with H, or K; L61 replaced with A, G, I, S, T, M, or V; H62 replaced with K, or R; A63 replaced with G, I, L, S, T, M, or V; F64 replaced with W, or Y; D65 replaced with E; Q66 replaced with N; Q67 replaced with N; L68 replaced with A, G, I, S, T, M, or V; D69 replaced with E; L70 replaced with A, G, I, S, T, M, or V; E71 replaced with D; L72 replaced with A, G, I, S, T, M, or V; R73 replaced with H, or K; D75 replaced with E; S76 replaced with A, G, I, L, T, M, or V; S77 replaced with A, G, I, L, T, M, or V; F78 replaced with W, or Y; L79 replaced with A, G, I, S, T, M, or V; A80 replaced with G, I, L, S, T, M, or V; G82 replaced with A, I, L, S, T, M, or V; F83 replaced with W, or Y; T84 replaced with A, G, I, L, S, M, or V; L85 replaced with A, G, I, S, T, M, or V; Q86 replaced with N; N87 replaced with Q; V88 replaced with A, G, I, L, S, T, or M; G89 replaced with A, I, L, S, T, M, or V; R90 replaced with H, or K; K91 replaced with H, or R; S92 replaced with A, G, I, L, T, M, or V; G93 replaced with A, I, L, S, T, M, or V; S94 replaced with A, G, I, L, T, M, or V; E95 replaced with D; T96 replaced with A, G, I, L, S, M, or V; L98 replaced with A, G, I, S, T, M, or V; E100 replaced with D; T101 replaced with A, G, I, L, S, M, or V; D102 replaced with E; L103 replaced with A, G, I, S, T, M, or V; A104 replaced with G, I, L, S, T, M, or V; H105 replaced with K, or R; F107 replaced with W, or Y; Y108 replaced with F, or W; S 109 replaced with A, G, I, L, T, M, or V; G10 replaced with A, I, L, S, T, M, or V; T111 replaced with A, G, I, L, S, M, or V; V112 replaced with A, G, I, L, S, T, or M; N113 replaced with Q; G114 replaced with A, I, L, S, T, M, or V; D115 replaced with E; S117 replaced with A, G, I, L, T, M, or V; S118 replaced with A, G, I, L, T, M, or V; A119 replaced with G, I, L, S, T, M, or V; A120 replaced with G, I, L, S, T, M, or V; A121 replaced with G, I, L, S, T, M, or V; L122 replaced with A, G, I, S, T, M, or V; S 123 replaced with A, G, I, L, T, M, or V; L124 replaced with A, G, I, S, T, M, or V; E126 replaced with D; G127 replaced with A, I, L, S, T, M, or V; V128 replaced with A, G, I, L, S, T, or M; R129 replaced with H, or K; G130 replaced with A, I, L, S, T, M, or V; A131 replaced with G, I, L, S, T, M, or V; F132 replaced with W, or Y; Y133 replaced with F, or W; L134 replaced with A, G, I, S, T, M, or V; L135 replaced with A, G, I, S, T, M, or V; G136 replaced with A, I, L, S, T, M, or V; E137 replaced with D; A138 replaced with G, I, L, S, T, M, or V; Y139 replaced with F, or W; F140 replaced with W, or Y; I141 replaced with A, G, L, S, T, M, or V; Q142 replaced with N; L144 replaced with A, G, I, S, T, M, or V; A146 replaced with G, I, L, S, T, M, or V; A147 replaced with G, I, L, S, T, M, or V; S148 replaced with A, G, I, L, T, M, or V; E149 replaced with D; R150 replaced with H, or K; L151 replaced with A, G, I, S, T, M, or V; A152 replaced with G, I, L, S, T, M, or V; T153 replaced with A, G, I, L, S, M, or V; A154 replaced with G, I, L, S, T, M, or V; A155 replaced with G, I, L, S, T, M, or V; G157 replaced with A, I, L, S, T, M, or V; E158 replaced with D; K159 replaced with H, or R; A162 replaced with G, I, L, S, T, M, or V; L164 replaced with A, G, I, S, T, M, or V; Q165 replaced with N; F166 replaced with W, or Y; H167 replaced with K, or R; L168 replaced with A, G, I, S, T, M, or V; L169 replaced with A, G, I, S, T, M, or V; R170 replaced with H, or K; R171 replaced with H, or K; N172 replaced with Q; R173 replaced with H, or K; Q174 replaced with N; G175 replaced with A, I, L, S, T, M, or V; D176 replaced with E; V177 replaced with A, G, I, L, S, T, or M; G178 replaced with A, I, L, S, T, M, or V; G179 replaced with A, I, L, S, T, M, or V; T180 replaced with A, G, I, L, S, M, or V; G182 replaced with A, I, L, S, T, M, or V; V183 replaced with A, G, I, L, S, T, or M; V184 replaced with A, G, I, L, S, T, or M; D185 replaced with E; D186 replaced with E; E187 replaced with D; R189 replaced with H, or K; T191 replaced with A, G, I, L, S, M, or V; G192 replaced with A, I, L, S, T, M, or V; K193 replaced with H, or R; A194 replaced with G, I, L, S, T, M, or V; E195 replaced with D; T196 replaced with A, G, I, L, S, M, or V; E197 replaced with D; D198 replaced with E; E199 replaced with D; D200 replaced with E; E201 replaced with D; G202 replaced with A, I, L, S, T, M, or V; T203 replaced with A, G, I, L, S, M, or V; E204 replaced with D; G205 replaced with A, I, L, S, T, M, or V; E206 replaced with D; D207 replaced with E; E208 replaced with D; G209 replaced with A, I, L, S, T, M, or V; Q211 replaced with N; W212 replaced with F, or Y; S213 replaced with A, G, I, L, T, M, or V; Q215 replaced with N; D216 replaced with E; A218 replaced with G, I, L, S, T, M, or V; L219 replaced with A, G, I, S, T, M, or V; Q220 replaced with N; G221 replaced with A, I, L, S, T, M, or V; V222 replaced with A, G, I, L, S, T, or M; G223 replaced with A, I, L, S, T, M, or V; Q224 replaced with N; T226 replaced with A, G, I, L, S, M, or V; G227 replaced with A, I, L, S, T, M, or V; T228 replaced with A, G, I, L, S, M, or V; G229 replaced with A, I, L, S, T, M, or V; S230 replaced with A, G, I, L, T, M, or V; I231 replaced with A, G, L, S, T, M, or V; R232 replaced with H, or K; K233 replaced with H, or R; K234 replaced with H, or R; R235 replaced with H, or K; F236 replaced with W, or Y; V237 replaced with A, G, I, L, S, T, or M; S238 replaced with A, G, I, L, T, M, or V; S239 replaced with A, G, I, L, T, M, or V; H240 replaced with K, or R; R241 replaced with H, or K; Y242 replaced with F, or W; V243 replaced with A, G, I, L, S, T, or M; E244 replaced with D; T245 replaced with A, G, I, L, S, M, or V; M246 replaced with A, G, I, L, S, T, or V; L247 replaced with A, G, I, S, T, M, or V; V248 replaced with A, G, I, L, S, T, or M; A249 replaced with G, I, L, S, T, M, or V; D250 replaced with E; Q251 replaced with N; S252 replaced with A, G, I, L, T, M, or V; M253 replaced with A, G, I, L, S, T, or V; A254 replaced with G, I, L, S, T, M, or V; E255 replaced with D; F256 replaced with W, or Y; H257 replaced with K, or R; G258 replaced with A, I, L, S, T, M, or V; S259 replaced with A, G, I, L, T, M, or V; G260 replaced with A, I, L, S, T, M, or V; L261 replaced with A, G, I, S, T, M, or V; K262 replaced with H, or R; H263 replaced with K, or R; Y264 replaced with F, or W; L265 replaced with A, G, I, S, T, M, or V; L266 replaced with A, G, I, S, T, M, or V; T267 replaced with A, G, I, L, S, M, or V; L268 replaced with A, G, I, S, T, M, or V; F269 replaced with W, or Y; S270 replaced with A, G, I, L, T, M, or V; V271 replaced with A, G, I, L, S, T, or M; A272 replaced with G, I, L, S, T, M, or V; A273 replaced with G, I, L, S, T, M, or V; R274 replaced with H, or K; L275 replaced with A, G, I, S, T, M, or V; Y276 replaced with F, or W; K277 replaced with H, or R; H278 replaced with K, or R; S280 replaced with A, G, I, L, T, M, or V; I281 replaced with A, G, L, S, T, M, or V; R282 replaced with H, or K; N283 replaced with Q; S284 replaced with A, G, I, L, T, M, or V; V285 replaced with A, G, I, L, S, T, or M; S286 replaced with A, G, I, L, T, M, or V; L287 replaced with A, G, I, S, T, M, or V; V288 replaced with A, G, I, L, S, T, or M; V289 replaced with A, G, I, L, S, T, or M; V290 replaced with A, G, I, L, S, T, or M; K291 replaced with H, or R; I292 replaced with A, G, L, S, T, M, or V; L293 replaced with A, G, I, S, T, M, or V; V294 replaced with A, G, I, L, S, T, or M; I295 replaced with A, G, L, S, T, M, or V; H296 replaced with K, or R; D297 replaced with E; E298 replaced with D; Q299 replaced with N; K300 replaced with H, or R; G301 replaced with A, I, L, S, T, M, or V; E303 replaced with D; V304 replaced with A, G, I, L, S, T, or M; T305 replaced with A, G, I, L, S, M, or V; S306 replaced with A, G, I, L, T, M, or V; N307 replaced with Q; A308 replaced with G, I, L, S, T, M, or V; A309 replaced with G, I, L, S, T, M, or V; L310 replaced with A, G, I, S, T, M, or V; T311 replaced with A, G, I, L, S, M, or V; L312 replaced with A, G, I, S, T, M, or V; R313 replaced with H, or K; N314 replaced with Q; F315 replaced with W, or Y; N317 replaced with Q; W318 replaced with F, or Y; Q319 replaced with N; K320 replaced with H, or R; Q321 replaced with N; H322 replaced with K, or R; N323 replaced with Q; S326 replaced with A, G, I, L, T, M, or V; D327 replaced with E; R328 replaced with H, or K; D329 replaced with E; A330 replaced with G, I, L, S, T, M, or V; E331 replaced with D; H332 replaced with K, or R; Y333 replaced with F, or W; D334 replaced with E; T335 replaced with A, G, I, L, S, M, or V; A336 replaced with G, I, L, S, T, M, or V; I337 replaced with A, G, L, S, T, M, or V; L338 replaced with A, G, I, S, T, M, or V; F339 replaced with W, or Y; T340 replaced with A, G, I, L, S, M, or V; R341 replaced with H, or K; Q342 replaced with N; D343 replaced with E; L344 replaced with A, G, I, S, T, M, or V; G346 replaced with A, I, L, S, T, M, or V; S347 replaced with A, G, I, L, T, M, or V; Q348 replaced with N; T349 replaced with A, G, I, L, S, M, or V; D351 replaced with E; T352 replaced with A, G, I, L, S, M, or V; L353 replaced with A, G, I, S, T, M, or V; G354 replaced with A, I, L, S, T, M, or V; M355 replaced with A, G, I, L, S, T, or V; A356 replaced with G, I, L, S, T, M, or V; D357 replaced with E; V358 replaced with A, G, I, L, S, T, or M; G359 replaced with A, I, L, S, T, M, or V; T360 replaced with A, G, I, L, S, M, or V; V361 replaced with A, G, I, L, S, T, or M; D363 replaced with E; S365 replaced with A, G, I, L, T, M, or V; R366 replaced with H, or K; S367 replaced with A, G, I, L, T, M, or V; S369 replaced with A, G, I, L, T, M, or V; V370 replaced with A, G, I, L, S, T, or M; I371 replaced with A, G, L, S, T, M, or V; E372 replaced with D; D373 replaced with E; D374 replaced with E; G375 replaced with A, I, L, S, T, M, or V; L376 replaced with A, G, I, S, T, M, or V; Q377 replaced with N; A378 replaced with G, I, L, S, T, M, or V; A379 replaced with G, I, L, S, T, M, or V; F380 replaced with W, or Y; T381 replaced with A, G, I, L, S, M, or V; T382 replaced with A, G, I, L, S, M, or V; A383 replaced with G, I, L, S, T, M, or V; H384 replaced with K, or R; E385 replaced with D; L386 replaced with A, G, I, S, T, M, or V; G387 replaced with A, I, L, S, T, M, or V; H388 replaced with K, or R; V389 replaced with A, G, I, L, S, T, or M; F390 replaced with W, or Y; N391 replaced with Q; M392 replaced with A, G, I, L, S, T, or V; H394 replaced with K, or R; D395 replaced with E; D396 replaced with E; A397 replaced with G, I, L, S, T, M, or V; K398 replaced with H, or R; Q399 replaced with N; A401 replaced with G, I, L, S, T, M, or V; S402 replaced with A, G, I, L, T, M, or V; L403 replaced with A, G, I, S, T, M, or V; N404 replaced with Q; G405 replaced with A, I, L, S, T, M, or V; V406 replaced with A, G, 1, L, S, T, or M; N407 replaced with Q; Q408 replaced with N; D409 replaced with E; S410 replaced with A, G, I, L, T, M, or V; H411 replaced with K, or R; M412 replaced with A, G, I, L, S, T, or V; M413 replaced with A, G, I, L, S, T, or V; A414 replaced with G, I, L, S, T, M, or V; S415 replaced with A, G, I, L, T, M, or V; M416 replaced with A, G, I, L, S, T, or V; I417 replaced with A, G, I, S, T, M, or V; S418 replaced with A, G, I, L, T, M, or V; N419 replaced with Q; I420 replaced with A, G, I, S, T, M, or V; D421 replaced with E; H422 replaced with K, or R; S423 replaced with A, G, I, L, T, M, or V; Q424 replaced with N; W426 replaced with F, or Y; S427 replaced with A, G, I, L, T, M, or V; S430 replaced with A, G, I, L, T, M, or V; A431 replaced with G, I, L, S, T, M, or V; Y432 replaced with F, or W; M433 replaced with A, G, I, L, S, T, or V; I434 replaced with A, G, L, S, T, M, or V; T435 replaced with A, G, I, L, S, M, or V; S436 replaced with A, G, I, L, T, M, or V; F437 replaced with W, or Y; I438 replaced with A, G, I, S, T, M, or V; D439 replaced with E; N440 replaced with Q; G441 replaced with A, I, L, S, T, M, or V; H442 replaced with K, or R; G443 replaced with A, I, L, S, T, M, or V; E444 replaced with D; L446 replaced with A, G, I, S, T, M, or V; M447 replaced with A, G, I, L, S, T, or V; D448 replaced with E; K449 replaced with H, or R; Q451 replaced with N; N452 replaced with Q; I454 replaced with A, G, L, S, T, M, or V; Q455 replaced with N; L456 replaced with A, G, I, S, T, M, or V; G458 replaced with A, I, L, S, T, M, or V; D459 replaced with E; L460 replaced with A, G, I, S, T, M, or V; G462 replaced with A, I, L, S, T, M, or V; T463 replaced with A, G, I, L, S, M, or V; S464 replaced with A, G, I, L, T, M, or V; Y465 replaced with F, or W; D466 replaced with E; A467 replaced with G, I, L, S, T, M, or V; N468 replaced with Q; R469 replaced with H, or K; Q470 replaced with N; Q472 replaced with N; F473 replaced with W, or Y; T474 replaced with A, G, I, L, S, M, or V; F475 replaced with W, or Y; G476 replaced with A, I, L, S, T, M, or V; E477 replaced with D; D478 replaced with E; S479 replaced with A, G, I, L, T, M, or V; K480 replaced with H, or R; H481 replaced with K, or R; D484 replaced with E; A485 replaced with G, I, L, S, T, M, or V; A486 replaced with G, I, L, S, T, M, or V; S487 replaced with A, G, I, L, T, M, or V; T488 replaced with A, G, I, L, S, M, or V; S490 replaced with A, G, I, L, T, M, or V; T491 replaced with A, G, I, L, S, M, or V; L492 replaced with A, G, I, S, T, M, or V; W493 replaced with F, or Y; T495 replaced with A, G, I, L, S, M, or V; G496 replaced with A, I, L, S, T, M, or V; T497 replaced with A, G, I, L, S, M, or V; S498 replaced with A, G, I, L, T, M, or V; G499 replaced with A, I, L, S, T, M, or V; G500 replaced with A, I, L, S, T, M, or V; V501 replaced with A, G, I, L, S, T, or M; L502 replaced with A, I, L, S, T, M, or V; V503 replaced with A, G, I, L, S, T, or M; Q505 replaced with N; T506 replaced with A, G, I, L, S, M, or V; K507 replaced with H, or R; H508 replaced with K, or R; F509 replaced with W, or Y; W511 replaced with F, or Y; A512 replaced with G, I, L, S, T, M, or V; D513 replaced with E; G514 replaced with A, I, L, S, T, M, or V; T515 replaced with A, G, I, L, S, M, or V; S516 replaced with A, G, I, L, T, M, or V; G518 replaced with A, I, L, S, T, M, or V; E519 replaced with D; G520 replaced with A, I, L, S, T, M, or V; K521 replaced with H, or R; W522 replaced with F, or Y; I524 replaced with A, G, L, S, T, M, or V; N525 replaced with Q; G526 replaced with A, I, L, S, T, M, or V; K527 replaced with H, or R; V529 replaced with A, G, I, L, S, T, or M; N530 replaced with Q; K531 replaced with H, or R; T532 replaced with A, G, I, L, S, M, or V; D533 replaced with E; R534 replaced with H, or K; K535 replaced with H, or R; H536 replaced with K, or R; F537 replaced with W, or Y; D538 replaced with E; T539 replaced with A, G, I, L, S, M, or V; F541 replaced with W, or Y; H542 replaced with K, or R; G543 replaced with A, I, L, S, T, M, or V; S544 replaced with A, G, I, L, T, M, or V; W545 replaced with F, or Y; G546 replaced with A, I, L, S, T, M, or V; M547 replaced with A, G, I, L, S, T, or V; W548 replaced with F, or Y; G549 replaced with A, I, L, S, T, M, or V; W551 replaced with F, or Y; G552 replaced with A, I, L, S, T, M, or V; D553 replaced with E; S555 replaced with A, G, I, L, T, M, or V; R556 replaced with H, or K; T557 replaced with A, G, I, L, S, M, or V; G559 replaced with A, I, L, S, T, M, or V; G560 replaced with A, I, L, S, T, M, or V; G561 replaced with A, I, L, S, T, M, or V; V562 replaced with A, G, I, L, S, T, or M; Q563 replaced with N; Y564 replaced with F, or W; T565 replaced with A, G, I, L, S, M, or V; M566 replaced with A, G, I, L, S, T, or V; R567 replaced with H, or K; E568 replaced with D; D570 replaced with E; N571 replaced with Q; V573 replaced with A, G, I, L, S, T, or M; K575 replaced with H, or R; N576 replaced with Q; G577 replaced with A, I, L, S, T, M, or V; G578 replaced with A, I, L, S, T, M, or V; K579 replaced with H, or R; Y580 replaced with F, or W; E582 replaced with D; G583 replaced with A, I, L, S, T, M, or V; K584 replaced with H, or R; R585 replaced with H, or K; V586 replaced with A, G, I, L, S, T, or M; R587 replaced with H, or K; Y588 replaced with F, or W; R589 replaced with H, or K; S590 replaced with A, G, I, L, T, M, or V; N592 replaced with Q; L593 replaced with A, G, I, S, T, M, or V; E594 replaced with D; D595 replaced with E; D598 replaced with E; N599 replaced with Q; N600 replaced with Q; G601 replaced with A, I, L, S, T, M, or V; K602 replaced with H, or R; T603 replaced with A, G, I, L, S, M, or V; F604 replaced with W, or Y; R605 replaced with H, or K; E606 replaced with D; E607 replaced with D; Q608 replaced with N; E610 replaced with D; A611 replaced with G, I, L, S, T, M, or V; H612 replaced with K, or R; N613 replaced with Q; E614 replaced with D; F615 replaced with W, or Y; S616 replaced with A, G, I, L, T, M, or V; K617 replaced with H, or R; A618 replaced with G, I, L, S, T, M, or V; S619 replaced with A, G, I, L, T, M, or V; F620 replaced with W, or Y; G621 replaced with A, I, L, S, T, M, or V; S622 replaced with A, G, I, L, T, M, or V; G623 replaced with A, I, L, S, T, M, or V; A625 replaced with G, I, L, S, T, M, or V; V626 replaced with A, G, I, L, S, T, or M; E627 replaced with D; W628 replaced with F, or Y; I629 replaced with A, G, L, S, T, M, or V; K631 replaced with H, or R; Y632 replaced with F, or W; A633 replaced with G, I, L, S, T, M, or V; G634 replaced with A, I, L, S, T, M, or V; V635 replaced with A, G, I, L, S, T, or M; S636 replaced with A, G, I, L, T, M, or V; K638 replaced with H, or R; D639 replaced with E; R640 replaced with H, or K; K642 replaced with H, or R; L643 replaced with A, G, I, S, T, M, or V; I644 replaced with A, G, L, S, T, M, or V; Q646 replaced with N; A647 replaced with G, I, L, S, T, M, or V; K648 replaced with H, or R; G649 replaced with A, I, L, S, T, M, or V; I650 replaced with A, G, L, S, T, M, or V; G651 replaced with A, I, L, S, T, M, or V; Y652 replaced with F, or W; F653 replaced with W, or Y; F654 replaced with W, or Y; V655 replaced with A, G, I, L, S, T, or M; L656 replaced with A, G, I, S, T, M, or V; Q657 replaced with N; K659 replaced with H, or R; V660 replaced with A, G, I, L, S, T, or M; V661 replaced with A, G, I, L, S, T, or M; D662 replaced with E; G663 replaced with A, I, L, S, T, M, or V; T664 replaced with A, G, I, L, S, M, or V; S667 replaced with A, G, I, L, T, M, or V; D669 replaced with E; S670 replaced with A, G, I, L, T, M, or V; T671 replaced with A, G, I, L, S, M, or V; S672 replaced with A, G, I, L, T, M, or V; V673 replaced with A, G, I, L, S, T, or M; V675 replaced with A, G, I, L, S, T, or M; Q676 replaced with N; G677 replaced with A, I, L, S, T, M, or V; Q678 replaced with N; V680 replaced with A, G, I, L, S, T, or M; K681 replaced with H, or R; A682 replaced with G, I, L, S, T, M, or V; G683 replaced with A, I, L, S, T, M, or V; D685 replaced with E; R686 replaced with H, or K; I687 replaced with A, G, L, S, T, M, or V; I688 replaced with A, G, L, S, T, M, or V; D689 replaced with E; S690 replaced with A, G, I, L, T, M, or V; K691 replaced with H, or R; K692 replaced with H, or R; K693 replaced with H, or R; F694 replaced with W, or Y; D695 replaced with E; K696 replaced with H, or R; G698 replaced with A, I, L, S, T, M, or V; V699 replaced with A, G, I, L, S, T, or M; G701 replaced with A, I, L, S, T, M, or V; G702 replaced with A, I, L, S, T, M, or V; N703 replaced with Q; G704 replaced with A, I, L, S, T, M, or V; S705 replaced with A, G, I, L, T, M, or V; T706 replaced with A, G, I, L, S, M, or V; K708 replaced with H, or R; K709 replaced with H, or R; I710 replaced with A, G, L, S, T, M, or V; S711 replaced with A, G, I, L, T, M, or V; G712 replaced with A, I, L, S, T, M, or V; S713 replaced with A, G, I, L, T, M, or V; V714 replaced with A, G, I, L, S, T, or M; T715 replaced with A, G, I, L, S, M, or V; S716 replaced with A, G, I, L, T, M, or V; A717 replaced with G, I, L, S, T, M, or V; K718 replaced with H, or R; G720 replaced with A, I, L, S, T, M, or V; Y721 replaced with F, or W; H722 replaced with K, or R; D723 replaced with E; I724 replaced with A, G, L, S, T, M, or V; I725 replaced with A, G, L, S, T, M, or V; T726 replaced with A, G, I, L, S, M, or V; I727 replaced with A, G, L, S, T, M, or V; T729 replaced with A, G, I, L, S, M, or V; G730 replaced with A, I, L, S, T, M, or V; A731 replaced with G, I, L, S, T, M, or V; T732 replaced with A, G, I, L, S, M, or V; N733 replaced with Q; I734 replaced with A, G, L, S, T, M, or V; E735 replaced with D; V736 replaced with A, G, I, L, S, T or M; K737 replaced with H, or R; Q738 replaced with N; R739 replaced with H, or K; N740 replaced with Q; Q741 replaced with N; R742 replaced with H, or K; G743 replaced with A, I, L, S, T, M, or V; S744 replaced with A, G, I, L, T, M, or V; R745 replaced with H, or K; N746 replaced with Q; N747 replaced with Q; G748 replaced with A, I, L, S, T, M, or V; S749 replaced with A, G, I, L, T, M, or V; F750 replaced with W, or Y; L751 replaced with A, G, I, S, T, M, or V; A752 replaced with G, I, L, S, T, M, or V; I753 replaced with A, G, L, S, T, M, or V; K754 replaced with H, or R; A755 replaced with G, I, L, S, T, M, or V; A756 replaced with G, I, L, S, T, M, or V; D757 replaced with E; G758 replaced with A, I, L, S, T, M, or V; T759 replaced with A, G, I, L, S, M, or V; Y760 replaced with F, or W; I761 replaced with A, G, L, S, T, M, or V; L762 replaced with A, G, I, S, T, M, or V; N763 replaced with Q; G764 replaced with A, I, L, S, T, M, or V; D765 replaced with E; Y766 replaced with F, or W; T767 replaced with A, G, I, L, S, M, or V; L768 replaced with A, G, I, S, T, M, or V; S769 replaced with A, G, I, L, T, M, or V; T770 replaced with A, G, I, L, S, M, or V; L771 replaced with A, G, I, S, T, M, or V; E772 replaced with D; Q773 replaced with N; D774 replaced with E; I775 replaced with A, G, L, S, T, M, or V; M776 replaced with A, G, I, L, S, T, or V; Y777 replaced with F, or W; K778 replaced with H, or R; G779 replaced with A, I, L, S, T, M, or V; V780 replaced with A, G, I, L, S, T, or M; V781 replaced with A, G, I, L, S, T, or M; L782 replaced with A, G, I, S, T, M, or V; R783 replaced with H, or K; Y784 replaced with F, or W; S785 replaced with A, G, I, L, T, M, or V; G786 replaced with A, I, L, S, T, M, or V; S787 replaced with A, G, I, L, T, M, or V; S788 replaced with A, G, I, L, T, M, or V; A789 replaced with G, I, L, S, T, M, or V; A790 replaced with G, I, L, S, T, M, or V; L791 replaced with A, G, I, S, T, M, or V; E792 replaced with D; R793 replaced with H, or K; I794 replaced with A, G, L, S, T, M, or V; R795 replaced with H, or K; S796 replaced with A, G, I, L, T, M, or V; F797 replaced with W, or Y; S798 replaced with A, G, I, L, T, M, or V; L800 replaced with A, G, I, S, T, M, or V; K801 replaced with H, or R; E802 replaced with D; L804 replaced with A, G, I, S, T, M, or V; T805 replaced with A, G, I, L, S, M, or V; I806 replaced with A, G, L, S, T, M, or V; Q807 replaced with N; V808 replaced with A, G, I, L, S, T, or M; L809 replaced with A, G, I, S, T, M, or V; T810 replaced with A, G, I, L, S, M, or V; V811 replaced with A, G, I, L, S, T, or M; G812 replaced with A, I, L, S, T, M, or V; N813 replaced with Q; A814 replaced with G, I, L, S, T, M, orV; L815 replaced with A, G, I, S, T, M, or V; R816 replaced with H, or K; K818 replaced with H, or R; I819 replaced with A, G, L, S, T, M, or V; K820 replaced with H, or R; Y821 replaced with F, or W; T822 replaced with A, G, I, L, S, M, or V; Y823 replaced with F, or W; F824 replaced with W, or Y; V825 replaced with A, G, I, L, S, T, or M; K826 replaced with H, or R; K827 replaced with H, or R; K828 replaced with H, or R; K829 replaced with H, orR; E830 replaced with D; S831 replaced with A, G, I, L, T, M, orV; F832 replaced with W, or Y; N833 replaced with Q; A834 replaced with G, I, L, S, T, M, or V; I835 replaced with A, G, L, S, T, M, or V; T837 replaced with A, G, I, L, S, M, or V; F838 replaced with W, or Y; S839 replaced with A, G, I, L, T, M, or V; A840 replaced with G, I, L, S, T, M, or V; W841 replaced with F, or Y; V842 replaced with A, G, I, L, S, T, or M; I843 replaced with A, G, L, S, T, M, or V; E844 replaced with D; E845 replaced with D; W846 replaced with F, or Y; G847 replaced with A, I, L, S, T, M, or V; E848 replaced with D; S850 replaced with A, G, I, L, T, M, or V; K851 replaced with H, or R; S852 replaced with A, G, I, L, T, M, or V; E854 replaced with D; L855 replaced with A, G, I, S, T, M, or V; G856 replaced with A, I, L, S, T, M, or V; W857 replaced with F, or Y; Q858 replaced with N; R859 replaced with H, or K; R860 replaced with H, or K; L861 replaced with A, G, I, S, T, M, or V; V862 replaced with A, G, I, L, S, T, or M; E863 replaced with D; R865 replaced with H, or K; D866 replaced with E; I867 replaced with A, G, L, S, T, M, or V; N868 replaced with Q; G869 replaced with A, I, L, S, T, M, or V; Q870 replaced with N; A872 replaced with G, I, L, S, T, M, or V; S873 replaced with A, G, I, L, T, M, or V; E874 replaced with D; A876 replaced with G, I, L, S, T, M, or V; K877 replaced with H, or R; E878 replaced with D; V879 replaced with A, G, I, L, S, T, or M; K880 replaced with H, or R; A882 replaced with G, I, L, S, T, M, or V; S883 replaced with A, G, I, L, T, M, or V; T884 replaced with A, G, I, L, S, M, or V; R885 replaced with H, or K; A888 replaced with G, I, L, S, T, M, or V; D889 replaced with E; H890 replaced with K, or R; Q894 replaced with N; W895 replaced with F, or Y; Q896 replaced with N; L897 replaced with A, G, I, S, T, M, or V; G898 replaced with A, I, L, S, T, M, or V; E899 replaced with D; W900 replaced with F, or Y; S901 replaced with A, G, I, L, T, M, or V; S902 replaced with A, G, I, L, T, M, or V; S904 replaced with A, G, I, L, T, M, or V; K905 replaced with H, or R; T906 replaced with A, G, I, L, S, M, or V; G908 replaced with A, I, L, S, T, M, or V; K909 replaced with H, or R; G910 replaced with A, I, L, S, T, M, or V; Y911 replaced with F, or W; K912 replaced with H, or R; K913 replaced with H, or R; R914 replaced with H, or K; S915 replaced with A, G, I, L, T, M, or V; L916 replaced with A, G, I, S, T, M, or V; K917 replaced with H, or R; L919 replaced with A, G, I, S, T, M, or V; S920 replaced with A, G, I, L, T, M, or V; H921 replaced with K, or R; D922 replaced with E; G923 replaced with A, I, L, S, T, M, or V; G924 replaced with A, I, L, S, T, M, or V; V925 replaced with A, G, I, L, S, T, or M; L926 replaced with A, G, I, S, T, M, or V; S927 replaced with A, G, I, L, T, M, or V; H928 replaced with K, or R; E929 replaced with D; S930 replaced with A, G, I, L, T, M, or V; D932 replaced with E; L934 replaced with A, G, I, S, T, M, or V; K935 replaced with H, or R; K936 replaced with H, or R; K938 replaced with H, or R; H939 replaced with K, or R; F940 replaced with W, or Y; I941 replaced with A, G, L, S, T, M, or V; D942 replaced with E; F943 replaced with W, or Y; T945 replaced with A, G, I, L, S, M, or V; M946 replaced with A, G, I, L, S, T, or V; A947 replaced with G, I, L, S, T, M, or V; E948 replaced with D; S950 replaced with A, G, I, L, T, M, or V.

Also preferred are METH1 polypeptides with one or more of the following non-conservative substitutions: M1 replaced with D, E, H, K, R, N, Q, F, W, Y, P, or C; G2 replaced with D, E, H, K, R, N, Q, F, W, Y, P, or C; N3 replaced with D, E, H, K, R, A, G, I, L, S, T, M, V, F, W, Y, P, or C; A4 replaced with D, E, H, K, R, N, Q, F, W, Y, P, or C; ES replaced with H, K, R, A, G, I, L, S, T, M, V, N, Q, F, W, Y, P, or C; R6 replaced with D, E, A, G, I, L, S, T, M, V, N, Q, F, W, Y, P, or C; A7 replaced with D, E, H, K, R, N, Q, F, W, Y, P, or C; P8 replaced with D, E, H, K, R, A, G, I, L, S, T, M, V, N, Q, F, W, Y, or C; G9 replaced with D, E, H, K, R, N, Q, F, W, Y, P, or C; S1O replaced with D, E, H, K, R, N, Q, F, W, Y, P, or C; R11 replaced with D, E, A, G, I, L, S, T, M, V, N, Q, F, W, Y, P, or C; S12 replaced with D, E, H, K, R, N, Q, F, W, Y, P, or C; F13 replaced with D, E, H, K, R, N, Q, A, G, I, L, S, T, M, V, P, or C; G14 replaced with D, E, H, K, R, N, Q, F, W, Y, P, or C; P15 replaced with D, E, H, K, R, A, G, I, L, S, T, M, V, N, Q, F, W, Y, or C; V16 replaced with D, E, H, K, R, N, Q, F, W, Y, P, or C; P17 replaced with D, E, H, K, R, A, G, I, L, S, T, M, V, N, Q, F, W, Y, or C; T18 replaced with D, E, H, K, R, N, Q, F, W, Y, P, or C; L19 replaced with D, E, H, K, R, N, Q, F, W, Y, P, or C; L20 replaced with D, E, H, K, R, N, Q, F, W, Y, P, or C; L21 replaced with D, E, H, K, R, N, Q, F, W, Y, P, or C; L22 replaced with D, E, H, K, R, N, Q, F, W, Y, P, or C; A23 replaced with D, E, H, K, R, N, Q, F, W, Y, P, or C; A24 replaced with D, E, H, K, R, N, Q, F, W, Y, P, or C; A25 replaced with D, E, H, K, R, N, Q, F, W, Y, P, or C; L26 replaced with D, E, H, K, R, N, Q, F, W, Y, P, or C; L27 replaced with D, E, H, K, R, N, Q, F, W, Y, P, or C; A28 replaced with D, E, H, K, R, N, Q, F, W, Y, P, or C; V29 replaced with D, E, H, K, R, N, Q, F, W, Y, P, or C; S30 replaced with D, E, H, K, R, N, Q, F, W, Y, P, or C; D31 replaced with H, K, R, A, G, I, L, S, T, M, V, N, Q, F, W, Y, P, or C; A32 replaced with D, E, H, K, R, N, Q, F, W, Y, P, or C; L33 replaced with D, E, H, K, R, N, Q, F, W, Y, P, or C; G34 replaced with D, E, H, K, R, N, Q, F, W, Y, P, or C; R35 replaced with D, E, A, G, I, L, S, T, M, V, N, Q, F, W, Y, P, or C; P36replaced with D, E, H, K, R, A, G, I, L, S, T, M, V, N, Q, F, W, Y, or C; S37 replaced with D, E, H, K, R, N, Q, F, W, Y, P, or C; E38 replaced with H, K, R, A, G, I, L, S, T, M, V, N, Q, F, W, Y, P, or C; E39 replaced with H, K, R, A, G, I, L, S, T, M, V, N, Q, F, W, Y, P, or C; D40 replaced with H, K, R, A, G, I, L, S, T, M, V, N, Q, F, W, Y, P, or C; E41 replaced with H, K, R, A, G, I, L, S, T, M, V, N, Q, F, W, Y, P, or C; E42 replaced with H, K, R, A, G, I, L, S, T, M, V, N, Q, F, W, Y, P, or C; L43 replaced with D, E, H, K, R, N, Q, F, W, Y, P, or C; V44 replaced with D, E, H, K, R, N, Q, F, W, Y, P, or C; V45 replaced with D, E, H, K, R, N, Q, F, W, Y, P, or C; P46 replaced with D, E, H, K, R, A, G, I, L, S, T, M, V, N, Q, F, W, Y, or C; E47 replaced with H, K, R, A, G, I, L, S, T, M, V, N, Q, F, W, Y, P, or C; 148 replaced with D, E, H, K, R, N, Q, F, W, Y, P, or C; E49 replaced with H, K, R, A, G, I, L, S, T, M, V, N, Q, F, W, Y, P, or C; R50 replaced with D, E, A, G, I, L, S, T, M, V, N, Q, F, W, Y, P, or C; A51 replaced with D, E, H, K, R, N, Q, F, W, Y, P, or C; P52 replaced with D, E, H, K, R, A, G, I, L, S, T, M, V, N, Q, F, W, Y, or C; G53 replaced with D, E, H, K, R, N, Q, F, W, Y, P, or C; H54 replaced with D, E, A, G, I, L, S, T, M, V, N, Q, F, W, Y, P, or C; G55 replaced with D, E, H, K, R, N, Q, F, W, Y, P, or C; T56 replaced with D, E, H, K, R, N, Q, F, W, Y, P, or C; T57 replaced with D, E, H, K, R, N, Q, F, W, Y, P, or C; R58 replaced with D, E, A, G, I, L, S, T, M, V, N, Q, F, W, Y, P, or C; L59 replaced with D, E, H, K, R, N, Q, F, W, Y, P, or C; R60 replaced with D, E, A, G, I, L, S, T, M, V, N, Q, F, W, Y, P, or C; L61 replaced with D, E, H, K, R, N, Q, F, W, Y, P, or C; H62 replaced with D, E, A, G, I, L, S, T, M, V, N, Q, F, W, Y, P, or C; A63 replaced with D, E, H, K, R, N, Q, F, W, Y, P, or C; F64 replaced with D, E, H, K, R, N, Q, A, G, I, L, S, T, M, V, P, or C; D65 replaced with H, K, R, A, G, I, L, S, T, M, V, N, Q, F, W, Y, P, or C; Q66 replaced with D, E, H, K, R, A, G, I, L, S, T, M, V, F, W, Y, P, or C; Q67 replaced with D, E, H, K, R, A, G, I, L, S, T, M, V, F, W, Y, P, or C; L68 replaced with D, E, H, K, R, N, Q, F, W, Y, P, or C; D69 replaced with H, K, R, A, G, I, L, S, T, M, V, N, Q, F, W, Y, P, or C; L70 replaced with D, E, H, K, R, N, Q, F, W, Y, P, or C; E71 replaced with H, K, R, A, G, I, L, S, T, M, V, N, Q, F, W, Y, P, or C; L72 replaced with D, E, H, K, R, N, Q, F, W, Y, P, or C; R73 replaced with D, E, A, G, I, L, S, T, M, V, N, Q, F, W, Y, P, or C; P74 replaced with D, E, H, K, R, A, G, I, L, S, T, M, V, N, Q, F, W, Y, or C; 1D75 replaced with H, K, R, A, G, I, L, S, T, M, V, N, Q, F, W, Y, P, or C; S76 replaced with D, E, H, K, R, N, Q, F, W, Y, P, or C; S77 replaced with D, E, H, K, R, N, Q, F, W, Y, P, or C; F78 replaced with D, E, H, K, R, N, Q, A, G, I, L, S, T, M, V, P, or C; L79 replaced with D, E, H, K, R, N, Q, F, W, Y, P, or C; A80 replaced with D, E, H, K, R, N, Q, F, W, Y, P, or C; P81 replaced with D, E, H, K, R, A, G, I, L, S, T, M, V, N, Q, F, W, Y, or C; G82 replaced with D, E, H, K, R, N, Q, F, W, Y, P, or C; F83 replaced with D, E, H, K, R, N, Q, A, G, I, L, S, T, M, V, P, or C; T84 replaced with D, E, H, K, R, N, Q, F, W, Y, P, or C; L85 replaced with D, E, H, K, R, N, Q, F, W, Y, P, or C; Q86 replaced with D, E, H, K, R, A, G, I, L, S, T, M, V, F, W, Y, P, or C; N87 replaced with D, E, H, K, R, A, G, I, L, S, T, M, V, F, W, Y, P, or C; V88 replaced with D, E, H, K, R, N, Q, F, W, Y, P, or C; G89 replaced with D, E, H, K, R, N, Q, F, W, Y, P, or C; R90 replaced with D, E, A, G, I, L, S, T, M, V, N, Q, F, W, Y, P, or C; K91 replaced with D, E, A, G, I, L, S, T, M, V, N, Q, F, W, Y, P, or C; S92 replaced with D, E, H, K, R, N, Q, F, W, Y, P, or C; G93 replaced with D, E, H, K, R, N, Q, F, W, Y, P, or C; S94 replaced with D, E, H, K, R, N, Q, F, W, Y, P, or C; E95 replaced with H, K, R, A, G, I, L, S, T, M, V, N, Q, F, W, Y, P, or C; T96 replaced with D, E, H, K, R, N, Q, F, W, Y, P, or C; P97 replaced with D, E, H, K, R, A, G, I, L, S, T, M, V, N, Q, F, W, Y, or C; L98 replaced with D, E, H, K, R, N, Q, F, W, Y, P, or C; P99 replaced with D, L, H, K, R, A, G, I, L, S, T, M, V, N, Q, F, W, Y, or C; E100 replaced with H, K, R, A, G, I, L, S, T, M, V, N, Q, F, W, Y, P, or C; T101 replaced with D, E, H, K, R, N, Q, F, W, Y, P, or C; D102 replaced with H, K, R, A, G, I, L, S, T, M, V, N, Q, F, W, Y, P, or C; L103 replaced with D, E, H, K, R, N, Q, F, W, Y, P, or C; A104 replaced with D, E, H, K, R, N, Q, F, W, Y, P, or C; H105 replaced with D, E, A, G, I, L, S, T, M, V, N, Q, F, W, Y, P, or C; C106 replaced with D, E, H, K, R, A, G, I, L, S, T, M, V, N, Q, F, W, Y, or P; F107 replaced with D, E, H, K, R, N, Q, A, G, I, L, S, T, M, V, P, or C; Y108 replaced with D, E, H, K, R, N, Q, A, G, I, L, S, T, M, V, P, or C; S 109 replaced with D, E, H, K, R, N, Q, F, W, Y, P, or C; G110 replaced with D, E, H, K, R, N, Q, F, W, Y, P, or C; T111 replaced with D, E, H, K, R, N, Q, F, W, Y, P, or C; V112 replaced with D, E, H, K, R, N, Q, F, W, Y, P, or C; N113 replaced with D, E, H, K, R, A, G, I, L, S, T, M, V, F, W, Y, P, or C; G114 replaced with D, E, H, K, R, N, Q, F, W, Y, P, or C; D115 replaced with H, K, R, A, G, I, L, S, T, M, V, N, Q, F, W, Y, P, or C; P116 replaced with D, E, H, K, R, A, G, I, L, S, T, M, V, N, Q, F, W, Y, or C; S117 replaced with D, E, H, K, R, N, Q, F, W, Y, P, or C; S118 replaced with D, E, H, K, R, N, Q, F, W, Y, P, or C; A119 replaced with D, E, H, K, R, N, Q, F, W, Y, P, or C; A120 replaced with D, E, H, K, R, N, Q, F, W, Y, P, or C; A121 replaced with D, E, H, K, R, N, Q, F, W, Y, P, or C; L122 replaced with D, E, H, K, R, N, Q, F, W, Y, P, or C; S123 replaced with D, E, H, K, R, N, Q, F, W, Y, P, or C; L124 replaced with D, E, H, K, R, N, Q, F, W, Y, P, or C; C125 replaced with D, E, H, K, R, A, G, I, L, S, T, M, V, N, Q, F, W, Y, or P; E126 replaced with H, K, R, A, G, I, L, S, T, M, V, N, Q, F, W, Y, P, or C; G127 replaced with D, E, H, K, R, N, Q, F, W, Y, P, or C; V128 replaced with D, E, H, K, R, N, Q, F, W, Y, P, or C; R129 replaced with D, E, A, G, I, L, S, T, M, V, N, Q, F, W, Y, P, or C; G130 replaced with D, E, H, K, R, N, Q, F, W, Y, P, or C; A131 replaced with D, E, H, K, R, N, Q, F, W, Y, P, or C; F132 replaced with D, E, H, K, R, N, Q, A, G, I, L, S, T, M, V, P, or C; Y133 replaced with D, E, H, K, R, N, Q, A, G, I, L, S, T, M, V, P, or C; L134 replaced with D, E, H, K, R, N, Q, F, W, Y, P, or C; L135 replaced with D, E, H, K, R, N, Q, F, W, Y, P, or C; G136 replaced with D, E, H, K, R, N, Q, F, W, Y, P, or C; E137 replaced with H, K, R, A, G, I, L, S, T, M, V, N, Q, F, W, Y, P, or C; A138 replaced with D, E, H, K, R, N, Q, F, W, Y, P, or C; Y139 replaced with D, E, H, K, R, N, Q, A, G, I, L, S, T, M, V, P, or C; F140 replaced with D, E, H, K, R, N, Q, A, G, I, L, S, T, M, V, P, or C; I141 replaced with D, E, H, K, R, N, Q, F, W, Y, P, or C; Q142 replaced with D, E, H, K, R, A, G, I, L, S, T, M, V, F, W, Y, P, or C; P143 replaced with D, E, H, K, R, A, G, I, L, S, T, M, V, N, Q, F, W, Y, or C; L144 replaced with D, E, H, K, R, N, Q, F, W, Y, P, or C; P145 replaced with D, E, H, K, R, A, G, I, L, S, T, M, V, N, Q, F, W, Y, or C; A146 replaced with D, E, H, K, R, N, Q, F, W, Y, P, or C; A147 replaced with D, E, H, K, R, N, Q, F, W, Y, P, or C; S 148 replaced with D, E, H, K, R, N, Q, F, W, Y, P, or C; E149 replaced with H, K, R, A, G, I, L, S, T, M, V, N, Q, F, W, Y, P, or C; R150 replaced with D, E, A, G, I, L, S, T, M, V, N, Q, F, W, Y, P, or C; L151 replaced with D, E, H, K, R, N, Q, F, W, Y, P, or C; A152 replaced with D, E, H, K, R, N, Q, F, W, Y, P, or C; T153 replaced with D, E, H, K, R, N, Q, F, W, Y, P, or C; A154 replaced with D, E, H, K, R, N, Q, F, W, Y, P, or C; A155replaced with D, E, H, K, R, N, Q, F, W, Y, P, or C; P156 replaced with D, E, H, K, R, A, G, , L, S, T, M, V, N, Q, F, W, Y, or C; G157 replaced with D, E, H, K, R, N, Q, F, W, Y, P, or C; E158 replaced with H, K, R, A, G, I, L, S, T, M, V, N, Q, F, W, Y, P, or C; K159 replaced with D, E, A, G, I, L, S, T, M, V, N, Q, F, W, Y, P, or C; P160 replaced with D, E, H, K, R, A, G, I, L, S, T, M, V, N, Q, F, W, Y, or C; P161 replaced with D, E, H, K, R, A, G, I, L, S, T, M, V, N, Q, F, W, Y, or C; A162 replaced with D, E, H, K, R, N, Q, F, W, Y, P, or C; P163 replaced with D, E, H, K, R, A, G, I, L, S, T, M, V, N, Q, F, W, Y, or C; L164 replaced with D, E, H, K, R, N, Q, F, W, Y, P, or C; Q165 replaced with D, E, H, K, R, A, G, I, L, S, T, M, V, F, W, Y, P, or C; F166 replaced with D, E, H, K, R, N, Q, A, G, I, L, S, T, M, V, P, or C; H167 replaced with D, E, A, G, I, L, S, T, M, V, N, Q, F, W, Y, P, or C; L168 replaced with D, E, H, K, R, N, Q, F, W, Y, P, or C; L169 replaced with D, E, H, K, R, N, Q, F, W, Y, P, or C; R170 replaced with D, E, A, G, I, L, S, T, M, V, N, Q, F, W, Y, P, or C; R171 replaced with D, E, A, G, I, L, S, T, M, V, N, Q, F, W, Y, P, or C; N172 replaced with D, E, H, K, R, A, G, I, L, S, T, M, V, F, W, Y, P, or C; R173 replaced with D, E, A, G, I, L, S, T, M, V, N, Q, F, W, Y, P, or C; Q174 replaced with D, E, H, K, R, A, G, I, L, S, T, M, V, F, W, Y, P, or C; G175 replaced with D, E, H, K, R, N, Q, F, W, Y, P, or C; D176 replaced with H, K, R, A, G, I, L, S, T, M, V, N, Q, F, W, Y, P, or C; V177 replaced with D, E, H, K, R, N, Q, F, W, Y, P, or C; G178 replaced with D, E, H, K, R, N, Q, F, W, Y, P, or C; G179 replaced with D, E, H, K, R, N, Q, F, W, Y, P, or C; T180 replaced with D, E, H, K, R, N, Q, F, W, Y, P, or C; C181 replaced with D, E, H, K, R, A, G, I, L, S, T, M, V, N, Q, F, W, Y, or P; G182 replaced with D, E, H, K, R, N, Q, F, W, Y, P, or C; V183 replaced with D, E, H, K, R, N, Q, F, W, Y, P, or C; V184 replaced with D, E, H, K, R, N, Q, F, W, Y, P, or C; D185 replaced with H, K, R, A, G, I, L, S, T, M, V, N, Q, F, W, Y, P, or C; D186 replaced with H, K, R, A, G, I, L, S, T, M, V, N, Q, F, W, Y, P, or C; E187 replaced with H, K, R, A, G, I, L, S, T, M, V, N, Q, F, W, Y, P, or C; P188 replaced with D, E, H, K, R, A, G, I, L, S, T, M, V, N, Q, F, W, Y, or C; R189 replaced with D, E, A, G, I, L, S, T, M, V, N, Q, F, W, Y, P, or C; P190 replaced with D, E, H, K, R, A, G, I, L, S, T, M, V, N, Q, F, W, Y, or C; T191 replaced with D, E, H, K, R, N, Q, F, W, Y, P, or C; G192 replaced with D, E, H, K, R, N, Q, F, W, Y, P, or C; K193 replaced with D, E, A, G, I, L, S, T, M, V, N, Q, F, W, Y, P, or C; A194 replaced with D, E, H, K, R, N, Q, F, W, Y, P, or C; E195 replaced with H, K, R, A, G, I, L, S, T, M, V, N, Q, F, W, Y, P, or C; T196 replaced with D, E, H, K, R, N, Q, F, W, Y, P, or C; E197 replaced with H, K, R, A, G, I, L, S, T, M, V, N, Q, F, W, Y, P, or C; D198 replaced with H, K, R, A, G, I, L, S, T, M, V, N, Q, F, W, Y, P, or C; E199 replaced with H, K, R, A, G, I, L, S, T, M, V, N, Q, F, W, Y, P, or C; D200 replaced with H, K, R, A, G, I, L, S, T, M, V, N, Q, F, W, Y, P, or C; E201 replaced with H, K, R, A, G, I, L, S, T, M, V, N, Q, F, W, Y, P, or C; G202 replaced with D, E, H, K, R, N, Q, F, W, Y, P, or C; T203 replaced with D, E, H, K, R, N, Q, F, W, Y, P, or C; E204 replaced with H, K, R, A, G, I, L, S, T, M, V, N, Q, F, W, Y, P, or C; G205 replaced with D, E, H, K, R, N, Q, F, W, Y, P, or C; E206 replaced with H, K, R, A, G, I, L, S, T, M, V, N, Q, F, W, Y, P, or C; D207 replaced with H, K, R, A, G, I, L, S, T, M, V, N, Q, F, W, Y, P, or C; E208 replaced with H, K, R, A, G, I, L, S, T, M, V, N, Q, F, W, Y, P, or C; G209 replaced with D, E, H, K, R, N, Q, F, W, Y, P, or C; P210 replaced with D, E, H, K, R, A, G, I, L, S, T, M, V, N, Q, F, W, Y, or C; Q211 replaced with D, E, H, K, R, A, G, I, L, S, T, M, V, F, W, Y, P, or C; W212 replaced with D, E, H, K, R, N, Q, A, G, I, L, S, T, M, V, P, or C; S213 replaced with D, E, H, K, R, N, Q, F, W, Y, P, or C; P214 replaced with D, E, H, K, R, A, G, I, L, S, T, M, V, N, Q, F, W, Y, or C; Q215 replaced with D, E, H, K, R, A, G, I, L, S, T, M, V, F, W, Y, P, or C; D216 replaced with H, K, R, A, G, I, L, S, T, M, V, N, Q, F, W, Y, P, or C; P217 replaced with D, E, H, K, R, A, G, I, L, S, T, M, V, N, Q, F, W, Y, or C; A218 replaced with D, E, H, K, R, N, Q, F, W, Y, P, or C; L219 replaced with D, E, H, K, R, N, Q, F, W, Y, P, or C; Q220 replaced with D, E, H, K, R, A, G, I, L, S, T, M, V, F, W, Y, P, or C; G221 replaced with D, E, H, K, R, N, Q, F, W, Y, P, or C; V222 replaced with D, E, H, K, R, N, Q, F, W, Y, P, or C; G223 replaced with D, E, H, K, R, N, Q, F, W, Y, P, or C; Q224 replaced with D, E, H, K, R, A, G, I, L, S, T, M, V, F, W, Y, P, or C; P225 replaced with D, E, H, K, R, A, G, I, L, S, T, M, V, N, Q, F, W, Y, or C; T226 replaced with D, E, H, K, R, N, Q, F, W, Y, P, or C; G227 replaced with D, E, H, K, R, N, Q, F, W, Y, P, or C; T228 replaced with D, E, H, K, R, N, Q, F, W, Y, P, or C; G229 replaced with D, E, H, K, R, N, Q, F, W, Y, P, or C; S230 replaced with D, E, H, K, R, N, Q, F, W, Y, P, or C; I231 replaced with D, E, H, K, R, N, Q, F, W, Y, P, or C; R232 replaced with D, E, A, G, I, L, S, T, M, V, N, Q, F, W, Y, P, or C; K233 replaced with D, E, A, G, I, L, S, T, M, V, N, Q, F, W, Y, P, or C; K234 replaced with D, E, A, G, I, L, S, T, M, V, N, Q, F, W, Y, P, or C; R235 replaced with D, E, A, G, I, L, S, T, M, V, N, Q, F, W, Y, P, or C; F236 replaced with D, E, H, K, R, N, Q, A, G, I, L, S, T, M, V, P, or C; V237 replaced with D, E, H, K, R, N, Q, F, W, Y, P, or C; S238 replaced with D, E, H, K, R, N, Q, F, W, Y, P, or C; S239 replaced with D, E, H, K, R, N, Q, F, W, Y, P, or C; H240 replaced with D, E, A, G, I, L, S, T, M, V, N, Q, F, W, Y, P, or C; R241 replaced with D, E, A, G, I, L, S, T, M, V, N, Q, F, W, Y, P, or C; Y242 replaced with D, E, H, K, R, N, Q, A, G, I, L, S, T, M, V, P, or C; V243 replaced with D, E, H, K, R, N, Q, F, W, Y, P, or C; E244 replaced with H, K, R, A, G, I, L, S, T, M, V, N, Q, F, W, Y, P, or C; T245 replaced with D, E, H, K, R, N, Q, F, W, Y, P, or C; M246 replaced with D, E, H, K, R, N, Q, F, W, Y, P, or C; L247 replaced with D, E, H, K, R, N, Q, F, W, Y, P, or C; V248 replaced with D, E, H, K, R, N, Q, F, W, Y, P, or C; A249 replaced with D, E, H, K, R, N, Q, F, W, Y, P, or C; D250 replaced with H, K, R, A, G, I, L, S, T, M, V, N, Q, F, W, Y, P, or C; Q251 replaced with D, E, H, K, R, A, G, I, L, S, T, M, V, F, W, Y, P, or C; S252 replaced with D, E, H, K, R, N, Q, F, W, Y, P, or C; M253 replaced with D, E, H, K, R, N, Q, F, W, Y, P, or C; A254 replaced with D, E, H, K, R, N, Q, F, W, Y, P, or C; E255 replaced with H, K, R, A, G, I, L, S, T, M, V, N, Q, F, W, Y, P, or C; F256 replaced with D, E, H, K, R, N, Q, A, G, I, L, S, T, M, V, P, or C; H257replaced with D, E, A, G, I, L, S, T, M, V, N, Q, F, W, Y, P, or C; G258 replaced with D, E, H, K, R, N, Q, F, W, Y, P, or C; S259 replaced with D, E, H, K, R, N, Q, F, W, Y, P, or C; G260 replaced with D, E, H, K, R, N, Q, F, W, Y, P, or C; L261 replaced with D, E, H, K, R, N, Q, F, W, Y, P, or C; K262 replaced with D, E, A, G, I, L, S, T, M, V, N, Q, F, W, Y, P, or C; H263 replaced with D, E, A, G, I, L, S, T, M, V, N, Q, F, W, Y, P, or C; Y264 replaced with D, E, H, K, R, N, Q, A, G, I, L, S, T, M, V, P, or C; L265 replaced with D, E, H, K, R, N, Q, F, W, Y, P, or C; L266 replaced with D, E, H, K, R, N, Q, F, W, Y, P, or C; T267 replaced with D, E, H, K, R, N, Q, F, W, Y, P, or C; L268 replaced with D, E, H, K, R, N, Q, F, W, Y, P, or C; F269 replaced with D, E, H, K, R, N, Q, A, G, I, L, S, T, M, V, P, or C; S270 replaced with D, E, H, K, R, N, Q, F, W, Y, P, or C; V271 replaced with D, E, H, K, R, N, Q, F, W, Y, P, or C; A272 replaced with D, E, H, K, R, N, Q, F, W, Y, P, or C; A273 replaced with D, E, H, K, R, N, Q, F, W, Y, P, or C; R274 replaced with D, E, A, G, I, L, S, T, M, V, N, Q, F, W, Y, P, or C; L275 replaced with D, E, H, K, R, N, Q, F, W, Y, P, or C; Y276 replaced with D, E, H, K, R, N, Q, A, G, I, L, S, T, M, V, P, or C; K277 replaced with D, E, A, G, I, L, S, T, M, V, N, Q, F, W, Y, P, or C; H278 replaced with D, E, A, G, I, L, S, T, M, V, N, Q, F, W, Y, P, or C; P279 replaced with D, E, H, K, R, A, G, I, L, S, T, M, V, N, Q, F, W, Y, or C; S280 replaced with D, E, H, K, R, N, Q, F, W, Y, P, or C; I281 replaced with D, E, H, K, R, N, Q, F, W, Y, P, or C; R282 replaced with D, E, A, G, I, L, S, T, M, V, N, Q, F, W, Y, P, or C; N283 replaced with D, E, H, K, R, A, G, I, L, S, T, M, V, F, W, Y, P, or C; S284 replaced with D, E, H, K, R, N, Q, F, W, Y, P, or C; V285 replaced with D, E, H, K, R, N, Q, F, W, Y, P, or C; S286 replaced with D, E, H, K, R, N, Q, F, W, Y, P, or C; L287 replaced with D, E, H, K, R, N, Q, F, W, Y, P, or C; V288 replaced with D, E, H, K, R, N, Q, F, W, Y, P, or C; V289 replaced with D, E, H, K, R, N, Q, F, W, Y, P, or C; V290 replaced with D, E, H, K, R, N, Q, F, W, Y, P, or C; K291 replaced with D, E, A, G, I, L, S, T, M, V, N, Q, F, W, Y, P, or C; I292 replaced with D, E, H, K, R, N, Q, F, W, Y, P, or C; L293 replaced with D, E, H, K, R, N, Q, F, W, Y, P, or C; V294 replaced with D, E, H, K, R, N, Q, F, W, Y, P, or C; I295 replaced with D, E, H, K, R, N, Q, F, W, Y, P, or C; H296 replaced with D, E, A, G, I, L, S, T, M, V, N, Q, F, W, Y, P, or C; D297 replaced with H, K, R, A, G, I, L, S, T, M, V, N, Q, F, W, Y, P, or C; E298 replaced with H, K, R, A, G, I, L, S, T, M, V, N, Q, F, W, Y, P, or C; Q299 replaced with D, E, H, K, R, A, G, I, L, S, T, M, V, F, W, Y, P, or C; K300 replaced with D, E, A, G, I, L, S, T, M, V, N, Q, F, W, Y, P, or C; G301 replaced with D, E, H, K, R, N, Q, F, W, Y, P, or C; P302 replaced with D, E, H, K, R, A, G, I, L, S, T, M, V, N, Q, F, W, Y, or C; E303 replaced with H, K, R, A, G, I, L, S, T, M, V, N, Q, F, W, Y, P, or C; V304 replaced with D, E, H, K, R, N, Q, F, W, Y, P, or C; T305 replaced with D, E, H, K, R, N, Q, F, W, Y, P, or C; S306 replaced with D, E, H, K, R, N, Q, F, W, Y, P, or C; N307 replaced with D, E, H, K, R, A, G, I, L, S, T, M, V, F, W, Y, P, or C; A308 replaced with D, E, H, K, R, N, Q, F, W, Y, P, or C; A309 replaced with D, E, H, K, R, N, Q, F, W, Y, P, or C; L310 replaced with D, E, H, K, R, N, Q, F, W, Y, P, or C; T311 replaced with D, E, H, K, R, N, Q, F, W, Y, P, or C; L312 replaced with D, E, H, K, R, N, Q, F, W, Y, P, or C; R313 replaced with D, E, A, G, I, L, S, T, M, V, N, Q, F, W, Y, P, or C; N314 replaced with D, E, H, K, R, A, G, I, L, S, T, M, V, F, W, Y, P, or C; F315 replaced with D, E, H, K, R, N, Q, A, G, I, L, S, T, M, V, P, or C; C316 replaced with D, E, H, K, R, A, G, I, L, S, T, M, V, N, Q, F, W, Y, or P; N317 replaced with D, E, H, K, R, A, G, I, L, S, T, M, V, F, W, Y, P, or C; W318 replaced with D, E, H, K, R, N, Q, A, G, I, L, S, T, M, V, P, or C; Q319 replaced with D, E, H, K, R, A, G, I, L, S, T, M, V, F, W, Y, P, or C; K320 replaced with D, E, A, G, I, L, S, T, M, V, N, Q, F, W, Y, P, or C; Q321 replaced with D, E, H, K, R, A, G, I, L, S, T, M, V, F, W, Y, P, or C; H322 replaced with D, E, A, G, I, L, S, T, M, V, N, Q, F, W, Y, P, or C; N323 replaced with D, E, H, K, R, A, G, I, L, S, T, M, V, F, W, Y, P, or C; P324 replaced with D, E, H, K, R, A, G, I, L, S, T, M, V, N, Q, F, W, Y, or C; P325 replaced with D, E, H, K, R, A, G, I, L, S, T, M, V, N, Q, F, W, Y, or C; S326 replaced with D, E, H, K, R, N, Q, F, W, Y, P, or C; D327 replaced with H, K, R, A, G, I, L, S, T, M, V, N, Q, F, W, Y, P, or C; R328 replaced with D, E, A, G, I, L, S, T, M, V, N, Q, F, W, Y, P, or C; D329 replaced with H, K, R, A, G, I, L, S, T, M, V, N, Q, F, W, Y, P, or C; A330 replaced with D, E, H, K, R, N, Q, F, W, Y, P, or C; E331 replaced with H, K, R, A, G, I, L, S, T, M, V, N, Q, F, W, Y, P, or C; H332 replaced with D, E, A, G, I, L, S, T, M, V, N, Q, F, W, Y, P, or C; Y333 replaced with D, E, H, K, R, N, Q, A, G, I, L, S, T, M, V, P, or C; D334 replaced with H, K, R, A, G, I, L, S, T, M, V, N, Q, F, W, Y, P, or C; T335 replaced with D, E, H, K, R, N, Q, F, W, Y, P, or C; A336 replaced with D, E, H, K, R, N, Q, F, W, Y, P, or C; I337 replaced with D, E, H, K, R, N, Q, F, W, Y, P, or C; L338 replaced with D, E, H, K, R, N, Q, F, W, Y, P, or C; F339 replaced with D, E, H, K, R, N, Q, A, G, I, L, S, T, M, V, P, or C; T340 replaced with D, E, H, K, R, N, Q, F, W, Y, P, or C; R341 replaced with D, E, A, G, I, L, S, T, M, V, N, Q, F, W, Y, P, or C; Q342 replaced with D, E, H, K, R, A, G, I, L, S, T, M, V, F, W, Y, P, or C; D343 replaced with H, K, R, A, G, I, L, S, T, M, V, N, Q, F, W, Y, P, or C; L344 replaced with D, E, H, K, R, N, Q, F, W, Y, P, or C; C345 replaced with D, E, H, K, R, A, G, I, L, S, T, M, V, N, Q, F, W, Y, or P; G346 replaced with D, E, H, K, R, N, Q, F, W, Y, P, or C; S347 replaced with D, E, H, K, R, N, Q, F, W, Y, P, or C; Q348 replaced with D, E, H, K, R, A, G, I, L, S, T, M, V, F, W, Y, P, or C; T349 replaced with D, E, H, K, R, N, Q, F, W, Y, P, or C; C350 replaced with D, E, H, K, R, A, G, I, L, S, T, M, V, N, Q, F, W, Y, or P; D351 replaced with H, K, R, A, G, I, L, S, T, M, V, N, Q, F, W, Y, P, or C; T352 replaced with D, E, H, K, R, N, Q, F, W, Y, P, or C; L353 replaced with D, E, H, K, R, N, Q, F, W, Y, P, or C; G354 replaced with D, E, H, K, R, N, Q, F, W, Y, P, or C; M355 replaced with D, E, H, K, R, N, Q, F, W, Y, P, or C; A356 replaced with D, E, H, K, R, N, Q, F, W, Y, P, or C; D357 replaced with H, K, R, A, G, I, L, S, T, M, V, N, Q, F, W, Y, P, or C; V358 replaced with D, E, H, K, R, N, Q, F, W, Y, P, or C; G359 replaced with D, E, H, K, R, N, Q, F, W, Y, P, or C; T360 replaced with D, E, H, K, R, N, Q, F, W, Y, P, or C; V361 replaced with D, E, H, K, R, N, Q, F, W, Y, P, or C; C362 replaced with D, E, H, K, R, A, G, I, L, S, T, M, V, N, Q, F, W, Y, or P; D363 replaced with H, K, R, A, G, I, L, S, T, M, V, N, Q, F, W, Y, P, or C; P364 replaced with D, E, H, K, R, A, G, I, L, S, T, M, V, N, Q, F, W, Y, or C; S365 replaced with D, E, H, K, R, N, Q, F, W, Y, P, or C; R366 replaced with D, E, A, G, I, L, S, T, M, V, N, Q, F, W, Y, P, or C; S367 replaced with D, E, H, K, R, N, Q, F, W, Y, P, or C; C368 replaced with D, E, H, K, R, A, G, I, L, S, T, M, V, N, Q, F, W, Y, or P; S369 replaced with D, E, H, K, R, N, Q, F, W, Y, P, or C; V370 replaced with D, E, H, K, R, N, Q, F, W, Y, P, or C; I371 replaced with D, E, H, K, R, N, Q, F, W, Y, P, or C; E372 replaced with H, K, R, A, G, I, L, S, T, M, V, N, Q, F, W, Y, P, or C; D373 replaced with H, K, R, A, G, I, L, S, T, M, V, N, Q, F, W, Y, P, or C; D374 replaced with H, K, R, A, G, I, L, S, T, M, V, N, Q, F, W, Y, P, or C; G375 replaced with D, E, H, K, R, N, Q, F, W, Y, P, or C; L376 replaced with D, E, H, K, R, N, Q, F, W, Y, P, or C; Q377 replaced with D, E, H, K, R, A, G, I, L, S, T, M, V, F, W, Y, P, or C; A378 replaced with D, E, H, K, R, N, Q, F, W, Y, P, or C; A379 replaced with D, E, H, K, R, N, Q, F, W, Y, P, or C; F380 replaced with D, E, H, K, R, N, Q, A, G, I, L, S, T, M, V, P, or C; T381 replaced with D, E, H, K, R, N, Q, F, W, Y, P, or C; T382 replaced with D, E, H, K, R, N, Q, F, W, Y, P, or C; A383 replaced with D, E, H, K, R, N, Q, F, W, Y, P, or C; H384 replaced with D, E, A, G, I, L, S, T, M, V, N, Q, F, W, Y, P, or C; E385 replaced with H, K, R, A, G, I, L, S, T, M, V, N, Q, F, W, Y, P, or C; L386 replaced with D, E, H, K, R, N, Q, F, W, Y, P, or C; G387 replaced with D, E, H, K, R, N, Q, F, W, Y, P, or C; H388 replaced with D, E, A, G, I, L, S, T, M, V, N, Q, F, W, Y, P, or C; V389 replaced with D, E, H, K, R, N, Q, F, W, Y, P, or C; F390 replaced with D, E, H, K, R, N, Q, A, G, I, L, S, T, M, V, P, or C; N391 replaced with D, E, H, K, R, A, G, I, L, S, T, M, V, F, W, Y, P, or C; M392 replaced with D, E, H, K, R, N, Q, F, W, Y, P, or C; P393 replaced with D, E, H, K, R, A, G, I, L, S, T, M, V, N, Q, F, W, Y, or C; H394 replaced with D, E, A, G, I, L, S, T, M, V, N, Q, F, W, Y, P, or C; D395 replaced with H, K, R, A, G, I, L, S, T, M, V, N, Q, F, W, Y, P, or C; D396 replaced with H, K, R, A, G, I, L, S, T, M, V, N, Q, F, W, Y, P, or C; A397 replaced with D, E, H, K, R, N, Q, F, W, Y, P, or C; K398 replaced with D, E, A, G, I, L, S, T, M, V, N, Q, F, W, Y, P, or C; Q399 replaced with D, E, H, K, R, A, G, I, L, S, T, M, V, F, W, Y, P, or C; C400 replaced with D, E, H, K, R, A, G, I, L, S, T, M, V, N, Q, F, W, Y, or P; A401 replaced with D, E, H, K, R, N, Q, F, W, Y, P, or C; S402 replaced with D, E, H, K, R, N, Q, F, W, Y, P, or C; L403 replaced with D, E, H, K, R, N, Q, F, W, Y, P, or C; N404 replaced with D, E, H, K, R, A, G, I, L, S, T, M, V, F, W, Y, P, or C; G405 replaced with D, E, H, K, R, N, Q, F, W, Y, P, or C; V406 replaced with D, E, H, K, R, N, Q, F, W, Y, P, or C; N407 replaced with D, E, H, K, R, A, G, I, L, S, T, M, V, F, W, Y, P, or C; Q408 replaced with D, E, H, K, R, A, G, I, L, S, T, M, V, F, W, Y, P, or C; D409 replaced with H, K, R, A, G, I, L, S, T, M, V, N, Q, F, W, Y, P, or C; S410 replaced with D, E, H, K, R, N, Q, F, W, Y, P, or C; H411 replaced with D, E, A, G, I, L, S, T, M, V, N, Q, F, W, Y, P, or C; M412 replaced with D, E, H, K, R, N, Q, F, W, Y, P, or C; M413 replaced with D, E, H, K, R, N, Q, F, W, Y, P, or C; A414 replaced with D, E, H, K, R, N, Q, F, W, Y, P, or C; S415 replaced with D, E, H, K, R, N, Q, F, W, Y, P, or C; M416 replaced with D, E, H, K, R, N, Q, F, W, Y, P, or C; I417 replaced with D, E, H, K, R, N, Q, F, W, Y, P, or C; S418 replaced with D, E, H, K, R, N, Q, F, W, Y, P, or C; N419 replaced with D, E, H, K, R, A, G, I, L, S, T, M, V, F, W, Y, P, or C; L420 replaced with D, E, H, K, R, N, Q, F, W, Y, P, or C; D421 replaced with H, K, R, A, G, I, L, S, T, M, V, N, Q, F, W, Y, P, or C; H422 replaced with D, E, A, G, I, L, S, T, M, V, N, Q, F, W, Y, P, or C; S423 replaced with D, E, H, K, R, N, Q, F, W, Y, P, or C; Q424 replaced with D, E, H, K, R, A, G, I, L, S, T, M, V, F, W, Y, P, or C; P425 replaced with D, E, H, K, R, A, G, I, L, S, T, M, V, N, Q, F, W, Y, or C; W426 replaced with D, E, H, K, R, N, Q, A, G, I, L, S, T, M, V, P, or C; S427 replaced with D, E, H, K, R, N, Q, F, W, Y, P, or C; P428 replaced with D, E, H, K, R, A, G, I, L, S, T, M, V, N, Q, F, W, Y, or C; C429 replaced with D, E, H, K, R, A, G, I, L, S, T, M, V, N, Q, F, W, Y, or P; S430 replaced with D, E, H, K, R, N, Q, F, W, Y, P, or C; A431 replaced with D, E, H, K, R, N, Q, F, W, Y, P, or C; Y432 replaced with D, E, H, K, R, N, Q, A, G, I, L, S, T, M, V, P, or C; M433 replaced with D, E, H, K, R, N, Q, F, W, Y, P, or C; I434 replaced with D, E, H, K, R, N, Q, F, W, Y, P, or C; T435 replaced with D, E, H, K, R, N, Q, F, W, Y, P, or C; S436 replaced with D, E, H, K, R, N, Q, F, W, Y, P, or C; F437 replaced with D, E, H, K, R, N, Q, A, G, I, L, S, T, M, V, P, or C; L438 replaced with D, E, H, K, R, N, Q, F, W, Y, P, or C; D439 replaced with H, K, R, A, G, I, L, S, T, M, V, N, Q, F, W, Y, P, or C; N440replaced with D, E, H, K, R, A, G, I, L, S, T, M, V, F, W, Y, P, or C; G441 replaced with D, E, H, K, R, N, Q, F, W, Y, P, or C; H442 replaced with D, E, A, G, I, L, S, T, M, V, N, Q, F, W, Y, P, or C; G443 replaced with D, E, H, K, R, N, Q, F, W, Y, P, or C; E444 replaced with H, K, R, A, G, I, L, S, T, M, V, N, Q, F, W, Y, P, or C; C445 replaced with D, E, H, K, R, A, G, I, L, S, T, M, V, N, Q, F, W, Y, or P; L446 replaced with D, E, H, K, R, N, Q, F, W, Y, P, or C; M447 replaced with D, E, H, K, R, N, Q, F, W, Y, P, or C; D448 replaced with H, K, R, A, G, I, L, S, T, M, V, N, Q, F, W, Y, P, or C; K449 replaced with D, E, A, G, I, L, S, T, M, V, N, Q, F, W, Y, P, or C; P450 replaced with D, E, H, K, R, A, G, I, L, S, T, M, V, N, Q, F, W, Y, or C; Q451 replaced with D, E, H, K, R, A, G, I, L, S, T, M, V, F, W, Y, P, or C; N452 replaced with D, E, H, K, R, A, G, I, L, S, T, M, V, F, W, Y, P, or C; P453 replaced with D, E, H, K, R, A, G, I, L, S, T, M, V, N, Q, F, W, Y, or C; I454 replaced with D, E, H, K, R, N, Q, F, W, Y, P, or C; Q455 replaced with D, E, H, K, R, A, G, I, L, S, T, M, V, F, W, Y, P, or C; I456 replaced with D, E, H, K, R, N, Q, F, W, Y, P, or C; P457 replaced with D, E, H, K, R, A, G, I, L, S, T, M, V, N, Q, F, W, Y, or C; G458 replaced with D, E, H, K, R, N, Q, F, W, Y, P, or C; D459 replaced with H, K, R, A, G, I, L, S, T, M, V, N, Q, F, W, Y, P, or C; L460 replaced with D, E, H, K, R, N, Q, F, W, Y, P, or C; P461 replaced with D, E, H, K, R, A, G, I, L, S, T, M, V, N, Q, F, W, Y, or C; G462 replaced with D, E, H, K, R, N, Q, F, W, Y, P, or C; T463 replaced with D, E, H, K, R, N, Q, F, W, Y, P, or C; S464 replaced with D, E, H, K, R, N, Q, F, W, Y, P, or C; Y465 replaced with D, E, H, K, R, N, Q, A, G, I, L, S, T, M, V, P, or C; D466 replaced with H, K, R, A, G, I, L, S, T, M, V, N, Q, F, W, Y, P, or C; A467 replaced with D, E, H, K, R, N, Q, F, W, Y, P, or C; N468 replaced with D, E, H, K, R, A, G, I, L, S, T, M, V, F, W, Y, P, or C; R469 replaced with D, E, A, G, I, L, S, T, M, V, N, Q, F, W, Y, P, or C; Q470 replaced with D, E, H, K, R, A, G, I, L, S, T, M, V, F, W, Y, P, or C; C471 replaced with D, E, H, K, R, A, G, I, L, S, T, M, V, N, Q, F, W, Y, or P; Q472 replaced with D, E, H, K, R, A, G, I, L, S, T, M, V, F, W, Y, P, or C; F473 replaced with D, E, H, K, R, N, Q, A, G, I, L, S, T, M, V, P, or C; T474 replaced with D, E, H, K, R, N, Q, F, W, Y, P, or C; F475 replaced with D, E, H, K, R, N, Q, A, G, I, L, S, T, M, V, P, or C; G476 replaced with D, E, H, K, R, N, Q, F, W, Y, P, or C; E477 replaced with H, K, R, A, G, I, L, S, T, M, V, N, Q, F, W, Y, P, or C; D478 replaced with H, K, R, A, G, I, L, S, T, M, V, N, Q, F, W, Y, P, or C; S479 replaced with D, E, H, K, R, N, Q, F, W, Y, P, or C; K480 replaced with D, E, A, G, I, L, S, T, M, V, N, Q, F, W, Y, P, or C; H481 replaced with D, E, A, G, I, L, S, T, M, V, N, Q, F, W, Y, P, or C; C482 replaced with D, E, H, K, R, A, G, I, L, S, T, M, V, N, Q, F, W, Y, or P; P483 replaced with D, E, H, K, R, A, G, I, L, S, T, M, V, N, Q, F, W, Y, or C; D484replaced with H, K, R, A, G, I, L, S, T, M, V, N, Q, F, W, Y, P, or C; A485 replaced with D, E, H, K, R, N, Q, F, W, Y, P, or C; A486 replaced with D, E, H, K, R, N, Q, F, W, Y, P, or C; S487 replaced with D, E, H, K, R, N, Q, F, W, Y, P, or C; T488 replaced with D, E, H, K, R, N, Q, F, W, Y, P, or C; C489 replaced with D, E, H, K, R, A, G, I, L, S, T, M, V, N, Q, F, W, Y, or P; S490 replaced with D, E, H, K, R, N, Q, F, W, Y, P, or C; T491 replaced with D, E, H, K, R, N, Q, F, W, Y, P, or C; L492 replaced with D, E, H, K, R, N, Q, F, W, Y, P, or C; W493 replaced with D, E, H, K, R, N, Q, A, G, I, L, S, T, M, V, P, or C; C494 replaced with D, E, H, K, R, A, G, I, L, S, T, M, V, N, Q, F, W, Y, or P; T495 replaced with D, E, H, K, R, N, Q, F, W, Y, P, or C; G496 replaced with D, E, H, K, R, N, Q, F, W, Y, P, or C; T497 replaced with D, E, H, K, R, N, Q, F, W, Y, P, or C; S498 replaced with D, E, H, K, R, N, Q, F, W, Y, P, or C; G499 replaced with D, E, H, K, R, N, Q, F, W, Y, P, or C; G500 replaced with D, E, H, K, R, N, Q, F, W, Y, P, or C; V501 replaced with D, E, H, K, R, N, Q, F, W, Y, P, or C; L502 replaced with D, E, H, K, R, N, Q, F, W, Y, P, or C; V503 replaced with D, E, H, K, R, N, Q, F, W, Y, P, or C; C504 replaced with D, E, H, K, R, A, G, I, L, S, T, M, V, N, Q, F, W, Y, or P; Q505 replaced with D, E, H, K, R, A, G, I, L, S, T, M, V, F, W, Y, P, or C; T506 replaced with D, E, H, K, R, N, Q, F, W, Y, P, or C; K507 replaced with D, E, A, G, I, L, S, T, M, V, N, Q, F, W, Y, P, or C; H508 replaced with D, E, A, G, I, L, S, T, M, V, N, Q, F, W, Y, P, or C; F509 replaced with D, E, H, K, R, N, Q, A, G, I, L, S, T, M, V, P, or C; P510 replaced with D, E, H, K, R, A, G, I, L, S, T, M, V, N, Q, F, W, Y, or C; W511 replaced with D, E, H, K, R, N, Q, A, G, I, L, S, T, M, V, P, or C; A512 replaced with D, E, H, K, R, N, Q, F, W, Y, P, or C; D513 replaced with H, K, R, A, G, I, L, S, T, M, V, N, Q, F, W, Y, P, or C; G514 replaced with D, E, H, K, R, N, Q, F, W, Y, P, or C; T515 replaced with D, E, H, K, R, N, Q, F, W, Y, P, or C; S516 replaced with D, E, H, K, R, N, Q, F, W, Y, P, or C; C517 replaced with D, E, H, K, R, A, G, I, L, S, T, M, V, N, Q, F, W, Y, or P; G518 replaced with D, E, H, K, R, N, Q, F, W, Y, P, or C; E519 replaced with H, K, R, A, G, I, L, S, T, M, V, N, Q, F, W, Y, P, or C; G520 replaced with D, E, H, K, R, N, Q, F, W, Y, P, or C; K521 replaced with D, E, A, G, I, L, S, T, M, V, N, Q, F, W, Y, P, or C; W522 replaced with D, E, H, K, R, N, Q, A, G, I, L, S, T, M, V, P, or C; C523 replaced with D, E, H, K, R, A, G, I, L, S, T, M, V, N, Q, F, W, Y, or P; I524 replaced with D, E, H, K, R, N, Q, F, W, Y, P, or C; N525 replaced with D, E, H, K, R, A, G, I, L, S, T, M, V, F, W, Y, P, or C; G526 replaced with D, E, H, K, R, N, Q, F, W, Y, P, or C; K527 replaced with D, E, A, G, I, L, S, T, M, V, N, Q, F, W, Y, P, or C; C528 replaced with D, E, H, K, R, A, G, I, L, S, T, M, V, N, Q, F, W, Y, or P; V529 replaced with D, E, H, K, R, N, Q, F, W, Y, P, or —C; N530 replaced with D, E, H, K, R, A, G, I, L, S, T, M, V, F, W, Y, P, or C; K531 replaced with D, E, A, G, I, L, S, T, M, V, N, Q, F, W, Y, P, or C; T532 replaced with D, E, H, K, R, N, Q, F, W, Y, P, or C; D533 replaced with H, K, R, A, G, I, L, S, T, M, V, N, Q, F, W, Y, P, or C; R534 replaced with D, E, A, G, I, L, S, T, M, V, N, Q, F, W, Y, P, or C; K535 replaced with D, E, A, G, I, L, S, T, M, V, N, Q, F, W, Y, P, or C; H536 replaced with D, E, A, G, I, L, S, T, M, V, N, Q, F, W, Y, P, or C; F537 replaced with D, E, H, K, R, N, Q, A, G, I, L, S, T, M, V, P, or C; D538 replaced with H, K, R, A, G, I, L, S, T, M, V, N, Q, F, W, Y, P, or C; T539 replaced with D, E, H, K, R, N, Q, F, W, Y, P, or C; P540 replaced with D, E, H, K, R, A, G, I, L, S, T, M, V, N, Q, F, W, Y, or C; F541 replaced with D, E, H, K, R, N, Q, A, G, I, L, S, T, M, V, P, or C; H542 replaced with D, E, A, G, I, L, S, T, M, V, N, Q, F, W, Y, P, or C; G543 replaced with D, E, H, K, R, N, Q, F, W, Y, P, or C; S544 replaced with D, E, H, K, R, N, Q, F, W, Y, P, or C; W545 replaced with D, E, H, K, R, N, Q, A, G, I, L, S, T, M, V, P, or C; G546 replaced with D, E, H, K, R, N, Q, F, W, Y, P, or C; M547 replaced with D, E, H, K, R, N, Q, F, W, Y, P, or C; W548 replaced with D, E, H, K, R, N, Q, A, G, I, L, S, T, M, V, P, or C; G549 replaced with D, E, H, K, R, N, Q, F, W, Y, P, or C; P550 replaced with D, E, H, K, R, A, G, I, L, S, T, M, V, N, Q, F, W, Y, or C; W551 replaced with D, E, H, K, R, N, Q, A, G, I, L, S, T, M, V, P, or C; G552 replaced with D, E, H, K, R, N, Q, F, W, Y, P, or C; D553 replaced with H, K, R, A, G, I, L, S, T, M, V, N, Q, F, W, Y, P, or C; C554 replaced with D, E, H, K, R, A, G, I, L, S, T, M, V, N, Q, F, W, Y, or P; S555 replaced with D, E, H, K, R, N, Q, F, W, Y, P, or C; R556 replaced with D, E, A, G, I, L, S, T, M, V, N, Q, F, W, Y, P, or C; T557 replaced with D, E, H, K, R, N, Q, F, W, Y, P, or C; C558 replaced with D, E, H, K, R, A, G, I, L, S, T, M, V, N, Q, F, W, Y, or P; G559 replaced with D, E, H, K, R, N, Q, F, W, Y, P, or C; G560 replaced with D, E, H, K, R, N, Q, F, W, Y, P, or C; G561 replaced with D, E, H, K, R, N, Q, F, W, Y, P, or C; V562 replaced with D, E, H, K, R, N, Q, F, W, Y, P, or C; Q563 replaced with D, E, H, K, R, A, G, I, L, S, T, M, V, F, W, Y, P, or C; Y564 replaced with D, E, H, K, R, N, Q, A, G, I, L, S, T, M, V, P, or C; T565 replaced with D, E, H, K, R, N, Q, F, W, Y, P, or C; M566 replaced with D, E, H, K, R, N, Q, F, W, Y, P, or C; R567 replaced with D, E, A, G, I, L, S, T, M, V, N, Q, F, W, Y, P, or C; E568 replaced with H, K, R, A, G, I, L, S, T, M, V, N, Q, F, W, Y, P, or C; C569 replaced with D, E, H, K, R, A, G, I, L, S, T, M, V, N, Q, F, W, Y, or P; D570 replaced with H, K, R, A, G, I, L, S, T, M, V, N, Q, F, W, Y, P, or C; N571 replaced with D, E, H, K, R, A, G, I, L, S, T, M, V, F, W, Y, P, or C; P572 replaced with D, E, H, K, R, A, G, I, L, S, T, M, V, N, Q, F, W, Y, or C; V573 replaced with D, E, H, K, R, N, Q, F, W, Y, P, or C; P574 replaced with D, E, H, K, R, A, G, I, L, S, T, M, V, N, Q, F, W, Y, or C; K575 replaced with D, E, A, G, I, L, S, T, M, V, N, Q, F, W, Y, P, or C; N576 replaced with D, E, H, K, R, A, G, I, L, S, T, M, V, F, W, Y, P, or C; G577 replaced with D, E, H, K, R, N, Q, F, W, Y, P, or C; G578 replaced with D, E, H, K, R, N, Q, F, W, Y, P, or C; K579 replaced with D, E, A, G, I, L, S, T, M, V, N, Q, F, W, Y, P, or C; Y580 replaced with D, E, H, K, R, N, Q, A, G, I, L, S, T, M, V, P, or C; C581 replaced with D, E, H, K, R, A, G, I, L, S, T, M, V, N, Q, F, W, Y, or P; E582 replaced with H, K, R, A, G, I, L, S, T, M, V, N, Q, F, W, Y, P, or C; G583 replaced with D, E, H, K, R, N, Q, F, W, Y, P, or C; K584 replaced with D, E, A, G, I, L, S, T, M, V, N, Q, F, W, Y, P, or C; R585 replaced with D, E, A, G, I, L, S, T, M, V, N, Q, F, W, Y, P, or C; V586 replaced with D, E, H, K, R, N, Q, F, W, Y, P, or C; R587 replaced with D, E, A, G, I, L, S, T, M, V, N, Q, F, W, Y, P, or C; Y588 replaced with D, E, H, K, R, N, Q, A, G, I, L, S, T, M, V, P, or C; R589 replaced with D, E, A, G, I, L, S, T, M, V, N, Q, F, W, Y, P, or C; S590 replaced with D, E, H, K, R, N, Q, F, W, Y, P, or C; C591 replaced with D, E, H, K, R, A, G, I, L, S, T, M, V, N, Q, F, W, Y, or P; N592 replaced with D, E, H, K, R, A, G, I, L, S, T, M, V, F, W, Y, P, or C; L593 replaced with D, E, H, K, R, N, Q, F, W, Y, P, or C; E594 replaced with H, K, R, A, G, I, L, S, T, M, V, N, Q, F, W, Y, P, or C; D595 replaced with H, K, R, A, G, I, L, S, T, M, V, N, Q, F, W, Y, P, or C; C596 replaced with D, E, H, K, R, A, G, I, L, S, T, M, V, N, Q, F, W, Y, or P; P597 replaced with D, E, H, K, R, A, G, I, L, S, T, M, V, N, Q, F, W, Y, or C; D598 replaced with H, K, R, A, G, I, L, S, T, M, V, N, Q, F, W, Y, P, or C; N599 replaced with D, E, H, K, R, A, G, I, L, S, T, M, V, F, W, Y, P, or C; N600 replaced with D, E, H, K, R, A, G, I, L, S, T, M, V, F, W, Y, P, or C; G601 replaced with D, E, H, K, R, N, Q, F, W, Y, P, or C; K602 replaced with D, E, A, G, I, L, S, T, M, V, N, Q, F, W, Y, P, or C; T603 replaced with D, E, H, K, R, N, Q, F, W, Y, P, or C; F604 replaced with D, E, H, K, R, N, Q, A, G, I, L, S, T, M, V, P, or C; R605 replaced with D, E, A, G, I, L, S, T, M, V, N, Q, F, W, Y, P, or C; E606 replaced with H, K, R, A, G, I, L, S, T, M, V, N, Q, F, W, Y, P, or C; E607 replaced with H, K, R, A, G, I, L, S, T, M, V, N, Q, F, W, Y, P, or C; Q608 replaced with D, E, H, K, R, A, G, I, L, S, T, M, V, F, W, Y, P, or C; C609 replaced with D, E, H, K, R, A, G, I, L, S, T, M, V, N, Q, F, W, Y, or P; E610 replaced with H, K, R, A, G, I, L, S, T, M, V, N, Q, F, W, Y, P, or C; A611 replaced with D, E, H, K, R, N, Q, F, W, Y, P, or C; H612 replaced with D, E, A, G, I, L, S, T, M, V, N, Q, F, W, Y, P, or C; N613 replaced with D, E, H, K, R, A, G, I, L, S, T, M, V, F, W, Y, P, or C; E614 replaced with H, K, R, A, G, I, L, S, T, M, V, N, Q, F, W, Y, P, or C; F615 replaced with D, E, H, K, R, N, Q, A, G, I, L, S, T, M, V, P, or C; S616 replaced with D, E, H, K, R, N, Q, F, W, Y, P, or C; K617 replaced with D, E, A, G, I, L, S, T, M, V, N, Q, F, W, Y, P, or C; A618 replaced with D, E, H, K, R, N, Q, F, W, Y, P, or C; S619 replaced with D, E, H, K, R, N, Q, F, W, Y, P, or C; F620 replaced with D, E, H, K, R, N, Q, A, G, I, L, S, T, M, V, P, or C; G621 replaced with D, E, H, K, R, N, Q, F, W, Y, P, or C; S622 replaced with D, E, H, K, R, N, Q, F, W, Y, P, or C; G623 replaced with D, E, H, K, R, N, Q, F, W, Y, P, or C; P624 replaced with D, E, H, K, R, A, G, I, L, S, T, M, V, N, Q, F, W, Y, or C; A625 replaced with D, E, H, K, R, N, Q, F, W, Y, P, or C; V626 replaced with D, E, H, K, R, N, Q, F, W, Y, P, or C; E627 replaced with H, K, R, A, G, I, L, S, T, M, V, N, Q, F, W, Y, P, or C; W628 replaced with D, E, H, K, R, N, Q, A, G, I, L, S, T, M, V, P, or C; I629 replaced with D, E, H, K, R, N, Q, F, W, Y, P, or C; P630 replaced with D, E, H, K, R, A, G, I, L, S, T, M, V, N, Q, F, W, Y, or C; K631 replaced with D, E, A, G, I, L, S, T, M, V, N, Q, F, W, Y, P, or C; Y632 replaced with D, E, H, K, R, N, Q, A, G, I, L, S, T, M, V, P, or C; A633 replaced with D, E, H, K, R, N, Q, F, W, Y, P, or C; G634 replaced with D, E, H, K, R, N, Q, F, W, Y, P, or C; V635 replaced with D, E, H, K, R, N, Q, F, W, Y, P, or C; S636 replaced with D, E, H, K, R, N, Q, F, W, Y, P, or C; P637 replaced with D, E, H, K, R, A, G, I, L, S, T, M, V, N, Q, F, W, Y, or C; K638 replaced with D, E, A, G, I, L, S, T, M, V, N, Q, F, W, Y, P, or C; D639 replaced with H, K, R, A, G, I, L, S, T, M, V, N, Q, F, W, Y, P, or C; R640 replaced with D, E, A, G, I, L, S, T, M, V, N, Q, F, W, Y, P, or C; C641 replaced with D, E, H, K, R, A, G, I, L, S, T, M, V, N, Q, F, W, Y, or P; K642 replaced with D, E, A, G, I, L, S, T, M, V, N, Q, F, W, Y, P, or C; L643 replaced with D, E, H, K, R, N, Q, F, W, Y, P, or C; I644 replaced with D, E, H, K, R, N, Q, F, W, Y, P, or C; C645 replaced with D, E, H, K, R, A, G, I, L, S, T, M, V, N, Q, F, W, Y, or P; Q646 replaced with D, E, H, K, R, A, G, I, L, S, T, M, V, F, W, Y, P, or C; A647 replaced with D, E, H, K, R, N, Q, F, W, Y, P, or C; K648 replaced with D, E, A, G, I, L, S, T, M, V, N, Q, F, W, Y, P, or C; G649 replaced with D, E, H, K, R, N, Q, F, W, Y, P, or C; I650 replaced with D, E, H, K, R, N, Q, F, W, Y, P, or C; G651 replaced with D, E, H, K, R, N, Q, F, W, Y, P, or C; Y652 replaced with D, E, H, K, R, N, Q, A, G, I, L, S, T, M, V, P, or C; F653 replaced with D, E, H, K, R, N, Q, A, G, I, L, S, T, M, V, P, or C; F654 replaced with D, E, H, K, R, N, Q, A, G, I, L, S, T, M, V, P, or C; V655 replaced with D, E, H, K, R, N, Q, F, W, Y, P, or C; L656 replaced with D, E, H, K, R, N, Q, F, W, Y, P, or C; Q657 replaced with D, E, H, K, R, A, G, I, L, S, T, M, V, F, W, Y, P, or C; P658 replaced with D, E, H, K, R, A, G, I, L, S, T, M, V, N, Q, F, W, Y, or C; K659 replaced with D, E, A, G, I, L, S, T, M, V, N, Q, F, W, Y, P, or C; V660 replaced with D, E, H, K, R, N, Q, F, W, Y, P, or C; V661 replaced with D, E, H, K, R, N, Q, F, W, Y, P, or C; D662 replaced with H, K, R, A, G, I, L, S, T, M, V, N, Q, F, W, Y, P, or C; G663 replaced with D, E, H, K, R, N, Q, F, W, Y, P, or C; T664 replaced with D, E, H, K, R, N, Q, F, W, Y, P, or C; P665 replaced with D, E, H, K, R, A, G, I, L, S, T, M, V, N, Q, F, W, Y, or C; C666 replaced with D, E, H, K, R, A, G, I, L, S, T, M, V, N, Q, F, W, Y, or P; S667 replaced with D, E, H, K, R, N, Q, F, W, Y, P, or C; P668 replaced with D, E, H, K, R, A, G, I, L, S, T, M, V, N, Q, F, W, Y, or C; D669 replaced with H, K, R, A, G, I, L, S, T, M, V, N, Q, F, W, Y, P, or C; S670 replaced with D, E, H, K, R, N, Q, F, W, Y, P, or C; T671 replaced with D, E, H, K, R, N, Q, F, W, Y, P, or C; S672 replaced with D, E, H, K, R, N, Q, F, W, Y, P, or C; V673 replaced with D, E, H, K, R, N, Q, F, W, Y, P, or C; C674 replaced with D, E, H, K, R, A, G, I, L, S, T, M, V, N, Q, F, W, Y, or P; V675 replaced with D, E, H, K, R, N, Q, F, W, Y, P, or C; Q676 replaced with D, E, H, K, R, A, G, I, L, S, T, M, V, F, W, Y, P, or C; G677 replaced with D, E, H, K, R, N, Q, F, W, Y, P, or C; Q678 replaced with D, E, H, K, R, A, G I, L, S, T, M, V, F, W, Y, P, or C; C679 replaced with D, E, H, K, R, A, G, I, L, S, T, M, V, N, Q, F, W, Y, or P; V680 replaced with D, E, H, K, R, N, Q, F, W, Y, P, or C; K681 replaced with D, E, A, G, I, L, S, T, M, V, N, Q, F, W, Y, P, or C; A682 replaced with D, E, H, K, R, N, Q, F, W, Y, P, or C; G683 replaced with D, E, H, K, R, N, Q, F, W, Y, P, or C; C684 replaced with D, E, H, K, R, A, G, I, L, S, T, M, V, N, Q, F, W, Y, or P; D685 replaced with H, K, R, A, G, I, L, S, T, M, V, N, Q, F, W, Y, P, or C; R686 replaced with D, E, A, G, I, L, S, T, M, V, N, Q, F, W, Y, P, or C; I687 replaced with D, E, H, K, R, N, Q, F, W, Y, P, or C; I688 replaced with D, E, H, K, R, N, Q, F, W, Y, P, or C; D689 replaced with H, K, R, A, G, I, L, S, T, M, V, N, Q, F, W, Y, P, or C; S690 replaced with D, E, H, K, R, N, Q, F, W, Y, P, or C; K691 replaced with D, E, A, G, I, L, S, T, M, V, N, Q, F, W, Y, P, or C; K692 replaced with D, E, A, G, I, L, S, T, M, V, N, Q, F, W, Y, P, or C; K693 replaced with D, E, A, G, I, L, S, T, M, V, N, Q, F, W, Y, P, or C; F694 replaced with D, E, H, K, R, N, Q, A, G, I, L, S, T, M, V, P, or C; D695 replaced with H, K, R, A, G, I, L, S, T, M, V, N, Q, F, W, Y, P, or C; K696 replaced with D, E, A, G, I, L, S, T, M, V, N, Q, F, W, Y, P, or C; C697 replaced with D, E, H, K, R, A, G, I, L, S, T, M, V, N, Q, F, W, Y, or P; G698 replaced with D, E, H, K, R, N, Q, F, W, Y, P, or C; V699 replaced with D, E, H, K, R, N, Q, F, W, Y, P, or C; C700 replaced with D, E, H, K, R, A, G, I, L, S, T, M, V, N, Q, F, W, Y, or P; G701 replaced with D, E, H, K, R, N, Q, F, W, Y, P, or C; G702 replaced with D, E, H, K, R, N, Q, F, W, Y, P, or C; N703 replaced with D, E, H, K, R, A, G, I, L, S, T, M, V, F, W, Y, P, or C; G704 replaced with D, E, H, K, R, N, Q, F, W, Y, P, or C; S705 replaced with D, E, H, K, R, N, Q, F, W, Y, P, or C; T706 replaced with D, E, H, K, R, N, Q, F, W, Y, P, or C; C707 replaced with D, E, H, K, R, A, G, I, L, S, T, M, V, N, Q, F, W, Y, or P; K708 replaced with D, E, A, G, I, L, S, T, M, V, N, Q, F, W, Y, P, or C; K709 replaced with D, E, A, G, I, L, S, T, M, V, N, Q, F, W, Y, P, or C; I710replaced with D, E, H, K, R, N, Q, F, W, Y, P, or C; S711 replaced with D, E, H, K, R, N, Q, F, W, Y, P, or C; G712 replaced with D, E, H, K, R, N, Q, F, W, Y, P, or C; S713 replaced with D, E, H, K, R, N, Q, F, W, Y, P, or C; V714 replaced with D, E, H, K, R, N, Q, F, W, Y, P, or C; U715 replaced with D, E, H, K, R, N, Q, F, W, Y, P, or C; S716 replaced with D, E, H, K, R, N, Q, F, W, Y, P, or C; A717 replaced with D, E, H, K, R, N, Q, F, W, Y, P, or C; K718 replaced with D, E, A, G, I, L, S, T, M, V, N, Q, F, W, Y, P, or C; P719 replaced with D, E, H, K, R, A, G, I, L, S, T, M, V, N, Q, F, W, Y, or C; G720 replaced with D, E, H, K, R, N, Q, F, W, Y, P, or C; Y721 replaced with D, E, H, K, R, N, Q, A, G, I, L, S, T, M, V, P, or C; H722 replaced with D, E, A, G, I, L, S, T, M, V, N, Q, F, W, Y, P, or C; D723 replaced with H, K, R, A, G, I, L, S, T, M, V, N, Q, F, W, Y, P, or C; I724 replaced with D, E, H, K, R, N, Q, F, W, Y, P, or C; I725 replaced with D, E, H, K, R, N, Q, F, W, Y, P, or C; T726 replaced with D, E, H, K, R, N, Q, F, W, Y, P, or C; I727 replaced with D, E, H, K, R, N, Q, F, W, Y, P, or C; P728 replaced with D, E, H, K, R, A, G, I, L, S, T, M, V, N, Q, F, W, Y, or C; T729 replaced with D, E, H, K, R, N, Q, F, W, Y, P, or C; G730 replaced with D, E, H, K, R, N, Q, F, W, Y, P, or C; A731 replaced with D, E, H, K, R, N, Q, F, W, Y, P, or C; T732 replaced with D, E, H, K, R, N, Q, F, W, Y, P, or C; N733 replaced with D, E, H, K, R, A, G, I, L, S, T, M, V, F, W, Y, P, or C; I734 replaced with D, E, H, K, R, N, Q, F, W, Y, P, or C; E735 replaced with H, K, R, A, G, I, L, S, T, M, V, N, Q, F, W, Y, P, or C; V736 replaced with D, E, H, K, R, N, Q, F, W, Y, P, or C; K737 replaced with D, E, A, G, I, L, S, T, M, V, N, Q, F, W, Y, P, or C; Q738 replaced with D, E, H, K, R, A, G, I, L, S, T, M, V, F, W, Y, P, or C; R739 replaced with D, E, A, G, I, L, S, T, M, V, N, Q, F, W, Y, P, or C; N740 replaced with D, E, H, K, R, A, G, I, L, S, T, M, V, F, W, Y, P, or C; Q741 replaced with D, E, H, K, R, A, G, I, L, S, T, M, V, F, W, Y, P, or C; R742 replaced with D, E, A, G, I, L, S, T, M, V, N, Q, F, W, Y, P, or C; G743 replaced with D, E, H, K, R, N, Q, F, W, Y, P, or C; S744 replaced with D, E, H, K, R, N, Q, F, W, Y, P, or C; R745 replaced with D, E, A, G, I, L, S, T, M, V, N, Q, F, W, Y, P, or C; N746 replaced with D, E, H, K, R, A, G, I, L, S, T, M, V, F, W, Y, P, or C; N747 replaced with D, E, H, K, R, A, G, I, L, S, T, M, V, F, W, Y, P, or C; G748 replaced with D, E, H, K, R, N, Q, F, W, Y, P, or C; S749 replaced with D, E, H, K, R, N, Q, F, W, Y, P, or C; F750 replaced with D, E, H, K, R, N, Q, A, G, I, L, S, T, M, V, P, or C; L751 replaced with D, E, H, K, R, N, Q, F, W, Y, P, or C; A752 replaced with D, E, H, K, R, N, Q, F, W, Y, P, or C; I753 replaced with D, E, H, K, R, N, Q, F, W, Y, P, or C; K754 replaced with D, E, A, G, I, L, S, T, M, V, N, Q, F, W, Y, P, or C; A755 replaced with D, E, H, K, R, N, Q, F, W, Y, P, or C; A756 replaced with D, E, H, K, R, N, Q, F, W, Y, P, or C; D757 replaced with H, K, R, A, G, I, L, S, T, M, V, N, Q, F, W, Y, P, or C; G758 replaced with D, E, H, K, R, N, Q, F, W, Y, P, or C; T759 replaced with D, E, H, K, R, N, Q, F, W, Y, P, or C; Y760 replaced with D, E, H, K, R, N, Q, A, G, I, L, S, T, M, V, P, or C; I761 replaced with D, E, H, K, R, N, Q, F, W, Y, P, or C; L762 replaced with D, E, H, K, R, N, Q, F, W, Y, P, or C; N763 replaced with D, E, H, K, R, A, G, I, L, S, T, M, V, F, W, Y, P, or C; G764 replaced with D, E, H, K, R, N, Q, F, W, Y, P, or C; D765 replaced with H, K, R, A, G, I, L, S, T, M, V, N, Q, F, W, Y, P, or C; Y766 replaced with D, E, H, K, R, N, Q, A, G, I, L, S, T, M, V, P, or C; T767 replaced with D, E, H, K, R, N, Q, F, W, Y, P, or C; L768 replaced with D, E, H, K, R, N, Q, F, W, Y, P, or C; S769 replaced with D, E, H, K, R, N, Q, F, W, Y, P, or C; T770 replaced with D, E, H, K, R, N, Q, F, W, Y, P, or C; L771 replaced with D, E, H, K, R, N, Q, F, W, Y, P, or C; E772 replaced with H, K, R, A, G, I, L, S, T, M, V, N, Q, F, W, Y, P, or C; Q773 replaced with D, E, H, K, R, A, G, I, L, S, T, M, V, F, W, Y, P, or C; D774 replaced with H, K, R, A, G, I, L, S, T, M, V, N, Q, F, W, Y, P, or C; I775 replaced with D, E, H, K, R, N, Q, F, W, Y, P, or C; M776 replaced with D, E, H, K, R, N, Q, F, W, Y, P, or C; Y777 replaced with D, E, H, K, R, N, Q, A, G, I, L, S, T, M, V, P, or C; K778 replaced with D, E, A, G, I, L, S, T, M, V, N, Q, F, W, Y, P, or C; G779 replaced with D, E, H, K, R, N, Q, F, W, Y, P, or C; V780 replaced with D, E, H, K, R, N, Q, F, W, Y, P, or C; V781 replaced with D, E, H, K, R, N, Q, F, W, Y, P, or C; L782 replaced with D, E, H, K, R, N, Q, F, W, Y, P, or C; R783 replaced with D, E, A, G, I, L, S, T, M, V, N, Q, F, W, Y, P, or C; Y784 replaced with D, E, H, K, R, N, Q, A, G, I, L, S, T, M, V, P, or C; S785 replaced with D, E, H, K, R, N, Q, F, W, Y, P, or C; G786 replaced with D, E, H, K, R, N, Q, F, W, Y, P, or C; S787 replaced with D, E, H, K, R, N, Q, F, W, Y, P, or C; S788 replaced with D, E, H, K, R, N, Q, F, W, Y, P, or C; A789 replaced with D, E, H, K, R, N, Q, F, W, Y, P, or C; A790 replaced with D, E, H, K, R, N, Q, F, W, Y, P, or C; L791 replaced with D, E, H, K, R, N, Q, F, W, Y, P, or C; E792 replaced with H, K, R, A, G, I, L, S, T, M, V, N, Q, F, W, Y, P, or C; R793 replaced with D, E, A, G, I, L, S, T, M, V, N, Q, F, W, Y, P, or C; I794 replaced with D, E, H, K, R, N, Q, F, W, Y, P, or C; R795 replaced with D, E, A, G, I, L, S, T, M, V, N, Q, F, W, Y, P, or C; S796 replaced with D, E, H, K, R, N, Q, F, W, Y, P, or C; F797 replaced with D, E, H, K, R, N, Q, A, G, I, L, S, T, M, V, P, or C; S798 replaced with D, E, H, K, R, N, Q, F, W, Y, P, or C; P799 replaced with D, E, H, K, R, A, G, I, L, S, T, M, V, N, Q, F, W, Y, or C; L800 replaced with D, E, H, K, R, N, Q, F, W, Y, P, or C; K801 replaced with D, E, A, G, I, L, S, T, M, V, N, Q, F, W, Y, P, or C; E802 replaced with H, K, R, A, G, I, L, S, T, M, V, N, Q, F, W, Y, P, or C; P803 replaced with D, E, H, K, R, A, G, I, L, S, T, M, V, N, Q, F, W, Y, or C; L804 replaced with D, E, H, K, R, N, Q, F, W, Y, P, or C; T805 replaced with D, E, H, K, R, N, Q, F, W, Y, P, or C; I806 replaced with D, E, H, K, R, N, Q, F, W, Y, P, or C; Q807 replaced with D, E, H, K, R, A, G, I, L, S, T, M, V, F, W, Y, P, or C; V808 replaced with D, E, H, K, R, N, Q, F, W, Y, P, or C; L809 replaced with D, E, H, K, R, N, Q, F, W, Y, P, or C; T810 replaced with D, E, H, K, R, N, Q, F, W, Y, P, or C; V811 replaced with D, E, H, K, R, N, Q, F, W, Y, P, or C; G812 replaced with D, E, H, K, R, N, Q, F, W, Y, P, or C; N813 replaced with D, E, H, K, R, A, G, I, L, S, T, M, V, F, W, Y, P, or C; A814replaced with D, E, H, K, R, N, Q, F, W, Y, P, or C; L815 replaced with D, E, H, K, R, N, Q, F, W, Y, P, or C; R816 replaced with D, E, A, G, I, L, S, T, M, V, N, Q, F, W, Y, P, or C; P817 replaced with D, E, H, K, R, A, G, I, L, S, T, M, V, N, Q, F, W, Y, or C; K818 replaced with D, E, A, G, I, L, S, T, M, V, N, Q, F, W, Y, P, or C; I819 replaced with D, E, H, K, R, N, Q, F, W, Y, P, or C; K820 replaced with D, E, A, G, I, L, S, T, M, V, N, Q, F, W, Y, P, or C; Y821 replaced with D, E, H, K, R, N, Q, A, G, I, L, S, T, M, V, P, or C; T822 replaced with D, E, H, K, R, N, Q, F, W, Y, P, or C; Y823 replaced with D, E, H, K, R, N, Q, A, G, I, L, S, T, M, V, P, or C; F824 replaced with D, E, H, K, R, N, Q, A, G, I, L, S, T, M, V, P, or C; V825 replaced with D, E, H, K, R, N, Q, F, W, Y, P, or C; K826 replaced with D, E, A, G, I, L, S, T, M, V, N, Q, F, W, Y, P, or C; K827 replaced with D, E, A, G, I, L, S, T, M, V, N, Q, F, W, Y, P, or C; K828 replaced with D, E, A, G, I, L, S, T, M, V, N, Q, F, W, Y, P, or C; K829 replaced with D, E, A, G, I, L, S, T, M, V, N, Q, F, W, Y, P, or C; E830 replaced with H, K, R, A, G, I, L, S, T, M, V, N, Q, F, W, Y, P, or C; S831 replaced with D, E, H, K, R, N, Q, F, W, Y, P, or C; F832 replaced with D, E, H, K, R, N, Q, A, G, I, L, S, T, M, V, P, or C; N833 replaced with D, E, H, K, R, A, G, I, L, S, T, M, V, F, W, Y, P, or C; A834 replaced with D, E, H, K, R, N, Q, F, W, Y, P, or C; I835 replaced with D, E, H, K, R, N, Q, F, W, Y, P, or C; P836 replaced with D, E, H, K, R, A, G, I, L, S, T, M, V, N, Q, F, W, Y, or C; T837 replaced with D, E, H, K, R, N, Q, F, W, Y, P, or C; F838 replaced with D, E, H, K, R, N, Q, A, G, I, L, S, T, M, V, P, or C; S839 replaced with D, E, H, K, R, N, Q, F, W, Y, P, or C; A840 replaced with D, E, H, K, R, N, Q, F, W, Y, P, or C; W841 replaced with D, E, H, K, R, N, Q, A, G, I, L, S, T, M, V, P, or C; V842 replaced with D, E, H, K, R, N, Q, F, W, Y, P, or C; I843 replaced with D, E, H, K, R, N, Q, F, W, Y, P, or C; E844 replaced with H, K, R, A, G, I, L, S, T, M, V, N, Q, F, W, Y, P, or C; E845 replaced with H, K, R, A, G, I, L, S, T, M, V, N, Q, F, W, Y, P, or C; W846 replaced with D, E, H, K, R, N, Q, A, G, I, L, S, T, M, V, P, or C; G847 replaced with D, E, H, K, R, N, Q, F, W, Y, P, or C; E848 replaced with H, K, R, A, G, I, L, S, T, M, V, N, Q, F, W, Y, P, or C; C849 replaced with D, E, H, K, R, A, G, I, L, S, T, M, V, N, Q, F, W, Y, or P; S850 replaced with D, E, H, K, R, N, Q, F, W, Y, P, or C; K851 replaced with D, E, A, G, I, L, S, T, M, V, N, Q, F, W, Y, P, or C; S852 replaced with D, E, H, K, R, N, Q, F, W, Y, P, or C; C853 replaced with D, E, H, K, R, A, G, I, L, S, T, M, V, N, Q, F, W, Y, or P; E854 replaced with H, K, R, A, G, I, L, S, T, M, V, N, Q, F, W, Y, P, or C; L855 replaced with D, E, H, K, R, N, Q, F, W, Y, P, or C; G856 replaced with D, E, H, K, R, N, Q, F, W, Y, P, or C; W857 replaced with D, E, H, K, R, N, Q, A, G, I, L, S, T, M, V, P, or C; Q858 replaced with D, E, H, K, R, A, G, I, L, S, T, M, V, F, W, Y, P, or C; R859 replaced with D, E, A, G, I, L, S, T, M, V, N, Q, F, W, Y, P, or C; R860 replaced with D, E, A, G, I, L, S, T, M, V, N, Q, F, W, Y, P, or C; L861 replaced with D, E, H, K, R, N, Q, F, W, Y, P, or C; V862 replaced with D, E, H, K, R, N, Q, F, W, Y, P, or C; E863 replaced with H, K, R, A, G, I, L, S, T, M, V, N, Q, F, W, Y, P, or C; C864 replaced with D, E, H, K, R, A, G, I, L, S, T, M, V, N, Q, F, W, Y, or P; R865 replaced with D, E, A, G, I, L, S, T, M, V, N, Q, F, W, Y, P, or C; D866 replaced with H, K, R, A, G, I, L, S, T, M, V, N, Q, F, W, Y, P, or C; I867 replaced with D, E, H, K, R, N, Q, F, W, Y, P, or C; N868 replaced with D, E, H, K, R, A, G, I, L, S, T, M, V, F, W, Y, P, or C; G869 replaced with D, E, H, K, R, N, Q, F, W, Y, P, or C; Q870 replaced with D, E, H, K, R, A, G, I, L, S, T, M, V, F, W, Y, P, or C; P871 replaced with D, E, H, K, R, A, G, I, L, S, T, M, V, N, Q, F, W, Y, or C; A872 replaced with D, E, H, K, R, N, Q, F, W, Y, P, or C; S873 replaced with D, E, H, K, R, N, Q, F, W, Y, P, or C; E874 replaced with H, K, R, A, G, I, L, S, T, M, V, N, Q, F, W, Y, P, or C; C875 replaced with D, E, H, K, R, A, G, I, L, S, T, M, V, N, Q, F, W, Y, or P; A876 replaced with D, E, H, K, R, N, Q, F, W, Y, P, or C; K877 replaced with D, E, A, G, I, L, S, T, M, V, N, Q, F, W, Y, P, or C; E878 replaced with H, K, R, A, G, I, L, S, T, M, V, N, Q, F, W, Y, P, or C; V879 replaced with D, E, H, K, R, N, Q, F, W, Y, P, or C; K880 replaced with D, E, A, G, I, L, S, T, M, V, N, Q, F, W, Y, P, or C; P881 replaced with D, E, H, K, R, A, G, I, L, S, T, M, V, N, Q, F, W, Y, or C; A882 replaced with D, E, H, K, R, N, Q, F, W, Y, P, or C; S883 replaced with D, E, H, K, R, N, Q, F, W, Y, P, or C; T884 replaced with D, E, H, K, R, N, Q, F, W, Y, P, or C; R885 replaced with D, E, A, G, I, L, S, T, M, V, N, Q, F, W, Y, P, or C; P886 replaced with D, E, H, K, R, A, G, I, L, S, T, M, V, N, Q, F, W, Y, or C; C887 replaced with D, E, H, K, R, A, G, I, L, S, T, M, V, N, Q, F, W, Y, or P; A888 replaced with D, E, H, K, R, N, Q, F, W, Y, P, or C; D889 replaced with H, K, R, A, G, I, L, S, T, M, V, N, Q, F, W, Y, P, or C; H890 replaced with D, E, A, G, I, L, S, T, M, V, N, Q, F, W, Y, P, or C; P891 replaced with D, E, H, K, R, A, G, I, L, S, T, M, V, N, Q, F, W, Y, or C; C892 replaced with D, E, H, K, R, A, G, I, L, S, T, M, V, N, Q, F, W, Y, or P; P893 replaced with D, E, H, K, R, A, G, I, L, S, T, M, V, N, Q, F, W, Y, or C; Q894 replaced with D, E, H, K, R, A, G, I, L, S, T, M, V, F, W, Y, P, or C; W895 replaced with D, E, H, K, R, N, Q, A, G, I, L, S, T, M, V, P, or C; Q896 replaced with D, E, H, K, R, A, G, I, L, S, T, M, V, F, W, Y, P, or C; L897 replaced with D, E, H, K, R, N, Q, F, W, Y, P, or C; G898 replaced with D, E, H, K, R, N, Q, F, W, Y, P, or C; E899 replaced with H, K, R, A, G, I, L, S, T, M, V, N, Q, F, W, Y, P, or C; W900 replaced with D, E, H, K, R, N, Q, A, G, I, L, S, T, M, V, P, or C; S901 replaced with D, E, H, K, R, N, Q, F, W, Y, P, or C; S902 replaced with D, E, H, K, R, N, Q, F, W, Y, P, or C; C903 replaced with D, E, H, K, R, A, G, I, L, S, T, M, V, N, Q, F, W, Y, or P; S904 replaced with D, E, H, K, R, N, Q, F, W, Y, P, or C; K905 replaced with D, E, A, G, I, L, S, T, M, V, N, Q, F, W, Y, P, or C; T906 replaced with D, E, H, K, R, N, Q, F, W, Y, P, or C; C907 replaced with D, E, H, K, R, A, G, I, L, S, T, M, V, N, Q, F, W, Y, or P; G908 replaced with D, E, H, K, R, N, Q, F, W, Y, P, or C; K909 replaced with D, E, A, G, I, L, S, T, M, V, N, Q, F, W, Y, P, or C; G910 replaced with D, E, H, K, R, N, Q, F, W, Y, P, or C; Y911 replaced with D, E, H, K, R, N, Q, A, G, I, L, S, T, M, V, P, or C; K912 replaced with D, E, A, G, I, L, S, T, M, V, N, Q, F, W, Y, P, or C; K913 replaced with D, E, A, G, I, L, S, T, M, V, N, Q, F, W, Y, P, or C; R914 replaced with D, E, A, G, I, L, S, T, M, V, N, Q, F, W, Y, P, or C; S915 replaced with D, E, H, K, R, N, Q, F, W, Y, P, or C; L916 replaced with D, E, H, K, R, N, Q, F, W, Y, P, or C; K917 replaced with D, E, A, G, I, L, S, T, M, V, N, Q, F, W, Y, P, or C; C918 replaced with D, E, H, K, R, A, G, I, L, S, T, M, V, N, Q, F, W, Y, or P; L919 replaced with D, E, H, K, R, N, Q, F, W, Y, P, or C; S920 replaced with D, E, H, K, R, N, Q, F, W, Y, P, or C; H921 replaced with D, E, A, G, I, L, S, T, M, V, N, Q, F, W, Y, P, or C; D922 replaced with H, K, R, A, G, I, L, S, T, M, V, N, Q, F, W, Y, P, or C; G923 replaced with D, E, H, K, R, N, Q, F, W, Y, P, or C; G924 replaced with D, E, H, K, R, N, Q, F, W, Y, P, or C; V925 replaced with D, E, H, K, R, N, Q, F, W, Y, P, or C; L926 replaced with D, E, H, K, R, N, Q, F, W, Y, P, or C; S927 replaced with D, E, H, K, R, N, Q, F, W, Y, P, or C; H928 replaced with D, E, A, G, I, L, S, T, M, V, N, Q, F, W, Y, P, or C; E929 replaced with H, K, R, A, G, I, L, S, T, M, V, N, Q, F, W, Y, P, or C; S930 replaced with D, E, H, K, R, N, Q, F, W, Y, P, or C; C931 replaced with D, E, H, K, R, A, G, I, L, S, T, M, V, N, Q, F, W, Y, or P; D932 replaced with H, K, R, A, G, I, L, S, T, M, V, N, Q, F, W, Y, P, or C; P933 replaced with D, E, H, K, R, A, G, I, L, S, T, M, V, N, Q, F, W, Y, or C; L934 replaced with D, E, H, K, R, N, Q, F, W, Y, P, or C; K935 replaced with D, E, A, G, I, L, S, T, M, V, N, Q, F, W, Y, P, or C; K936 replaced with D, E, A, G, I, L, S, T, M, V, N, Q, F, W, Y, P, or C; P937 replaced with D, E, H, K, R, A, G, I, L, S, T, M, V, N, Q, F, W, Y, or C; K938 replaced with D, E, A, G, I, L, S, T, M, V, N, Q, F, W, Y, P, or C; H939 replaced with D, E, A, G, I, L, S, T, M, V, N, Q, F, W, Y, P, or C; F940 replaced with D, E, H, K, R, N, Q, A, G, I, L, S, T, M, V, P, or C; I941 replaced with D, E, H, K, R, N, Q, F, W, Y, P, or C; D942 replaced with H, K, R, A, G, I, L, S, T, M, V, N, Q, F, W, Y, P, or C; F943 replaced with D, E, H, K, R, N, Q, A, G, I, L, S, T, M, V, P, or C; C944 replaced with D, E, H, K, R, A, G, I, L, S, T, M, V, N, Q, F, W, Y, or P; T945 replaced with D, E, H, K, R, N, Q, F, W, Y, P, or C; M946 replaced with D, E, H, K, R, N, Q, F, W, Y, P, or C; A947 replaced with D, E, H, K, R, N, Q, F, W, Y, P, or C; E948 replaced with H, K, R, A, G, I, L, S, T, M, V, N, Q, F, W, Y, P, or C; C949 replaced with D, E, H, K, R, A, G, I, L, S, T, M, V, N, Q, F, W, Y, or P; S950 replaced with D, E, H, K, R, N, Q, F, W, Y, P, or C.

Also preferred are METH2 polypeptides with one or more of the following conservative amino acid substitutions: M1 replaced with A, G, I, L, S, T, or V; F2 replaced with W, or Y; A4 replaced with G, I, L, S, T, M, or V; A6 replaced with G, I, L, S, T, M, or V; A7 replaced with G, I, L, S, T, M, or V; R9 replaced with H, or K; W10 replaced with F, or Y; L11 replaced with A, G, I, S, T, M, or V; F13 replaced with W, or Y; L14 replaced with A, G, I, S, T, M, or V; L15 replaced with A, G, I, S, T, M, or V; L16 replaced with A, G, I, S, T, M, or V; L17 replaced with A, G, I, S, T, M, or V; L18 replaced with A, G, I, S, T, M, or V; L19 replaced with A, G, I, S, T, M, or V; L20 replaced with A, G, I, S, T, M, or V; L21 replaced with A, G, I, S, T, M, or V; L22 replaced with A, G, I, S, T, M, or V; L24 replaced with A, G, I, S, T, M, or V; A25 replaced with G, I, L, S, T, M, or V; R26 replaced with H, or K; G27 replaced with A, I, L, S, T, M, or V; A28 replaced with G, I, L, S, T, M, or V; A30 replaced with G, I, L, S, T, M, or V; R31 replaced with H, or K; A33 replaced with G, I, L, S, T, M, or V; A34 replaced with G, I, L, S, T, M, or V; G35 replaced with A, I, L, S, T, M, or V; G36 replaced with A, I, L, S, T, M, or V; Q37 replaced with N; A38 replaced with G, I, L, S, T, M, or V; S39 replaced with A, G, I, L, T, M, or V; E40 replaced with D; L41 replaced with A, G, I, S, T, M, or V; V42 replaced with A, G, I, L, S, T, or M; V43 replaced with A, G, I, L, S, T, or M; T45 replaced with A, G, I, L, S, M, or V; R46 replaced with H, or K; L47 replaced with A, G, I, S, T, M, or V; G49 replaced with A, I, L, S, T, M, or V; S50 replaced with A, G, I, L, T, M, or V; A51 replaced with G, I, L, S, T, M, or V; G52 replaced with A, I, L, S, T, M, or V; E53 replaced with D; L54 replaced with A, G, I, S, T, M, or V; A55 replaced with G, I, L, S, T, M, or V; L56 replaced with A, G, I, S, T, M, or V; H57 replaced with K, or R; L58 replaced with A, G, I, S, T, M, or V; S59 replaced with A, G, I, L, T, M, or V; A60 replaced with G, I, L, S, T, M, or V; F61 replaced with or Y; G62 replaced with A, I, L, S, T, M, or V; K63 replaced with H, or R; G64 replaced with A, I, L, S, T, M, or V; F65 replaced with W, or Y; V66 replaced with A, G, I, L, S, T, or M; L67 replaced with A, G, I, S, T, M, or V; R68 replaced with H, or K; L69 replaced with A, G, I, S, T, M, or V; A70 replaced with G, I, L, S, T, M, or V; D72 replaced with E; D73 replaced with E; S74 replaced with A, G, I, L, T, M, or V; F75 replaced with W, or Y; L76 replaced with A, G, I, S, T, M, or V; A77 replaced with G, I, L, S, T, M, or V; E79 replaced with D; F80 replaced with W, or Y; K81 replaced with H, or R; 182 replaced with A, G, L, S, T, M, or V; E83 replaced with D; R84 replaced with H, or K; L85 replaced with A, G, I, S, T, M, or V; G86 replaced with A, I, L, S, T, M, or V; G87 replaced with A, I, L, S, T, M, or V; S88 replaced with A, G, I, L, T, M, or V; G89 replaced with A, I, L, S, T, M, or V; R90 replaced with H, or K; A91 replaced with G, I, L, S, T, M, or V; T92 replaced with A, G, I, L, S, M, or V; G93 replaced with A, I, L, S, T, M, or V; G94 replaced with A, I, L, S, T, M, or V; E95 replaced with D; R96 replaced with H, or K; G97 replaced with A, I, L, S, T, M, or V; L98 replaced with A, G, I, S, T, M, or V; R99 replaced with H, or K; G100 replaced with A, I, L, S, T, M, or V; F102 replaced with W, or Y; F103 replaced with W, or Y; S104 replaced with A, G, I, L, T, M, or V; G105 replaced with A, I, L, S, T, M, or V; T106 replaced with A, G, I, L, S, M, or V; V107 replaced with A, G, I, L, S, T, or M; N108 replaced with Q; G109 replaced with A, I, L, S, T, M, or V; E110 replaced with D; E112 replaced with D; S113 replaced with A, G, I, L, T, M, or V; L114 replaced with A, G, I, S, T, M, or V; A115 replaced with G, I, L, S, T, M, or V; A116 replaced with G, I, L, S, T, M, or V; V117 replaced with A, G, I, L, S, T, or M; S118 replaced with A, G, I, L, T, M, or V; L19 replaced with A, G, I, S, T, M, or V; R121 replaced with H, or K; G122 replaced with A, I, L, S, T, M, or V; L123 replaced with A, G, I, S, T, M, or V; S 124 replaced with A, G, I, L, T, M, or V; G125 replaced with A, I, L, S, T, M, or V; S 126 replaced with A, G, I, L, T, M, or V; F127 replaced with W, or Y; L128 replaced with A, G, I, S, T, M, or V; L129 replaced with A, G, I, S, T, M, or V; D130 replaced with E; G131 replaced with A, I, L, S, T, M, or V; E132 replaced with D; E133 replaced with D; F134 replaced with W, or Y; T135 replaced with A, G, I, L, S, M, or V; I136 replaced with A, G, L, S, T, M, or V; Q137 replaced with N; Q139 replaced with N; G140 replaced with A, I, L, S, T, M, or V; A141 replaced with G, I, L, S, T, M, or V; G142 replaced with A, I, L, S, T, M, or V; G143 replaced with A, I, L, S, T, M, or V; S 144 replaced with A, G, I, L, T, M, or V; L145 replaced with A, G, I, S, T, M, or V; A146 replaced with G, I, L, S, T, M, or V; Q147 replaced with N; H149 replaced with K, or R; R150 replaced with H, or K; L151 replaced with A, G, I, S, T, M, or V; Q152 replaced with N; R153 replaced with H, or K; W154 replaced with F, or Y; G155 replaced with A, I, L, S, T, M, or V; A157 replaced with G, I, L, S, T, M, or V; G158 replaced with A, I, L, S, T, M, or V; A159 replaced with G, I, L, S, T, M, or V; R160 replaced with H, or K; L162 replaced with A, G, I, S, T, M, or V; R164 replaced with H, or K; G165 replaced with A, I, L, S, T, M, or V; E167 replaced with D; W168 replaced with F, or Y; E169 replaced with D; V170 replaced with A, G, I, L, S, T, or M; E171 replaced with D; T172 replaced with A, G, I, L, S, M, or V; G173 replaced with A, I, L, S, T, M, or V; E174 replaced with D; G175 replaced with A, I, L, S, T, M, or V; Q176 replaced with N; R177 replaced with H, or K; Q178 replaced with N; E179 replaced with D; R180 replaced with H, or K; G181 replaced with A, I, L, S, T, M, or V; D182 replaced with E; H183 replaced with K, or R; Q184 replaced with N; E185 replaced with D; D186 replaced with E; S187 replaced with A, G, I, L, T, M, or V; E188 replaced with D; E189 replaced with D; E190 replaced with D; S191 replaced with A, G, I, L, T, M, or V; Q192 replaced with N; E193 replaced with D; E194 replaced with D; E195 replaced with D; A196 replaced with G, I, L, S, T, M, or V; E197 replaced with D; G198 replaced with A, I, L, S, T, M, or V; A199 replaced with G, I, L, S, T, M, or V; S200 replaced with A, G, I, L, T, M, or V; E201 replaced with D; L206 replaced with A, G, I, S, T, M, or V; G207 replaced with A, I, L, S, T, M, or V; A208 replaced with G, I, L, S, T, M, or V; T209 replaced with A, G, I, L, S, M, or V; S210 replaced with A, G, I, L, T, M, or V; R211 replaced with H, or K; T212 replaced with A, G, I, L, S, M, or V; K213 replaced with H, or R; R214 replaced with H, or K; F215 replaced with W, or Y; V216 replaced with A, G, I, L, S, T, or M; S217 replaced with A, G, I, L, T, M, or V; E218 replaced with D; A219 replaced with G, I, L, S, T, M, or V; R220 replaced with H, or K; F221 replaced with W, or Y; V222 replaced with A, G, I, L, S, T, or M; E223 replaced with D; T224 replaced with A, G, I, L, S, M, or V; L225 replaced with A, G, I, S, T, M, or V; L226 replaced with A, G, I, S, T, M, or V; V227 replaced with A, G, I, L, S, T, or M; A228 replaced with G, I, L, S, T, M, or V; D229 replaced with E; A230 replaced with G, I, L, S, T, M, or V; S231 replaced with A, G, I, L, T, M, or V; M232 replaced with A, G, I, L, S, T, or V; A233 replaced with G, I, L, S, T, M, or V; A234 replaced with G, I, L, S, T, M, or V; F235 replaced with W, or Y; Y236 replaced with F, or W; G237 replaced with A, I, L, S, T, M, or V; A238 replaced with G, I, L, S, T, M, or V; D239 replaced with E; L240 replaced with A, G, I, S, T, M, or V; Q241 replaced with N; N242 replaced with Q; H243 replaced with K, or R; I244 replaced with A, G, L, S, T, M, or V; L245 replaced with A, G, I, S, T, M, or V; T246 replaced with A, G, I, L, S, M, or V; L247 replaced with A, G, I, S, T, M, or V; M248 replaced with A, G, I, L, S, T, or V; S249 replaced with A, G, I, L, T, M, or V; V250 replaced with A, G, I, L, S, T, or M; A251 replaced with G, I, L, S, T, M, or V; A252 replaced with G, I, L, S, T, M, or V; R253 replaced with H, or K; I254 replaced with A, G, L, S, T, M, or V; Y255 replaced with F, or W; K256 replaced with H, or R; H257 replaced with K, or R; S259 replaced with A, G, I, L, T, M, or V; I260 replaced with A, G, L, S, T, M, or V; K261 replaced with H, or R; N262 replaced with Q; S263 replaced with A, G, I, L, T, M, or V; I264 replaced with A, G, L, S, T, M, or V; N265 replaced with Q; L266 replaced with A, G, I, S, T, M, or V; M267 replaced with A, G, I, L, S, T, or V; V268 replaced with A, G, I, L, S, T, or M; V269 replaced with A, G, I, L, S, T, or M; K270 replaced with H, or R; V271 replaced with A, G, I, L, S, T, or M; L272 replaced with A, G, I, S, T, M, or V; I273 replaced with A, G, L, S, T, M, or V; V274 replaced with A, G, I, L, S, T, or M; E275 replaced with D; D276 replaced with E; E277 replaced with D; K278 replaced with H, or R; W279 replaced with F, or Y; G280 replaced with A, I, L, S, T, M, or V; E282 replaced with D; V283 replaced with A, G, I, L, S, T, or M; S284 replaced with A, G, I, L, T, M, or V; D285 replaced with E; N286 replaced with Q; G287 replaced with A, I, L, S, T, M, or V; G288 replaced with A, I, L, S, T, M, or V; L289 replaced with A, G, I, S, T, M, or V; T290 replaced with A, G, I, L, S, M, or V; L291 replaced with A, G, I, S, T, M, or V; R292 replaced with H, or K; N293 replaced with Q; F294 replaced with W, or Y; N296 replaced with Q; W297 replaced with F, or Y; Q298 replaced with N; R299 replaced with H, or K; R300 replaced with H, or K; F301 replaced with W, or Y; N302 replaced with Q; Q303 replaced with N; S305 replaced with A, G, I, L, T, M, or V; D306 replaced with E; R307 replaced with H, or K; H308 replaced with K, or R; E310 replaced with D; H311 replaced with K, or R; Y312 replaced with F, or W; D313 replaced with E; T314 replaced with A, G, I, L, S, M, or V; A315 replaced with G, I, L, S, T, M, or V; I316 replaced with A, G, L, S, T, M, or V; L317 replaced with A, G, I, S, T, M, or V; L318 replaced with A, G, I, S, T, M, or V; T319 replaced with A, G, I, L, S, M, or V; R320 replaced with H, or K; Q321 replaced with N; N322 replaced with Q; F323 replaced with W, or Y; G325 replaced with A, I, L, S, T, M, or V; Q326 replaced with N; E327 replaced with D; G328 replaced with A, I, L, S, T, M, or V; L329 replaced with A, G, I, S, T, M, or V; D331 replaced with E; T332 replaced with A, G, I, L, S, M, or V; L333 replaced with A, G, I, S, T, M, or V; G334 replaced with A, I, L, S, T, M, or V; V335 replaced with A, G, I, L, S, T, or M; A336 replaced with G, I, L, S, T, M, or V; D337 replaced with E; I338 replaced with A, G, L, S, T, M, or V; G339 replaced with A, I, L, S, T, M, or V; T340 replaced with A, G, I, L, S, M, or V; I341 replaced with A, G, L, S, T, M, or V; D343 replaced with E; N345 replaced with Q; K346 replaced with H, or R; S347 replaced with A, G, I, L, T, M, or V; S349 replaced with A, G, I, L, T, M, or V; V350 replaced with A, G, I, L, S, T, or M; I351 replaced with A, G, L, S, T, M, or V; E352 replaced with D; D353 replaced with E; E354 replaced with D; G355 replaced with A, I, L, S, T, M, or V; L356 replaced with A, G, I, S, T, M, or V; Q357 replaced with N; A358 replaced with G, I, L, S, T, M, or V; A359 replaced with G, I, L, S, T, M, or V; H360 replaced with K, or R; T361 replaced with A, G, I, L, S, M, or V; L362 replaced with A, G, I, S, T, M, or V; A363 replaced with G. I, L, S, T, M, or V; H364 replaced with K, or R; E365 replaced with D; L366 replaced with A, G, I, S, T, M, or V; G367 replaced with A, I, L, S, T, M, or V; H368 replaced with K, orR; V369 replaced with A, G, I, L, S, T, or M; L370 replaced with A, G, I, S, T, M, or V; S371 replaced with A, G, I, L, T, M, or V; M372 replaced with A, G, I, L, S, T, or V; H374 replaced with K, or R; D375 replaced with E; D376 replaced with E; S377 replaced with A, G, I, L, T, M, or V; K378 replaced with H, or R; T381 replaced with A, G, I, L, S, M, or V; R382 replaced with H, or K; L383 replaced with A, G, I, S, T, M, or V; F384 replaced with W, or Y; G385 replaced with A, I, L, S, T, M, or V; M387 replaced with A, G, I, L, S, T, or V; G388 replaced with A, I, L, S, T, M, or V; K389 replaced with H, or R; H390 replaced with K, or R; H391 replaced with K, or R; V392 replaced with A, G, I, L, S, T, or M; M393 replaced with A, G, I, L, S, T, or V; A394 replaced with G, I, L, S, T, M, or V; L396 replaced with A, G, I, S, T, M, or V; F397 replaced with W, or Y; V398 replaced with A, G, I, L, S, T, or M; H399 replaced with K, or R; L400 replaced with A, G, I, S, T, M, or V; N401 replaced with Q; Q402 replaced with N; T403 replaced with A, G, I, L, S, M, or V; L404 replaced with A, G, I, S, T, M, or V; W406 replaced with F, or Y; S407 replaced with A, G, I, L, T, M, or V; S410 replaced with A, G, I, L, T, M, or V; A411 replaced with G, I, L, S, T, M, or V; M412 replaced with A, G, I, L, S, T, or V; Y413 replaced with F, or W; L414 replaced with A, G, I, S, T, M, or V; T415 replaced with A, G, I, L, S, M, or V; E416 replaced with D; I417 replaced with A, G, I, S, T, M, or V; L418 replaced with A, G, I, S, T, M, or V; D419 replaced with E; G420 replaced with A, I, L, S, T, M, or V; G421 replaced with A, I, L, S, T, M, or V; H422 replaced with K, or R; G423 replaced with A, I, L, S, T, M, or V; D424 replaced with E; L426 replaced with A, G, I, S, T, M, or V; I427 replaced with A, G, I, S, T, M, or V; D428 replaced with E; A429 replaced with G, I, L, S, T, M, or V; G431 replaced with A, I, L, S, T, M, or V; A432 replaced with G, I, L, S, T, M, or V; A433 replaced with G, I, L, S, T, M, or V; L434 replaced with A, G, I, S, T, M, or V; I436 replaced with A, G, I, S, T, M, or V; T438 replaced with A, G, I, L, S, M, or V; G439 replaced with A, I, L, S, T, M, or V; L440 replaced with A, G, I, S, T, M, or V; G442 replaced with A, I, L, S, T, M, or V; R443 replaced with H, or K; M444 replaced with A, G, I, L, S, T, or V; A445 replaced with G, I, L, S, T, M, or V; I446 replaced with A, G, I, S, T, M, or V; Y447 replaced with F, or W; Q448 replaced with N; L449 replaced with A, G, I, S, T, M, or V; D450 replaced with E; Q451 replaced with N; Q452 replaced with N; R454 replaced with H, or K; Q455 replaced with N; I456 replaced with A, G, L, S, T, M, or V; F457 replaced with W, or Y; G458 replaced with A, I, L, S, T, M, or V; D460 replaced with E; F461 replaced with W, or Y; R462 replaced with H, or K; H463 replaced with K, or R; N466 replaced with Q; T467 replaced with A, G, I, L, S, M, or V; S468 replaced with A, G, I, L, T, M, or V; A469 replaced with G, I, L, S, T, M, or V; Q470 replaced with N; D471 replaced with E; V472 replaced with A, G, I, L, S, T, or M; A474 replaced with G, I, L, S, T, M, or V; Q475 replaced with N; L476 replaced with A, G, I, S, T, M, or V; W477 replaced with F, or Y; H479 replaced with K, or R; T480 replaced with A, G, I, L, S, M, or V; D481 replaced with E; G482 replaced with A, I, L, S, T, M, or V; A483 replaced with G, I, L, S, T, M, or V; E484 replaced with D; I486 replaced with A, G, I, S, T, M, or V; H488 replaced with K, or R; T489 replaced with A, G, I, L, S, M, or V; K490 replaced with H, or R; N491 replaced with Q; G492 replaced with A, I, L, S, T, M, or V; S493 replaced with A, G, I, L, T, M, or V; I494 replaced with A, G, I, S, T, M, or V; W496 replaced with F, or Y; A497 replaced with G, I, L, S, T, M, or V; D498 replaced with E; G499 replaced with A, I, L, S, T, M, or V; T500 replaced with A, G, I, L, S, M, or V; G503 replaced with A, I, L, S, T, M, or V; G505 replaced with A, I, L, S, T, M, or V; H506 replaced with K, or R; L507 replaced with A, G, I, S, T, M, or V; S509 replaced with A, G, I, L, T, M, or V; E510 replaced with D; G511 replaced with A, I, L, S, T, M, or V; S512 replaced with A, G, I, L, T, M, or V; L514 replaced with A, G, I, S, T, M, or V; E516 replaced with D; E517 replaced with D; E518 replaced with D; V519 replaced with A, G, I, L, S, T, or M; E520 replaced with D; R521 replaced with H, or K; K523 replaced with H, or R; V525 replaced with A, G, I, L, S, T, or M; V526 replaced with A, G, I, L, S, T, or M; D527 replaced with E; G528 replaced with A, I, L, S, T, M, or V; G529 replaced with A, I, L, S, T, M, or V; W530 replaced with F, or Y; A531 replaced with G, I, L, S, T, M, or V; W533 replaced with F, or Y; G534 replaced with A, I, L, S, T, M, or V; W536 replaced with F, or Y; G537 replaced with A, I, L, S, T, M, or V; E538 replaced with D; S540 replaced with A, G, I, L, T, M, or V; R541 replaced with H, or K; T542 replaced with A, G, I, L, S, M, or V; G544 replaced with A, I, L, S, T, M, or V; G545 replaced with A, I, L, S, T, M, or V; G546 replaced with A, I, L, S, T, M, or V; V547 replaced with A, G, I, L, S, T, or M; Q548 replaced with N; F549 replaced with W, or Y; S550 replaced with A, G, I, L, T, M, or V; H551 replaced with K, or R; R552 replaced with H, or K; E553 replaced with D; K555 replaced with H, or R; D556 replaced with E; E558 replaced with D; Q560 replaced with N; N561 replaced with Q; G562 replaced with A, I, L, S, T, M, or V; G563 replaced with A, I, L, S, T, M, or V; R564 replaced with H, or K; Y565 replaced with F, or W; L567 replaced with A, G, I, L, T, M, or V; G568 replaced with A, G, L, S, T, M, or V; R569 replaced with H, or K; R570 replaced with H, or K; A571 replaced with G, I, L, S, T, M, or V; K572 replaced with H, or R; Y573 replaced with F, or W; Q574 replaced with N; 575 replaced with A, G, I, L, T, M, or V; H577 replaced with K, or R; T578 replaced with A, G, I, L, S, M, or V; E579 replaced with D; E580 replaced with D; D584 replaced with E; G585 replaced with A, I, L, S, T, M, or V; K586 replaced with H, or R; S587 replaced with A, G, I, L, T, M, or V; F588 replaced with W, or Y; R589 replaced with H, or K; E590 replaced with D; Q591 replaced with N; Q592 replaced with N; E594 replaced with D; K595 replaced with H, or R; Y596 replaced with F, or W; N597 replaced with Q; A598 replaced with G, I, L, S, T, M, or V; Y599 replaced with F, or W; N600 replaced with Q; Y601 replaced with F, or W; T602 replaced with A, G, I, L, S, M, or V; D603 replaced with E; M604 replaced with A, G, I, L, S, T, or V; D605 replaced with E; G606 replaced with A, I, L, S, T, M, or V; N607 replaced with Q; L608 replaced with A, G, I, S, T, M, or V; L609 replaced with A, G, I, S, T, M, or V; Q610 replaced with N; W611 replaced with F, or Y; V612 replaced with A, G, I, L, S, T, or M; K614 replaced with H, or R; Y615 replaced with F, or W; A616 replaced with G, I, L, S, T, M, or V; G617 replaced with A, I, L, S, T, M, or V; V618 replaced with A, G, I, L, S, T, or M; S619 replaced with A, G, I, L, T, M, or V; R621 replaced with H, or K; D622 replaced with E; R623 replaced with H, or K; K625 replaced with H, or R; L626 replaced with A, G, I, S, T, M, or V; F627 replaced with W, or Y; R629 replaced with H, or K; A630 replaced with G, I, L, S, T, M, or V; R631 replaced with H, or K; G632 replaced with A, I, L, S, T, M, or V; R633 replaced with H, or K; S634 replaced with A, G, I, L, T, M, or V; E635 replaced with D; F636 replaced with W, or Y; K637 replaced with H, orR; V638 replaced with A, G, I, L, S, T, or M; F639 replaced with W, or Y; E640 replaced with D; A641 replaced with G, I, L, S, T, M, or V; K642 replaced with H, or R; V643 replaced with A, G, I, L, S, T, or M; I644 replaced with A, G, L, S, T, M, or V; D645 replaced with E; G646 replaced with A, I, L, S, T, M, or V; T647 replaced with A, G, I, L, S, M, or V; L648 replaced with A, G, I, S, T, M, or V; G650 replaced with A, I, L, S, T, M, or V; E652 replaced with D; T653 replaced with A, G, I, L, S, M, or V; L654 replaced with A, G, I, S, T, M, or V; A655 replaced with G, I, L, S, T, M, or V; I656 replaced with A, G, L, S, T, M, or V; V658 replaced with A, G, I, L, S, T, or M; R659 replaced with H, or K; G660 replaced with A, I, L, S, T, M, or V; Q661 replaced with N; V663 replaced with A, G, I, L, S, T, or M; K664 replaced with H, or R; A665 replaced with G, I, L, S, T, M, or V; G666 replaced with A, I, L, S, T, M, or V; D668 replaced with E; H669 replaced with K, or R; V670 replaced with A, G, I, L, S, T, or M; V671 replaced with A, G, I, L, S, T, or M; D672 replaced with E; S673 replaced with A, G, I, L, T, M, or V; R675 replaced with H, or K; K676 replaced with H, or R; L677 replaced with A, G, I, S, T, M, or V; D678 replaced with E; K679 replaced with H, or R; G681 replaced with A, I, L, S, T, M, or V; V682 replaced with A, G, I, L, S, T, or M; G684 replaced with A, I, L, S, T, M, or V; G685 replaced with A, I, L, S, T, M, or V; K686 replaced with H, or R; G687 replaced with A, I, L, S, T, M, or V; N688 replaced with Q; S689 replaced with A, G, I, L, T, M, or V; R691 replaced with H, or K; K692 replaced with H, or R; V693 replaced with A, G, I, L, S, T, or M; S694 replaced with A, G, I, L, T, M, or V; G695 replaced with A, I, L, S, T, M, or V; S696 replaced with A, G, I, L, T, M, or V; L697 replaced with A, G, I, S, T1 M, or V; T698 replaced with A, G, I, L, S, M, or V; T700 replaced with A, G, I, L, S, M, or V; N701 replaced with Q; Y702 replaced with F, or W; G703 replaced with A, I, L, S, T, M, or V; Y704 replaced with F, or W; N705 replaced with Q; D706 replaced with E; I707 replaced with A, G, L, S, T, M, or V; V708 replaced with A, G, I, L, S, T, or M; T709 replaced with A, G, I, L, S, M, or V; I710 replaced with A, G, L, S, T, M, or V; A712 replaced with G, I, L, S, T, M, or V; G713 replaced with A, I, L, S, T, M, or V; A714 replaced with G, I, L, S, T, M, or V; T715 replaced with A, G, I, L, S, M, or V; N716 replaced with Q; I717 replaced with A, G, L, S, T, M, or V; D718 replaced with E; V719 replaced with A, G, I, L, S, T, or M; K720 replaced with H, or R; Q721 replaced with N; R722 replaced with H, or K; S723 replaced with A, G, I, L, T, M, or V; H724 replaced with K, or R; G726 replaced with A, I, L, S, T, M, or V; V727 replaced with A, G, I, L, S, T, or M; Q728 replaced with N; N729 replaced with Q; D730 replaced with E; G731 replaced with A, I, L, S, T, M, or V; N732 replaced with Q; Y733 replaced with F, or W; L734 replaced with A, G, I, S, T, M, or V; A735 replaced with G, I, L, S, T, M, or V; L736 replaced with A, G, I, S, T, M, or V; K737 replaced with H, or R; T738 replaced with A, G, I, L, S, M, or V; A739 replaced with G, I, L, S, T, M, or V; D740 replaced with E; G741 replaced with A, I, L, S, T, M, or V; Q742 replaced with N; Y743 replaced with F, or W; L744 replaced with A, G, I, S, T, M, or V; L745 replaced with A, G, I, S, T, M, or V; N746 replaced with Q; G747 replaced with A, I, L, S, T, M, or V; N748 replaced with Q; L749 replaced with A, G, I, S, T, M, or V; A750 replaced with G, I, L, S, T, M, or V; I751 replaced with A, G, L, S, T, M, or V; S752 replaced with A, G, I, L, T, M, or V; A753 replaced with G, I, L, S, T, M, or V; I754 replaced with A, G, L, S, T, M, or V; E755 replaced with D; Q756 replaced with N; D757 replaced with E; I758 replaced with A, G, L, S, T, M, or V; L759 replaced with A, G, I, S, T, M, or V; V760 replaced with A, G, I, L, S, T, or M; K761 replaced with H, or R; G762 replaced with A, I, L, S, T, M, or V; T763 replaced with A, G, I, L, S, M, or V; I764 replaced with A, G, L, S, T, M, or V; L765 replaced with A, G, I, S, T, M, or V; K766 replaced with H, or R; Y767 replaced with F, or W; S768 replaced with A, G, I, L, T, M, or V; G769 replaced with A, I, L, S, T, M, or V; S770 replaced with A, G, I, L, T, M, or V; I771 replaced with A, G, L, S, T, M, or V; A772 replaced with G, I, L, S, T, M, or V; T773 replaced with A, G, I, L, S, M, or V; L774 replaced with A, G, I, S, T, M, or V; E775 replaced with D; R776 replaced with H, or K; L777 replaced with A, G, I, S, T, M, or V; Q778 replaced with N; S779 replaced with A, G, I, L, T, M, or V; F780 replaced with W, or Y; R781 replaced with H, or K; L783 replaced with A, G, I, S, T, M, or V; E785 replaced with D; L787 replaced with A, G, I, S, T, M, or V; T788 replaced with A, G, I, L, S, M, or V; V789 replaced with A, G, I, L, S, T, or M; Q790 replaced with N; L791 replaced with A, G, I, S, T, M, or V; L792 replaced with A, G, I, S, T, M, or V; T793 replaced with A, G, I, L, S, M, or V; V794 replaced with A, G, I, L, S, T, or M; G796 replaced with A, I, L, S, T, M, or V; E797 replaced with D; V798 replaced with A, G, I, L, S, T, or M; F799 replaced with W, or Y; K802 replaced with H, or R; V803 replaced with A, G, I, L, S, T, or M; K804 replaced with H, or R; Y805 replaced with F, or W; T806 replaced with A, G, I, L, S, M, or V; F807 replaced with W, or Y; F808 replaced with W, or Y; V809 replaced with A, G, I, L, S, T, or M; N811 replaced with Q; D812 replaced with E; V813 replaced with A, G, I, L, S, T, or M; D814 replaced with E; F815 replaced with W, or Y; S816 replaced with A, G, I, L, T, M, or V; M817 replaced with A, G, I, L, S, T, or V; Q818 replaced with N; S819 replaced with A, G, I, L, T, M, or V; S820 replaced with A, G, I, L, T, M, or V; K821 replaced with H, or R; E822 replaced with D; R823 replaced with H, or K; A824 replaced with G, I, L, S, T, M, or V; T825 replaced with A, G, I, L, S, M, or V; T826 replaced with A, G, I, L, S, M, or V; N827 replaced with Q; I828 replaced with A, G, L, S, T, M, or V; I829 replaced with A, G, L, S, T, M, or V; Q830 replaced with N; L832 replaced with A, G, I, S, T, M, or V; L833 replaced with A, G, I, S, T, M, or V; H834 replaced with K, or R; A835 replaced with G, I, L, S, T, M, or V; Q836 replaced with N; W837 replaced with F, or Y; V838 replaced with A, G, I, L, S, T, or M; L839 replaced with A, G, I, S, T, M, or V; G840 replaced with A, I, L, S, T, M, or V; D841 replaced with E; W842 replaced with F, or Y; S843 replaced with A, G, I, L, T, M, or V; E844 replaced with D; S846 replaced with A, G, I, L, T, M, or V; S847 replaced with A, G, I, L, T, M, or V; T848 replaced with A, G, I, L, S, M, or V; G850 replaced with A, I, L, S, T, M, or V; A851 replaced with G, I, L, S, T, M, or V; G852 replaced with A, I, L, S, T, M, or V; W853 replaced with F, or Y; Q854 replaced with N; R855 replaced with H, or K; R856 replaced with H, or K; T857 replaced with A, G, I, L, S, M, or V; V858 replaced with A, G, I, L, S, T, or M; E859 replaced with D; R861 replaced with H, or K; D862 replaced with E; S864 replaced with A, G, I, L, T, M, or V; G865 replaced with A, I, L, S, T, M, or V; Q866 replaced with N; A867 replaced with G, I, L, S, T, M, or V; S868 replaced with A, G, I, L, T, M, or V; A869 replaced with G, I, L, S, T, M, or V; T870 replaced with A, G, I, L, S, M, or V; N872 replaced with Q; K873 replaced with H, or R; A874 replaced with G, I, L, S, T, M, or V; L875 replaced with A, G, I, S, T, M, or V; K876 replaced with H, or R; E878 replaced with D; D879 replaced with E; A880 replaced with G, I, L, S, T, M, or V; K881 replaced with H, or R; E884 replaced with D; S885 replaced with A, G, I, L, T, M, or V; Q886 replaced with N; L887 replaced with A, G, I, S, T, M, or V; L890 replaced with A, G, I, S, T, M, or V.

Also preferred are METH2 polypeptides with one or more of the following conservative amino acid substitutions: M1 replaced with D, E, H, K, R, N, Q, F, W, Y, P, or C; F2 replaced with D, E, H, K, R, N, Q, A, G, I, L, S, T, M, V, P, or C; P3 replaced with D, E, H, K, R, A, G, I, L, S, T, M, V, N, Q, F, W, Y, or C; A4 replaced with D, E, H, K, R, N, Q, F, W, Y, P, or C; P5replaced with D, E, H, K, R, A, G, I, L, S, T, M, V, N, Q, F, W, Y, or C; A6 replaced with D, E, H, K, R, N, Q, F, W, Y, P, or C; A7 replaced with D, E, H, K, R, N, Q, F, W, Y, P, or C; P8 replaced with D, E, H, K, R, A, G, I, L, S, T, M, V, N, Q, F, W, Y, or C; R9 replaced with D, E, A, G, I, L, S, T, M, V, N, Q, F, W, Y, P, or C; W10 replaced with D, E, H, K, R, N, Q, A, G, I, L, S, T, M, V, P, or C; L11 replaced with D, E, H, K, R, N, Q, F, W, Y, P, or C; P12 replaced with D, E, H, K, R, A, G, I, L, S, T, M, V, N, Q, F, W, Y, or C; F13replaced with D, E, H, K, R, N, Q, A, G, I, L, S, T, M, V, P, or C; L14 replaced with D, E, H, K, R, N, Q, F, W, Y, P, or C; L15 replaced with D, E, H, K, R, N, Q, F, W, Y, P, or C; L16 replaced with D, E, H, K, R, N, Q, F, W, Y, P, or C; L17 replaced with D, E, H, K, R, N, Q, F, W, Y, P, or C; L18 replaced with D, E, H, K, R, N, Q, F, W, Y, P, or C; L19 replaced with D, E, H, K, R, N, Q, F, W, Y, P, or C; L20 replaced with D, E, H, K, R, N, Q, F, W, Y, P, or C; L21 replaced with D, E, H, K, R, N, Q, F, W, Y, P, or C; L22 replaced with D, E, H, K, R, N, Q, F, W, Y, P, or C; P23 replaced with D, E, H, K, R, A, G, I, L, S, T, M, V, N, Q, F, W, Y, or C; L24 replaced with D, E, H, K, R, N, Q, F, W, Y, P, or C; A25 replaced with D, E, H, K, R, N, Q, F, W, Y, P, or C; R26 replaced with D, E, A, G, I, L, S, T, M, V, N, Q, F, W, Y, P, or C; G27 replaced with D, E, H, K, R, N, Q, F, W, Y, P, or C; A28 replaced with D, E, H, K, R, N, Q, F, W, Y, P, or C; P29 replaced with D, E, H, K, R, A, G, I, L, S, T, M, V, N, Q, F, W, Y, or C; A30 replaced with D, E, H, K, R, N, Q, F, W, Y, P, or C; R31 replaced with D, E, A, G, I, L, S, T, M, V, N, Q, F, W, Y, P, or C; P32 replaced with D, E, H, K, R, A, G, I, L, S, T, M, V, N, Q, F, W, Y, or C; A33 replaced with D, E, H, K, R, N, Q, F, W, Y, P, or C; A34replaced with D, E, H, K, R, N, Q, F, W, Y, P, or C; G35 replaced with D, E, H, K, R, N, Q, F, W, Y, P, or C; G36 replaced with D, E, H, K, R, N, Q, F, W, Y, P, or C; Q37 replaced with D, E, H, K, R, A, G, I, L, S, T, M, V, F, W, Y, P, or C; A38 replaced with D, E, H, K, R, N, Q, F, W, Y, P, or C; S39 replaced with D, E, H, K, R, N, Q, F, W, Y, P, or C; E40 replaced with H, K, R, A, G, I, L, S, T, M, V, N, Q, F, W, Y, P, or C; 141 replaced with D, E, H, K, R, N, Q, F, W, Y, P, or C; V42 replaced with D, E, H, K, R, N, Q, F, W, Y, P, or C; V43 replaced with D, E, H, K, R, N, Q, F, W, Y, P, or C; P44 replaced with D, E, H, K, R, A, G, I, L, S, T, M, V, N, Q, F, W, Y, or C; T45 replaced with D, E, H, K, R, N, Q, F, W, Y, P, or C; R46 replaced with D, E, A, G, I, L, S, T, M, V, N, Q, F, W, Y, P, or C; L47 replaced with D, E, H, K, R, N, Q, F, W, Y, P, or C; P48 replaced with D, E, H, K, R, A, G, I, L, S, T, M, V, N, Q, F, W, Y, or C; G49 replaced with D, E, H, K, R, N, Q, F, W, Y, P, or C; S50 replaced with D, E, H, K, R, N, Q, F, W, Y, P, or C; A51 replaced with D, E, H, K, R, N, Q, F, W, Y, P, or C; G52 replaced with D, E, H, K, R, N, Q, F, W, Y, P, or C; E53 replaced with H, K, R, A, G, I, L, S, T, M, V, N, Q, F, W, Y, P, or C; L54 replaced with D, E, H, K, R, N, Q, F, W, Y, P, or C; A55 replaced with D, E, H, K, R, N, Q, F, W, Y, P, or C; L56 replaced with D, E, H, K, R, N, Q, F, W, Y, P, or C; H57 replaced with D, E, A, G, I, L, S, T, M, V, N, Q, F, W, Y, P, or C; L58 replaced with D, E, H, K, R, N, Q, F, W, Y, P, or C; S59 replaced with D, E, H, K, R, N, Q, F, W, Y, P, or C; A60 replaced with D, E, H, K, R, N, Q, F, W, Y, P, or C; F61 replaced with D, E, H, K, R, N, Q, A, G, I, L, S, T, M, V, P, or C; G62 replaced with D, E, H, K, R, N, Q, F, W, Y, P, or C; K63 replaced with D, E, A, G, I, L, S, T, M, V, N, Q, F, W, Y, P, or C; G64 replaced with D, E, H, K, R, N, Q, F, W, Y, P, or C; F65 replaced with D, E, H, K, R, N, Q, A, G, I, L, S, T, M, V, P, or C; V66 replaced with D, E, H, K, R, N, Q, F, W, Y, P, or C; L67 replaced with D, E, H, K, R, N, Q, F, W, Y, P, or C; R68 replaced with D, E, A, G, I, L, S, T, M, V, N, Q, F, W, Y, P, or C; L69 replaced with D, E, H, K, R, N, Q, F, W, Y, P, or C; A70 replaced with D, E, H, K, R, N, Q, F, W, Y, P, or C; P71 replaced with D, E, H, K, R, A, G, I, L, S, T, M, V, N, Q, F, W, Y, or C; D72 replaced with H, K, R, A, G, I, L, S, T, M, V, N, Q, F, W, Y, P, or C; D73 replaced with H, K, R, A, G, I, L, S, T, M, V, N, Q, F, W, Y, P, or C; S74 replaced with D, E, H, K, R, N, Q, F, W, Y, P, or C; F75 replaced with D, E, H, K, R, N, Q, A, G, I, L, S, T, M, V, P, or C; L76 replaced with D, E, H, K, R, N, Q, F, W, Y, P, or C; A77 replaced with D, E, H, K, R, N, Q, F, W, Y, P, or C; P78 replaced with D, E, H, K, R, A, G, I, L, S, T, M, V, N, Q, F, W, Y, or C; E79 replaced with H, K, R, A, G, I, L, S, T, M, V, N, Q, F, W, Y, P, or C; F80 replaced with D, E, H, K, R, N, Q, A, G, I, L, S, T, M, V, P, or C; K81replaced with D, E, A, G, I, L, S, T, M, V, N, Q, F, W, Y, P, or C; 182 replaced with D, E, H, K, R, N, Q, F, W, Y, P, or C; E83 replaced with H, K, R, A, G, I, L, S, T, M, V, N, Q, F, W, Y, P, or C; R84 replaced with D, E, A, G, I, L, S, T, M, V, N, Q, F, W, Y, P, or C; L85 replaced with D, E, H, K, R, N, Q, F, W, Y, P, or C; G86 replaced with D, E, H, K, R, N, Q, F, W, Y, P, or C; G87 replaced with D, E, H, K, R, N, Q, F, W, Y, P, or C; S88 replaced with D, E, H, K, R, N, Q, F, W, Y, P, or C; G89 replaced with D, E, H, K, R, N, Q, F, W, Y, P, or C; R90 replaced with D, E, A, G, I, L, S, T, M, V, N, Q, F, W, Y, P, or C; A91 replaced with D, E, H, K, R, N, Q, F, W, Y, P, or C; T92 replaced with D, E, H, K, R, N, Q, F, W, Y, P, or C; G93 replaced with D, E, H, K, R, N, Q, F, W, Y, P, or C; G94 replaced with D, E, H, K, R, N, Q, F, W, Y, P, or C; E95 replaced with H, K, R, A, G, I, L, S, T, M, V, N, Q, F, W, Y, P, or C; R96 replaced with D, E, A, G, I, L, S, T, M, V, N, Q, F, W, Y, P, or C; G97 replaced with D, E, H, K, R, N, Q, F, W, Y, P, or C; L98 replaced with D, E, H, K, R, N, Q, F, W, Y, P, or C; R99 replaced with D, E, A, G, I, L, S, T, M, V, N, Q, F, W, Y, P, or C; G100 replaced with D, E, H, K, R, N, Q, F, W, Y, P, or C; C101 replaced with D, E, H, K, R, A, G, I, L, S, T, M, V, N, Q, F, W, Y, or P; F102 replaced with D, E, H, K, R, N, Q, A, G, I, L, S, T, M, V, P, or C; F103 replaced with D, E, H, K, R, N, Q, A, G, I, L, S, T, M, V, P, or C; S104 replaced with D, E, H, K, R, N, Q, F, W, Y, P, or C; G105 replaced with D, E, H, K, R, N, Q, F, W, Y, P, or C; T106 replaced with D, E, H, K, R, N, Q, F, W, Y, P, or C; V107 replaced with D, E, H, K, R, N, Q, F, W, Y, P, or C; N108 replaced with D, E, H, K, R, A, G, I, L, S, T, M, V, F, W, Y, P, or C; G109 replaced with D, E, H, K, R, N, Q, F, W, Y, P, or C; E110 replaced with H, K, R, A, G, I, L, S, T, M, V, N, Q, F, W, Y, P, or C; P111 replaced with D, E, H, K, R, A, G, I, L, S, T, M, V, N, Q, F, W, Y, or C; El 12 replaced with H, K, R, A, G, I, L, S, T, M, V, N, Q, F, W, Y, P, or C; S113 replaced with D, E, H, K, R, N, Q, F, W, Y, P, or C; L14 replaced with D, E, H, K, R, N, Q, F, W, Y, P, or C; A115 replaced with D, E, H, K, R, N, Q, F, W, Y, P, or C; A116 replaced with D, E, H, K, R, N, Q, F, W, Y, P, or C; V117 replaced with D, E, H, K, R, N, Q, F, W, Y, P, or C; S118 replaced with D, E, H, K, R, N, Q, F, W, Y, P, or C; L119 replaced with D, E, H, K, R, N, Q, F, W, Y, P, or C; C120 replaced with D, E, H, K, R, A, G, I, L, S, T, M, V, N, Q, F, W, Y, or P; R121 replaced with D, E, A, G, I, L, S, T, M, V, N, Q, F, W, Y, P, or C; G122 replaced with D, E, H, K, R, N, Q, F, W, Y, P, or C; L123 replaced with D, E, H, K, R, N, Q, F, W, Y, P, or C; S124 replaced with D, E, H, K, R, N, Q, F, W, Y, P, or C; G125 replaced with D, E, H, K, R, N, Q, F, W, Y, P, or C; S126 replaced with D, E, H, K, R, N, Q, F, W, Y, P, or C; F127 replaced with D, E, H, K, R, N, Q, A, G, I, L, S, T, M, V, P, or C; L128 replaced with D, E, H, K, R, N, Q, F, W, Y, P, or C; L129 replaced with D, E, H, K, R, N, Q, F, W, Y, P, or C; D130 replaced with H, K, R, A, G, I, L, S, T, M, V, N, Q, F, W, Y, P, or C; G131 replaced with D, E, H, K, R, N, Q, F, W, Y, P, or C; E132 replaced with H, K, R, A, G, I, L, S, T, M, V, N, Q, F, W, Y, P, or C; E133replaced with H, K, R, A, G, I, L, S, T, M, V, N, Q, F, W, Y, P, or C; F134 replaced with D, E, H, K, R, N, Q, A, G, I, L, S, T, M, V, P, or C; T135 replaced with D, E, H, K, R, N, Q, F, W, Y, P, or C; I136 replaced with D, E, H, K, R, N, Q, F, W, Y, P, or C; Q137 replaced with D, E, H, K, R, A, G, I, L, S, T, M, V, F, W, Y, P, or C; P138 replaced with D, E, H, K, R, A, G, I, L, S, T, M, V, N, Q, F, W, Y, or C; Q139 replaced with D, E, H, K, R, A, G, I, L, S, T, M, V, F, W, Y, P, or C; G140 replaced with D, E, H, K, R, N, Q, F, W, Y, P, or C; A141 replaced with D, E, H, K, R, N, Q, F, W, Y, P, or C; G142 replaced with D, E, H, K, R, N, Q, F, W, Y, P, or C; G143 replaced with D, E, H, K, R, N, Q, F, W, Y, P, or C; S 144 replaced with D, E, H, K, R, N, Q, F, W, Y, P, or C; L145 replaced with D, E, H, K, R, N, Q, F, W, Y, P, or C; A146 replaced with D, E, H, K, R, N, Q, F, W, Y, P, or C; Q147 replaced with D, E, H, K, R, A, G, I, L, S, T, M, V, F, W, Y, P, or C; P148 replaced with D, E, H, K, R, A, G, I, L, S, T, M, V, N, Q, F, W, Y, or C; H149 replaced with D, E, A, G, I, L, S, T, M, V, N, Q, F, W, Y, P, or C; R150 replaced with D, E, A, G, I, L, S, T, M, V, N, Q, F, W, Y, P, or C; L151 replaced with D, E, H, K, R, N, Q, F, W, Y, P, or C; Q152 replaced with D, E, H, K, R, A, G, I, L, S, T, M, V, F, W, Y, P, or C; R153 replaced with D, E, A, G, I, L, S, T, M, V, N, Q, F, W, Y, P, or C; W154 replaced with D, E, H, K, R, N, Q, A, G, I, L, S, T, M, V, P, or C; G155 replaced with D, E, H, K, R, N, Q, F, W, Y, P, or C; P156 replaced with D, E, H, K, R, A, G, I, L, S, T, M, V, N, Q, F, W, Y, or C; A157 replaced with D, E, H, K, R, N, Q, F, W, Y, P, or C; G158 replaced with D, E, H, K, R, N, Q, F, W, Y, P, or C; A159 replaced with D, E, H, K, R, N, Q, F, W, Y, P, or C; R160 replaced with D, E, A, G, I, L, S, T, M, V, N, Q, F, W, Y, P, or C; P161 replaced with D, E, H, K, R, A, G, I, L, S, T, M, V, N, Q, F, W, Y, or C; L162 replaced with D, E, H, K, R, N, Q, F, W, Y, P, or C; P163 replaced with D, E, H, K, R, A, G, I, L, S, T, M, V, N, Q, F, W, Y, or C; R164 replaced with D, E, A, G, I, L, S, T, M, V, N, Q, F, W, Y, P, or C; G165 replaced with D, E, H, K, R, N, Q, F, W, Y, P, or C; P166 replaced with D, E, H, K, R, A, G, I, L, S, T, M, V, N, Q, F, W, Y, or C; E167 replaced with H, K, R, A, G, I, L, S, T, M, V, N, Q, F, W, Y, P, or C; W168 replaced with D, E, H, K, R, N, Q, A, G, I, L, S, T, M, V, P, or C; E169 replaced with H, K, R, A, G, I, L, S, T, M, V, N, Q, F, W, Y, P, or C; V170 replaced with D, E, H, K, R, N, Q, F, W, Y, P, or C; E171 replaced with H, K, R, A, G, I, L, S, T, M, V, N, Q, F, W, Y, P, or C; T172 replaced with D, E, H, K, R, N, Q, F, W, Y, P, or C; G173 replaced with D, E, H, K, R, N, Q, F, W, Y, P, or C; E174 replaced with H, K, R, A, G, I, L, S, T, M, V, N, Q, F, W, Y, P, or C; G175 replaced with D, E, H, K, R, N, Q, F, W, Y, P, or C; Q176 replaced with D, E, H, K, R, A, G, I, L, S, T, M, V, F, W, Y, P, or C; R177 replaced with D, E, A, G, I, L, S, T, M, V, N, Q, F, W, Y, P, or C; Q178 replaced with D, E, H, K, R, A, G, I, L, S, T, M, V, F, W, Y, P, or C; E179 replaced with H, K, R, A, G, I, L, S, T, M, V, N, Q, F, W, Y, P, or C; R180 replaced with D, E, A, G, I, L, S, T, M, V, N, Q, F, W, Y, P, or C; G181 replaced with D, E, H, K, R, N, Q, F, W, Y, P, or C; D182 replaced with H, K, R, A, G, I, L, S, T, M, V, N, Q, F, W, Y, P, or C; H183 replaced with D, E, A, G, I, L, S, T, M, V, N, Q, F, W, Y, P, or C; Q184 replaced with D, E, H, K, R, A, G, I, L, S, T, M, V, F, W, Y, P, or C; E185 replaced with H, K, R, A, G, I, L, S, T, M, V, N, Q, F, W, Y, P, or C; D186 replaced with H, K, R, A, G, I, L, S, T, M, V, N, Q, F, W, Y, P, or C; S187 replaced with D, E, H, K, R, N, Q, F, W, Y, P, or C; E188 replaced with H, K, R, A, G, I, L, S, T, M, V, N, Q, F, W, Y, P, or C; E189 replaced with H, K, R, A, G, I, L, S, T, M, V, N, Q, F, W, Y, P, or C; E190 replaced with H, K, R, A, G, I, L, S, T, M, V, N, Q, F, W, Y, P, or C; S 191 replaced with D, E, H, K, R, N, Q, F, W, Y, P, or C; Q192 replaced with D, E, H, K, R, A, G, I, L, S, T, M, V, F, W, Y, P, or C; E193 replaced with H, K, R, A, G, I, L, S, T, M, V, N, Q, F, W, Y, P, or C; E194 replaced with H, K, R, A, G, I, L, S, T, M, V, N, Q, F, W, Y, P, or C; E195 replaced with H, K, R, A, G, I, L, S, T, M, V, N, Q, F, W, Y, P, or C; A196 replaced with D, E, H, K, R, N, Q, F, W, Y, P, or C; E197 replaced with H, K, R, A, G, I, L, S, T, M, V, N, Q, F, W, Y, P, or C; G198 replaced with D, E, H, K, R, N, Q, F, W, Y, P, or C; A199 replaced with D, E, H, K, R, N, Q, F, W, Y, P, or C; S200 replaced with D, E, H, K, R, N, Q, F, W, Y, P, or C; E201 replaced with H, K, R, A, G, I, L, S, T, M, V, N, Q, F, W, Y, P, or C; P202 replaced with D, E, H, K, R, A, G, I, L, S, T, M, V, N, Q, F, W, Y, or C; P203 replaced with D, E, H, K, R, A, G, I, L, S, T, M, V, N, Q, F, W, Y, or C; P204 replaced with D, E, H, K, R, A, G, I, L, S, T, M, V, N, Q, F, W, Y, or C; P205 replaced with D, E, H, K, R, A, G, I, L, S, T, M, V, N, Q, F, W, Y, or C; L206 replaced with D, E, H, K, R, N, Q, F, W, Y, P, or C; G207 replaced with D, E, H, K, R, N, Q, F, W, Y, P, or C; A208 replaced with D, E, H, K, R, N, Q, F, W, Y, P, or C; T209 replaced with D, E, H, K, R, N, Q, F, W, Y, P, or C; S210 replaced with D, E, H, K, R, N, Q, F, W, Y, P, or C; R211 replaced with D, E, A, G, I, L, S, T, M, V, N, Q, F, W, Y, P, or C; T212 replaced with D, E, H, K, R, N, Q, F, W, Y, P, or C; K213 replaced with D, E, A, G, I, L, S, T, M, V, N, Q, F, W, Y, P, or C; R214 replaced with D, E, A, G, I, L, S, T, M, V, N, Q, F, W, Y, P, or C; F215 replaced with D, E, H, K, R, N, Q, A, G, I, L, S, T, M, V, P, or C; V216 replaced with D, E, H, K, R, N, Q, F, W, Y, P, or C; S217 replaced with D, E, H, K, R, N, Q, F, W, Y, P, or C; E218 replaced with H, K, R, A, G, I, L, S, T, M, V, N, Q, F, W, Y, P, or C; A219 replaced with D, E, H, K, R, N, Q, F, W, Y, P, or C; R220 replaced with D, E, A, G, I, L, S, T, M, V, N, Q, F, W, Y, P, or C; F221 replaced with D, E, H, K, R, N, Q, A, G, I, L, S, T, M, V, P, or C; V222 replaced with D, E, H, K, R, N, Q, F, W, Y, P, or C; E223 replaced with H, K, R, A, G, I, L, S, T, M, V, N, Q, F, W, Y, P, or C; T224 replaced with D, E, H, K, R, N, Q, F, W, Y, P, or C; L225 replaced with D, E, H, K, R, N, Q, F, W, Y, P, or C; L226 replaced with D, E, H, K, R, N, Q, F, W, Y, P, or C; V227 replaced with D, E, H, K, R, N, Q, F, W, Y, P, or C; A228 replaced with D, E, H, K, R, N, Q, F, W, Y, P, or C; D229 replaced with H, K, R, A, G, I, L, S, T, M, V, N, Q, F, W, Y, P, or C; A230 replaced with D, E, H, K, R, N, Q, F, W, Y, P, or C; S231 replaced with D, E, H, K, R, N, Q, F, W, Y, P, or C; M232 replaced with D, E, H, K, R, N, Q, F, W, Y, P, or C; A233 replaced with D, E, H, K, R, N, Q, F, W, Y, P, or C; A234 replaced with D, E, H, K, R, N, Q, F, W, Y, P, or C; F235 replaced with D, E, H, K, R, N, Q, A, G, I, L, S, T, M, V, P, or C; Y236 replaced with D, E, H, K, R, N, Q, A, G, I, L, S, T, M, V, P, or C; G237 replaced with D, E, H, K, R, N, Q, F, W, Y, P, or C; A238 replaced with D, E, H, K, R, N, Q, F, W, Y, P, or C; D239 replaced with H, K, R, A, G, I, L, S, T, M, V, N, Q, F, W, Y, P, or C; L240 replaced with D, E, H, K, R, N, Q, F, W, Y, P, or C; Q241 replaced with D, E, H, K, R, A, G, I, L, S, T, M, V, F, W, Y, P, or C; N242 replaced with D, E, H, K, R, A, G, I, L, S, T, M, V, F, W, Y, P, or C; H243 replaced with D, E, A, G, I, L, S, T, M, V, N, Q, F, W, Y, P, or C; I244 replaced with D, E, H, K, R, N, Q, F, W, Y, P, or C; L245 replaced with D, E, H, K, R, N, Q, F, W, Y, P, or C; T246 replaced with D, E, H, K, R, N, Q, F, W, Y, P, or C; L247 replaced with D, E, H, K, R, N, Q, F, W, Y, P, or C; M248 replaced with D, E, H, K, R, N, Q, F, W, Y, P, or C; S249replaced with D, E, H, K, R, N, Q, F, W, Y, P, or C; V250 replaced with D, E, H, K, R, N, Q, F, W, Y, P, or C; A251 replaced with D, E, H, K, R, N, Q, F, W, Y, P, or C; A252 replaced with D, E, H, K, R, N, Q, F, W, Y, P, or C; R253 replaced with D, E, A, G, I, L, S, T, M, V, N, Q, F, W, Y, P, or C; I254 replaced with D, E, H, K, R, N, Q, F, W, Y, P, or C; Y255 replaced with D, E, H, K, R, N, Q, A, G, I, L, S, T, M, V, P, or C; K256replaced with D, E, A, G, I, L, S, T, M, V, N, Q, F, W, Y, P, or C; H257 replaced with D, E, A, G, I, L, S, T, M, V, N, Q, F, W, Y, P, or C; P258 replaced with D, E, H, K, R, A, G, I, L, S, T, M, V, N, Q, F, W, Y, or C; S259 replaced with D, E, H, K, R, N, Q, F, W, Y, P, or C; I260 replaced with D, E, H, K, R, N, Q, F, W, Y, P, or C; K261 replaced with D, E, A, G, I, L, S, T, M, V, N, Q, F, W, Y, P, or C; N262 replaced with D, E, H, K, R, A, G, I, L, S, T, M, V, F, W, Y, P, or C; S263 replaced with D, E, H, K, R, N, Q, F, W, Y, P, or C; I264 replaced with D, E, H, K, R, N, Q, F, W, Y, P, or C; N265 replaced with D, E, H, K, R, A, G, I, L, S, T, M, V, F, W, Y, P, or C; L266 replaced with D, E, H, K, R, N, Q, F, W, Y, P, or C; M267 replaced with D, E, H, K, R, N, Q, F, W, Y, P, or C; V268 replaced with D, E, H, K, R, N, Q, F, W, Y, P, or C; V269 replaced with D, E, H, K, R, N, Q, F, W, Y, P, or C; K270 replaced with D, E, A, G, I, L, S, T, M, V, N, Q, F, W, Y, P, or C; V271 replaced with D, E, H, K, R, N, Q, F, W, Y, P, or C; L272 replaced with D, E, H, K, R, N, Q, F, W, Y, P, or C; I273 replaced with D, E, H, K, R, N, Q, F, W, Y, P, or C; V274 replaced with D, E, H, K, R, N, Q, F, W, Y, P, or C; E275 replaced with H, K, R, A, G, I, L, S, T, M, V, N, Q, F, W, Y, P, or C; D276 replaced with H, K, R, A, G, I, L, S, T, M, V, N, Q, F, W, Y, P, or C; E277 replaced with H, K, R, A, G, I, L, S, T, M, V, N, Q, F, W, Y, P, or C; K278 replaced with D, E, A, G, I, L, S, T, M, V, N, Q, F, W, Y, P, or C; W279 replaced with D, E, H, K, R, N, Q, A, G, I, L, S, T, M, V, P, or C; G280 replaced with D, E, H, K, R, N, Q, F, W, Y, P, or C; P281 replaced with D, E, H, K, R, A, G, I, L, S, T, M, V, N, Q, F, W, Y, or C; E282replaced with H, K, R, A, G, I, L, S, T, M, V, N, Q, F, W, Y, P, or C; V283 replaced with D, E, H, K, R, N, Q, F, W, Y, P, or C; S284 replaced with D, E, H, K, R, N, Q, F, W, Y, P, or C; D285 replaced with H, K, R, A, G, I, L, S, T, M, V, N, Q, F, W, Y, P, or C; N286 replaced with D, E, H, K, R, A, G, I, L, S, T, M, V, F, W, Y, P, or C; G287 replaced with D, E, H, K, R, N, Q, F, W, Y, P, or C; G288 replaced with D, E, H, K, R, N, Q, F, W, Y, P, or C; L289 replaced with D, E, H, K, R, N, Q, F, W, Y, P, or C; T290 replaced with D, E, H, K, R, N, Q, F, W, Y, P, or C; L291 replaced with D, E, H, K, R, N, Q, F, W, Y, P, or C; R292 replaced with D, E, A, G, I, L, S, T, M, V, N, Q, F, W, Y, P, or C; N293 replaced with D, E, H, K, R, A, G, I, L, S, T, M, V, F, W, Y, P, or C; F294 replaced with D, E, H, K, R, N, Q, A, G, I, L, S, T, M, V, P, or C; C295 replaced with D, E, H, K, R, A, G, I, L, S, T, M, V, N, Q, F, W, Y, or P; N296 replaced with D, E, H, K, R, A, G, I, L, S, T, M, V, F, W, Y, P, or C; W297 replaced with D, E, H, K, R, N, Q, A, G, I, L, S, T, M, V, P, or C; Q298 replaced with D, E, H, K, R, A, G, I, L, S, T, M, V, F, W, Y, P, or C; R299 replaced with D, E, A, G, I, L, S, T, M, V, N, Q, F, W, Y, P, or C; R300 replaced with D, E, A, G, I, L, S, T, M, V, N, Q, F, W, Y, P, or C; F301 replaced with D, E, H, K, R, N, Q, A, G, I, L, S, T, M, V, P, or C; N302 replaced with D, E, H, K, R, A, G, I, L, S, T, M, V, F, W, Y, P, or C; Q303 replaced with D, E, H, K, R, A, G, I, L, S, T, M, V, F, W, Y, P, or C; P304 replaced with D, E, H, K, R, A, G, I, L, S, T, M, V, N, Q, F, W, Y, or C; S305 replaced with D, E, H, K, R, N, Q, F, W, Y, P, or C; D306 replaced with H, K, R, A, G, I, L, S, T, M, V, N, Q, F, W, Y, P, or C; R307 replaced with D, E, A, G, I, L, S, T, M, V, N, Q, F, W, Y, P, or C; H308 replaced with D, E, A, G, I, L, S, T, M, V, N, Q, F, W, Y, P, or C; P309 replaced with D, E, H, K, R, A, G, I, L, S, T, M, V, N, Q, F, W, Y, or C; E310 replaced with H, K, R, A, G, I, L, S, T, M, V, N, Q, F, W, Y, P, or C; H311 replaced with D, E, A, G, I, L, S, T, M, V, N, Q, F, W, Y, P, or C; Y312 replaced with D, E, H, K, R, N, Q, A, G, I, L, S, T, M, V, P, or C; D313 replaced with H, K, R, A, G, I, L, S, T, M, V, N, Q, F, W, Y, P, or C; T314 replaced with D, E, H, K, R, N, Q, F, W, Y, P, or C; A315 replaced with D, E, H, K, R, N, Q, F, W, Y, P, or C; I316 replaced with D, E, H, K, R, N, Q, F, W, Y, P, or C; L317 replaced with D, E, H, K, R, N, Q, F, W, Y, P, or C; L318 replaced with D, E, H, K, R, N, Q, F, W, Y, P, or C; T319 replaced with D, E, H, K, R, N, Q, F, W, Y, P, or C; R320 replaced with D, E, A, G, I, L, S, T, M, V, N, Q, F, W, Y, P, or C; Q321 replaced with D, E, H, K, R, A, G, I, L, S, T, M, V, F, W, Y, P, or C; N322 replaced with D, E, H, K, R, A, G, I, L, S, T, M, V, F, W, Y, P, or C; F323 replaced with D, E, H, K, R, N, Q, A, G, I, L, S, T, M, V, P, or C; C324 replaced with D, E, H, K, R, A, G, I, L, S, T, M, V, N, Q, F, W, Y, or P; G325 replaced with D, E, H, K, R, N, Q, F, W, Y, P, or C; Q326 replaced with D, E, H, K, R, A, G, I, L, S, T, M, V, F, W, Y, P, or C; E327 replaced with H, K, R, A, G, I, L, S, T, M, V, N, Q, F, W, Y, P, or C; G328 replaced with D, E, H, K, R, N, Q, F, W, Y, P, or C; L329 replaced with D, E, H, K, R, N, Q, F, W, Y, P, or C; C330 replaced with D, E, H, K, R, A, G, I, L, S, T, M, V, N, Q, F, W, Y, or P; D331 replaced with H, K, R, A, G, I, L, S, T, M, V, N, Q, F, W, Y, P, or C; T332 replaced with D, E, H, K, R, N, Q, F, W, Y, P, or C; L333 replaced with D, E, H, K, R, N, Q, F, W, Y, P, or C; G334 replaced with D, E, H, K, R, N, Q, F, W, Y, P, or C; V335 replaced with D, E, H, K, R, N, Q, F, W, Y, P, or C; A336 replaced with D, E, H, K, R, N, Q, F, W, Y, P, or C; D337 replaced with H, K, R, A, G, I, L, S, T, M, V, N, Q, F, W, Y, P, or C; I338 replaced with D, E, H, K, R, N, Q, F, W, Y, P, or C; G339 replaced with D, E, H, K, R, N, Q, F, W, Y, P, or C; T340 replaced with D, E, H, K, R, N, Q, F, W, Y, P, or C; I341 replaced with D, E, H, K, R, N, Q, F, W, Y, P, or C; C342 replaced with D, E, H, K, R, A, G, I, L, S, T, M, V, N, Q, F, W, Y, or P; D343 replaced with H, K, R, A, G, I, L, S, T, M, V, N, Q, F, W, Y, P, or C; P344 replaced with D, E, H, K, R, A, G, I, L, S, T, M, V, N, Q, F, W, Y, or C; N345 replaced with D, E, H, K, R, A, G, I, L, S, T, M, V, F, W, Y, P, or C; K346 replaced with D, E, A, G, I, L, S, T, M, V, N, Q, F, W, Y, P, or C; S347 replaced with D, E, H, K, R, N, Q, F, W, Y, P, or C; C348 replaced with D, E, H, K, R, A, G, I, L, S, T, M, V, N, Q, F, W, Y, or P; S349 replaced with D, E, H, K, R, N, Q, F, W, Y, P, or C; V350 replaced with D, E, H, K, R, N, Q, F, W, Y, P, or C; ¹³⁵I replaced with D, E, H, K, R, N, Q, F, W, Y, P, or C; E352 replaced with H, K, R, A, G, I, L, S, T, M, V, N, Q, F, W, Y, P, or C; D353 replaced with H, K, R, A, G, I, L, S, T, M, V, N, Q, F, W, Y, P, or C; E354 replaced with H, K, R, A, G, I, L, S, T, M, V, N, Q, F, W, Y, P, or C; G355 replaced with D, E, H, K, R, N, Q, F, W, Y, P, or C; L356 replaced with D, E, H, K, R, N, Q, F, W, Y, P, or C; Q357 replaced with D, E, H, K, R, A, G, I, L, S, T, M, V, F, W, Y, P, or C; A358 replaced with D, E, H, K, R, N, Q, F, W, Y, P, or C; A359 replaced with D, E, H, K, R, N, Q, F, W, Y, P, or C; H360 replaced with D, E, A, G, I, L, S, T, M, V, N, Q, F, W, Y, P, or C; T361 replaced with D, E, H, K, R, N, Q, F, W, Y, P, or C; L362 replaced with D, E, H, K, R, N, Q, F, W, Y, P, or C; A363 replaced with D, E, H, K, R, N, Q, F, W, Y, P, or C; H364 replaced with D, E, A, G, I, L, S, T, M, V, N, Q, F, W, Y, P, or C; E365 replaced with H, K, R, A, G, I, L, S, T, M, V, N, Q, F, W, Y, P, or C; L366 replaced with D, E, H, K, R, N, Q, F, W, Y, P, or C; G367 replaced with D, E, H, K, R, N, Q, F, W, Y, P, or C; H368 replaced with D, E, A, G, I, L, S, T, M, V, N, Q, F, W, Y, P, or C; V369 replaced with D, E, H, K, R, N, Q, F, W, Y, P, or C; L370 replaced with D, E, H, K, R, N, Q, F, W, Y, P, or C; S371 replaced with D, E, H, K, R, N, Q, F, W, Y, P, or C; M372 replaced with D, E, H, K, R, N, Q, F, W, Y, P, or C; P373 replaced with D, E, H, K, R, A, G, I, L, S, T, M, V, N, Q, F, W, Y, or C; H374 replaced with D, E, A, G, I, L, S, T, M, V, N, Q, F, W, Y, P, or C; D375 replaced with H, K, R, A, G, I, L, S, T, M, V, N, Q, F, W, Y, P, or C; D376 replaced with H, K, R, A, G, I, L, S, T, M, V, N, Q, F, W, Y, P, or C; S377 replaced with D, E, H, K, R, N, Q, F, W, Y, P, or C; K378 replaced with D, E, A, G, I, L, S, T, M, V, N, Q, F, W, Y, P, or C; P379 replaced with D, E, H, K, R, A, G, I, L, S, T, M, V, N, Q, F, W, Y, or C; C380 replaced with D, E, H, K, R, A, G, I, L, S, T, M, V, N, Q, F, W, Y, or P; T381 replaced with D, E, H, K, R, N, Q, F, W, Y, P, or C; R382 replaced with D, E, A, G, I, L, S, T, M, V, N, Q, F, W, Y, P, or C; L383 replaced with D, E, H, K, R, N, Q, F, W, Y, P, or C; F384 replaced with D, E, H, K, R, N, Q, A, G, I, L, S, T, M, V, P, or C; G385 replaced with D, E, H, K, R, N, Q, F, W, Y, P, or C; P386 replaced with D, E, H, K, R, A, G, I, L, S, T, M, V, N, Q, F, W, Y, or C; M387 replaced with D, E, H, K, R, N, Q, F, W, Y, P, or C; G388 replaced with D, E, H, K, R, N, Q, F, W, Y, P, or C; K389 replaced with D, E, A, G, I, L, S, T, M, V, N, Q, F, W, Y, P, or C; H390 replaced with D, E, A, G, I, L, S, T, M, V, N, Q, F, W, Y, P, or C; H391 replaced with D, E, A, G, I, L, S, T, M, V, N, Q, F, W, Y, P, or C; V392 replaced with D, E, H, K, R, N, Q, F, W, Y, P, or C; M393 replaced with D, E, H, K, R, N, Q, F, W, Y, P, or C; A394 replaced with D, E, H, K, R, N, Q, F, W, Y, P, or C; P395 replaced with D, E, H, K, R, A, G, I, L, S, T, M, V, N, Q, F, W, Y, or C; L396 replaced with D, E, H, K, R, N, Q, F, W, Y, P, or C; F397 replaced with D, E, H, K, R, N, Q, A, G, I, L, S, T, M, V, P, or C; V398 replaced with D, E, H, K, R, N, Q, F, W, Y, P, or C; H399 replaced with D, E, A, G, I, L, S, T, M, V, N, Q, F, W, Y, P, or C; L400 replaced with D, E, H, K, R, N, Q, F, W, Y, P, or C; N401 replaced with D, E, H, K, R, A, G, I, L, S, T, M, V, F, W, Y, P, or C; Q402 replaced with D, E, H, K, R, A, G, I, L, S, T, M, V, F, W, Y, P, or C; T403 replaced with D, E, H, K, R, N, Q, F, W, Y, P, or C; L404 replaced with D, E, H, K, R, N, Q, F, W, Y, P, or C; P405 replaced with D, E, H, K, R, A, G, I, L, S, T, M, V, N, Q, F, W, Y, or C; W406 replaced with D, E, H, K, R, N, Q, A, G, I, L, S, T, M, V, P, or C; S407 replaced with D, E, H, K, R, N, Q, F, W, Y, P, or C; P408 replaced with D, E, H, K, R, A, G, I, L, S, T, M, V, N, Q, F, W, Y, or C; C409 replaced with D, E, H, K, R, A, G, I, L, S, T, M, V, N, Q, F, W, Y, or P; S410 replaced with D, E, H, K, R, N, Q, F, W, Y, P, or C; A411 replaced with D, E, H, K, R, N, Q, F, W, Y, P, or C; M412 replaced with D, E, H, K, R, N, Q, F, W, Y, P, or C; Y413 replaced with D, E, H, K, R, N, Q, A, G, I, L, S, T, M, V, P, or C; L414 replaced with D, E, H, K, R, N, Q, F, W, Y, P, or C; T415 replaced with D, E, H, K, R, N, Q, F, W, Y, P, or C; E416 replaced with H, K, R, A, G, I, L, S, T, M, V, N, Q, F, W, Y, P, or C; L417 replaced with D, E, H, K, R, N, Q, F, W, Y, P, or C; 418 replaced with D, E, H, K, R, N, Q, F, W, Y, P, or C; D419 replaced with H, K, R, A, G, I, L, S, T, M, V, N, Q, F, W, Y, P, or C; G420 replaced with D, E, H, K, R, N, Q, F, W, Y, P, or C; G421 replaced with D, E, H, K, R, N, Q, F, W, Y, P, or C; H422 replaced with D, E, A, G, I, L, S, T, M, V, N, Q, F, W, Y, P, or C; G423 replaced with D, E, H, K, R, N, Q, F, W, Y, P, or C; D424 replaced with H, K, R, A, G, I, L, S, T, M, V, N, Q, F, W, Y, P, or C; C425 replaced with D, E, H, K, R, A, G, I, L, S, T, M, V, N, Q, F, W, Y, or P; L426 replaced with D, E, H, K, R, N, Q, F, W, Y, P, or C; L427 replaced with D, E, H, K, R, N, Q, F, W, Y, P, or C; D428 replaced with H, K, R, A, G, I, L, S, T, M, V, N, Q, F, W, Y, P, or C; A429 replaced with D, E, H, K, R, N, Q, F, W, Y, P, or C; P430 replaced with D, E, H, K, R, A, G, I, L, S, T, M, V, N, Q, F, W, Y, or C; G431 replaced with D, E, H, K, R, N, Q, F, W, Y, P, or C; A432 replaced with D, E, H, K, R, N, Q, F, W, Y, P, or C; A433 replaced with D, E, H, K, R, N, Q, F, W, Y, P, or C; L434 replaced with D, E, H, K, R, N, Q, F, W, Y, P, or C; P435 replaced with D, E, H, K, R, A, G, I, L, S, T, M, V, N, Q, F, W, Y, or C; I436 replaced with D, E, H, K, R, N, Q, F, W, Y, P, or C; P437 replaced with D, E, H, K, R, A, G, I, L, S, T, M, V, N, Q, F, W, Y, or C; T438 replaced with D, E, H, K, R, N, Q, F, W, Y, P, or C; G439 replaced with D, E, H, K, R, N, Q, F, W, Y, P, or C; L440 replaced with D, E, H, K, R, N, Q, F, W, Y, P, or C; P441 replaced with D, E, H, K, R, A, G, I, L, S, T, M, V, N, Q, F, W, Y, or C; G442 replaced with D, E, H, K, R, N, Q, F, W, Y, P, or C; R443 replaced with D, E, A, G, I, L, S, T, M, V, N, Q, F, W, Y, P, or C; M444 replaced with D, E, H, K, R, N, Q, F, W, Y, P, or C; A445 replaced with D, E, H, K, R, N, Q, F, W, Y, P, or C; L446 replaced with D, E, H, K, R, N, Q, F, W, Y, P, or C; Y447 replaced with D, E, H, K, R, N, Q, A, G, I, L, S, T, M, V, P, or C; Q448 replaced with D, E, H, K, R, A, G, I, L, S, T, M, V, F, W, Y, P, or C; L449 replaced with D, E, H, K, R, N, Q, F, W, Y, P, or C; D450 replaced with H, K, R, A, G, I, L, S, T, M, V, N, Q, F, W, Y, P, or C; Q451 replaced with D, E, H, K, R, A, G, I, L, S, T, M, V, F, W, Y, P, or C; Q452 replaced with D, E, H, K, R, A, G, I, L, S, T, M, V, F, W, Y, P, or C; C453 replaced with D, E, H, K, R, A, G, I, L, S, T, M, V, N, Q, F, W, Y, or P; R454 replaced with D, E, A, G, I, L, S, T, M, V, N, Q, F, W, Y, P, or C; Q455 replaced with D, E, H, K, R, A, G, I, L, S, T, M, V, F, W, Y, P, or C; I456 replaced with D, E, H, K, R, N, Q, F, W, Y, P, or C; F457 replaced with D, E, H, K, R, N, Q, A, G, I, L, S, T, M, V, P, or C; G458 replaced with D, E, H, K, R, N, Q, F, W, Y, P, or C; P459 replaced with D, E, H, K, R, A, G, I, L, S, T, M, V, N, Q, F, W, Y, or C; D460 replaced with H, K, R, A, G, I, L, S, T, M, V, N, Q, F, W, Y, P, or C; F461 replaced with D, E, H, K, R, N, Q, A, G, I, L, S, T, M, V, P, or C; R462 replaced with D, E, A, G, I, L, S, T, M, V, N, Q, F, W, Y, P, or C; H463replaced with D, E, A, G, I, L, S, T, M, V, N, Q, F, W, Y, P, or C; C464 replaced with D, E, H, K, R, A, G, I, L, S, T, M, V, N, Q, F, W, Y, or P; P465 replaced with D, E, H, K, R, A, G, I, L, S, T, M, V, N, Q, F, W, Y, or C; N466 replaced with D, E, H, K, R, A, G, I, L, S, T, M, V, F, W, Y, P, or C; T467 replaced with D, E, H, K, R, N, Q, F, W, Y, P, or C; S468 replaced with D, E, H, K, R, N, Q, F, W, Y, P, or C; A469 replaced with D, E, H, K, R, N, Q, F, W, Y, P, or C; Q470 replaced with D, E, H, K, R, A, G, I, L, S, T, M, V, F, W, Y, P, or C; D471 replaced with H, K, R, A, G, I, L, S, T, M, V, N, Q, F, W, Y, P, or C; V472 replaced with D, E, H, K, R, N, Q, F, W, Y, P, or C; C473 replaced with D, E, H, K, R, A, G, I, L, S, T, M, V, N, Q, F, W, Y, or P; A474 replaced with D, E, H, K, R, N, Q, F, W, Y, P, or C; Q475 replaced with D, E, H, K, R, A, G, I, L, S, T, M, V, F, W, Y, P, or C; L476 replaced with D, E, H, K, R, N, Q, F, W, Y, P, or C; W477 replaced with D, E, H, K, R, N, Q, A, G, I, L, S, T, M, V, P, or C; C478 replaced with D, E, H, K, R, A, G, I, L, S, T, M, V, N, Q, F, W, Y, or P; H479 replaced with D, E, A, G, I, L, S, T, M, V, N, Q, F, W, Y, P, or C; T480 replaced with D, E, H, K, R, N, Q, F, W, Y, P, or C; D481 replaced with H, K, R, A, G, I, L, S, T, M, V, N, Q, F, W, Y, P, or C; G482 replaced with D, E, H, K, R, N, Q, F, W, Y, P, or C; A483 replaced with D, E, H, K, R, N, Q, F, W, Y, P, or C; E484 replaced with H, K, R, A, G, I, L, S, T, M, V, N, Q, F, W, Y, P, or C; P485 replaced with D, E, H, K, R, A, G, I, L, S, T, M, V, N, Q, F, W, Y, or C; I486 replaced with D, E, H, K, R, N, Q, F, W, Y, P, or C; C487 replaced with D, E, H, K, R, A, G, I, L, S, T, M, V, N, Q, F, W, Y, or P; H488 replaced with D, E, A, G, I, L, S, T, M, V, N, Q, F, W, Y, P, or C; T489 replaced with D, E, H, K, R, N, Q, F, W, Y, P, or C; K490 replaced with D, E, A, G, I, L, S, T, M, V, N, Q, F, W, Y, P, or C; N491 replaced with D, E, H, K, R, A, G, I, L, S, T, M, V, F, W, Y, P, or C; G492 replaced with D, E, H, K, R, N, Q, F, W, Y, P, or C; S493 replaced with D, E, H, K, R, N, Q, F, W, Y, P, or C; L494 replaced with D, E, H, K, R, N, Q, F, W, Y, P, or C; P495 replaced with D, E, H, K, R, A, G, I, L, S, T, M, V, N, Q, F, W, Y, or C; W496 replaced with D, E, H, K, R, N, Q, A, G, I, L, S, T, M, V, P, or C; A497 replaced with D, E, H, K, R, N, Q, F, W, Y, P, or C; D498 replaced with H, K, R, A, G, I, L, S, T, M, V, N, Q, F, W, Y, P, or C; G499 replaced with D, E, H, K, R, N, Q, F, W, Y, P, or C; T500 replaced with D, E, H, K, R, N, Q, F, W, Y, P, or C; P501 replaced with D, E, H, K, R, A, G, I, L, S, T, M, V, N, Q, F, W, Y, or C; C502 replaced with D, E, H, K, R, A, G, I, L, S, T, M, V, N, Q, F, W, Y, or P; G503 replaced with D, E, H, K, R, N, Q, F, W, Y, P, or C; P504 replaced with D, E, H, K, R, A, G, I, L, S, T, M, V, N, Q, F, W, Y, or C; G505 replaced with D, E, H, K, R, N, Q, F, W, Y, P, or C; H506 replaced with D, E, A, G, I, L, S, T, M, V, N, Q, F, W, Y, P, or C; L507 replaced with D, E, H, K, R, N, Q, F, W, Y, P, or C; C508 replaced with D, E, H, K, R, A, G, I, L, S, T, M, V, N, Q, F, W, Y, or P; S509 replaced with D, E, H, K, R, N, Q, F, W, Y, P, or C; E510 replaced with H, K, R, A, G, I, L, S, T, M, V, N, Q, F, W, Y, P, or C; G511 replaced with D, E, H, K, R, N, Q, F, W, Y, P, or C; S512 replaced with D, E, H, K, R, N, Q, F, W, Y, P, or C; C513 replaced with D, E, H, K, R, A, G, I, L, S, T, M, V, N, Q, F, W, Y, or P; L514 replaced with D, E, H, K, R, N, Q, F, W, Y, P, or C; P515 replaced with D, E, H, K, R, A, G, I, L, S, T, M, V, N, Q, F, W, Y, or C; E516 replaced with H, K, R, A, G, I, L, S, T, M, V, N, Q, F, W, Y, P, or C; E517 replaced with H, K, R, A, G, I, L, S, T, M, V, N, Q, F, W, Y, P, or C; E518 replaced with H, K, R, A, G, I, L, S, T, M, V, N, Q, F, W, Y, P, or C; V519 replaced with D, E, H, K, R, N, Q, F, W, Y, P, or C; E520 replaced with H, K, R, A, G, I, L, S, T, M, V, N, Q, F, W, Y, P, or C; R521 replaced with D, E, A, G, I, L, S, T, M, V, N, Q, F, W, Y, P, or C; P522 replaced with D, E, H, K, R, A, G, I, L, S, T, M, V, N, Q, F, W, Y, or C; K523 replaced with D, E, A, G, I, L, S, T, M, V, N, Q, F, W, Y, P, or C; P524 replaced with D, E, H, K, R, A, G, I, L, S, T, M, V, N, Q, F, W, Y, or C; V525 replaced with D, E, H, K, R, N, Q, F, W, Y, P, or C; V526 replaced with D, E, H, K, R, N, Q, F, W, Y, P, or C; D527 replaced with H, K, R, A, G, I, L, S, T, M, V, N, Q, F, W, Y, P, or C; G528 replaced with D, E, H, K, R, N, Q, F, W, Y, P, or C; G529 replaced with D, E, H, K, R, N, Q, F, W, Y, P, or C; W530 replaced with D, E, H, K, R, N, Q, A, G, I, L, S, T, M, V, P, or C; A531 replaced with D, E, H, K, R, N, Q, F, W, Y, P, or C; P532 replaced with D, E, H, K, R, A, G, I, L, S, T, M, V, N, Q, F, W, Y, or C; W533 replaced with D, E, H, K, R, N, Q, A, G, I, L, S, T, M, V, P, or C; G534 replaced with D, E, H, K, R, N, Q, F, W, Y, P, or C; P535 replaced with D, E, H, K, R, A, G, I, L, S, T, M, V, N, Q, F, W, Y, or C; W536 replaced with D, E, H, K, R, N, Q, A, G, I, L, S, T, M, V, P, or C; G537 replaced with D, E, H, K, R, N, Q, F, W, Y, P, or C; E538 replaced with H, K, R, A, G, I, L, S, T, M, V, N, Q, F, W, Y, P, or C; C539 replaced with D, E, H, K, R, A, G, I, L, S, T, M, V, N, Q, F, W, Y, or P; S540 replaced with D, E, H, K, R, N, Q, F, W, Y, P, or C; R541 replaced with D, E, A, G, I, L, S, T, M, V, N, Q, F, W, Y, P, or C; T542 replaced with D, E, H, K, R, N, Q, F, W, Y, P, or C; C543 replaced with D, E, H, K, R, A, G, I, L, S, T, M, V, N, Q, F, W, Y, or P; G544 replaced with D, E, H, K, R, N, Q, F, W, Y, P, or C; G545 replaced with D, E, H, K, R, N, Q, F, W, Y, P, or C; G546 replaced with D, E, H, K, R, N, Q, F, W, Y, P, or C; V547 replaced with D, E, H, K, R, N, Q, F, W, Y, P, or C; Q548 replaced with D, E, H, K, R, A, G, I, L, S, T, M, V, F, W, Y, P, or C; F549 replaced with D, E, H, K, R, N, Q, A, G, I, L, S, T, M, V, P, or C; S550 replaced with D, E, H, K, R, N, Q, F, W, Y, P, or C; H551 replaced with D, E, A, G, I, L, S, T, M, V, N, Q, F, W, Y, P, or C; R552 replaced with D, E, A, G, I, L, S, T, M, V, N, Q, F, W, Y, P, or C; E553 replaced with H, K, R, A, G, I, L, S, T, M, V, N, Q, F, W, Y, P, or C; C554 replaced with D, E, H, K, R, A, G, I, L, S, T, M, V, N, Q, F, W, Y, or P; K555 replaced with D, E, A, G, I, L, S, T, M, V, N, Q, F, W, Y, P, or C; D556 replaced with H, K, R, A, G, I, L, S, T, M, V, N, Q, F, W, Y, P, or C; P557 replaced with D, E, H, K, R, A, G, I, L, S, T, M, V, N, Q, F, W, Y, or C; E558 replaced with H, K, R, A, G, I, L, S, T, M, V, N, Q, F, W, Y, P, or C; P559 replaced with D, E, H, K, R, A, G, I, L, S, T, M, V, N, Q, F, W, Y, or C; Q560 replaced with D, E, H, K, R, A, G, I, L, S, T, M, V, F, W, Y, P, or C; N561 replaced with D, E, H, K, R, A, G, I, L, S, T, M, V, F, W, Y, P, or C; G562 replaced with D, E, H, K, R, N, Q, F, W, Y, P, or C; G563 replaced with D, E, H, K, R, N, Q, F, W, Y, P, or C; R564 replaced with D, E, A, G, I, L, S, T, M, V, N, Q, F, W, Y, P, or C; Y565 replaced with D, E, H, K, R, N, Q, A, G, I, L, S, T, M, V, P, or C; C566 replaced with D, E, H, K, R, A, G, I, L, S, T, M, V, N, Q, F, W, Y, or P; L567 replaced with D, E, H, K, R, N, Q, F, W, Y, P, or C; G568 replaced with D, E, H, K, R, N, Q, F, W, Y, P, or C; R569 replaced with D, E, A, G, I, L, S, T, M, V, N, Q, F, W, Y, P, or C, R570 replaced with D, E, A, G, I, L, S, T, M, V, N, Q, F, W, Y, P, or C; A571 replaced with D, E, H, K, R, N, Q, F, W, Y, P, or C; K572 replaced with D, E, A, G, I, L, S, T, M, V, N, Q, F, W, Y, P, or C; Y573 replaced with D, E, H, K, R, N, Q, A, G, I, L, S, T, M, V, P, or C; Q574 replaced with D, E, H, K, R, A, G, I, L, S, T, M, V, F, W, Y, P, or C; S575 replaced with D, E, H, K, R, N, Q, F, W, Y, P, or C; C576 replaced with D, E, H, K, R, A, G, I, L, S, T, M, V, N, Q, F, W, Y, or P; H577 replaced with D, E, A, G, I, L, S, T, M, V, N, Q, F, W, Y, P, or C; T578 replaced with D, E, H, K, R, N, Q, F, W, Y, P, or C; E579 replaced with H, K, R, A, G, I, L, S, T, M, V, N, Q, F, W, Y, P, or C; E580 replaced with H, K, R, A, G, I, L, S, T, M, V, N, Q, F, W, Y, P, or C; C581 replaced with D, E, H, K, R, A, G, I, L, S, T, M, V, N, Q, F, W, Y, or P; P582 replaced with D, E, H, K, R, A, G, I, L, S, T, M, V, N, Q, F, W, Y, or C; P583 replaced with D, E, H, K, R, A, G, I, L, S, T, M, V, N, Q, F, W, Y, or C; D584 replaced with H, K, R, A, G, I, L, S, T, M, V, N, Q, F, W, Y, P, or C; G585 replaced with D, E, H, K, R, N, Q, F, W, Y, P, or C; K586 replaced with D, E, A, G, I, L, S, T, M, V, N, Q, F, W, Y, P, or C; S587 replaced with D, E, H, K, R, N, Q, F, W, Y, P, or C; F588 replaced with D, E, H, K, R, N, Q, A, G, I, L, S, T, M, V, P, or C; R589 replaced with D, E, A, G, I, L, S, T, M, V, N, Q, F, W, Y, P, or C; E590 replaced with H, K, R, A, G, I, L, S, T, M, V, N, Q, F, W, Y, P, or C; Q591 replaced with D, E, H, K, R, A, G, I, L, S, T, M, V, F, W, Y, P, or C; Q592 replaced with D, E, H, K, R, A, G, I, L, S, T, M, V, F, W, Y, P, or C; C593 replaced with D, E, H, K, R, A, G, I, L, S, T, M, V, N, Q, F, W, Y, or P; E594 replaced with H, K, R, A, G, I, L, S, T, M, V, N, Q, F, W, Y, P, or C; K595 replaced with D, E, A, G, I, L, S, T, M, V, N, Q, F, W, Y, P, or C; Y596 replaced with D, E, H, K, R, N, Q, A, G, I, L, S, T, M, V, P, or C; N597 replaced with D, E, H, K, R, A, G, I, L, S, T, M, V, F, W, Y, P, or C; A598 replaced with D, E, H, K, R, N, Q, F, W, Y, P, or C; Y599 replaced with D, E, H, K, R, N, Q, A, G, I, L, S, T, M, V, P, or C; N600 replaced with D, E, H, K, R, A, G, I, L, S, T, M, V, F, W, Y, P, or C; Y601 replaced with D, E, H, K, R, N, Q, A, G, I, L, S, T, M, V, P, or C; T602 replaced with D, E, H, K, R, N, Q, F, W, Y, P, or C; D603 replaced with H, K, R, A, G, I, L, S, T, M, V, N, Q, F, W, Y, P, or C; M604 replaced with D, E, H, K, R, N, Q, F, W, Y, P, or C; D605 replaced with H, K, R, A, G, I, L, S, T, M, V, N, Q, F, W, Y, P, or C; G606 replaced with D, E, H, K, R, N, Q, F, W, Y, P, or C; N607 replaced with D, E, H, K, R, A, G, I, L, S, T, M, V, F, W, Y, P, or C; L608 replaced with D, E, H, K, R, N, Q, F, W, Y, P, or C; L609 replaced with D, E, H, K, R, N, Q, F, W, Y, P, or C; Q610 replaced with D, E, H, K, R, A, G, I, L, S, T, M, V, F, W, Y, P, or C; W611 replaced with D, E, H, K, R, N, Q, A, G, I, L, S, T, M, V, P, or C; V612 replaced with D, E, H, K, R, N, Q, F, W, Y, P, or C; P613 replaced with D, E, H, K, R, A, G, I, L, S, T, M, V, N, Q, F, W, Y, or C; K614 replaced with D, E, A, G, I, L, S, T, M, V, N, Q, F, W, Y, P, or C; Y615 replaced with D, E, H, K, R, N, Q, A, G, I, L, S, T, M, V, P, or C; A616 replaced with D, E, H, K, R, N, Q, F, W, Y, P, or C; G617 replaced with D, E, H, K, R, N, Q, F, W, Y, P, or C; V618 replaced with D, E, H, K, R, N, Q, F, W, Y, P, or C; S619 replaced with D, E, H, K, R, N, Q, F, W, Y, P, or C; P620 replaced with D, E, H, K, R, A, G, I, L, S, T, M, V, N, Q, F, W, Y, or C; R621 replaced with D, E, A, G, I, L, S, T, M, V, N, Q, F, W, Y, P, or C; D622 replaced with H, K, R, A, G, I, L, S, T, M, V, N, Q, F, W, Y, P, or C; R623 replaced with D, E, A, G, I, L, S, T, M, V, N, Q, F, W, Y, P, or C; C624 replaced with D, E, H, K, R, A, G, I, L, S, T, M, V, N, Q, F, W, Y, or P; K625 replaced with D, E, A, G, I, L, S, T, M, V, N, Q, F, W, Y, P, or C; L626 replaced with D, E, H, K, R, N, Q, F, W, Y, P, or C; F627 replaced with D, E, H, K, R, N, Q, A, G, I, L, S, T, M, V, P, or C; C628 replaced with D, E, H, K, R, A, G, I, L, S, T, M, V, N, Q, F, W, Y, or P; R629 replaced with D, E, A, G, I, L, S, T, M, V, N, Q, F, W, Y, P, or C; A630 replaced with D, E, H, K, R, N, Q, F, W, Y, P, or C; R631 replaced with D, E, A, G, I, L, S, T, M, V, N, Q, F, W, Y, P, or C; G632 replaced with D, E, H, K, R, N, Q, F, W, Y, P, or C; R633 replaced with D, E, A, G, I, L, S, T, M, V, N, Q, F, W, Y, P, or C; S634 replaced with D, E, H, K, R, N, Q, F, W, Y, P, or C; E635 replaced with H, K, R, A, G, I, L, S, T, M, V, N, Q, F, W, Y, P, or C; F636 replaced with D, E, H, K, R, N, Q, A, G, I, L, S, T, M, V, P, or C; K637 replaced with D, E, A, G, I, L, S, T, M, V, N, Q, F, W, Y, P, or C; V638 replaced with D, E, H, K, R, N, Q, F, W, Y, P, or C; F639 replaced with D, E, H, K, R, N, Q, A, G, I, L, S, T, M, V, P, or C; E640 replaced with H, K, R, A, G, I, L, S, T, M, V, N, Q, F, W, Y, P, or C; A641 replaced with D, E, H, K, R, N, Q, F, W, Y, P, or C; K642 replaced with D, E, A, G, I, L, S, T, M, V, N, Q, F, W, Y, P, or C; V643 replaced with D, E, H, K, R, N, Q, F, W, Y, P, or C; I644 replaced with D, E, H, K, R, N, Q, F, W, Y, P, or C; D645 replaced with H, K, R, A, G, I, L, S, T, M, V, N, Q, F, W, Y, P, or C; G646 replaced with D, E, H, K, R, N, Q, F, W, Y, P, or C; T647 replaced with D, E, H, K, R, N, Q, F, W, Y, P, or C; L648 replaced with D, E, H, K, R, N, Q, F, W, Y, P, or C; C649 replaced with D, E, H, K, R, A, G, I, L, S, T, M, V, N, Q, F, W, Y, or P; G650 replaced with D, E, H, K, R, N, Q, F, W, Y, P, or C; P651 replaced with D, E, H, K, R, A, G, I, L, S, T, M, V, N, Q, F, W, Y, or C; E652 replaced with H, K, R, A, G, I, L, S, T, M, V, N, Q, F, W, Y, P, or C; T653 replaced with D, E, H, K, R, N, Q, F, W, Y, P, or C; L654 replaced with D, E, H, K, R, N, Q, F, W, Y, P, or C; A655 replaced with D, E, H, K, R, N, Q, F, W, Y, P, or C; I656 replaced with D, E, H, K, R, N, Q, F, W, Y, P, or C; C657 replaced with D, E, H, K, R, A, G, I, L, S, T, M, V, N, Q, F, W, Y, or P; V658 replaced with D, E, H, K, R, N, Q, F, W, Y, P, or C; R659 replaced with D, E, A, G, I, L, S, T, M, V, N, Q, F, W, Y, P, or C; G660 replaced with D, E, H, K, R, N, Q, F, W, Y, P, or C; Q661 replaced with D, E, H, K, R, A, G, I, L, S, T, M, V, F, W, Y, P, or C; C662 replaced with D, E, H, K, R, A, G, I, L, S, T, M, V, N, Q, F, W, Y, or P; V663 replaced with D, E, H, K, R, N, Q, F, W, Y, P, or C; K664 replaced with D, E, A, G, I, L, S, T, M, V, N, Q, F, W, Y, P, or C; A665 replaced with D, E, H, K, R, N, Q, F, W, Y, P, or C; G666 replaced with D, E, H, K, R, N, Q, F, W, Y, P, or C; C667 replaced with D, E, H, K, R, A, G, I, L, S, T, M, V, N, Q, F, W, Y, or P; D668 replaced with H, K, R, A, G, I, L, S, T, M, V, N, Q, F, W, Y, P, or C; H669 replaced with D, E, A, G, I, L, S, T, M, V, N, Q, F, W, Y, P, or C; V670 replaced with D, E, H, K, R, N, Q, F, W, Y, P, or C; V671 replaced with D, E, H, K, R, N, Q, F, W, Y, P, or C; D672 replaced with H, K, R, A, G, I, L, S, T, M, V, N, Q, F, W, Y, P, or C; S673 replaced with D, E, H, K, R, N, Q, F, W, Y, P, or C; P674 replaced with D, E, H, K, R, A, G, I, L, S, T, M, V, N, Q, F, W, Y, or C; R675 replaced with D, E, A, G, I, L, S, T, M, V, N, Q, F, W, Y, P, or C; K676 replaced with D, E, A, G, I, L, S, T, M, V, N, Q, F, W, Y, P, or C; L677 replaced with D, E, H, K, R, N, Q, F, W, Y, P, or C; D678 replaced with H, K, R, A, G, I, L, S, T, M, V, N, Q, F, W, Y, P, or C; K679 replaced with D, E, A, G, I, L, S, T, M, V, N, Q, F, W, Y, P, or C; C680 replaced with D, E, H, K, R, A, G, I, L, S, T, M, V, N, Q, F, W, Y, or P; G681 replaced with D, E, H, K, R, N, Q, F, W, Y, P, or C; V682 replaced with D, E, H, K, R, N, Q, F, W, Y, P, or C; C683 replaced with D, E, H, K, R, A, G, I, L, S, T, M, V, N, Q, F, W, Y, or P; G684 replaced with D, E, H, K, R, N, Q, F, W, Y, P, or C; G685 replaced with D, E, H, K, R, N, Q, F, W, Y, P, or C; K686 replaced with D, E, A, G, I, L, S, T, M, V, N, Q, F, W, Y, P, or C; G687 replaced with D, E, H, K, R, N, Q, F, W, Y, P, or C; N688 replaced with D, E, H, K, R, A, G, I, L, S, T, M, V, F, W, Y, P, or C; S689 replaced with D, E, H, K, R, N, Q, F, W, Y, P, or C; C690 replaced with D, E, H, K, R, A, G, I, L, S, T, M, V, N, Q, F, W, Y, or P; R691 replaced with D, E, A, G, I, L, S, T, M, V, N, Q, F, W, Y, P, or C; K692 replaced with D, E, A, G, I, L, S, T, M, V, N, Q, F, W, Y, P, or C; V693 replaced with D, E, H, K, R, N, Q, F, W, Y, P, or C; S694 replaced with D, E, H, K, R, N, Q, F, W, Y, P, or C; G695 replaced with D, E, H, K, R, N, Q, F, W, Y, P, or C; S696 replaced with D, E, H, K, R, N, Q, F, W, Y, P, or C; L697 replaced with D, E, H, K, R, N, Q, F, W, Y, P, or C; T698replaced with D, E, H, K, R, N, Q, F, W, Y, P, or C; P699 replaced with D, E, H, K, R, A, G, I, L, S, T, M, V, N, Q, F, W, Y, or C; T700 replaced with D, E, H, K, R, N, Q, F, W, Y, P, or C; N701 replaced with D, E, H, K, R, A, G, I, L, S, T, M, V, F, W, Y, P, or C; Y702 replaced with D, E, H, K, R, N, Q, A, G, I, L, S, T, M, V, P, or C; G703 replaced with D, E, H, K, R, N, Q, F, W, Y, P, or C; Y704 replaced with D, E, H, K, R, N, Q, A, G, I, L, S, T, M, V, P, or C; N705 replaced with D, E, H, K, R, A, G, I, L, S, T, M, V, F, W, Y, P, or C; D706 replaced with H, K, R, A, G, I, L, S, T, M, V, N, Q, F, W, Y, P, or C; I707 replaced with D, E, H, K, R, N, Q, F, W, Y, P, or C; V708 replaced with D, E, H, K, R, N, Q, F, W, Y, P, or C; T709 replaced with D, E, H, K, R, N, Q, F, W, Y, P, or C; I710 replaced with D, E, H, K, R, N, Q, F, W, Y, P, or C; P711 replaced with D, E, H, K, R, A, G, I, L, S, T, M, V, N, Q, F, W, Y, or C; A712 replaced with D, E, H, K, R, N, Q, F, W, Y, P, or C; G713 replaced with D, E, H, K, R, N, Q, F, W, Y, P, or C; A714 replaced with D, E, H, K, R, N, Q, F, W, Y, P, or C; T715 replaced with D, E, H, K, R, N, Q, F, W, Y, P, or C; N716 replaced with D, E, H, K, R, A, G, I, L, S, T, M, V, F, W, Y, P, or C; I717 replaced with D, E, H, K, R, N, Q, F, W, Y, P, or C; D718 replaced with H, K, R, A, G, I, L, S, T, M, V, N, Q, F, W, Y, P, or C; V719 replaced with D, E, H, K, R, N, Q, F, W, Y, P, or C; K720 replaced with D, E, A, G, I, L, S, T, M, V, N, Q, F, W, Y, P, or C; Q721 replaced with D, E, H, K, R, A, G, I, L, S, T, M, V, F, W, Y, P, or C; R722 replaced with D, E, A, G, I, L, S, T, M, V, N, Q, F, W, Y, P, or C; S723 replaced with D, E, H, K, R, N, Q, F, W, Y, P, or C; H724 replaced with D, E, A, G, I, L, S, T, M, V, N, Q, F, W, Y, P, or C; P725 replaced with D, E, H, K, R, A, G, I, L, S, T, M, V, N, Q, F, W, Y, or C; G726 replaced with D, E, H, K, R, N, Q, F, W, Y, P, or C; V727 replaced with D, E, H, K, R, N, Q, F, W, Y, P, or C; Q728 replaced with D, E, H, K, R, A, G, I, L, S, T, M, V, F, W, Y, P, or C; N729 replaced with D, E, H, K, R, A, G, I, L, S, T, M, V, F, W, Y, P, or C; D730 replaced with H, K, R, A, G, I, L, S, T, M, V, N, Q, F, W, Y, P, or C; G731 replaced with D, E, H, K, R, N, Q, F, W, Y, P, or C; N732 replaced with D, E, H, K, R, A, G, I, L, S, T, M, V, F, W, Y, P, or C; Y733 replaced with D, E, H, K, R, N, Q, A, G, I, L, S, T, M, V, P, or C; L734 replaced with D, E, H, K, R, N, Q, F, W, Y, P, or C; A735 replaced with D, E, H, K, R, N, Q, F, W, Y, P, or C; L736 replaced with D, E, H, K, R, N, Q, F, W, Y, P, or C; K737 replaced with D, E, A, G I, L, S, T, M, V, N, Q, F, W, Y, P, or C; T738 replaced with D, E, H, K, R, N, Q, F, W, Y, P, or C; A739 replaced with D, E, H, K, R, N, Q, F, W, Y, P, or C; D740 replaced with H, K, R, A, G, I, L, S, T, M, V, N, Q, F, W, Y, P, or C; G741 replaced with D, E, H, K, R, N, Q, F, W, Y, P, or C; Q742 replaced with D, E, H, K, R, A, G, I, L, S, T, M, V, F, W, Y, P, or C; Y743 replaced with D, E, H, K, R, N, Q, A, G, I, L, S, T, M, V, P, or C; L744 replaced with D, E, H, K, R, N, Q, F, W, Y, P, or C; L745 replaced with D, E, H, K, R, N, Q, F, W, Y, P, or C; N746 replaced with D, E, H, K, R, A, G, I, L, S, T, M, V, F, W, Y, P, or C; G747 replaced with D, E, H, K, R, N, Q, F, W, Y, P, or C; N748 replaced with D, E, H, K, R, A, G, I, L, S, T, M, V, F, W, Y, P, or C; L749 replaced with D, E, H, K, R, N, Q, F, W, Y, P, or C; A750 replaced with D, E, H, K, R, N, Q, F, W, Y, P, or C; I751 replaced with D, E, H, K, R, N, Q, F, W, Y, P, or C; S752 replaced with D, E, H, K, R, N, Q, F, W, Y, P, or C; A753 replaced with D, E, H, K, R, N, Q, F, W, Y, P, or C; I754 replaced with D, E, H, K, R, N, Q, F, W, Y, P, or C; E755 replaced with H, K, R, A, G, I, L, S, T, M, V, N, Q, F, W, Y, P, or C; Q756 replaced with D, E, H, K, R, A, G, I, L, S, T, M, V, F, W, Y, P, or C; D757 replaced with H, K, R, A, G, I, L, S, T, M, V, N, Q, F, W, Y, P, or C; I758 replaced with D, E, H, K, R, N, Q, F, W, Y, P, or C; L759 replaced with D, E, H, K, R, N, Q, F, W, Y, P, or C; V760 replaced with D, E, H, K, R, N, Q, F, W, Y, P, or C; K761 replaced with D, E, A, G, I, L, S, T, M, V, N, Q, F, W, Y, P, or C; G762 replaced with D, E, H, K, R, N, Q, F, W, Y, P, or C; T763 replaced with D, E, H, K, R, N, Q, F, W, Y, P, or C; I764 replaced with D, E, H, K, R, N, Q, F, W, Y, P, or C; L765 replaced with D, E, H, K, R, N, Q, F, W, Y, P, or C; K766 replaced with D, E, A, G, I, L, S, T, M, V, N, Q, F, W, Y, P, or C; Y767 replaced with D, E, H, K, R, N, Q, A, G, I, L, S, T, M, V, P, or C; S768 replaced with D, E, H, K, R, N, Q, F, W, Y, P, or C; G769 replaced with D, E, H, K, R, N, Q, F, W, Y, P, or C; S770 replaced with D, E, H, K, R, N, Q, F, W, Y, P, or C; I771 replaced with D, E, H, K, R, N, Q, F, W, Y, P, or C; A772 replaced with D, E, H, K, R, N, Q, F, W, Y, P, or C; T773 replaced with D, E, H, K, R, N, Q, F, W, Y, P, or C; L774 replaced with D, E, H, K, R, N, Q, F, W, Y, P, or C; E775 replaced with H, K, R, A, G, I, L, S, T, M, V, N, Q, F, W, Y, P, or C; R776 replaced with D, E, A, G, I, L, S, T, M, V, N, Q, F, W, Y, P, or C; L777replaced with D, E, H, K, R, N, Q, F, W, Y, P, or C; Q778 replaced with D, E, H, K, R, A, G, I, L, S, T, M, V, F, W, Y, P, or C; S779 replaced with D, E, H, K, R, N, Q, F, W, Y, P, or C; F780 replaced with D, E, H, K, R, N, Q, A, G, I, L, S, T, M, V, P, or C; R781 replaced with D, E, A, G, I, L, S, T, M, V, N, Q, F, W, Y, P, or C; P782 replaced with D, E, H, K, R, A, G, I, L, S, T, M, V, N, Q, F, W, Y, or C; L783 replaced with D, E, H, K, R, N, Q, F, W, Y, P, or C; P784 replaced with D, E, H, K, R, A, G, I, L, S, T, M, V, N, Q, F, W, Y, or C; E785 replaced with H, K, R, A, G, I, L, S, T, M, V, N, Q, F, W, Y, P, or C; P786 replaced with D, E, H, K, R, A, G, I, L, S, T, M, V, N, Q, F, W, Y, or C; L787 replaced with D, E, H, K, R, N, Q, F, W, Y, P, or C; T788 replaced with D, E, H, K, R, N, Q, F, W, Y, P, or C; V789 replaced with D, E, H, K, R, N, Q, F, W, Y, P, or C; Q790 replaced with D, E, H, K, R, A, G, I, L, S, T, M, V, F, W, Y, P, or C; L791 replaced with D, E, H, K, R, N, Q, F, W, Y, P, or C; L792 replaced with D, E, H, K, R, N, Q, F, W, Y, P, or C; T793 replaced with D, E, H, K, R, N, Q, F, W, Y, P, or C; V794 replaced with D, E, H, K, R, N, Q, F, W, Y, P, or C; P795 replaced with D, E, H, K, R, A, G, I, L, S, T, M, V, N, Q, F, W, Y, or C; G796 replaced with D, E, H, K, R, N, Q, F, W, Y, P, or C; E797 replaced with H, K, R, A, G, I, L, S, T, M, V, N, Q, F, W, Y, P, or C; V798 replaced with D, E, H, K, R, N, Q, F, W, Y, P, or C; F799 replaced with D, E, H, K, R, N, Q, A, G, I, L, S, T, M, V, P, or C; P800 replaced with D, E, H, K, R, A, G, I, L, S, T, M, V, N, Q, F, W, Y, or C; P801 replaced with D, E, H, K, R, A, G, I, L, S, T, M, V, N, Q, F, W, Y, or C; K802 replaced with D, E, A, G, I, L, S, T, M, V, N, Q, F, W, Y, P, or C; V803 replaced with D, E, H, K, R, N, Q, F, W, Y, P, or C; K804 replaced with D, E, A, G, I, L, S, T, M, V, N, Q, F, W, Y, P, or C; Y805 replaced with D, E, H, K, R, N, Q, A, G, I, L, S, T, M, V, P, or C; T806 replaced with D, E, H, K, R, N, Q, F, W, Y, P, or C; F807 replaced with D, E, H, K, R, N, Q, A, G, I, L, S, T, M, V, P, or C; F808 replaced with D, E, H, K, R, N, Q, A, G, I, L, S, T, M, V, P, or C; V809 replaced with D, E, H, K, R, N, Q, F, W, Y, P, or C; P810replaced with D, E, H, K, R, A, G, I, L, S, T, M, V, N, Q, F, W, Y, or C; N811 replaced with D, E, H, K, R, A, G, I, L, S, T, M, V, F, W, Y, P, or C; D812 replaced with H, K, R, A, G, I, L, S, T, M, V, N, Q, F, W, Y, P, or C; V813 replaced with D, E, H, K, R, N, Q, F, W, Y, P, or C; D814 replaced with H, K, R, A, G, I, L, S, T, M, V, N, Q, F, W, Y, P, or C; F815 replaced with D, E, H, K, R, N, Q, A, G, I, L, S, T, M, V, P, or C; S816 replaced with D, E, H, K, R, N, Q, F, W, Y, P, or C; M817 replaced with D, E, H, K, R, N, Q, F, W, Y, P, or C; Q818 replaced with D, E, H, K, R, A, G, I, L, S, T, M, V, F, W, Y, P, or C; S819 replaced with D, E, H, K, R, N, Q, F, W, Y, P, or C; S820 replaced with D, E, H, K, R, N, Q, F, W, Y, P, or C; K821 replaced with D, E, A, G, I, L, S, T, M, V, N, Q, F, W, Y, P, or C; E822 replaced with H, K, R, A, G, I, L, S, T, M, V, N, Q, F, W, Y, P, or C; R823 replaced with D, E, A, G, I, L, S, T, M, V, N, Q, F, W, Y, P, or C; A824 replaced with D, E, H, K, R, N, Q, F, W, Y, P, or C; T825 replaced with D, E, H, K, R, N, Q, F, W, Y, P, or C; T826 replaced with D, E, H, K, R, N, Q, F, W, Y, P, or C; N827 replaced with D, E, H, K, R, A, G, I, L, S, T, M, V, F, W, Y, P, or C; I828 replaced with D, E, H, K, R, N, Q, F, W, Y, P, or C; I829 replaced with D, E, H, K, R, N, Q, F, W, Y, P, or C; Q830 replaced with D, E, H, K, R, A, G, I, L, S, T, M, V, F, W, Y, P, or C; P831 replaced with D, E, H, K, R, A, G, I, L, S, T, M, V, N, Q, F, W, Y, or C; L832 replaced with D, E, H, K, R, N, Q, F, W, Y, P, or C; L833 replaced with D, E, H, K, R, N, Q, F, W, Y, P, or C; H834 replaced with D, E, A, G, I, L, S, T, M, V, N, Q, F, W, Y, P, or C; A835 replaced with D, E, H, K, R, N, Q, F, W, Y, P, or C; Q836 replaced with D, E, H, K, R, A, G, I, L, S, T, M, V, F, W, Y, P, or C; W837 replaced with D, E, H, K, R, N, Q, A, G, I, L, S, T, M, V, P, or C; V838 replaced with D, E, H, K, R, N, Q, F, W, Y, P, or C; L839 replaced with D, E, H, K, R, N, Q, F, W, Y, P, or C; G840 replaced with D, E, H, K, R, N, Q, F, W, Y, P, or C; D841 replaced with H, K, R, A, G, I, L, S, T, M, V, N, Q, F, W, Y, P, or C; W842 replaced with D, E, H, K, R, N, Q, A, G, I, L, S, T, M, V, P, or C; S843 replaced with D, E, H, K, R, N, Q, F, W, Y, P, or C; E844 replaced with H, K, R, A, G, I, L, S, T, M, V, N, Q, F, W, Y, P, or C; C845 replaced with D, E, H, K, R, A, G, I, L, S, T, M, V, N, Q, F, W, Y, or P; S846 replaced with D, E, H, K, R, N, Q, F, W, Y, P, or C; S847 replaced with D, E, H, K, R, N, Q, F, W, Y, P, or C; T848 replaced with D, E, H, K, R, N, Q, F, W, Y, P, or C; C849 replaced with D, E, H, K, R, A, G, I, L, S, T, M, V, N, Q, F, W, Y, or P; G850 replaced with D, E, H, K, R, N, Q, F, W, Y, P, or C; A851 replaced with D, E, H, K, R, N, Q, F, W, Y, P, or C; G852 replaced with D, E, H, K, R, N, Q, F, W, Y, P, or C; W853 replaced with D, E, H, K, R, N, Q, A, G, I, L, S, T, M, V, P, or C; Q854 replaced with D, E, H, K, R, A, G, I, L, S, T, M, V, F, W, Y, P, or C; R855 replaced with D, E, A, G, I, L, S, T, M, V, N, Q, F, W, Y, P, or C; R856 replaced with D, E, A, G, I, L, S, T, M, V, N, Q, F, W, Y, P, or C; T857 replaced with D, E, H, K, R, N, Q, F, W, Y, P, or C; V858 replaced with D, E, H, K, R, N, Q, F, W, Y, P, or C; E859 replaced with H, K, R, A, G, I, L, S, T, M, V, N, Q, F, W, Y, P, or C; C860 replaced with D, E, H, K, R, A, G, I, L, S, T, M, V, N, Q, F, W, Y, or P; R861 replaced with D, E, A, G, I, L, S, T, M, V, N, Q, F, W, Y, P, or C; D862 replaced with H, K, R, A, G, I, L, S, T, M, V, N, Q, F, W, Y, P, or C; P863 replaced with D, E, H, K, R, A, G, I, L, S, T, M, V, N, Q, F, W, Y, or C; S864 replaced with D, E, H, K, R, N, Q, F, W, Y, P, or C; G865 replaced with D, E, H, K, R, N, Q, F, W, Y, P, or C; Q866 replaced with D, E, H, K, R, A, G, I, L, S, T, M, V, F, W, Y, P, or C; A867 replaced with D, E, H, K, R, N, Q, F, W, Y, P, or C; S868 replaced with D, E, H, K, R, N, Q, F, W, Y, P, or C; A869 replaced with D, E, H, K, R, N, Q, F, W, Y, P, or C; T870 replaced with D, E, H, K, R, N, Q, F, W, Y, P, or C; C871 replaced with D, E, H, K, R, A, G, I, L, S, T, M, V, N, Q, F, W, Y, or P; N872 replaced with D, E, H, K, R, A, G, I, L, S, T, M, V, F, W, Y, P, or C; K873 replaced with D, E, A, G, I, L, S, T, M, V, N, Q, F, W, Y, P, or C; A874 replaced with D, E, H, K, R, N, Q, F, W, Y, P, or C; L875 replaced with D, E, H, K, R, N, Q, F, W, Y, P, or C; K876 replaced with D, E, A, G, I, L, S, T, M, V, N, Q, F, W, Y, P, or C; P877 replaced with D, E, H, K, R, A, G, I, L, S, T, M, V, N, Q, F, W, Y, or C; E878 replaced with H, K, R, A, G, I, L, S, T, M, V, N, Q, F, W, Y, P, or C; D879 replaced with H, K, R, A, G, I, L, S, T, M, V, N, Q, F, W, Y, P, or C; A880 replaced with D, E; H, K, R, N, Q, F, W, Y, P, or C; K881 replaced with D, E, A, G, I, L, S, T, M, V, N, Q, F, W, Y, P, or C; P882 replaced with D, E, H, K, R, A, G, I, L, S, T, M, V, N, Q, F, W, Y, or C; C883 replaced with D, E, H, K, R, A, G, I, L, S, T, M, V, N, Q, F, W, Y, or P; E884replaced with H, K, R, A, G, I, L, S, T, M, V, N, Q, F, W, Y, P, or C; S885 replaced with D, E, H, K, R, N, Q, F, W, Y, P, or C; Q886 replaced with D, E, H, K, R, A, G, I, L, S, T, M, V, F, W, Y, P, or C; L887 replaced with D, E, H, K, R, N, Q, F, W, Y, P, or C; C888 replaced with D, E, H, K, R, A, G, I, L, S, T, M, V, N, Q, F, W, Y, or P; P889 replaced with D, E, H, K, R, A, G, I, L, S, T, M, V, N, Q, F, W, Y, or C; L890 replaced with D, E, H, K, R, N, Q, F, W, Y, P, or C.

METH1 or METH2 polypeptides may contain 50, 40, 30, 10, 20, 19, 18, 17, 16, 15, 14, 13, 12, 11, 10, 9, 8, 7, 6, 5, 4, 3, 2 or 1 conservative or non-conservative amino acid substitutions. Additionally, METH1 or METH2 polypeptides may contain both conservative or non-conservative substitutions, in any combination. A METH1 or METH2 polypeptide may contain 50, 40, 30, 10, 20, 19, 18, 17, 16, 15, 14, 13, 12, 11, 10, 9, 8, 7, 6, 5, 4, 3, 2 or 1 conservative amino acids substitutions, and 50, 40, 30, 10, 20, 19, 18, 17, 16, 15, 14, 13, 12, 11, 10, 9, 8, 7, 6, 5, 4, 3, 2 or 1 non-conservative amino acid substitutions in the same polypeptide. For example, a particular polypeptide may contain 10 conservative amino acid substitutions and 10 non-conservative amino acid substitutions. Polynucleotides encoding such METH1 or METH2 polypeptides with substitutions are also encompassed within the present invention.

The substitutions may be made in full-length METH1 or METH2, mature METH1 or METH2, and any other METH1 or METH2 variant disclosed herein, including METH1 or METH2 polypeptides with N- and/or C-terminal amino acid deletions; METH1 or METH2 polypeptides which lack one or more domains; or hybrid METH1/METH2 molecules.

Amino acids in the METH1 and METH2 proteins of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as in vitro or in vivo inhibition of angiogenesis. Sites that are critical for inhibition of angiogenesis can also be determined by structural analysis such as crystallization, nuclear magnetic resonance or photoaffinity labeling (Smith et al., J. Mol. Biol. 224:899-904 (1992) and de Vos et al., Science 255:306-312 (1992)).

Particularly preferred are polypeptides with amino acid substitutions at the boundaries of each domain (for example, at the boundary of the metalloprotease domain). Amino acid substitutions at these boundaries may be made to change the activity of the protein, for example, to prevent cleavage. Amino acid substitutions may also be made which do not affect the activity of the protein. For example, the following amino acids may be replaced in METH1, with the following amino acids: L-19 may be replaced with A, C, D, E, F, G, H, I, K, M, N, P, Q, R, S, T, V, W or Y; L-20 may be replaced with A, C, D, E, F, G, H, I, K, M, N, P, Q, R, S, T, V, W or Y; L-21 may be replaced with A, C, D, E, F, G, H, I, K, M, N, P, Q, R, S, T, V, W or Y; L-22 may be replaced with A, C, D, E, F, G, H, I, K, M, N, P, Q, R, S, T, V, W or Y; A-23 may be replaced with may be replaced with C, D, E, F, G, H, I, K, L, M, N, P, Q, R, S, T, V, W or Y; A-24 may be replaced with C, D, E, F, G, H, I, K, L, M, N, P, Q, R, S, T, V, W or Y; A-25 may be replaced with C, D, E, F, G, H, I, K, L, M, N, P, Q, R, S, T, V, W or Y; L-26 may be replaced with A, C, D, E, F, G, H, I, K, M, N, P, Q, R, S, T, V, W or Y; L-27 may be replaced with A, C, D, E, F, G, H, I, K, M, N, P, Q, R, S, T, V, W or Y; A-28 may be replaced with C, D, E, F, G, H, I, K, L, M, N, P, Q, R, S, T, V, W or Y; V-29 may be replaced with A, C, D, E, F, G, H, I, K, L, M, N, P, Q, R, S, T, W or Y; S-30 may be replaced with A, C, D, E, F, G, H, I, K, L, M, N, P, Q, R, T, V, W or Y; D-31 may be replaced with A, C, E, F, G, H, I, K, L, M, N, P, Q, R, S, T, V, W or Y; A-32 may be replaced with C, D, E, F, G, H, I, K, L, M, N, P, Q, R, S, T, V, W or Y; L-33 may be replaced with A, C, D, E, F, G, H, I, K, M, N, P, Q, R, S, T, V, W or Y; G-34 may be replaced with A, C, D, E, F, H, I, K, L, M, N, P, Q, R, S, T, V, W or Y; R-35 may be replaced with A, C, D, E, F, G, H, I, K, L, M, N, P, Q, S, T, V, W or Y; P-36 may be replaced with A, C, D, E, F, G, H, I, K, L, M, N, Q, R, S, T, V, W or Y; S-37 may be replaced with A, C, D, E, F, G, H, I, K, L, M, N, P, Q, R, T, V, W or Y; E-38 may be replaced with A, C, D, F, G, H, I, K, L, M, N, P, Q, R, S, T, V, W or Y; E-39 may be replaced with A, C, D, E, F, G, H, I, K, L, M, N, P, Q, R, S, T, V, W or Y; P-225 may be replaced with A, C, D, E, F, G, H, I, K, L, M, N, Q, R, S, T, V, W or Y; T-226 may be replaced with A, C, D, E, F, G, H, I, K, L, M, N, P, Q, R, S, V, W or Y; G-227 may be replaced with A, C, D, E, F, H, I, K, L, M, N, P, Q, R, S, T, V, W or Y; T-228 may be replaced with A, C, D, E, F, G, H, I, K, L, M, N, P, Q, R, S, V, W or Y; G-229 may be replaced with A, C, D, E, F, H, I, K, L, M, N, P, Q, R, S, T, V, W or Y; S-230 may be replaced with A, C, D, E, F, G, I, H, I, K, L, M, N, P, Q, R, T, V, W or Y; I-231 may be replaced with A, C, D, E, F, G, H, K, L, M, N, P, Q, R, S, T, V, W or Y; R-232 may be replaced with A, C, D, E, F, G, H, I, K, L, M, N, P, Q, S, T, V, W or Y; K-233 may be replaced with A, C, D, E, F, G, H, I, L, M, N, P, Q, R, S, T, V, W or Y; K-234 may be replaced with A, C, D, E, F, G, H, I, L, M, N, P, Q, R, S, T, V, W or Y; R-235 may be replaced with A, C, D, E, F, G, H, I, K, L, M, N, P, Q, S, T, V, W or Y; F-236 may be replaced with A, C, D, E, G, H, I, K, L, M, N, P, Q, R, S, T, V, W or Y; V-237 may be replaced with A, C, D, E, F, G, H, I, K, L, M, N, P, Q, R, S, T, W or Y; S-238 may be replaced with A, C, D, E, F, G, H, I, K, L, M, N, P, Q, R, T, V, W or Y; S-239 may be replaced with A, C, D, E, F, G, H, I, K, L, M, N, P, Q, R, T, V, W or Y; H-240 may be replaced with A, C, D, E, F, G, I, K, L, M, N, P, Q, R, S, T, V, W or Y; R-241 may be replaced with A, C, D, E, F, G, H, I, K, L, M, N, P, Q, S, T, V, W or Y; Y-242 may be replaced with A, C, D, E, F, G, H, I, K, L, M, N, P, Q, R, S, T, V, or W; V-243 may be replaced with A, C, D, E, F, G, H, I, K, L, M, N, P, Q, R, S, T, W or Y; E-244 may be replaced with A, C, D, F, G, H, I, K, L, M, N, P, Q, R, S, T, V, W or Y; T-245 may be replaced with A, C, D, E, F, G, H, I, K, L, M, N, P, Q, R, S, V, W or Y; K-449 may be replaced with A, C, D, E, F, G, H, I, L, M, N, P, Q, R, S, T, V, W or Y; P-450 may be replaced with A, C, D, E, F, G, H, I, K, L, M, N, Q, R, S, T, V, W or Y; Q-451 may be replaced with A, C, D, E, F, G, H, I, K, L, M, N, P, R, S, T, V, W or Y; N-452 may be replaced with A, C, D, E, F, G, H, I, K, L, M, P, Q, R, S, T, V, W or Y; P-453 may be replaced with A, C, D, E, F, G, H, I, K, L, M, N, Q, R, S, T, V, W or Y; I-454 may be replaced with A, C, D, E, F, G, H, K, L, M, N, P, Q, R, S, T, V, W or Y; Q-455 may be replaced with A, C, D, E, F, G, H, I, K, L, M, N, P, R, S, T, V, W or Y; L-456 may be replaced with A, C, D, E, F, G, H, I, K, M, N, P, Q, R, S, T, V, W or Y; P-457 may be replaced with A, C, D, E, F, G, H, I, K, L, M, N, Q, R, S, T, V, W or Y; G-458 may be replaced with A, C, D, E, F, H, I, K, L, M, N, P, Q, R, S, T, V, W or Y; D-459 may be replaced with A, C, E, F, G, H, I, K, L, M, N, P, Q, R, S, T, V, W or Y; L-460 may be replaced with A, C, D, E, F, G, H, I, K, M, N, P, Q, R, S, T, V, W or Y; P-461 may be replaced with A, C, D, E, F, G, H, I, K, L, M, N, Q, R, S, T, V, W or Y; G-462 may be replaced with A, C, D, E, F, H, I, K, L, M, N, P, Q, R, S, T, V, W or Y; T-463 may be replaced with A, C, D, E, F, G, H, I, K, L, M, N, P, Q, R, S, V, W or Y; S-464 may be replaced with A, C, D, E, F, G, H, I, K, L, M, N, P, Q, R, T, V, W or Y; Y-465 may be replaced with A, C, D, E, F, G, H, I, K, L, M, N, P, Q, R, S, T, V, or W; D-466 may be replaced with A, C, E, F, G, H, I, K, L, M, N, P, Q, R, S, T, V, W or Y; A-467 may be replaced with C, D, E, F, G, H, I, K, L, M, N, P, Q, R, S, T, V, W or Y; N-468 may be replaced with A, C, D, E, F, G, H, I, K, L, M, P, Q, R, S, T, V, W or Y; R-469 may be replaced with A, C, D, E, F, G, H, I, K, L, M, N, P, Q, S, T, V, W or Y; R-534 may be replaced with A, C, D, E, F, G, H, I, K, L, M, N, P, Q, S, T, V, W or Y; K-535 may be replaced with A, C, D, E, F, G, H, I, L, M, N, P, Q, R, S, T, V, W or Y; H-536 may be replaced with A, C, D, E, F, G, I, K, L, M, N, P, Q, R, S, T, V, W or Y; F-537 may be replaced with A, C, D, E, G, H, I, K, L, M, N, P, Q, R, S, T, V, W or Y; D-538 may be replaced with A, C, E, F, G, H, I, K, L, M, N, P, Q, R, S, T, V, W or Y; T-539 may be replaced with A, C, D, E, F, G, H, I, K, L, M, N, P, Q, R, S, V, W or Y; P-540 may be replaced with A, C, D, E, F, G, H, I, K, L, M, N, Q, R, S, T, V, W or Y; F-541 may be replaced with A, C, D, E, G, H, I, K, L, M, N, P, Q, R, S, T, V, W or Y; H-542 may be replaced with A, C, D, E, F, G, I, K, L, M, N, P, Q, R, S, T, V, W or Y; G-543 may be replaced with A, C, D, E, F, H, I, K, L, M, N, P, Q, R, S, T, V, W or Y; S-544 may be replaced with A, C, D, E, F, G, H, I, K, L, M, N, P, Q, R, T, V, W or Y; W-545 may be replaced with A, C, D, E, F, G, H, I, K, L, M, N, P, Q, R, S, T, V, or Y; G-546 may be replaced with A, C, D, E, F, H, I, K, L, M, N, P, Q, R, S, T, V, W or Y; M-547 may be replaced with A, C, D, E, F, G, H, I, K, L, N, P, Q, R, S, T, V, W or Y; W-548 may be replaced with A, C, D, E, F, G, H, I, K, L, M, N, P, Q, R, S, T, V, or Y; G-549 may be replaced with A, C, D, E, F, H, I, K, L, M, N, P, Q, R, S, T, V, W or Y; P-550 may be replaced with A, C, D, E, F, G, H, I, K, L, M, N, Q, R, S, T, V, W or Y; W-551 may be replaced with A, C, D, E, F, G, H, I, K, L, M, N, P, Q, R, S, T, V, or Y; G-552 may be replaced with A, C, D, E, F, H, I, K, L, M, N, P, Q, R, S, T, V, W or Y; D-553 may be replaced with A, C, E, F, G, H, I, K, L, M, N, P, Q, R, S, T, V, W or Y; G-554 may be replaced with A, C, D, E, F, H, I, K, L, M, N, P, Q, R, S, T, V, W or Y; S-831 may be replaced with A, C, D, E, F, G, H, I, K, L, M, N, P, Q, R, T, V, W or Y; F-832 may be replaced with A, C, D, E, G, H, I, K, L, M, N, P, Q, R, S, T, V, W or Y; N-833 may be replaced with A, C, D, E, F, G, H, I, K, L, M, P, Q, R, S, T, V, W or Y; A-834 may be replaced with C, D, E, F, G, H, I, K, L, M, N, P, Q, R, S, T, V, W or Y; I-835 may be replaced with A, C, D, E, F, G, H, K, L, M, N, P, Q, R, S, T, V, W or Y; P-836 may be replaced with A, C, D, E, F, G, H, I, K, L, M, N, Q, R, S, T, V, W or Y; T-837 may be replaced with A, C, D, E, F, G, H, I, K, L, M, N, P, Q, R, S, V, W or Y; F-838 may be replaced with A, C, D, E, G, H, I, K, L, M, N, P, Q, R, S, T, V, W or Y; S-839 may be replaced with A, C, D, E, F, G, H, I, K, L, M, N, P, Q, R, T, V, W or Y; A-840maybe replaced with C, D, E, F, G, H, I, K, L, M, N, P, Q, R, S, T, V, W or Y; W-841 may be replaced with A, C, D, E, F, G, H, I, K, L, M, N, P, Q, R, S, T, V or Y; V-842 may be replaced with A, C, D, E, F, G, H, I, K, L, M, N, P, Q, R, S, T, W or Y; I-843 may be replaced with A, C, D, E, F, G, H, K, L, M, N, P, Q, R, S, T, V, W or Y; E-844 may be replaced with A, C, D, F, G, H, I, K, L, M, N, P, Q, R, S, T, V, W or Y; E-845 may be replaced with A, C, D, F, G, H, I, K, L, M, N, P, Q, R, S, T, V, W or Y; W-846 may be replaced with A, C, D, E, F, G, H, I, K, L, M, N, P, Q, R, S, T, V, or Y; G-847 may be replaced with A, C, D, E, F, H, I, K, L, M, N, P, Q, R, S, T, V, W or Y; E-848 may be replaced with A, C, D, F, G, H, I, K, L, M, N, P, Q, R, S, T, V, W or Y; C-849 may be replaced with A, D, E, F, G, H, I, K, L, M, N, P, Q, R, S, T, V, W or Y; S-850 may be replaced with A, C, D, E, F, G, H, I, K, L, M, N, P, Q, R, T, V, W or Y; K-851 may be replaced with A, C, D, E, F, G, H, I, L, M, N, P, Q, R, S, T, V, W or Y; R-885 may be replaced with A, C, D, E, F, G, H, I, K, L, M, N, P, Q, S, T, V, W or Y; P-886 may be replaced with A, C, D, E, F, G, H, I, K, L, M, N, Q, R, S, T, V, W or Y; C-887 may be replaced with A, D, E, F, G, H, I, K, L, M, N, P, Q, R, S, T, V, W or Y; A-888 may be replaced with C, D, E, F, G, H, I, K, L, M, N, P, Q, R, S, T, V, W or Y; D-889 may be replaced with A, C, E, F, G, H, I, K, L, M, N, P, Q, R, S, T, V, W or Y; H-890 may be replaced with A, C, D, E, F, G, I, K, L, M, N, P, Q, R, S, T, V, W or Y; P-891 may be replaced with A, C, D, E, F, G, H, I, K, L, M, N, Q, R, S, T, V, W or Y; C-892 may be replaced with A, D, E, F, G, H, I, K, L, M, N, P, Q, R, S, T, V, W or Y; P-893 may be replaced with A, C, D, E, F, G, H, I, K, L, M, N, Q, R, S, T, V, W or Y; Q-894 may be replaced with A, C, D, E, F, G, H, I, K, L, M, N, P, R, S, T, V, W or Y; W-895 may be replaced with A, C, D, E, F, G, H, I, K, L, M, N, P, Q, R, S, T, V, or Y; Q-896 may be replaced with A, C, D, E, F, G, H, I, K, L, M, N, P, R, S, T, V, W or Y; L-897 may be replaced with A, C, D, E, F, G, H, I, K, M, N, P, Q, R, S, T, V, W or Y; G-898 may be replaced with A, C, D, E, F, H, I, K, L, M, N, P, Q, R, S, T, V, W or Y; E-899 may be replaced with A, C, D, F, G, H, I, K, L, M, N, P, Q, R, S, T, V, W or Y; W-900 may be replaced with A, C, D, E, F, G, H, I, K, L, M, N, P, Q, R, S, T, V, or Y; S-901 may be replaced with A, C, D, E, F, G, H, I, K, L, M, N, P, Q, R, T, V, W or Y; S-902 may be replaced with A, C, D, E, F, G, H, I, K, L, M, N, P, Q, R, T, V, W or Y; C-903 may be replaced with A, D, E, F, G, H, I, K, L, M, N, P, Q, R, S, T, V, W or Y; S-904 may be replaced with A, C, D, E, F, G, H, I, K, L, M, N, P, Q, R, T, V, W or Y; and/or K-905 may be replaced with A, C, D, E, F, G, H, I, L, M, N, P, Q, R, S, T, V, W or Y.

In addition, the following amino acids may be replaced in METH2 with the following amino acids: L-14 may be replaced with A, C, D, E, F, G, H, I, K, M, N, P, Q, R, S, T, V, W or Y; L-15 maybe replaced with A, C, D, E, F, G, H, I, K, M, N, P, Q, R, S, T, V, W or Y; L-16 may be replaced with A, C, D, E, F, G, H, I, K, M, N, P, Q, R, S, T, V, W or Y; L-17 may be replaced with A, C, D, E, F, G, H, I, K, M, N, P, Q, R, S, T, V, W or Y; L-18 may be replaced with A, C, D, E, F, G, H, I, K, M, N, P, Q, R, S, T, V, W or Y; L-19 may be replaced with A, C, D, E, F, G, H, I, K, M, N, P, Q, R, S, T, V, W or Y; L-20 may be replaced with A, C, D, E, F, G, H, I, K, M, N, P, Q, R, S, T, V, W or Y; L-21 may be replaced with A, C, D, E, F, G, H, I, K, M, N, P, Q, R, S, T, V, W or Y; L-22 may be replaced with A, C, D, E, F, G, H, I, K, M, N, P, Q, R, S, T, V, W or Y; P- 23 may be replaced with A, C, D, E, F, G, H, I, K, L, M, N, Q, R, S, T, V, W or Y; L-24 may be replaced with A, C, D, E, F, G, H, I, K, M, N, P, Q, R, S, T, V, W or Y; A-25 may be replaced with C, D, E, F, G, H, I, K, L, M, N, P, Q, R, S, T, V, W or Y; R-26 may be replaced with A, C, D, E, F, G, H, I, K, L, M, N, P, Q, S, T, V, W or Y; G-27 may be replaced with A, C, D, E, F, H, I, K, L, M, N, P, Q, R, S, T, V, W or Y; A-28 may be replaced with C, D, E, F, G, H, I, K, L, M, N, P, Q, R, S, T, V, W or Y; P-29 may be replaced with A, C, D, E, F, G, H, I, K, L, M, N, Q, R, S, T, V, W or Y; A-30 may be replaced with C, D, E, F, G, H, I, K, L, M, N, P, Q, R, S, T, V, W or Y; R-31 may be replaced with A, C, D, E, F, G, H, I, K, L, M, N, P, Q, S, T, V, W or Y; P-32 may be replaced with A, C, D, E, F, G, H, I, K, L, M, N, Q, R, S, T, V, W or Y; A-33 may be replaced with C, D, E, F, G, H, I, K, L, M, N, P, Q, R, S, T, V, W or Y; A-34 may be replaced with C, D, E, F, G, H, I, K, L, M, N, P, Q, R, S, T, V, W or Y; P-204 may be replaced with A, C, D, E, F, G, H, I, K, L, M, N, Q, R, S, T, V, W or Y; P-205 may be replaced with A, C, D, E, F, G, H, I, K, L, M, N, Q, R, S, T, V, W or Y; L-206 may be replaced with A, C, D, E, F, G, H, I, K, M, N, P, Q, R, S, T, V, W or Y; G-207 may be replaced with A, C, D, E, F, H, I, K, L, M, N, P, Q, R, S, T, V, W or Y; A-208 may be replaced with C, D, E, F, G, H, I, K, L, M, N, P, Q, R, S, T, V, W or Y; T-209 may be replaced with A, C, D, E, F, G, H, I, K, L, M, N, P, Q, R, S, V, W or Y; S-210 may be replaced with A, C, D, E, F, G, H, I, K, L, M, N, P, Q, R, T, V, W or Y; R-211 maybe replaced with A, C, D, E, F, G, H, I, K, L, M, N, P, Q, S, T, V, W or Y; T-212 may be replaced with A, C, D, E, F, G, H, I, K, L, M, N, P, Q, R, S, V, W or Y; K-213 may be replaced with A, C, D, E, F, G, H, I, L, M, N, P, Q, R, S, T, V, W or Y; R-214maybe replaced with A, C, D, E, F, G, H, I, K, L, M, N, P, Q, S, T, V, W or Y; F-215 may be replaced with A, C, D, E, G, H, I, K, L, M, N, P, Q, R, S, T, V, W or Y; V-216 may be replaced with A, C, D, E, F, G, H, I, K, L, M, N, P, Q, R, S, T, W or Y; S-217 may be replaced with A, C, D, E, F, G, H, I, K, L, M, N, P, Q, R, T, V, W or Y; E-218 may be replaced with A, C, D, F, G, H, I, K, L, M, N, P, Q, R, S, T, V, W or Y; A-219 may be replaced with C, D, E, F, G, H, I, K, L, M, N, P, Q, R, S, T, V, W or Y; R-220 may be replaced with A, C, D, E, F, G, H, I, K, L, M, N, P, Q, S, T, V, W or Y; F-221 may be replaced with A, C, D, E, G, H, I, K, L, M, N, P, Q, R, S, T, V, W or Y; V-222 may be replaced with A, C, D, E, F, G, H, I, K, L, M, N, P, Q, R, S, T, W or Y; E-223 may be replaced with A, C, D, F, G, H, I, K, L, M, N, P, Q, R, S, T, V, W or Y; T-224 may be replaced with A, C, D, E, F, G, H, I, K, L, M, N, P, Q, R, S, V, W or Y; P-430 may be replaced with A, C, D, E, F, G, H, I, K, L, M, N, Q, R, S, T, V, W or Y; G-431 may be replaced with A, C, D, E, F, H, I, K, L, M, N, P, Q, R, S, T, V, W or Y; A-432 may be replaced with C, D, E, F, G, H, I, K, L, M, N, P, Q, R, S, T, V, W or Y; A-433 may be replaced with C, D, E, F, G, H, I, K, L, M, N, P, Q, R, S, T, V, W or Y; L-434 may be replaced with A, C, D, E, F, G, H, I, K, M, N, P, Q, R, S, T, V, W or Y; P-435 may be replaced with A, C, D, E, F, G, H, I, K, L, M, N, Q, R, S, T, V, W or Y; L-436 may be replaced with A, C, D, E, F, G, H, I, K, M, N, P, Q, R, S, T, V, W or Y; P-437 may be replaced with A, C, D, E, F, G, H, I, K, L, M, N, Q, R, S, T, V, W or Y; T-438 may be replaced with A, C, D, E, F, G, H, I, K, L, M, N, P, Q, R, S, V, W or Y; G-439 may be replaced with A, C, D, E, F, H, I, K, L, M, N, P, Q, R, S, T, V, W or Y; L-440 may be replaced with A, C, D, E, F, G, H, I, K, M, N, P, Q, R, S, T, V, W or Y; P-441 may be replaced with A, C, D, E, F, G, H, I, K, L, M, N, Q, R, S, T, V, W or Y; G-442 may be replaced with A, C, D, E, F, H, I, K, L, M, N, P, Q, R, S, T, V, W or Y; R-443 may be replaced with A, C, D, E, F, G, H, I, K, L, M, N, P, Q, S, T, V, W or Y; M-444 may be replaced with A, C, D, E, F, G, H, I, K, L, N, P, Q, R, S, T, V, W or Y; A-445 may be replaced with C, D, E, F, G, H, I, K, L, M, N, P, Q, R, S, T, V, W or Y; L-446 may be replaced with A, C, D, E, F, G, H, I, K, M, N, P, Q, R, S, T, V, W or Y; Y-447 may be replaced with A, C, D, E, F, G, H, I, K, L, M, N, P, Q, R, S, T, V or W; Q-448 may be replaced with A, C, D, E, F, G, H, I, K, L, M, N, P, R, S, T, V, W or Y; L-449 may be replaced with A, C, D, E, F, G, H, I, K, M, N, P, Q, R, S, T, V, W or Y; D-450 may be replaced with A, C, E, F, G, H, I, K, L, M, N, P, Q, R, S, T, V, W or Y; E-520 may be replaced with A; C, D, F, G, H, I, K, L, M, N, P, Q, R, S, T, V, W or Y; R-521 may be replaced with A, C, D, E, F, G, H, I, K, L, M, N, P, Q, S, T, V, W or Y; P-522 may be replaced with A, C, D, E, F, G, H, I, K, L, M, N, Q, R, S, T, V, W or Y; K-523 may be replaced with A, C, D, E, F, G, H, I, L, M, N, P, Q, R, S, T, V, W or Y; P-524 may be replaced with A, C, D, E, F, G, H, I, K, L, M, N, Q, R, S, T, V, W or Y; V-525 may be replaced with A, C, D, E, F, G, H, I, K, L, M, N, P, Q, R, S, T, W or Y; V-526 may be replaced with A, C, D, E, F, G, H, I, K, L, M, N, P, Q, R, S, T, W or Y; D-527 may be replaced with A, C, E, F, G, H, I, K, L, M, N, P, Q, R, S, T, V, W or Y; G-528 may be replaced with A, C, D, E, F, H, I, K, L, M, N, P, Q, R, S, T, V, W or Y; G-529 may be replaced with A, C, D, E, F, H, I, K, L, M, N, P, Q, R, S, T, V, W or Y; W-530 may be replaced with A, C, D, E, F, G, H, I, K, L, M, N, P, Q, R, S, T, V, or Y; A-531 may be replaced with C, D, E, F, G, H, I, K, L, M, N, P, Q, R, S, T, V, W or Y; P-532 may be replaced with A, C, D, E, F, G, H, I, K, L, M, N, Q, R, S, T, V, W or Y; W-533 may be replaced with A, C, D, E, F, G, H, I, K, L, M, N, P, Q, R, S, T, V, or Y; G-534 may be replaced with A, C, D, E, F, H, I, K, L, M, N, P, Q, R, S, T, V, W or Y; P-535 may be replaced with A, C, D, E, F, G, H, I, K, L, M, N, Q, R, S, T, V, W or Y; W-536 may be replaced with A, C, D, E, F, G, H, I, K, L, M, N, P, Q, R, S, T, V or Y; G-537 may be replaced with A, C, D, E, F, H, I, K, L, M, N, P, Q, R, S, T, V, W or Y; E-538 may be replaced with A, C, D, F, G, H, I, K, L, M, N, P, Q, R, S, T, V, W or Y; C-539 may be replaced with A, D, E, F, G, H, I, K, L, M, N, P, Q, R, S, T, V, W or Y; S-540 may be replaced with A, C, D, E, F, G, H, I, K, L, M, N, P, Q, R, T, V, W or Y; N-827 may be replaced with A, C, D, E, F, G, H, I, K, L, M, P, Q, R, S, T, V, W or Y; I-828 may be replaced with A, C, D, E, F, G, H, K, L, M, N, P, Q, R, S, T, V, W or Y; I-829 may be replaced with A, C, D, E, F, G, H, K, L, M, N, P, Q, R, S, T, V, W or Y; Q-830 may be replaced with A, C, D, E, F, G, H, I, K, L, M, N, P, R, S, T, V, W or Y; P-831 may be replaced with A, C, D, E, F, G, H, I, K, L, M, N, Q, R, S, T, V, W or Y; L-832 may be replaced with A, C, D, E, F, G, H, I, K, M, N, P, Q, R, S, T, V, W or Y; L-833 may be replaced with A, C, D, E, F, G, H, I, K, M, N, P, Q, R, S, T, V, W or Y; H-834 may be replaced with A, C, D, E, F, G, I, K, L, M, N, P, Q, R, S, T, V, W or Y; A-835 may be replaced with C, D, E, F, G, H, I, K, L, M, N, P, Q, R, S, T, V, W or Y; Q-836 may be replaced with A, C, D, E, F, G, H, I, K, L, M, N, P, R, S, T, V, W or Y; W-837 may be replaced with A, C, D, E, F, G, H, I, K, L, M, N, P, Q, R, S, T, V, or Y; V-838 may be replaced with A, C, D, E, F, G, H, I, K, L, M, N, P, Q, R, S, T, W or Y; L-839 may be replaced with A, C, D, E, F, G, H, I, K, M, N, P, Q, R, S, T, V, W or Y; G-840 may be replaced with A, C, D, E, F, H, I, K, L, M, N, P, Q, R, S, T, V, W or Y; D-841 may be replaced with A, C, E, F, G, H, I, K, L, M, N, P, Q, R, S, T, V, W or Y; W-842 may be replaced with A, C, D, E, F, G, H, I, K, L, M, N, P, Q, R, S, T, V or Y; S-843 may be replaced with A, C, D, E, F, G, H, I, K, L, M, N, P, Q, R, T, V, W or Y; E-844 may be replaced with A, C, D, F, G, H, I, K, L, M, N, P, Q, R, S, T, V, W or Y; C-845 may be replaced with A, D, E, F, G, H, I, K, L, M, N, P, Q, R, S, T, V, W or Y; S-846 may be replaced with A, C, D, E, F, G, H, I, K, L, M, N, P, Q, R, T, V, W or Y; and/or S-847 may be replaced with A, C, D, E, F, G, H, I, K, L, M, N, P, Q, R, T, V, W or Y.

METH1 or METH2 polypeptide variants, including substitution, deletion and/or addition variants, which contain amino acid substitutions can be tested for activity in any of the assays described herein, for example, the chorioallantoic assay or the cornea pocket assay. Preferred are METH1 or METH2 polypeptides with conservative substitutions that: maintain all the activities and/or properties of the wild type protein; or have one or more enhanced activities and/or properties compared to the wild type protein. Also preferred are METH1 or METH2 polypeptides with nonconservative substitutions which: lack an activity and/or property of the wild type protein, while maintaining all other activities and/or properties; or lack more than one activity and/or property of the wild type protein.

For example, activities or properties of METH1 or METH2 that may be altered in METH1 or METH2 polypeptides with conservative or nonconservative substitutions include, but are not limited to: stimulation of angiogenesis; stimulation of epithelial cell proliferation; antibody binding; ligand binding; stability; solubility; and/or properties which affect purification.

The polypeptides of the present invention are preferably provided in an isolated form. By “isolated polypeptide” is intended a polypeptide removed from its native environment. Thus, a polypeptide produced and/or contained within a recombinant host cell is considered isolated for purposes of the present invention. Also intended as an “isolated polypeptide” are polypeptides that have been purified, partially or substantially, from a recombinant host cell or from a native source. For example, a recombinantly produced version of the METH1 or METH2 polypeptide can be substantially purified by the one-step method described in Smith and Johnson, Gene 67:31-40 (1988).

The polypeptides of the present invention include the METH1 polypeptide encoded by the deposited cDNA including the leader; the mature METH1 polypeptide encoded by the deposited the cDNA minus the leader (i.e., the mature protein); a polypeptide comprising amino acids about 1 to about 950 in SEQ ID NO:2; a polypeptide comprising amino acids about 2 to about 950 in SEQ ID NO:2; a polypeptide comprising amino acids about 29 to about 950 in SEQ ID NO:2; a polypeptide comprising amino acids about 30 to about 950 in SEQ ID NO:2; a polypeptide comprising the metalloprotease domain of METH1, amino acids 235 to 459 in SEQ ID NO:2; a polypeptide comprising the disintegrin domain of METH1, amino acids 460 to 544 in SEQ ID NO:2; a polypeptide comprising the first TSP-like domain of METH1, amino acids 545 to 598 in SEQ ID NO:2; a polypeptide comprising the second TSP-like domain of METH1, amino acids 841 to 894 in SEQ ID NO:2; a polypeptide comprising the third TSP-like domain of METH1, amino acids 895 to 934 in SEQ ID NO:2; a polypeptide comprising amino acids 536 to 613 in SEQ ID NO:2; a polypeptide comprising amino acids 549 to 563 in SEQ ID NO:2; the METH2 polypeptide encoded by the deposited cDNA including the leader; the mature METH2 polypeptide encoded by the deposited the cDNA minus the leader (i.e., the mature protein); a polypeptide comprising amino acids about 1 to about 890 in SEQ ID NO:4; a polypeptide comprising amino acids about 2 to about 890 in SEQ ID NO:4; a polypeptide comprising amino acids about 24 to about 890 in SEQ ID NO:4; a polypeptide comprising amino acids about 112 to about 890 in SEQ ID NO:4; a polypeptide comprising the metalloprotease domain of METH2, amino acids 214 to 439 in SEQ ID NO:4; a polypeptide comprising the disintegrin domain of METH2, amino acids 440 to 529 in SEQ ID NO:4; a polypeptide comprising the first TSP-like domain of METH2, amino acids 530 to 583 in SEQ ID NO:4; a polypeptide comprising the second TSP-like domain of METH2, amino acids 837 to 890 in SEQ ID NO:4; a polypeptide comprising amino acids 280 to 606 in SEQ ID NO:4; a polypeptide comprising amino acids 529 to 548 in SEQ ID NO:4; as well as polypeptides which are at least 80% identical, and more preferably at least 85%, 90%, 91%, 92%, 93%, 94%, 95%, 96%, 97%, 98% or 99% identical to the polypeptides described above and also include portions of such polypeptides with at least 30 amino acids and more preferably at least 50 amino acids.

By a polypeptide having an amino acid sequence at least, for example, 95% “identical” to a reference amino acid sequence of a METH1 or METH2 polypeptide is intended that the amino acid sequence of the polypeptide is identical to the reference sequence except that the polypeptide sequence may include up to five amino acid alterations per each 100 amino acids of the reference amino acid of the METH1 or METH2 polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 91%, 92%, 93%, 94%, 95%, 96%, 97%, 98% or 99% identical to, for instance, the amino acid sequence shown in SEQ ID NO:2 or SEQ ID NO:4 or to the amino acid sequence encoded by deposited cDNA clones can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

A preferred method for determining the best overall match between a query sequence (a sequence of the present invention) and a subject sequence, also referred to as a global sequence alignment, can be determined using the FASTDB computer program based on the algorithm of Brutlag et al., Comp. App. Biosci. 6:237-245 (1990). In a sequence alignment, the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group Length=O, Cutoff Score=1, Gap Penalty=5, Gap Size Penalty=0.05, Window Size=500 or the length of the subject amino acid sequence, whichever is shorter.

If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total residues of the query sequence. Whether a residue is matched/aligned is determined by the results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the FASTDB alignment does not show a match/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched, the final percent identity would be 90%. In another example, a 90 residue subject sequence is compared with a 100 residue query sequence. This time, the deletions are internal, so there are no residues at the - or C-termini of the subject sequence which are not matched/aligned with the query. In this case, the percent identity calculated by FASTDB is not manually corrected. Once again, only residue positions outside the - and C-terminal ends of the subject sequence, as displayed in the FASTDB alignment, which are not matched/aligned with the query sequence are manually corrected for. No other manual corrections are made for the purposes of the present invention.

The polypeptides of the present invention are useful as a molecular weight marker on SDS-PAGE gels or on molecular sieve gel filtration columns using methods well known to those of skill in the art.

In another aspect, the invention provides a peptide or polypeptide comprising an epitope-bearing portion of a polypeptide of the invention. The epitope of this polypeptide portion is an immunogenic or antigenic epitope of a polypeptide described herein. An “immunogenic epitope” is defined as a part of a protein that elicits an antibody response when the whole protein is the immunogen. On the other hand, a region of a protein molecule to which an antibody can bind is defined as an “antigenic epitope.” The number of immunogenic epitopes of a protein generally is less than the number of antigenic epitopes. See, for instance, Geysen et al., Proc. Natl. Acad. Sci. USA 81:3998-4002 (1983).

As to the selection of peptides or polypeptides bearing an antigenic epitope (i.e., that contain a region of a protein molecule to which an antibody can bind), it is well known in the art that relatively short synthetic peptides that mimic part of a protein sequence are routinely capable of eliciting an antiserum that reacts with the partially mimicked protein. See, for instance, Sutcliffe, J. G. et al., “Antibodies that react with predetermined sites on proteins”, Science 219:660-666 (1983). Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals.

Antigenic epitope-bearing peptides and polypeptides of the invention are therefore useful to raise antibodies, including monoclonal antibodies, that bind specifically to a polypeptide of the invention. See, for instance, Wilson et al., Cell 37:767-778 (1984) at 777.

Antigenic epitope-bearing peptides and polypeptides of the invention preferably contain a sequence of at least seven, more preferably at least nine and most preferably between about at least about 15 to about 30 amino acids contained within the amino acid sequence of a polypeptide of the invention.

The epitope-bearing peptides and polypeptides of the invention may be produced by any conventional means. Houghten, R. A., “General method for the rapid solid-phase synthesis of large numbers of peptides: specificity of antigen-antibody interaction at the level of individual amino acids”, Proc. Natl. Acad. Sci. USA 82:5131-5135 (1985). This “Simultaneous Multiple Peptide Synthesis (SMPS)” process is further described in U.S. Pat. No. 4,631,211 to Houghten et al. (1986).

As one of skill in the art will appreciate, METH1 or METH2 polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EPA 394,827; Traunecker et al., Nature 331:84-86 (1988)). Fusion proteins that have a disulfide-linked dimeric structure due to the IgG part can also be more efficient in binding and neutralizing other molecules than the monomeric METH1 or METH2 protein or protein fragment alone (Fountoulakis et al., J. Biochem. 270:3958-3964 (1995)).

METH1 and METH2 Polynucleotide and Polypeptide Fragments

In the present invention, a “polynucleotide fragment” refers to a short polynucleotide having a nucleic acid sequence contained in the deposited clones or shown in SEQ ID NO: 1 or SEQ ID NO:3. The short nucleotide fragments are preferably at least about 15 nt, and more preferably at least about 20 nt, still more preferably at least about 30 nt, and even more preferably, at least about 40 nt in length. A fragment “at least 20 nt in length,” for example, is intended to include 20 or more contiguous bases from the cDNA sequence contained in the deposited clones or the nucleotide sequence shown in SEQ ID NO:1 or SEQ ID NO:3. These nucleotide fragments are useful as diagnostic probes and primers as discussed herein. Of course, larger fragments (e.g., 50, 150, 500, 600, 2000 nucleotides) are preferred.

Moreover, representative examples of METH1 or METH2 polynucleotide fragments include, for example, fragments having a sequence from about nucleotide number 1-50, 51-100, 101-150, 151-200, 201-250, 251-300, 301-350, 351-400, 401-450, 451-500, 501-550, 551-600, 651-700, 701-750, 751-800, 800-850, 851-900, 901-950, 951-1000, 1001-1050, 1051-1100, 1101-1150, 1151-1200, 1201-1250, 1251-1300, 1301-1350, 1351-1400, 1401-1450, 1451-1500, 1501-1550, 1551-1600, 1601-1650, 1651-1700, 1701-1750, 1751-1800, 1801-1850, 1851-1900, 1901-1950, 1951-2000, or 2001 to the end of SEQ ID NO: 1 or SEQ ID NO:3 or the cDNA contained in the deposited clones. In this context “about” includes the particularly recited ranges, larger or smaller by several (5,4, 3, 2, or 1) nucleotides, at either terminus or at both termini. Preferably, these fragments encode a polypeptide which has biological activity. More preferably, these polynucleotides can be used as probes or primers as discussed herein.

In the present invention, a “polypeptide fragment” refers to a short amino acid sequence contained in SEQ ID NO:2 or SEQ ID NO:4 or encoded by the cDNA contained in the deposited clones. Protein fragments may be “free-standing,” or comprised within a larger polypeptide of which the fragment forms a part or region, most preferably as a single continuous region. Representative examples of polypeptide fragments of the invention, include, for example, fragments from about amino acid number 1-20, 21-40, 41-60, 61-80, 81-100, 102-120, 121-140, 141-160, 161-180, 181-200,201-220, 221-240, 241-260,261-280, or 281 to the end of the coding region of SEQ ID NO:2 or SEQ ID NO:4. Moreover, polypeptide fragments can be about 20, 30, 40, 50, 60, 70, 80, 90, 100, 110, 120, 130, 140, or 150 amino acids in length. In this context “about” includes the particularly recited ranges, larger or smaller by several (5, 4, 3, 2, or 1) amino acids, at either extreme or at both extremes.

Preferred polypeptide fragments include the secreted METH1 or METH2 protein as well as the mature form. Further preferred polypeptide fragments include the secreted METH1 or METH2 protein or the mature form having a continuous series of deleted residues from the amino or the carboxy terminus, or both. For example, any number of amino acids, ranging from 1-60, can be deleted from the amino terminus of either the secreted METH1 or METH2 polypeptide or the mature form. Similarly, any number of amino acids, ranging from 1-30, can be deleted from the carboxy terminus of the secreted METH1 or METH2 protein or mature form. Furthermore, any combination of the above amino and carboxy terminus deletions are preferred. Similarly, polynucleotide fragments encoding these METH1 or METH2 polypeptide fragments are also preferred.

Particularly, N-terminal deletions of the METH1 polypeptide can be described by the general formula m⁻⁹⁵⁰, where m is an integer from 2 to 949, where m corresponds to the position of the amino acid residue identified in SEQ ID NO:2. Preferably, N-terminal deletions of the METH1 polypeptide of the invention shown as SEQ ID NO:2 include polypeptides comprising the amino acid sequence of residues: G-2 to S-950; N-3 to S-950; A-4 to S-950; E-5 to S-950; R-6 to S-950; A-7 to S-950; P-8 to S-950; G-9 to S-950; S-10 to S-950; R-11 to S-950; S-12 to S-950; F-13 to S-950; G-14 to S-950; P-15 to S-950; V-16 to S-950; P-17 to S-950; T-18 to S-950; L-19 to S-950; L-20 to S-950; L-21 to S-950; L-22 to S-950; A-23 to S-950; A-24 to S-950; A-25 to S-950; L-26 to S-950; L-27 to S-950; A-28 to S-950; V-29 to S-950; S-30 to S-950; D-31 to S-950; A-32 to S-950; L-33 to S-950; G-34 to S-950; R-35 to S-950; P-36 to S-950; S-37 to S-950; E-38 to S-950; E-39 to S-950; D-40 to S-950; E-41 to S-950; E-42 to S-950; L-43 to S-950; V-44 to S-950; V-45 to S-950; P-46 to S-950; E-47 to S-950; L-48 to S-950; E-49 to S-950; R-50 to S-950; A-51 to S-950; P-52 to S-950; G-53 to S-950; H-54 to S-950; G-55 to S-950; T-56 to S-950; T-57 to S-950; R-58 to S-950; L-59 to S-950; R-60 to S-950; L-61 to S-950; H-62 to S-950; A-63 to S-950; F-64 to S-950; D-65 to S-950; Q-66 to S-950; Q-67 to S-950; L-68 to S-950; D-69 to S-950; L-70 to S-950; E-71 to S-950; L-72 to S-950; R-73 to S-950; P-74 to S-950; D-75 to S-950; S-76 to S-950; S-77 to S-950; F-78 to S-950; L-79 to S-950; A-80 to S-950; P-81 to S-950; G-82 to S-950; F-83 to S-950; T-84 to S-950; L-85 to S-950; Q-86 to S-950; N-87 to S-950; V-88 to S-950; G-89 to S-950; R-90 to S-950; K-91 to S-950; S-92 to S-950; G-93 to S-950; S-94 to S-950; E-95 to S-950; T-96 to S-950; P-97 to S-950; L-98 to S-950; P-99 to S-950; E-100 to S-950; T-101 to S-950; D-102 to S-950; L-103 to S-950; A-104 to S-950; H-105 to S-950; C-106 to S-950; F-107 to S-950; Y-108 to S-950; S-109 to S-950; G-110 to S-950; T-111 to S-950; V-112 to S-950; N-113 to S-950; G-114 to S-950; D-115 to S-950; P-116 to S-950; S-117 to S-950; S-118 to S-950; A-19 to S-950; A-120 to S-950; A-121 to S-950; L-122 to S-950; S-123 to S-950; L-124 to S-950; C-125 to S-950; E-126 to S-950; G-127 to S-950; V-128 to S-950; R-129 to S-950; G-130 to S-950; A-131 to S-950; F-132 to S-950; Y-133 to S-950; L-134 to S-950; L-135 to S-950; G-136 to S-950; E-137 to S-950; A-138 to S-950; Y-139 to S-950; F-140 to S-950; I-141 to S-950; Q-142 to S-950; P-143 to S-950; L-144 to S-950; P-145 to S-950; A-146 to S-950; A-147 to S-950; S-148 to S-950; E-149 to S-950; R-150 to S-950; L-151 to S-950; A-152 to S-950; T-153 to S-950; A-154 to S-950; A-155 to S-950; P-156 to S-950; G-157 to S-950; E-158 to S-950; K-159 to S-950; P-160 to S-950; P-161 to S-950; A-162 to S-950; P-163 to S-950; L-164 to S-950; Q-165 to S-950; F-166 to S-950; H-167 to S-950; L-168 to S-950; L-169 to S-950; R-170 to S-950; R-171 to S-950; N-172 to S-950; R-173 to S-950; Q-174 to S-950; G-175 to S-950; D-176 to S-950; V-177 to S-950; G-178 to S-950; G-179 to S-950; T-180 to S-950; C-181 to S-950; G-182 to S-950; V-183 to S-950; V-184 to S-950; D-185 to S-950; D-186 to S-950; E-187 to S-950; P-188 to S-950; R-189 to S-950; P-190 to S-950; T-191 to S-950; G-192 to S-950; K-193 to S-950; A-194 to S-950; E-195 to S-950; T-196 to S-950; E-197 to S-950; D-198 to S-950; E-199 to S-950; D-200 to S-950; E-201 to S-950; G-202 to S-950; T-203 to S-950; E-204 to S-950; G-205 to S-950; E-206 to S-950; D-207 to S-950; E-208 to S-950; G-209 to S-950; P-210 to S-950; Q-211 to S-950; W-212 to S-950; S-213 to S-950; P-214 to S-950; Q-215 to S-950; D-216 to S-950; P-217 to S-950; A-218 to S-950; L-219 to S-950; Q-220 to S-950; G-221 to S-950; V-222 to S-950; G-223 to S-950; Q-224 to S-950; P-225 to S-950; T-226 to S-950; G-227 to S-950; T-228 to S-950; G-229 to S-950; S-230 to S-950; I-231 to S-950; R-232 to S-950; K-233 to S-950; K-234 to S-950; R-235 to S-950; F-236 to S-950; V-237 to S-950; S-238 to S-950; S-239 to S-950; H-240 to S-950; R-241 to S-950; Y-242 to S-950; V-243 to S-950; E-244 to S-950; T-245 to S-950; M-246 to S-950; L-247 to S-950; V-248 to S-950; A-249 to S-950; D-250 to S-950; Q-251 to S-950; S-252 to S-950; M-253 to S-950; A-254 to S-950; E-255 to S-950; F-256 to S-950; H-257 to S-950; G-258 to S-950; S-259 to S-950; G-260 to S-950; L-261 to S-950; K-262 to S-950; H-263 to S-950; Y-264 to S-950; L-265 to S-950; L-266 to S-950; T-267 to S-950; L-268 to S-950; F-269 to S-950; S-270 to S-950; V-271 to S-950; A-272 to S-950; A-273 to S-950; R-274 to S-950; L-275 to S-950; Y-276 to S-950; K-277 to S-950; H-278 to S-950; P-279 to S-950; S-280 to S-950; I-281 to S-950; R-282 to S-950; N-283 to S-950; S-284 to S-950; V-285 to S-950; S-286 to S-950; L-287 to S-950; V-288 to S-950; V-289 to S-950; V-290 to S-950; K-291 to S-950; I-292 to S-950; L-293 to S-950; V-294 to S-950; I-295 to S-950; H-296 to S-950; D-297 to S-950; E-298 to S-950; Q-299 to S-950; K-300 to S-950; G-301 to S-950; P-302 to S-950; E-303 to S-950; V-304 to S-950; T-305 to S-950; S-306 to S-950; N-307 to S-950; A-308 to S-950; A-309 to S-950; L-310 to S-950; T-311 to S-950; L-312 to S-950; R-313 to S-950; N-314 to S-950; F-315 to S-950; C-316 to S-950; N-317 to S-950; W-318 to S-950; Q-319 to S-950; K-320 to S-950; Q-321 to S-950; H-322 to S-950; N-323 to S-950; P-324 to S-950; P-325 to S-950; S-326 to S-950; D-327 to S-950; R-328 to S-950; D-329 to S-950; A-330 to S-950; E-331 to S-950; H-332 to S-950; Y-333 to S-950; D-334 to S-950; T-335 to S-950; A-336 to S-950; I-337 to S-950; L-338 to S-950; F-339 to S-950; T-340 to S-950; R-341 to S-950; Q-342 to S-950; D-343 to S-950; L-344 to S-950; C-345 to S-950; G-346 to S-950; S-347 to S-950; Q-348 to S-950; T-349 to S-950; C-350 to S-950; D-351 to S-950; T-352 to S-950; L-353 to S-950; G-354 to S-950; M-355 to S-950; A-356 to S-950; D-357 to S-950; V-358 to S-950; G-359 to S-950; T-360 to S-950; V-361 to S-950; C-362 to S-950; D-363 to S-950; P-364 to S-950; S-365 to S-950; R-366 to S-950; S-367 to S-950; C-368 to S-950; S-369 to S-950; V-370 to S-950; I-371 to S-950; E-372 to S-950; D-373 to S-950; D-374 to S-950; G-375 to S-950; L-376 to S-950; Q-377 to S-950; A-378 to S-950; A-379 to S-950; F-380 to S-950; T-381 to S-950; T-382 to S-950; A-383 to S-950; H-384 to S-950; E-385 to S-950; L-386 to S-950; G-387 to S-950; H-388 to S-950; V-389 to S-950; F-390 to S-950; N-391 to S-950; M-392 to S-950; P-393 to S-950; H-394 to S-950; D-395 to S-950; D-396 to S-950; A-397 to S-950; K-398 to S-950; Q-399 to S-950; C-400 to S-950; A-401 to S-950; S-402 to S-950; L-403 to S-950; N-404 to S-950; G-405 to S-950; V-406 to S-950; N-407 to S-950; Q-408 to S-950; D-409 to S-950; S-410 to S-950; H-411 to S-950; M-412 to S-950; M-413 to S-950; A-414 to S-950; S-415 to S-950; M-416 to S-950; L-417 to S-950; S-418 to S-950; N-419 to S-950; L-420 to S-950; D-421 to S-950; H-1422 to S-950; S-423 to S-950; Q-424 to S-950; P-425 to S-950; W-426 to S-950; S-427 to S-950; P-428 to S-950; C-429 to S-950; S-430 to S-950; A-431 to S-950; Y-432 to S-950; M-433 to S-950; I-434 to S-950; T-435 to S-950; S-436 to S-950; F-437 to S-950; L-438 to S-950; D-439 to S-950; N-440 to S-950; G-441 to S-950; H-442 to S-950; G-443 to S-950; E-444 to S-950; C-445 to S-950; L-446 to S-950; M-447 to S-950; D-448 to S-950; K-449 to S-950; P-450 to S-950; Q-451 to S-950; N-452 to S-950; P-453 to S-950; I-454 to S-950; Q-455 to S-950; L-456 to S-950; P-457 to S-950; G-458 to S-950; D-459 to S-950; L-460 to S-950; P-461 to S-950; G-462 to S-950; T-463 to S-950; S-464 to S-950; Y-465 to S-950; D-466 to S-950; A-467 to S-950; N-468 to S-950; R-469 to S-950; Q-470 to S-950; C-471 to S-950; Q-472 to S-950; F-473 to S-950; T-474 to S-950; F-475 to S-950; G-476 to S-950; E-477 to S-950; D-478 to S-950; S-479 to S-950; K-480 to S-950; H-481 to S-950; C-482 to S-950; P-483 to S-950; D-484 to S-950; A-485 to S-950; A-486 to S-950; S-487 to S-950; T-488 to S-950; C-489 to S-950; S-490 to S-950; T-491 to S-950; L-492 to S-950; W-493 to S-950; C-494 to S-950; T-495 to S-950; G-496 to S-950; T-497 to S-950; S-498 to S-950; G-499 to S-950; G-500 to S-950; V-501 to S-950; L-502 to S-950; V-503 to S-950; C-504 to S-950; Q-505 to S-950; T-506 to S-950; K-507 to S-950; H-508 to S-950; F-509 to S-950; P-510 to S-950; W-511 to S-950; A-512 to S-950; D-513 to S-950; G-514 to S-950; T-515 to S-950; S-516 to S-950; C-517 to S-950; G-518 to S-950; E-519 to S-950; G-520 to S-950; K-521 to S-950; W-522 to S-950; C-523 to S-950; I-524 to S-950; N-525 to S-950; G-526 to S-950; K-527 to S-950; C-528 to S-950; V-529 to S-950; N-530 to S-950; K-531 to S-950; T-532 to S-950; D-533 to S-950; R-534 to S-950; K-535 to S-950; H-536 to S-950; F-537 to S-950; D-538 to S-950; T-539 to S-950; P-540 to S-950; F-541 to S-950; H-542 to S-950; G-543 to S-950; S-544 to S-950; W-545 to S-950; G-546 to S-950; M-547 to S-950; W-548 to S-950; G-549 to S-950; P-550 to S-950; W-551 to S-950; G-552 to S-950; D-553 to S-950; C-554 to S-950; S-555 to S-950; R-556 to S-950; T-557 to S-950; C-558 to S-950; G-559 to S-950; G-560 to S-950; G-561 to S-950; V-562 to S-950; Q-563 to S-950; Y-564 to S-950; T-565 to S-950; M-566 to S-950; R-567 to S-950; E-568 to S-950; C-569 to S-950; D-570 to S-950; N-571 to S-950; P-572 to S-950; V-573 to S-950; P-574 to S-950; K-575 to S-950; N-576 to S-950; G-577 to S-950; G-578 to S-950; K-579 to S-950; Y-580 to S-950; C-581 to S-950; E-582 to S-950; G-583 to S-950; K-584 to S-950; R-585 to S-950; V-586 to S-950; R-587 to S-950; Y-588 to S-950; R-589 to S-950; S-590 to S-950; C-591 to S-950; N-592 to S-950; L-593 to S-950; E-594 to S-950; D-595 to S-950; C-596 to S-950; P-597 to S-950; D-598 to S-950; N-599 to S-950; N-600 to S-950; G-601 to S-950; K-602 to S-950; T-603 to S-950; F-604 to S-950; R-605 to S-950; E-606 to S-950; E-607 to S-950; Q-608 to S-950; C-609 to S-950; E-610 to S-950; A-611 to S-950; H-612 to S-950; N-613 to S-950; E-614 to S-950; F-615 to S-950; S-616 to S-950; K-617 to S-950; A-618 to S-950; S-619 to S-950; F-620 to S-950; G-621 to S-950; S-622 to S-950; G-623 to S-950; P-624 to S-950; A-625 to S-950; V-626 to S-950; E-627 to S-950; W-628 to S-950; I-629 to S-950; P-630 to S-950; K-631 to S-950; Y-632 to S-950; A-633 to S-950; G-634 to S-950; V-635 to S-950; S-636 to S-950; P-637 to S-950; K-638 to S-950; D-639 to S-950; R-640 to S-950; C-641 to S-950; K-642 to S-950; L-643 to S-950; I-644 to S-950; C-645 to S-950; Q-646 to S-950; A-647 to S-950; K-648 to S-950; G-649 to S-950; I-650 to S-950; G-651 to S-950; Y-652 to S-950; F-653 to S-950; F-654 to S-950; V-655 to S-950; L-656 to S-950; Q-657 to S-950; P-658 to S-950; K-659 to S-950; V-660 to S-950; V-661 to S-950; D-662 to S-950; G-663 to S-950; T-664 to S-950; P-665 to S-950; C-666 to S-950; S-667 to S-950; P-668 to S-950; D-669 to S-950; S-670 to S-950; T-671 to S-950; S-672 to S-950; V-673 to S-950; C-674 to S-950; V-675 to S-950; Q-676 to S-950; G-677 to S-950; Q-678 to S-950; C-679 to S-950; V-680 to S-950; K-681 to S-950; A-682 to S-950; G-683 to S-950; C-684 to S-950; D-685 to S-950; R-686 to S-950; I-687 to S-950; I-688 to S-950; D-689 to S-950; S-690 to S-950; K-691 to S-950; K-692 to S-950; K-693 to S-950; F-694 to S-950; D-695 to S-950; K-696 to S-950; C-697 to S-950; G-698 to S-950; V-699 to S-950; C-700 to S-950; G-701 to S-950; G-702 to S-950; N-703 to S-950; G-704 to S-950; S-705 to S-950; T-706 to S-950; C-707 to S-950; K-708 to S-950; K-709 to S-950; I-710 to S-950; S-711 to S-950; G-712 to S-950; S-713 to S-950; V-714 to S-950; T-715 to S-950; S-716 to S-950; A-717 to S-950; K-718 to S-950; P-719 to S-950; G-720 to S-950; Y-721 to S-950; H-722 to S-950; D-723 to S-950; I-724 to S-950; I-725 to S-950; T-726 to S-950; I-727 to S-950; P-728 to S-950; T-729 to S-950; G-730 to S-950; A-731 to S-950; T-732 to S-950; N-733 to S-950; I-734 to S-950; E-735 to S-950; V-736 to S-950; K-737 to S-950; Q-738 to S-950; R-739 to S-950; N-740 to S-950; Q-741 to S-950; R-742 to S-950; G-743 to S-950; S-744 to S-950; R-745 to S-950; N-746 to S-950; N-747 to S-950; G-748 to S-950; S-749 to S-950; F-750 to S-950; L-751 to S-950; A-752 to S-950; I-753 to S-950; K-754 to S-950; A-755 to S-950; A-756 to S-950; D-757 to S-950; G-758 to S-950; T-759 to S-950; Y-760 to S-950; I-761 to S-950; L-762 to S-950; N-763 to S-950; G-764 to S-950; D-765 to S-950; Y-766 to S-950; T-767 to S-950; L-768 to S-950; S-769 to S-950; T-770 to S-950; L-771 to S-950; E-772 to S-950; Q-773 to S-950; D-774 to S-950; I-775 to S-950; M-776 to S-950; Y-777 to S-950; K-778 to S-950; G-779 to S-950; V-780 to S-950; V-781 to S-950; L-782 to S-950; R-783 to S-950; Y-784 to S-950; S-785 to S-950; G-786 to S-950; S-787 to S-950; S-788 to S-950; A-789 to S-950; A-790 to S-950; L-791 to S-950; E-792 to S-950; R-793 to S-950; I-794 to S-950; R-795 to S-950; S-796 to S-950; F-797 to S-950; S-798 to S-950; P-799 to S-950; L-800 to S-950; K-801 to S-950; E-802 to S-950; P-803 to S-950; L-804 to S-950; T-805 to S-950; I-806 to S-950; Q-807 to S-950; V-808 to S-950; L-809 to S-950; T-810 to S-950; V-811 to S-950; G-812 to S-950; N-813 to S-950; A-814 to S-950; L-815 to S-950; R-816 to S-950; P-817 to S-950; K-818 to S-950; I-819 to S-950; K-820 to S-950; Y-821 to S-950; T-822 to S-950; Y-823 to S-950; F-824 to S-950; V-825 to S-950; K-826 to S-950; K-827 to S-950; K-828 to S-950; K-829 to S-950; E-830 to S-950; S-831 to S-950; F-832 to S-950; N-833 to S-950; A-834 to S-950; I-835 to S-950; P-836 to S-950; T-837 to S-950; F-838 to S-950; S-839 to S-950; A-840 to S-950; W-841 to S-950; V-842 to S-950; I-843 to S-950; E-844 to S-950; E-845 to S-950; W-846 to S-950; G-847 to S-950; E-848 to S-950; C-849 to S-950; S-850 to S-950; K-851 to S-950; S-852 to S-950; : C-853 to S-950; E-854 to S-950; L-855 to S-950; G-856 to S-950; W-857 to S-950; Q-858 to S-950; R-859 to S-950; R-860 to S-950; L-861 to S-950; V-862 to S-950; E-863 to S-950; C-864 to S-950; R-865 to S-950; D-866 to S-950; I-867 to S-950; N-868 to S-950; G-869 to S-950; Q-870 to S-950; P-871 to S-950; A-872 to S-950; S-873 to S-950; E-874 to S-950; C-875 to S-950; A-876 to S-950; K-877 to S-950; E-878 to S-950; V-879 to S-950; K-880 to S-950; P-881 to S-950; A-882 to S-950; S-883 to S-950; T-884 to S-950; R-885 to S-950; P-886 to S-950; C-887 to S-950; A-888 to S-950; D-889 to S-950; H-890 to S-950; P-891 to S-950; C-892 to S-950; P-893 to S-950; Q-894 to S-950; W-895 to S-950; Q-896 to S-950; L-897 to S-950; G-898 to S-950; E-899 to S-950; W-900 to S-950; S-901 to S-950; S-902 to S-950; C-903 to S-950; S-904 to S-950; K-905 to S-950; T-906 to S-950; C-907 to S-950; G-908 to S-950; K-909 to S-950; G-910 to S-950; Y-911 to S-950; K-912 to S-950; K-913 to S-950; R-914 to S-950; S-915 to S-950; L-916 to S-950; K-917 to S-950; C-918 to S-950; L-919 to S-950; S-920 to S-950; H-921 to S-950; D-922 to S-950; G-923 to S-950; G-924 to S-950; V-925 to S-950; L-926 to S-950; S-927 to S-950; H-928 to S-950; E-929 to S-950; S-930 to S-950; C-931 to S-950; D-932 to S-950; P-933 to S-950; L-934 to S-950; K-935 to S-950; K-936 to S-950; P-937 to S-950; K-938 to S-950; H-939 to S-950; F-940 to S-950; I-941 to S-950; : D-942 to S-950; F-943 to S-950; C-944 to S-950; T-945 to S-950; of SEQ ID NO:2.

Moreover, C-terminal deletions of the METH1 polypeptide can also be described by the general formula 1−n₁, where n₁ is an integer from 2 to 950, where n corresponds to the position of amino acid residue identified in SEQ ID NO:2. Preferably, C-terminal deletions of the METH1 polypeptide of the invention shown as SEQ ID NO:2 include polypeptides comprising the amino acid sequence of residues: M-1 to C-949; M-1 to E-948; M-1 to A-947; M-1 to M-946; M-1 to T-945; M-1 to C-944; M-1 to F-943; M-1 to D-942; M-1 to I-941; M-1 to F-940; M-1 to H-939; M-1 to K-938; M-1 to P-937; M-1 to K-936; M-1 to K-935; M-1 to L-934; M-1 to P-933; M-1 to D-932; M-1 to C-931; M-1 to S-930; M-1 to E-929; M-1 to H-928; M-1 to S-927; M-1 to L-926; M-1 to V-925; M-1 to G-924; M-1 to G-923; M-1 to D-922; M-1 to H-921; M-1 to S-920; M-1 to L-919; M-1 to C-918; M-1 to K-917; M-1 to L-916; M-1 to S-915; M-1 to R-914; M-1 to K-913; M-1 to K-912; M-1 to Y-911; M-1 to G-910; M-1 to K-909; M-1 to G-908; M-1 to C-907; M-1 to T-906; M-1 to K-905; M-1 to S-904; M-1 to C-903; M-1 to S-902; M-1 to S-901; M-1 to W-900; M-1 to E-899; M-1 to G-898; M-1 to L-897; M-1 to Q-896; M-1 to W-895; M-1 to Q-894; M-1 to P-893; M-1 to C-892; M-1 to P-891; M-1 to H-890; M-1 to D-889; M-1 to A-888; M-1 to C-887; M-1 to P-886; M-1 to R-885; M-1 to T-884; M-1 to S-883; M-1 to A-882; M-1 to P-881; M-1 to K-880; M-1 to V-879; M-1 to E-878; M-1 to K-877; M-1 to A-876; M-1 to C-875; M-1 to E-874; M-1 to S-873; M-1 to A-872; M-1 to P-871; M-1 to Q-870; M-1 to G-869; M-1 to N-868; M-1 to I-867; M-1 to D-866; M-1 to R-865; M-1 to C-864; M-1 to E-863; M-1 to V-862; M-1 to L-861; M-1 to R-860; M-1 to R-859; M-1 to Q-858; M-1 to W-857; M-1 to G-856; M-1 to L-855; M-1 to E-854; M-1 to C-853; M-1 to S-852; M-1 to K-851; M-1 to S-850; M-1 to C-849; M-1 to E-848; M-1 to G-847; M-1 to W-846; M-1 to E-845; M-1 to E-844; M-1 to I-843; M-1 to V-842; M-1 to W-841; M-1 to A-840; M-1 to S-839; M-1 to F-838; M-1 to T-837; M-1 to P-836; M-1 to I-835; M-1 to A-834; M-1 to N-833; M-1 to F-832; M-1 to S-831; M-1 to E-830; M-1 to K-829; M-1 to K-828; M-1 to K-827; M-1 to K-826; M-1 to V-825; M-1 to F-824; M-1 to Y-823; M-1 to T-822; M-1 to Y-821; M-1 to K-820; M-1 to I-819; M-1 to K-818; M-1 to P-817; M-1 to R-816; M -1 to L-815; M-1 to A-814; M-1 to N-813; M-1 to G-812; M-1 to V-811; M-1 to T-810; M-1 to L-809; M-1 to V-808; M-1 to Q-807; M-1 to I-806; M-1 to T-805; M-1 to L-804; M-1 to P-803; M-1 to E-802; M-1 to K-801; M-1 to L-800; M-1 to P-799; M-1 to S-798; M-1 to F-797; M-1 to S-796; M-1 to R-795; M-1 to I-794; M-1 to R-793; M-1 to E-792; M-1 to L-791; M-1 to A-790; M-1 to A-789; M-1 to S-788; M-1 to S-787; M-1 to G-786; M-1 to S-785; M-1 to Y-784; M-1 to R-783; M-1 to L-782; M-1 to V-781; M-1 to V-780; M-1 to G-779; M-1 to K-778; M-1 to Y-777; M-1 to M-776; M-1 to I-775; M-1 to D-774; M-1 to Q-773; M-1 to E-772; M-1 to L-771; M-1 to T-770; M-1 to S-769; M-1 to L-768; M-1 to T-767; M-1 to Y-766; M-1 to D-765; M-1 to G-764; M-1 to N-763; M-1 to L-762; M-1 to I-761; M-1 to Y-760; M-1 to T-759; M-1 to G-758; M-1 to D-757; M-1 to A-756; M-1 to A-755; M-1 to K-754; M-1 to I-753; M-1 to A-752; M-1 to L-751; M-1 to F-750; M-1 to S-749; M-1 to G-748; M-1 to N-747; M-1 to N-746; M-1 to R-745; M-1 to S-744; M-1 to G-743; M-1 to R-742; M-1 to Q-741; M-1 to N-740; M-1 to R-739; M-1 to Q-738; M-1 to K-737; M-1 to V-736; M-1 to E-735; M-1 to I-734; M-1 to N-733; M-1 to T-732; M-1 to A-731; M-1 to G-730; M-1 to T-729; M-1 to P-728; M-1 to I-727; M-1 to T-726; M-1 to I-725; M-1 to I-724; M-1 to D-723; M-1 to H-722; M-1 to Y-721; M-1 to G-720; M-1 to P-719; M-1 to K-718; M-1 to A-717; M-1 to S-716; M-1 to T-715; M-1 to V-714; M-1 to S-713; M-1 to G-712; M-1 to S-711; M-1 to I-710; M-1 to K-709; M-1 to K-708; M-1 to C-707; M-1 to T-706; M-1 to S-705; M -1 to G-704; M-1 to N-703; M-1 to G-702; M -1 to G-701; M-1 to C-700; M-1 to V-699; M-1 to G-698; M-1 to C-697; M-1 to K-696; M-1 to D-695; M-1 to F-694; M-1 to K-693; M-1 to K-692; M-1 to K-691; M-1 to S-690; M-1 to D-689; M-1 to I-688; M-1 to I-687; M-1 to R-686; M-1 to D-685; M-1 to C-684; M-1 to G-683; M-1 to A-682; M-1 to K-681; M-1 to V-680; M-1 to C-679; M-1 to Q-678; M-1 to G-677; M-1 to Q-676; M-1 to V-675; M-1 to C-674; M-1 to V-673; M-1 to S-672; M-1 to T-671; M-1 to S-670; M-1 to D-669; M-1 to P-668; M-1 to S-667; M-1 to C-666; M-1 to P-665; M-1 to T-664; M-1 to G-663; M-1 to D-662; M-1 to V-661; M-1 to V-660; M-1 to K-659; M-1 to P-658; M-1 to Q-657; M-1 to L-656; M-1 to V-655; M-1 to F-654; M-1 to F-653; M-1 to Y-652; M-1 to G-651; M-1 to L-650; M-1 to G-649; M-1 to K-648; M-1 to A-647; M-1 to Q-646; M-1 to C-645; M-1 to I-644; M-1 to L-643; M-1 to K-642; M-1 to C-641; M-1 to R-640; M-1 to D-639; M-1 to K-638; M-1 to P-637; M-1 to S-636; M-1 to V-635; M-1 to G-634; M-1 to A-633; M-1 to Y-632; M-1 to K-631; M-1 to P-630; M-1 to I-629; M-1 to W-628; M-1 to E-627; M-1 to V-626; M-1 to A-625; M-1 to P-624; M-1 to G-623; M-1 to S-622; M-1 to G-621; M-1 to F-620; M-1 to S-619; M-1 to A-618; M-1 to K-617; M-1 to S-616; M-1 to F-615; M-1 to E-614; M-1 to N-613; M-1 to H-612; M-1 to A-611; M-1 to E-610; M-1 to C-609; M-1 to Q-608; M-1 to E-607; M-1 to E-606; M-1 to R-605; M-1 to F-604; M-1 to T-603; M-1 to K-602; M-1 to G-601; M-1 to N-600; M-1 to N-599; M-1 to D-598; M-1 to P-597; M-1 to C-596; M-1 to D-595; M-1 to E-594; M-1 to L-593; M-1 to N-592; M-1 to C-591; M-1 to S-590; M-1 to R-589; M-1 to Y-588; M-1 to R-587; M-1 to V-586; M-1 to R-585; M-1 to K-584; M-1 to G-583; M-1 to E-582; M-1 to C-581; M-1 to Y-580; M-1 to K-579; M-1 to G-578; M-1 to G-577; M-1 to N-576; M-1 to K-575; M-1 to P-574; M-1 to V-573; M-1 to P-572; M-1 to N-571; M-1 to D-570; M-1 to C-569; M-1 to E-568; M-1 to R-567; M-1 to M-566; M-1 to T-565; M-1 to Y-564; M-1 to Q-563; M-1 to V-562; M-1 to G-561; M-1 to G-560; M-1 to G-559; M-1 to C-558; M-1 to T-557; M-1 to R-556; M-1 to S-555; M-1 to C-554; M-1 to D-553; M-1 to G-552; M-1 to W-551; M-1 to P-550; M-1 to G-549; M-1 to W-548; M-1 to M-547; M-1 to G-546; M-1 to W-545; M-1 to S-544; M-1 to G-543; M-1 to H-542; M-1 to F-541; M-1 to P-540; M-1 to T-539; M-1 to D-538; M-1 to F-537; M-1 to H-536; M-1 to K-535; M-1 to R-534; M-1 to D-533; M-1 to T-532; M-1 to K-531; M-1 to N-530; M-1 to V-529; M-1 to C-528; M-1 to K-527; M-1 to G-526; M-1 to N-525; M-1 to I-524; M-1 to C-523; M-1 to W-522; M-1 to K-521; M-1 to G-520; M-1 to E-519; M-1 to G-518; M-1 to C-517; M-1 to S-516; M-1 to T-515; M-1 to G-514; M-1 to D-513; M-1 to A-512; M-1 to W-511; M-1 to P-510; M-1 to F-509; M-1 to H-508; M-1 to K-507; M-1 to T-506; M-1 to Q-505; M-1 to C-504; M-1 to V-503; M-1 to L-502; M-1 to V-501; M-1 to G-500; M-1 to G-499; M-1 to S-498; M-1 to T-497; M-1 to G-496; M-1 to T-495; M-1 to C-494; M-1 to W-493; M-1 to L-492; M-1 to T-491; M-1 to S-490; M-1 to C-489; M-1 to T-488; M-1 to S-487; M-1 to A-486; M-1 to A-485; M-1 to D-484; M-1 to P-483; M-1 to C-482; M-1 to H-481; M-1 to K-480; M-1 to S-479; M-1 to D-478; M-1 to E-477; M-1 to G-476; M-1 to F-475; M-1 to T-474; M-1 to F-473; M-1 to Q-472; M-1 to C-471; M-1 to Q-470; M-1 to R-469; M-1 to N-468; M-1 to A-467; M-1 to D-466; M-1 to Y-465; M-1 to S-464; M-1 to T-463; M-1 to G-462; M-1 to P-461; M-1 to L-460; M-1 to D-459; M-1 to G-458; M-1 to P-457; M-1 to L-456; M-1 to Q-455; M-1 to I-454; M-1 to P-453; M-1 to N-452; M-1 to Q-451; M-1 to P-450; M-1 to K-449; M-1 to D-448; M-1 to M-447; M-1 to L-446; M-1 to C-445; M-1 to E-444; M-1 to G-443; M-1 to H-442; M-1 to G-441; M-1 to N-440; M-1 to D-439; M-1 to L-438; M-1 to F-437; M-1 to S-436; M-1 to T-435; M-1 to I-434; M-1 to M-433; M-1 to Y-432; M-1 to A-431; M-1 to S-430; M-1 to C-429; M-1 to P-428; M-1 to S-427; M-1 to W-426; M-1 to P-425; M-1 to Q-424; M-1 to S-423; M-1 to H-422; M-1 to D-421; M-1 to L-420; M-1 to N-419; M-1 to S-418; M-1 to L-417; M-1 to M-416; M-1 to S-415; M-1 to A-414; M-1 to M-413; M-1 to M-412; M-1 to H-411; M-1 to S-410; M-1 to D-409; M-1 to Q-408; M-1 to N-407; M-1 to V-406; M-1 to G-405; M-1 to N-404; M-1 to L-403; M-1 to S-402; M-1 to A-401; M-1 to C-400; M-1 to Q-399; M-1 to K-398; M-1 to A-397; M-1 to D-396; M-1 to D-395; M-1 to H-394; M-1 to P-393; M-1 to M-392; M-1 to N-391; M-1 to F-390; M-1 to V-389; M-1 to H-388; M-1 to G-387; M-1 to L-386; M-1 to E-385; M-1 to H-384; M-1 to A-383; M-1 to T-382; M-1 to T-381; M-1 to F-380; M-1 to A-379; M-1 to A-378; M-1 to Q-377; M-1 to L-376; M-1 to G-375; M-1 to D-374; M-1 to D-373; M-1 to E-372; M-1 to I-371; M-1 to V-370; M-1 to S-369; M-1 to C-368; M-1 to S-367; M-1 to R-366; M-1 to S-365; M-1 to P-364; M-1 to D-363; M-1 to C-362; M-1 to V-361; M-1 to T-360; M-1 to G-359; M-1 to V-358; M-1 to D-357; M-1 to A-356; M-1 to M-355; M-1 to G-354; M-1 to L-353; M-1 to T-352; M-1 to D-351; M-1 to C-350; M-1 to T-349; M-1 to Q-348; M-1 to S-347; M-1 to G-346; M-1 to C-345; M-1 to L-344; M-1 to D-343; M-1 to Q-342; M-1 to R-341; M-1 to T-340; M-1 to F-339; M-1 to L-338; M-1 to I-337; M-1 to A-336; M-1 to T-335; M-1 to D-334; M-1 to Y-333; M-1 to H-332; M-1 to E-331; M-1 to A-330; M-1 to D-329; M-1 to R-328; M-1 to D-327; M-1 to S-326; M-1 to P-325; M-1 to P-324; M-1 to N-323; M-1 to H-322; M-1 to Q-321; M-1 to K-320; M-1 to Q-319; M-1 to W-318; M-1 to N-317; M-1 to C-316; M-1 to F-315; M-1 to N-314; M-1 to R-313; M-1 to L-312; M-1 to T-311; M-1 to L-310; M-1 to A-309; M-1 to A-308; M-1 to N-307; M-1 to S-306; M-1 to T-305; M-1 to V-304; M-1 to E-303; M-1 to P-302; M-1 to G-301; M-1 to K-300; M-1 to Q-299; M-1 to E-298; M-1 to D-297; M-1 to H-296; M-1 to I-295; M-1 to V-294; M-1 to L-293; M-1 to I-292; M-1 to K-291; M-1 to V-290; M-1 to V-289; M-1 to V-288; M-1 to L-287; M-1 to S-286; M-1 to V-285; M-1 to S-284; M-1 to N-283; M-1 to R-282; M-1 to L-281; M-1 to S-280; M-1 to P-279; M-1 to H-278; M-1 to K-277; M-1 to Y-276; M-1 to L-275; M-1 to R-274; M-1 to A-273; M-1 to A-272; M-1 to V-271; M-1 to S-270; M-1 to F-269; M-1 to L-268; M-1 to T-267; M-1 to L-266; M-1 to L-265; M-1 to Y-264; M-1 to H-263; M-1 to K-262; M-1 to L-261; M-1 to G-260; M-1 to S-259; M-1 to G-258; M-1 to H-257; M-1 to F-256; M-1 to E-255; M-1 to A-254; M-1 to M-253; M-1 to S-252; M-1 to Q-251; M-1 to D-250; M-1 to A-249; M-1 to V-248; M-1 to L-247; M-1 to M-246; M-1 to T-245; M-1 to E-244; M-1 to V-243; M-1 to Y-242; M-1 to R-241; M-1 to H-240; M-1 to S-239; M-1 to S-238; M-1 to V-237; M-1 to F-236; M-1 to R-235; M-1 to K-234; M-1 to K-233; M-1 to R-232; M-1 to I-231; M-1 to S-230; M-1 to G-229; M-1 to T-228; M-1 to G-227; M-1 to T-226; M-1 to P-225; M-1 to Q-224; M-1 to G-223; M-1 to V-222; M-1 to G-221; M-1 to Q-220; M-1 to L-219; M -1 to A-218; M -1 to P-217; M -1 to D-216; M -1 to Q-215; M -1 to P-214; M-1 to S-213; M-1 to W-212; M-1 to Q-211; M-1 to P-210; M-1 to G-209; M-1 to E-208; M-1 to D-207; M-1 to E-206; M-1 to G-205; M-1 to E-204; M -1 to T-203; M -1 to G-202; M -1 to E-201; M -1 to D-200; M -1 to E-199; M -1 to D-198; M-1 to E-197; M -1 to T-196; M -1 to E-195; M-1 to A-194; M-1 to K-193; M-1 to G-192; M-1 to T-191; M-1 to P-190; M-1 to R-189; M-1 to P-188; M-1 to E-187; M-1 to D-186; M-1 to D-185; M-1 to V-184; M-1 to V-183; M-1 to G-182; M-1 to C-181; M-1 to T-180; M-1 to G-179; M-1 to G-178; M-1 to V-177; M-1 to D-176; M-1 to G-175; M-1 to Q-174; M-1 to R-173; M-1 to N-172; M-1 to R-171; M-1 to R-170; M-1 to L-169; M-1 to L-168; M-1 to H-167; M-1 to F-166; M-1 to Q-165; M-1 to L-164; M-1 to P-163; M-1 to A-162; M-1 to P-161; M-1 to P-160; M-1 to K-159; M-1 to E-158; M-1 to G-157; M-1 to P-156; M-1 to A-155; M-1 to A-154; M-1 to T-153; M-1 to A-152; M-1 to L-151; M-1 to R-150; M-1 to E-149; M-1 to S-148; M-1 to A-147; M-1 to A-146; M-1 to P-145; M-1 to L-144; M-1 to P-143; M-1 to Q-142; M-1 to I-141; M-1 to F-140; M-1 to Y-139; M-1 to A-138; M-1 to E-137; M-1 to G-136; M-1 to L-135; M-1 to L-134; M-1 to Y-133; M-1 to F-132; M-1 to A-131; M-1 to G-130; M-1 to R-129; M-1 to V-128; M-1 to G-127; M-1 to E-126; M-1 to C-125; M-1 to L-124; M-1 to S-123; M-1 to L-122; M-1 to A-121; M-1 to A-120; M-1 to A-119; M-1 to S-118; M-1 to S-117; M-1 to P-116; M-1 to D-115; M-1 to G-114; M-1 to N-113; M-1 to V-112; M-1 to T-111; M-1 to G-110; M-1 to S-109; M-1 to Y-108; M-1 to F-107; M-1 to C-106; M-1 to H-105; M-1 to A-104; M-1 to L-103; M-1 to D-102; M-1 to T-101; M-1 to E-100; M-1 to P-99; M-1 to L-98; M-1 to P-97; M-1 to T-96; M-1 to E-95; M-1 to S-94; M-1 to G-93; M-1 to S-92; M-1 to K-91; M-1 to R-90; M-1 to G-89; M-1 to V-88; M-1 to N-87; M-1 to Q-86; M-1 to L-85; M-1 to T-84; M-1 to F-83; M-1 to G-82; M-1 to P-81; M-1 to A-80; M-1 to L-79; M-1 to F-78; M-1 to S-77; M-1 to S-76; M-1 to D-75; M-1 to P-74; M-1 to R-73; M-1 to L-72; M-1 to E-71; M-1 to L-70; M-1 to D-69; M-1 to L-68; M-1 to Q-67; M-1 to Q-66; M-1 to D-65; M-1 to F-64; M-1 to A-63; M-1 to H-62; M-1 to L-61; M-1 to R-60; M-1 to L-59; M-1 to R-58; M-1 to T-57; M-1 to T-56; M-1 to G-55; M-1 to H-54; M-1 to G-53; M-1 to P-52; M-1 to A-51; M-1 to R-50; M-1 to E-49; M-1 to L-48; M-1 to E-47; M-1 to P-46; M-1 to V-45; M-1 to V-44; M-1 to L-43; M-1 to E-42; M-1 to E-41; M-1 to D-40; M-1 to E-39; M-1 to E-38; M-1 to S-37; M-1 to P-36; M-1 to R-35; M-1 to G-34; M-1 to L-33; M-1 to A-32; M-1 to D-31; M-1 to S-30; M-1 to V-29; M-1 to A-28; M-1 to L-27; M-1 to L-26; M-1 to A-25; M-1 to A-24; M-1 to A-23; M-1 to L-22; M-1 to L-21; M-1 to L-20; M-1 to L-19; M-1 to T-18; M-1 to P-17; M-1 to V-16; M-1 to P-15; M-1 to G-14; M-1 to F-13; M-1 to S-12; M-1 to R-11; M-1 to S-10; M-1 to G-9; M-1 to P-8; M-1 to A-7; of SEQ ID NO:2. For example, any of the above listed N- or C-terminal deletions can be combined to produce a N- and C-terminal deleted METH1 polypeptide. Particularly preferred fragment of SEQ ID NO₂ are H542-Q894 and K801-S950.

Likewise, C-terminal deletions of the polypeptide of the invention shown as SEQ ID NO:2 include polypeptides comprising the amino acid sequence of residues: F-236 to S-950; F-236 to C-949; F-236 to E-948; F-236 to A-947; F-236 to M-946; F-236 to T- 945; F-236 to C-944; F-236 to F-943; F-236 to D-942; F-236 to I-941; F-236 to F-940; F-236 to H-939; F-236 to K-938; F-236 to P-937; F-236 to K-936; F-236 to K-935; F-236 to L-934; F-236 to P-933; F-236 to D-932; F-236 to C-931; F-236 to S-930; F-236 to E-929; F-236 to H-928; F-236 to S-927; F-236 to L-926; F-236 to V-925; F-236 to G-924; F-236 to G-923; F-236 to D-922; F-236 to H-921; F-236 to S-920; F-236 to L-919; F-236 to C-918; F-236 to K-917; F-236 to L-916; F-236 to S-915; F-236 to R-914; F-236 to K-913; F-236 to K-912; F-236 to Y-911; F-236 to G-910; F-236 to K-909; F-236 to G-908; F-236 to C-907; F-236 to T-906; F-236 to K-905; F-236 to S-904; F-236 to C-903; F-236 to S-902; F-236 to S-901; F-236 to W-900; F-236 to E-899; F-236 to G-898; F-236 to L-897; F-236 to Q-896; F-236 to W-895; F-236 to Q-894; F-236 to P-893; F-236 to C-892; F-236 to P-891; F-236 to I-890; F-236 to D-889; F-236 to A-888; F-236 to C-887; F-236 to P-886; F-236 to R-885; F-236 to T-884; F-236 to S-883; F-236 to A-882; F-236 to P-881; F-236 to K-880; F-236 to V-879; F-236 to E-878; F-236 to K-877; F-236 to A-876; F-236 to C -875; F-236 to E-874; F-236 to S-873; F-236 to A-872; F-236 to P-871; F-236 to Q-870; F-236 to G-869; F-236 to N-868; F-236 to I-867; F-236 to D-866; F-236 to R-865; F-236 to C-864; F-236 to E-863; F-236 to V-862; F-236 to L-861; F-236 to R-860; F-236 to R-859; F-236 to Q-858; F-236 to W-857; F-236 to G-856; F-236 to L-855; F-236 to E-854; F-236 to C-853; F-236 to S-852; F-236 to K-851; F-236 to S-850; F-236 to C-849; F-236 to E-848; F-236 to G-847; F-236 to W-846; F-236 to E-845; F-236 to E-844; F-236 to C-843; F-236 to V-842; F-236 to W-841; F-236 to A-840; F-236 to S-839; F-236 to F-838; F-236 to T-837; F-236 to P-836; F-236 to I-835; F-236 to A-834; F-236 to N-833; F-236 to F-832; F-236 to S-831; F-236 to E-830; F-236 to K-829; F-236 to K-828; F-236 to K-827; F-236 to K-826; F-236 to V-825; F-236 to F-824; F-236 to Y-823; F-236 to T-822; F-236 to Y-821; F-236 to K-820; F-236 to I-819; F-236 to K-818; F-236 to P-817; F-236 to R-816; F-236 to L-815; F-236 to A-814; F-236 to N-813; F-236 to G-812; F-236 to V-811; F-236 to T-810; F-236 to L-809; F-236 to V-808; F-236 to Q-807; F-236 to I-806; F-236 to T-805; F-236 to L-804; F-236 to P-803; F-236 to E-802; F-236 to K-801; F-236 to L-800; F-236 to P-799; F-236 to S-798; F-236 to F-797; F-236 to S-796; F-236 to R-795; F-236 to I-794; F-236 to R-793; F-236 to E-792; F-236 to L-791; F-236 to A-790; F-236 to A-789; F-236 to S-788; F-236 to S-787; F-236 to G-786; F-236 to S-785; F-236 to Y-784; F-236 to R-783; F-236 to L-782; F-236 to V-781; F-236 to V-780; F-236 to G-779; F-236 to K-778; F-236 to Y-777; F-236 to M-776; F-236 to I-775; F-236 to D-774; F-236 to Q-773; F-236 to E-772; F-236 to L-771; F-236 to T-770; F-236 to S-769; F-236 to L-768; F-236 to T-767; F-236 to Y-766; F-236 to D-765; F-236 to G-764; F-236 to N-763; F-236 to L-762; F-236 to I-761; F-236 to Y-760; F-236 to T-759; F-236 to G-758; F-236 to D-757; F-236 to A-756; F-236 to A-755; F-236 to K-754; F-236 to I-753; F-236 to A-752; F-236 to L-751; F-236 to F-750; F-236 to S-749; F-236 to G-748; F-236 to N-747; F-236 to N-746; F-236 to R-745; F-236 to S-744; F-236 to G-743; F-236 to R-742; F-236 to Q-741; F-236 to N-740; F-236 to R-739; F-236 to Q-738; F-236 to K-737; F-236 to V-736; F-236 to E-735; F-236 to I-734; F-236 to N-733; F-236 to T-732; F-236 to A-731; F-236 to G-730; F-236 to T-729; F-236 to P-728; F-236 to I-727; F-236 to T-726; F-236 to I-725; F-236 to I-724; F-236 to D-723; F-236 to H-722; F-236 to Y-721; F-236 to G-720; F-236 to P-719; F-236 to K-718; F-236 to A-717; F-236 to S-716; F-236 to T-715; F-236 to V-714; F-236 to S-713; F-236 to G-712; F-236 to S-711; F-236 to I-710; F-236 to K-709; F-236 to K-708; F-236 to C-707; F-236 to T-706; F-236 to S-705; F-236 to G-704; F-236 to N-703; F-236 to G-702; F-236 to G-701; F-236 to C-700; F-236 to V-699; F-236 to G-698; F-236 to C-697; F-236 to K-696; F-236 to D-695; F-236 to F-694; F-236 to K-693; F-236 to K-692; F-236 to K-691; F-236 to S-690; F-236 to D-689; F-236 to I-688; F-236 to I-687; F-236 to R-686; F-236 to D-685; F-236 to C-684; F-236 to G-683; F-236 to A-682; F-236 to K-681; F-236 to V-680; F-236 to C-679; F-236 to Q-678; F-236 to G-677; F-236 to Q-676; F-236 to V-675; F-236 to C-674; F-236 to V-673; F-236 to S-672; F-236 to T-671; F-236 to S-670; F-236 to D-669; F-236 to P-668; F-236 to S-667; F-236 to C-666; F-236 to P-665; F-236 to T-664; F-236 to G-663; F-236 to D-662; F-236 to V-661; F-236 to V-660; F-236 to K-659; F-236 to P-658; F-236 to Q-657; F-236 to L-656; F-236 to V-655; F-236 to F-654; F-236 to F-653; F-236 to Y-652; F-236 to G-651; F-236 to I-650; F-236 to G-649; F-236 to K-648; F-236 to A-647; F-236 to Q-646; F-236 to C-645; F-236 to I-644; F-236 to L-643; F-236 to K-642; F-236 to C-641; F-236 to R-640; F-236 to D-639; F-236 to K-638; F-236 to P-637; F-236 to S-636; F-236 to V-635; F-236 to G-634; F-236 to A-633; F-236 to Y-632; F-236 to K-631; F-236 to P-630; F-236 to I-629; F-236 to W-628; F-236 to E-627; F-236 to V-626; F-236 to A-625; F-236 to P-624; F-236 to G-623; F-236 to S-622; F-236 to G-621; F-236 to F-620; F-236 to S-619; F-236 to A-618; F-236 to K-617; F-236 to S-616; F-236 to F-615; F-236 to E-614; F-236 to N-613; F-236 to H-612; F-236 to A-611; F-236 to E-610; F-236 to C-609; F-236 to Q-608; F-236 to E-607; F-236 to E-606; F-236 to R-605; F-236 to F-604; F-236 to T-603; F-236 to K-602; F-236 to G-601; F-236 to N-600; F-236 to N-599; F-236 to D-598; F-236 to P-597; F-236 to C-596; F-236 to D-595; F-236 to E-594; F-236 to L-593; F-236 to N-592; F-236 to C-591; F-236 to S-590; F-236 to R-589; F-236 to Y-588; F-236 to R-587; F-236 to V-586; F-236 to R-585; F-236 to K-584; F-236 to G-583; F-236 to E-582; F-236 to C-581; F-236 to Y-580; F-236 to K-579; F-236 to G-578; F-236 to G-577; F-236 to N-576; F-236 to K-575; F-236 to P-574; F-236 to V-573; F-236 to P-572; F-236 to N-571; F-236 to D-570; F-236 to C-569; F-236 to E-568; F-236 to R-567; F-236 to M-566; F-236 to T-565; F-236 to Y-564; F-236 to Q-563; F-236 to V-562; F-236 to G-561; F-236 to G-560; F-236 to G-559; F-236 to C-558; F-236 to T-557; F-236 to R-556; F-236 to S-555; F-236 to C-554; F-236 to D-553; F-236 to G-552; F-236 to W-551; F-236 to P-550; F-236 to G-549; F-236 to W-548; F-236 to M-547; F-236 to G-546; F-236 to W-545; F-236 to G-544; F-236 to W-543; F-236 to M-542; F-236 to F-541; F-236 to P-540; F-236 to T-539; F-236 to D-538; F-236 to F-537; F-236 to H-536; F-236 to K-535; F-236 to R-534; F-236 to D-533; F-236 to T-532; F-236 to K-531; F-236 to N-530; F-236 to V-529; F-236 to C-528; F-236 to K-527; F-236 to G-526; F-236 to N-525; F-236 to I-524; F-236 to C-523; F-236 to W-522; F-236 to K-521; F-236 to G-520; F-236 to E-519; F-236 to G-518; F-236 to C-517; F-236 to S-516; F-236 to T-515; F-236 to G-514; F-236 to D-513; F-236 to A-512; F-236 to W-511; F-236 to P-510; F-236 to F-509; F-236 to H-508; F-236 to K-507; F-236 to T-506; F-236 to Q-505; F-236 to C-504; F-236 to V-503; F-236 to L-502; F-236 to V-501; F-236 to G-500; F-236 to G-499; F-236 to S-498; F-236 to T-497; F-236 to G-496; F-236 to T-495; F-236 to C-494; F-236 to W-493; F-236 to L-492; F-236 to T-491; F-236 to S-490; F-236 to C-489; F-236 to T-488; F-236 to S-487; F-236 to A-486; F-236 to A-485; F-236 to D-484; F-236 to P-483; F-236 to C-482; F-236 to H-481; F-236 to K-480; F-236 to S-479; F-236 to D-478; F-236 to E-477; F-236 to G-476; F-236 to F-475; F-236 to T-474; F-236 to F-473; F-236 to Q-472; F-236 to C-471; F-236 to Q-470; F-236 to R-469; F-236 to N-468; F-236 to A-467; F-236 to D-466; F-236 to Y-465; F-236 to S-464; F-236 to T-463; F-236 to G-462; F-236 to P-461; F-236 to L-460; F-236 to D-459; F-236 to G-458; F-236 to P-457; F-236 to L-456; F-236 to Q-455; F-236 to I-454; F-236 to P-453; F-236 to N-452; F-236 to Q-451; F-236 to P-450; F-236 to K-449; F-236 to D-448; F-236 to M-447; F-236 to L-446; F-236 to C-445; F-236 to E-444; F-236 to G-443; F-236 to H-442; F-236 to G-441; F-236 to N-440; F-236 to D-439; F-236 to L-438; F-236 to F-437; F-236 to S-436; F-236 to T-435; F-236 to I-434; F-236 to M-433; F-236 to Y-432; F-236 to A-431; F-236 to S-430; F-236 to C-429; F-236 to P-428; F-236 to S-427; F-236 to W-426; F-236 to P-425; F-236 to Q-424; F-236 to S-423; F-236 to H-422; F-236 to D-421; F-236 to L-420; F-236 to N-419; F-236 to S-418; F-236 to L-417; F-236 to M-416; F-236 to S-415; F-236 to A-414; F-236 to M-413; F-236 to M-412; F-236 to H-411; F-236 to S-410; F-236 to D-409; F-236 to Q-408; F-236 to N-407; F-236 to V-406; F-236 to G-405; F-236 to N-404; F-236 to L-403; F-236 to S-402; F-236 to A-401; F-236 to C-400; F-236 to Q-399; F-236 to K-398; F-236 to A-397; F-236 to D-396; F-236 to D-395; F-236 to H-394; F-236 to P-393; F-236 to M-392; F-236 to N-391; F-236 to F-390; F-236 to V-389; F-236 to H-388; F-236 to G-387; F-236 to L-386; F-236 to E-385; F-236 to H-384; F-236 to A-383; F-236 to T-382; F-236 to T-381; F-236 to F-380; F-236 to A-379; F-236 to A-378; F-236 to Q-377; F-236 to L-376; F-236 to G-375; F-236 to D-374; F-236 to D-373; F-236 to E-372; F-236 to I-371; F-236 to V-370; F-236 to S-369; F-236 to C-368; F-236 to S-367; F-236 to R-366; F-236 to S-365; F-236 to P-364; F-236 to D-363; F-236 to C-362; F-236 to V-361; F-236 to T-360; F-236 to G-359; F-236 to V-358; F-236 to D-357; F-236 to A-356; F-236 to M-355; F-236 to G-354; F-236 to L-353; F-236 to T-352; F-236 to D-351; F-236 to C-350; F-236 to T-349; F-236 to Q-348; F-236 to S-347; F-236 to G-346; F-236 to C-345; F-236 to L-344; F-236 to D-343; F-236 to Q-342; F-236 to R-341; F-236 to T-340; F-236 to F-339; F-236 to L-338; F-236 to I-337; F-236 to A-336; F-236 to T-335; F-236 to D-334; F-236 to Y-333; F-236 to H-332; F-236 to E-331; F-236 to A-330; F-236 to D-329; F-236 to R-328; F-236 to D-327; F-236 to S-326; F-236 to P-325; F-236 to P-324; F-236 to N-323; F-236 to H-322; F-236 to Q-321; F-236 to K-320; F-236 to Q-319; F-236 to W-318; F-236 to N-317; F-236 to C-316; F-236 to F-315; F-236 to N-314; F-236 to R-313; F-236 to L-312; F-236 to T-311; F-236 to L-310; F-236 to A-309; F-236 to A-308; F-236 to N-307; F-236 to S-306; F-236 to T-305; F-236 to V-304; F-236 to E-303; F-236 to P-302; F-236 to G-301; F-236 to K-300; F-236 to Q-299; F-236 to E-298; F-236 to D-297; F-236 to H-296; F-236 to I-295; F-236 to V-294; F-236 to L-293; F-236 to I-292; F-236 to K-291; F-236 to V-290; F-236 to V-289; F-236 to V-288; F-236 to L-287; F-236 to S-286; F-236 to V-285; F-236 to S-284; F-236 to N-283; F-236 to R-282; F-236 to I-281; F-236 to S-280; F-236 to P-279; F-236 to H-278; F-236 to K-277; F-236 to Y-276; F-236 to L-275; F-236 to R-274; F-236 to A-273; F-236 to A-272; F-236 to V-271; F-236 to S-270; F-236 to F-269; F-236 to L-268; F-236 to T-267; F-236 to L-266; F-236 to L-265; F-236 to Y-264; F-236 to H-263; F-236 to K-262; F-236 to L-261; F-236 to G-260; F-236 to S-259; F-236 to −258; F-236 to H-257; F-236 to F-256; F-236 to E-255; F-236 to A-254; F-236 to M-253; F-236 to S-252; F-236 to Q-251; F-236 to D-250; F-236 to A-249; F-236 to V-248; F-236 to L-247; F-236 to M-246; F-236 to T-245; F-236 to E-244; F-236 to V-243; and/or F-236 to Y-242 of SEQ ID NO:2.

Likewise, C-terminal deletions of the polypeptide of the invention shown as SEQ ID NO:2 include polypeptides comprising the amino acid sequence of residues: L-33 to S-950; L-33 to C-949; L-33 to E-948; L-33 to A-947; L-33 to M-946; L-33 to T-945; L-33 to C-944; L-33 to F-943; L-33 to D-942; L-33 to I-941; L-33 to F-940; L-33 to H-939; L-33 to K-938; L-33 to P-937; L-33 to K-936; L-33 to K-935; L-33 to L-934; L-33 to P-933; L-33 to D-932; L-33 to C-931; L-33 to S-930; L-33 to E-929; L-33 to H-928; L-33 to S-927; L-33 to L-926; L-33 to V-925; L-33 to G-924; L-33 to G-923; L-33 to D-922; L-33 to S-921; L-33 to S-920; L-33 to L-919; L-33 to C-918; L-33 to K-917; L-33 to L-916; L-33 to S-915; L-33 to R-914; L-33 to K-913; L-33 to K-912; L-33 to Y-911; L-33 to G-910; L-33 to K-909; L-33 to R-908; L-33 to C-907; L-33 to T-906; L-33 to K-905; L-33 to G -904; L-33 to C-903; L-33 to S-902; L-33 to S-901; L-33 to W-900; L-33 to E-899; L-33 to −898; L-33 to L-897; L-33 to Q-896; L-33 to W-895; L-33 to Q-894; L-33 to P-893; L-33 to C-892; L-33 to P-891; L-33 to Q-890; L-33 to D-889; L-33 to A-888; L-33 to C-887; L-33 to P-886; L-33 to R-885; L-33 to T-884; L-33 to D-883; L-33 to A-882; L-33 to P-881; L-33 to K-880; L-33 to V-879; L-33 to E-878; L-33 to K-877; L-33 to A-876; L-33 to C-875; L-33 to E-874; L-33 to S-873; L-33 to A-872; L-33 to P-871; L-33 to Q-870; L-33 to G-869; L-33 to N-868; L-33 to I-867; L-33 to D-866; L-33 to R-865; L-33 to C-864; L-33 to E-863; L-33 to V-862; L-33 to L-861; L-33 to R-860; L-33 to R-859; L-33 to Q-858; L-33 to W-857; L-33 to G-856; L-33 to L-855; L-33 to E-854; L-33 to C-853; L-33 to S-852; L-33 to K-851; L-33 to S-850; L-33 to C-849; L-33 to E-848; L-33 to G-847; L-33 to W-846; L-33 to E-845; L-33 to E-844; L-33 to I-843; L-33 to V-842; L-33 to W-841; L-33 to A-840; L-33 to S-839; L-33 to F-838; L-33 to T-837; L-33 to P-836; L-33 to I-835; L-33 to A-834; L-33 to N-833; L-33 to F-832; L-33 to S-831; L-33 to E-830; L-33 to K-829; L-33 to K-828; L-33 to K-827; L-33 to K-826; L-33 to V-825; L-33 to F-824; L-33 to Y-823; L-33 to T-822; L-33 to Y-821; L-33 to K-820; L-33 to I-819; L-33 to K-818; L-33 to P-817; L-33 to R-816; L-33 to L-815; L-33 to A-814; L-33 to N-813; L-33 to G-812; L-33 to V-811; L-33 to T-810; L-33 to L-809; L-33 to V-808; L-33 to Q-807; L-33 to I-806; L-33 to T-805; L-33 to L-804; L-33 to P-803; L-33 to E-802; L-33 to K-801; L-33 to L-800; L-33 to P-799; L-33 to S-798; L-33 to F-797; L-33 to S-796; L-33 to R-795; L-33 to I-794; L-33 to R-793; L-33 to E-792; L-33 to L-791; L-33 to A-790; L-33 to A-789; L-33 to S-788; L-33 to S-787; L-33 to G-786; L-33 to S-785; L-33 to Y-784; L-33 to R-783; L-33 to L-782; L-33 to V-781; L-33 to V-780; L-33 to G-779; L-33 to K-778; L-33 to Y-777; L-33 to M-776; L-33 to I-775; L-33 to D-774; L-33 to Q-773; L-33 to E-772; L-33 to L-771; L-33 to T-770; L-33 to S-769; 33 to L-768; L-33 to T-767; L-33 to Y-766; L-33 to D-765; L-33 to G-764; L-33 to N-763; L-33 to L-762; L-33 to T-761; L-33 to Y-760; L-33 to T-759; L-33 to G-758; L-33 to D-757; L-33 to A-756; L-33 to A-755; L-33 to K-754; L-33 to I-753; L-33 to A-752; L-33 to L-751; L-33 to F-750; L-33 to S-749; L-33 to G-748; L-33 to N-747; L-33 to N-746; L-33 to R-745; L-33 to I-744; L-33 to G-743; L-33 to R-742; L-33 to Q-741; L-33 to N-740; L-33 to R-739; L-33 to Q-738; L-33 to K-737; L-33 to V-736; L-33 to E-735; L-33 to I-734; L-33 to N-733; L-33 to T-732; L-33 to A-731; L-33 to G-730; L-33 to T-729; L-33 to P-728; L-33 to I-727; L-33 to T-726; L-33 to I-725; L-33 to I-724; L-33 to D-723; L-33 to R-722; L-33 to Y-721; L-33 to G-720; L-33 to P-719; L-33 to K-718; L-33 to A-717; L-33 to S-716; L-33 to T-715; L-33 to V-714; L-33 to S-713; L-33 to G-712; L-33 to S-711; L-33 to I-710; L-33 to K-709; L-33 to K-708; L-33 to C-707; L-33 to T-706; L-33 to S-705; L-33 to G-704; L-33 to N-703; L-33 to G-702; L-33 to G-701; L-33 to C-700; L-33 to V-699; L-33 to G-698; L-33 to C-697; L-33 to K-696; L-33 to D-695; L-33 to F-694; L-33 to K-693; L-33 to K-692; L-33 to K-691; L-33 to S-690; L-33 to D-689; L-33 to I-688; L-33 to I-687; L-33 to R-686; L-33 to D-685; L-33 to C-684; L-33 to G-683; L-33 to A-682; L-33 to K-681; L-33 to V-680; L-33 to C-679; L-33 to Q-678; L-33 to G-677; L-33 to Q-676; L-33 to V-675; L-33 to C-674; L-33 to V-673; L-33 to S-672; L-33 to T-671; L-33 to S-670; L-33 to D-669; L-33 to P-668; L-33 to S-667; L-33 to C-666; L-33 to P-665; L-33 to T-664; L-33 to G-663; L-33 to D-662; L-33 to V-661; L-33 to V-660; L-33 to K-659; L-33 to P-658; L-33 to Q-657; L-33 to L-656; L-33 to V-655; L-33 to F-654; L-33 to F-653; L-33 to Y-652; L-33 to G-651; L-33 to I-650; L-33 to G-649; L-33 to K-648; L-33 to A-647; L-33 to Q-646; L-33 to C-645; L-33 to I-644; L-33 to L-643; L-33 to K-642; L-33 to C-641; L-33 to R-640; L-33 to D-639; L-33 to K-638; L-33 to P-637; L-33 to S-636; L-33 to V-635; L-33 to G-634; L-33 to A-633; L-33 to Y-632; L-33 to K-631; L-33 to P-630; L-33 to I-629; L-33 to W-628; L-33 to E-627; L-33 to V-626; L-33 to A-625; L-33 to P-624; L-33 to G-623; L-33 to S-622; L-33 to G-621; L-33 to F-620; L-33 to A -619; L-33 to A-618; L-33 to K-617; L-33 to S-616; L-33 to F-615; L-33 to E-614; L-33 to N-613; L-33 to H-612; L-33 to A-611; L-33 to E-610; L-33 to C-609; L-33 to Q-608; L-33 to E-607; L-33 to E-606; L-33 to R-605; L-33 to F-604; L-33 to T-603; L-33 to K-602; L-33 to G-601; L-33 to N-600; L-33 to N-599; L-33 to D-598; L-33 to P-597; L-33 to C-596; L-33 to D-595; L-33 to E-594; L-33 to L-593; L-33 to N-592; L-33 to C-591; L-33 to S-590; L-33 to R-589; L-33 to Y-588; L-33 to R-587; L-33 to V-586; L-33 to R-585; L-33 to K-584; L-33 to G-583; L-33 to E-582; L-33 to C-581; L-33 to Y-580; L-33 to K-579; L-33 to G-578; L-33 to G-577; L-33 to N-576; L-33 to K-575; L-33 to P-574; L-33 to V-573; L-33 to P-572; L-33 to N-571; L-33 to D-570; L-33 to C-569; L-33 to E-568; L-33 to R-567; L-33 to M-566; L-33 to T-565; L-33 to Y-564; L-33 to Q-563; L-33 to V-562; L-33 to G-561; L-33 to G-560; L-33 to G-559; L-33 to C-558; L-33 to T-557; L-33 to R-556; L-33 to S-555; L-33 to C-554; L-33 to D-553; L-33 to G-552; L-33 to W-551; L-33 to P-550; L-33 to G-549; L-33 to W-548; L-33 to M-547; L-33 to G-546; L-33 to W-545; L-33 to S-544; L-33 to G-543; L-33 to H-542; L-33 to F-541; L-33 to P-540; L-33 to T-539; L-33 to D-538; L-33 to F-537; L-33 to H-536; L-33 to K-535; L-33 to R-534; L-33 to D-533; L-33 to T-532; L-33 to K-531; L-33 to N-530; L-33 to V-529; L-33 to C-528; L-33 to K-527; L-33 to G-526; L-33 to N-525; L-33 to I-524; L-33 to C-523; L-33 to W-522; L-33 to K-521; L-33 to G-520; L-33 to E-519; L-33 to G-518; L-33 to C-517; L-33 to S-516; L-33 to T-515; L-33 to G-514; L-33 to D-513; L-33 to A-512; L-33 to W-511; L-33 to P-510; L-33 to F-509; L-33 to H-508; L-33 to K-507; L-33 to T-506; L-33 to Q-505; L-33 to C-504; L-33 to V-503; L-33 to L-502; L-33 to V-501; L-33 to G-500; L-33 to G-499; L-33 to S-498; L-33 to T-497; L-33 to G-496; L-33 to T-495; L-33 to C-494; L-33 to W-493; L-33 to L-492; L-33 to T-491; L-33 to S-490; L-33 to C-489; L-33 to T-488; L-33 to S-487; L-33 to A-486; L-33 to A-485; L-33 to D-484; L-33 to P-483; L-33 to C-482; L-33 to H-481; L-33 to K-480; L-33 to S-479; L-33 to D-478; L-33 to E-477; L-33 to G-476; L-33 to F-475; L-33 to T-474; L-33 to F-473; L-33 to Q-472; L-33 to C-471; L-33 to Q-470; L-33 to R-469; L-33 to N-468; L-33 to A-467; L-33 to D-466; L-33 to Y-465; L-33 to S-464; L-33 to T-463; L-33 to G-462; L-33 to P-461; L-33 to L-460; L-33 to D-459; L-33 to G-458; L-33 to P-457; L-33 to L-456; L-33 to Q-455; L-33 to I-454; L-33 to P-453; L-33 to N-452; L-33 to Q-451; L-33 to P-450; L-33 to K-449; L-33 to D-448; L-33 to M-447; L-33 to L-446; L-33 to C-445; L-33 to E-444; L-33 to G-443; L-33 to H-442; L-33 to G-441; L-33 to N-440; L-33 to D-439; L-33 to L-438; L-33 to F-437; L-33 to S-436; L-33 to T-435; L-33 to I-434; L-33 to M-433; L-33 to Y-432; L-33 to A-431; L-33 to S-430; L-33 to C-429; L-33 to P-428; L-33 to S-427; L-33 to W-426; L-33 to P-425; L-33 to Q-424; L-33 to S-423; L-33 to H-422; L-33 to D-421; L-33 to L-420; L-33 to N-419; L-33 to S-418; L-33 to L-417; L-33 to M-416; L-33 to S-415; L-33 to A-414; L-33 to M-413; L-33 to M-412; L-33 to H-411; L-33 to S-410; L-33 to D-409; L-33 to Q-408; L-33 to N-407; L-33 to V-406; L-33 to G-405; L-33 to N-404; L-33 to L-403; L-33 to S-402; L-33 to A-401; L-33 to C-400; L-33 to Q-399; L-33 to K-398; L-33 to A-397; L-33 to D-396; L-33 to D-395; L-33 to H-394; L-33 to P-393; L-33 to M-392; L-33 to N-391; L-33 to F-390; L-33 to V-389; L-33 to H-388; L-33 to G-387; L-33 to L-386; L-33 to E-385; L-33 to H-384; L-33 to A-383; L-33 to T-382; L-33 to T-381; L-33 to F-380; L-33 to A-379; L-33 to A-378; L-33 to Q-377; L-33 to L-376; L-33 to G-375; L-33 to D-374; L-33 to D-373; L-33 to E-372; L-33 to I-371; L-33 to V-370; L-33 to S-369; L-33 to C-368; L-33 to S-367; L-33 to R-366; L-33 to S-365; L-33 to P-364; L-33 to D-363; L-33 to C-362; L-33 to V-361; L-33 to T-360; L-33 to G-359; L-33 to V-358; L-33 to D-357; L-33 to A-356; L-33 to M-355; L-33 to G-354; L-33 to L-353; L-33 to T-352; L-33 to D-351; L-33 to C-350; L-33 to T-349; L-33 to Q-348; L-33 to S-347; L-33 to G-346; L-33 to C-345; L-33 to L-344; L-33 to D-343; L-33 to Q-342; L-33 to R-341; L-33 to T-340; L-33 to F-339; L-33 to L-338; L-33 to I-337; L-33 to A-336; L-33 to T-335; L-33 to D-334; L-33 to Y-333; L-33 to H-332; L-33 to E-331; L-33 to A-330; L-33 to D-329; L-33 to R-328; L-33 to D-327; L-33 to S-326; L-33 to P-325; L-33 to P-324; L-33 to N-323; L-33 to H-322; L-33 to Q-321; L-33 to K-320; L-33 to Q-319; L-33 to W-318; L-33 to N-317; L-33 to C-316; L-33 to F-315; L-33 to N-314; L-33 to R-313; L-33 to L-312; L-33 to T-311; L-33 to L-310; L-33 to A-309; L-33 to A-308; L-33 to N-307; L-33 to S-306; L-33 to T-305; L-33 to V-304; L-33 to E-303; L-33 to P-302; L-33 to G-301; L-33 to K-300; L-33 to Q-299; L-33 to E-298; L-33 to D-297; L-33 to H-296; L-33 to I-295; L-33 to V-294; L-33 to L-293; L-33 to I-292; L-33 to K-291; L-33 to V-290; L-33 to V-289; L-33 to V-288; L-33 to L-287; L-33 to S-286; L-33 to V-285; L-33 to S-284; L-33 to N-283; L-33 to R-282; L-33 to I-281; L-33 to S-280; L-33 to P-279; L-33 to H-278; L-33 to K-277; L-33 to Y-276; L-33 to L-275; L-33 to R-274; L-33 to A-273; L-33 to A-272; L-33 to V-271; L-33 to S-270; L-33 to F-269; L-33 to L-268; L-33 to T-267; L-33 to L-266; L-33 to L-265; L-33 to Y-264; L-33 to H-263; L-33 to K-262; L-33 to L-261; L-33 to G-260; L-33 to S-259; L-33 to G-258; L-33 to H-257; L-33 to F-256; L-33 to E-255; L-33 to A-254; L-33 to M-253; L-33 to S-252; L-33 to Q-251; L-33 to D-250; L-33 to A-249; L-33 to V-248; L-33 to L-247; L-33 to M-246; L-33 to T-245; L-33 to E-244; L-33 to V-243; L-33 to Y-242; L-33 to R-241; L-33 to H-240; L-33 to S-239; L-33 to S-238; L-33 to V-237; L-33 to F-236; L-33 to R-235; L-33 to K-234; L-33 to K-233; L-33 to R-232; L-33 to I-231; L-33 to S-230; L-33 to G-229; L-33 to T-228; L-33 to G-227; L-33 to T-226; L-33 to P-225; L-33 to Q-224; L-33 to G-223; L-33 to V-222; L-33 to G-221; L-33 to Q-220; L-33 to L-219; L-33 to A-218; L-33 to P-217; L-33 to D-216; L-33 to Q-215; L-33 to P-214; L-33 to S-213; L-33 to W-212; L-33 to Q-211; L-33 to P-210; L-33 to G-209; L-33 to E-208; L-33 to D-207; L-33 to E-206; L-33 to G-205; L-33 to E-204; L-33 to T-203; L-33 to G-202; L-33 to E-201; L-33 to D-200; L-33 to E-199; L-33 to D-198; L-33 to E-197; L-33 to T-196; L-33 to E-195; L-33 to A-194; L-33 to K-193; L-33 to G-192; L-33 to T-191; L-33 to P-190; L-33 to R-189; L-33 to P-188; L-33 to E-187; L-33 to D-186; L-33 to D-185; L-33 to V-184; L-33 to V-183; L-33 to G-182; L-33 to C-181; L-33 to T-180; L-33 to G-179; L-33 to G-178; L-33 to V-177; L-33 to D-176; L-33 to G-175; L-33 to Q-174; L-33 to R-173; L-33 to N-172; L-33 to R-171; L-33 to R-170; L-33 to L-169; L-33 to L-168; L-33 to H-167; L-33 to F-166; L-33 to Q-165; L-33 to L-164; L-33 to P-163; L-33 to A-162; L-33 to P-161; L-33 to P-160; L-33 to K-159; L-33 to E-158; L-33 to G-157; L-33 to P-156; L-33 to A-155; L-33 to A-154; L-33 to T-153; L-33 to A-152; L-33 to L-151; L-33 to R-150; L-33 to E-149; L-33 to S-148; L-33 to A-147; L-33 to A-146; L-33 to P-145; L-33 to L-144; L-33 to P-143; L-33 to Q-142; L-33 to I-141; L-33 to F-140; L-33 to Y-139; L-33 to A-138; L-33 to E-137; L-33 to G-136; L-33 to L-135; L-33 to L-134; L-33 to Y-133; L-33 to F-132; L-33 to A-131; L-33 to G-130; L-33 to R-129; L-33 to V-128; L-33 to G-127; L-33 to E-126; L-33 to C-125; L-33 to L-124; L-33 to S-123; L-33 to L-122; L-33 to A-121; L-33 to A-120; L-33 to A-119; L-33 to S-118; L-33 to S-117; L-33 to P-116; L-33 to D-115; L-33 to G-114; L-33 to N-113; L-33 to V-112; L-33 to T-111; L-33 to G-110; L-33 to S-109; L-33 to Y-108; L-33 to F-107; L-33 to C-106; L-33 to H-105; L-33 to A-104; L-33 to L-103; L-33 to D-102; L-33 to T-101; L-33 to E-100; L-33 to P-99; L-33 to L-98; L-33 to P-97; L-33 to T-96; L-33 to E-95; L-33 to S-94; L-33 to G-93; L-33 to S-92; L-33 to K-91; L-33 to R-90; L-33 to G-89; L-33 to V-88; L-33 to N-87; L-33 to Q-86; L-33 to L-85; L-33 to T-84; L-33 to F-83; L-33 to G-82; L-133 to P-81; L-33 to A-80; L-33 to L-79; L-33 to F-78; L-33 to S-77; L-33 to S-76; L-33 to D-75; L-33 to P-74; L-33 to R-73; L-33 to L-72; L-33 to E-71; L-33 to L-70; L-33 to D-69; L-33 to L-68; L-33 to Q-67; L-33 to Q-66; L-33 to D-65; L-33 to F-64; L-33 to A-63; L-33 to H-62; L-33 to L-61; L-33 to R-60; L-33 to L-59; L-33 to R-58; L-33 to T-57; L-33 to T-56; L-33 to G-55; L-33 to H-54; L-33 to G-53; L-33 to P-52; L-33 to A-51; L-33 to R-50; L-33 to E-49; L-33 to L-48; L-33 to E-47; L-33 to P-46; L-33 to V-45; L-33 to V-44; L-33 to L-43; L-33 to E-42; L-33 to E-41; L-33 to D-40; and/or L-33 to E-39 of SEQ ID NO:2.

Deletion mutants of METH1 may also be made which comprise all or part of the additional sequence described in SEQ ID NO: 126. For example, exemplary deletion mutants include: Q-2 to S-967; R-3 to S-967; A-4 to S-967; V-5 to S-967; P-6 to S-967; E-7 to S-967; G-8 to S-967; F-9 to S-967; G-10 to S-967; R-11 to S-976; R-12 to S-967; K-13 to S-967; L-14 to S-967; G-15 to S-967; S-16 to S-967; D-17 to S-967; and M-18 to S-967.

Moreover, N-terminal deletions of the METH2 polypeptide can be described by the general formula m₂-890, where m₂ is an integer from 2 to 889, where m corresponds to the position of the amino acid residue identified in SEQ ID NO:4. Preferably, N-terminal deletions of the METH2 polypeptide of the invention shown as SEQ ID NO:4 include polypeptides comprising the amino acid sequence of residues: F-2 to L-890; P-3 to L-890; A-4 to L-890; P-5 to L-890; A-6 to L-890; A-7 to L-890; P-8 to L-890; R-9 to L-890; W-10 to L-890; L-11 to L-890; P-12 to L-890; F-13 to L-890; L-14 to L-890; L-15 to L-890; L-16 to L-890; L-17 to L-890; L-18 to L-890; L-19 to L-890; L-20 to L-890; L-21 to L-890; L-22 to L-890; P-23 to L-890; L-24 to L-890; A-25 to L-890; R-26 to L-890; G-27 to L-890; A-28 to L-890; P-29 to L-890; A-30 to L-890; R-31 to L-890; P-32 to L-890; A-33 to L-890; A-34 to L-890; G-35 to L-890; G-36 to L-890; Q-37 to L-890; A-38 to L-890; S-39 to L-890; E-40 to L-890; L-41 to L-890; V-42 to L-890; V-43 to L-890; P-44 to L-890; T-45 to L-890; R-46 to L-890; L-47 to L-890; P-48 to L-890; G-49 to L-890; S-50 to L-890; A-51 to L-890; G-52 to L-890; E-53 to L-890; L-54 to L-890; A-55 to L-890; L-56 to L-890; H-57 to L-890; L-58 to L-890; S-59 to L-890; A-60 to L-890; F-61 to L-890; G-62 to L-890; K-63 to L-890; G-64 to L-890; F-65 to L-890; V-66 to L-890; L-67 to L-890; R-68 to L-890; L-69 to L-890; A-70 to L-890; P-71 to L-890; D-72 to L-890; D-73 to L-890; S-74 to L-890; F-75 to L-890; L-76 to L-890; A-77 to L-890; P-78 to L-890; E-79 to L-890; F-80 to L-890; K-81 to L-890; I-82 to L-890; E-83 to L-890; R-84 to L-890; L-85 to L-890; G-86 to L-890; G-87 to L-890; S-88 to L-890; G-89 to L-890; R-90 to L-890; A-91 to L-890; T-92 to L-890; G-93 to L-890; G-94 to L-890; E-95 to L-890; R-96 to L-890; G-97 to L-890; L-98 to L-890; R-99 to L-890; G-100 to L-890; C-101 to L-890; F-102 to L-890; F-103 to L-890; S-104 to L-890; G-105 to L-890; T-106 to L-890; V-107 to L-890; N-108 to L-890; G-109 to L-890; E-110 to L-890; P-111 to L-890; E-112 to L-890; S-113 to L-890; L-114 to L-890; A-115 to L-890; A-116 to L-890; V-117 to L-890; S-118 to L-890; L-119 to L-890; C-120 to L-890; R-121 to L-890; G-122 to L-890; L-123 to L-890; S-124 to L-890; G-125 to L-890; S-126 to L-890; F-127 to L-890; L-128 to L-890; L-129 to L-890; D-130 to L-890; G-131 to L-890; E-132 to L-890; E-133 to L-890; F-134 to L-890; T-135 to L-890; I-136 to L-890; Q-137 to L-890; P-138 to L-890; Q-139 to L-890; G-140 to L-890; A-141 to L-890; G-142 to L-890; G-143 to L-890; S-144 to L-890; L-145 to L-890; A-146 to L-890; Q-147 to L-890; P-148 to L-890; H-149 to L-890; R-150 to L-890; L-151 to L-890; Q-152 to L-890; R-153 to L-890; W-154 to L-890; G-155 to L-890; P-156 to L-890; A-157 to L-890; G-158 to L-890; A-159 to L-890; R-160 to L-890; P-161 to L-890; L-162 to L-890; P-163 to L-890; R-164 to L-890; G-165 to L-890; P-166 to L-890; E-167 to L-890; W-168 to L-890; E-169 to L-890; V-170 to L-890; E-171 to L-890; T-172 to L-890; G-173 to L-890; E-174 to L-890; G-175 to L-890; Q-176 to L-890; R-177 to L-890; Q-178 to L-890; E-179 to L-890; R-180 to L-890; G-181 to L-890; D-182 to L-890; H-183 to L-890; Q-184 to L-890; E-185 to L-890; D-186 to L-890; S-187 to L-890; E-188 to L-890; E-189 to L-890; E-190 to L-890; S-191 to L-890; Q-192 to L-890; E-193 to L-890; E-194 to L-890; E-195 to L-890; A-196 to L-890; E-197 to L-890; G-198 to L-890; A-199 to L-890; S-200 to L-890; E-201 to L-890; P-202 to L-890; P-203 to L-890; P-204 to L-890; P-205 to L-890; L-206 to L-890; G-207 to L-890; A-208 to L-890; T-209 to L-890; S-210 to L-890; R-211 to L-890; T-212 to L-890; K-213 to L-890; R-214 to L-890; F-215 to L-890; V-216 to L-890; S-217 to L-890; E-218 to L-890; A-219 to L-890; R-220 to L-890; F-221 to L-890; V-222 to L-890; E-223 to L-890; T-224 to L-890; L-225 to L-890; L-226 to L-890; V-227 to L-890; A-228 to L-890; D-229 to L-890; A-230 to L-890; S-231 to L-890; M-232 to L-890; A-233 to L-890; A-234 to L-890; F-235 to L-890; Y-236 to L-890; G-237 to L-890; A-238 to L-890; D-239 to L-890; L-240 to L-890; Q-241 to L-890; N-242 to L-890; H-243 to L-890; I-244 to L-890; L-245 to L-890; T -246 to L-890; L-247 to L-890; M -248 to L-890; S-249 to L-890; V-250 to L-890; A-251 to L-890; A-252 to L-890; R-253 to L-890; I-254 to L-890; Y-255 to L-890; K-256 to L-890; H-257 to L-890; P-258 to L-890; S-259 to L-890; I-260 to L-890; K-261 to L-890; N-262 to L-890; S-263 to L-890; I-264 to L-890; N-265 to L-890; L-266 to L-890; M-267 to L-890; V-268 to L-890; V-269 to L-890; K-270 to L-890; V-271 to L-890; L-272 to L-890; I-273 to L-890; V-274 to L-890; E-275 to L-890; D-276 to L-890; E-277 to L-890; K-278 to L-890; W-279 to L-890; G-280 to L-890; P-281 to L-890; E-282 to L-890; V-283 to L-890; S-284 to L-890; D-285 to L-890; N-286 to L-890; G-287 to L-890; G-288 to L-890; L-289 to L-890; T-290 to L-890; L-291 to L-890; R-292 to L-890; N-293 to L-890; F-294 to L-890; C-295 to L-890; N-296 to L-890; W-297 to L-890; Q-298 to L-890; R-299 to L-890; R-300 to L-890; F-301 to L-890; N-302 to L-890; Q-303 to L-890; P-304 to L-890; S-305 to L-890; D-306 to L-890; R-307 to L-890; H-308 to L-890; P-309 to L-890; E-310 to L-890; H-311 to L-890; Y-312 to L-890; D-313 to L-890; T-314 to L-890; A-315 to L-890; I-316 to L-890; L-317 to L-890; L-318 to L-890; T-319 to L-890; R-320 to L-890; Q-321 to L-890; N-322 to L-890; F-323 to L-890; C-324 to L-890; G-325 to L-890; Q-326 to L-890; E-327 to L-890; G-328 to L-890; L-329 to L-890; C-330 to L-890; D-331 to L-890; T-332 to L-890; L-333 to L-890; G-334 to L-890; V-335 to L-890; A-336 to L-890; D-337 to L-890; I-338 to L-890; G-339 to L-890; T-340 to L-890; I-341 to L-890; C-342 to L-890; D-343 to L-890; P-344 to L-890; N-345 to L-890; K-346 to L-890; S-347 to L-890; C-348 to L-890; S-349 to L-890; V-350 to L-890; I-351 to L-890; E-352 to L-890; D-353 to L-890; E-354 to L-890; G-355 to L-890; L-356 to L-890; Q-357 to L-890; A-358 to L-890; A-359 to L-890; H-360 to L-890; T-361 to L-890; L-362 to L-890; A-363 to L-890; H-364 to L-890; E-365 to L-890; L-366 to L-890; G-367 to L-890; H-368 to L-890; V-369 to L-890; L-370 to L-890; S-371 to L-890; M-372 to L-890; P-373 to L-890; H-374 to L-890; D-375 to L-890; D-376 to L-890; S-377 to L-890; K-378 to L-890; P-379 to L-890; C-380 to L-890; T-381 to L-890; R-382 to L-890; L-383 to L-890; F-384 to L-890; G-385 to L-890; P-386 to L-890; M-387 to L-890; G-388 to L-890; K-389 to L-890; H-390 to L-890; H-391 to L-890; V-392 to L-890; M-393 to L-890; A-394 to L-890; P-395 to L-890; L-396 to L-890; F-397 to L-890; V-398 to L-890; H-399 to L-890; L-400 to L-890; N-401 to L-890; Q-402 to L-890; T-403 to L-890; L-404 to L-890; P-405 to L-890; W-406 to L-890; S-407 to L-890; P-408 to L-890; C-409 to L-890; S-410 to L-890; A-411 to L-890; M-412 to L-890; Y-413 to L-890; L-414 to L-890; T-415 to L-890; E-416 to L-890; L-417 to L-890; L-418 to L-890; D-419 to L-890; G-420 to L-890; G-421 to L-890; H-422 to L-890; G-423 to L-890; D-424 to L-890; C-425 to L-890; L-426 to L-890; L-427 to L-890; D-428 to L-890; A-429 to L-890; P-430 to L-890; G-431 to L-890; A-432 to L-890; A-433 to L-890; L-434 to L-890; P-435 to L-890; L-436 to L-890; P-437 to L-890; T-438 to L-890; G-439 to L-890; L-440 to L-890; P-441 to L-890; G-442 to L-890; R-443 to L-890; M-444 to L-890; A-445 to L-890; L-446 to L-890; Y-447 to L-890; Q-448 to L-890; L-449 to L-890; D-450 to L-890; Q-451 to L-890; Q-452 to L-890; C-453 to L-890; R-454 to L-890; Q-455 to L-890; 1456 to L-890; F-457 to L-890; G-458 to L-890; P-459 to L-890; D-460 to L-890; F-461 to L-890; R-462 to L-890; H-463 to L-890; C-464 to L-890; P-465 to L-890; N-466 to L-890; T-467 to L-890; S-468 to L-890; A-469 to L-890; Q-470 to L-890; D-471 to L-890; V-472 to L-890; C-473 to L-890; A-474 to L-890; Q-475 to L-890; L-476 to L-890; W-477 to L-890; 0 C-478 to L-890; H-479 to L-890; T-480 to L-890; D-481 to L-890; G-482 to L-890; A-483 to L-890; E-484 to L-890; P-485 to L-890; L-486 to L-890; C-487 to L-890; H-488 to L-890; T-489 to L-890; K-490 to L-890; N-491 to L-890; G-492 to L-890; S-493 to L-890; L-494 to L-890; P-495 to L-890; W-496 to L-890; A-497 to L-890; D-498 to L-890; G-499 to L-890; T-500 to L-890; P-501 to L-890; C-502 to L-890; G-503 to L-890; P-504 to L-890; G-505 to L-890; H-506 to L-890; L-507 to L-890; C-508 to L-890; S-509 to L-890; E-510 to L-890; G-511 to L-890; S-512 to L-890; C-513 to L-890; L-514 to L-890; P-515 to L-890; E-516 to L-890; E-517 to L-890; E-518 to L-890; V-519 to L-890; E-520 to L-890; R-521 to L-890; P-522 to L-890; K-523 to L-890; P-524 to L-890; V-525 to L-890; V-526 to L-890; D-527 to L-890; G-528 to L-890; G-529 to L-890; W-530 to L-890; A-531 to L-890; P-532 to L-890; W-533 to L-890; G-534 to L-890; P-535 to L-890; W-536 to L-890; G-537 to L-890; E-538 to L-890; C-539 to L-890; S-540 to L-890; R-541 to L-890; T-542 to L-890; C-543 to L-890; G-544 to L-890; G-545 to L-890; G-546 to L-890; V-547 to L-890; Q-548 to L-890; F-549 to L-890; S-550 to L-890; H-551 to L-890; R-552 to L-890; E-553 to L-890; C-554 to L-890; K-555 to L-890; D-556 to L-890; P-557 to L-890; E-558 to L-890; P-559 to L-890; Q-560 to L-890; N-561 to L-890; G-562 to L-890; G-563 to L-890; R-564 to L-890; Y-565 to L-890; C-566 to L-890; L-567 to L-890; G-568 to L-890; R-569 to L-890; R-570 to L-890; A-571 to L-890; K-572 to L-890; Y-573 to L-890; Q-574 to L-890; S-575 to L-890; C-576 to L-890; H-577 to L-890; T-578 to L-890; E-579 to L-890; E-580 to L-890; C-581 to L-890; P-582 to L-890; P-583 to L-890; D-584 to L-890; G-585 to L-890; K-586 to L-890; S-587 to L-890; F-588 to L-890; R-589 to L-890; E-590 to L-890; Q-591 to L-890; Q-592 to L-890; C-593 to L-890; E-594 to L-890; K-595 to L-890; Y-596 to L-890; N-597 to L-890; A-598 to L-890; Y-599 to L-890; N-600 to L-890; Y-601 to L-890; T-602 to L-890; D-603 to L-890; M-604 to L-890; D-605 to L-890; G-606 to L-890; N-607 to L-890; L-608 to L-890; L-609 to L-890; Q-610 to L-890; W-611 to L-890; V-612 to L-890; P-613 to L-890; K-614 to L-890; Y-615 to L-890; A-616 to L-890; G-617 to L-890; V-618 to L-890; S-619 to L-890; P-620 to L-890; R-621 to L-890; D-622 to L-890; R-623 to L-890; C-624 to L-890; K-625 to L-890; L-626 to L-890; F-627 to L-890; C-628 to L-890; R-629 to L-890; A-630 to L-890; R-631 to L-890; G-632 to L-890; R-633 to L-890; S-634 to L-890; E-635 to L-890; F-636 to L-890; K-637 to L-890; V-638 to L-890; F-639 to L-890; E-640 to L-890; A-641 to L-890; K-642 to L-890; V-643 to L-890; I-644 to L-890; D-645 to L-890; G-646 to L-890; T-647 to L-890; L-648 to L-890; C-649 to L-890; G-650 to L-890; P-651 to L-890; E-652 to L-890; T-653 to L-890; L-654 to L-890; A-655 to L-890; I-656 to L-890; C-657 to L-890; V-658 to L-890; R-659 to L-890; G-660 to L-890; Q-661 to L-890; C-662 to L-890; V-663 to L-890; K-664 to L-890; A-665 to L-890; G-666 to L-890; C-667 to L-890; D-668 to L-890; H-669 to L-890; V-670 to L-890; V-671 to L-890; D-672 to L-890; S-673 to L-890; P-674 to L-890; R-675 to L-890; K-676 to L-890; L-677 to L-890; D-678 to L-890; K-679 to L-890; C-680 to L-890; G-681 to L-890; V-682 to L-890; C-683 to L-890; G-684 to L-890; G-685 to L-890; K-686 to L-890; G-687 to L-890; N-688 to L-890; S-689 to L-890; C-690 to L-890; R-691 to L-890; K-692 to L-890; V-693 to L-890; S-694 to L-890; G-695 to L-890; S-696 to L-890; L-697 to L-890; T-698 to L-890; P-699 to L-890; T-700 to L-890; N-701 to L-890; Y-702 to L-890; G-703 to L-890; Y-704 to L-890; N-705 to L-890; D-706 to L-890; I-707 to L-890; V-708 to L-890; T-709 to L-890; I-710 to L-890; P-711 to L-890; A-712 to L-890; G-713 to L-890; A-714 to L-890; T-715 to L-890; N-716 to L-890; I-717 to L-890; D-718 to L-890; V-719 to L-890; K-720 to L-890; Q-721 to L-890; R-722 to L-890; S-723 to L-890; H-724 to L-890; P-725 to L-890; G-726 to L-890; V-727 to L-890; Q-728 to L-890; N-729 to L-890; D-730 to L-890; G-731 to L-890; N-732 to L-890; Y-733 to L-890; L-734 to L-890; A-735 to L-890; L-736 to L-890; K-737 to L-890; T-738 to L-890; A-739 to L-890; D-740 to L-890; G-741 to L-890; Q-742 to L-890; Y-743 to L-890; L-744 to L-890; L-745 to L-890; N-746 to L-890; G-747 to L-890; N-748 to L-890; L-749 to L-890; A-750 to L-890; I-751 to L-890; S-752 to L-890; A-753 to L-890; I-754 to L-890; E-755 to L-890; Q-756 to L-890; D-757 to L-890; I-758 to L-890; L-759 to L-890; V-760 to L-890; K-761 to L-890; G-762 to L-890; T-763 to L-890; I-764 to L-890; L-765 to L-890; K-766 to L-890; Y-767 to L-890; S-768 to L-890; G-769 to L-890; S-770 to L-890; I-771 to L-890; A-772 to L-890; T-773 to L-890; L-774 to L-890; E-775 to L-890; R-776 to L-890; L-777 to L-890; Q-778 to L-890; S-779 to L-890; F-780 to L-890; R-781 to L-890; P-782 to L-890; L-783 to L-890; P-784 to L-890; E-785 to L-890; P-786 to L-890; L-787 to L-890; T-788 to L-890; V-789 to L-890; Q-790 to L-890; L-791 to L-890; L-792 to L-890; T-793 to L-890; V-794 to L-890; P-795 to L-890; G-796 to L-890; E-797 to L-890; V-798 to L-890; F-799 to L-890; P-800 to L-890; P-801 to L-890; K-802 to L-890; V-803 to L-890; K-804 to L-890; Y-805 to L-890; T-806 to L-890; F-807 to L-890; F-808 to L-890; V-809 to L-890; P-810 to L-890; N-811 to L-890; D-812 to L-890; V-813 to L-890; D-814 to L-890; F-815 to L-890; S-816 to L-890; M-817 to L-890; Q-818 to L-890; S-819 to L-890; S-820 to L-890; K-821 to L-890; E-822 to L-890; R-823 to L-890; A-824 to L-890; T-825 to L-890; T-826 to L-890; N-827 to L-890; I-828 to L-890; I-829 to L-890; Q-830 to L-890; P-831 to L-890; L-832 to L-890; L-833 to L-890; H-834 to L-890; A-835 to L-890; Q-836 to L-890; W-837 to L-890; V-838 to L-890; L-839 to L-890; G-840 to L-890; D-841 to L-890; W-842 to L-890; S-843 to L-890; E-844 to L-890; C-845 to L-890; S-846 to L-890; S-847 to L-890; T-848 to L-890; C-849 to L-890; G-850 to L-890; A-851 to L-890; G-852 to L-890; W-853 to L-890; Q-854 to L-890; R-855 to L-890; R-856 to L-890; T-857 to L-890; V-858 to L-890; E-859 to L-890; C-860 to L-890; R-861 to L-890; D-862 to L-890; P-863 to L-890; S-864 to L-890; G-865 to L-890; Q-866 to L-890; A-867 to L-890; S-868 to L-890; A-869 to L-890; T-870 to L-890; C-871 to L-890; N-872 to L-890; K-873 to L-890; A-874 to L-890; L-875 to L-890; K-876 to L-890; P-877 to L-890; E-878 to L-890; D-879 to L-890; A-880 to L-890; K-881 to L-890; P-882 to L-890; C-883 to L-890; E-884 to L-890; S-885 to L-890; of SEQ ID NO:4.

Moreover, C-terminal deletions of the METH2 polypeptide can also be described by the general formula 1−n₂, where n₂ is an integer from 2 to 890 where n corresponds to the position of amino acid residue identified in SEQ ID NO:4. Preferably, C-terminal deletions of the METH2 polypeptide of the invention shown as SEQ ID NO:4 include polypeptides comprising the amino acid sequence of residues: M-1 to P-889; M-1 to C-888; M-1 to L-887; M-1 to Q-886; M-1 to S-885; M-1 to E-884; M-1 to C-883; M-1 to P-882; M-1 to K-881; M-1 to A-880; M-1 to D-879; M-1 to E-878; M-1 to P-877; M-1 to K-876; M-1 to L-875; M-1 to A-874; M-1 to K-873; M-1 to N-872; M-1 to C-871; M-1 to T-870; M-1 to A-869; M-1 to S-868; M-1 to A-867; M-1 to Q-866; M-1 to G-865; M-1 to S-864; M-1 to P-863; M-1 to D-862; M-1 to R-861; M-1 to C-860; M-1 to E-859; M-1 to V-858; M-1 to T-857; M-1 to R-856; M-1 to R-855; M-1 to Q-854; M-1 to W-853; M-1 to G-852; M-1 to A-851; M-1 to G-850; M-1 to C-849; M-1 to T-848; M-1 to S-847; M-1 to S-846; M-1 to C-845; M-1 to E-844; M-1 to S-843; M-1 to W-842; M-1 to D-841; M-1 to G-840; M-1 to L-839; M-1 to V-838; M-1 to W-837; M-1 to Q-836; M-1 to A-835; M-1 to H-834; M-1 to L-833; M-1 to L-832; M-1 to P-831; M-1 to Q-830; M-1 to I-829; M-1 to I-828; M-1 to N-827; M-1 to T-826; M-1 to T-825; M-1 to A-824; M-1 to R-823; M-1 to E-822; M-1 to K-821; M-1 to S-820; M-1 to S-819; M-1 to Q-818; M-1 to M-817; M-1 to S-816; M-1 to F-815; M-1 to D-814; M-1 to V-813; M-1 to D-812; M-1 to N-811; M-1 to P-810; M-1 to V-809; M-1 to F-808; M-1 to F-807; M-1 to T-806; M-1 to Y-805; M-1 to K-804; M-1 to V-803; M-1 to K-802; M-1 to P-801; M-1 to P-800; M-1 to F-799; M-1 to V-798; M-1 to E-797; M-1 to G-796; M-1 to P-795; M-1 to V-794; M-1 to T-793; M-1 to L-792; M-1 to L-791; M-1 to Q-790; M-1 to V-789; M-1 to T-788; M-1 to L-787; M-1 to P-786; M-1 to E-785; M-1 to P-784; M-1 to L-783; M-1 to P-782; M-1 to R-781; M-1 to F-780; M-1 to S-779; M-1 to Q-778; M-1 to L-777; M-1 to R-776; M-1 to E-775; M-1 to L-774; M-1 to T-773; M-1 to A-772; M-1 to I-771; M-1 to S-770; M-1 to G-769; M-1 to S-768; M-1 to Y-767; M-1 to K-766; M-1 to L-765; M-1 to I-764; M-1 to T-763; M-1 to G-762; M-1 to K-761; M-1 to V-760; M-1 to L-759; M-1 to I-758; M-1 to D-757; M-1 to Q-756; M-1 to E-755; M-1 to I-754; M-1 to A-753; M-1 to S-752; M-1 to I-751; M-1 to A-750; M-1 to L-749; M-1 to N-748; M-1 to G-747; M-1 to N-746; M-1 to L-745; M-1 to L-744; M-1 to Y-743; M-1 to Q-742; M-1 to G-741; M-1 to D-740; M-1 to A-739; M-1 to T-738; M-1 to K-737; M-1 to L-736; M-1 to A-735; M-1 to L-734; M-1 to Y-733; M-1 to N-732; M-1 to G-731; M-1 to D-730; M-1 to N-729; M-1 to Q-728; M-1 to V-727; M-1 to −726; M-1 to P-725; M-1 to H-724; M-1 to S-723; M-1 to R-722; M-1 to Q-721; M-1 to K-720; M-1 to V-719; M-1 to D-718; M-1 to I-717; M-1 to N-716; M-1 to T-715; M-1 to A-714; M-1 to G-713; M-1 to A-712; M-1 to P-711; M-1 to I-710; M-1 to T-709; M-1 to V-708; M-1 to I-707; M-1 to D-706; M-1 to N-705; M-1 to Y-704; M-1 to G-703; M-1 to Y-702; M-1 to N-701; M-1 to T-700; M-1 to P-699; M-1 to T-698; M-1 to L-697; M-1 to S-696; M-1 to G-695; M-1 to S-694; M-1 to V-693; M-1 to K-692; M-1 to R-691; M-1 to C-690; M-1 to S-689; M-1 to N-688; M-1 to G-687; M-1 to K-686; M-1 to G-685; M-1 to G-684; M-1 to C-683; M-1 to V-682; M-1 to G-681; M-1 to C-680; M-1 to K-679; M-1 to D-678; M-1 to L-677; M-1 to K-676; M-1 to R-675; M-1 to P-674; M-1 to S-673; M-1 to D-672; M-1 to V-671; M-1 to V-670; M-1 to H-669; M-1 to D-668; M-1 to C-667; M-1 to G-666; M-1 to A-665; M-1 to K-664; M-1 to V-663; M-1 to C-662; M-1 to Q-661; M-1 to G-660; M-1 to R-659; M-1 to V-658; M-1 to C-657; M-1 to I-656; M-1 to A-655; M-1 to L-654; M-1 to T-653; M-1 to E-652; M-1 to P-651; M-1 to G-650; M-1 to C-649; M-1 to L-648; M-1 to T-647; M-1 to G-646; M-1 to D-645; M-1 to I-644; M-1 to V-643; M-1 to K-642; M-1 to A-641; M-1 to E-640; M-1 to F-639; M-1 to V-638; M-1 to K-637; M-1 to F-636; M-1 to E-635; M-1 to S-634; M-1 to R-633; M-1 to G-632; M-1 to R-631; M-1 to A-630; M-1 to R-629; M-1 to C-628; M-1 to F-627; M-1 to L-626; M-1 to K-625; M-1 to C-624; M-1 to R-623; M-1 to D-622; M-1 to R-621; M-1 to P-620; M-1 to S-619; M-1 to V-618; M-1 to G-617; M-1 to A-616; M-1 to Y-615; M-1 to K-614; M-1 to P-613; M-1 to V-612; M-1 to W-611; M-1 to Q-610; M-1 to L-609; M-1 to L-608; M-1 to N-607; M-1 to G-606; M-1 to D-605; M-1 to M-604; M-1 to D-603; M-1 to T-602; M-1 to Y-601; M-1 to N-600; M-1 to Y-599; M-1 to A-598; M-1 to N-597; M-1 to Y-596; M-1 to K-595; M-1 to E-594; M-1 to C-593; M-1 to Q-592; M-1 to Q-591; M-1 to E-590; M-1 to R-589; M-1 to F-588; M-1 to S-587; M-1 to K-586; M-1 to G-585; M-1 to D-584; M-1 to P-583; M-1 to P-582; M-1 to C-581; M-1 to E-580; M-1 to E-579; M-1 to T-578; M-1 to H-577; M-1 to C-576; M-1 to S-575; M-1 to Q-574; M-1 to Y-573; M-1 to K-572; M-1 to A-571; M-1 to R-570; M-1 to R-569; M-1 to G-568; M-1 to L-567; M-1 to C-566; M-1 to Y-565; M-1 to R-564; M-1 to G-563; M-1 to G-562; M-1 to N-561; M-1 to Q-560; M-1 to P-559; M-1 to E-558; M-1 to P-557; M-1 to D-556; M-1 to K-555; M-1 to C-554; M-1 to E-553; M-1 to R-552; M-1 to H-551; M-1 to S-550; M-1 to F-549; M-1 to Q-548; M-1 to V-547; M-1 to G-546; M-1 to G-545; M-1 to G-544; M-1 to C-543; M-1 to T-542; M-1 to R-541; M-1 to S-540; M-1 to C-539; M-1 to E-538; M-1 to G-537; M-1 to W-536; M-1 to P-535; M-1 to G-534; M-1 to W-533; M-1 to P-532; M-1 to A-531; M-1 to W-530; M-1 to G-529; M-1 to G-528; M-1 to D-527; M-1 to V-526; M-1 to V-525; M-1 to P-524; M-1 to K-523; M-1 to P-522; M-1 to R-521; M-1 to E-520; M-1 to V-519; M-1 to E-518; M-1 to E-517; M-1 to β-516; M-1 to P-515; M-1 to L-514; M-1 to C-513; M-1 to S -512; M-1 to G-511; M-1 to E-510; M-1 to S-509; M-1 to C-508; M-1 to L-507; M-1 to S-506; M-1 to G-505; M-1 to P-504; M-1 to G-503; M-1 to C-502; M-1 to P-501; M-1 to T-500; M-1 to G-499; M-1 to D-498; M-1 to A-497; M-1 to W-496; M-1 to P-495; M-1 to L-494; M-1 to S-493; M-1 to G-492; M-1 to N-491; M-1 to K-490; M-1 to T-489; M-1 to H-488; M-1 to C-487; M-1 to L-486; M-1 to P-485; M-1 to E-484; M-1 to A-483; M-1 to G-482; M-1 to D-481; M-1 to T-480; M-1 to H-479; M-1 to C-478; M-1 to W-477; M-1 to L-476; M-1 to Q-475; M-1 to A-474; M-1 to C-473; M-1 to V-472; M-1 to D-471; M-1 to Q-470; M-1 to A-469; M-1 to S-468; M-1 to T-467; M-1 to N-466; M-1 to P-465; M-1 to C-464; M-1 to H-463; M-1 to R-462; M-1 to F-461; M-1 to D-460; M-1 to P-459; M-1 to G-458; M-1 to F-457; M-1 to I-456; M-1 to Q-455; M-1 to R-454; M-1 to C-453; M-1 to Q-452; M-1 to Q-451; M-1 to D-450; M-1 to L-449; M-1 to Q-448; M-1 to Y-447; M-1 to L-446; M-1 to A-445; M-1 to M-444; M-1 to R-443; M-1 to G-442; M-1 to P-441; M-1 to L-440; M-1 to G-439; M-1 to T-438; M-1 to P-437; M-1 to L-436; M-1 to P-435; M-1 to L-434; M-1 to A-433; M-1 to A-432; M-1 to G-431; M-1 to P-430; M-1 to A-429; M-1 to D-428; M-1 to L-427; M-1 to L-426; M-1 to C-425; M-1 to D-424; M-1 to G-423; M-1 to H-1422; M-1 to G-421; M-1 to G-420; M-1 to D-419; M-1 to L-418; M-1 to L-417; M-1 to E-416; M-1 to T-415; M-1 to L-414; M-1 to Y-413; M-1 to M-412; M-1 to A-411; M-1 to S-410; M-1 to C-409; M-1 to P-408; M-1 to S-407; M-1 to W-406; M-1 to P-405; M-1 to L-404; M-1 to T-403; M-1 to Q-402; M-1 to N-401; M-1 to L-400; M-1 , to H-399; M-1 to V-398; M-1 to F-397; M-1 to L-396; M-1 to P-395; M-1 to A-394; M-1 to M-393; M-1 to V-392; M-1 to H-391; M-1 to H-390; M-1 to K-389; M-1 to G-388; M-1 to M-387; M-1 to P-386; M-1 to G-385; M-1 to F-384; M-1 to L-383; M-1 to R-382; >M-1 to T-381; M-1 to C-380; M-1 to P-379; M-1 to K-378; M-1 to S-377; M-1 to D-376; M-1 to D-375; M-1 to H-374; M-1 to P-373; M-1 to M-372; M-1 to S-371; M-1 to L-370; M-1 to V-369; M-1 to H-368; M-1 to G-367; M-1 to L-366; M-1 to E-365; M-1 to H-364; M-1 to A-363; M-1 to L-362; M-1 to T-361; M-1 to H-360; M-1 to A-359; M-1 to A-358; M-1 to Q-357; M-1 to L-356; M-1 to G-355; M-1 to E-354; M-1 to D-353; M-1 to E-352; M-1 to I-351; M-1 to V-350; M-1 to S-349; M-1 to C-348; M-1 to S-347; M-1 to K-346; M-1 to N-345; M-1 to P-344; M-1 to D-343; M-1 to C-342; M-1 to I-341; M-1 to T-340; M-1 to G-339; M-1 to I-338; M-1 to D-337; M-1 to A-336; M-1 to V-335; M-1 to G-334; M-1 to L-333; M-1 to T-332; M-1 to D-331; M-1 to C-330; M-1 to L-329; M-1 to G-328; M-1 to E-327; M-1 to Q-326; M-1 to G-325; M-1 to C-324; M-1 to F-323; M-1 to N-322; M-1 to Q-321; M-1 to R-320; M-1 to T-319; M-1 to L-318; M-1 to L-317; M-1 to I-316; M-1 to A-315; M-1 to T-314; M-1 to D-313; M-1 to Y-312; M-1 to H-311; M-1 to E-310; M-1 to P-309; M-1 to H-308; M-1 to R-307; M-1 to D-306; M-1 to S-305; M-1 to P-304; M-1 to Q-303; M-1 to N-302; M-1 to F-301; M-1 to R-300; M-1 to R-299; M-1 to Q-298; M-1 to W-297; M-1 to N-296; M-1 to C-295; M-1 to F-294; M-1 to N-293; M-1 to R-292; M-1 to L-291; M-1 to T-290; M-1 to L-289; M-1 to G-288; M-1 to G-287; M-1 to N-286; M-1 to D-285; M-1 to S-284; M-1 to V-283; M-1 to E-282; M-1 to P-281; M-1 to G-280; M-1 to W-279; M-1 to K-278; M-1 to E-277; M-1 to D-276; M-1 to E-275; M-1 to V-274; M-1 to I-273; M-1 to L-272; M-1 to V-271; M-1 to K-270; M-1 to V-269; M-1 to V-268; M-1 to M-267; M-1 to L-266; M-1 to N-265; M-1 to I-264; M-1 to S-263; M-1 to N-262; M-1 to K-261; M-1 to I-260; M-1 to S-259; M-1 to P-258; M-1 to H-257; M-1 to K-256; M-1 to Y-255; M-1 to I-254; M-1 to R-253; M-1 to A-252; M-1 to A-251; M-1 to V-250; M-1 to S-249; M-1 to M-248; M-1 to L-247; M-1 to T-246; M-1 to L-245; M-1 to I-244; M-1 to H-243; M-1 to N-242; M-1 to Q-241; M-1 to L-240; M-1 to D-239; M-1 to A-238; M-1 to G-237; M-1 to Y-236; M-1 to F-235; M-1 : to A-234; M-1 to A-233; M-1 to M-232; M-1 to S-231; M-1 to A-230; M-1 to D-229; M-1 to A-228; M-1 to V-227; M-1 to L-226; M-1 to L-225; M-1 to T-224; M-1 to E-223; M-1 to V-222; M-1 to F-221; M-1 to R-220; M-1 to A-219; M-1 to E-218; M-1 to S-217; M-1 to V-216; M-1 to F-215; M-1 to R-214; M-1 to K-213; M-1 to T-212; M-1 to R-211; M-1 to S-210; M-1 to T-209; M-1 to A-208; M-1 to G-207; M-1 to L-206; M-1 to P-205; M-1 to P-204; M-1 to P-203; M-1 to P-202; M-1 to E-201; M-1 to S-200; M-1 to A-199; M-1 to G-198; M-1 to E-197; M-1 to A-196; M-1 to E-195; M-1 to E-194; M-1 to E-193; M-1 to Q-192; M-1 to S-191; M-1 to E-190; M-1 to E-189; M-1 to E-188; M-1 to S-187; M-1 to D-186; M-1 to E-185; M-1 to Q-184; M-1 to H-183; M-1 to D-182; M-1 to G-181; M-1 to R-180; M-1 to E-179; M-1 to Q-178; M-1 to R-177; M-1 to Q-176; M-1 to G-175; M-1 to E-174; M-1 to G-173; M-1 to T-172; M-1 to E-171; M-1 to V-170; M-1 to E-169; M-1 to W-168; M-1 to E-167; M-1 to P-166; M-1 to G-165; M-1 to R-164; M-1 to P-163; M-1 to L-162; M-1 to P-161; M-1 to R-160; M-1 to A-159; M-1 to G-158; M-1 to A-157; M-1 to P-156; M-1 to G-155; M-1 to W-154; M-1 to R-153; M-1 to Q-152; M-1 to L-151; M-1 to R-150; M-1 to H-149; M-1 to P-148; M-1 to Q-147; M-1 to A-146; M-1 to L-145; M-1 to S-144; M-1 to G-143; M-1 to G-142; M-1 to A-141; M-1 to G-140; M-1 to Q-139; M-1 to P-138; M-1 to Q-137; M-1 to I-136; M-1 to T-135; M-1 to F-134; M-1 to E-133; M-1 to E-132; M-1 to G-131; M-1 to D-130; M-1 to L-129; M-1 to L-128; M-1 to F-127; M-1 to S-126; M-1 to G-125; M-1 to S-124; M-1 to L-123; M-1 to G-122; M-1 to R-121; M-1 to C-120; M-1 to L-119; M-1 to S-118; M-1 to V-117; M-1 to A-116; M-1 to A-115; M-1 to L-114; M-1 to S-113; M-1 to E-112; M-1 to P-111; M-1 to E-110; M-1 to G-109; M-1 to N-108; M-1 to V-107; M-1 to T-106; M-1 to G-105; M-1 to S-104; M-1 to F-103; M-1 to F-102; M-1 to C-101; M-1 to G-100; M-1 to R-99; M-1 to L-98; M-1 to G-97; M-1 to R-96; M-1 to E-95; M-1 to G-94; M-1 to G-93; M-1 to T-92; M-1 to A-91; M-1 to R-90; M-1 to G-89; M-1 to S-88; M-1 to G-87; M-1 to G-86; M-1 to L-85; M-1 to R-84; M-1 to E-83; M-1 to I-82; M-1 to K-81; M-1 to F-80; M-1 to E-79; M-1 to P-78; M-1 to A-77; M-1 to L-76; M-1 to F-75; M-1 to S-74; M-1 to D-73; M-1 to D-72; M-1 to P-71; M-1 to A-70; M-1 to L-69; M-1 to R-68; M-1 to L-67; M-1 to V-66; M-1 to F-65; M-1 to G-64; M-1 to K-63; M-1 to G-62; M-1 to F-61; M-1 to A-60; M-1 to S-59; M-1 to L-58; M-1 to H-57; M-1 to L-56; M-1 to A-55; M-1 to L-54; M-1 to E-53; M-1 to G-52; M-1 to A-51; M-1 to S-50; M-1 to G-49; M-1 to P-48; M-1 to L-47; M-1 to R-46; M-1 to T-45; M-1 to P-44; M-1 to V-43; M-1 to V-42; M-1 to L-41; M-1 to E-40; M-1 to S-39; M-1 to A-38; M-1 to Q-37; M-1 to G-36; M-1 to G-35; M-1 to A-34; M-1 to A-33; M-1 to P-32; M-1 to R-31; M-1 to A-30; M-1 to P-29; M-1 to A-28; M-1 to G-27; M-1 to R-26; M-1 to A-25; M-1 to L-24; M-1 to P-23; M-1 to L-22; M-1 to L-21; M-1 to L-20; M-1 to L-19; M-1 to L-18; M-1 to L-17; M-1 to L-16; M-1 to L-15; M-1 to L-14; M-1 to F-13; M-1 to P-12; M-1 to L-11; M-1 to W-10; M-1 to R-9; M-1 to P-8; M-1 to A-7; of SEQ ID NO:4. Preferably, any of the above listed N- or C-terminal deletions can be combined to produce a N- and C-terminal deleted METH2 polypeptide.

The invention also provides polypeptides having one or more amino acids deleted from both the amino and the carboxyl termini, which may be described generally as having residues m₁-n₁ of SEQ ID NO:2 or m₂-n₂ SEQ ID NO:4, where n and m are integers as described above.

The invention also provides mutants of the metalloprotease domain of METH1, which are described by the general formula m₃-n₃, where m₃ is an integer from 205 to 265, and n₃ is an integer from 285 to 950, where m₃ and n₃ correspond to the position of the amino acid residue identified in SEQ ID NO:2. The invention further provides mutants of the metalloprotease domain of METH1, which are described by the general formula m₄-n₄, where m₄ is an integer from 1 to 409, and n₄ is an integer from 429 to 489, where m₄ and n₄ correspond to the position of the amino acid residue identified in SEQ ID NO:2.

The invention also provides mutants of the disintegrin domain of METH1, which are described by the general formula m₅-n₅, where m₅ is an integer from 430 to 490, and n₅ is an integer from 510 to 950, where m₅ and n₅ correspond to the position of the amino acid residue identified in SEQ ID NO:2.

The invention further provides mutants of the disintegrin domain of METH1, which are described by the general formula m₆-n₆, where m₆ is an integer from 1 to 494, and n₆ is an integer from 514 to 574, where m₆ and n₆ correspond to the position of the amino acid residue identified in SEQ ID NO:2.

The invention further provides mutants of the TSP1 domain of METH1, which are described by the general formula m₇-n₇, where m₇ is an integer from 515 to 575, and n₇ is an integer from 595 to 950, where m₇ an n₇ correspond to the position of the amino acid residue identified in SEQ ID NO:2.

The invention also provides mutants of the TSP1 domain of METH1, which are described by the general formula m₈-n₈, where m₈ is an integer from 1 to 548, and n₈ is an integer from 568 to 628, where m₈ and n₈ correspond to the position of the amino acid residue identified in SEQ ID NO:2.

The invention further provides mutants of the TSP2 domain of METH1, which are described by the general formula m₉-n₉, where m₉ is an integer from 801 to 871, and n₉ is an integer from 891 to 950, where m₉ and n₉ correspond to the position of the amino acid residue identified in SEQ ID NO:2.

The invention also provides mutants of the TSP2 domain of METH1, which are described by the general formula m₁₀-n₁₀, where m₁₀ is an integer from 1 to 834, and n₁o is an integer from 864 to 924, where m₁₀ and n₁₀ correspond to the position of the amino acid residue identified in SEQ ID NO:2.

The invention further provides mutants of the TSP3 domain of METH1, which are described by the general formula m₁₁-n₁₁, where m₁₁ is an integer from 865 to 925, and n₁₁ is an integer from 945 to 950, where m₁₁ and n₁₁ correspond to the position of the amino acid residue identified in SEQ ID NO:2. The invention also provides mutants of the TSP3 domain of METH1, which are described by the general formula m₁₂-n₁₂, where m₁₂ is an integer from 1 to 884, and n₁₂ is an integer from 904 to 950, where m₁₂ and n₁₂ correspond to the position of the amino acid residue identified in SEQ ID NO:2.

The invention further provides mutants of the metalloprotease domain of METH2, which are described by the general formula m₁₃-n₁₃, where m₁₃ is an integer from 184 to 244, and n₁₃ is an integer from 264 to 890, where m₁₃ and n₁₃ correspond to the position of the amino acid residue identified in SEQ ID NO:4. The invention also provides mutants of the metalloprotease domain of METH2, which are described by the general formula m₁₄-n₁₄, where m₁₄ is an integer from 1 to 389, and n₁₄ is an integer from 409 to 469, where m₁₄ and n₁₄ correspond to the position of the amino acid residue identified in SEQ ID NO:4.

The invention further provides mutants of the disintegrin domain of METH2, which are described by the general formula m₁₅-n₁₅, where m₁₅ is an integer from 400 to 470, and n₁₅ is an integer from 490 to 890, where m₁₅ and n₁₅ correspond to the position of the amino acid residue identified in SEQ ID NO:4. The invention also provides mutants of the disintegrin domain of METH2, which are described by the general formula 11 m₁₆-n₁₆, where m₁₆ is an integer from 1 to 479, and n₁₆ is an integer from 499 to 559, where m₁₆ and n₁₆ correspond to the position of the amino acid residue identified in SEQ ID NO:4.

The invention further provides mutants of the TSP1 domain of METH2, which are described by the general formula m₁₇-n₁₇, where m₁₇ is an integer from 500 to 560, and n₁₇ is an integer from 580 to 890, where m₁₇ and n₁₇ correspond to the position of the amino acid residue identified in SEQ ID NO:4. The invention also provides mutants of the TSP1 domain of METH2, which are described by the general formula m₁₈-n₁₈, where m₁₈ is an integer from 1 to 533, and n₁₈ is an integer from 553 to 613, where m₁₈ and n₁₈ correspond to the position of the amino acid residue identified in SEQ ID NO:4.

The invention further provides mutants of the TSP2 domain of METH2, which are described by the general formula m₁₉-n₁₉, where m₁₉ is an integer from 807 to 867, and n₁₉ is an integer from 887 to 890, where m₁₉ and n₁₉ correspond to the position of the amino acid residue identified in SEQ ID NO:4. The invention also provides mutants of the TSP2 domain of METH2, which are described by the general formula m₂₀-n₂₀, where m₂₀ is an integer from 1 to 840, and n₂₀ is an integer from 860 to 890, where m₂₀ and n₂₀ correspond to the position of the amino acid residue identified in SEQ ID NO:4.

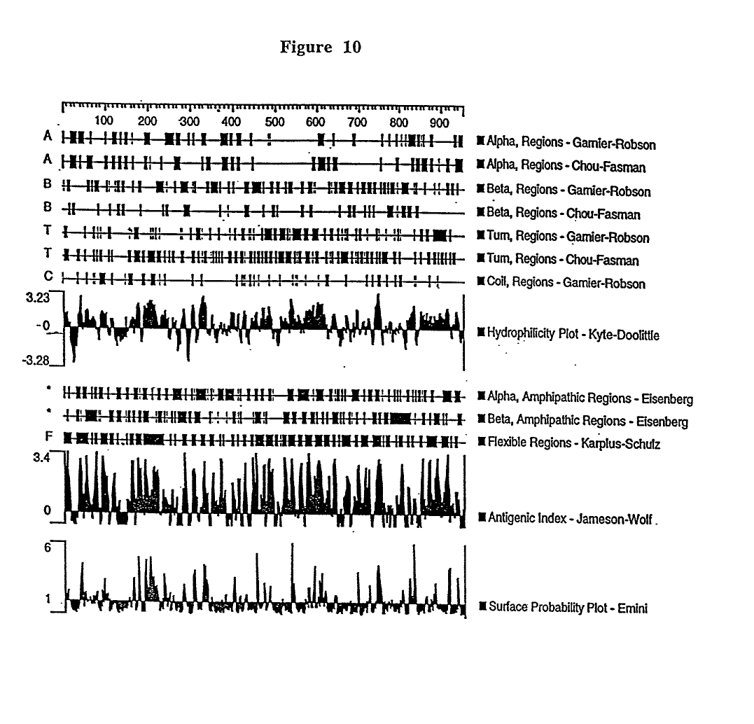

Also preferred are METH1 or METH2 polypeptide and polynucleotide fragments characterized by structural or functional domains. Preferred embodiments of the invention include fragments that comprise alpha-helix and alpha-helix forming regions (“alpha-regions”), beta-sheet and beta-sheet-forming regions (“beta-regions”), turn and turn-forming regions (“turn-regions”), coil and coil-forming regions (“coil-regions”), hydrophilic regions, hydrophobic regions, alpha amphipathic regions, beta amphipathic regions, flexible regions, surface-forming regions, substrate binding region, and high antigenic index regions. As set out in the Figures, such preferred regions include Garnier-Robson alpha-regions, beta-regions, turn-regions, and coil-regions, Chou-Fasman alpha-regions, beta-regions, and turn-regions, Kyte-Doolittle hydrophilic regions and hydrophobic regions, Eisenberg alpha and beta amphipathic regions, Karplus-Schulz flexible regions, Emini surface-forming regions, and Jameson-Wolf high antigenic index regions. Polypeptide fragments of SEQ ID NO:2 falling within conserved domains are specifically contemplated by the present invention. (See FIGS. 10 & 11 and Tables 1& 2.) Moreover, polynucleotide fragments encoding these domains are also contemplated.

Other preferred fragments are biologically active METH1 or METH2 fragments. Biologically active fragments are those exhibiting activity similar, but not necessarily identical, to an activity of the METH1 or METH2 polypeptide. The biological activity of the fragments may include an improved desired activity, or a decreased undesirable activity. However, many polynucleotide sequences, such as EST sequences, are publicly available and accessible through sequence databases. Some of these sequences are related to SEQ ID NO: 1 or SEQ ID NO:3 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides comprising a nucleotide sequence described by the general formula of a−b, where a is any integer between 1 to 936 of SEQ ID NO: 1, b is an integer of 15 to 950, where both a and b correspond to the positions of nucleotide residues shown in SEQ ID NO:1, and where the b is greater than or equal to a +14. Moreover, preferably excluded from the present invention are one or more polynucleotides comprising a nucleotide sequence described by the general formula of a−b, where a is any integer between 1 to 876 of SEQ ID NO:3, b is an integer of 15 to 890, where both a and b correspond to the positions of nucleotide residues shown in SEQ ID NO:3, and where the b is greater than or equal to a +14.

The above-described fragments may be used to make fusion proteins, for example Fc or Flag fusion proteins, as described below.

Epitopes & Antibodies

In another aspect, the invention provides peptides and polypeptides comprising epitope-bearing portions of the polypeptides of the present invention. These epitopes are immunogenic or antigenic epitopes of the polypeptides of the present invention. An “immunogenic epitope” is defined as a part of a protein that elicits an antibody response in vivo when the whole polypeptide of the present invention, or fragment thereof, is the immunogen. On the other hand, a region of a polypeptide to which an antibody can bind is defined as an “antigenic determinant” or “antigenic epitope.” The number of in vivo immunogenic epitopes of a protein generally is less than the number of antigenic epitopes. See, e.g., Geysen, et al. (1983) Proc. Natl. Acad. Sci. USA 81:3998-4002. However, antibodies can be made to any antigenic epitope, regardless of whether it is an immunogenic epitope, by using methods such as phage display. See e.g., Petersen G. et al. (1995) Mol. Gen. Genet. 249:425-431. Therefore, included in the present invention are both immunogenic epitopes and antigenic epitopes.

A list of exemplified amino acid sequences comprising immunogenic epitopes are shown in Tables 1 and 2. It is pointed out that Tables 1 and 2 only list amino acid residues comprising epitopes predicted to have the highest degree of antigenicity using the algorithm of Jameson and Wolf, (1988) Comp. Appl. Biosci. 4:181-186 (said references incorporated by reference in their entireties). The Jameson-Wolf antigenic analysis was performed using the computer program PROTEAN, using default parameters (Version 3.11 for the Power Macintosh, DNASTAR, Inc., 1228 South Park Street Madison, Wis.). Tables 1 and 2 and portions of polypeptides not listed in Tables 1 and 2 are not considered non-immunogenic. The immunogenic epitopes of Tables 1 and 2 are exemplified lists, not exhaustive lists, because other immunogenic epitopes are merely not recognized as such by the particular algorithm used. Amino acid residues comprising other immunogenic epitopes may be routinely determined using algorithms similar to the Jameson-Wolf analysis or by in vivo testing for an antigenic response using methods known in the art. See, e.g., Geysen et al., supra; U.S. Pat. Nos. 4,708,781; 5,194,392; 4,433,092; and 5,480,971 (said references incorporated by reference in their entireties).

Antigenic epitope-bearing peptides and polypeptides of the invention preferably contain a sequence of at least seven, more preferably at least nine and most preferably between about 15 to about 30 amino acids contained within the amino acid sequence of a polypeptide of the invention.

Using DNAstar analysis, SEQ ID NO:2 was found antigenic at amino acids: 2-14, 3244, 47-60, 66-78, 87-103, 109-118, 146-162, 168-180, 183-219, 223-243, 275-284, 296-306, 314-334, 341-354, 357-376, 392-399, 401410, 418-429, 438-454, 456-471, 474-488, 510-522, 524-538, 550-561, 565-626, 630-643, 659-671, 679-721, 734-749,784-804, 813-820, 825-832, 845-854, 860-894, 899-917, 919-924 and 928-939.

Using DNAstar analysis, SEQ ID NO:4 was found antigenic at amino acids: 26-38, 45-52,69-76, 80-99, 105-113, 129-136, 138-217, 254-263, 273-289, 294-313, 321-331, 339-356, 371-383, 417-427, 438443, 459-471, 479-505, 507-526, 535-546, 550-607, 615-640, 648-653, 660-667, 669-681, 683-704, 717-732, 737-743, 775-787, 797-804, 811-825, 840-867 and 870-884.

Thus, these regions of METH1 and/or METH2 are non-limiting examples of antigenic polypeptides or peptides that can be used to raise METH1 and/or METH2-specific antibodies include.

It is particularly pointed out that the amino acid sequences of Tables 1 and 2 comprise immunogenic epitopes. Tables 1 and 2 list only the critical residues of immunogenic epitopes determined by the Jameson-Wolf analysis. Thus, additional flanking residues on either the N-terminal, C-terminal, or both N- and C-terminal ends may be added to the sequences of Tables 1 and 2 to generate an epitope-bearing polypeptide of the present invention. Therefore, the immunogenic epitopes of Tables 1 and 2 may include additional N-terminal or C-terminal amino acid residues. The additional flanking amino acid residues may be contiguous flanking N-terminal and/or C-terminal sequences from the polypeptides of the present invention, heterologous polypeptide sequences, or may include both contiguous flanking sequences from the polypeptides of the present invention and heterologous polypeptide sequences.

Polypeptides of the present invention comprising immunogenic or antigenic epitopes are at least 7 amino acids residues in length. “At least” means that a polypeptide of the present invention comprising an immunogenic or antigenic epitope may be 7 amino acid residues in length or any integer between 7 amino acids and the number of amino acid residues of the full length polypeptides of the invention. Preferred polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length. However, it is pointed out that each and every integer between 7 and the number of amino acid residues of the full length polypeptide are included in the present invention.

The immuno and antigenic epitope-bearing fragments may be specified by either the number of contiguous amino acid residues, as described above, or further specified by N-terminal and C-terminal positions of these fragments on the amino acid sequence of SEQ ID NO:2 or 4. Every combination of a N-terminal and C-terminal position that a fragment of, for example, at least 7 or at least 15 contiguous amino acid residues in length could occupy on the amino acid sequence of SEQ ID NO:2 or 4 is included in the invention. Again, “at least 7 contiguous amino acid residues in length” means 7 amino acid residues in length or any integer between 7 amino acids and the number of amino acid residues of the full length polypeptide of the present invention. Specifically, each and every integer between 7 and the number of amino acid residues of the full length polypeptide are included in the present invention.

Immunogenic and antigenic epitope-bearing polypeptides of the invention are useful, for example, to make antibodies which specifically bind the polypeptides of the invention, and in immunoassays to detect the polypeptides of the present invention. The antibodies are useful, for example, in affinity purification of the polypeptides of the present invention. The antibodies may also routinely be used in a variety of qualitative or quantitative immunoassays, specifically for the polypeptides of the present invention using methods known in the art. See, e.g., Harlow et al., Antibodies: A Laboratory Manual, (Cold Spring Harbor Laboratory Press; 2nd Ed. 1988).

The epitope-bearing polypeptides of the present invention may be produced by any conventional means for making polypeptides including synthetic and recombinant methods known in the art. For instance, epitope-bearing peptides may be synthesized using known methods of chemical synthesis. For instance, Houghten has described a simple method for the synthesis of large numbers of peptides, such as 10-20 mgs of 248 individual and distinct 13 residue peptides representing single amino acid variants of a segment of the HA1 polypeptide, all of which were prepared and characterized (by ELISA-type binding studies) in less than four weeks (Houghten, R. A. Proc. Natl. Acad. Sci. USA 82:5131-5135 (1985)). This “Simultaneous Multiple Peptide Synthesis (SMPS)” process is further described in U.S. Pat. No. 4,631,211 to Houghten and coworkers (1986). In this procedure the individual resins for the solid-phase synthesis of various peptides are contained in separate solvent-permeable packets, enabling the optimal use of the many identical repetitive steps involved in solid-phase methods. A completely manual procedure allows 500-1000 or more syntheses to be conducted simultaneously (Houghten et al. (1985) Proc. Natl. Acad. Sci. 82:5131-5135 at 5134).

The present invention encompasses polypeptides comprising, or alternatively consisting of, an epitope of the polypeptide having an amino acid sequence of SEQ ID NO:2 and/or 4, or an epitope of the polypeptide sequence encoded by a polynucleotide sequence contained in ATCC Deposit No. 209581 or 209582 or PTA 1478 or encoded by a polynucleotide that hybridizes to the complement of the sequence of SEQ ID NO: 1 and/or 3 or contained in ATCC Deposit No: 209581 or 209582 or PTA 1478 under stringent hybridization conditions or lower stringency hybridization conditions as defined supra. The present invention further encompasses polynucleotide sequences encoding an epitope of a polypeptide sequence of the invention (such as, for example, the sequence disclosed in SEQ ID NO: 1 or 3) polynucleotide sequences of the complementary strand of a polynucleotide sequence encoding an epitope of the invention, and polynucleotide sequences which hybridize to the complementary strand under stringent hybridization conditions or lower stringency hybridization conditions defined supra.

The term “epitopes,” as used herein, refers to portions of a polypeptide having antigenic or immunogenic activity in an animal, preferably a mammal, and most preferably in a human. In a preferred embodiment, the present invention encompasses a polypeptide comprising an epitope, as well as the polynucleotide encoding this polypeptide. An “immunogenic epitope,” as used herein, is defined as a portion of a protein that elicits an antibody response in an animal, as determined by any method known in the art, for example, by the methods for generating antibodies described infra. (See, for example, Geysen et al., Proc. Natl. Acad. Sci. USA 81:3998-4002 (1983)). The term “antigenic epitope,” as used herein, is defined as a portion of a protein to which an antibody can immunospecifically bind its antigen as determined by any method well known in the art, for example, by the immunoassays described herein. Immunospecific binding excludes non-specific binding but does not necessarily exclude cross-reactivity with other antigens. Antigenic epitopes need not necessarily be immunogenic.

Fragments which function as epitopes may be produced by any conventional means. (See, e.g., Houghten, Proc. Natl. Acad. Sci. USA 82:5131-5135 (1985), further described in U.S. Pat. No. 4,631,211).

In the present invention, antigenic epitopes preferably contain a sequence of at least 4, at least 5, at least 6, at least 7, more preferably at least 8, at least 9, at least 10, at least 11, at least 12, at least 13, at least 14, at least 15, at least 20, at least 25, at least 30, at least 40, at least 50, and, most preferably, between about 15 to about 30 amino acids. Preferred polypeptides comprising immunogenic or antigenic epitopes are at least 10, 15, 20, 25, 30, 35, 40, 45, 50, 55, 60, 65, 70, 75, 80, 85, 90, 95, or 100 amino acid residues in length. Additionally preferred antigenic epitopes of METH1 comprise, or alternatively consist of, the amino acid sequence of residues: M-1 to P-15; G-2 to V-16; N-3 to P-17; A-4 to T-18; E-5 to L-19; R-6 to L-20; A-7 to L-21; P-8 to L-22; G-9 to A-23; S-10 to A-24; R-11 to A-25; S-12 to L-26; F-13 to L-27; G-14 to A-28; P-15 to V-29; V-16 to S-30; P-17 to D-31; T-18 to A-32; L-19 to L-33; L-20 to G-34; L-21 to R-35; L-22 to P -36; A-23 to S-37; A-24 to E-38; A-25 to E-39; L-26 to D-40; L-27 to E-41; A-28 to E-42; V-29 to L-43; S-30 to V-44; D-31 to V-45; A-32 to P-46; L-33 to E 47; G-34 to L-48; R-35 to E-49; P-36 to R-50; S-37 to A-51; E-38 to P-52; E-39 to G-53; D-40 to H-54; E-41 to G-55; E-42 to T-56; L-43 to T-57; V-44 to R -58; V-45 to L-59; P-46 to R-60; E-47 to L-61; L-48 to H-62; E-49 to A-63; R-50 to F-64; A-51 to D-65; P-52 to Q-66; G-53 to Q-67; H-54 to L-68; G-55 to D -69; T-56 to L-70; T-57 to E-71; R-58 to L-72; L-59 to R-73; R-60 to P-74; L-61 to D-75; H-62 to S-76; A-63 to S-77; F-64 to F-78; D-65 to L-79; Q-66 to A -80; Q-67 to P-81; L-68 to G-82; D-69 to F-83; L-70 to T-84; E-71 to L-85; L-72 to Q-86; R-73 to N-87; P-74 to V-88; D-75 to G-89; S-76 to R-90; S-77 to K -91; F-78 to S-92; L-79 to G-93; A-80 to S-94; P-81 to E-95; G-82 to T-96; F-83 to P-97; T-84 to L-98; L-85 to P-99; Q-86 to E-100; N-87 to T-101; V-88 to D-102; G-89 to L-103; R-90 to A-104; K-91 to H-105; S-92 to C-106; G-93 to F-107; S-94 to Y-108; E-95 to S-109; T-96 to G-110; P-97 to T-111; L-98 to V -112; P-99 to N-113; E-100 to G-114; T-101 to D-115; D-102 to P-116; L-103 to S-117; A-104 to S-118; H-105 to A-119; C-106 to A-120; F-107 to A-121; Y-108 to L-122; S-109 to S-123; G-110 to L-124; T-111 to C-125; V-112 to E-126; N-113 to G-127; G-114 to V-128; D-115 to R-129; P-116 to G-130; S-117 to A-131; S-118 to F-132; A-119 to Y-133; A-120 to L-134; A-121 to L-135; L-122 to G-136; S-123 to E-137; L-124 to A-138; C-125 to Y-139; E-126 to F-140; G-127 to I-141; V-128 to Q-142; R-129 to P-143; G-130 to L-144; A-131 to P-145; F-132 to A-146; Y-133 to A-147; L-134 to S-148; L-135 to E-149; G-136 to R-150; E-137 to L-151; A-138 to A-152; Y-139 to T-153; F-140 to A-154; I-141 to A-155; Q-142 to P-156; P-143 to G-157; L-144 to E-158; P-145 to K-159; A-146 to P-160; A-147 to P-161; S-148 to A-162; E-149 to P-163; R-150 to L-164; L-151 to Q-165; A-152 to F-166; T-153 to H-167; A-154 to L-168; A-155 to L-169; P-156 to R-170; G-157 to R-171; E-158 to N-172; K-159 to R-173; P-160 to Q-174; P-161 to G-175; A-162 to D-176; P-163 to V-177; L-164 to G-178; Q-165 to G-179; F-166 to T-180; H-167 to C-181; L-168 to G-182; L-169 to V-183; R-170 to V-184; R-171 to D-185; N-172 to D-186; R-173 to E-187; Q-174 to P-188; G-175 to R-189; D-176-to P-190; V-177 to T-191; G-178 to G-192; G-179 to K-193; T-180 to A-194; C-181 to E-195; G-182 to T-196; V-183 to E-197; V-184 to D-198; D-185 to E-199; D-186 to D-200; E-187 to E-201; P-188 to G-202; R-189 to T-203; P-190 to E-204; T-191 to G-205; G-192 to E-206; K-193 to D-207; A-194 to E-208; E-195 to G-209; T-196 to P-210; E-197 to Q-211; D-198 to W-212; E-199 to S-213; D-200 to P-214; E-201 to Q-215; G-202 to D-216; T-203 to P-217; E-204 to A-218; G-205 to L-219; E-206 to Q-220; D-207 to G-221; E-208 to V-222; G-209 to G-223; P-210 to Q-224; Q-211 to P-225; W-212 to T-226; S-213 to G-227; P-214 to T-228; Q-215 to G-229; D-216 to S-230; P-217 to I-231; A-218 to R-232; L-219 to K-233; Q-220 to K-234; G-221 to R-235; V-222 to F-236; G-223 to V-237; Q-224 to S-238; P-225 to S-239; T-226 to H-240; G-227 to R-241; T-228 to Y-242; G-229 to V-243; S-230 to E-244; I-231 to T-245; R-232 to M-246; K-233 to L-247; K-234 to V-248; R-235 to A-249; F-236 to D-250; V-237 to Q-251; S-238 to S-252; S-239 to M-253; H-240 to A-254; R-241 to E-255; Y-242 to F-256; V-243 to H-257; E-244 to G-258; T-245 to S-259; M-246 to G-260; L-247 to L-261; V-248 to K-262; A-249 to H-263; D-250 to Y-264; Q-251 to L-265; S-252 to L-266; M-253 to T-267; A-254 to L-268; E-255 to F-269; F-256 to S-270; H-257 to V-271; G-258 to A-272; S-259 to A-273; G-260 to R-274; L-261 to L-275; K-262 to Y-276; H-263 to K-277; Y-264 to H-278; L-265 to P-279; L-266 to S-280; T-267 to I-281; L-268 to R-282; F-269 to N-283; S-270 to S-284; V-271 to V-285; A-272 to S-286; A-273 to L-287; R-274 to V-288; L-275 to V-289; Y-276 to V-290; K-277 to K-291; H-278 to I-292; P-279 to L-293; S-280 to V-294; I-281 to I-295; R-282 to H-296; N-283 to D-297; S-284 to E-298; V-285 to Q-299; S-286 to K-300; L-287 to G-301; V-288 to P-302; V-289 to E-303; V-290 to V-304; K-291 to T-305; I-292 to S-306; L-293 to N-307; V-294 to A-308; I-295 to A-309; H-296 to L-310; D-297 to T-311; E-298 to L-312; Q-299 to R-313; K-300 to N-314; G-301 to F-315; P-302 to C-316; E-303 to N-317; V-304 to W-318; T-305 to Q-319; S-306 to K-320; N-307 to Q-321; A-308 to H-322; A-309 to N-323; L-310 to P-324; T-311 to P-325; L-312 to S-326; R-313 to D-327; N-314 to R-328; F-315 to D-329; C-316 to A-330; N-317 to E-331; W-318 to H-332; Q-319 to Y-333; K-320 to D-334; Q-321 to T-335; H-322 to A-336; N-323 to I-337; P-324 to L-338; P-325 to F-339; S-326 to T-340; D-327 to R-341; R-328 to Q-342; D-329 to D-343; A-330 to L-344; E-331 to C-345; H-332 to G-346; Y-333 to S-347; D-334 to Q-348; T-335 to T-349; A-336 to C-350; I-337 to D-351; L-338 to T-352; F-339 to L-353; T-340 to G-354; R-341 to M-355; Q-342 to A-356; D-343 to D-357; L-344 to V-358; C-345 to G-359; G-346 to T-360; S-347 to V-361; Q-348 to C-362; T-349 to D-363; C-350 to P-364; D-351 to S-365; T-352 to R-366; L-353 to S-367; G-354 to C-368; M-355 to S-369; A-356 to V-370; D-357 to I-371; V-358 to E-372; G-359 to D-373; T-360 to D-374; V-361 to G-375; C-362 to L-376; D-363 to Q-377; P-364 to A-378; S-365 to A-379; R-366 to F-380; S-367 to T-381; C-368 to T-382; S-369 to A-383; V-370 to H-384; I-371 to E-385; E-372 to L-386; D-373 to G-387; D-374 to H-388; G-375 to V-389; L-376 to F-390; Q-377 to N-391; A-378 to M-392; A-379 to P-393; F-380 to H-394; T-381 to D-395; T-382 to D-396; A-383 to A-397; H-384 to K-398; E-385 to Q-399; L-386 to C-400; G-387 to A-401; H-388 to S-402; V-389 to L-403; F-390 to N-404; N-391 to G-405; M-392 to V-406; P-393 to N-407; H-394 to Q-408; D-395 to D-409; D-396 to S-410; A-397 to H-411; K-398 to M-412; Q-399 to M-413; C-400 to A-414; A-401 to S-415; S-402 to M-416; L-403 to L-417; N-404 to S-418; G-405 to N-419; V-406 to L-420; N-407 to D-421; Q-408 to H-422; D-409 to S-423; S-410 to Q-424; H-411 to P-425; M-412 to W-426; M-413 to S-427; A-414 to P-428; S-415 to C-429; M-416 to S-430; L-417 to A-431; S-418 to Y-432; N-419 to M-433; L-420 to I-434; D-421 to T-435; H-422 to S-436; S-423 to F-437; Q-424 to L-438; P-425 to D-439; W-426 to N-440; S-427 to G-441; P-428 to H-442; C-429 to G-443; S-430 to E-444; A-431 to C-445; Y-432 to L-446; M-433 to M-447; 1434 to D-448; T-435 to K-449; S-436 to P-450; F-437 to Q-451; L-438 to N-452; D-439 to P-453; N-440 to I-454; G-441 to Q-455; H-442 to L-456; G-443 to P-457; E-444 to G-458; C-445 to D-459; L-446 to L-460; M-447 to P-461; D-448 to G-462; K-449 to T-463; P-450 to S-464; Q-451 to Y-465; N-452 to D-466; P-453 to A-467; I-454 to N-468; Q-455 to R-469; L-456 to Q-470; P-457 to C-471; G-458 to Q-472; D-459 to F-473; L-460 to T-474; P-461 to F-475; G-462 to G-476; T-463 to E-477; S-464 to D-478; Y-465 to S-479; D-466 to K-480; A-467 to H-481; N-468 to C-482; R-469 to P-483; Q-470 to D-484; C-471 to A-485; Q-472 to A-486; F-473 to S-487; T-474 to T-488; F-475 to C-489; G-476 to S-490; E-477 to T-491; D-478 to L-492; S-479 to W-493; K-480 to C-494; H-481 to T-495; C-482 to G-496; P-483 to T-497; D-484 to S-498; A-485 to G-499; A-486 to G-500; S-487 to V-501; T-488 to L-502; C-489 to V-503; S-490 to C-504; T-491 to Q-505; L-492 to T-506; W-493 to K-507; C-494 to H-508; T-495 to F-509; G-496 to P-510; T-497 to W-511; S-498 to A-512; G-499 to D-513; G-500 to G-514; V-501 to T-515; L-502 to S-516; V-503 to C-517; C-504 to G-518; Q-505 to E-519; T-506 to G-520; K-507 to K-521; H-508 to W-522; F-509 to C-523; P-510 to I-524; W-511 to N-525; A-512 to G-526; D-513 to K-527; G-514 to C-528; T-515 to V-529; S-516 to N-530; C-517 to K-531; G-518 to T-532; E-519 to D-533; G-520 to R-534; K-521 to K-535; W-522 to H-536; C-523 to F-537; I-524 to D-538; N-525 to T-539; G-526 to P-540; K-527 to F-541; C-528 to H-542; V-529 to G-543; N-530 to S-544; K-531 to W-545; T-532 to G-546; D-533 to M-547; R-534 to W-548; K-535 to G-549; H-536 to P-550; F-537 to W-551; D-538 to G-552; T-539 to D-553; P-540 to C-554; F-541 to S-555; H-542 to R-556; G-543 to T-557; S-544 to C-558; W-545 to G-559; G-546 to G-560; M-547 to G-561; W-548 to V-562; G-549 to Q-563; P-550 to Y-564; W-551 to T-565; G-552 to M-566; D-553 to R-567; C-554 to E-568; S-555 to C-569; R-556 to D-570; T-557 to N-571; C-558 to P-572; G-559 to V-573; G-560 to P-574; G-561.to K-575; V-562 to N-576; Q-563 to G-577; Y-564 to G-578; T-565 to K-579; M-566 to Y-580; R-567 to C-581; E-568 to E-582; C-569 to G-583; D-570 to K-584; N-571 to R-585; P-572 to V-586; V-573 to R-587; P-574 to Y-588; K-575 to R-589; N-576 to S-590; G-577 to C-591; G-578 to N-592; K-579 to L-593; Y-580 to E-594; C-581 to D-595; E-582 to C-596; G-583 to P-597; K-584 to D-598; R-585 to N-599; V-586 to N-600; R-587 to G-601; Y-588 to K-602; R-589 to T-603; S-590 to F-604; C-591 to R-605; N-592 to E-606; L-593 to E-607; E-594 to Q-608; D-595 to C-609; C-596 to E-610; P-597 to A-611; D-598 to H-612; N-599 to N-613; N-600 to E-614; G-601 to F-615; K-602 to S-616; T-603 to K-617; F-604 to A-618; R-605 to S-619; E-606 to F-620; E-607 to G-621; Q-608 to S-622; C-609 to G-623; E-610 to P-624; A-611 to A-625; H-612 to V-626; N-613 to E-627; E-614 to W-628; F-615 to I-629; S-616 to P-630; K-617 to K-631; A-618 to Y-632; S-619 to A-633; F-620 to G-634; G-621 to V-635; S-622 to S-636; G-623 to P-637; P-624 to K-638; A-625 to D-639; V-626 to R-640; E-627 to C-641; W-628 to K-642; I-629 to L-643; P-630 to I-644; K-631 to C-645; Y-632 to Q-646; A-633 to A-647; G-634 to K-648; V-635 to G-649; S-636 to I-650; P-637 to G-651; K-638 to Y-652; D-639 to F-653; R-640 to F-654; C-641 to V-655; K-642 to L-656; L-643 to Q-657; I-644 to P-658; C-645 to K-659; Q-646 to V-660; A-647 to V-661; K-648 to D-662; G-649 to G-663; I-650 to T-664; G-651 to P-665; Y-652 to C-666; F-653 to S-667; F-654 to P-668; V-655 to D-669; L-656 to S-670; Q-657 to T-671; P-658 to S-672; K-659 to V-673; V-660 to C-674; V-661 to V-675; D-662 to Q-676; G-663 to G-677; T-664 to Q-678; P-665 to C-679; C-666 to V-680; S-667 to K-681; P-668 to A-682; D-669 to G-683; S-670 to C-684; T-671 to D-685; S-672 to R-686; V-673 to I-687; C-674 to I-688; V-675 to D-689; Q-676 to S-690; G-677 to K-691; Q-678 to K-692; C-679 to K-693; V-680 to F-694; K-681 to D-695; A-682 to K-696; G-683 to C-697; C-684 to G-698; D-685 to V-699; R-686 to C-700; I-687 to G-701; I-688 to G-702; D-689 to N-703; S-690 to G-704; K-691 to S-705; K-692 to T-706; K-693 to C-707; F-694 to K-708; D-695 to K-709; K-696 to I-710; C-697 to S-711; G-698 to G-712; V-699 to S-713; C-700 to V-714; G-701 to T-715; G-702 to S-716; N-703 to A-717; G-704 to K-718; S-705 to P-719; T-706 to G-720; C-707 to Y-721; K-708 to H-722; K-709 to D-723; I-710 to I-724; S-711 to I-725; G-712 to T-726; S-713 to I-727; V-714 to P-728; T-715 to T-729; S-716 to G-730; A-717 to A-731; K-718 to T-732; P-719 to N-733; G-720 to I-734; Y-721 to E-735; H-722 to V-736; D-723 to K-737; I-724 to Q-738; I-725 to R-739; T-726 to N-740; I-727 to Q-741; P-728 to R-742; T-729 to G-743; G-730 to S-744; A-731 to R-745; T-732 to N-746; N-733 to N-747; I-734 to G-748; E-735 to S-749; V-736 to F-750; K-737 to L-751; Q-738 to A-752; R-739 to I-753; N-740 to K-754; Q-741 to A-755; R-742 to A-756; G-743 to D-757; S-744 to G-758; R-745 to T-759; N-746 to Y-760; N-747 to I-761; G-748 to L-762; S-749 to N-763; F-750 to G-764; L-751 to D-765; A-752 to Y-766; I-753 to T-767; K-754 to L-768; A-755 to S-769; A-756 to T-770; D-757 to L-771; G-758 to E-772; T-759 to Q-773; Y-760 to D-774; I-761 to I-775; L-762 to M-776; N-763 to Y-777; G-764 to K-778; D-765 to G-779; Y-766 to V-780; T-767 to V-781; L-768 to L-782; S-769 to R-783; T-770 to Y-784; L-771 to S-785; E-772 to G-786; Q-773 to S-787; D-774 to S-788; I-775 to A-789; M-776 to A-790; Y-777 to L-791; K-778 to E-792; G-779 to R-793; V-780 to I-794; V-781 to R-795; L-782 to S-796; R-783 to F-797; Y-784 to S-798; S-785 to P-799; G-786 to L-800; S-787 to K-801; S-788 to E-802; A-789 to P-803; A-790 to L-804; L-791 to T-805; E-792 to I-806; R-793 to Q-807; I-794 to V-808; R-795 to L-809; S-796 to T-810; F-797 to V-811; S-798 to G-812; P-799 to N-813; L-800 to A-814; K-801 to L-815; E-802 to R-816; P-803 to P-817; L-804 to K-818; T-805 to I-819; I-806 to K-820; Q-807 to Y-821; V-808 to T-822; L-809 to Y-823; T-810 to F-824; V-811 to V-825; G-812 to K-826; N-813 to K-827; A-814 to K-828; L-815 to K-829; R-816 to E-830; P-817 to S-831; K-818 to F-832; I-819 to N-833; K-820 to A-834; Y-821 to I-835; T-822 to P-836; Y-823 to T-837; F-824 to F-838; V-825 to S-839; K-826 to A-840; K-827 to W-841; K-828 to V-842; K-829 to I-843; E-830 to E-844; S-831 to E-845; F-832 to W-846; N-833 to G-847; A-834 to E-848; I-835 to C-849; P-836 to S-850; T-837 to K-851; F-838 to S-852; S-839 to C-853; A-840 to E-854; W-841 to L-855; V-842 to G-856; I-843 to W-857; E-844 to Q-858; E-845 to R-859; W-846 to R-860; G-847 to L-861; E-848 to V-862; C-849 to E-863; S-850 to C-864; K-851 to R-865; S-852 to D-866; C-853 to I-867; E-854 to N-868; L-855 to G-869; G-856 to Q-870; W-857 to P-871; Q-858 to A-872; R-859 to S-873; R-860 to E-874; L-861 to C-875; V-862 to A-876; E-863 to K-877; C-864 to E-878; R-865 to V-879; D-866 to K-880; I-867 to P-881; N-868 to A-882; G-869 to S-883; Q-870 to T-884; P-871 to R-885; A-872 to P-886; S-873 to C-887; E-874 to A-888; C-875 to D-889; A-876 to H-890; K-877 to P-891; E-878 to C-892; V-879 to P-893; K-880 to Q-894; P-881 to W-895; A-882 to Q-896; S-883 to L-897; T-884 to G-898; R-885 to E-899; P-886 to W-900; C-887 to S-901; A-888 to S-902; D-889 to C-903; H-890 to S-904; P-891 to K-905; C-892 to T-906; P-893 to C-907; Q-894 to G-908; W-895 to K-909; Q-896 to G-910; L-897 to Y-911; G-898 to K-912; E-899 to K-913; W-900 to R-914; S-901 to S-915; S-902 to L-916; C-903 to K-917; S-904 to C-918; K-905 to L-919; T-906 to S-920; C-907 to H-921; G-908 to D-922; K-909 to G-923; G-910 to G-924; Y-911 to V-925; K-912 to L-926; K-913 to S-927; R-914 to H-928; S-915 to E-929; L-916 to S-930; K-917 to C-931; C-918 to D-932; L-919 to P-933; S-920 to L-934; H-921 to K-935; D-922 to K-936; G-923 to P-937; G-924 to K-938; V-925 to H-939; L-926 to F-940; S-927 to I-941; H-928 to D-942; E-929 to F-943; S-930 to C-944; C-931 to T-945; D-932 to M-946; P-933 to A-947; L-934 to E-948; K-935 to C-949; and/or K-936 to S-950 of SEQ ID NO:2.

Similarly, preferred antigenic epitopes of METH2 comprise, or alternatively consist of, the amino acid sequence of residues: M-1 to L-15; F-2 to L-16; P-3 to L-17; A-4 to L-18; P-5 to L-19; A-6 to L-20; A-7 to L-21; P-8 to L-22; R-9 to P-23; W-10 to L-24; L-11 to A-25; P-12 to R-26; F-13 to G-27; L-14 to A-28; L-15 to P-29; L-16 to A-30; L-17 to R-31; L-18 to P-32; L-19 to A-33; L-20 to A-34; L-21 to G-35; L-22 to G-36; P-23 to Q-37; L-24 to A-38; A-25 to S-39; R-26 to E-40; G-27 to L-41; A-28 to V-42; P-29 to V-43; A-30 to P-44; R-31 to T-45; P-32 to R-46; A-33 to L-47; A-34 to P-48; G-35 to G-49; G-36 to S-50; Q-37 to A-51; A-38 to G-52; S-39 to E-53; E-40 to L-54; L-41 to A-55; V-42 to L-56; V-43 to H-57; P-44 to L-58; T-45 to S-59; R-46 to A-60; L-47 to F-61; P-48 to G-62; G-49 to K-63; S-50 to G-64; A-51 to F-65; G-52 to V-66; E-53 to L-67; L-54 to R-68; A-55 to L-69; L-56 to A-70; H-57 to P-71; L-58 to D-72; S-59 to D-73; A-60 to S-74; F-61 to F-75; G-62 to L-76; K-63 to A-77; G-64 to P-78; F-65 to E-79; V-66 to F-80; L-67 to K-81; R-68 to I-82; L-69 to E-83; A-70 to R-84; P-71 to L-85; D-72 to G-86; D-73 to G-87; S-74 to S-88; F-75 to G-89; L-76 to R-90; A-77 to A-91; P-78 to T-92; E-79 to G-93; F-80 to G-94; K-81 to E-95; I-82 to R-96; E-83 to G-97; R-84 to L-98; L-85 to R-99; G-86 to G-100; G-87 to C-101; S-88 to F-102; G-89 to F-103; R-90 to S-104; A-91 to G-105; T-92 to T-106; G-93 to V-107; G-94 to N-108; E-95 to G-109; R-96 to E-110; G-97 to P—I-11; L-98 to E-112; R-99 to S-113; G-100 to L-114; C-101 to A-115; F-102 to A-116; F-103 to V-117; S-104 to S-118; G-105 to L-119; T-106 to C-120; V-107 to R-121; N-108 to G-122; G-109 to L-123; E-110 to S-124; P-111 to G-125; E-112 to S-126; S-113 to F-127; L-114 to L-128; A-115 to L-129; A-116 to D-130; V-117 to G-131; S-118 to E-132; L-119 to E-133; C-120 to F-134; R-121 to T-135; G-122 to I-136; L-123 to Q-137; S-124 to P-138; G-125 to Q-139; S-126 to G-140; F-127 to A-141; L-128 to G-142; L-129 to G-143; D-130 to S-144; G-131 to L-145; E-132 to A-146; E-133 to Q-147; F-134 to P-148; T-135 to H-149; I-136 to R-150; Q-137 to L-151; P-138 to Q-152; Q-139 to R-153; G-140 to W-154; A-141 to G-155; G-142 to P-156; G-143 to A-157; S-144 to G-158; L-145 to A-159; A-146 to R-160; Q-147 to P-161; P-148 to L-162; H-149 to P-163; R-150 to R-164; L-151 to G-165; Q-152 to P-166; R-153 to E-167; W-154 to W-168; G-155 to E-169; P-156 to V-170; A-157 to E-171; G-158 to T-172; A-159 to G-173; R-160 to E-174; P-161 to G-175; L-162 to Q-176; P-163 to R-177; R-164 to Q-178; G-165 to E-179; P-166 to R-180; E-167 to G-181; W-168 to D-182; E-169 to H-183; V-170 to Q-184; E-171 to E-185; T-172 to D-186; G-173 to S-187; E-174 to E-188; G-175 to E-189; Q-176 to E-190; R-177 to S-191; Q-178 to Q-192; E-179 to E-193; R-180 to E-194; G-181 to E-195; D-182 to A-196; H-183 to E-197; Q-184 to G-198; E-185 to A-199; D-186 to S-200; S-187 to E-201; E-188 to P-202; E-189 to P-203; E-190 to P-204; S-191 to P-205; Q-192 to L-206; E-193 to G-207; E-194 to A-208; E-195 to T-209; A-196 to S-210; E-197 to R-211; G-198 to T-212; A-199 to K-213; S-200 to R-214; E-201 to F-215; P-202 to V-216; P-203 to S-217; P-204 to E-218; P-205 to A-219; L-206 to R-220; G-207 to F-221; A-208 to V-222; T-209 to E-223; S-210 to T-224; R-211 to L-225; T-212 to L-226; K-213 to V-227; R-214 to A-228; F-215 to D-229; V-216 to A-230; S-217 to S-231; E-218 to M-232; A-219 to A-233; R-220 to A-234; F-221 to F-235; V-222 to Y-236; E-223 to G-237; T-224 to A-238; L-225 to D-239; L-226 to L-240; V-227 to Q-241; A-228 to N-242; D-229 to H-243; A-230 to I-244; S-231 to L-245; M-232 to T-246; A-233 to L-247; A-234 to M-248; F-235 to S-249; Y-236 to V-250; G-237 to A-251; A-238 to A-252; D-239 to R-253; L-240 to I-254; Q-241 to Y-255; N-242 to K-256; H-243 to H-257; I-244 to P-258; L-245 to S-259; T-246 to I-260; L-247 to K-261; M-248 to N-262; S-249 to S-263; V-250 to I-264; A-251 to N-265; A-252 to L-266; R-253 to M-267; I-254 to V-268; Y-255 to V-269; K-256 to K-270; H-257 to V-271; P-258 to L-272; S-259 to I-273; I-260 to V-274; K-261 to E-275; N-262 to D-276; S-263 to E-277; I-264 to K-278; N-265 to W-279; L-266 to G-280; M-267 to P-281; V-268 to E-282; V-269 to V-283; K-270 to S-284; V-271 to D-285; L-272 to N-286; I-273 to G-287; V-274 to G-288; E-275 to L-289; D-276 to T-290; E-277 to L-291; K-278 to R-292; W-279 to N-293; G-280 to F-294; P-281 to C-295; E-282 to N-296; V-283 to W-297; S-284 to Q-298; D-285 to R-299; N-286 to R-300; G-287 to F-301; G-288 to N-302; L-289 to Q-303; T-290 to P-304; L-291 to S-305; R-292 to D-306; N-293 to R-307; F-294 to H-308; C-295 to P-309; N-296 to E-310; W-297 to H-311; Q-298 to Y-312; R-299 to D-313; R-300 to T-314; F-301 to A-315; N-302 to I-316; Q-303 to L-317; P-304 to L-318; S-305 to T-319; D-306 to R-320; R-307 to Q-321; H-308 to N-322; P-309 to F-323; E-310 to C-324; H-311 to G-325; Y-312 to Q-326; D-313 to E-327; T-314 to G-328; A-315 to L-329; I-316 to C-330; L-317 to D-331; L-318 to T-332; T-319 to L-333; R-320 to G-334; Q-321 to V-335; N-322 to A-336; F-323 to D-337; C-324 to I-338; G-325 to G-339; Q-326 to T-340; E-327 to I-341; G-328 to C-342; L-329 to D-343; C-330 to P-344; D-331 to N-345; T-332 to K-346; L-333 to S-347; G-334 to C-348; V-335 to S-349; A-336 to V-350; D-337 to I-351; I-338 to E-352; G-339 to D-353; T-340 to E-354; I-341 to G-355; C-342 to L-356; D-343 to Q-357; P-344 to A-358; N-345 to A-359; K-346 to H-360; S-347 to T-361; C-348 to L-362; S-349 to A-363; V-350 to H-364; I-351 to E-365; E-352 to L-366; D-353 to G-367; E-354 to H-368; G-355 to V-369; L-356 to L-370; Q-357 to S-371; A-358 to M-372; A-359 to P-373; H-360 to H-374; T-361 to D-375; L-362 to D-376; A-363 to S-377; H-364 to K-378; E-365 to P-379; L-366 to C-380; G-367 to T-381; H-368 to R-382; V-369 to L-383; L-370 to F-384; S-371 to G-385; M-372 to P-386; P-373 to M-387; H-374 to G-388; D-375 to K-389; D-376 to H-390; S-377 to H-391; K-378 to V-392; P-379 to M-393; C-380 to A-394; T-381 to P-395; R-382 to L-396; L-383 to F-397; F-384 to V-398; G-385 to H-399; P-386 to L-400; M-387 to N-401; G-388 to Q-402; K-389 to T-403; H-390 to L-404; H-391 to P-405; V-392 to W-406; M-393 to S-407; A-394 to P-408; P-395 to C-409; L-396 to S-410; F-397 to A-411; V-398 to M-412; H-399 to Y-413; L-400 to L-414; N-401 to T-415; Q-402 to E-416; T-403 to L-417; L-404 to L-418; P-405 to D-419; W-406 to G-420; S-407 to G-421;

P-408 to H-422; C-409 to G-423; S-410 to D-424; A-411 to C-425; M-412 to L-426; Y-413 to L-427; L-414 to D-428; T-415 to A-429; E-416 to P-430; L-417 to G-431; L-418 to A-432; D-419 to A-433; G-420 to L-434; G-421 to P-435; H-422 to L-436; G-423 to P-437; D-424 to T-438; C-425 to G-439; L-426 to L-440; L-427 to P-441; D-428 to G-442; A-429 to R-443; P-430 to M-444; G-431 to A-445; A-432 to L-446; A-433 to Y-447;

L-434 to Q-448; P-435 to L-449; L-436 to D-450; P-437 to Q-451; T-438 to Q-452; G-439 to C-453; L-440 to R-454; P-441 to Q-455; G-442 to 1456; R-443 to F-457; M-444 to G-458; A-445 to P-459; L-446 to D-460; Y-447 to F-461; Q-448 to R-462; L-449 to H-463; D-450 to C-464; Q-451 to P-465; Q-452 to N-466; C-453 to T-467; R-454 to S-468; Q-455 to A-469; I-456 to Q-470; F-457 to D-471; G-458 to V-472; P-459 to C-473;

D-460 to A-474; F-461 to Q-475; R-462 to L-476; H-463 to W-477; C-464 to C-478; P-465 to H-1479; N-466 to T-480; T-467 to D-481; S-468 to G-482; A-469 to A-483; Q-470 to E-484; D-471 to P-485; V-472 to L-486; C-473 to C-487; A-474 to H-488; Q-475 to T-489; L-476 to K-490; W-477 to N-491; C-478 to G-492; H-479 to S-493; T-480 to L-494; D-481 to P-495; G-482 to W-496; A-483 to A-497; E-484 to D-498; P-485 to G-499; L-486 to T-500; C-487 to P-501; H-1488 to C-502; T-489 to G-503; K-490 to P-504;

N-491 to G-505; G-492 to H-506; S-493 to L-507; L-494 to C-508; P-495 to S-509; W-496 to E-510; A-497 to G-511; D-498 to S-512; G-499 to C-513; T-500 to L-514; P-501 to P-515; C-502 to E-516; G-503 to E-517; P-504 to E-518; G-505 to V-519; H-506 to E-520; L-507 to R-521; C-508 to P-522; S-509 to K-523; E-510 to P-524; G-511 to V-525;

S-512 to V-526; C-513 to D-527; L-514 to G-528; P-515 to G-529; E-516 to W-530; E-517 to A-531; E-518 to P-532; V-519 to W-533; E-520 to G-534; R-521 to P-535; P-522 to W-536; K-523 to G-537; P-524 to E-538; V-525 to C-539; V-526 to S-540; D-527 to R-541; G-528 to T-542; G-529 to C-543; W-530 to G-544; A-531 to G-545; P-532 to G-546; W-533 to V-547; G-534 to Q-548; P-535 to F-549; W-536 to S-550; G-537 to H-551; E-538 to R-552; C-539 to E-553; S-540 to C-554; R-541 to K-555; T-542 to D-556;

C-543 to P-557; G-544 to E-558; G-545 to P-559; G-546 to Q-560; V-547 to N-561; Q-548 to G-562; F-549 to G-563; S-550 to R-564; H-551 to Y-565; R-552 to C-566; E-553 to L-567; C-554 to G-568; K-555 to R-569; D-556 to R-570; P-557 to A-571; E-558 to 30 K-572; P-559 to Y-573; Q-560 to Q-574; N-561 to S-575; G-562 to C-576; G-563 to H-577; R-564 to T-578; Y-565 to E-579; C-566 to E-580; L-567 to C-581; G-568 to P-582; R-569 to P-583; R-570 to D-584; A-571 to G-585; K-572 to K-586; Y-573 to S-587; Q-574 to F-588; S-575 to R-589; C-576 to E-590; H-577 to Q-591; T-578 to Q-592; E-579 to C-593; E-580 to E-594; C-581 to K-595; P-582 to Y-596; P-583 to N-597; D-584 to A-598; G-585 to Y-599; K-586 to N-600; S-587 to Y-601; F-588 to T-602; R-589 to D-603; E-590 to M-604; Q-591 to D-605; Q-592 to G-606; C-593 to N-607; E-594 to L-608; K-595 to L-609; Y-596 to Q-610; N-597 to W-611; A-598 to V-612; Y-599 to P-613; N-600 to K-614; Y-601 to Y-615; T-602 to A-616; D-603 to G-617; M-604 to V-618; D-605 to S-619; G-606 to P-620; N-607 to R-621; L-608 to D-622; L-609 to R-623; Q-610 to C-624; W-611 to K-625; V-612 to L-626; P-613 to F-627; K-614 to C-628; Y-615 to R-629; A-616 to A-630; G-617 to R-631; V-618 to G-632; S-619 to R-633; P-620 to S-634; R-621 to E-635; D-622 to F-636; R-623 to K-637; C-624 to V-638; K-625 to F-639; L-626 to E-640; F-627 to A-641; C-628 to K-642; R-629 to V-643; A-630 to I-644; R-631 to D-645; G-632 to G-646; R-633 to T-647; S-634 to L-648; E-635 to C-649; F-636 to G-650; K-637 to P-651; V-638 to E-652; F-639 to T-653; E-640 to L-654; A-641 to A-655; K-642 to I-656; V-643 to C-657; I-644 to V-658; D-645 to R-659; G-646 to G-660; T-647 to Q-661; L-648 to C-662; C-649 to V-663; G-650 to K-664; P-651 to A-665; E-652 to G-666; T-653 to C-667; L-654 to D-668; A-655 to H-669; I-656 to V-670; C-657 to V-671; V-658 to D-672; R-659 to S-673; G-660 to P-674; Q-661 to R-675; C-662 to K-676; V-663 to L-677; K-664 to D-678; A-665 to K-679; G-666 to C-680; C-667 to G-681; D-668 to V-682; H-669 to C-683; V-670 to G-684; V-671 to G-685; D-672 to K-686; S-673 to G-687; P-674 to N-688; R-675 to S-689; K-676 to C-690; L-677 to R-691; D-678 to K-692; K-679 to V-693; C-680 to S-694; G-681 to G-695; V-682 to S-696; C-683 to L-697; G-684 to T-698; G-685 to P-699; K-686 to T-700; G-687 to N-701; N-688 to Y-702; S-689 to G-703; C-690 to Y-704; R-691 to N-705; K-692 to D-706; V-693 to I-707; S-694 to V-708; G-695 to T-709; S-696 to I-710; L-697 to P-711; T-698 to A-712; P-699 to G-713; T-700 to A-714; N-701 to T-715; Y-702 to N-716; G-703 to I-717; Y-704 to D-718; N-705 to V-719; D-706 to K-720; I-707 to Q-721; V-708 to R-722; T-709 to S-723; I-710 to H-724; P-711 to P-725; A-712 to G-726; G-713 to V-727; A-714 to Q-728; T-715 to N-729; N-716 to D-730; I-717 to G-731; D-718 to N-732; V-719 to Y-733; K-720 to L-734; Q-721 to A-735; R-722 to L-736; S-723 to K-737; H-724 to T-738; P-725 to A-739; G-726 to D-740; V-727 to G-741; Q-728 to Q-742; N-729 to Y-743; D-730 to L-744; G-731 to L-745; N-732 to N-746; Y-733 to G-747; L-734 to N-748; A-735 to L-749; L-736 to A-750; K-737 to I-751; T-738 to S-752; A-739 to A-753; D-740 to I-754; G-741 to E-755; Q-742 to Q-756; Y-743 to D-757; L-744 to I-758; L-745 to L-759; N-746 to V-760; G-747 to K-761; N-748 to G-762; L-749 to T-763; A-750 to I-764; I-751 to L-765; S-752 to K-766; A-753 to Y-767; I-754 to S-768; E-755 to G-769; Q-756 to S-770; D-757 to I-771; I-758 to A-772; L-759 to T-773; V-760 to L-774; K-761 to E-775; G-762 to R-776; T-763 to L-777; I-764 to Q-778; L-765 to S-779; K-766 to F-780; Y-767 to R-781; S-768 to P-782; G-769 to L-783; S-770 to P-784; I-771 to E-785; A-772 to P-786; T-773 to L-787; L-774 to T-788; E-775 to V-789; R-776 to Q-790; L-777 to L-791; Q-778 to L-792; S-779 to T-793; F-780 to V-794; R-781 to P-795; P-782 to G-796; L-783 to E-797; P-784 to V-798; E-785 to F-799; P-786 to P-800; L-787 to P-801; T-788 to K-802; V-789 to V-803; Q-790 to K-804; L-791 to Y-805; L-792 to T-806; T-793 to F-807; V-794 to F-808; P-795 to V-809; G-796 to P-810; E-797 to N-811; V-798 to D-812; F-799 to V-813; P-800 to D-814; P-801 to F-815; K-802 to S-816; V-803 to M-817; K-804 to Q-818; Y-805 to S-819; T-806 to S-820; F-807 to K-821; F-808 to E-822; V-809 to R-823; P-810 to A-824; N-811 to T-825; D-812 to T-826; V-813 to N-827; D-814 to I-828; F-815 to I-829; S-816 to Q-830; M-817 to P-831; Q-818 to L-832; $-819 to L-833; S-820 to H-834; K-821 to A-835; E-822 to Q-836; R-823 to W-837; A-824 to V-838; T-825 to L-839; T-826 to G-840; N-827 to D-841; I-828 to W-842; I-829 to S-843; Q-830 to E-844; P-831 to C-845; L-832 to S-846; L-833 to S-847; H-834 to T-848; A-835 to C-849; Q-836 to G-850; W-837 to A-851; V-838 to G-852; L-839 to W-853; G-840 to Q-854; D-841 to R-855; W-842 to R-856; S-843 to T-857; E-844 to V-858; C-845 to E-859; S-846 to C-860; S-847 to R-861; T-848 to D-862; C-849 to P-863; G-850 to S-864; A-851 to G-865; G-852 to Q-866; W-853 to A-867; Q-854 to S-868; R-855 to A-869; R-856 to T-870; T-857 to C-871; V-858 to N-872; E-859 to K-873; C-860 to A-874; R-861 to L-875; D-862 to K-876; P-863 to P-877; S-864 to E-878; G-865 to D-879; Q-866 to A-880; A-867 to K-881; S-868 to P-882; A-869 to C-883; T-870 to E-884; C-871 to S-885; N-872 to Q-886; K-873 to L-887; A-874 to C-888; L-875 to P-889; and/or K-876 to L-890 of SEQ ID NO:4.

Polynucleotides encoding these polypeptide fragments are also encompassed by the invention.

Additional non-exclusive preferred antigenic epitopes include the antigenic epitopes disclosed herein, as well as portions thereof. Antigenic epitopes are useful, for example, to raise antibodies, including monoclonal antibodies, that specifically bind the epitope. Preferred antigenic epitopes include the antigenic epitopes disclosed herein, as well as any combination of two, three, four, five or more of these antigenic epitopes. Antigenic epitopes can be used as the target molecules in immunoassays. (See, for instance, Wilson et al., Cell 37:767-778 (1984); Sutcliffe et al., Science 219:660-666 (1983)).

Similarly, immunogenic epitopes can be used, for example, to induce antibodies according to methods well known in the art. (See, for instance, Sutcliffe et al., supra; Wilson et al., supra; Chow et al., Proc. Natl. Acad. Sci. USA 82:910-914; and Bittle et al., J. Gen. Virol. 66:2347-2354 (1985). Preferred immunogenic epitopes include the immunogenic epitopes disclosed herein, as well as any combination of two, three, four, five or more of these immunogenic epitopes. The polypeptides comprising one or more immunogenic epitopes may be presented for eliciting an antibody response together with a carrier protein, such as an albumin, to an animal system (such as rabbit or mouse), or, if the polypeptide is of sufficient length (at least about 25 amino acids), the polypeptide may be presented without a carrier. However, immunogenic epitopes comprising as few as 8 to 10 amino acids have been shown to be sufficient to raise antibodies capable of binding to, at the very least, linear epitopes in a denatured polypeptide (e.g., in Western blotting).

Epitope-bearing polypeptides of the present invention may be used to induce antibodies according to methods well known in the art including, but not limited to, in vivo immunization, in vitro immunization, and phage display methods. See, e.g., Sutcliffe et al., supra; Wilson et al., supra, and Bittle et al., J. Gen. Virol., 66:2347-2354 (1985). If in vivo immunization is used, animals may be immunized with free peptide; however, anti-peptide antibody titer may be boosted by coupling the peptide to a macromolecular carrier, such as keyhole limpet hemacyanin (KLH) or tetanus toxoid. For instance, peptides containing cysteine residues may be coupled to a carrier using a linker such as m-maleimidobenzoyl-N-hydroxysuccinimide ester (MBS), while other peptides may be coupled to carriers using a more general linking agent such as glutaraldehyde. Animals such as rabbits, rats and mice are immunized with either free or carrier-coupled peptides, for instance, by intraperitoneal and/or intradermal injection of emulsions containing about 100 μg of peptide or carrier protein and Freund's adjuvant or any other adjuvant known for stimulating an immune response. Several booster injections may be needed, for instance, at intervals of about two weeks, to provide a useful titer of anti-peptide antibody which can be detected, for example, by ELISA assay using free peptide adsorbed to a solid surface. The titer of anti-peptide antibodies in serum from an immunized animal may be increased by selection of anti-peptide antibodies, for instance, by adsorption to the peptide on a solid support and elution of the selected antibodies according to methods well known in the art.

As one of skill in the art will appreciate, and as discussed above, the polypeptides of the present invention comprising an immunogenic or antigenic epitope can be fused to other polypeptide sequences. For example, the polypeptides of the present invention may be fused with the constant domain of immunoglobulins (IgA, IgE, IgG, IgM), or portions thereof (CH1, CH2, CH3, or any combination thereof and portions thereof) resulting in chimeric polypeptides. Such fusion proteins may facilitate purification and may increase half-life in vivo. This has been shown for chimeric proteins consisting of the first two domains of the human CD4-polypeptide and various domains of the constant regions of the heavy or light chains of mammalian immunoglobulins. See, e.g., EP 394,827; Traunecker et al., Nature, 331:84-86 (1988). Enhanced delivery of an antigen across the epithelial barrier to the immune system has been demonstrated for antigens (e.g., insulin) conjugated to an FcRn binding partner such as IgG or Fc fragments (see, e.g., PCT Publications WO 96/22024 and WO 99/04813). IgG Fusion proteins that have a disulfide-linked dimeric structure due to the IgG portion desulfide bonds have also been found to be more efficient in binding and neutralizing other molecules than monomeric polypeptides or fragments thereof alone. See, e.g., Fountoulakis et al., J. Biochem., 270:3958-3964 (1995). Nucleic acids encoding the above epitopes can also be recombined with a gene of interest as an epitope tag (e.g., the hemagglutinin (“HA”) tag or flag tag) to aid in detection and purification of the expressed polypeptide. For example, a system described by Janknecht et al. allows for the ready purification of non-denatured fusion proteins expressed in human cell lines (Janknecht et al., 1991, Proc. Natl. Acad. Sci. USA 88:8972-897). In this system, the gene of interest is subcloned into a vaccinia recombination plasmid such that the open reading frame of the gene is translationally fused to an amino-terminal tag consisting of six histidine residues. The tag serves as a matrix binding domain for the fusion protein. Extracts from cells infected with the recombinant vaccinia virus are loaded onto Ni2+nitriloacetic acid-agarose column and histidine-tagged proteins can be selectively eluted with imidazole-containing buffers.

Additional fusion proteins of the invention may be generated through the techniques of gene-shuffling, motif-shuffling, exon-shuffling, and/or codon-shuffling (collectively referred to as “DNA shuffling”). DNA shuffling may be employed to modulate the activities of polypeptides of the invention, such methods can be used to generate polypeptides with altered activity, as well as agonists and antagonists of the polypeptides. See, generally, U.S. Pat. Nos. 5,605,793; 5,811,238; 5,830,721; 5,834,252; and 5,837,458, and Patten et al., Curr. Opinion Biotechnol. 8:724-33 (1997); Harayama, Trends Biotechnol. 16(2):76-82 (1998); Hansson, et al., J. Mol. Biol. 287:265-76 (1999); and Lorenzo and Blasco, Biotechniques 24(2):308-13 (1998) (each of these patents and publications are hereby incorporated by reference in its entirety). In one embodiment, alteration of polynucleotides corresponding to SEQ ID NO: 1 or 3 and the polypeptides encoded by these polynucleotides may be achieved by DNA shuffling. DNA shuffling involves the assembly of two or more DNA segments by homologous or site-specific recombination to generate variation in the polynucleotide sequence. In another embodiment, polynucleotides of the invention, or the encoded polypeptides, may be altered by being subjected to random mutagenesis by error-prone PCR, random nucleotide insertion or other methods prior to recombination. In another embodiment, one or more components, motifs, sections, parts, domains, fragments, etc., of a polynucleotide encoding a polypeptide of the invention may be recombined with one or more components, motifs, sections, parts, domains, fragments, etc. of one or more heterologous molecules.

Fusion proteins that have a disulfide-linked dimeric structure due to the IgG portion can also be more efficient in binding and neutralizing other molecules than monomeric polypeptides or fragments thereof alone. See, e.g., Fountoulakis et al. (1995) J. Biochem. 270:3958-3964. Nucleic acids encoding the above epitopes can also be recombined with a gene of interest as an epitope tag to aid in detection and purification of the expressed polypeptide.

The present invention further relates to antibodies and T-cell antigen receptors (TCR) which specifically bind the polypeptides of the present invention. The antibodies of the present invention include IgG (including IgG1, IgG2, IgG3, and IgG4), IgA (including IgA1 and IgA2), IgD, IgE, or IgM, and IgY. As used herein, the term “antibody” (Ab) is meant to include whole antibodies, including single-chain whole antibodies, and antigen-binding fragments thereof. Most preferably the antibodies are human antigen binding antibody fragments of the present invention including, but not limited to, Fab, Fab′ and F(ab′)₂, Fd, single-chain Fvs (scFv), single-chain antibodies, disulfide-linked Fvs (sdfv) and fragments comprising either a VL or VH domain. The antibodies may be from any animal origin including birds and mammals. Preferably, the antibodies are human, murine, rabbit, goat, guinea pig, camel, horse, or chicken.

Antigen-binding antibody fragments, including single-chain antibodies, may comprise the variable region(s) alone or in combination with the entire or partial of the following: hinge region, CH1, CH2, and CH3 domains. Also included in the invention are any combinations of variable region(s) and hinge region, CH1, CH2, and CH3 domains. The present invention further includes monoclonal, polyclonal, chimeric, humanized, and human monoclonal and polyclonal antibodies which specifically bind the polypeptides of the present invention. The present invention further includes antibodies which are anti-idiotypic to the antibodies of the present invention.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for heterologous compositions, such as a heterologous polypeptide or solid support material. See, e.g., WO 93/17715; WO 92/08802; WO 91/00360; WO 92/05793; Tutt, A. et al. (1991) J. Immunol. 147:60-69; U.S. Pat. Nos. 5,573,920, 4,474,893, 5,601,819, 4,714,681, 4,925,648; Kostelny, S. A. et al. (1992) J. Immunol. 148:1547-1553.

Antibodies of the present invention may be described or specified in terms of the epitope(s) or portion(s) of a polypeptide of the present invention which are recognized or specifically bound by the antibody. The epitope(s) or polypeptide portion(s) may be specified as described herein, e.g., by N-terminal and C-terminal positions, by size in contiguous amino acid residues, or listed in the Tables and Figures. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their cross-reactivity. Antibodies that do not bind any other analog, ortholog, or homolog of the polypeptides of the present invention are included. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. Further included in the present invention are antibodies which only bind polypeptides encoded by polynucleotides which hybridize to a polynucleotide of the present invention under stringent hybridization conditions (as described herein). Antibodies of the present invention may also be described or specified in terms of their binding affinity. Preferred binding affinities include those with a dissociation constant or Kd less than 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, and 10⁻¹⁵M.

Antibodies of the present invention have uses that include, but are not limited to, methods known in the art to purify, detect, and target the polypeptides of the present invention including both in vitro and in vivo diagnostic and therapeutic methods. For example, the antibodies have use in immunoassays for qualitatively and quantitatively measuring levels of the polypeptides of the present invention in biological samples. See, e.g., Harlow et al., ANTIBODIES: A LABORATORY MANUAL, (Cold Spring Harbor Laboratory Press, 2nd ed. 1988) (incorporated by reference in the entirety).

The antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387.

The antibodies of the present invention may be prepared by any suitable method known in the art. For example, a polypeptide of the present invention or an antigenic fragment thereof can be administered to an animal in order to induce the production of sera containing polyclonal antibodies. The term “monoclonal antibody” is not limited to antibodies produced through hybridoma technology. The term “monoclonal antibody” refers to an antibody that is derived from a single clone, including eukaryotic, prokaryotic, or phage clones, and not by the method which it is produced. Monoclonal antibodies can be prepared using a wide variety of techniques known in the art including the use of hybridoma, recombinant and phage display technology.

Hybridoma techniques include those known in the art and taught in Harlow et al., ANTIBODIES: A LABORATORY MANUAL, (Cold Spring Harbor Laboratory Press, 2nd ed. 1988); Hammerling, et al., in: MONOCLONAL ANTIBODIES AND T-CELL HYBRIDOMAS 563-681 (Elsevier, N.Y., 1981) (said references incorporated by reference in their entireties). Fab and F(ab′)₂ fragments may be produced by proteolytic cleavage, using enzymes such as papain (to produce Fab fragments) or pepsin (to produce F(ab′)₂ fragments).

Alternatively, antibodies of the present invention can be produced through the application of recombinant DNA and phage display technology or through synthetic chemistry using methods known in the art. For example, the antibodies of the present invention can be prepared using various phage display methods known in the art. In phage display methods, functional antibody domains are displayed on the surface of a phage particle which carries polynucleotide sequences encoding them. Phage with a desired binding property are selected from a repertoire or combinatorial antibody library (e.g. human or murine) by selecting directly with antigen, typically antigen bound or captured to a solid surface or bead. Phage used in these methods are typically filamentous phage including fd and M13 with Fab, Fv or disulfide stabilized Fv antibody domains recombinantly fused to either the phage gene III or gene VIII protein. Examples of phage display methods that can be used to make the antibodies of the present invention include those disclosed in Brinkman U. et al. (1995) J. Immunol. Methods 182:41-50; Ames, R. S. et al. (1995) J. Immunol. Methods 184:177-186; Kettleborough, C. A. et al. (1994) Eur. J. Immunol. 24:952-958; Persic, L. et al. (1997) Gene 187:9-18; Burton, D. R. et al. (1994) Advances in Immunology 57:191-280; PCT/GB91/01134; WO 90/02809; WO 91/10737; WO 92/01047; WO 92/18619; WO 93/11236; WO 95/15982; WO 95/20401; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743 (said references incorporated by reference in their entireties).

As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)₂ fragments can also be employed using methods known in the art such as those disclosed in WO 92/22324; Mullinax, R. L. et al. (1992) BioTechniques 12(6):864-869; and Sawai, H. et al. (1995) AJRI 34:26-34; and Better, M. et al. (1988) Science 240:1041-1043 (said references incorporated by reference in their entireties).

Further polypeptides of the invention relate to antibodies and T-cell antigen receptors (TCR) which immunospecifically bind a polypeptide, polypeptide fragment, or variant of SEQ ID NO:2 and/or 4, and/or an epitope, of the present invention (as determined by immunoassays well known in the art for assaying specific antibody-antigen binding). Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

Most preferably the antibodies are human antigen-binding antibody fragments of the present invention and include, but are not limited to, Fab, Fab′ and F(ab′)₂, Fd, single-chain Fvs (scFv), single-chain antibodies, disulfide-linked Fvs (sdfv) and fragments comprising either a VL or VH domain. Antigen-binding antibody fragments, including single-chain antibodies, may comprise the variable region(s) alone or in combination with the entirety or a portion of the following: hinge region, CH1, CH2, and CH3 domains. Also included in the invention are antigen-binding fragments also comprising any combination of variable region(s) with a hinge region, CH1, CH2, and CH3 domains. The antibodies of the invention may be from any animal origin including birds and mammals. Preferably, the antibodies are human, murine (e.g., mouse and rat), donkey, ship rabbit, goat, guinea pig, camel, horse, or chicken. As used herein, “human” antibodies include antibodies having the amino acid sequence of a human immunoglobulin and include antibodies isolated from human immunoglobulin libraries or from animals transgenic for one or more human immunoglobulin and that do not express endogenous immunoglobulins, as described infra and, for example in, U.S. Pat. No. 5,939,598 by Kucherlapati et al.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material. See, e.g., PCT publications WO 93/17715; WO 92/08802; WO 91/00360; WO 92/05793; Tutt, et al, J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992).

Antibodies of the present invention may be described or specified in terms of the epitope(s) or portion(s) of a polypeptide of the present invention which they recognize or specifically bind. The epitope(s) or polypeptide portion(s) may be specified as described herein, e.g., by N-terminal and C-terminal positions, by size in contiguous amino acid residues, or listed in the Tables and Figures. Preferred epitopes of the invention include: amino acids 2-14, 32-44, 47-60, 66-78, 87-103, 109-118, 146-162, 168-180, 183-219, 223-243, 275-284, 296-306, 314-334, 341-354, 357-376, 392-399, 401-410, 418-429, 438-454, 456-471, 474-488, 510-522, 524-538, 550-561, 565-626, 630-643, 659-671, 679-721, 734-749, 784-804, 813-820, 825-832, 845-854, 860-894, 899-917, 919-924 and 928-939 of SEQ ID NO:2 and amino acids 26-38, 45-52, 69-76, 80-99, 105-113, 129-136, 138-217, 254-263, 273-289, 294-313, 321-331, 339-356, 371-383, 417-427, 438-443, 459-471, 479-505, 507-526, 535-546, 550-607, 615-640, 648-653, 660-667, 669-681, 683-704, 717-732, 737-743, 775-787, 797-804, 811-825, 840-867 and 870-884 of SEQ ID NO:4, as well as polynucleotides that encode these epitopes. Antibodies which specifically bind any epitope or polypeptide of the present invention may also be excluded. Therefore, the present invention includes antibodies that specifically bind polypeptides of the present invention, and allows for the exclusion of the same.

Antibodies of the present invention may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention. In specific embodiments, antibodies of the present invention cross-react with murine, rat and/or rabbit homologs of human proteins and the corresponding epitopes thereof. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. In a specific embodiment, the above-described cross-reactivity is with respect to any single specific antigenic or immunogenic polypeptide, or combination(s) of 2,3, 4,5, or more of the specific antigenic and/or immunogenic polypeptides disclosed herein. Further included in the present invention are antibodies which bind polypeptides encoded by polynucleotides which hybridize to a polynucleotide of the present invention under stringent hybridization conditions (as described herein). Antibodies of the present invention may also be described or specified in terms of their binding affinity to a polypeptide of the invention. Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶M, 5×10⁻⁷M, 10⁷ M, 5×10⁻⁸ M, 10⁻⁸ M, 5×10⁻⁹M, 10⁻⁹ M, 5×10⁻¹⁰ M, 10⁻¹⁰M, 5×10⁻¹¹ M, 10⁻¹¹ M, 5×10⁻¹² M, 10⁻¹² M, 5×10⁻¹¹ M, 10⁻¹³ M, 5×10⁻¹⁴ M, 10⁻¹⁴ M, 5×10⁻¹⁵ M, or 10⁻¹⁵ M.

The invention also provides antibodies that competitively inhibit binding of an antibody to an epitope of the invention as determined by any method known in the art for determining competitive binding, for example, the immunoassays described herein. In preferred embodiments, the antibody competitively inhibits binding to the epitope by at least 95%, at least 90%, at least 85%, at least 80%, at least 75%, at least 70%, at least 60%, or at least 50%.

Antibodies of the present invention may act as agonists or antagonists of the polypeptides of the present invention. For example, the present invention includes antibodies which disrupt the receptor/ligand interactions with the polypeptides of the invention either partially or fully. Preferrably, antibodies of the present invention bind an antigenic epitope disclosed herein, or a portion thereof. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or its substrate by immunoprecipitation followed by western blot analysis (for example, as described supra). In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex, and, preferably, do not specifically recognize the unbound receptor or the unbound ligand. Likewise, included in the invention are neutralizing antibodies which bind the ligand and prevent binding of the ligand to the receptor, as well as, antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides of the invention disclosed herein. The above antibody agonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. 111(Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167(1998); Bartuneket al., Cytokine 8(1):14-20(1996) (which are all incorporated by reference herein in their entireties).

Antibodies of the present invention may be used, for example, but not limited to, to purify, detect, and target the polypeptides of the present invention, including both in vitro and in vivo diagnostic and therapeutic methods. For example, the antibodies have use in immunoassays for qualitatively and quantitatively measuring levels of the polypeptides of the present invention in biological samples. See, e.g., Harlow et al., Antibodies: A Laboratory Manual, (Cold Spring Harbor Laboratory Press, 2nd ed. 1988) (incorporated by reference herein in its entirety).

As discussed in more detail below, the antibodies of the present invention may be used either alone or in combination with other compositions. The antibodies may further be recombinantly fused to a heterologous polypeptide at the N- or C-terminus or chemically conjugated (including covalently and non-covalently conjugations) to polypeptides or other compositions. For example, antibodies of the present invention may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

The antibodies of the invention include derivatives that are modified, i.e, by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

The antibodies of the present invention may be generated by any suitable method known in the art. Polyclonal antibodies to an antigen-of-interest can be produced by various procedures well known in the art. For example, a polypeptide of the invention can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art and are discussed in detail in the Examples. In a non-limiting example, mice can be immunized with a polypeptide of the invention or a cell expressing such peptide. Once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with an antigen of the invention with myeloma cells and then screening the hybridomas resulting from the fusion for hybridoma clones that secrete an antibody able to bind a polypeptide of the invention.

Antibody fragments which recognize specific epitopes may be generated by known techniques. For example, Fab and F(ab′)₂ fragments of the invention may be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CH1 domain of the heavy chain.

For example, the antibodies of the present invention can also be generated using various phage display methods known in the art. In phage display methods, functional antibody domains are displayed on the surface of phage particles which carry the polynucleotide sequences encoding them. In a particular embodiment, such phage can be utilized to display antigen binding domains expressed from a repertoire or combinatorial antibody library (e.g., human or murine). Phage expressing an antigen binding domain that binds the antigen of interest can be selected or identified with antigen, e.g., using labeled antigen or antigen bound or captured to a solid surface or bead. Phage used in these methods are typically filamentous phage including fd and M13 binding domains expressed from phage with Fab, Fv or disulfide stabilized Fv antibody domains recombinantly fused to either the phage gene 1 ml or gene VIII protein. Examples of phage display methods that can be used to make the antibodies of the present invention include those disclosed in Brinkman et al., J. Immunol. Methods 182:41-50 (1995); Ames et al., J. Immunol. Methods 184:177-186 (1995); Kettleborough et al., Eur. J. Immunol. 24:952-958 (1994); Persic et al., Gene 187:9-18 (1997); Burton et al., Advances in Immunology 57:191-280 (1994); PCT application No. PCT/GB91/01134; PCT publications WO 90/02809; WO 91/10737; WO 92/01047; WO 92/18619; WO 93/11236; WO 95/15982; WO 95/20401; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described in detail below. For example, techniques to recombinantly produce Fab, Fab′ and F(ab′)₂ fragments can also be employed using methods known in the art such as those disclosed in PCT publication WO 92/22324; Mullinax et al., BioTechniques 12(6):864-869 (1992); and Sawai et al., AJRI 34:26-34 (1995); and Better et al., Science 240:1041-1043 (1988) (said references incorporated by reference in their entireties).

Examples of techniques which can be used to produce single-chain Fvs and antibodies include those described in U.S. Pat. Nos. 4,946,778 and 5,258,498; Huston et al., Methods in Enzymology 203:46-88 (1991); Shu et al., PNAS 90:7995-7999 (1993); and Skerra et al., Science 240:1038-1040 (1988). For some uses, including in vivo use of antibodies in humans and in vitro detection assays, it may be preferable to use chimeric, humanized, or human antibodies. A chimeric antibody is a molecule in which different portions of the antibody are derived from different animal species, such as antibodies having a variable region derived from a murine monoclonal antibody and a human immunoglobulin constant region. Methods for producing chimeric antibodies are 1known in the art. See e.g., Morrison, Science 229:1202 (1985); Oi et al., BioTechniques 4:214 (1986); Gillies et al., (1989) J. Immunol. Methods 125:191-202; U.S. Pat. Nos. 5,807,715; 4,816,567; and 4,816,397, which are incorporated herein by reference in their entirety. Humanized antibodies are antibody molecules from non-human species antibody that binds the desired antigen having one or more complementarity determining regions (CDRs) from the non-human species and a framework regions from a human immunoglobulin molecule. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; Riechmann et al., Nature 332:323 (1988), which are incorporated herein by reference in their entireties.) Antibodies can be humanized using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, Molecular Immunology 28(4/5):489-498 (1991); Studnicka et al., Protein Engineering 7(6):805-814 (1994); Roguska. et al., PNAS 91:969-973 (1994)), and chain shuffling (U.S. Pat. No. 5,565,332).

Completely human antibodies are particularly desirable for therapeutic treatment of human patients. Human antibodies can be made by a variety of methods known in the art including phage display methods described above using antibody libraries derived from human immunoglobulin sequences. See also, U.S. Pat. Nos. 4,444,887 and 4,716,111; and PCT publications WO 98/46645, WO 98/50433, WO 98/24893, WO 98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entirety. In addition, companies such as Abgenix, Inc. (Freemont, Calif.) and GenPharm (San Jose, Calif.) can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a mouse antibody, is used to guide the selection of a completely human antibody recognizing the same epitope. (Jespers et al., Bio/technology 12:899-903 (1988)).

Further, antibodies to the polypeptides of the invention can, in turn, be utilized to generate anti-idiotype antibodies that “mimic” polypeptides of the invention using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to and competitively inhibit polypeptide multimerization and/or binding of a polypeptide of the invention to a ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide of the invention and/or to bind its ligands/receptors, and thereby block its biological activity.

Examples of techniques which can be used to produce single-chain Fvs and antibodies include those described in U.S. Pat. Nos. 4,946,778 and 5,258,498; Huston et al. (1991) Methods in Enzymology 203:46-88; Shu, L. et al. (1993) PNAS 90:7995-7999; and Skerra, A. et al. (1988) Science 240:1038-1040. For some uses, including in vivo use of antibodies in humans and in vitro detection assays, it may be preferable to use chimeric, humanized, or human antibodies. Methods for producing chimeric antibodies are known in the art. See e.g., Morrison, Science 229:1202 (1985); Oi et al., BioTechniques 4:214 (1986); Gillies, S.D. et al. (1989) J. Immunol. Methods 125:191-202; and U.S. Pat. No. 5,807,715. Antibodies can be humanized using a variety of techniques including CDR-grafting (EP 0 239 400; WO 91/09967; U.S. Pat. Nos. 5,530,101; and 5,585,089), veneering or resurfacing (EP 0 592 106; EP 0 519 596; Padlan E. A., (1991) Molecular Immunology 28(4/5):489-498; Studnicka G. M. et al. (1994) Protein Engineering 7(6):805-814; Roguska M. A. et al. (1994) PNAS 91:969-973), and chain shuffling (U.S. Pat. No. 5,565,332). Human antibodies can be made by a variety of methods known in the art including phage display methods described above. See also, U.S. Pat. Nos. 4,444,887, 4,716,111, 5,545,806, and 5,814,318; and WO 98/46645 (said references incorporated by reference in their entireties).

Further included in the present invention are antibodies recombinantly fused or chemically conjugated (including both covalently and non-covalently conjugations) to a polypeptide of the present invention. The antibodies may be specific for antigens other than polypeptides of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in in vitro immunoassays and purification methods using methods known in the art. See e.g., Harbor et al. supra and WO 93/21232; EP 0 439 095; Naramura, M. et al. (1994) Immunol. Lett. 39:91-99; U.S. Pat. No. 5,474,981; Gillies, S. O. et al. (1992) PNAS 89:1428-1432; Fell, H. P. et al. (1991) J. Immunol. 146:2446-2452 (said references incorporated by reference in their entireties).

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the hinge region, CH1 domain, CH2 domain, and CH3 domain or any combination of whole domains or portions thereof. The polypeptides of the present invention may be fused or conjugated to the above antibody portions to increase the in vivo half life of the polypeptides or for use in immunoassays using methods known in the art. The polypeptides may also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See e.g., U.S. Pat. Nos. 5,336,603, 5,622,929, 5,359,046, 5,349,053, 5,447,851, 5,112,946; EP 0 307 434, EP 0 367 166; WO 96/04388, WO 91/06570; Ashkenazi, A. et al. (1991) PNAS 88:10535-10539; Zheng, X. X. et al. (1995) J. Immunol. 154:5590-5600; and Vil, H. et al. (1992) PNAS 89:11337-11341 (said references incorporated by reference in their entireties).

The invention further relates to antibodies that act as agonists or antagonists of the polypeptides of the present invention. Antibodies which act as agonists or antagonists of the polypeptides of the present invention include, for example, antibodies which disrupt receptor/ligand interactions with the polypeptides of the invention either partially or fully. For example, the present invention includes antibodies that disrupt the ability of the proteins of the invention to multimerize. In another example, the present invention includes antibodies which allow the proteins of the invention to multimerize, but disrupt the ability of the proteins of the invention to bind one or more METH1 and/or METH2 receptor(s)/ligand(s). In yet another example, the present invention includes antibodies which allow the proteins of the invention to multimerize, and bind METH1 and/or METH2 receptor(s)/ligand(s), but blocks biological activity associated with the METH1 and/or METH2/receptor/ligand complex.

Antibodies which act as agonists or antagonists of the polypeptides of the present invention also include, both receptor-specific antibodies and ligand-specific antibodies. Included are receptor-specific antibodies that do not prevent ligand binding but prevent receptor activation. Receptor activation (i.e., signaling) may be determined by techniques described herein or otherwise known in the art. Also included are receptor-specific antibodies which both prevent ligand binding and receptor activation. Likewise, included are neutralizing antibodies which bind the ligand and prevent binding of the ligand to the receptor, as well as antibodies which bind the ligand, thereby preventing receptor activation, but do not prevent the ligand from binding the receptor. Further included are antibodies that activate the receptor. These antibodies may act as agonists for either all or less than all of the biological activities affected by ligand-mediated receptor activation. The antibodies may be specified as agonists or antagonists for biological activities comprising specific activities disclosed herein. The above antibody agonists can be made using methods known in the art. See e.g., WO 96/40281; U.S. Pat. No. 5,811,097; Deng, B. et al., Blood 92(6):1981-1988 (1998); Chen, Z. et al., Cancer Res. 58(16):3668-3678 (1998); Harrop, J. A. et al., J. Immunol. 161(4):1786-1794 (1998); Zhu, Z. et al., Cancer Res. 58(15):3209-3214 (1998); Yoon, D. Y. et al., J. Immunol. 160(7):3170-3179 (1998); Prat, M. et al., J. Cell. Sci. 111(Pt2):237-247 (1998); Pitard, V. et al., J. Immunol. Methods 205(2):177-190 (1997); Liautard, J. et al., Cytokine 9(4):233-241 (1997); Carlson, N. G. et al., J. Biol. Chem. 272(17):11295-11301(1997); Taryman, R. E. et al., Neuron 14(4):755-762 (1995); Muller, Y. A. et al., Structure 6(9):1153-1167 (1998); Bartunek, P. et al., Cytokine 8(1): 14-20 (1996) (said references incorporated by reference in their entireties).

As discussed above, antibodies to the METH1 and/or METH2 proteins of the invention can, in turn, be utilized to generate anti-idiotype antibodies that “mimic” METH1 and/or METH2 using techniques well known to those skilled in the art. (See, e.g., Greenspan & Bona, FASEB J. 7(5):437-444; (1989) and Nissinoff, J. Immunol. 147(8):2429-2438 (1991)). For example, antibodies which bind to METH1 and/or METH2 and competitively inhibit METH1 and/or METH2 multimerization and/or binding to ligand can be used to generate anti-idiotypes that “mimic” the METH1 and/or METH2 mutimerization and/or binding domain and, as a consequence, bind to and neutralize METH1 and/or METH2 and/or its ligand. Such neutralizing anti-idiotypes or Fab fragments of such anti-idiotypes can be used in therapeutic regimens to neutralize METH1 and/or METH2 ligand. For example, such anti-idiotypic antibodies can be used to bind METH1 and/or METH2, or to bind METH1 and/or METH2 ligands/receptors, and thereby block METH1 and/or METH2 biological activity.

Polynucleotides Encoding Antibodies

The invention further provides polynucleotides comprising a nucleotide sequence encoding an antibody of the invention and fragments thereof. The invention also encompasses polynucleotides that hybridize under stringent or lower stringency hybridization conditions, e.g., as defined supra, to polynucleotides that encode an antibody, preferably, that specifically binds to a polypeptide of the invention, preferably, an antibody that binds to a polypeptide having the amino acid sequence of SEQ ID NO:2 and/or 4.

The polynucleotides may be obtained, and the nucleotide sequence of the polynucleotides determined, by any method known in the art. For example, if the nucleotide sequence of the antibody is known, a polynucleotide encoding the antibody may be assembled from chemically synthesized oligonucleotides (e.g., as described in Kutmeier et al., BioTechniques 1 7:242 (1994)), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence and corresponding amino acid sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties ), to generate antibodies having a different amino acid sequence, for example to create amino acid substitutions, deletions, and/or insertions.

In a specific embodiment, the amino acid sequence of the heavy and/or light chain variable domains may be inspected to identify the sequences of the complementarity determining regions (CDRs) by methods that are well know in the art, e.g., by comparison to known amino acid sequences of other heavy and light chain variable regions to determine the regions of sequence hypervariability. Using routine recombinant DNA techniques, one or more of the CDRs may be inserted within framework regions, e.g., into human framework regions to humanize a non-human antibody, as described supra. The framework regions may be naturally occurring or consensus framework regions, and preferably human framework regions (see, e.g., Chothia et al., J. Mol. Biol. 278:457-479 (1998) for a listing of human framework regions). Preferably, the polynucleotide generated by the combination of the framework regions and CDRs encodes an antibody that specifically binds a polypeptide of the invention. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

In addition, techniques developed for the production of “chimeric antibodies” (Morrison et al., Proc. Natl. Acad. Sci. 81:851-855 (1984); Neuberger et al., Nature 312:604-608 (1984); Takeda et al., Nature 314:452-454 (1985)) by splicing genes from a mouse antibody molecule of appropriate antigen specificity together with genes from a human antibody molecule of appropriate biological activity can be used. As described supra, a chimeric antibody is a molecule in which different portions are derived from different animal species, such as those having a variable region derived from a murine mAb and a human immunoglobulin constant region, e.g., humanized antibodies.

Alternatively, techniques described for the production of single chain antibodies (U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989)) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli may also be used (Skerra et al., Science 242:1038-1041 (1988)).

Methods of Producing Antibodies

The antibodies of the invention can be produced by any method known in the art for the synthesis of antibodies, in particular, by chemical synthesis or preferably; by recombinant expression techniques.

Recombinant expression of an antibody of the invention, or fragment, derivative or analog thereof, (e.g., a heavy or light chain of an antibody of the invention or a single chain antibody of the invention), requires construction of an expression vector containing a polynucleotide that encodes the antibody. Once a polynucleotide encoding an antibody molecule or a heavy or light chain of an antibody, or portion thereof (preferably containing the heavy or light chain variable domain), of the invention has been obtained, the vector for the production of the antibody molecule may be produced by recombinant DNA technology using techniques well known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, or a heavy or light chain thereof, or a heavy or light chain variable domain, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy or light chain.

The expression vector is transferred to a host cell by conventional techniques and the transfected cells are then cultured by conventional techniques to produce an antibody of the invention. Thus, the invention includes host cells containing a polynucleotide encoding an antibody of the invention, or a heavy or light chain thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention. Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., Gene 45:101 (1986); Cockett et al., Bio/Technology 8:2 (1990)).

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited, to the E. coli expression vector pUR278 (Ruther et al., EMBO J. 2:1791 (1983)), in which the antibody coding sequence may be ligated individually into the vector in frame with the lac Z coding region so that a fusion protein is produced; pIN vectors (Inouye & Inouye, Nucleic Acids Res. 13:3101-3109 (1985); Van Heeke & Schuster, J. Biol. Chem. 24:5503-5509 (1989)); and the like. pGEX vectors may also be used to express foreign polypeptides as fusion proteins with glutathione S-transferase (GST). In general, such fusion proteins are soluble and can easily be purified from lysed cells by adsorption and binding to matrix glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts. (e.g., see Logan & Shenk, Proc. Natl. Acad. Sci. USA 81:355-359 (1984)). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., Methods in Enzymol. 153:51-544 (1987)).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERA, BHK, Hela, COS, MDCK, 293, 3T3, WI38, and in particular, breast cancer cell lines such as, for example, BT483, Hs578T, HTB2, BT20 and T47D, and normal mammary gland cell line such as, for example, CRL7030 and Hs578Bst.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compounds that interact directly or indirectly with the antibody molecule.

A number of selection systems may be used, including but not limited to the herpes simplex virus thymidinekinase (Wigler et al., Cell 11:223 (1977)), hypoxanthineguanine phosphoribosyltransferase (Szybalska & Szybalski, Proc. Natl. Acad. Sci. USA 48:202 (1992)), and adenine phosphoribosyltransferase (Lowy et al., Cell 22:817 (1980)) genes can be employed in tk-, hgprt- or aprt-cells, respectively. Also, antimetabolite resistance can be used as the basis of selection for the following genes: dhfr, which confers resistance to methotrexate (Wigler et al., Natl. Acad. Sci. USA 77:357 (1980); O'Hare et al., Proc. Natl. Acad. Sci. USA 78:1527 (1981)); gpt, which confers resistance to mycophenolic acid (Mulligan & Berg, Proc. Natl. Acad. Sci. USA 78:2072 (1981)); neo, which confers resistance to the aminoglycoside G-418 (Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, 1993, TIB TECH 11(5): 155-215); and hygro, which confers resistance to hygromycin (Santerre et al., Gene 30:147 (1984)). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., J. Mol. Biol. 150:1 (1981), which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., Mol. Cell. Biol. 3:257 (1983)).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, Nature 322:52 (1986); Kohler, Proc. Natl. Acad. Sci. USA 77:2197 (1980)). The coding sequences for the heavy and light chains may comprise cDNA or genomic DNA.

Once an antibody molecule of the invention has been produced by an animal, chemically synthesized, or recombinantly expressed, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. In addition, the antibodies of the present invention or fragments thereof can be fused to heterologous polypeptide sequences described herein or otherwise known in the art, to facilitate purification.

The present invention encompasses antibodies recombinantly fused or chemically conjugated (including both covalently and non-covalently conjugations) to a polypeptide (or portion thereof, preferably at least 10, 20, 30, 40, 50, 60, 70, 80, 90 or 100 amino acids of the polypeptide) of the present invention to generate fusion proteins. The fusion does not necessarily need to be direct, but may occur through linker sequences. The antibodies may be specific for antigens other than polypeptides (or portion thereof, preferably at least 10, 20, 30, 40, 50, 60, 70, 80, 90 or 100 amino acids of the polypeptide) of the present invention. For example, antibodies may be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention may also be used in in vitro immunoassays and purification methods using methods known in the art. See e.g., Harbor et al., supra, and PCT publication WO 93/21232; EP 439,095; Naramura et al., Immunol. Lett. 39:91-99 (1994); U.S. Pat. No. 5,474,981; Gillies et al., PNAS 89:1428-1432 (1992); Fell et al., J. Immunol. 146:2446-2452(1991), which are incorporated by reference in their entireties.

The present invention further includes compositions comprising the polypeptides of the present invention fused or conjugated to antibody domains other than the variable regions. For example, the polypeptides of the present invention may be fused or conjugated to an antibody Fc region, or portion thereof. The antibody portion fused to a polypeptide of the present invention may comprise the constant region, hinge region, CH1 domain, CH2 domain, and CH3 domain or any combination of whole domains or portions thereof. The polypeptides may also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See, e.g., U.S. Pat. Nos. 5,336,603; 5,622,929; 5,359,046; 5,349,053; 5,447,851; 5,112,946; EP 307,434; EP 367,166; PCT publications WO 96/04388; WO 91/06570; Ashkenazi et al., Proc. Natl. Acad. Sci. USA 88:10535-10539 (1991); Zheng et al., J. Immunol. 154:5590-5600 (1995); and Vil et al., Proc. Natl. Acad. Sci. USA 89:11337-11341(1992) (said references incorporated by reference in their entireties).

As discussed, supra, the polypeptides corresponding to a polypeptide, polypeptide fragment, or a variant of SEQ ID NO:2 and/or 4 may be fused or conjugated to the above antibody portions to increase the in vivo half life of the polypeptides or for use in immunoassays using methods known in the art. Further, the polypeptides corresponding to SEQ ID NO:2 and/or 4 may be fused or conjugated to the above antibody portions to facilitate purification. One reported example describes chimeric proteins consisting of the first two domains of the human CD4-polypeptide and various domains of the constant regions of the heavy or light chains of mammalian immunoglobulins. (EP 394,827; Traunecker et al., Nature 331:84-86 (1988). The polypeptides of the present invention fused or conjugated to an antibody having disulfide-linked dimeric structures (due to the IgG) may also be more efficient in binding and neutralizing other molecules, than the monomeric secreted protein or protein fragment alone. (Fountoulakis et al., J. Biochem. 270:3958-3964 (1995)). In many cases, the Fc part in a fusion protein is beneficial in therapy and diagnosis, and thus can result in, for example, improved pharmacokinetic properties. (EP A 232,262). Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5 receptor, have been fused with Fc portions for the purpose of high-throughput screening assays to identify antagonists of hIL-5. (See, Bennett et al., J. Molecular Recognition 8:52-58 (1995); Johanson et al., J. Biol. Chem. 270:9459-9471 (1995).)

Moreover, the antibodies or fragments thereof of the present invention can be fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

The present invention further encompasses antibodies or fragments thereof conjugated to a diagnostic or therapeutic agent. The antibodies can be used diagnostically to, for example, monitor the development or progression of a tumor as part of a clinical testing procedure to, e.g., determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody (or fragment thereof) or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include 125I, ¹³¹I, ¹¹¹In or ⁹⁹Tc

Further, an antibody or fragment thereof may be conjugated to a therapeutic moiety such as a cytotoxin, e.g., a cytostatic or cytocidal agent, a therapeutic agent or a radioactive metal ion, e.g., alpha-emitters such as, for example, 213Bi. A cytotoxin or cytotoxic agent includes any agent that is detrimental to cells. Examples include paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (1H) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response, the therapeutic agent or drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, α-interferon, β-interferon, nerve growth factor, platelet derived growth factor, tissue plasminogen activator, an apoptotic agent, e.g., TNF-α, TNF-β, AIM I (See, International Publication No. WO 97/33899), AIM II (See, International Publication No. WO 97/34911), Fas Ligand (Takahashi et al., Int. Immunol., 6:1567-1574 (1994)), VEGI (See, International Publication No. WO 99/23105), a thrombotic agent or an anti-angiogenic agent, e.g., angiostatin or endostatin; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Techniques for conjugating such therapeutic moiety to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp.243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev. 62:119-58 (1982).

Alternatively, an antibody can be conjugated to a second antibody to form an antibody heteroconjugate as described by Segal in U.S. Pat. No. 4,676,980, which is incorporated herein by reference in its entirety.

An antibody, with or without a therapeutic moiety conjugated to it, administered alone or in combination with cytotoxic factor(s) and/or cytokine(s) can be used as a therapeutic.

Immunophenotyping

The antibodies of the invention may be utilized for immunophenotyping of cell lines and biological samples. The translation product of the gene of the present invention may be useful as a cell specific marker, or more specifically as a cellular marker that is differentially expressed at various stages of differentiation and/or maturation of particular cell types. Monoclonal antibodies directed against a specific epitope, or combination of epitopes, will allow for the screening of cellular populations expressing the marker. Various techniques can be utilized using monoclonal antibodies to screen for cellular populations expressing the marker(s), and include magnetic separation using antibody-coated magnetic beads, “panning” with antibody attached to a solid matrix (i.e., plate), and flow cytometry. (See, e.g., U.S. Pat. No. 5,985,660; and Morrison et al., Cell, 96:737-49 (1999)).

These techniques allow for the screening of particular populations of cells, such as might be found with hematological malignancies (i.e. minimal residual disease (MRD) in acute leukemic patients) and “non-self” cells in transplantations to prevent Graft-versus-Host Disease (GVHD). Alternatively, these techniques allow for the screening of hematopoietic stem and progenitor cells capable of undergoing proliferation and/or differentiation, as might be found in human umbilical cord blood.

Assays For Antibody Binding

The antibodies of the invention may be assayed for immunospecific binding by any method known in the art. The immunoassays which can be used include but are not limited to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 14 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds, 1994, Current Protocols in Molecular Biology, Vol. 1, John Wiley & Sons, Inc., New York at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound (e.g., ³H or ¹²⁵I) in the presence of increasing amounts of an unlabeled second antibody.

Therapeutic Uses Of Antibodies of the Invention

The present invention is further directed to antibody-based therapies which involve administering antibodies of the invention to an animal, preferably a mammal, and most preferably a human, patient for treating one or more of the disclosed diseases, disorders, or conditions. Therapeutic compounds of the invention include, but are not limited to, antibodies of the invention (including fragments, analogs and derivatives thereof as described herein) and nucleic acids encoding antibodies of the invention (including fragments, analogs and derivatives thereof and anti-idiotypic antibodies as described herein). The antibodies of the invention can be used to treat, inhibit or prevent diseases, disorders or conditions associated with aberrant expression and/or activity of a polypeptide of the invention, including, but not limited to, any one or more of the diseases, disorders, or conditions described herein. The treatment and/or prevention of diseases, disorders, or conditions associated with aberrant expression and/or activity of a polypeptide of the invention includes, but is not limited to, alleviating symptoms associated with those diseases, disorders or conditions. Antibodies of the invention may be provided in pharmaceutically acceptable compositions as known in the art or as described herein.

A summary of the ways in which the antibodies of the present invention may be used therapeutically includes binding polynucleotides or polypeptides of the present invention locally or systemically in the body or by direct cytotoxicity of the antibody, e.g. as mediated by complement (CDC) or by effector cells (ADCC). Some of these approaches are described in more detail below. Armed with the teachings provided herein, one of ordinary skill in the art will know how to use the antibodies of the present invention for diagnostic, monitoring or therapeutic purposes without undue experimentation.

The antibodies of this invention may be advantageously utilized in combination with other monoclonal or chimeric antibodies, or with lymphokines or hematopoietic growth factors (such as, e.g., IL-2, IL-3 and IL-7), for example, which serve to increase the number or activity of effector cells which interact with the antibodies.

The antibodies of the invention may be administered alone or in combination with other types of treatments (e.g., radiation therapy, chemotherapy, hormonal therapy, immunotherapy and anti-tumor agents). Generally, administration of products of a species origin or species reactivity (in the case of antibodies) that is the same species as that of the patient is preferred. Thus, in a preferred embodiment, human antibodies, fragments derivatives, analogs, or nucleic acids, are administered to a human patient for therapy or prophylaxis.

It is preferred to use high affinity and/or potent in vivo inhibiting and/or neutralizing antibodies against polypeptides or polynucleotides of the present invention, fragments or regions thereof, for both immunoassays directed to and therapy of disorders related to polynucleotides or polypeptides, including fragments thereof, of the present invention. Such antibodies, fragments, or regions, will preferably have an affinity for polynucleotides or polypeptides of the invention, including fragments thereof. Preferred binding affinities include those with a dissociation constant or Kd less than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, and 10⁻¹⁵ M.

Diagnosis and Imaging

Labeled antibodies, and derivatives and analogs thereof, which specifically bind to a polypeptide of interest can be used for diagnostic purposes to detect, diagnose, or monitor diseases, disorders, and/or conditions associated with the aberrant expression and/or activity of a polypeptide of the invention. The invention provides for the detection of aberrant expression of a polypeptide of interest, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of aberrant expression.

The invention provides a diagnostic assay for diagnosing a disorder, comprising (a) assaying the expression of the polypeptide of interest in cells or body fluid of an individual using one or more antibodies specific to the polypeptide interest and (b) comparing the level of gene expression with a standard gene expression level, whereby an increase or decrease in the assayed polypeptide gene expression level compared to the standard expression level is indicative of a particular disorder. With respect to cancer, the presence of a relatively high amount of transcript in biopsied tissue from an individual may indicate a predisposition for the development of the disease, or may provide a means for detecting the disease prior to the appearance of actual clinical symptoms. A more definitive diagnosis of this type may allow health professionals to employ preventative measures or aggressive treatment earlier thereby preventing the development or further progression of the cancer.

Antibodies of the invention can be used to assay protein levels in a biological sample using classical immunohistological methods known to those of skill in the art (e.g., see Jalkanen, et al., J. Cell. Biol. 101:976-985 (1985); Jalkanen, et al., J. Cell. Biol. 105:3087-3096(1987)). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (3H), indium (112In), and technetium (⁹⁹Tc); luminescent labels, such as luminol; and fluorescent labels, such as fluorescein and rhodamine, and biotin.

One aspect of the invention is the detection and diagnosis of a disease or disorder associated with aberrant expression of a polypeptide of interest in an animal, preferably a mammal and most preferably a human. In one embodiment, diagnosis comprises: a) administering (for example, parenterally, subcutaneously, or intraperitoneally) to a subject an effective amount of a labeled molecule which specifically binds to the polypeptide of interest; b) waiting for a time interval following the administering for permitting the labeled molecule to preferentially concentrate at sites in the subject where the polypeptide is expressed (and for unbound labeled molecule to be cleared to background level); c) determining background level; and d) detecting the labeled molecule in the subject, such that detection of labeled molecule above the background level indicates that the subject has a particular disease or disorder associated with aberrant expression of the polypeptide of interest. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹mTc. The labeled antibody or antibody fragment will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

In an embodiment, monitoring of the disease or disorder is carried out by repeating the method for diagnosing the disease or disease, for example, one month after initial diagnosis, six months after initial diagnosis, one year after initial diagnosis, etc.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patent using positron emission-tomography. In yet another embodiment, the molecule is labeled with a paramagnetic label and is detected in a patient using magnetic resonance imaging (MRI).

Fusion Proteins

Any METH1 or METH2 polypeptide can be used to generate fusion proteins. For example, the METH1 or METH2 polypeptide, when fused to a second protein, can be used as an antigenic tag. Antibodies raised against the METH1 or METH2 polypeptide can be used to indirectly detect the second protein by binding to the METH1 or METH2. Moreover, because secreted proteins target cellular locations based on trafficking signals, the METH1 or METH2 polypeptides can be used as a targeting molecule once fused to other proteins.

Examples of domains that can be fused to METH1 or METH2 polypeptides include not only heterologous signal sequences, but also other heterologous functional regions. The fusion does not necessarily need to be direct, but may occur through linker sequences.

In certain preferred embodiments, METH1 or METH2 proteins of the invention comprise fusion proteins wherein the METH1 or METH2 polypeptides are those described above as m₁-n₁ or m₂-n₂, respectively. In preferred embodiments, the application is directed to nucleic acid molecules at least 80%, 85%, 90%, 91%, 92%, 93%, 94%, 95%, 96%, 97%, 98% or 99% identical to the nucleic acid sequences encoding polypeptides having the amino acid sequence of the specific - and C-terminal deletions recited herein. Polynucleotides encoding these polypeptides are also encompassed by the invention.

Moreover, fusion proteins may also be engineered to improve characteristics of the METH1 or METH2 polypeptide. For instance, a region of additional amino acids, particularly charged amino acids, may be added to the N-terminus of the METH1 or METH2 polypeptide to improve stability and persistence during purification from the host cell or subsequent handling and storage. Also, peptide moieties may be added to the METH1 or METH2 polypeptide to facilitate purification. Such regions may be removed prior to final preparation of the METH1 or METH2 polypeptide. The addition of peptide moieties to facilitate handling of polypeptides are familiar and routine techniques in the art.

Moreover, METH1 or METH2 polypeptides, including fragments, and specifically epitopes, can be combined with parts of the constant domain of immunoglobulins (IgG), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. (EP A 394,827; Traunecker et al., Nature 331:84-86 (1988).) Fusion proteins having disulfide-linked dimeric structures (due to the IgG) can also be more efficient in binding and neutralizing other molecules, than the monomeric secreted protein or protein fragment alone. (Fountoulakis et al., J. Biochem. 270:3958-3964 (1995).)

Similarly, EP-A-O 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, for example, improved pharmacokinetic properties. (EP-A 0 232 262.) Alternatively, deleting the Fc part after the fusion protein has been expressed, detected, and purified, would be desired. For example, the Fc portion may hinder therapy and diagnosis if the fusion protein is used as an antigen for immunizations. In drug discovery, for example, human proteins, such as hIL-5 receptor, have been fused with Fc portions for the purpose of high-throughput screening assays to identify antagonists of hIL-5. (See, D. Bennett et al., J. Molecular Recognition 8:52-58 (1995); K. Johanson et al., J. Biol. Chem. 270:9459-9471 (1995).)

Moreover, the METH1 or METH2 polypeptides can be fused to marker sequences, such as a peptide which facilitates purification of METH1 or METH2. In preferred embodiments, the marker amino acid sequence is a hexa-histidine peptide, such as the tag provided in a pQE vector (QIAGEN, Inc., 9259 Eton Avenue, Chatsworth, Calif., 9131 1), among others, many of which are commercially available. As described in Gentz et al., Proc. Natl. Acad. Sci. USA 86:821-824 (1989), for instance, hexa-histidine provides for convenient purification of the fusion protein. Another peptide tag useful for purification, the “HA” tag, corresponds to an epitope derived from the influenza hemagglutinin protein. (Wilson et al., Cell 37:767 (1984).)

Thus, any of these above fusions can be engineered using the METH1 or METH2 polynucleotides or the polypeptides.

Biological Activities of METH1 and/or METH2

METH1 and/or METH2 polynucleotides or polypeptides, or agonists or antagonists of METH1 and/or METH2, can be used in assays to test for one or more biological activities. If METH1 and/or METH2 polynucleotides or polypeptides, or agonists or antagonists of METH1 and/or METH2, do exhibit activity in a particular assay, it is likely that METH1 and/or METH2 may be involved in the diseases associated with the biological activity. Therefore, METH1 and/or METH2 could be used to treat the associated disease.

Immune Activity

METH1 and/or METH2 polynucleotides or polypeptides, or agonists or antagonists of METH1 and/or METH2, may be useful in treating deficiencies or disorders of the immune system, by activating or inhibiting the proliferation, differentiation, or mobilization (chemotaxis) of immune cells. Immune cells develop through a process called hematopoiesis, producing myeloid (platelets, red blood cells, neutrophils, and macrophages) and lymphoid (B and T lymphocytes) cells from pluripotent stem cells. The etiology of these immune deficiencies or disorders may be genetic, somatic, such as cancer or some autoimmune disorders, acquired (e.g., by chemotherapy or toxins), or infectious. Moreover, METH1 and/or METH2 polynucleotides or polypeptides, or agonists or antagonists of METH1 and/or METH2, can be used as a marker or detector of a particular immune system disease or disorder.

METH1 and/or METH2 polynucleotides or polypeptides, or agonists or antagonists of METH1 and/or METH2, may be useful in treating or detecting deficiencies or disorders of hematopoietic cells. METH1 and/or METH2 polynucleotides or polypeptides, or agonists or antagonists of METH1 and/or METH2, could be used to increase differentiation and proliferation of hematopoietic cells, including the pluripotent stem cells, in an effort to treat those disorders associated with a decrease in certain (or many) types hematopoietic cells. Examples of immunologic deficiency syndromes include, but are not limited to: blood protein disorders (e.g. agammaglobulinemia, dysgammaglobulinemia), ataxia telangiectasia, common variable immunodeficiency, Digeorge Syndrome, HIV infection, HTLV-BLV infection, leukocyte adhesion deficiency syndrome, lymphopenia, phagocyte bactericidal dysfunction, severe combined immunodeficiency (SCIDs), Wiskott-Aldrich Disorder, anemia, thrombocytopenia, or hemoglobinuria.

Moreover, METH1 and/or METH2 polynucleotides or polypeptides, or agonists or antagonists of METH1 and/or METH2, can also be used to modulate hemostatic (the stopping of bleeding) or thrombolytic activity (clot formation). For example, by increasing hemostatic or thrombolytic activity, METH1 and/or METH2 polynucleotides or polypeptides, or agonists or antagonists of METH1 and/or METH2, could be used to treat blood coagulation disorders (e.g., afibrinogenemia, factor deficiencies), blood platelet disorders (e.g. thrombocytopenia), or wounds resulting from trauma, surgery, or other causes. Alternatively, METH1 and/or METH2 polynucleotides or polypeptides, or agonists or antagonists of METH1 and/or METH2, that can decrease hemostatic or thrombolytic activity could be used to inhibit or dissolve clotting, important in the treatment of heart attacks (infarction), strokes, or scarring.

METH1 and/or METH2 polynucleotides or polypeptides, or agonists or antagonists of METH1 and/or METH2, may also be useful in treating or detecting autoimmune disorders. Many autoimmune disorders result from inappropriate recognition of self as foreign material by immune cells. This inappropriate recognition results in an immune response leading to the destruction of the host tissue. Therefore, the administration of METH1 and/or METH2 polynucleotides or polypeptides, or agonists or antagonists of METH1 and/or METH2, that can inhibit an immune response, particularly the proliferation, differentiation, or chemotaxis of T-cells, may be an effective therapy in preventing autoimmune disorders.

Examples of autoimmune disorders that can be treated or detected include, but are not limited to: Addison's Disease, hemolytic anemia, antiphospholipid syndrome, rheumatoid arthritis, dermatitis, allergic encephalomyelitis, glomerulonephritis, Goodpasture's Syndrome, Graves' Disease, Multiple Sclerosis, Myasthenia Gravis, Neuritis, Ophthalmia, Bullous Pemphigoid, Pemphigus, Polyendocrinopathies, Purpura, Reiter's Disease, Stiff-Man Syndrome, Autoimmune Thyroiditis, Systemic Lupus Erythematosus, Autoimmune Pulmonary Inflammation, Guillain-Barre Syndrome, insulin dependent diabetes mellitis, and autoimmune inflammatory eye disease.

Similarly, allergic reactions and conditions, such as asthma (particularly allergic asthma) or other respiratory problems, may also be treated by METH1 and/or METH2 polynucleotides or polypeptides, or agonists or antagonists of METH1 and/or METH2. Moreover, these molecules can be used to treat anaphylaxis, hypersensitivity to an antigenic molecule, or blood group incompatibility.

METH1 and/or METH2 polynucleotides or polypeptides, or agonists or antagonists of METH1 and/or METH2, may also be used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of METH1 and/or METH2 polynucleotides or polypeptides, or agonists or antagonists of METH1 and/or METH2, that inhibits an immune response, particularly the proliferation, differentiation, or chemotaxis of T-cells, may be an effective therapy in preventing organ rejection or GVHD.

Similarly, METH1 and/or METH2 polynucleotides or polypeptides, or agonists or antagonists of METH1 and/or METH2, may also be used to modulate inflammation. For example, METH1 and/or METH2 polynucleotides or polypeptides, or agonists or antagonists of METH1 and/or METH2, may inhibit the proliferation and differentiation of cells involved in an inflammatory response. These molecules can be used to treat inflammatory conditions, both chronic and acute conditions, including inflammation associated with infection (e.g., septic shock, sepsis, or systemic inflammatory response syndrome (SIRS)), ischemia-reperfusion injury, endotoxin lethality, arthritis, complement-mediated hyperacute rejection, nephritis, cytokine or chemokine induced lung injury, inflammatory bowel disease, Crohn's disease, or resulting from over production of cytokines (e.g., TNF or IL-1.)

Hyperproliferative Disorders

METH1 and/or METH12 polynucleotides or polypeptides, or agonists or antagonists of METH1 and/or METH2, can be used to treat or detect hyperproliferative disorders, including neoplasms. METH1 and/or METH2 polynucleotides or polypeptides, or agonists or antagonists of METH1 and/or METH2, may inhibit the proliferation of the disorder through direct or indirect interactions. Alternatively, METH1 and/or METH2 polynucleotides or polypeptides, or agonists or antagonists of METH1 and/or METH2, may proliferate other cells which can inhibit the hyperproliferative disorder.

For example, by increasing an immune response, particularly increasing antigenic qualities of the hyperproliferative disorder or by proliferating, differentiating, or mobilizing T-cells, hyperproliferative disorders can be treated. This immune response may be increased by either enhancing an existing immune response, or by initiating a new immune response. Alternatively, decreasing an immune response may also be a method of treating hyperproliferative disorders, such as a chemotherapeutic agent.

Examples of hyperproliferative disorders that can be treated or detected by METH1 and/or METH2 polynucleotides or polypeptides, or agonists or antagonists of METH1 and/or METH2, include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative disorders can also be treated or detected by METH1 and/or METH2 polynucleotides or polypeptides, or agonists or antagonists of METH1 and/or METH2. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

Cardiovascular Disorders

METH1 and/or METH2 polynucleotides or polypeptides, or agonists or antagonists of METH1 and/or METH2, encoding METH1 and/or METH2 may be used to treat cardiovascular disorders, including peripheral artery disease, such as limb ischemia.

Cardiovascular disorders include cardiovascular abnormalities, such as arterio-arterial fistula, arteriovenous fistula, cerebral arteriovenous malformations, congenital heart defects, pulmonary atresia, and Scimitar Syndrome. Congenital heart defects include aortic coarctation, cor triatriatum, coronary vessel anomalies, crisscross heart, dextrocardia, patent ductus arteriosus, Ebstein's anomaly, Eisenmenger complex, hypoplastic left heart syndrome, levocardia, tetralogy of fallot, transposition of great vessels, double outlet right ventricle, tricuspid atresia, persistent truncus arteriosus, and heart septal defects, such as aortopulmonary septal defect, endocardial cushion defects, Lutembacher's Syndrome, trilogy of Fallot, ventricular heart septal defects.

Cardiovascular disorders also include heart disease, such as arrhythmias, carcinoid heart disease, high cardiac output, low cardiac output, cardiac tamponade, endocarditis (including bacterial), heart aneurysm, cardiac arrest, congestive heart failure, congestive cardiomyopathy, paroxysmal dyspnea, cardiac edema, heart hypertrophy, congestive cardiomyopathy, left ventricular hypertrophy, right ventricular hypertrophy, post-infarction heart rupture, ventricular septal rupture, heart valve diseases, myocardial diseases, myocardial ischemia, pericardial effusion, pericarditis (including constrictive and tuberculous), pneumopericardium, postpericardiotomy syndrome, pulmonary heart disease, rheumatic heart disease, ventricular dysfunction, hyperemia, cardiovascular pregnancy complications, Scimitar Syndrome, cardiovascular syphilis, and cardiovascular tuberculosis.

Arrhythmias include sinus arrhythmia, atrial fibrillation, atrial flutter, bradycardia, extrasystole, Adams-Stokes Syndrome, bundle-branch block, sinoatrial block, long QT syndrome, parasystole, Lown-Ganong-levine Syndrome, Mahaim-type pre-excitation syndrome, Wolff-Parkinson-White syndrome, sick sinus syndrome, tachycardias, and ventricular fibrillation. Tachycardias include paroxysmal tachycardia, supraventricular tachycardia, accelerated idioventricular rhythm, atrioventricular nodal reentry tachycardia, ectopic atrial tachycardia, ectopic junctional tachycardia, sinoatrial nodal reentry tachycardia, sinus tachycardia, Torsades de Pointes, and ventricular tachycardia.

Heart valve diseases include aortic valve insufficiency, aortic valve stenosis, hear murmurs, aortic valve prolapse, mitral valve prolapse, tricuspid valve prolapse, mitral valve insufficiency, mitral valve stenosis, pulmonary atresia, pulmonary valve insufficiency, pulmonary valve stenosis, tricuspid atresia, tricuspid valve insufficiency, and tricuspid valve stenosis.

Myocardial diseases include alcoholic cardiomyopathy, congestive cardiomyopathy, hypertrophic cardiomyopathy, aortic subvalvular stenosis, pulmonary subvalvular stenosis, restrictive cardiomyopathy, Chagas cardiomyopathy, endocardial fibroelastosis, endomyocardial fibrosis, Keams Syndrome, myocardial reperfusion injury, and myocarditis.

Myocardial ischemias include coronary disease, such as angina pectoris, coronary aneurysm, coronary arteriosclerosis, coronary thrombosis, coronary vasospasm, myocardial infarction and myocardial stunning.

Cardiovascular diseases also include vascular diseases such as aneurysms, angiodysplasia, angiomatosis, bacillary angiomatosis, Hippel-Lindau Disease, Klippel-Trenaunay-Weber Syndrome, Sturge-Weber Syndrome, angioneurotic edema, aortic diseases, Takayasu's Arteritis, aortitis, Leriche's Syndrome, arterial occlusive diseases, arteritis, enarteritis, polyarteritis nodosa, cerebrovascular disorders, diabetic angiopathies, diabetic retinopathy, embolisms, thrombosis, erythromelalgia, hemorrhoids, hepatic veno-occlusive disease, hypertension, hypotension, ischemia, peripheral vascular diseases, phlebitis, pulmonary veno-occlusive disease, Raynaud's disease, CREST syndrome, retinal vein occlusion, Scimitar syndrome, superior vena cava syndrome, telangiectasia, atacia telangiectasia, hereditary hemorrhagic telangiectasia, varicocele, varicose veins, varicose ulcer, vasculitis, and venous insufficiency.

Aneurysms include dissecting aneurysms, false aneurysms, infected aneurysms, ruptured aneurysms, aortic aneurysms, cerebral aneurysms, coronary aneurysms, heart aneurysms, and iliac aneurysms.

Arterial occlusive diseases include arteriosclerosis, intermittent claudication, carotid stenosis, fibromuscular dysplasias, mesenteric vascular occlusion, Moyamoya disease, renal artery obstruction, retinal artery occlusion, and thromboangiitis obliterans.

Cerebrovascular disorders include carotid artery diseases, cerebral amyloid angiopathy, cerebral aneurysm, cerebral anoxia, cerebral arteriosclerosis, cerebral arteriovenous malformation, cerebral artery diseases, cerebral embolism and thrombosis, carotid artery thrombosis, sinus thrombosis, Wallenberg's syndrome, cerebral hemorrhage, epidural hematoma, subdural hematoma, subaraxhnoid hemorrhage, cerebral infarction, cerebral ischemia (including transient), subclavian steal syndrome, periventricular leukomalacia, vascular headache, cluster headache, migraine, and vertebrobasilar insufficiency.

Embolisms include air embolisms, amniotic fluid embolisms, cholesterol embolisms, blue toe syndrome, fat embolisms, pulmonary embolisms, and thromoboembolisms. Thrombosis include coronary thrombosis, hepatic vein thrombosis, retinal vein occlusion, carotid artery thrombosis, sinus thrombosis, Wallenberg's syndrome, and thrombophlebitis.

Ischemia includes cerebral ischemia, ischemic colitis, compartment syndromes, anterior compartment syndrome, myocardial ischemia, reperfusion injuries, and peripheral limb ischemia. Vasculitis includes aortitis, arteritis, Behcet's Syndrome, Churg-Strauss Syndrome, mucocutaneous lymph node syndrome, thromboangiitis obliterans, hypersensitivity vasculitis, Schoenlein-Henoch purpura, allergic cutaneous vasculitis, and Wegener's granulomatosis.

METH1 and/or METH2 polynucleotides or polypeptides, or agonists or antagonists of METH1 and/or METH2, are especially effective for the treatment of critical limb ischemia and coronary disease.

METH1 and/or METH2 polypeptides may be administered using any method known in the art, including, but not limited to, direct needle injection at the delivery site, intravenous injection, topical administration, catheter infusion, biolistic injectors, particle accelerators, gelfoam sponge depots, other commercially available depot materials, osmotic pumps, oral or suppositorial solid pharmaceutical formulations, decanting or topical applications during surgery, aerosol delivery. Such methods are known in the art. METH1 and/or METH2 polypeptides may be administered as part of a pharmaceutical composition, described in more detail below. Methods of delivering METH1 and/or METH2 polynucleotides are described in more detail herein.

Diseases at the Cellular Level

Diseases associated with increased cell survival or the inhibition of apoptosis that could be treated or detected by METH1 and/or METH2 polynucleotides or polypeptides, as well as antagonists or agonists of METH1 and/or METH2, include cancers (such as follicular lymphomas, carcinomas with p53 mutations, and hormone-dependent tumors, including, but not limited to colon cancer, cardiac tumors, pancreatic cancer, melanoma, retinoblastoma, glioblastoma, lung cancer, intestinal cancer, testicular cancer, stomach cancer, neuroblastoma, myxoma, myoma, lymphoma, endothelioma, osteoblastoma, osteoclastoma, osteosarcoma, chondrosarcoma, adenoma, breast cancer, prostate cancer, Kaposi's sarcoma and ovarian cancer); autoimmune disorders (such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) and viral infections (such as herpes viruses, pox viruses and adenoviruses), inflammation, graft v. host disease, acute graft rejection, and chronic graft rejection. In preferred embodiments, METH1 and/or METH2 polynucleotides, polypeptides, and/or antagonists of the invention are used to inhibit growth, progression, and/or metasis of cancers, in particular those listed above.

Additional diseases or conditions associated with increased cell survival that could be treated or detected by METH1 and/or METH2 polynucleotides or polypeptides, or agonists or antagonists of METH1 and/or METH2, include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Diseases associated with increased apoptosis that could be treated or detected by METH1 and/or METH2 polynucleotides or polypeptides, as well as agonists or antagonists of METH1 and/or METH2, include AI)S; neurodegenerative disorders (such as Alzheimer's disease, Parkinson's disease, Amyotrophic lateral sclerosis, Retinitis pigmentosa, Cerebellar degeneration and brain tumor or prior associated disease); autoimmune disorders (such as, multiple sclerosis, Sjogren's syndrome, Hashimoto's thyroiditis, biliary cirrhosis, Behcet's disease, Crohn's disease, polymyositis, systemic lupus erythematosus and immune-related glomerulonephritis and rheumatoid arthritis) myelodysplastic syndromes (such as a plastic anemia), graft v. host disease, ischemic injury (such as that caused by myocardial infarction, stroke and reperfusion injury), liver injury (e.g., hepatitis related liver injury, ischemia/reperfusion injury, cholestosis (bile duct injury) and liver cancer); toxin-induced liver disease (such as that caused by alcohol), septic shock, cachexia and anorexia.

Wound Healing and Epithelial Cell Proliferation

In accordance with yet a further aspect of the present invention, there is provided a process for utilizing METH1 and/or METH2 polynucleotides or polypeptides, as well as agonists or antagonists of METH1 and/or METH2, for therapeutic purposes, for example, to stimulate epithelial cell proliferation and basal keratinocytes for the purpose of wound healing, and to stimulate hair follicle production and healing of dermal wounds. METH1 and/or METH2 polynucleotides or polypeptides, as well as agonists or antagonists of METH1 and/or METH2, may be clinically useful in stimulating wound healing including surgical wounds, excisional wounds, deep wounds involving damage of the dermis and epidermis, eye tissue wounds, dental tissue wounds, oral cavity wounds, diabetic ulcers, dermal ulcers, cubitus ulcers, arterial ulcers, venous stasis ulcers, bums resulting from heat exposure or chemicals, and other abnormal wound healing conditions such as uremia, malnutrition, vitamin deficiencies and complications associted with systemic treatment with steroids, radiation therapy and antineoplastic drugs and antimetabolites. METH1 and/or METH2 polynucleotides or polypeptides, as well as agonists or antagonists of METH1 and/or METH2, could be used to promote dermal reestablishment subsequent to dermal loss.

METH1 and/or METH2 polynucleotides or polypeptides, as well as agonists or antagonists of METH1 and/or METH2, could be used to increase the adherence of skin grafts to a wound bed and to stimulate re-epithelialization from the wound bed. The following are types of grafts that METH1 and/or METH2 polynucleotides or polypeptides, agonists or antagonists of METH1 and/or METH2, could be used to increase adherence to a wound bed: autografts, artificial skin, allografts, autodermic graft, autoepdermic grafts, avacular grafts, Blair-Brown grafts, bone graft, brephoplastic grafts, cutis graft, delayed graft, dermic graft, epidermic graft, fascia graft, full thickness graft, heterologous graft, xenograft, homologous graft, hyperplastic graft, lamellar graft, mesh graft, mucosal graft, Ollier-Thiersch graft, omenpal graft, patch graft, pedicle graft, penetrating graft, split skin graft, thick split graft. METH1 and/or METH2 polynucleotides or polypeptides, as well as agonists or antagonists of METH1 and/or METH2, can be used to promote skin strength and to improve the appearance of aged skin.

It is believed that METH1 and/or METH2 polynucleotides or polypeptides, as well as agonists or antagonists of METH1 and/or METH2, will also produce changes in hepatocyte proliferation, and epithelial cell proliferation in the lung, breast, pancreas, stomach, small intesting, and large intestine. METH1 and/or METH2 polynucleotides or polypeptides, as well as agonists or antagonists of METH1 and/or METH2, could promote proliferation of epithelial cells such as sebocytes, hair follicles, hepatocytes, type II pneumocytes, mucin-producing goblet cells, and other epithelial cells and their progenitors contained within the skin, lung, liver, and gastrointestinal tract. METH1 and/or METH2 polynucleotides or polypeptides, agonists or antagonists of METH1 and/or METH2, may promote proliferation of endothelial cells, keratinocytes, and basal keratinocytes.

METH1 and/or METH2 polynucleotides or polypeptides, as well as agonists or antagonists of METH1 and/or METH2, could also be used to reduce the side effects of gut toxicity that result from radiation, chemotherapy treatments or viral infections. METH1 and/or METH2 polynucleotides or polypeptides, as well as agonists or antagonists of METH1 and/or METH2, may have a cytoprotective effect on the small intestine mucosa. METH1 and/or METH2 polynucleotides or polypeptides, as well as agonists or antagonists of METH1 and/or METH2, may also stimulate healing of mucositis (mouth ulcers) that result from chemotherapy and viral infections. METH1 and/or METH2 polynucleotides or polypeptides, as well as agonists or antagonists of METH1 and/or METH2, could further be used in full regeneration of skin in full and partial thickness skin defects, including bums, (i.e., repopulation of hair follicles, sweat glands, and sebaceous glands), treatment of other skin defects such as psoriasis. METH1 and/or METH2 polynucleotides or polypeptides, as well as agonists or antagonists of METH1 and/or METH2, could be used to treat epidermolysis bullosa, a defect in adherence of the epidermis to the underlying dermis which results in frequent, open and painful blisters by accelerating reepithelialization of these lesions. METH1 and/or METH2 polynucleotides or polypeptides, as well as agonists or antagonists of METH1 and/or METH2, could also be used to treat gastric and doudenal ulcers and help heal by scar formation of the mucosal lining and regeneration of glandular mucosa and duodenal mucosal lining more rapidly. Inflamamatory bowel diseases, such as Crohn's disease and ulcerative colitis, are diseases which result in destruction of the mucosal surface of the small or large intestine, respectively. Thus, METH1 and/or METH2 polynucleotides or polypeptides, as well as agonists or antagonists of METH1 and/or METH2, could be used to promote the resurfacing of the mucosal surface to aid more rapid healing and to prevent progression of inflammatory bowel disease. Treatment with METH1 and/or METH2 polynucleotides or polypeptides, agonists or antagonists of METH1 and/or METH2, is expected to have a significant effect on the production of mucus throughout the gastrointestinal tract and could be used to protect the intestinal mucosa from injurious substances that are ingested or following surgery. METH1 and/or METH2 polynucleotides or polypeptides, as well as agonists or antagonists of METH1 and/or METH2, could be used to treat diseases associate with the under expression of METH1 and/or METH2.

Moreover, METH1 and/or METH2 polynucleotides or polypeptides, as well as agonists or antagonists of METH1 and/or METH2, could be used to prevent and heal damage to the lungs due to various pathological states. A growth factor such as METH1 and/or METH2 polynucleotides or polypeptides, as well as agonists or antagonists of METH1 and/or METH2, could stimulate proliferation and differentiation and promote the repair of alveoli and brochiolar epithelium to prevent or treat acute or chronic lung damage. For example, emphysema, which results in the progressive loss of aveoli, and inhalation injuries, i.e., resulting from smoke inhalation and bums, that cause necrosis of the bronchiolar epithelium and alveoli could be effectively treated using METH1 and/or METH2 polynucleotides or polypeptides, agonists or antagonists of METH1 and/or METH2. Also, METH1 and/or METH2 polynucleotides or polypeptides, as well as agonists or antagonists of METH1 and/or METH2, could be used to stimulate the proliferation and differentiation of type II pneumocytes, which may help treat or prevent diseases such as hyaline membrane diseases, such as infant respiratory distress syndrome and bronchopulmonary displasia, in premature infants.

METH1 and/or METH12 polynucleotides or polypeptides, as well as agonists or antagonists of METH1 and/or METH2, could stimulate the proliferation and differentiation of hepatocytes and, thus, could be used to alleviate or treat liver diseases and pathologies such as fulminant liver failure caused by cirrhosis, liver damage caused by viral hepatitis and toxic substances (i.e., acetaminophen, carbon tetraholoride and other hepatotoxins known in the art).

In addition, METH1 and/or METH2 polynucleotides or polypeptides, as well as agonists or antagonists of METH1 and/or METH2, could be used treat or prevent the onset of diabetes mellitus. In patients with newly diagnosed Types I and II diabetes, where some islet cell function remains, METH1 and/or METH2 polynucleotides or polypeptides, as well as agonists or antagonists of METH1 and/or METH2, could be used to maintain the islet function so as to alleviate, delay or prevent permanent manifestation of the disease. Also, METH1 and/or METH2 polynucleotides or polypeptides, as well as agonists or antagonists of METH1 and/or METH2, could be used as an auxiliary in islet cell transplantation to improve or promote islet cell function.

Infectious Disease

METH1 and/or METH2 polynucleotides or polypeptides, or agonists or antagonists of METH1 and/or METH2, can be used to treat or detect infectious agents. For example, by increasing the immune response, particularly increasing the proliferation and differentiation of B and/or T cells, infectious diseases may be treated. The immune response may be increased by either enhancing an existing immune response, or by initiating a new immune response. Alternatively, METH1 and/or METH2 polynucleotides or polypeptides, or agonists or antagonists of METH1 and/or METH2, may also directly inhibit the infectious agent, without necessarily eliciting an immune response.

Viruses are one example of an infectious agent that can cause disease or symptoms that can be treated or detected by METH1 and/or METH2 polynucleotides or polypeptides, or agonists or antagonists of METH1 and/or METH2. Examples of viruses, include, but are not limited to the following DNA and RNA viral families: Arbovirus, Adenoviridae, Arenaviridae, Arterivirus, Birnaviridae, Bunyaviridae, Caliciviridae, Circoviridae, Coronaviridae, Flaviviridae, Hepadnaviridae (Hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picornaviridae, Poxyiridae (such as Smallpox or Vaccinia), Reoviridae (e.g., Rotavirus), Retroviridae (HTLV-, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia. METH1 and/or METH2 polynucleotides or polypeptides, or agonists or antagonists of METH1 and/or METH2, can be used to treat or detect any of these symptoms or diseases.

Similarly, bacterial or fungal agents that can cause disease or symptoms and that can be treated or detected by METH1 and/or METH2 polynucleotides or polypeptides, or agonists or antagonists of METH1 and/or METH2, include, but not limited to, the following Gram-Negative and Gram-positive bacterial families and fungi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections. METH1 and/or METH2 polynucleotides or polypeptides, or agonists or antagonists of METH1 and/or METH2, can be used to treat or detect any of these symptoms or diseases.

Moreover, parasitic agents causing disease or symptoms that can be treated or detected by METH1 and/or METH2 polynucleotides or polypeptides, or agonists or antagonists of METH1 and/or METH2, include, but are not limited to, the following families: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas. These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), Malaria, pregnancy complications, and toxoplasmosis. METH1 and/or METH2 polynucleotides or polypeptides, or agonists or antagonists of METH1 and/or METH2, can be used to treat or detect any of these symptoms or diseases.

Preferably, treatment using METH1 and/or METH2 polynucleotides or polypeptides, or agonists or antagonists of METH1 and/or METH2, could either be by administering an effective amount of METH1 and/or METH2 polypeptide to the patient, or by removing cells from the patient, supplying the cells with METH1 and/or METH2 polynucleotide, and returning the engineered cells to the patient (ex vivo therapy). Moreover, the METH1 and/or METH2 polypeptide or polynucleotide can be used as an antigen in a vaccine to raise an immune response against infectious disease.

Regeneration

METH1 and/or METH2 polynucleotides or polypeptides, or agonists or antagonists of METH1 and/or METH2, can be used to differentiate, proliferate, and attract cells, leading to the regeneration of tissues. (See, Science 276:59-87 (1997).) The regeneration of tissues could be used to repair, replace, or protect tissue damaged by congenital defects, trauma (wounds, bums, incisions, or ulcers), age, disease (e.g. osteoporosis, osteocarthritis, periodontal disease, liver failure), surgery, including cosmetic plastic surgery, fibrosis, reperfusion injury, or systemic cytokine damage.

Tissues that could be regenerated using the present invention include organs (e.g., pancreas, liver, intestine, kidney, skin, endothelium), muscle (smooth, skeletal or cardiac), vasculature (including vascular and lymphatics), nervous, hematopoietic, and skeletal (bone, cartilage, tendon, and ligament) tissue. Preferably, regeneration occurs without or decreased scarring. Regeneration also may include angiogenesis.

Moreover, METH1 and/or METH2 polynucleotides or polypeptides, or agonists or antagonists of METH1 and/or METH2, may increase regeneration of tissues difficult to heal. For example, increased tendon/ligament regeneration would quicken recovery time after damage. METH1 and/or METH2 polynucleotides or polypeptides, or agonists or antagonists of METH1 and/or METH2, of the present invention could also be used prophylactically in an effort to avoid damage. Specific diseases that could be treated include of tendinitis, carpal tunnel syndrome, and other tendon or ligament defects. A further example of tissue regeneration of non-healing wounds includes pressure ulcers, ulcers associated with vascular insufficiency, surgical, and traumatic wounds.

Similarly, nerve and brain tissue could also be regenerated by using METH1 and/or METH2 polynucleotides or polypeptides, or agonists or antagonists of METH1 and/or METH2, to proliferate and differentiate nerve cells. Diseases that could be treated using this method include central and peripheral nervous system diseases, neuropathies, or mechanical and traumatic disorders (e.g., spinal cord disorders, head trauma, cerebrovascular disease, and stoke). Specifically, diseases associated with peripheral nerve injuries, peripheral neuropathy (e.g., resulting from chemotherapy or other medical therapies), localized neuropathies, and central nervous system diseases (e.g., Alzheimer's disease, Parkinson's disease, Huntington's disease, amyotrophic lateral sclerosis, and Shy-Drager syndrome), could all be treated using the METH1 and/or METH2 polynucleotides or polypeptides, or agonists or antagonists of METH1 and/or METH2.

Chemotaxis

METH1 and/or METH2 polynucleotides or polypeptides, or agonists or antagonists of METH1 and/or METH2, may have chemotaxis activity. A chemotaxic molecule attracts or mobilizes cells (e.g., monocytes, fibroblasts, neutrophils, T-cells, mast cells, eosinophils, epithelial and/or endothelial cells) to a particular site in the body, such as a site of inflammation, infection, or hyperproliferation. The mobilized cells can then fight off and/or heal the particular trauma or abnormality.

METH1 and/or METH2 polynucleotides or polypeptides, or agonists or antagonists of METH1 and/or METH2, may increase chemotaxic activity of particular cells. These chemotactic molecules can then be used to treat inflammation, infection, hyperproliferative disorders, or any immune system disorder by increasing the number of cells targeted to a particular location in the body. For example, chemotaxic molecules can be used to treat wounds and other trauma to tissues by attracting immune cells to the injured location. As a chemotactic molecule, METH1 and/or METH2 could also attract fibroblasts, which can be used to treat wounds.

It is also contemplated that METH1 and/or METH2 polynucleotides or polypeptides, or agonists or antagonists of METH1 and/or METH2, may inhibit chemotactic activity. These molecules could also be used to treat disorders. Thus, METH1 and/or METH2 polynucleotides or polypeptides, or agonists or antagonists of METH1 and/or METH2, could be used as an inhibitor of chemotaxis.

Binding Activity

METH1 and/or METH2 polypeptides may be used to screen for molecules that bind to METH1 and/or METH2 or for molecules to which METH1 and/or METH2 binds. The binding of METH1 and/or METH2 and the molecule may activate (agonist), increase, inhibit (antagonist), or decrease activity of the METH1 and/or METH2 or the molecule bound. Examples of such molecules include antibodies, oligonucleotides, proteins (e.g., receptors), or small molecules.

Preferably, the molecule is closely related to the natural ligand of METH1 and/or METH2, e.g., a fragment of the ligand, or a natural substrate, a ligand, a structural or functional mimetic. (See, Coligan et al., Current Protocols in Immunology 1(2):Chapter 5 (1991).) Similarly, the molecule can be closely related to the natural receptor to which METH1 and/or METH2 binds, or at least, a fragment of the receptor capable of being bound by METH1 and/or METH2 (e.g., active site). In either case, the molecule can be rationally designed using known techniques.

Preferably, the screening for these molecules involves producing appropriate cells which express METH1 and/or METH2, either as a secreted protein or on the cell membrane. Preferred cells include cells from mammals, yeast, Drosophila, or E. coli. Cells expressing METH1 and/or METH2(or cell membrane containing the expressed polypeptide) are then preferably contacted with a test compound potentially containing the molecule to observe binding, stimulation, or inhibition of activity of either METH1 and/or METH2 or the molecule.

The assay may simply test binding of a candidate compound to METH1 and/or METH2, wherein binding is detected by a label, or in an assay involving competition with a labeled competitor. Further, the assay may test whether the candidate compound results in a signal generated by binding to METH1 and/or METH2.

Alternatively, the assay can be carried out using cell-free preparations, polypeptide/molecule affixed to a solid support, chemical libraries, or natural product mixtures. The assay may also simply comprise the steps of mixing a candidate compound with a solution containing METH1 and/or METH2, measuring METH1 and/or METH2/molecule activity or binding, and comparing the METH1 and/or METH2/molecule activity or binding to a standard.

Preferably, an ELISA assay can measure METH1 and/or METH2 level or activity in a sample (e.g., biological sample) using a monoclonal or polyclonal antibody. The antibody can measure METH1 and/or METH2 level or activity by either binding, directly or indirectly, to METH1 and/or METH2 or by competing with METH1 and/or METH2 for a substrate.

Additionally, the receptor to which METH1 and/or METH2 binds can be identified by numerous methods known to those of skill in the art, for example, ligand panning and FACS sorting (Coligan, et al., Current Protocols in Immun., 1(2), Chapter 5, (1991)). For example, expression cloning is employed wherein polyadenylated RNA is prepared from a cell responsive to the polypeptides, for example, NIH3T3 cells which are known to contain multiple receptors for the FGF family proteins, and SC-3 cells, and a cDNA library created from this RNA is divided into pools and used to transfect COS cells or other cells that are not responsive to the polypeptides. Transfected cells which are grown on glass slides are exposed to the polypeptide of the present invention, after they have been labelled. The polypeptides can be labeled by a variety of means including iodination or inclusion of a recognition site for a site-specific protein kinase.

Following fixation and incubation, the slides are subjected to auto-radiographic analysis. Positive pools are identified and sub-pools are prepared and re-transfected using an iterative sub-pooling and re-screening process, eventually yielding a single clones that encodes the putative receptor.

As an alternative approach for receptor identification, the labeled polypeptides can be photoaffinity linked with cell membrane or extract preparations that express the receptor molecule. Cross-linked material is resolved by PAGE analysis and exposed to X-ray film. The labeled complex containing the receptors of the polypeptides can be excised, resolved into peptide fragments, and subjected to protein microsequencing. The amino acid sequence obtained from microsequencing would be used to design a set of degenerate oligonucleotide probes to screen a cDNA library to identify the genes encoding the putative receptors.

Moreover, the techniques of gene-shuffling, motif-shuffling, exon-shuffling, and/or codon-shuffling (collectively referred to as “DNA shuffling”) may be employed to modulate the activities of METH1 or METH2 thereby effectively generating agonists and antagonists of METH1 or METH2. See generally, U.S. Pat. Nos. 5,605,793, 5,811,238, 5,830,721, 5,834,252, and 5,837,458; and Patten, P. A. et al., Curr. Opinion Biotechnol. 8:724-733 (1997); Harayama, S. Trends Biotechnol. 16(2):76-82 (1998); Hansson, L. O. et al., J. Mol. Biol. 287:265-276 (1999); and Lorenzo, M. M. and Blasco, R. Biotechniques 24(2):308-313 (1998) (each of these patents and publications are hereby incorporated by reference). In one embodiment, alteration of METH1 or METH2 polynucleotides and corresponding polypeptides may be achieved by DNA shuffling. DNA shuffling involves the assembly of two or more DNA segments into a desired METH1 or METH2 molecule by homologous, or site-specific, recombination.

In another embodiment, METH1 or METH2 polynucleotides and corresponding polypeptides may be altered by being subjected to random mutagenesis by error-prone PCR, random nucleotide insertion, or other methods prior to recombination. In another embodiment, one or more components, motifs, sections, parts, domains, fragments, etc., or METH1 or METH2 may be recombined with one or more components, motifs, sections, parts, domains, fragments, etc. of one or more heterologous molecules. In preferred embodiments, the heterologous molecule is a growth factor such as, for example, platelet-derived growth factor (PDGF), insulin-like growth factor (IGI-I), transforming growth factor (TGF)-alpha, epidermal growth factor (EGF), fibroblast growth factor (FGF), TGF-beta, bone morphogenetic protein (BMP)-2, BMP-4, BMP-5, BMP-6, BMP-6, BMP-7, activins A and B, decapentaplegic (dpp), 60A, OP-2, dorsalin, growth differentiation factors (GDFs), nodal, MIS, inhibin-alpha, TGF-beta1, TGF-beta2, TGF-beta3, TGF-beta5, and glial-derived neutrophic factor (GDNF).

Other preferred fragments are biologically active METH1 or METH2 fragments. Biologically active fragments are those exhibiting activity similar, but not necessarily identical, to an activity of the METH1 or METH2 polypeptide. The biological activity of the fragments may include an improved desired activity, or a decreased undesirable activity.

Additionally, this invention provides a method of screening compounds to identify those which modulate the action of the polypeptide of the present invention. An example of such an assay comprises combining a mammalian fibroblast cell, the polypeptide of the present invention, the compound to be screened and 3[H] thymidine under cell culture conditions where the fibroblast cell would normally proliferate. A control assay may be performed in the absence of the compound to be screened and compared to the amount of fibroblast proliferation in the presence of the compound to determine if the compound stimulates proliferation by determining the uptake of 3[H] thymidine in each case. The amount of fibroblast cell proliferation is measured by liquid scintillation chromatography which measures the incorporation of 3 [H] thymidine. Both agonist and antagonist compounds may be identified by this procedure.

In another method, a mammalian cell or membrane preparation expressing a receptor for a polypeptide of the present invention is incubated with a labeled polypeptide of the present invention in the presence of the compound. The ability of the compound to enhance or block this interaction could then be measured. Alternatively, the response of a known second messenger system following interaction of a compound to be screened and the METH1 and/or METH2 receptor is measured and the ability of the compound to bind to the receptor and elicit a second messenger response is measured to determine if the compound is a potential agonist or antagonist. Such second messenger systems include but are not limited to, cAMP guanylate cyclase, ion channels or phosphoinositide hydrolysis.

All of these above assays can be used as diagnostic or prognostic markers. The molecules discovered using these assays can be used to treat disease or to bring about a particular result in a patient (e.g., blood vessel growth) by activating or inhibiting the METH1 and/or METH2/molecule. Moreover, the assays can discover agents which may inhibit or enhance the production of METH1 and/or METH2 from suitably manipulated cells or tissues. Therefore, the invention includes a method of identifying compounds which bind to METH1 and/or METH2 comprising the steps of: (a) incubating a candidate binding compound with METH1 and/or METH2; and (b) determining if binding has occurred. Moreover, the invention includes a method of identifying agonists/antagonists comprising the steps of: (a) incubating a candidate compound with METH1 and/or METH2, (b) assaying a biological activity, and (b) determining if a biological activity of METH1 and/or METH2 has been altered.

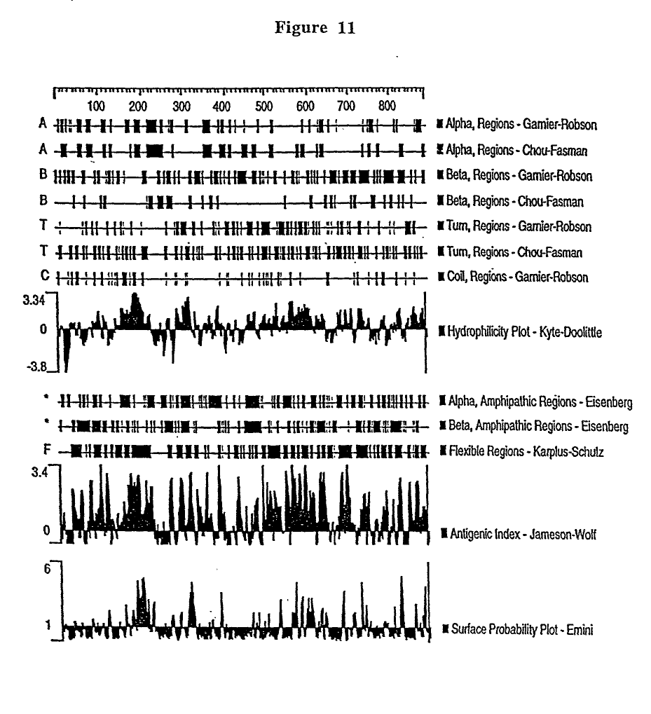

Also, one could identify molecules which bind METH1 and/or METH2 experimentally by using the beta-pleated sheet regions disclosed in FIGS. 10 and 11 and Tables 1 and 2. Accordingly, specific embodiments of the invention are directed to polynucleotides encoding polypeptides which comprise, or alternatively consist of, the amino acid sequence of each beta pleated sheet regions disclosed in FIG. 10/Table 1 and FIG. 11/Table 2. Additional embodiments of the invention are directed to polynucleotides encoding METH1 and/or METH2 polypeptides which comprise, or alternatively consist of, any combination or all of the beta pleated sheet regions disclosed in FIG. 10/Table 1 and FIG. 11/Table 2. Additional preferred embodiments of the invention are directed to polypeptides which comprise, or alternatively consist of, the METH1 and/or METH2 amino acid sequence of each of the beta pleated sheet regions disclosed in FIG. 10/Table 1 and FIG. 11/Table 2. Additional embodiments of the invention are directed to METH1 and/or METH2 polypeptides which comprise, or alternatively consist of, any combination or all of the beta pleated sheet regions disclosed in FIG. 10/Table 1 and FIG. 11/Table 2.

Antisense And Ribozyme (Antagonists)

In specific embodiments, antagonists according to the present invention are nucleic acids corresponding to the sequences contained in SEQ ID NO: 1 or 3, or the complementary strand thereof, and/or to nucleotide sequences contained in the deposited clones. In one embodiment, antisense sequence is generated internally by the organism, in another embodiment, the antisense sequence is separately administered (see, for example, O'Connor, J., Neurochem. 56:560 (1991). Oligodeoxynucleotides as Anitsense Inhibitors of Gene Expression, CRC Press, Boca Raton, Fla. (1988). Antisense technology can be used to control gene expression through antisense DNA or RNA, or through triple-helix formation. Antisense techniques are discussed for example, in Okano, J., Neurochem. 56:560 (1991); Oligodeoxynucleotides as Antisense Inhibitors of Gene Expression, CRC Press, Boca Raton, Fla. (1988). Triple helix formation is discussed in, for instance, Lee et al., Nucleic Acids Research 6:3073 (1979); Cooney et al., Science 241:456 (1988); and Dervan et al., Science 251:1300 (1991). The methods are based on binding of a polynucleotide to a complementary DNA or RNA.

For example, the 5′ coding portion of a polynucleotide that encodes the mature polypeptide of the present invention may be used to design an antisense RNA oligonucleotide of from about 10 to 40 base pairs in length. A DNA oligonucleotide is designed to be complementary to a region of the gene involved in transcription thereby preventing transcription and the production of the receptor. The antisense RNA oligonucleotide hybridizes to the mRNA in vivo and blocks translation of the mRNA molecule into receptor polypeptide.

In one embodiment, the METH1 and/or METH2 antisense nucleic acid of the invention is produced intracellularly by transcription from an exogenous sequence. For example, a vector or a portion thereof, is transcribed, producing an antisense nucleic acid (RNA) of the invention. Such a vector would contain a sequence encoding the METH1 and/or METH2 antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others know in the art, used for replication and expression in vertebrate cells. Expression of the sequence encoding METH1 and/or METH2, or fragments thereof, can be by any promoter known in the art to act in vertebrate, preferably human cells. Such promoters can be inducible or constitutive. Such promoters include, but are not limited to, the SV40 early promoter region (Bernoist and Chambon, Nature 29:304-310 (1981), the promoter contained in the 3′ long terminal repeat of Rous sarcoma virus (Yamamoto et al., Cell 22:787-797 (1980), the herpes thymidine promoter (Wagner et al., Proc. Natl. Acad. Sci. U.S.A. 78:1441-1445 (1981), the regulatory sequences of the metallothionein gene (Brinster, et al., Nature 296:39-42 (1982)), etc.

The antisense nucleic acids of the invention comprise a sequence complementary to at least a portion of an RNA transcript of a METH1 and/or METH2 gene. However, absolute complementarity, although preferred, is not required. A sequence “complementary to at least a portion of an RNA,” referred to herein, means a sequence having sufficient complementarity to be able to hybridize with the RNA, forming a stable duplex; in the case of double stranded METH1 and/or METH2 antisense nucleic acids, a single strand of the duplex DNA may thus be tested, or triplex formation may be assayed. The ability to hybridize will depend on both the degree of complementarity and the length of the antisense nucleic acid Generally, the larger the hybridizing nucleic acid, the more base mismatches with a METH1 and/or METH2 RNA it may contain and still form a stable duplex (or triplex as the case may be). One skilled in the art can ascertain a tolerable degree of mismatch by use of standard procedures to determine the melting point of the hybridized complex.

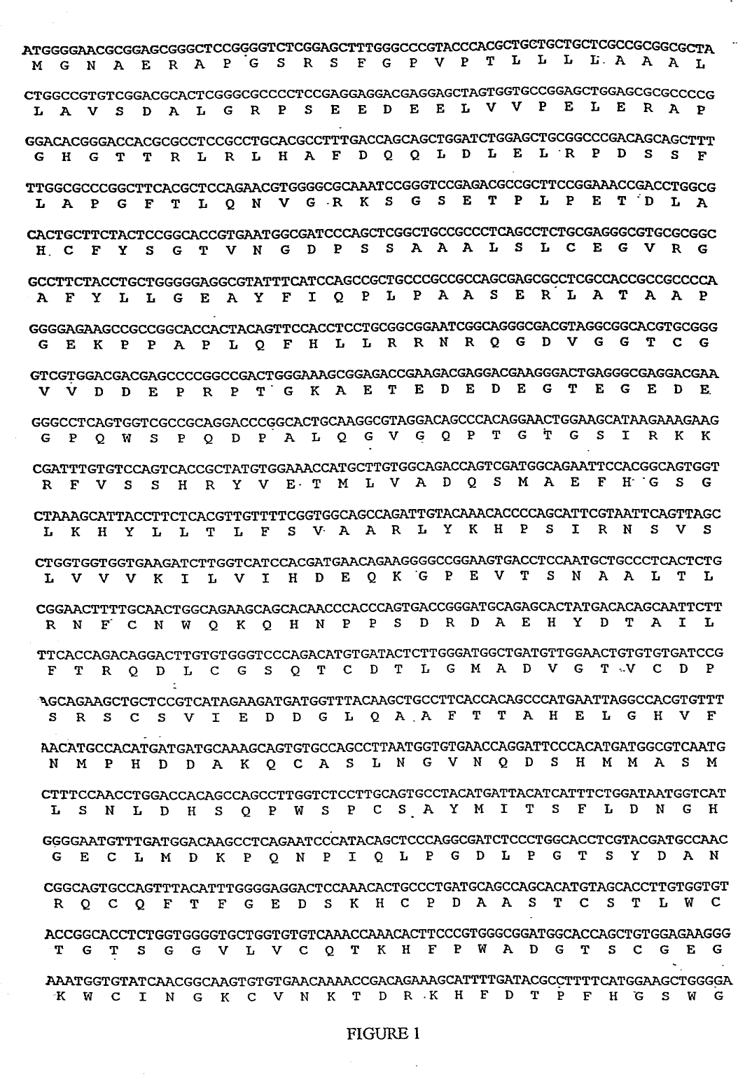

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. See generally, Wagner, R., 1994, Nature 372:333-335. Thus, oligonucleotides complementary to either the 5′- or 3′- non-translated, non-coding regions of METH1 and/or METH2 shown in FIG. 1 could be used in an antisense approach to inhibit translation of endogenous METH1 and/or METH2 mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of METH1 and/or METH2 mRNA, antisense nucleic acids should be at least six nucleotides in length, and are preferably oligonucleotides ranging from 6 to about 50 nucleotides in length. In specific aspects the oligonucleotide is at least 10 nucleotides, at least 17 nucleotides, at least 25 nucleotides or at least 50 nucleotides.

The polynucleotides of the invention can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors in vivo), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88109810, published Dec. 15, 1988) or the blood-brain barrier (see, e.g., PCT Publication No. WO89/10134, published Apr. 25, 1988), hybridization-triggered cleavage agents. (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group including, but not limited to, 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine,

5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including, but not limited to, arabinose, 2-fluoroarabinose, xylulose, and hexose.

In yet another embodiment, the antisense oligonucleotide comprises at least one modified phosphate backbone selected from the group including, but not limited to, a phosphorothioate, a phosphorodithioate, a phosphoramidothioate, a phosphoramidate, a phosphordiamidate, a methylphosphonate, an alkyl phosphotriester, and a formacetal or analog thereof.

In yet another embodiment, the antisense oligonucleotide is an a-anomeric oligonucleotide. An a-anomeric oligonucleotide forms specific double-stranded hybrids with complementary RNA in which, contrary to the usual b-units, the strands run parallel to each other (Gautier et al., 1987, Nucl. Acids Res. 15:6625-6641). The oligonucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330). Polynucleotides of the invention may be synthesized by standard methods known in the art, e.g. by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

While antisense nucleotides complementary to the METH1 and/or METH2 coding region sequence could be used, those complementary to the transcribed untranslated region are most preferred.

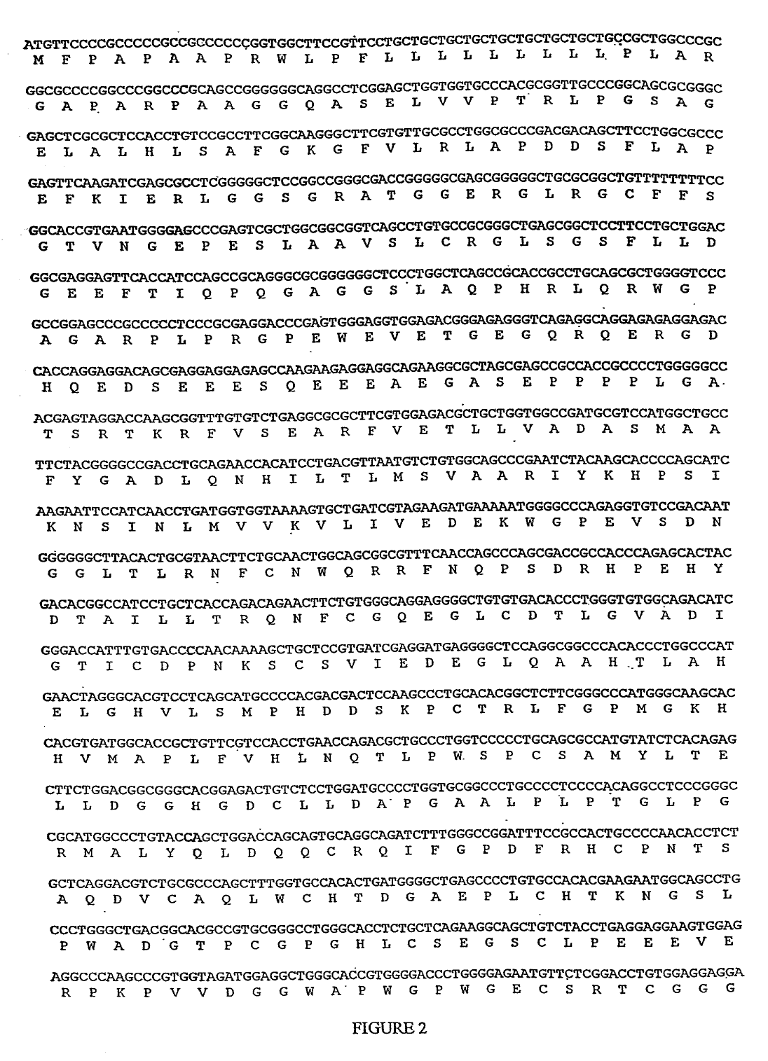

Potential antagonists according to the invention also include catalytic RNA, or a ribozyme (See, e.g., PCT International Publication WO 90/11364, published Oct. 4, 1990; Sarver et al., Science 247:1222-1225 (1990). While ribozymes that cleave mRNA at site specific recognition sequences can be used to destroy METH1 and/or METH2 mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334:585-591 (1988). There are numerous potential hammerhead ribozyme cleavage sites within the nucleotide sequence of METH1 and/or METH2 (FIGS. 1 and 2). Preferably, the ribozyme is engineered so that the cleavage recognition site is located near the 5′ end of the METH1 and/or METH2 mRNA; i.e., to increase efficiency and minimize the intracellular accumulation of non-functional mRNA transcripts.

As in the antisense approach, the ribozymes of the invention can be composed of modified oligonucleotides (e.g. for improved stability, targeting, etc.) and should be delivered to cells which express METH1 and/or METH2 in vivo. DNA constructs encoding the ribozyme may be introduced into the cell in the same manner as described above for the introduction of antisense encoding DNA. A preferred method of delivery involves using a DNA construct “encoding” the ribozyme under the control of a strong constitutive promoter, such as, for example, pol III or pol II promoter, so that transfected cells will produce sufficient quantities of the ribozyme to destroy endogenous METH1 and/or METH2 messages and inhibit translation. Since ribozymes, unlike antisense molecules, are catalytic, a lower intracellular concentration is required for efficiency.

Antagonist/agonist compounds may be employed to inhibit the cell growth and proliferation effects of the polypeptides of the present invention on neoplastic cells and tissues, i.e. stimulation of angiogenesis of tumors, and, therefore, retard or prevent abnormal cellular growth and proliferation, for example, in tumor formation or growth.

The antagonist/agonist may also be employed to prevent hyper-vascular diseases, and prevent the proliferation of epithelial lens cells after extracapsular cataract surgery. Prevention of the mitogenic activity of the polypeptides of the present invention may also be desirous in cases such as restenosis after balloon angioplasty.

The antagonist/agonist may also be employed to prevent the growth of scar tissue during wound healing.

The antagonist/agonist may also be employed to treat the diseases described herein.

Other Activities

As stated below, METH1 and METH2 share structural and sequence homology with memebrs of the ADAM family. ADAM proteins have been shown to proteolytically process membrane-anchored proteins, including TNF (Black et al., Nature 385:729 (1997); Moss et al., Nature 385:733 (1997)). Thus, METH1 and/or METH2 may be useful in proteolytic processing of membrane-anchored proteins. Membrane-anchored proteins which may be proteolytically processed by METH1 and/or METH2 include cytokines, growth factors, cytokine receptors and growth factor receptors.

METH1 or METH2 polypeptides or polynucleotides, or agonists or antagonists of METH1 and/or METH2, may also increase or decrease the differentiation or proliferation of embryonic stem cells, besides, as discussed above, hematopoietic lineage.

METH1 or METH2 polypeptides or polynucleotides, or agonists or antagonists of METH1 and/or METH2, may also be used to modulate mammalian characteristics, such as body height, weight, hair color, eye color, skin, percentage of adipose tissue, pigmentation, size, and shape (e.g., cosmetic surgery). Similarly, METH1 or METH2 polypeptides or polynucleotides, or agonists or antagonists of METH1 and/or METH2, may be used to modulate mammalian metabolism affecting catabolism, anabolism, processing, utilization, and storage of energy.

As angiogenesis is a key factor in supporting adipose tissue, METH1 or METH2 polypeptides or polynucleotides, or agonists or antagonists of METH1 and/or METH2 may be used to control weight, reduce weight, treat obesity, and/or control adipose tissue in an individual.

METH1 or METH2 polypeptides or polynucleotides, or agonists or antagonists of METH1 and/or METH2, may be used to change a mammal's mental state or physical state by influencing biorhythms, circadian rhythms, depression (including depressive disorders), tendency for violence, tolerance for pain, reproductive capabilities (preferably by Activin or Inhibin-like activity), hormonal or endocrine levels, appetite, libido, memory, stress, or other cognitive qualities.

METH1 or METH2 polypeptides or polynucleotides, or agonists or antagonists of METH1 and/or METH2, may also be used as a food additive or preservative, such as to increase or decrease storage capabilities, fat content, lipid, protein, carbohydrate, vitamins, minerals, cofactors or other nutritional components.

The above-recited applications have uses in a wide variety of hosts. Such hosts include, but are not limited to, human, murine, rabbit, goat, guinea pig, camel, horse, mouse, rat, hamster, pig, micro-pig, chicken, goat, cow, sheep, dog, cat, non-human primate, and human. In specific embodiments, the host is a mouse, rabbit, goat, guinea pig, chicken, rat, hamster, pig, sheep, dog or cat. In preferred embodiments, the host is a mammal. In most preferred embodiments, the host is a human.

Anti-angiogenesis

As shown in Examples 4 and 5, METH1 and METH2 inhibit angiogenesis. Thus, the present invention provides a method of treating an angiogenesis-related disease and/or disorder, comprising administering to an individual in need thereof a therapeutically effective amount of a METH1 and/or METH2 polynucleotide, polypeptide, and/or agonist.

For example, METH1 and/or METH2 polynucleotides, polypeptides, and/or agonists may be utilized in a variety of additional methods in order to therapeutically treat a cancer or tumor. Cancers which may be treated with METH1 and/or METH2 polynucleotides, polypeptides and/or agonists include, but are not limited to solid tumors, including prostate, lung, breast, ovarian, stomach, pancreas, larynx, esophagus, testes, liver, parotid, biliary tract, colon, rectum, cervix, uterus, endometrium, kidney, bladder, thyroid cancer; primary tumors and metastases; melanomas; glioblastoma; Kaposi's sarcoma; leiomyosarcoma; non-small cell lung cancer; colorectal cancer; advanced malignancies; and blood born tumors such as leukemias. For example, METH1 and/or METH2 polynucleotides, polypeptides, and/or agonists may be delivered topically, in order to treat cancers such as skin cancer, head and neck tumors, breast tumors, and Kaposi's sarcoma. Within yet other aspects, METH1 and/or METH2 polynucleotides, polypeptides, and/or agonists may be utilized to treat superficial forms of bladder cancer by, for example, intravesical administration. METH1 and/or METH2 polynucleotides, polypeptides and/or agonists may be delivered directly into the tumor, or near the tumor site, via injection or a catheter. Of course, as the artisan of ordinary skill will appreciate, the appropriate mode of administration will vary according to the cancer to be treated. Other modes of delivery are discussed herein.

METH1 and/or METH2 polynucleotides, polypeptides and/or agonists may be useful in treating other disorders, besides cancers, which involve angiogenesis. These disorders include, but are not limited to: benign tumors, for example hemangiomas, acoustic neuromas, neurofibromas, trachomas, and pyogenic granulomas; artheroscleric plaques; ocular angiogenic diseases, for example, diabetic retinopathy, retinopathy of prematurity, macular degeneration, corneal graft rejection, neovascular glaucoma, retrolental fibroplasia, rubeosis, retinoblastoma, uvietis and Pterygia (abnormal blood vessel growth) of the eye; rheumatoid arthritis; psoriasis; delayed wound healing; endometriosis; vasculogenesis; granulations; hypertrophic scars (keloids); nonunion fractures; scleroderma; trachoma; vascular adhesions; myocardial angiogenesis; coronary collaterals; cerebral collaterals; arteriovenous malformations; ischemic limb angiogenesis; Osler-Webber Syndrome; plaque neovascularization; telangiectasia; hemophiliac joints; angiofibroma; fibromuscular dysplasia; wound granulation; Crohn's disease; and atherosclerosis.

For example, within one aspect of the present invention methods are provided for treating hypertrophic scars and keloids, comprising the step of administering a METH1 and/or METH2 polynucleotide, polypeptide, and/or agonist to a hypertrophic scar or keloid. Within one embodiment of the present invention METH1 and/or METH2 polynucleotides, polypeptides, and/or agonists are directly injected into a hypertrophic scar or keloid, in order to prevent the progression of these lesions. This therapy is of particular value in the prophylactic treatment of conditions which are known to result in the development of hypertrophic scars and keloids (e.g., burns), and is preferably initiated after the proliferative phase has had time to progress (approximately 14 days after the initial injury), but before hypertrophic scar or keloid development.

As noted above, the present invention also provides methods for treating neovascular diseases of the eye, including for example, corneal neovascularization, neovascular glaucoma, proliferative diabetic retinopathy, retrolental fibroplasia and macular degeneration, as well as other eye inflammatory diseases, ocular tumors and diseases associated with choroidal or iris neovascularization. See, e.g., reviews by Waltman et al., Am. J. Ophthal 85:704-710 (1978) and Gartner et al., Surv. Ophthal. 22:291-312 (1978).

Thus, within one aspect of the present invention methods are provided for treating neovascular diseases of the eye such as corneal neovascularization (including corneal graft neovascularization), comprising the step of administering to a patient a therapeutically effective amount of a METH1 and/or METH2 compound (as described above) to the cornea, such that the formation of blood vessels is inhibited. Briefly, the cornea is a tissue which normally lacks blood vessels. In certain pathological conditions however, capillaries may extend into the cornea from the pericorneal vascular plexus of the limbus. When the cornea becomes vascularized, it also becomes clouded, resulting in a decline in the patient's visual acuity. Visual loss may become complete if the cornea completely opacitates.

A wide variety of disorders can result in corneal neovascularization, including for example, corneal infections (e.g., trachoma, herpes simplex keratitis, leishmaniasis and onchocerciasis), immunological processes (e.g., graft rejection and Stevens-Johnson's syndrome), alkali burns, trauma, inflammation (of any cause), toxic and nutritional deficiency states, and as a complication of wearing contact lenses.

Within particularly preferred embodiments of the invention, METH1 and/or METH2 may be prepared for topical administration in saline (combined with any of the preservatives and antimicrobial agents commonly used in ocular preparations), and administered in eyedrop form. The solution or suspension may be prepared in its pure form and administered several times daily. Alternatively, anti-angiogenic compositions, prepared as described above, may also be administered directly to the cornea. Within preferred embodiments, the anti-angiogenic composition is prepared with a muco-adhesive polymer which binds to cornea. Within further embodiments, the anti-angiogenic factors or anti-angiogenic compositions may be utilized as an adjunct to conventional steroid therapy.

Topical therapy may also be useful prophylactically in corneal lesions which are known to have a high probability of inducing an angiogenic response (such as chemical burns). In these instances the treatment, likely in combination with steroids, may be instituted immediately to help prevent subsequent complications.

Within other embodiments, the compounds described above may be injected directly into the corneal stroma by an ophthalmologist under microscopic guidance. The preferred site of injection may vary with the morphology of the individual lesion, but the goal of the administration would be to place the composition at the advancing front of the vasculature (i.e., interspersed between the blood vessels and the normal cornea). In most cases this would involve perilimbic corneal injection to “protect” the cornea from the advancing blood vessels. This method may also be utilized shortly after a corneal insult in order to prophylactically prevent corneal neovascularization. In this situation the material could be injected in the perilimbic cornea interspersed between the corneal lesion and its undesired potential limbic blood supply. Such methods may also be utilized in a similar fashion to prevent capillary invasion of transplanted corneas. In a sustained-release form injections might only be required 2-3 times per year. A steroid could also be added to the injection solution to reduce inflammation resulting from the injection itself.

Within another aspect of the present invention, methods are provided for treating neovascular glaucoma, comprising the step of administering to a patient a therapeutically effective amount of a METH1 and/or METH2 polypeptide, polynucleotide, and/or agonist to the eye, such that the formation of blood vessels is inhibited. In one embodiment, the compound may be administered topically to the eye in order to treat early forms of neovascular glaucoma. Within other embodiments, the compound may be implanted by injection into the region of the anterior chamber angle. Within other embodiments, the compound may also be placed in any location such that the compound is continuously released into the aqueous humor.

Within another aspect of the present invention, methods are provided for treating proliferative diabetic retinopathy, comprising the step of administering to a patient a therapeutically effective amount of a METH1 and/or METH2 polynucleotide, polypeptide, and/or agonist to the eyes, such that the formation of blood vessels is inhibited. Within particularly preferred embodiments of the invention, proliferative diabetic retinopathy may be treated by injection into the aqueous humor or the vitreous, in order to increase the local concentration of the METH1 and/or METH2 polynucleotide, polypeptide, and/or agonist in the retina. Preferably, this treatment should be initiated prior to the acquisition of severe disease requiring photocoagulation.

Within another aspect of the present invention, methods are provided for treating retrolental fibroplasia, comprising the step of administering to a patient a therapeutically effective amount of a METH1 and/or METH2 polynucleotide, polypeptide, and/or agonist to the eye, such that the formation of blood vessels is inhibited. The compound may be administered topically, via intravitreous injection and/or via intraocular implants.

METH1 and/or METH2 polynucleotides, polypeptides and/or agonists may be used to treat diseases that have angiogenesis as a pathologic consequence such as cat scratch disease (Rochele minalia quintosa), ulcers (Helicobacter pylori), Bartonellosis and bacillary angiomatosis.

METH1 and/or METH2 polynucleotides, polypeptides and/or agonists may be used as a birth control agent by preventing vascularization required for embryo implantation. In one aspect of the birth control method, an amount of the compound sufficient to block embryo implantation is administered before or after intercourse and fertilization have occurred, thus providing an effective method of birth control, possibly a “morning after” method.

METH1 and/or METH2 polynucleotides, polypeptides and/or agonists may also be used in controlling menstruation or administered as either a peritoneal lavage fluid or for peritoneal implantation in the treatment of endometriosis.

METH1 and/or METH2 polynucleotides, polypeptides, and/or agonists of the present invention may be incorporated into surgical sutures in order to prevent stitch granulomas.

METH1 and/or and METH2 polynucleotides, polypeptides, and/or agonists may be utilized in a wide variety of surgical procedures. For example, within one aspect of the present invention a METH1 and/or METH2 compositions (in the form of, for example, a spray or film) may be utilized to coat or spray an area prior to removal of a tumor, in order to isolate normal surrounding tissues from malignant tissue, and/or to prevent the spread of disease to surrounding tissues. Within other aspects of the present invention, METH1 and/or METH2 compositions (e.g., in the form of a spray) may be delivered via endoscopic procedures in order to coat tumors, or inhibit angiogenesis in a desired locale. Within yet other aspects of the present invention, surgical meshes which have been coated with anti-angiogenic compositions of the present invention may be utilized in any procedure wherein a surgical mesh might be utilized. For example, within one embodiment of the invention a surgical mesh laden with an anti-angiogenic composition may be utilized during abdominal cancer resection surgery (e.g., subsequent to colon resection) in order to provide support to the structure, and to release an amount of the anti-angiogenic factor.

Within further aspects of the present invention, methods are provided for treating tumor excision sites, comprising administering a METH1 and/or METH2 polynucleotide, polypeptide, and/or agonist to the resection margins of a tumor subsequent to excision, such that the local recurrence of cancer and the formation of new blood vessels at the site is inhibited. Within one embodiment of the invention, the anti-angiogenic compound is administered directly to the tumor excision site (e.g., applied by swabbing, brushing or otherwise coating the resection margins of the tumor with the anti-angiogenic compound). Alternatively, the anti-angiogenic compounds may be incorporated into known surgical pastes prior to administration. Within particularly preferred embodiments of the invention, the anti-angiogenic compounds are applied after hepatic resections for malignancy, and after neurosurgical operations.

Within one aspect of the present invention, METH1 and/or METH2 polynucleotides, polypeptides, and/or agonists may be administered to the resection margin of a wide variety of tumors, including for example, breast, colon, brain and hepatic tumors. For example, within one embodiment of the invention, anti-angiogenic compounds may be administered to the site of a neurological tumor subsequent to excision, such that the formation of new blood vessels at the site are inhibited.

The METH1 and/or METH2 polynucleotides, polypeptides, and/or agonists of the present invention may also be administered along with other anti-angiogenic factors. Representative examples of other anti-angiogenic factors include: Anti-Invasive Factor, retinoic acid and derivatives thereof, paclitaxel, Suramin, Tissue Inhibitor of Metalloproteinase-1, Tissue Inhibitor of Metalloproteinase-2, Plasminogen Activator Inhibitor-1, Plasminogen Activator Inhibitor-2, and various forms of the lighter “d group” transition metals.

Lighter “d group” transition metals include, for example, vanadium, molybdenum, tungsten, titanium, niobium, and tantalum species. Such transition metal species may form transition metal complexes. Suitable complexes of the above-mentioned transition metal species include oxo transition metal complexes.

Representative examples of vanadium complexes include oxo vanadium complexes such as vanadate and vanadyl complexes. Suitable vanadate complexes include metavanadate and orthovanadate complexes such as, for example, ammonium metavanadate, sodium metavanadate, and sodium orthovanadate. Suitable vanadyl complexes include, for example, vanadyl acetylacetonate and vanadyl sulfate including vanadyl sulfate hydrates such as vanadyl sulfate mono- and trihydrates.

Representative examples of tungsten and molybdenum complexes also include oxo complexes. Suitable oxo tungsten complexes include tungstate and tungsten oxide complexes. Suitable tungstate complexes include ammonium tungstate, calcium tungstate, sodium tungstate dihydrate, and tungstic acid. Suitable tungsten oxides include tungsten (IV) oxide and tungsten (VI) oxide. Suitable oxo molybdenum complexes include molybdate, molybdenum oxide, and molybdenyl complexes. Suitable molybdate complexes include ammonium molybdate and its hydrates, sodium molybdate and its hydrates, and potassium molybdate and its hydrates. Suitable molybdenum oxides include molybdenum (VI) oxide, molybdenum (VI) oxide, and molybdic acid. Suitable molybdenyl complexes include, for example, molybdenyl acetylacetonate. Other suitable tungsten and molybdenum complexes include hydroxo derivatives derived from, for example, glycerol, tartaric acid, and sugars.

A wide variety of other anti-angiogenic factors may also be utilized within the context of the present invention. Representative examples include platelet factor 4; protamine sulphate; sulphated chitin derivatives (prepared from queen crab shells), (Murata et al., Cancer Res. 51:22-26, 1991); Sulphated Polysaccharide Peptidoglycan Complex (SP-PG) (the function of this compound may be enhanced by the presence of steroids such as estrogen, and tamoxifen citrate); Staurosporine; modulators of matrix metabolism, including for example, proline analogs, cishydroxyproline, d,L-3,4-dehydroproline, Thiaproline, α,α-dipyridyl, β-aminopropionitrile fumarate; 4-propyl-5-(4-pyridinyl)-2(3H)-oxazolone; Methotrexate; Mitoxantrone; Heparin; Interferons; 2 Macroglobulin-serum; ChIMP-3 (Pavloff et al., J. Bio. Chem. 267:17321-17326,1992); Chymostatin (Tomkinson et al., Biochem J. 286:475480, 1992); 6-Cyclodextrin Tetradecasulfate; Eponemycin; Camptothecin; Fumagillin (Ingber et al., Nature 348:555-557, 1990); Gold Sodium Thiomalate (“GST”; Matsubara and Ziff, J. Clin. Invest. 79:1440-1446,1987); β-1-anticollagenase-serum; α2-antiplasmin (Holmes et al., J. Biol. Chem. 262(4):1659-1664, 1987); Bisantrene (National Cancer Institute); Lobenzarit disodium (N-(2)-carboxyphenyl-4-chloroanthronilic acid disodium or “CCA”; Takeuchi et al., Agents Actions 36:312-316, 1992); Thalidomide; Angostatic steroid; AGM-1470; carboxynaminolmidazole; and metalloproteinase inhibitors such as BB94.

Diagnostic Methods

The invention also relates to the use of METH1 or METH2 polynucleotides for use as diagnostic reagents. Detection of a mutated form of the METH1 or METH2 gene associated with a dysfunction will provide a diagnostic tool that can add to or define a diagnosis of a disease or susceptibility to a disease which results from under-expression, overexpression or altered expression of METH1 or METH2. Individuals carrying mutations in the METH1 or METH2 gene may be detected at the DNA level by a variety of techniques.

Nucleic acids for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in similar fashion. Deletions and insertions can be detected by a change in size of the amplified product in comparison to the normal genotype. Point mutations can be identified by hybridizing amplified DNA to labeled METH1 or METH2 nucleotide sequences. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, e.g., Myers et al., Science 230:1242 (1985). Sequence changes at specific locations may also be revealed by nuclease protection assays, such as RNase and S1 protection or the chemical cleavage method. See Cotton et al., Proc Natl Acad Sci USA 85:4397-4401 (1985). In another embodiment, an array of oligonucleotides probes comprising METH1 or METH2 nucleotide sequence or fragments thereof can be constructed to conduct efficient screening of e.g., genetic mutations. Array technology methods are well known and have general applicability and can be used to address a variety of questions in molecular genetics including gene expression, genetic linkage, and genetic variability. (See, for example, M. Chee et al., Science 274:610-613 (1996).

The diagnostic assays offer a process for diagnosing or determining a susceptibility to angiogenic diseases (cancer, cancer metastasis, chronic inflammatory disorders, rheumatoid arthritis, altherosclerosis, macular degeneration, diabetic retinopathy), restenosis, Alzheimer's disease and tissue remodeling through detection of mutation in the METH1 or METH2 gene by the methods described.

In addition, angiogenic diseases (cancer, cancer metastasis, chronic inflammatory disorders, rheumatoid arthritis, altherosclerosis, macular degeneration, diabetic retinopathy), restenosis, Alzheimer's disease and tissue remodeling can be diagnosed by methods comprising determining from a sample derived from a subject an abnormally decreased or increased level of the METH1 or METH2 polypeptide or METH1 or METH2 mRNA. Decreased or increased expression can be measured at the RNA level using any of the methods well known in the art for the quantitation of polynucleotides, such as, for example, PCR, RT-PCR, RNase protection, Northern blotting and other hybridization methods. Assay techniques that can be used to determine levels of a protein, such as an METH1 or METH2 polypeptide, in a sample derived from a host are well-known to those of skill in the art. Such assay methods include radioimmunoassays, competitive-binding assays, Western Blot analysis and ELISA assays.

Cancer Diagnosis and Prognosis

It is believed that certain tissues in mammals with cancer express significantly diminished levels of the METH1 or METH2 protein and mRNA encoding the METH1 or METH2 protein when compared to a corresponding “standard” mammal, i.e., a mammal of the same species not having the cancer. Further, it is believed that diminished levels of the METH1 or METH2 protein can be detected in certain body fluids (e.g., sera, plasma, urine, and spinal fluid) from mammals with cancer when compared to sera from mammals of the same species not having the cancer. Thus, the invention provides a diagnostic method useful during tumor diagnosis, which involves assaying the expression level of the gene encoding the METH1 protein in mammalian cells or body fluid and comparing the gene expression level with a standard METH1 gene expression level, whereby a decrease in the gene expression level under the standard is indicative of certain tumors. The invention also provides a diagnostic method useful during tumor diagnosis, which involves assaying the expression level of the gene encoding the METH2 protein in mammalian cells or body fluid and comparing the gene expression level with a standard METH2 gene expression level, whereby a decrease in the gene expression level under the standard is indicative of certain tumors.

Where a tumor diagnosis has already been made according to conventional methods, the present invention is useful as a prognostic indicator, whereby patients exhibiting diminished METH1 or METH2 gene expression will experience a worse clinical outcome relative to patients expressing the gene at a lower level.

By “assaying the expression level of the gene encoding the METH1 or METH2 protein” is intended qualitatively or quantitatively measuring or estimating the level of the METH1 or METH2 protein or the level of the mRNA encoding the METH1 or METH2 protein in a first biological sample either directly (e.g., by determining or estimating absolute protein level or mRNA level) or relatively (e.g., by comparing to the METH1 or METH2 protein level or mRNA level in a second biological sample).

Preferably, the METH1 or METH2 protein level or mRNA level in the first biological sample is measured or estimated and compared to a standard METH1 or METH2 protein level or mRNA level, the standard being taken from a second biological sample obtained from an individual not having the cancer. As will be appreciated in the art, once a standard METH1 or METH2 protein level or mRNA level is known, it can be used repeatedly as a standard for comparison.

By “biological sample” is intended any biological sample obtained from an individual, cell line, tissue culture, or other source which contains METH1 or METH2 protein or mRNA. Biological samples include mammalian body fluids (such as sera, plasma, urine, synovial fluid and spinal fluid) which contain secreted mature METH1 or METH2 protein, and adrenal, thyroid, stomach, brain, heart, placenta, lung, liver, muscle, kidney, pancreas, testis and ovarian tissue (for METH1); and prostate, small intestine, colon, brain and lung tissue (for METH2).

The present invention is useful for detecting cancer in mammals. In particular the invention is useful during diagnosis of the of following types of cancers in mammals: breast, ovarian, prostate, liver, lung, pancreatic, colon, and testicular. Preferred mammals include monkeys, apes, cats, dogs, cows, pigs, horses, rabbits and humans. Particularly preferred are humans.

Total cellular RNA can be isolated from a biological sample using the single-step guanidinium-thiocyanate-phenol-chloroform method described in Chomczynski and Sacchi, Anal. Biochem. 162:156-159(1987). Levels of mRNA encoding the METH1 or METH2 protein are then assayed using any appropriate method. These include Northern blot analysis (Harada et al., Cell 63:303-312 (1990)), S I nuclease mapping (Fujita et al., Cell 49:357-367 (1987)), the polymerase chain reaction (PCR), reverse transcription in combination with the polymerase chain reaction (RT-PCR) (Makino et al., Technique 2:295-301 (1990)), and reverse transcription in combination with the ligase chain reaction (RT-LCR).

Assaying METH1 or METH2 protein levels in a biological sample can occur using antibody-based techniques. For example, METH1 or METH2 protein expression in tissues can be studied with classical immunohistological methods (Jalkanen, M., et al., J. Cell. Biol. 101:976-985 (1985); Jalkanen, M., et al., J. Cell. Biol. 105:3087-3096 (1987)).

Other antibody-based methods useful for detecting METH1 or METH2 protein gene expression include immunoassays, such as the enzyme linked immunosorbent assay (ELISA) and the radioimmunoassay (RIA).

Suitable labels are known in the art and include enzyme labels, such as, glucose oxidase, and radioisotopes, such as iodine (¹²⁵I, ¹²¹I), carbon (¹⁴C), sulfur (³⁵S), tritium (³H), indium (¹¹²In), and technetium (^{99m}Tc), and fluorescent labels, such as fluorescein and rhodamine, and biotin.

Vaccines

Another aspect of the invention relates to a method for inducing an immunological response in a mammal which comprises inoculating the mammal with METH1 or METH2 polypeptide, or a fragment thereof, adequate to produce antibody and/or T cell immune response to protect said animal from angiogenic diseases (cancer, cancer metastasis, chronic inflammatory disorders, rheumatoid arthritis, altherosclerosis, macular degeneration, diabetic retinopathy), restenosis, Alzheimer's disease and tissue remodeling, among others. Yet another aspect of the invention relates to a method of inducing immunological response in a mammal which comprises delivering METH1 or METH2 polypeptide via a vector directing expression of METH1 or METH2 polynucleotide in vivo in order to induce such an immunological response to produce antibody to protect such animal from diseases.

Further aspect of the invention relates to an immunological/vaccine formulation (composition) which, when introduced into a mammalian host, induces an immunological response in that mammal to a METH1 or METH2 polypeptide wherein the composition comprises a METH1 or METH2 polypeptide or METH1 or METH2 gene. The vaccine formulation may further comprise a suitable carrier. Since METH1 or METH2 polypeptide may be broken down in the stomach, it is preferably administered parenterally (including subcutaneous, intramuscular, intravenous, intradermal etc. injection). Formulations suitable for parenteral administration include aqueous and non-aqueous sterile injection solutions which may contain anti-oxidants, buffers, bacteriostats and solutes which render the formulation isotonic with the blood of the recipient; and aqueous and non-aqueous sterile suspensions which may include suspending agents or thickening agents. The formulations may be presented in unit-dose or multi-dose containers, for example, sealed ampules and vials and may be stored in a freeze-dried condition requiring only the addition of the sterile liquid carrier immediately prior to use. The vaccine formulation may also include adjuvant systems for enhancing the immunogenicity of the formulation, such as oil-in water systems and other systems known in the art. The dosage will depend on the specific activity of the vaccine and can be readily determined by routine experimentation.

Modes of administration

It is recognized than an increase in the vascular supply plays a central role in tumor progression and metastasis; therefore, inhibitors of angiogenesis can prove effective as adjuvant therapy for cancer patients. Some of the currently recognized angiogenic suppressors are poor candidates for systemic treatment due to severe collateral effect. The present inventors have found that METH1 and METH2 are potent inhibitors of angiogenesis both in vitro and in vivo. The advantage of METH1 and METH1 is that these inhibitors are normally associated with suppression of physiological angiogenesis; therefore, they offer lack of toxicity and endothelial specificity over other angiogenic inhibitors. Furthermore, METH1 and METH2 present a restricted pattern of expression providing a possible advantage on organ specificity.

Accordingly, the polypeptides of the present invention may be employed to treat cancer. The METH1 and METH2 polypeptides of the present invention can also be used to treat individuals with other disorders that are related to angiogenesis, including abnormal wound healing, inflammation, rheumatoid arthritis, psoriasis, endometrial bleeding disorders, diabetic retinopathy, some forms of macular degeneration, hemangiomas, and arterial-venous malformations.

Thus, the invention provides a method of inhibiting angiogenesis in an individual comprising administering to such an individual a pharmaceutical composition comprising an effective amount of an isolated METH1 polypeptide of the invention, effective to increase the METH1 activity level in such an individual. The invention also provides a method of inhibiting angiogenesis in an individual comprising administering to such an individual a pharmaceutical composition comprising an effective amount of an isolated METH2 polypeptide of the invention, effective to increase the METH2 activity level in such an individual.

METH1 polypeptides which may be used to inhibit angiogenesis in this manner include: METH1 polypeptide encoded by the deposited cDNA including the leader; the mature METH1 polypeptide encoded by the deposited the cDNA minus the leader (i.e., the mature protein); a polypeptide comprising amino acids about 1 to about 950 in SEQ ID NO:2; a polypeptide comprising amino acids about 2 to about 950 in SEQ ID NO:2; a polypeptide comprising amino acids about 29 to about 950 in SEQ ID NO:2; a polypeptide comprising amino acids about 30 to about 950 in SEQ ID NO:2; a polypeptide comprising the metalloprotease domain of METH1, amino acids 235 to 459 in SEQ ID NO:2; a polypeptide comprising the disintegrin domain of METH1, amino acids 460 to 544 in SEQ ID NO:2; a polypeptide comprising the first TSP-like domain of METH1, amino acids 545 to 598 in SEQ ID NO:2; a polypeptide comprising the second TSP-like domain of METH1, amino acids 841 to 894 in SEQ ID NO:2; a polypeptide comprising the third TSP-like domain of METH1, amino acids 895 to 934 in SEQ ID NO:2; a polypeptide comprising amino acids 536 to 613 in SEQ ID NO:2; a polypeptide comprising amino acids 549 to 563 in SEQ ID NO:2; a polypeptide comprising amino acids 542 to 894 of SEQ ID NO:2; and a polypeptide comprising amino acids 801 to 950 of SEQ ID NO:2.

METH2 polypeptides which may be used to inhibit angiogenesis in this manner include: the METH2 polypeptide encoded by the deposited cDNA including the leader; the mature METH2 polypeptide encoded by the deposited the cDNA minus the leader (i.e., the mature protein); a polypeptide comprising amino acids about 1 to about 890 in SEQ ID NO:4; a polypeptide comprising amino acids about 2 to about 890 in SEQ ID NO:4; a polypeptide comprising amino acids about 24 to about 890 in SEQ ID NO:4; a polypeptide comprising amino acids about 112 to about 890 in SEQ ID NO:4; a polypeptide comprising the metalloprotease domain of METH2, amino acids 214 to 439 in SEQ ID NO:4; a polypeptide comprising the disintegrin domain of METH2, amino acids 440 to 529 in SEQ ID NO:4; a polypeptide comprising the first TSP-like domain of METH2, amino acids 530 to 583 in SEQ ID NO:4; a polypeptide comprising the second TSP-like domain of METH2, amino acids 837 to 890 in SEQ ID NO:4; a polypeptide comprising amino acids 280 to 606 in SEQ ID NO:4; and a polypeptide comprising amino acids 529 to 548 in SEQ ID NO:4.

Also included are METH1 or METH2 proteins lacking TSP3; a METH1 or METH2 protein lacking TSP2 and TSP3; a METH1 or METH2 protein lacking TSP3, TSP2, and TSP1; a METH1 or METH2 protein lacking the cysteine-rich domain, TSP1, TSP2, and TSP3; a METH1 or METH2 protein lacking the metalloprotease domain, the cysteine-rich domain, TSP1, TSP2 and TSP3; and a METH1 or METH2 protein lacking the prodomain, the metalloprotease domain, the cysteine-rich domain, TSP1, TSP2, and TSP3. Finally, any combination of these domains are also preferred. For example, the cysteine-rich domain of METH1 may be combined with 1, 2, or 3 TSP domains of METH1. The cysteine-rich domain of METH2 may be combined with 1, 2, or 3 TSP domain of METH12. The metalloprotease domain and the cysteine-rich domain of METH1 may be combined with 1, 2 or 3TSP domains of METH1. The metalloprotease domain and the cysteine-rich domain of METH2 may be combined with 1, 2 or 3 TSP domains of METH2. The prodomain, the metalloprotease domain, and the cysteine-rich domain of METH1 may be combined with 1 ,2 or 3 TSP domains of METH1. The prodomain, the metalloprotease domain, and the cysteine-rich domain of METH2 may be combined with 1, 2 or 3 TSP domains of METH2. The signal sequence, the prodomain, the metalloprotease domain, and the cysteine-rich domain of METH1 may be combined with 1,2, or 3 TSP domains of METH1. The signal sequence, the prodomain, the metalloprotease domain, and the cysteine-rich domain of METH2 may be combined with 1,2, or 3 TSP domains of METH2.

As a general proposition, the total pharmaceutically effective amount of METH1 or METH2 polypeptide administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the polypeptide. If given continuously, the METH1 or METH2 polypeptide is typically administered at a dose rate of about 11 g/kg/hour to about 50 μg/kg/hour, either by 1-4 injections per day or by continuous subcutaneous infusions, for example, using a mini-pump. An intravenous bag solution may also be employed.

Pharmaceutical compositions containing the METH1 or METH2 of the invention may be administered orally, rectally, parenterally, intracistemally, intravaginally, intraperitoneally, topically (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrastemal, subcutaneous and intraarticular injection and infusion.

Gene Therapy Methods

Another aspect of the present invention is to gene therapy methods for treating disorders, diseases and conditions. The gene therapy methods relate to the introduction of nucleic acid (DNA, RNA and antisense DNA or RNA) sequences into an animal to achieve expression of the METH1 and/or METH2 polypeptide of the present invention. This method requires a polynucleotide which codes for a METH1 and/or METH2 polypeptide operatively linked to a promoter and any other genetic elements necessary for the expression of the polypeptide by the target tissue. Such gene therapy and delivery techniques are known in the art, see, for example, WO90/11092, which is herein incorporated by reference.

Thus, for example, cells from a patient may be engineered with a polynucleotide (DNA or RNA) comprising a promoter operably linked to a METH1 and/or METH2 polynucleotide ex vivo, with the engineered cells then being provided to a patient to be treated with the polypeptide. Such methods are well-known in the art. For example, see Belldegrun, A., et al., J. Natl. Cancer Inst. 85:207-216 (1993); Ferrantini, M. et al., Cancer Research 53:1107-1112 (1993); Ferrantini, M. et al., J. Immunology 153: 4604-4615 (1994); Kaido, T., et al., Int. J. Cancer 60: 221-229 (1995); Ogura, H., et al., Cancer Research 50: 5102-5106 (1990); Santodonato, L., et al., Human Gene Therapy 7:1-10 (1996); Santodonato, L., et al., Gene Therapy 4:1246-1255 (1997); and Zhang, J.-F. et al., Cancer Gene Therapy 3: 31-38 (1996)), which are herein incorporated by reference. In one embodiment, the cells which are engineered are arterial cells. The arterial cells may be reintroduced into the patient through direct injection to the artery, the tissues surrounding the artery, or through catheter injection.

As discussed in more detail below, the METH1 and/or METH2 polynucleotide constructs can be delivered by any method that delivers injectable materials to the cells of an animal, such as, injection into the interstitial space of tissues (heart, muscle, skin, lung, liver, and the like). The METH1 and/or METH2 polynucleotide constructs may be delivered in a pharmaceutically acceptable liquid or aqueous carrier.

In one embodiment, the METH1 and/or METH2 polynucleotide is delivered as a naked polynucleotide. The term “naked” polynucleotide, DNA or RNA refers to sequences that are free from any delivery vehicle that acts to assist, promote or facilitate entry into the cell, including viral sequences, viral particles, liposome formulations, lipofectin or precipitating agents and the like. However, the METH1 and/or METH2 polynucleotides can also be delivered in liposome formulations and lipofectin formulations and the like can be prepared by methods well known to those skilled in the art. Such methods are described, for example, in U.S. Pat. Nos. 5,593,972, 5,589,466, and 5,580,859, which are herein incorporated by reference.

The METH1 and/or METH2 polynucleotide vector constructs used in the gene therapy method are preferably constructs that will not integrate into the host genome nor will they contain sequences that allow for replication. Appropriate vectors include pWLNEO, pSV2CAT, pOG44, pXT1 and pSG available from Stratagene; pSVK3, pBPV, pMSG and pSVL available from Pharmacia; and pEFIN5, pcDNA3.1, and pRc/CMV2 available from Invitrogen. Other suitable vectors will be readily apparent to the skilled artisan.

Any strong promoter known to those skilled in the art can be used for driving the expression of METH1 and/or METH2 DNA. Suitable promoters include adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs; the b-actin promoter; and human growth hormone promoters. The promoter also may be the native promoter for METH1 and/or METH2.

Unlike other gene therapy techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The METH1 and/or METH2 polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular, fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They maybe conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked acid sequence injection, an effective dosage amount of DNA or RNA will be in the range of from about 0.05 mg/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration.

The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked METH1 and/or METH2 DNA constructs can be delivered to arteries during angioplasty by the catheter used in the procedure.

The naked polynucleotides are delivered by any method known in the art, including, but not limited to, direct needle injection at the delivery site, intravenous injection, topical administration, catheter infusion, and so-called “gene guns”. These delivery methods are known in the art.

The constructs may also be delivered with delivery vehicles such as viral sequences, viral particles, liposome formulations, lipofectin, precipitating agents, etc. Such methods of delivery are known in the art.

In certain embodiments, the METH1 and/or METH2 polynucleotide constructs are complexed in a liposome preparation. Liposomal preparations for use in the instant invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Felgner et al., Proc. Natl. Acad. Sci. USA (1987) 84:7413-7416, which is herein incorporated by reference); mRNA (Malone et al., Proc. Natl. Acad. Sci. USA (1989) 86:6077-6081, which is herein incorporated by reference); and purified transcription factors (Debs et al., J. Biol. Chem. (1990) 265:10189-10192, which is herein incorporated by reference), in functional form.

Cationic liposomes are readily available. For example, N-[1-2,3-dioleyloxy)propyl]-N,N,N-triethylammonium (DOTMA) liposomes are particularly useful and are available under the trademark Lipofectin, from GIBCO BRL, Grand Island, N.Y. (See, also, Felgner et al., Proc. Natl. Acad. Sci. USA (1987) 84:7413-7416, which is herein incorporated by reference). Other commercially available liposomes include transfectace (DDAB/DOPE) and DOTAP/DOPE (Boehringer).

Other cationic liposomes can be prepared from readily available materials using techniques well known in the art. See, e.g. PCT Publication No. WO 90/11092 (which is herein incorporated by reference) for a description of the synthesis of DOTAP (1,2-bis(oleoyloxy)-3-(trimethylammonio)propane) liposomes. Preparation of DOTMA liposomes is explained in the literature, see, e.g., P. Felgner et al., Proc. Natl. Acad. Sci. USA 84:7413-7417, which is herein incorporated by reference. Similar methods can be used to prepare liposomes from other cationic lipid materials.

Similarly, anionic and neutral liposomes are readily available, such as from Avanti Polar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

For example, commercially dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is circulated at 15EC. Alternatively, negatively charged vesicles can be prepared without sonication to produce multilamellar vesicles or by extrusion through nucleopore membranes to produce unilamellar vesicles of discrete size. Other methods are known and available to those of skill in the art.

The liposomes can comprise multilamellar vesicles (MLVs), small unilamellar vesicles (SUVs), or large unilamellar vesicles LUVs), with SUVs being preferred. The various liposome-nucleic acid complexes are prepared using methods well known in the art. See, e.g., Straubinger et al., Methods of Immunology (1983), 101:512-527, which is herein incorporated by reference. For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments. LUVs are prepared by a number of methods, well known in the art. Commonly used methods include Ca²⁺-EDTA chelation (Papahadjopoulos et al., Biochim. Biophys. Acta (1975) 394:483; Wilson et al., Cell (1979) 17:77); ether injection (Deamer, D. and Bangham, A., Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); detergent dialysis (Enoch, H. and Strittmatter, P., Proc. Natl. Acad. Sci. USA (1979) 76:145); and reverse-phase evaporation (REV) (Fraley et al., J. Biol. Chem. (1980) 255:10431; Szoka, F. and Papahadjopoulos, D., Proc. Natl. Acad. Sci. USA (1978) 75:145; Schaefer-Ridder et al., Science (1982) 215:166), which are herein incorporated by reference.

Generally, the ratio of DNA to liposomes will be from about 10:1 to about 1:10. Preferably, the ration will be from about 5:1 to about 1:5. More preferably, the ratio will be about 3:1 to about 1:3. Still more preferably, the ratio will be about 1:1.

U.S. Pat. No. 5,676,954 (which is herein incorporated by reference) reports on the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and international publication no. WO 94/29469 (which are herein incorporated by reference) provide cationic lipids for use in transfecting DNA into cells and mammals. U.S. Pat. Nos. 5,589,466, 5,693,622, 5,580,859, 5,703,055, and international publication no. WO 94/29469 (which are herein incorporated by reference) provide methods for delivering DNA-cationic lipid complexes to mammals.

In certain embodiments, cells are engineered, ex vivo or in vivo, using a retroviral particle containing RNA which comprises a sequence encoding METH1 and/or METH2. Retroviruses from which the retroviral plasmid vectors may be derived include, but are not limited to, Moloney Murine Leukemia Virus, spleen necrosis virus, Rous sarcoma Virus, Harvey Sarcoma Virus, avian leukosis virus, gibbon ape leukemia virus, human immunodeficiency virus, Myeloproliferative Sarcoma Virus, and mammary tumor virus.

The retroviral plasmid vector is employed to transduce packaging cell lines to form producer cell lines. Examples of packaging cells which may be transfected include, but are not limited to, the PE501, PA317, R-2, R-AM, PA12, T19-14×, VT-19-17-H2, RCRE, RCRIP, GP+E-86, GP+envAml2, and DAN cell lines as described in Miller, Human Gene Therapy 1:5-14 (1990), which is incorporated herein by reference in its entirety. The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include polynucleotide encoding METH1 and/or METH2. Such retroviral vector particles then may be employed, to transduce eukaryotic cells, either in vitro or in vivo. The transduced eukaryotic cells will express METH1 and/or METH2.

In certain other embodiments, cells are engineered, ex vivo or in vivo, with METH1 and/or METH2 polynucleotide contained in an adenovirus vector. Adenovirus can be manipulated such that it encodes and expresses METH1 and/or METH2, and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartz, A. R. et al. (1974) Am. Rev. Respir. Dis. 109:233-238). Finally, adenovirus mediated gene transfer has been demonstrated in a number of instances including transfer of alpha-1-antitrypsin and CFTR to the lungs of cotton rats (Rosenfeld, M. A. et al. (1991) Science 252:431-434; Rosenfeld et al., (1992) Cell 68:143-155). Furthermore, extensive studies to attempt to establish adenovirus as a causative agent in human cancer were uniformly negative (Green, M. et al. (1979) Proc. Natl. Acad. Sci. USA 76:6606).

Suitable adenoviral vectors useful in the present invention are described, for example, in Kozarsky and Wilson, Curr. Opin. Genet. Devel. 3:499-503 (1993); Rosenfeld et al., Cell 68:143-155 (1992); Engelhardt et al., Human Genet. Ther. 4:759-769 (1993); Yang et al., Nature Genet. 7:362-369 (1994); Wilson et al., Nature 365:691-692 (1993); and U.S. Pat. No. 5,652,224, which are herein incorporated by reference. For example, the adenovirus vector Ad2 is useful and can be grown in human 293 cells. These cells contain the E1 region of adenovirus and constitutively express E1 and E1, which complement the defective adenoviruses by providing the products of the genes deleted from the vector. In addition to Ad2, other varieties of adenovirus (e.g., Ad3, Ad5, and Ad7) are also useful in the present invention.

Preferably, the adenoviruses used in the present invention are replication deficient. Replication deficient adenoviruses require the aid of a helper virus and/or packaging cell line to form infectious particles. The resulting virus is capable of infecting cells and can express a polynucleotide of interest which is operably linked to a promoter, but cannot replicate in most cells. Replication deficient adenoviruses may be deleted in one or more of all or a portion of the following genes: E1, E1, E3, E4, E2a, or L1 through L5.

In certain other embodiments, the cells are engineered, ex vivo or in vivo, using an adeno-associated virus (AAV). AAVs are naturally occurring defective viruses that require helper viruses to produce infectious particles (Muzyczka, N., Curr. Topics in Microbiol. Immunol. 158:97 (1992)). It is also one of the few viruses that may integrate its DNA into non-dividing cells. Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377.

For example, an appropriate AAV vector for use in the present invention will include all the sequences necessary for DNA replication, encapsidation, and host-cell integration. The METH1 and/or METH2 polynucleotide construct is inserted into the AAV vector using standard cloning methods, such as those found in Sambrook et al., Molecular Cloning: A Laboratory Manual, Cold Spring Harbor Press (1989). The recombinant AAV vector is then transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the METH1 and/or METH2 polynucleotide construct. These viral particles are then used to transduce eukaryotic cells, either ex vivo or in vivo. The transduced cells will contain the METH1 and/or METH2 polynucleotide construct integrated into its genome, and will express METH1 and/or METH2.

Another method of gene therapy involves operably associating heterologous control regions and endogenous polynucleotide sequences (e.g. encoding METH1 and/or METH2) via homologous recombination (see, e.g., U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989). This method involves the activation of a gene which is present in the target cells, but which is not normally expressed in the cells, or is expressed at a lower level than desired.

Polynucleotide constructs are made, using standard techniques known in the art, which contain the promoter with targeting sequences flanking the promoter. Suitable promoters are described herein. The targeting sequence is sufficiently complementary to an endogenous sequence to permit homologous recombination of the promoter-targeting sequence with the endogenous sequence. The targeting sequence will be sufficiently near the 5′ end of the METH1 and/or METH2 desired endogenous polynucleotide sequence so the promoter will be operably linked to the endogenous sequence upon homologous recombination.

The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter. The amplified promoter and targeting sequences are digested and ligated together.

The promoter-targeting sequence construct is delivered to the cells, either as naked polynucleotide, or in conjunction with transfection-facilitating agents, such as liposomes, viral sequences, viral particles, whole viruses, lipofection, precipitating agents, etc., described in more detail above. The P promoter-targeting sequence can be delivered by any method, included direct needle injection, intravenous injection, topical administration, catheter infusion, particle accelerators, etc. The methods are described in more detail below.

The promoter-targeting sequence construct is taken up by cells. Homologous recombination between the construct and the endogenous sequence takes place, such that an endogenous METH1 and/or METH2 sequence is placed under the control of the promoter. The promoter then drives the expression of the endogenous METH1 and/or METH2 sequence.

The polynucleotides encoding METH1 and/or METH2 may be administered along with other polynucleotides encoding other proteins. Such proteins include, but are not limited to, acidic and basic fibroblast growth factors, VEGF-1, VEGF-2, VEGF-3, VEGF-E, PIGF 1 and 2, epidermal growth factor alpha and beta, platelet-derived endothelial cell growth factor, platelet-derived growth factor alpha and beta, tumor necrosis factor alpha, hepatocyte growth factor, insulin like growth factor, colony stimulating factor, macrophage colony stimulating factor, granulocyte/macrophage colony stimulating factor, and nitric oxide synthase.

Preferably, the polynucleotide encoding METH1 and/or METH2 contains a secretory signal sequence that facilitates secretion of the protein. Typically, the signal sequence is positioned in the coding region of the polynucleotide to be expressed towards or at the 5′ end of the coding region. The signal sequence may be homologous or heterologous to the polynucleotide of interest and may be homologous or heterologous to the cells to be transfected. Additionally, the signal sequence may be chemically synthesized using methods known in the art.

Any mode of administration of any of the above-described polynucleotides constructs can be used so long as the mode results in the expression of one or more molecules in an amount sufficient to provide a therapeutic effect. This includes direct needle injection, systemic injection, catheter infusion, biolistic injectors, particle accelerators (i.e., “gene guns”), gelfoam sponge depots, other commercially available depot materials, osmotic pumps (e.g., Alza minipumps), oral or suppositorial solid (tablet or pill) pharmaceutical formulations, and decanting or topical applications during surgery. For example, direct injection of naked calcium phosphate-precipitated plasmid into rat liver and rat spleen or a protein-coated plasmid into the portal vein has resulted in gene expression of the foreign gene in the rat livers (Kaneda et al., Science 243:375 (1989)).

A preferred method of local administration is by direct injection. Preferably, a recombinant molecule of the present invention complexed with a delivery vehicle is administered by direct injection into or locally within the area of arteries. Administration of a composition locally within the area of arteries refers to injecting the composition centimeters and preferably, millimeters within arteries.

Another method of local administration is to contact a polynucleotide construct of the present invention in or around a surgical wound. For example, a patient can undergo surgery and the polynucleotide construct can be coated on the surface of tissue inside the wound or the construct can be injected into areas of tissue inside the wound.

Therapeutic compositions useful in systemic administration include recombinant molecules of the present invention complexed to a targeted delivery vehicle of the present invention. Suitable delivery vehicles for use with systemic administration comprise liposomes comprising ligands for targeting the vehicle to a particular site.

Preferred methods of systemic administration, include intravenous injection, aerosol, oral and percutaneous (topical) delivery. Intravenous injections can be performed using methods standard in the art. Aerosol delivery can also be performed using methods standard in the art (see, for example, Stribling et al., Proc. Natl. Acad. Sci. USA 189:11277-11281(1992), which is incorporated herein by reference). Oral delivery can be performed by complexing a polynucleotide construct of the present invention to a carrier capable of withstanding degradation by digestive enzymes in the gut of an animal. Examples of such carriers, include plastic capsules or tablets, such as those known in the art. Topical delivery can be performed by mixing a polynucleotide construct of the present invention with a lipophilic reagent (e.g., DMSO) that is capable of passing into the skin.

Determining an effective amount of substance to be delivered can depend upon a number of factors including, for example, the chemical structure and biological activity of the substance, the age and weight of the animal, the precise condition requiring treatment and its severity, and the route of administration. The frequency of treatments depends upon a number of factors, such as the amount of polynucleotide constructs administered per dose, as well as the health and history of the subject. The precise amount, number of doses, and timing of doses will be determined by the attending physician or veterinarian.

Therapeutic compositions of the present invention can be administered to any animal, preferably to mammals and birds. Preferred mammals include humans, dogs, cats, mice, rats, rabbits sheep, cattle, horses and pigs, with humans being particularly preferred.

In a specific embodiment, nucleic acids comprising sequences encoding antibodies or functional derivatives thereof, are administered to treat, inhibit or prevent a disease or disorder associated with aberrant expression and/or activity of a polypeptide of the invention, by way of gene therapy. Gene therapy refers to therapy performed by the administration to a subject of an expressed or expressible nucleic acid. In this embodiment of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 11(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, the compound comprises nucleic acid sequences encoding an antibody, said nucleic acid sequences being part of expression vectors that express the antibody or fragments or chimeric proteins or heavy or light chains thereof in a suitable host. In particular, such nucleic acid sequences have promoters operably linked to the antibody coding region, said promoter being inducible or constitutive, and, optionally, tissue-specific. In another particular embodiment, nucleic acid molecules are used in which the antibody coding sequences and any other desired sequences are flanked by regions that promote homologous recombination at a desired site in the genome, thus providing for intrachromosomal expression of the antibody encoding nucleic acids (Koller and Smithies, Proc. Natl. Acad. Sci. USA 86:8932-8935 (1989); Zijlstra et al., Nature 342:435-438 (1989). In specific embodiments, the expressed antibody molecule is a single chain antibody; alternatively, the nucleic acid sequences include sequences encoding both the heavy and light chains, or fragments thereof, of the antibody.

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/20316; WO93/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contains nucleic acid sequences encoding an antibody of the invention are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a patient. More detail about retroviral vectors can be found in Boesen et al., Biotherapy 6:291-302 (1994), which describes the use of a retroviral vector to deliver the mdr1 gene to hematopoietic stem cells in order to make the stem cells more resistant to chemotherapy. Other references illustrating the use of retroviral vectors in gene therapy are: Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431434 (1991); Rosenfeld et al., Cell 68:143-155 (1992); Mastrangeli et al., J. Clin. Invest. 91:225-234 (1993); PCT Publication WO94/12649; and Wang, et al., Gene Therapy 2:775-783 (1995). In a preferred embodiment, adenovirus vectors are used.

Adeno-associated virus (AAV) has also been proposed for use in gene therapy (Walsh et al., Proc. Soc. Exp. Biol. Med. 204:289-300 (1993); U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcell-mediated gene transfer, spheroplast fusion, etc. Numerous techniques are known in the art for the introduction of foreign genes into cells (see, e.g., Loeffler and Behr, Meth. Enzymol. 217:599-618 (1993); Cohen et al., Meth. Enzymol. 217:618-644 (1993); Cline, Pharmac. Ther. 29:69-92m (1985) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T-lymphocytes, B-lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the patient.

In an embodiment in which recombinant cells are used in gene therapy, nucleic acid sequences encoding an antibody are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g., PCT Publication WO 94/08598; Stemple and Anderson, Cell 71:973-985 (1992); Rheinwald, Meth. Cell Bio. 21A:229 (1980); and Pittelkow and Scott, Mayo Clinic Proc. 61:771 (1986)).

In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

The compounds or pharmaceutical compositions of the invention are preferably tested in vitro, and then in vivo for the desired therapeutic or prophylactic activity, prior to use in humans. For example, in vitro assays to demonstrate the therapeutic or prophylactic utility of a compound or pharmaceutical composition include, the effect of a compound on a cell line or a patient tissue sample. The effect of the compound or composition on the cell line and/or tissue sample can be determined utilizing techniques known to those of skill in the art including, but not limited to, rosette formation assays and cell lysis assays. In accordance with the invention, in vitro assays which can be used to determine whether administration of a specific compound is indicated, include in vitro cell culture assays in which a patient tissue sample is grown in culture, and exposed to or otherwise administered a compound, and the effect of such compound upon the tissue sample is observed.

Therapeutic Prophylactic Administration and Composition

The invention provides methods of treatment, inhibition and prophylaxis by administration to a subject of an effective amount of a compound or pharmaceutical composition of the invention, preferably an antibody of the invention. In a preferred aspect, the compound is substantially purified (e.g., substantially free from substances that limit its effect or produce undesired side-effects). The subject is preferably an animal, including but not limited to animals such as cows, pigs, horses, chickens, cats, dogs, etc., and is preferably a mammal, and most preferably human.

Formulations and methods of administration that can be employed when the compound comprises a nucleic acid or an immunoglobulin are described above; additional appropriate formulations and routes of administration can be selected from among those described herein below.

Various delivery systems are known and can be used to administer a compound of the invention, e.g., encapsulation in liposomes, microparticles, microcapsules, recombinant cells capable of expressing the compound, receptor-mediated endocytosis (see, e.g., Wu and Wu, J. Biol. Chem. 262:4429-4432 (1987)), construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the invention, care must be taken to use materials to which the protein does not absorb.

In another embodiment, the compound or composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp.317-327; see generally ibid.) In yet another embodiment, the compound or composition can be delivered in a controlled release system. In one embodiment, a pump may be used (see Langer, supra; Sefton, CRC Crit. Ref Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J., Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled release system can be placed in proximity of the therapeutic target, i.e., the brain, thus requiring only a fraction of the systemic dose (see, e.g., Goodson, in Medical Applications of Controlled Release, supra, vol. 2, pp. 115-138 (1984)).

Other controlled release systems are discussed in the review by Langer (Science 249:1527-1533 (1990)).

In a specific embodiment where the compound of the invention is a nucleic acid encoding a protein, the nucleic acid can be administered in vivo to promote expression of its encoded protein, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., Proc. Natl. Acad. Sci. USA 88:1864-1868 (1991)), etc. Alternatively, a nucleic acid can be introduced intracellularly and incorporated within host cell DNA for expression, by homologous recombination.

The present invention also provides pharmaceutical compositions. Such compositions comprise a therapeutically effective amount of a compound, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the compound, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of the compound of the invention which will be effective in the treatment, inhibition and prevention of a disease or disorder associated with aberrant expression and/or activity of a polypeptide of the invention can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems.

For antibodies, the dosage administered to a patient is typically 0.1 mg/kg to 100 mg/kg of the patient's body weight. Preferably, the dosage administered to a patient is between 0.1 mg/kg and 20 mg/kg of the patient's body weight, more preferably 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention may be reduced by enhancing uptake and tissue penetration (e.g., into the brain) of the antibodies by modifications such as, for example, lipidation.

The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

Chromosome Assays

The nucleic acid molecules of the present invention are also valuable for chromosome identification. The sequence is specifically targeted to and can hybridize with a particular location on an individual human chromosome. The mapping of DNAs to chromosomes according to the present invention is an important first step in correlating those sequences with genes associated with disease.

In certain preferred embodiments in this regard, the cDNA herein disclosed is used to clone genomic DNA of a METH1 or METH2 protein gene. This can be accomplished using a variety of well known techniques and libraries, which generally are available commercially. The genomic DNA then is used for in situ chromosome mapping using well known techniques for this purpose.

In addition, in some cases, sequences can be mapped to chromosomes by preparing PCR primers (preferably 15-25 bp) from the cDNA. Computer analysis of the 3′ untranslated region of the gene is used to rapidly select primers that do not span more than one exon in the genomic DNA, thus complicating the amplification process. These primers are then used for PCR screening of somatic cell hybrids containing individual human chromosomes.

Fluorescence in situ hybridization (“FISH”) of a cDNA clone to a metaphase chromosomal spread can be used to provide a precise chromosomal location in one step. This technique can be used with probes from the cDNA as short as 50 or 60 bp. For a review of this technique, see Verma et al., Human Chromosomes: A Manual Of Basic Techniques, Pergamon Press, New York (1988).

Other mapping strategies that can similarly be used to map to its chromosome include radiation hybrid mapping, prescreening with labeled flow-sorted chromosomes and preselection by hybridization to construct chromosome specific-cDNA libraries. Radiation hybrid (RH) mapping relies upon fragmentation of human chromosomes with X-rays, and retention of these random fragments in Hamster A23 host cells. The DNAs for RH mapping are supplied by Research Genetics (USA). Oligo pairs are designed from EST sequences that will amplify products of between 80 bp and 300 bp. The PCRs are performed on 93 human/hamster hybrid DNAs and the results compared with a framework map (http://www-genome.wi.mit.edu/cgi-bin/contig/rhmapper.pl; Gyapay et al., Human Molecular Genetics 5:39-346 (1996)). RH mapping provides greater precision than FISH and indicates clusters of genes as well as disease locus/gene correlations.

Once a sequence has been mapped to a precise chromosomal location, the physical position of the sequence on the chromosome can be correlated with genetic map data. Such data are found, for example, in V. McKusick, Mendelian Inheritance In Man, available on-line through Johns Hopkins University, Welch Medical Library. The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

The METH1 gene maps between STS markers D21S1435 and D21S1442 which translates as 21q21. This is a similar chromosomal location to amyloid precursor protein (APP). APP and METH1 are approximately 3 million bases apart which is not a massive distance in human genomics. The chromosomal location includes important genes such as enterokinases (enzymes that activate trypsinogen by converting it to trypsin) and genes responsible for Alzheimer's disease.

The METH1 gene can be mapped to 21q21 using the following oligos for radiation hybrid mapping:

| | | |
|---|---|---|
| 5′primer: | ACTGTGTGTGATCCGAG | (SEQ ID NO:126) |
| 3′primer: | GTTGGAAAGCATTGACG | (SEQ ID NO:127) |

Kits

The present invention provides kits that can be used in the above methods. In one embodiment, a kit comprises an antibody of the invention, preferably a purified antibody, in one or more containers. In a specific embodiment, the kits of the present invention contain a substantially isolated polypeptide comprising an epitope which is specifically immunoreactive with an antibody included in the kit. Preferably, the kits of the present invention further comprise a control antibody which does not react with the polypeptide of interest. In another specific embodiment, the kits of the present invention contain a means for detecting the binding of an antibody to a polypeptide of interest (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate).

In another specific embodiment of the present invention, the kit is a diagnostic kit for use in screening serum containing antibodies specific against proliferative and/or cancerous polynucleotides and polypeptides. Such a kit may include a control antibody that does not react with the polypeptide of interest. Such a kit may include a substantially isolated polypeptide antigen comprising an epitope which is specifically immunoreactive with at least one anti-polypeptide antigen antibody. Further, such a kit includes means for detecting the binding of said antibody to the antigen (e.g., the antibody may be conjugated to a fluorescent compound such as fluorescein or rhodamine which can be detected by flow cytometry). In specific embodiments, the kit may include a recombinantly produced or chemically synthesized polypeptide antigen. The polypeptide antigen of the kit may also be attached to a solid support.

In a more specific embodiment the detecting means of the above-described kit includes a solid support to which said polypeptide antigen is attached. Such a kit may also include a non-attached reporter-labeled anti-human antibody. In this embodiment, binding of the antibody to the polypeptide antigen can be detected by binding of the said reporter-labeled antibody.

In an additional embodiment, the invention includes a diagnostic kit for use in screening serum containing antigens of the polypeptide of the invention. The diagnostic kit includes a substantially isolated antibody specifically immunoreactive with polypeptide or polynucleotide antigens, and means for detecting the binding of the polynucleotide or polypeptide antigen to the antibody. In one embodiment, the antibody is attached to a solid support. In a specific embodiment, the antibody may be a monoclonal antibody. The detecting means of the kit may include a second, labeled monoclonal antibody. Alternatively, or in addition, the detecting means may include a labeled, competing antigen.

In one diagnostic configuration, test serum is reacted with a solid phase reagent having a surface-bound antigen obtained by the methods of the present invention. After binding with specific antigen antibody to the reagent and removing unbound serum components by washing, the reagent is reacted with reporter-labeled anti-human antibody to bind reporter to the reagent in proportion to the amount of bound anti-antigen antibody on the solid support. The reagent is again washed to remove unbound labeled antibody, and the amount of reporter associated with the reagent is determined. Typically, the reporter is an enzyme which is detected by incubating the solid phase in the presence of a suitable fluorometric, luminescent or colorimetric substrate (Sigma, St. Louis, Mo.).

The solid surface reagent in the above assay is prepared by known techniques for attaching protein material to solid support material, such as polymeric beads, dip sticks, 96-well plate or filter material. These attachment methods generally include non-specific adsorption of the protein to the support or covalent attachment of the protein, typically through a free amine group, to a chemically reactive group on the solid support, such as an activated carboxyl, hydroxyl, or aldehyde group. Alternatively, streptavidin coated plates can be used in conjunction with biotinylated antigen(s).

Thus, the invention provides an assay system or kit for carrying out this diagnostic method. The kit generally includes a support with surface-bound recombinant antigens, and a reporter-labeled anti-human antibody for detecting surface-bound anti-antigen antibody.

Having generally described the invention, the same will be more readily understood by reference to the following examples, which are provided by way of illustration and are not intended as limiting.

### EXAMPLES

### Example 1

### Identification and Cloning of METH1 and METH2

To search for novel genes with TSP-like domains, a large human cDNA database consisting of approximately 900,000 expressed sequence tags (ESTs) was screened for sequences homologous to the second type I repeat of TSP1. Several ESTs were predicted to encode proteins with TSP-like domains. Two cDNA clones originated from human heart and lung libraries were further sequenced and chosen for functional analysis.

The amino-terminal end of METH1 was obtained using 5′ rapid amplification of cDNA ends (RACE) PCR technique (Marathon cDNA amplification kit, Clontech) according to manufacturer instructions. The amino-terminal end of METH2 was obtained partially through 5′RACE PCR and later confirmed and completed by genomic screening. For the genomic screen, BAC clones (Genome Systems) were initially identified by PCR. Positive BAC clones containing 150-200 bp of sequence were subsequently subcloned into pGEM vector as small fragments and sequenced.

Analysis and comparison of the deduced amino acid sequence with the GenBank, EMBL and SwissProt databases suggested that these genes belong to a new family of metalloproteases with homology to the reprolysin family in their NH2-terminal end and with several TSP-like motifs in the COOH-terminal end. These cDNAs were named METH1 and METH2; ME, for metalloprotease and TH, for thrombospondin. The mouse homologue of METH1 was identified and named ADAMTS1 (Kuno, K., et al., J. Biol. Chem. 272:556-562 (1997)). Direct comparison of the human and mouse sequences revealed a high level of conservation (83.4% amino acid identity). Thus far no homologues for METH2 have been identified.

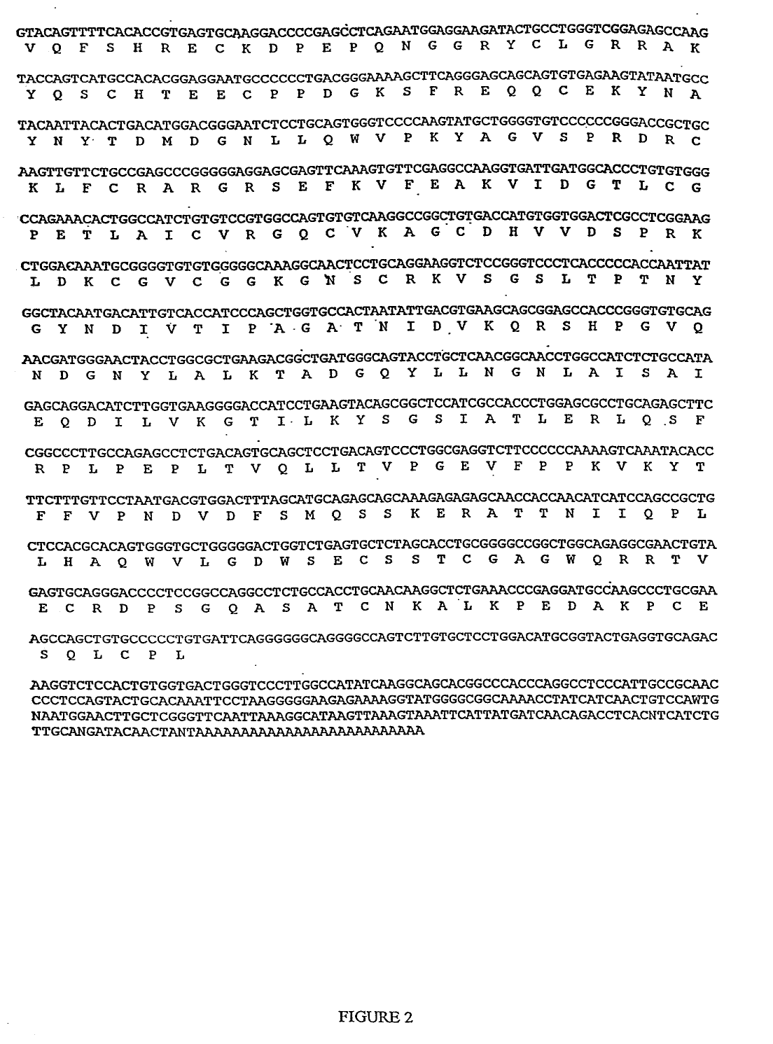

Interestingly, a recently identified protein named pNPI (procollagen 1 N-proteinase; (Colidge, A., et al., Proc. Natl. Acad. Sci. USA 94:2374-2379 (1997)) showed a striking sequence and structural similarity to METH1 and METH2 (FIG. 3). As the novel proteins described here, pNPI also contains metalloproteinase (reprolysin subfamily) and TSP domains at the carboxy-terminal end. Although the sequence for pNPI is of bovine origin, sequence alignment revealed identical structural features. The amino acid similarity between METH1 and METH2 is 51.7%, and between METH1 or METH2 and pNPI the homology is lesser 33.9% and 36.3%, respectively.

Sequence analysis showed that the ORF of METH1 and METH2 coded for proteins of 950 and 890 amino acids, respectively. In all three proteins, the NH2 terminal end contains a putative signal peptide followed by another putative transmembrane domain around amino acid 300, deduced from the hydrophilicity plots. It is not clear whether these proteins are bound to the membrane. However, given preliminary data, it is more likely that this second transmembrane domain will consist of a hydrophobic pocket and that METH1, METH2 and pNPI are in fact secreted proteins. The NH2-terminal end past the signal peptide has homology to the superfamily of zinc metalloproteases and can be subdivided in a prodomain, a metalloprotease domain, and a cysteine-rich region.

The double underlined sequence in METH1 (amino acids 232-235) and METH2 (amino acids 211-214) in FIG. 3 localized at the boundary between the prodomain and the metalloprotease domain, are potential cleavage sites for mammalian subtilisins, such as furins (Barr, 1991). Proteolytical processing occurs in SVMPs to yield soluble metalloproteases and disintegrins (Bjarnason, J. B. & Fox, J. W., Methods Enzymol. 248:345-368 (1995)) and has also been detected in some ADAMs (reviewed by Wolsberg, T. G. & White, J. M., Developmental Biology 180:389401 (1996)). Proteolytical processing occurs in both METH1 and 2 (see below). Additionally, both METH1 and METH2 present a Zn²⁺-binding site (dotted line in FIG. 3) that is presumed to be catalytically active due to the conservation of certain functionally important amino acids (Rawlings, N. D. & Barrett, A. J., Methods Enzymol. 248:183-228 (1995)) suggesting that these proteins may be active proteases.

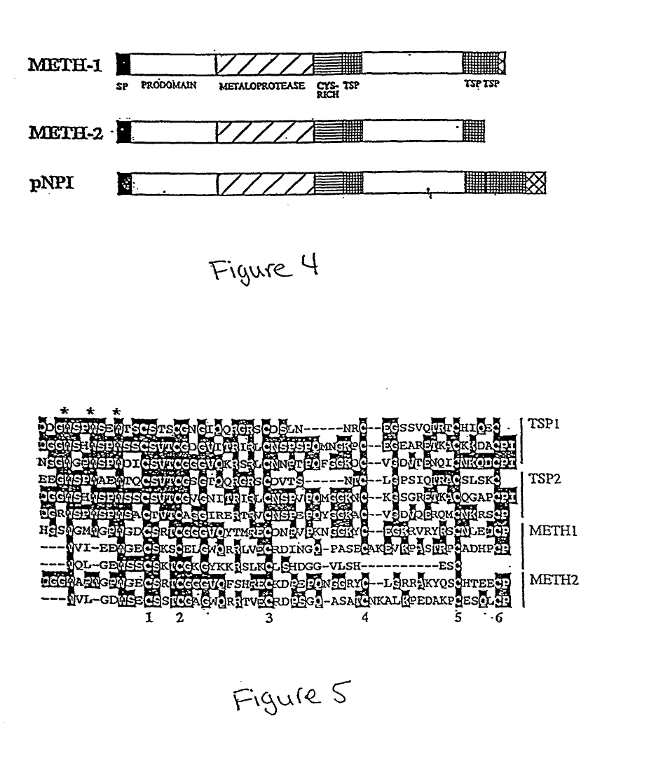

Following the metalloprotease domain, there is a cysteine-rich region which contains two putative disintegrin loops (Wolsberg, T. G. & White, J. M., Developmental Biology 180:389401 (1996)) (marked by arrows in FIG. 3). Disintegrin domains are found within the superfamily of metalloproteases in snake venom metalloproteases (SVMPs) and ADAMs (mammalian proteins containing a disintegrin and a metalloprotease domain) and have a possible function inhibiting binding of integrins to their ligands in SVMPs. Conversely, the ADAM-disintegrin-like domain, as part of membrane anchored proteins, may promote rather than disrupt, cell-cell interactions (Wolsberg, T. G. & White, J. M., Developmental Biology 180:389-401 (1996)). The TSP-like domains are located in the COOH-half of METH1 and METH2 proteins. METH1 contains two conserved TSP domains separated by a spacer region with unknown function, and a subdomain with less homology, and only 5 cysteines, following the second anti-angiogenic region. METH2 contains two TSP domains separated by the spacer region. The alignment of the TSP-like domains of METH1 and METH2 with those of TSP1 and TSP2 are shown in FIG. 5. The homology varies between 19.2% to 52% amino acid similarity among all the TSP repeats. The cysteines, numbered 1 to 6, and the tryptophans, labeled by asterisks, are highly conserved.

Southern blot of human genomic DNA revealed the presence of METH1 and METH2 in the genome. METH1 and METH2 probes revealed bands of different size suggesting that they are transcribed from different genes.

The consensus sequence for the type I repeats includes 16 residues with 6 perfectly conserved cysteines. Typically it begins with the sequence motif WSXWS (SEQ ID NO:82) that has also been shown to bind to heparin (Guo, N., et al., J. Biol. Chem. 267:19349-19355 (1992)). The affinity of this region to heparin has been proposed to the part of the anti-angiogenic activity of TSP-1 (Guo, N., et al., J. Peptide Res. 49 (1997)). Among the five members of the TSP family of proteins, only TSP-1 and TSP-2 inhibit angiogenesis and contain the type I repeats (Tolsma, S. S., et al., J. Cell. Biol. 122:497-511 (1993); Kyriakides, T. R., et al., J. Cell Biol. 140:419-430 (1998)). The type I or properdin repeats were probably added to the precursor of TSP1 and 2 by exon shuffling between 500 and 900 million years ago (Adams, J., et al., The Thrombospondin Gene Family, I Ed. Molecular Biology Intelligence Unit (Springer, Ed.), R. G. Landes Company, Germany (1995)). It is likely that the acquisition of this domain provided the precursor of TSP1 and TSP2 with functions, such as regulation of new vessel formation. More recently, BAI-1 (brain angiogenic inhibitor-1), a protein isolated from a brain library for its ability to be regulated by p53, has also been shown to contain the type I repeat of TSP-1 and to provide anti-angiogenic potential to this molecule (Nishimori, H., et al., Oncogene 15:2145-2150 (1997)). Nevertheless, it appears that additional sequences or context are also important, since other proteins containing the type I repeats appear not to have clear or more established anti-angiogenic properties such as: properdin, F-spondin, and other members of the complement family.

Because of the presence of TSP-repeats in METH1 and METH2, along with their anti-angiogenic properties, these proteins were originally considered members of the TSP superfamily. Nevertheless, they have no additional homology to other TSPs, and in fact, the similarity to TSP1 and TSP2 is restricted to the type I repeats. Furthermore, the proteins also have strong sequence and structural homology to members of the ADAM family. These features led Kuno and colleagues to name ADAMTS to the mouse homolog of METH1 (Kuno, K., et al., J. Biol. Chem. 272:556-562 (1997)). The recent identification of pNPI and its striking sequence homology to the proteins here described, prompt all these three proteins to be grouped in a subfamily named metallospondins. At this point, it is not clear whether pNIP has anti-angiogenic properties or whether METH1 and/or METH2 participate in the cleavage of the amino terminal pro-peptide of α1(I) procollagen.

### Example 2

### Northern and Southern Blot Analysis

Total RNA was purified from cells by guanidinium-isothiocyanate extraction, as previously described (Chomczynski, P. & Sacchi, N., Anal. Biochem. 162:156-159 (1987)) Poly(A)+ RNA was extracted using a Boehringer Mannheim (BMB, Indianapolis, Ind.) kit according to the manufacturer conditions. Other poly(A)+ RNA blots were purchased from Clontech (Palo Alto, Calif.). Pre-hybridization was performed in a solution containing: 50% formamide, 6× SSPE, 1× Denhardt's solution, 0.1% SDS and 100 μg/ml of heat denatured salmon sperm DNA for 12-18 h at 42° C. Hybridization with labeled cDNA probes proceeded in the same solution at 42° C. for 12-18 h. TSP1 and METH1 probes corresponded to the entire human cDNAs. The METH2 probe corresponded to a KpnI-EcoRI fragment from the human cDNA. A 1.3 Kb PstI fragment of the glyceraldehyde-3-phosphate-dehydrogenase (GPDH) was used to normalize for loading and transfer efficiency. Membranes were exposed to Kodak Biomax MS film (Kodak, New Haven, Conn.).

For Southern blots, human genomic DNA, purchased from Promega (Madison, Wis.), was heated at 65° C. for 10 min and digested with EcoRI and PstI overnight at 37° C. 5 μg of digested DNA was separated in a 1% agarose gel, transferred to a nytran membrane and cross-linked by ultraviolet light. cDNA probes, as well as, prehybridization and hybridization conditions were identical to those described for Northern blots. Blots were washed with high stringency (0.2× SSC, 0.2% SDS at 50° C.).

The expression patterns of METH1 and METH2 were examined in both adult and embryonic tissues. Northern blot analysis was performed under high-stringency conditions with blots that included poly(A)+ RNA from human tissues. METH1 and METH2 transcripts revealed a single band of 4.6 and 3.7 Kb, respectively. Abundant METH1 mRNA expression was observed in adrenal, heart, placenta, followed by skeletal muscle, thyroid and stomach. From the embryonic tissues analyzed, kidney showed the highest expression of METH1 mRNA. Nevertheless, weaker expression of METH1 mRNA was seen in all tissues analyzed. Distribution of METH2 mRNA was more restricted and weaker than that of METH1. The highest expression was seen in lung, both embryonic and adult. Interestingly, METH1 and METH2 expression do not appear to overlap. In combination, the structural similarities and their pattern of expression suggest functional redundancy yet different transcriptional regulation. The expression levels of TSP1 transcripts in the same blots were also analyzed, for purpose of comparison. TSP1 mRNA highest expression was seen in the adult placenta and in all embryonic tissues analyzed. In contrast to METH1 and METH2 we observed constant levels of TSP1 transcript in all the other tissues examined.

The cell type distribution was also studied by Northern blot analysis of poly(A)+ RNA. METH1 mRNA was detectable, at low levels, in dermal fibroblasts, vascular smooth muscle, endometrial stromal cells, and in two cancer cell lines, HeLa and G631, an adenocarcinoma and a melanoma, respectively. METH2 mRNA was detected only on SW480, a colon carcinoma cell line, but no expression was seen in any other of the cell lines or primary strains analyzed.

The possibility that groups of angiogenic and anti-angiogenic factors regulate vascular network formation in specific organs has been a frequently discussed hypothesis likely to be true, yet unproven. The expression patterns of METH1 and METH2, which are clearly distinct and almost non-overlapping, were puzzling, at least with concern to overall levels. TSP1 and TSP2 also share identical structure, high level of amino acid similarity, yet their pattern of expression differs significantly (Iruela-Arispe, M. L., Dev. Dyn. 197:40-56 (1993)). The differences are likely based on dissimilar cis-acting elements in their promoters and different regulatory mechanisms, as previously suggested. Although the promoters for METH1 and 2 have not been characterized, it is likely that they provide unique features for the regulation of each gene. Nevertheless, the possibility that one motif, the anti-angiogenic/type I repeat, with demonstrated anti-angiogenic properties is present in several proteins with different tissue specificities is appealing. Alternatively, the small differences in sequence between closely related members of the same family could possess significance that goes beyond functional redundancy. In the case of TSP 1 and TSP2, aside from the striking structural similarities and perhaps having functionally common anti-angiogenic properties, TSP1 and TSP2 also appear to display functions of their own and not likely shared by their similar relative. This became evident with the outcome of the two knock-outs for these genes. TSP1 null animals exhibited primarily lung disorders (Lawler, J., et al., J. Clin. Invest. 101:982-992 (1998)) and secondarily vascular abnormalities, but only under specific pathological settings or on a restricted set of organs. In contrast TSP2 knock-out mice exhibited unpredicted collagen assembly anomalies, with carry-on consequences to the skin, tendons, and bone (Kyriakides, T. R., et al., J. Cell Biol. 140:419-430 (1998)). In addition, these animals also appear to have overall increase in capillary density in the dermis. It is not understood how the resemblance between the newly described members of the metallospondin family translate functionally. Clearly, pNIP has been shown to display active proteolytic activity by cleaving the N-terminus of type I procollagen (Colidge, A., et al., Proc. Natl. Acad. Sci. USA 94:2374-2379 (1997)).

A second region of functional interest corresponds to the disintegrin domain. This domain has been more fully characterized in related members of the snake venom metalloproteases that have been shown to bind to αIIbβ3 and inhibit platelet interaction blocking coagulation (Pfaff, M., et al., Cell Adhes Commun. 2:491-501 (1994); Usami, Y., et al., Biochem. Biophys. Res. Commun. 201:331-339 (1994)). The disintegrin motif consists of a thirteen to fifteen domain which frequently contain an RGD or a negatively charged residue at the position of the aspartic acid. The RGD, or equivalent, binds to integrins and serve as antagonist or signaling ligands (Wolsberg, T. G. & White, J. M., Developmental Biology 180:389401 (1996)). METH2, but not METH1, has an RGD sequence located amino-terminal to the disintegrin domain. In addition, both molecules present relatively high, but not perfect, degree of conservation of cysteines within the disintegrin motif. This appears to display an important role in the tertiary structure of this region and its ability to interact with integrins. In addition, some of these domains have been shown to act as functional adhesion molecules, particularly those with transmembrane regions (Wolsberg, T. G. & White, J. M., Developmental Biology 180:389-401 (1996)). It is unlikely that this will be the case for METH1 and METH2, since both these proteins appear to be secreted.

### Example 3

### Expression and Purification of Recombinant Proteins

Recombinant constructs for expression of His-tagged fusion proteins were generated for expression in bacteria. METH1 nt 605-1839 (from ATG) was amplified by polymerase chain reaction using primers containing BamHI and PstI sites and subcloned into the pRSET vector (Invitrogen, Carlsbad, Calif.). The construct was sequenced to verify frame and sequence fidelity and were then transformed into BL21; DE3 E. Coli strain (Stratagene Cloning Systems, La Jolla, Calif.). Purification was performed by affinity chromatography on Ni-NTA columns. Recombinant protein was eluted with 500 mM imidazole in PBS. Fractions containing recombinant protein were dialyzed against phenol-red free DMEM and used to generate antisera.

Antisera was generated by intramuscular injection of a 1:1 mixture of recombinant protein (500 μg/ml) and Freud's adjuvant. Eight animals, including five guinea pigs and three rabbits were injected every 15 days for three cycles. After the third injection, serum was evaluated for presence of anti-METH1 antibodies, only two of the guinea pigs showed significant titers. The antibodies recognized recombinant protein on Western blots, were able to immunoprecipitate METH1 protein from cell extracts and recognize the protein by immunocytochemistry. Pre-immune sera was always included as control. One of the guinea pig antibodies was also able to recognize METH2.

For mammalian expression, full-length METH1 and METH2 cDNA were cloned into pcDNA3.1 expression vector (Invitrogen). The vector is under the regulatory control of the CMV promoter. Cloning was performed so that constructs contained their own termination codons.

Recombinant protein was obtained by transient transfection of the expression vectors in 293T cells using standard calcium phosphate precipitation. Upon transfection, cells were incubated for 6 to 16 h in serum-containing media and then switched to serum-free media for 36 h for accumulation of recombinant protein. As control, pcDNA3.1 vector alone was transiently transfected in parallel plates. Purification of the protein included 30% ammonium sulfate precipitation followed by dialysis on HS buffer (DB=10 mM HEPES, 150 mM NaCl, 1 mM CaCl₂ and 1 mM MgSO₄). Samples were then subjected to heparin-affinity chromatography. Elution from heparin columns was achieved with HS buffer containing 550 mM NaCl. Fractions were then loaded on 5-30% sucrose gradients and spun at 48K. Separation on sucrose gradients was assessed by Western blotting and purity was determined by Commmassie blue and silver nitrate staining.

Generation of recombinant protein was initially done in bacteria. A METH1 expression vector was generated containing an amino terminal His Tag to aid on the purification. The resulting protein coded for all METH1 translated sequence except the prodomain. Affinity chromatography on Ni⁺⁺-beads showed an unique band of 68 kD. Isolation and purification was always performed under denatured conditions and attempts to refold the protein met with little success, probably due to a significant number of intramolecular disulfide bonds associated with the large number of cysteines. Nonetheless, the protein was used to generate antibodies. From eight animals injected, only two were able to mount an immune response and generate specific antibodies, possibly due to the high conservation across species. Both antibodies recognized recombinant METH1 protein before and after purification on Ni++columns. The antibody was also used to evaluate expression of the protein on Western blots of cell lysates. A single band of approximately 105-110 kD was detected in stromal fibroblasts and smooth muscle cells.

To test the hypothesis that METH1 and METH2 could function as regulators of angiogenesis, recombinant full length protein was generated in mammalian cells. Evaluation of correct reading frame and molecular weight was initially tested by in vitro translation. Translation of the METH-1 open reading frame revealed a 110 kD protein, slightly higher than the size predicted by translation of the cDNA sequence. As previously indicated, there are two putative glycosylation sites, the higher size of the protein is likely due to addition of sugar residues. Similarly, METH2 was also slightly higher than its predicted size, showing a 98 kD protein.

Recombinant proteins were isolated from 293T supernatants under native conditions to preserve secondary structure. From analysis of the deduced amino acid sequence and published information on the murine homolog, ADAMTS, it was predicted that both proteins could bind to heparin and used affinity chromatography for purification. Both cell layer and conditioned media of 293T cells transfected with METH1, METH2 and vector control were used for purification. The molecular weight of METH1 and 2 were similar to those from the reticulocyte lysate. As predicted, both proteins are secreted. Interestingly, the media contains both full length (110 kD) and two processed forms of 85 and 67 kD for METH1, and 79 and 64 kD for METH2. The 85 and 79 kD molecular weights agree with the predicted size for both proteins after cleavage at the consensus sublisin site. However, a second processing event must take place to generate the most abundant fragments observed at 67 and 64 kD respectively. These forms are stable after purification even in the absence of proteinase inhibitors. For purification, proteins were initially concentrated by ammonium sulfate precipitation, followed by dialysis. The resulting protein suspension was then subjected to heparin-sepharose columns. Recombinant METH1 and METH2 were eluted with washing buffer containing 550 mM NaCl. Fractions contained both pro-METH1, as well as the processed forms. Because it was unclear whether processing was relevant for function of the proteins, both forms were separated on sucrose gradients. Both full-length and processed forms were used in angiogenesis assays.

Recombinant constructs for expression of truncated fusion proteins were as follows: (1) pRSET-METH1-Type I: METH1 nt 1605-1839 (from the start codon) was amplified by polymerase chain reaction using the following primers: 5′-GCA TTT TGG ATC CGC CTT TTC ATG-3′ (SEQ ID NO:78) and 5′-GTT GTG TGC TGC AGA TTG TTC C-3′ (SEQ ID NO:79). The amplified fragment was then subcloned into the BamHI and PstI sites of the pRSET vector; (2) pGEX-METH1—TSP was generated by ligating the BamHI-EcoRI fragment from the pRSET-METH1—TSP into the SmaI site of the pGEX-5× vector (Pharmacia Biotech Inc., Piscataway, N.J.) by blunt-end ligation; (3) pGEX-1.0-METH2: the fragment nt 838-1818 of METH2 cDNA (from the start codon) was ligated into BamHI-EcoRI sites of pGEM-2TK. The METH2 fragment was amplified by PCR using the following primers: 5′-GAAAAATGGGGATCCGAGGTG-3′ (SEQ ID NO:80) and 5′-GCAGGAGAATTCCGTCCATG-3′ (SEQ ID NO:81) to generate BamHI and EcoRI restriction sites; (4) pGEX-METH2—TSP: a 0.5 Kb XmaI-EcoRI fragment isolated from pGEX-1.0-METH2 was subcloned into the XmaI and EcoRI sites of pGEX-2TK vector. All constructs were sequenced to verify sequence fidelity and correct open reading frame.

The recombinant proteins were named 6H-METH1, the recombinant protein expressed with the plasmid pRSET-METH1—TSP, GST-METH1, the protein expressed with the plasmid pGEX-METH1—TSP and GST-METH2, the protein expressed with the plasmid pGEX-METH2—TSP.

Expression plasmids were transformed into BL21:DE3 E. coli strain (Stratagene Cloning Systems, La Jolla, Calif.) and fusion proteins were induced following manufacturer recommendations. Briefly, induced bacteria pellets were resuspended in PBS and sonicated on ice for 1 min. The suspension was, subsequently, incubated at RT for 20 min in the presence of 1% triton X-100 and centrifuged at 4° C. Histidine tagged fusion proteins were then purified on Ni-NTA beads (Qiagen, Chatsworth, Calif.) by incubating 20 ml of supernatant with 1 ml of beads (50% slurry) for 2 h at 4° C. The suspension was transferred into a column and washed with 10 columns volume of PBS containing 10 mM imidazole, followed by 50nM imidazole and finally 100 mM imidazole. The protein was eluted with 500 mM imidazole in PBS. Fractions containing the recombinant protein were dialyzed against phenol-red free DMEM. Samples were centrifuged for 30 min at 4° C., part of the protein was not soluble and was lost during centrifugation. The supernatant was stored at −70° C. and used for proliferation, cornea pocket and chorioallantoic membrane (CAM) assays.

For purification of GST-fusion proteins, the extract was cleared by centrifugation and applied to a GST-affinity column (Pharmacia). The column was washed with PBS-1% triton X-100 in the presence of 0.1 mM reduced glutathione and, subsequently, with the same buffer in the presence of 0.5 mM reduced glutathione. Fusion proteins were eluted with 10 mM reduced glutathione in 50 mM Tris-HCl, pH 7.5. Fractions containing the protein were dialyzed against DMEM, stored at −70° C. and used for proliferation, cornea pocket and chorioallantoic membrane (CAM) assays.

Integrity and purity of recombinant proteins was analyzed in 12.5% or 15% acrylamide gels stained with Coomassie blue.

A recombinant GST fusion protein containing the first two type I repeats of TSP was also dialyzed against DMEM before used in functional assays. Intact TSP1 was purified from platelets as previously described (Roberts, D. D., et al., J. Tissue Cult. Methods 16:217-222 (1994)).

To test the hypothesis that METH1 and METH2 TSP domains could function as regulators of angiogenesis recombinant fusion proteins were generated in bacteria. The constructs included the first TSP domain of METH1 or METH2. This domain is the most conserved, 52% amino acid similarity with the second type I repeat of TSP1, (this domain contains a putative binding site for CD36). All recombinant proteins were isolated under native conditions to preserve their secondary structure as much as possible. 6H-METH1 and GST-METH1 contained the first TSP-like domain of METH1 fused to a histidine tag or a GST, respectively. METH1 recombinant protein was made with two different tags because of purification and structural advantages. The differences in size are due to the size of the tag, 6 KDa the histidine and 27 KDa the GST. GST-METH2 contained the first TSP domain of METH2 also fused to a GST. A fragment corresponding to the last two type I repeats of TSP1, also fused to a GST, and intact TSP1 purified from platelets were used as positive controls. In addition, GST alone was included in all experiments as negative control.

### Example 4

### TSP Domains in METH1 and METH2 Disrupt Angiogenesis in vivo

Cornea Pocket Assay

Swiss Webster females and males, were purchased from Charles River (Boston, Mass.) and used between 8-10 weeks-old for implantation of the pellets. Cornea pockets were performed as described by Kenyon and colleagues (Kenyon, B. M., et al., Invest. Ophthalmol. Vis. Sci. 37:1625-1632 (1996)) with few modifications. Briefly, a solution of 10 μg of recombinant bFGF plus 5 mg of sucralfate were mixed with 101L1 of Hydron (200 mg/ml in ethanol; New Brunswick, N.J.) and the recombinant protein of interest (2 μg). The suspension was then smeared onto a sterile nylon mesh square (pore size 500 μm; Tetko Inc., Briarcliff Manor, N.Y.) and allowed to dry for 30 min. The fibers of the mesh were pulled to produce pellets of 500 μm³ that were stored at −20° C. Uniformly sized pellets were selected under a microscope and used for the assays.

Mice were anesthetized with Avertin. An incision was made in the cornea using a Nikon SMZ-U dissecting microscope with the aid of a surgical blade. A single pellet was implanted into the pocket. Five days after pellet implantation, corneal angiogenesis was evaluated and photographed.

CAM Assay

Chorioallantoic membrane assays were performed on Leghorn chicken embryos (SPAFAS, MA) at 12-14 days of embryonic development. Matrigel (750 μg/ml), VEGF (250 ng/mesh) and the protein or peptide to be tested were mixed, placed onto nylon meshes (pore size 250 μm; Tetko Inc.) and incubated sequentially at 37° C. for 30 min and at 4° C. for 2 h to induce polymerization. A positive (matrigel and VEGF) and a negative (VEGF alone) control were also prepared for each CAM. Polymerized meshes were placed onto the third outer region of the CAM and incubated for 24 h. To visualize vessels, 4001 μl of fluorescein isothiocyanate dextran (10 mg/ml, SIGMA) was injected in the chick blood stream. After 5-10 min incubation, the chick was topically fixed with 3.7% formaldehyde for 5 min. The meshes were then dissected and mounted onto slides. Fluorescence intensity was analyzed with a computer-assisted image program (NIH Image 1.59).

Peptides used on these assays were synthesized by Chiron (Raleigh, N.C.). Sequence corresponded to amino acids: P—TSP1, 430-447; P-METH1, 549-563; P-METH2, 529-548.

The evaluation of angiogenic or anti-angiogenic responses relies heavily on the sensitivity and specificity of the assays used to assess the response. To evaluate the anti-angiogenic activity of these fragments in vivo, two popular and well-accepted angiogenesis assays were used: the corneal pocket and the chorioallantoic membrane. The visibility, accessibility, and a vascularity of the cornea are highly advantageous and facilitate the visualization of the neovascular response and the topical application of the test substances. A known amount of angiogenesis factor(s) is implanted, as a pellet, in a pocket made in the cornea eye. To test an angiogenesis inhibitor, the molecule is implanted with the stimulator in the same pellet, and the response is compared to the stimulator alone.

In these experiments, bFGF was used as the vascularization stimulator. Pellets containing the recombinant protein were implanted in mouse corneas and their ability to inhibit the bFGF-induced angiogenic response was compared to that of controls. When a bFGF pellet containing GST was implanted new capillary vessels grew from the cornea limbus, across the cornea and into the pellet within 5 days. In contrast, addition of GST-METH1 or GST-METH2 to the bFGF pellets completely abolished blood vessel growth. Table 4 contains a summary of the results obtained from 41 assays performed. Intact TSP1 purified from platelets and GST-TSP1 were used as positive controls. All assays were performed at identical concentrations, suggesting that METH1 and METH2 have similar potency to that of TSP1 in the inhibition of angiogenesis. In addition, when half of the standard concentration was used, a weak, however noticeable response was seen, indicating a dose-dependent effect.

TABLE 4
Activity of METH1 and METH2 recombinant proteins in the
corneal pocket assay

| bFGF Pellets | Vascularized corneas/Total corneas |
|---|---|
| Vehicle | 5/5 |
| TSP1 | 0/5 |
| GST | 11/11 |
| GST-TSP1-TI | 1/4 |
| GST-METH1-TSP | 0/8 |
| GST-METH2-TSP | 0/8 |

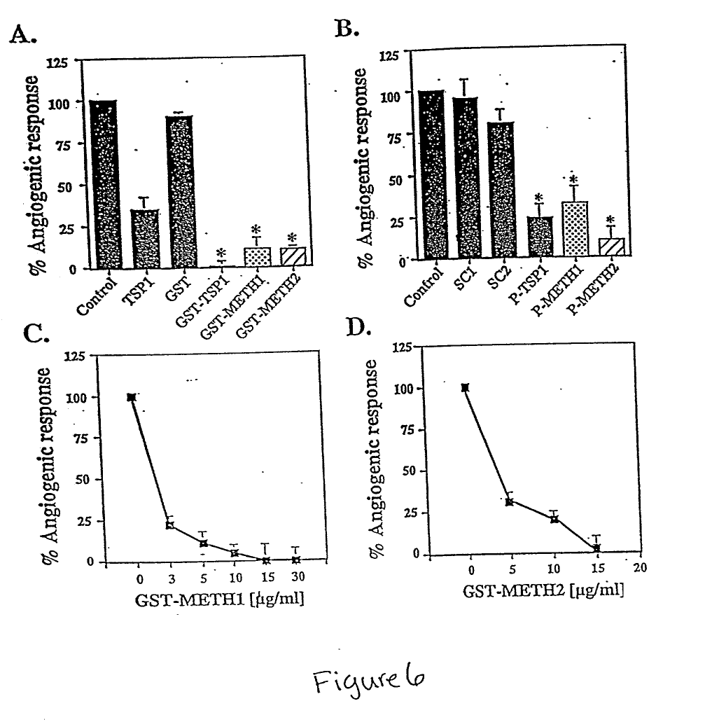

In the CAM assay, the angiogenic response is analyzed by measuring the number of vessels that grow within a matrix polymer containing the angiogenic growth factor. To determine whether recombinant METH1 and METH2 proteins inhibited neovascularization in the CAM assay induced by VEGF, a matrigel polymer containing VEGF and the recombinant protein were implanted in the CAM. Quantitative analysis of the experiments, which included three different polymers per treatment are shown in FIG. 6A. Matrigels polymers containing VEGF plus 5 μg of GST-METH1 or GST-METH2 caused greater than 80% inhibition in blood vessel growth. A similar potency was found using the GST recombinant protein derived from the type I repeats of TSP1. Furthermore, the anti-angiogenic effect of the TSP domains in METH1 and METH2 was dose-dependent with a complete inhibition of blood vessel growth when 15 μg/ml of protein was used (FIGS. 6C and D). GST alone, at identical concentrations, had no significant effect on VEGF-stimulated angiogenesis. CAM assays performed with the unprocessed form of METH-1 and METH2 provided similar results to the processed forms. It was unclear whether processing is not required for function or if the CAM tissue lead to processing of our proteins. Thus, the intact protein was incubated with CAM tissue for 24 h and was evaluated the protein on Western blots. The results demonstrate that the CAM tissue was able to generate a 68 kD METH1 processed protein.

Synthetic peptides from the second or the third type I repeats of human TSP1 can mimic the anti-angiogenic effects of the intact TSP1 (Tolsma, S. S., et al., J. Cell. Biol. 122:497-511 (1993)). In fact, a 19-residue polypeptide was shown to be sufficient to block in vivo neovascularization in the rat cornea and to inhibit the bFGF-induced migration of cultured endothelial cells (Vogel, T., et al., J. Cell. Biochem. 53:74-84 (1993); Tolsma, S. S., et al., J. Cell. Biol. 122:497-511 (1993)). To test whether the same was true for the METH1 and METH2 TSP domains, peptides derived from the same region were synthesized and their anti-angiogenic activity was evaluated in the CAM assay. The results are shown in FIG. 6B. Peptides derived from both the TSP domain of METH1 and METH2 blocked VEGF-induced angiogenesis similarly to that of TSP1. In contrast, scramble peptides had no significant effects.

### Example 5

### Proliferation Assays

Human dermal endothelial cells (HDEC) were isolated and grown on Vitrogen™ coated petri-dishes in EBM (Clonetics, San Diego, Calif.) supplemented with 15% fetal calf serum, 25 μg/ml cAMP, and 1 μg/ml of hydrocortisone-21-acetate and were used from passages 3 to 6. Cells were made quiescent by incubation of confluent monolayers with phenol red-free EBM containing 0.2% BSA for 48 h. Human dermal fibroblasts were isolated from neonatal foreskin and by enzymatic dissociation. Both fibroblasts and smooth muscle cells were maintained in DMEM supplemented with 10% fetal calf serum. Human mammary epithelial cells (HMEC) were purchased from Clonetics and maintained in the recommended media (mammary epithelial growth media, MEGM).

Quiescent human dermal endothelial cells, between passage 3 and 6, were plated on vitrogen™ coated 24-well plates in EBM supplemented with 0.2% BSA, 0.1% fetal calf serum and 1 ng/ml of bFGF in the presence or absence of the recombinant protein and incubated at 5% CO₂ at 37° C. for 48 h. For vascular smooth muscle (VSM) and fibroblast proliferation assays, cells were incubated under the same conditions but using DMEM instead of EBM. Human mammary epithelial cells were incubated on their growth media. A pulse of [³H]-Thymidine (1 μCi/μl) was added during the last 4 h prior harvesting. Cells were washed and fixed in 10% TCA. Incorporation of [³H]-thymidine was determined by scintillation counting, as previously described (Iruela-Arispe, M. L. & Sage, E. H., J. Cell. Biochem. 52:414 (1993)).

Statistical analyses were done using In-Stat software (Graph Pad Software) for Macintosh. Assuming normal distributions, data were analyzed by one-way ANOVA, followed by either T-test Dunnett test for comparisons between groups, or student-Newman-Kleus test for multiple comparisons between groups.

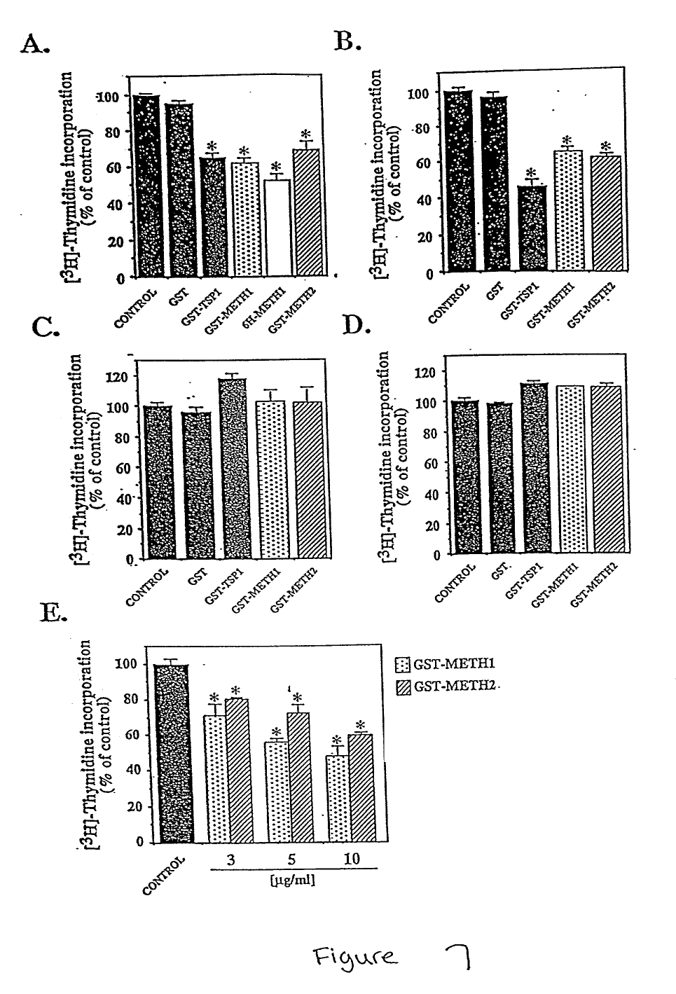

To gain insight into the mechanism by which METH1 and METH2 inhibit neovascularization, the direct effect of the purified recombinant fusion proteins on endothelial cell proliferation was tested. Serum-starved endothelial cells were plated into growth medium containing bFGF and FCS. Recombinant proteins (3 μg/ml) were added at the same time of plating. 40% (GST-METH1), 45% (6H-GST) or 36% (GST-METH2) inhibition was observed, in contrast to a non-significant effect when GST alone was added. The recombinant protein from the type I repeats of TSP1 had similar inhibitory effects. (FIG. 7A). Furthermore, suppression of proliferation mediated by METH1 or METH2 was dose-dependent, as shown in FIG. 7E. The inhibition was observed as early as one day after treatment and the inhibitory effect was not toxic and reversible since the removal of -the recombinant protein and subsequent addition of growth factor alone led to the resumption of endothelial cell proliferation.

The cell specificity of the anti-proliferative effects for METH1 and METH2 on the endothelium was evaluated by additional proliferation assays on a variety of non-endothelial cells. No significant inhibition of proliferation was seen on fibroblasts or smooth muscle cell cultures. In contrast, a non significant, but reproducible stimulation of proliferation for these two cell types could be observed. This result rules out the presence of any potential nonspecific inhibitor of cell growth in the recombinant protein preparations. On mammary epithelial cell, however, METH1 and METH2 inhibited cell proliferation to the same degree as endothelial cells. Interestingly, TSP1 also suppresses mammary epithelial cell proliferation both in vitro and in a transgenic model.

The possibility that METH1 and METH2 might act as disintegrins is consistent with their anti-angiogenic properties. Clearly blockade of αvβ3 and β1 integrins with antibodies has been shown to inhibit neovascularization both during development and in tumors (Brooks, P. C., et al., Cell 85:683-693 (1996); Brooks, P. C., et al., Cell 92:391-400 (1998); Senger, D. R., et al., Proc. Natl. Acad. Sci. USA 94:13612-13617 (1997)). Integrins are essential for the mediation of both proliferative and migratory signals (Schwartz, M. A. & Ingber, D. E., Mol. Biol. Cell 5:389-393 (1994)), therefore interference with those signals can be highly deleterious to the angiogenic process. The angiogenic functional assays were performed with recombinant protein containing only the type I repeats in METH1 and METH2.

The mechanism of action of METH1 and METH2 with regards to their angio-inhibitory activity is not known. To date we have evidence that these proteins are secreted and bind to endothelial cells. Further investigations are guided towards the identification of receptors and signal transduction mechanisms. A likely hypothesis resulting from the lessons learned from TSP1 is that both METH1 and METH2 bind to CD36. Recently, this scavenger receptor has been implicated in the mediation of signals by which TSP-1 exert its anti-angiogenic effects (Dawson, D. W., et al., J. Cell. Biol. 138:707-717 (1997)). Both the CSVTCG (SEQ ID NO:83) (Asch, A. S., et al., Nature 262:1436-1439 (1993); Catimel, B., et al., Biochem. J. 284:231-236 (1992)) and the GCQXR (SEQ ID NO:84) sequences have been proposed as primary binding motifs to CD36 (Dawson, D. W., et al., J. Cell. Biol. 138:707-717 (1997)). METH1 and METH2 have almost entire conservation in both these regions. A complementary and also likely occurrence is binding of METH1 and METH2 to bFGF. Binding to heparin and bFGF has been proposed as part of the anti-angiogenic activity of TSP1 (Guo, N., et al., J. Peptide Res. 49 (1997)). This property appears to be mediated through the WSXWS (SEQ ID NO: 82) motif, also conserved in METH1 and METH2. Future efforts will focus on the signals implicated in the anti-angiogenic properties mediated by these novel proteins and on their potential as proteases of the extracellular milieu.

### Example 6

### Isolation of the METH1 or METH2 cDNA Clone From the Deposited Sample

Two approaches can be used to isolate METH1 or METH2 from the deposited sample. First, the deposited clone is transformed into a suitable host (such as XL-1 Blue (Stratagene)) using techniques known to those of skill in the art, such as those provided by the vector supplier or in related publications or patents. The transformants are plated on 1.5% agar plates (containing the appropriate selection agent, e.g., ampicillin) to a density of about 150 transformants (colonies) per plate. A single colony is then used to generate DNA using nucleic acid isolation techniques well known to those skilled in the art. (e.g., Sambrook et al., Molecular Cloning: A Laboratory Manual, 2nd Edit., (1989), Cold Spring Harbor Laboratory Press.)

Alternatively, two primers of 17-20 nucleotides derived from both ends of the SEQ ID NO: 1 or SEQ ID NO:3 (i.e., within the region of SEQ ID NO: 1 or SEQ ID NO:3 bounded by the 5′ NT and the 3′ NT of the clone) are synthesized and used to amplify the METH1 or METH2 cDNA using the deposited cDNA plasmids as templates. The polymerase chain reaction is carried out under routine conditions, for instance, in 25 μl of reaction mixture with 0.5 μg of the above cDNA template. A convenient reaction mixture is 1.5-5 MM MgCl₂, 0.01% (w/v) gelatin, 20 μM each of dATP, dCTP, dGTP, dTTP, 25 pmol of each primer and 0.25 Unit of Taq polymerase. Thirty five cycles of PCR (denaturation at 94° C. for 1 min; annealing at 55° C. for 1 min; elongation at 72° C. for 1 min) are performed with a Perkin-Elmer Cetus automated thermal cycler. The amplified product is analyzed by agarose gel electrophoresis and the DNA band with expected molecular weight is excised and purified. The PCR product is verified to be the selected sequence by subcloning and sequencing the DNA product.

Several methods are available for the identification of the 5′ or 3′ non-coding portions of the METH1 or METH2 gene which may not be present in the deposited clones. These methods include but are not limited to, filter probing, clone enrichment using specific probes, and protocols similar or identical to 5′ and 3 “RACE” protocols which are well known in the art. For instance, a method similar to 5′ RACE is available for generating the missing 5′ end of a desired full-length transcript. (Fromont-Racine et al., Nucleic Acids Res. 21(7):1683-1684 (1993).) Briefly, a specific RNA oligonucleotide is ligated to the 5′ ends of a population of RNA presumably containing full-length gene RNA transcripts. A primer set containing a primer specific to the ligated RNA oligonucleotide and a primer specific to a known sequence of the METH1 or METH2 gene of interest is used to PCR amplify the portion of the METH1 or METH2 full-length gene. This amplified product may then be sequenced and used to generate the full length gene.

This above method starts with total RNA isolated from the desired source, although poly-A+ RNA can be used. The RNA preparation can then be treated with phosphatase if necessary to eliminate 5′ phosphate groups on degraded or damaged RNA which may interfere with the later RNA ligase step. The phosphatase should then be inactivated and the RNA treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase.

This modified RNA preparation is used as a template for first strand cDNA synthesis using a gene specific oligonucleotide. The first strand synthesis reaction is used as a template for PCR amplification of the desired 5′ end using a primer specific to the ligated RNA oligonucleotide and a primer specific to the known sequence of the gene of interest. The resultant product is then sequenced and analyzed to confirm that the 5′ end sequence belongs to the METH1 or METH2 gene.

### Example 7

### Bacterial Expression of METH1 or METH2

A METH1 or METH2polynucleotide encoding a METH1 or METH2polypeptide invention is amplified using PCR oligonucleotide primers corresponding to the 5′ and 3′ ends of the DNA sequence, as outlined in Example 5, to synthesize insertion fragments. The primers used to amplify the cDNA insert should preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif.). This plasmid vector encodes antibiotic resistance (Amp^{r}), a bacterial origin of replication (ori), an IPTG-regulatable promoter/operator (P/O), a ribosome binding site (RBS), a 6-histidine tag (6-His), and restriction enzyme cloning sites. The pQE-9 vector is digested with BamHI and XbaI and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, which expresses the lacI repressor and also confers kanamycin resistance (Kan^{r}). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 ug/ml) and Kan (25 ug/ml). The ON culture is used to inoculate a large culture at a ratio of 1:100 to 1:250. The cells are grown to an optical density 600 (O.D.⁶⁰⁰) of between 0.4 and 0.6. IPTG (Isopropyl-B-D-thiogalacto pyranoside) is then added to a final concentration of 1 mM. IPTG induces by inactivating the lacI repressor, clearing the P/O leading to increased gene expression.

Cells are grown for an extra 3 to 4 hours. Cells are then harvested by centrifugation (20 mins at 6000× g). The cell pellet is solubilized in the chaotropic agent 6 Molar Guanidine HCl by stirring for 34 hours at 4° C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Proteins with a 6× His tag bind to the Ni-NTA resin with high affinity and can be purified in a simple one-step procedure (for details see: The QIA expressionist (1995) QIAGEN, Inc., supra). Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified METH1 or METH2 protein is then renatured by dialyzing it against phosphate-buffered saline (PBS) or 50 mM Na-acetate, pH 6 buffer plus 200 mM NaCl. Alternatively, the METH1 or METH2 protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins are eluted by the addition of 250 mM immidazole. Immidazole is removed by a final dialyzing step against PBS or 50 mM sodium acetate pH 6 buffer plus 200 mM NaCl. The purified METH1 or METH2 protein is stored at 4° C. or frozen at −80° C.

In addition to the above expression vector, the present invention further includes an expression vector comprising phage operator and promoter elements operatively linked to a METH1 or METH2 polynucleotide, called pHE4a. (ATCC Accession Number 209645, deposited Feb. 25, 1998.) This vector contains: 1) a neomycinphosphotransferase gene as a selection marker, 2) an E. coli origin of replication, 3) a T5 phage promoter sequence, 4) two lac operator sequences, 5) a Shine-Delgarno sequence, and 6) the lactose operon repressor gene (lacIq). The origin of replication (oriC) is derived from pUC19 (LTI, Gaithersburg, Md.). The promoter sequence and operator sequences are made synthetically. DNA can be inserted into the pHEa by restricting the vector with NdeI and XbaI, BamII, XhoI, or Asp718, running the restricted product on a gel, and isolating the larger fragment (the stuffer fragment should be about 310 base pairs). The DNA insert is generated according to the PCR protocol described in Example 5, using PCR primers having restriction sites for NdeI (5′ primer) and XbaI, BamHI, XhoI, or Asp718 (3′ primer). The PCR insert is gel purified and restricted with compatible enzymes. The insert and vector are ligated according to standard protocols.

The engineered vector could easily be substituted in the above protocol to express protein in a bacterial system.

### Example 8

### Purification of METH1 or METH2 Polypeptide from an Inclusion Body

The following alternative method can be used to purify METH1 or METH2 polypeptide expressed in E. coli when it is present in the form of inclusion bodies. Unless otherwise specified, all of the following steps are conducted at 4-10° C.

Upon completion of the production phase of the E. coli fermentation, the cell culture is cooled to 4-10° C. and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfuidics, Corp. or APV Gaulin, Inc.) twice at 4000-6000 psi. The homogenate is then mixed with NaCl solution to a final concentration of 0.5 M NaCl, followed by centrifugation at 7000× g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 24 hours. After 7000× g centrifugation for 15 min., the pellet is discarded and the polypeptide containing supernatant is incubated at 4° C. overnight to allow further GuHCl extraction.

Following high speed centrifugation (30,000× g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4° C. without mixing for 12 hours prior to further purification steps.

To clarify the refolded polypeptide solution, a previously prepared tangential filtration unit equipped with 0.16 urn membrane filter with appropriate surface area (e.g., Filtron), equilibrated with 40 mM sodium acetate, pH 6.0 is employed. The filtered sample is loaded onto a cation exchange resin (e.g., Poros HS-50, Perseptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the METH1 or METH2 polypeptide are then pooled and mixed with 4 volumes of water. The diluted sample is then loaded onto a previously prepared set of tandem columns of strong anion (Poros HQ-50, Perseptive Biosystems) and weak anion (Poros CM-20, Perseptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A₂₈₀ monitoring of the effluent. Fractions containing the polypeptide (determined, for instance, by 16% SDS-PAGE) are then pooled.

The resultant METH1 or METH2 polypeptide should exhibit greater than 95% purity after the above refolding and purification steps. No major contaminant bands should be observed from Coomassie blue stained 16% SDS-PAGE gel when 5 μg of purified protein is loaded. The purified METH1 or METH2 protein can also be tested for endotoxin/LPS contamination, and typically the LPS content is less than 0.1 ng/ml according to LAL assays.

### Example 9

### Cloning and Expression of METH1 or METH2 in a Baculovirus Expression System

In this example, the plasmid shuttle vector pA2 is used to insert METH1 or METH2 polynucleotide into a baculovirus to express METH1 or METH2. This expression vector contains the strong polyhedrin promoter of the Autographa californica nuclear polyhedrosis virus (AcMNPV) followed by convenient restriction sites such as BamHI, XbaI and Asp718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that expresses the cloned METH1 or METH2 polynucleotide.

Many other baculovirus vectors can be used in place of the vector above, such as pAc373, pVL941, and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

Specifically, the METH1 or METH2 cDNA sequence contained in the deposited clone, including the AUG initiation codon and any naturally associated leader sequence, is amplified using the PCR protocol described in Example 5. If the naturally occurring signal sequence is used to produce the secreted protein, the pA2 vector does not need a second signal peptide. Alternatively, the vector can be modified (pA2 GP) to include a baculovirus leader sequence, using the standard methods described in Summers et al., “A Manual of Methods for Baculovirus Vectors and Insect Cell Culture Procedures,” Texas Agricultural Experimental Station Bulletin No. 1555 (1987).

The amplified fragment is isolated from a 1% agarose gel using a commercially available kit (“Geneclean,” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB 101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five ug of a plasmid containing the polynucleotide is co-transfected with 1.0 ug of a commercially available linearized baculovirus DNA (“BaculoGolda baculovirus DNA”, Pharmingen, San Diego, Calif.), using the lipofection method described by Felgner et al., Proc. Natl. Acad. Sci. USA 84:7413-7417 (1987). One ug of BaculoGold^{a} virus DNA and 5 ug of the plasmid are mixed in a sterile well of a microtiter plate containing 50 μl of serum-free Grace's medium (Life Technologies Inc., Gaithersburg, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27° C. The transfection solution is then removed from the plate and 1 ml of Grace's insect medium supplemented with 10% fetal calf serum is added. Cultivation is then continued at 27° C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4° C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 uCi of ³⁵S-methionine and 5 uCi ³⁵S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the produced METH1 or METH2 protein.

### Example 10

### Expression of METH1 or METH2 in Mammalian Cells

METH1 or METH2 polypeptide can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2DHFR (ATCC 37146), pBC 12MI (ATCC 67109), pCMVSport 2.0, and pCMVSport 3.0. Mammalian host cells that could be used include, human Hela, 293, H9 and Jurkat cells, mouse NH3T3 and C127 cells, Cos 1, Cos 7 and CV1, quail QC1-3 cells, mouse L cells and Chinese hamster ovary (CHO) cells.

Alternatively, METH1 or METH2 polypeptide can be expressed in stable cell lines containing the METH1 or METH2 polynucleotide integrated into a chromosome. The co-transfection with a selectable marker such as DHFR, gpt, neomycin, hygromycin allows the identification and isolation of the transfected cells.

The transfected METH1 or METH2 gene can also be amplified to express large amounts of the encoded protein. The DHFR (dihydrofolate reductase) marker is useful in developing cell lines that carry several hundred or even several thousand copies of the gene of interest. (See, e.g., Alt, F. W., et al., J. Biol. Chem. 253:1357-1370 (1978); Hamlin, J. L. and Ma, C., Biochem. et Biophys. Acta 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

Derivatives of the plasmid pSV2-DHFR (ATCC Accession No. 37146), the expression vectors pC4 (ATCC Accession No. 209646) and pC6 (ATCC Accession No.209647) contain the strong promoter (LTR) of the Rous Sarcoma Virus (Cullen et al., Molecular and Cellular Biology, 438-447 (March, 1985)) plus a fragment of the CMV-enhancer (Boshart et al., Cell 41:521-530 (1985).) Multiple cloning sites, e.g., with the restriction enzyme cleavage sites BamHI, XbaI and Asp718, facilitate the cloning of METH1 or METH2. The vectors also contain the 3′ intron, the polyadenylation and termination signal of the rat preproinsulin gene, and the mouse DHFR gene under control of the SV40 early promoter.

If a naturally occurring signal sequence is used to produce a secreted protein, the vector does not need a second signal peptide. Alternatively, if a naturally occurring signal sequence is not used, the vector can be modified to include a heterologous signal sequence in an effort to secrete the protein from the cell. (See, e.g., WO 96/34891.) The amplified fragment is then digested with the appropriate restriction enzyme and purified on a 1% agarose gel using a commercially available kit (“Geneclean,” BIO 101 Inc., La Jolla, Calif.). The isolated fragment and the dephosphorylated vector are then ligated with T4 DNA ligase. E. coli lB 101 or XL-1 Bluecells are then transformed and bacteria are identified that contain the fragment inserted into plasmid pC6 or pC4 using, for instance, restriction enzyme analysis.

Chinese hamster ovary cells lacking an active DHFR gene are used for transfection. Five μg of the expression plasmid pC6 or pC4 is cotransfected with 0.5 μg of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 uM, 2 uM, 5 uM, 10 mM, 20 mM). The same procedure is repeated until clones are obtained which grow at a concentration of 100-200 uM. Expression of METH1 or METH2 is analyzed, for instance, by SDS-PAGE and Western blot or by reversed phase HPLC analysis.

### Example 11

### Construction of N-Terminal and/or C-Terminal Deletion Mutants

The following general approach may be used to clone a N-terminal or C-terminal deletion METH1 or METH2 deletion mutant. Generally, two oligonucleotide primers of about 15-25 nucleotides are derived from the desired 5′ and 3′ positions of a polynucleotide of SEQ ID NO: 1 or SEQ ID NO:3. The 5′ and 3′ positions of the primers are determined based on the desired METH1 or METH2 polynucleotide fragment. An initiation and stop codon are added to the 5′ and 3′ primers respectively, if necessary, to express the METH1 or METH2 polypeptide fragment encoded by the polynucleotide fragment. Preferred METH1 or METH2 polynucleotide fragments are those encoding the N-terminal and C-terminal deletion mutants disclosed above in the “Polynucleotide and Polypeptide Fragments” section of the Specification.

Additional nucleotides containing restriction sites to facilitate cloning of the METH1 or METH2 polynucleotide fragment in a desired vector may also be added to the 5′ and 3′ primer sequences. The METH1 or METH2 polynucleotide fragment is amplified from genomic DNA or from the deposited cDNA clone using the appropriate PCR oligonucleotide primers and conditions discussed herein or known in the art. The METH1 or METH2 polypeptide fragments encoded by the METH1 or METH2 polynucleotide fragments of the present invention may be expressed and purified in the same general manner as the full length polypeptides, although routine modifications may be necessary due to the differences in chemical and physical properties between a particular fragment and full length polypeptide.

As a means of exemplifying but not limiting the present invention, the polynucleotide encoding the METH1 polypeptide fragment R-235 to L-934 or the METH2 polypeptide fragment R-214 to Q-836 is amplified and cloned as follows: A 5′ primer is generated comprising a restriction enzyme site followed by an initiation codon in frame with the polynucleotide sequence encoding the N-terminal portion of the polypeptide fragment beginning with R-235 or R-214, respectively. A complementary 3′ primer is generated comprising a restriction enzyme site followed by a stop codon in frame with the polynucleotide sequence encoding C-terminal portion of the METH1 or METH2 polypeptide fragment ending with L-934 or Q-836, respectively.

The amplified polynucleotide fragment and the expression vector are digested with restriction enzymes which recognize the sites in the primers. The digested polynucleotides are then ligated together. The METH1 or METH2 polynucleotide fragment is inserted into the restricted expression vector, preferably in a manner which places the METH1 or METH2 polypeptide fragment coding region downstream from the promoter. The ligation mixture is transformed into competent E. coli cells using standard procedures and as described in the Examples herein. Plasmid DNA is isolated from resistant colonies and the identity of the cloned DNA confirmed by restriction analysis, PCR and DNA sequencing.

### Example 12

### Protein Fusions of METH1 or METH2

METH1 or METH2 polypeptides are preferably fused to other proteins. These fusion proteins can be used for a variety of applications. For example, fusion of METH1 or METH2 polypeptides to His-tag, HA-tag, protein A, IgG domains, and maltose binding protein facilitates purification. (See Example 7; see also EP A 394,827; Traunecker, et al., Nature 331:84-86 (1988).) Similarly, fusion to IgG-1, IgG-3, and albumin increases the half-life time in vivo. Nuclear localization signals fused to METH1 or METH2 polypeptides can target the protein to a specific subcellular localization, while covalent heterodimer or homodimers can increase or decrease the activity of a fusion protein. Fusion proteins can also create chimeric molecules having more than one function. Finally, fusion proteins can increase solubility and/or stability of the fused protein compared to the non-fused protein. All of the types of fusion proteins described above can be made by modifying the following protocol, which outlines the fusion of a polypeptide to an IgG molecule, or the protocol described in Example 7.

Briefly, the human Fc portion of the IgG molecule can be PCR amplified, using primers that span the 5′ and 3′ ends of the sequence described below. These primers also should have convenient restriction enzyme sites that will facilitate cloning into an expression vector, preferably a mammalian expression vector.

For example, if pC4 (Accession No. 209646) is used, the human Fc portion can be ligated into the BamHI cloning site. Note that the 3′ BamHI site should be destroyed. Next, the vector containing the human Fc portion is re-restricted with BamHI, linearizing the vector, and METH1 or METH2 polynucleotide, isolated by the PCR protocol described in Example 5, is ligated into this BamHI site. Note that the polynucleotide is cloned without a stop codon, otherwise a fusion protein will not be produced.

If the naturally occurring signal sequence is used to produce the secreted protein, pC4 does not need a second signal peptide. Alternatively, if the naturally occurring signal sequence is not used, the vector can be modified to include a heterologous signal sequence. (See, e.g., WO 96/34891.)

Human IgG Fc region:

Human IgG Fc region:

(SEQ ID NO:85)

GGGATCCGGAGCCCAAATCTTCTGACAAAACTCACACATGCCCACCGTGC
CCAGCACCTGAATTCGAGGGTGCACCGTCAGTCTTCCTCTTCCCCCCAAA
ACCCAAGGACACCCTCATGATCTCCCGGACTCCTGAGGTCACATGCGTGG
TGGTGGACGTAAGCCACGAAGACCCTGAGGTCAAGTTCAACTGGTACGTG
GACGGCGTGGAGGTGCATAATGCCAAGACAAAGCCGCGGGAGGAGCAGTA
CAACAGCACGTACCGTGTGGTCAGCGTCCTCACCGTCCTGCACCAGGACT
GGCTGAATGGCAAGGAGTACAAGTGCAAGGTCTCCAACAAAGCCCTCCCA
ACCCCCATCGAGAAAACCATCTCCAAAGCCAAAGGGCAGCCCCGAGAAC
CACAGGTGTACACCCTGCCCCCATCCCGGGATGAGCTGACCAAGAACCAG
GTCAGCCTGACCTGCCTGGTCAAAGGCTTCTATCCAAGCGACATCGCCGT
GGAGTGGGAGAGCAATGGGCAGCCGGAGAACAACTACAAGACCACGCCT
CCCGTGCTGGACTCCGACGGCTCCTTCTTCCTCTACAGCAAGCTCACCGT
GGACAAGAGCAGGTGGCAGCAGGGGAACGTCTTCTCATGCTCCGTGATGC
ATGAGGCTCTGCACAACCACTACACGCAGAAGAGCCTCTCCCTGTCTCCG
GGTAAATGAGTGCGACGGCCGCGACTCTAGAGGAT

### Example 13

### Production of an Antibody

a) The antibodies of the present invention can be prepared by a variety of methods. (See, Current Protocols, Chapter 2.) For example, cells expressing METH1 or METH2 is administered to an animal to induce the production of sera containing polyclonal antibodies. In a preferred method, a preparation of METH1 or METH2 protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp.563-681 (1981).) In general, such procedures involve immunizing an animal (preferably a mouse) with METH1 or METH2 polypeptide or, more preferably, with a secreted METH1 or METH2 polypeptide-expressing cell. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100 ug/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP20), available from the ATCC. After fusion, the resulting hybridoma cells are selectively maintained in HAT medium, and then cloned by limiting dilution as described by Wands et al. (Gastroenterology 80:225-232 (1981).) The hybridoma cells obtained through such a selection are then assayed to identify clones which secrete antibodies capable of binding the METH1 or METH2 polypeptide.

Alternatively, additional antibodies capable of binding to METH1 or METH2 polypeptide can be produced in a two-step procedure using anti-idiotypic antibodies. Such a method makes use of the fact that antibodies are themselves antigens, and therefore, it is possible to obtain an antibody which binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the METH1 or METH2 protein-specific antibody can be blocked by METH1 or METH2. Such antibodies comprise anti-idiotypic antibodies to the METH1 or METH2 protein-specific antibody and can be used to immunize an animal to induce formation of further METH1 or METH2 protein-specific antibodies.

It will be appreciated that Fab and F(ab′)₂ and other fragments of the antibodies of the present invention may be used according to the methods disclosed herein. Such fragments are typically produced by proteolytic cleavage, using enzymes such as papain (to produce Fab fragments) or pepsin (to produce F(ab′)₂ fragments). Alternatively, secreted METH1 or METH2 protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

b) Isolation of antibody fragments directed against METH1 and/or METH2 from a library of scFvs.

Naturally occurring V-genes isolated from human PBLs are constructed into a large library of antibody fragments which contain reactivities against METH1 and/or METH2 to which the donor may or may not have been exposed (see e.g., U.S. Pat. No. 5,885,793 incorporated herein in its entirety by reference).

Rescue of the Library. A library of scFvs is constructed from the RNA of human PBLs as described in WO92101047. To rescue phage displaying antibody fragments, approximately 10⁹ E. coli harbouring the phagemid are used to inoculate 50 ml of 2× TY containing 1% glucose and 100 μg/ml of ampicillin (2× TY-AMP-GLU) and grown to an O.D. of 0.8 with shaking. Five ml of this culture is used to innoculate 50 ml of 2× TY-AMP-GLU, 2×10⁸ TU of delta gene 3 helper (M13 delta gene III, see WO92/01047) are added and the culture incubated at 37° C. for 45 minutes without shaking and then at 37° C. for 45 minutes with shaking. The culture is centrifuged at 4000 r.p.m. for 10 min. and the pellet resuspended in 2 liters of 2× TY containing 100 μg/ml ampicillin and 50 μg/ml kanamycin and grown overnight. Phage are prepared as described in WO92/01047.

M13 delta gene III is prepared as follows: M13 delta gene III helper phage does not encode gene III protein, hence the phage(mid) displaying antibody fragments have a greater avidity of binding to antigen. Infectious M13 delta gene III particles are made by growing the helper phage in cells harbouring a pUC 19 derivative supplying the wild type gene III protein during phage morphogenesis. The culture is incubated for 1 hour at 37° C. without shaking and then for a further hour at 37° C. with shaking. Cells are spun down (IEC-Centra 8,4000 revs/min for 10 min), resuspended in 300 ml 2× TY broth containing 100 μg ampicillin/ml and 25 μg kanamycin/ml (2× TY-AMP-KAN) and grown overnight, shaking at 37° C. Phage particles are purified and concentrated from the culture medium by two PEG-precipitations (Sambrook et al., 1990), resuspended in 2 ml PBS and passed through a 0.45 μm filter (Minisart NML; Sartorius) to give a final concentration of approximately 1013 transducing units/ml (ampicillin-resistant clones).

Panning of the Library. Immunotubes (Nunc) are coated overnight in PBS with 4 ml of either 100 μg/ml or 10 μg/ml of a polypeptide of the present invention. Tubes are blocked with 2% Marvel-PBS for 2 hours at 37° C. and then washed 3 times in PBS. Approximately 10¹³ of phage is applied to the tube and incubated for 30 minutes at room temperature tumbling on an over and under turntable and then left to stand for another 1.5 hours. Tubes are washed 10 times with PBS 0.1% Tween-20 and 10 times with PBS. Phage are eluted by adding 1 ml of 100 mM triethylamine and rotating 15 minutes on an under and over turntable after which the solution is immediately neutralized with 0.5 ml of 1.0M Tris-HCl, pH 7.4. Phage are then used to infect 10 ml of mid-log E. coli TG1 by incubating eluted phage with bacteria for 30 minutes at 37° C. The E. coli are then plated on TYE plates containing 1% glucose and 100 μg/ml ampicillin. The resulting bacterial library is then rescued with delta gene 3 helper phage as described above to prepare phage for a subsequent round of selection. This process is then repeated for a total of 4 rounds of affinity purification with tube-washing increased to 20 times with PBS, 0.1% Tween-20 and 20 times with PBS for rounds 3 and 4.

Characterization of Binders. Eluted phage from the 3rd and 4th rounds of selection are used to infect E. coli HB 2151 and soluble scFv is produced (Marks, et al., 1991) from single colonies for assay. ELISAs are performed with microtitre plates coated with either 10 μg/ml of the polypeptide of the present invention in 50 mM bicarbonate pH 9.6. Clones positive in ELISA are further characterized by PCR fingerprinting (see e.g., WO92/01047) and then by sequencing.

### Example 14

### Production Of METH1 or METH2 Protein For High-Throughput Screening Assays

The following protocol produces a supernatant containing METH1 or METH2 polypeptide to be tested. This supernatant can then be used in the Screening Assays described in Examples 16-23.

First, dilute Poly-D-Lysine (644 587 Boehringer-Mannheim) stock solution (1 mg/ml in PBS) 1:20 in PBS (w/o calcium or magnesium 17-516F Biowhittaker) for a working solution of 50 μg/ml. Add 200 μl of this solution to each well (24 well plates) and incubate at RT for 20 minutes. Be sure to distribute the solution over each well (note: a 12-channel pipetter may be used with tips on every other channel). Aspirate off the Poly-D-Lysine solution and rinse with 1 ml PBS (Phosphate Buffered Saline). The PBS should remain in the well until just prior to plating the cells and plates may be poly-lysine coated in advance for up to two weeks.

Plate 293T cells (do not carry cells past P+20) at 2×10⁵ cells/well in 0.5 ml DMEM(Dulbecco's Modified Eagle Medium)(with 4.5 G/L glucose and L-glutamine (12-604F Biowhittaker))/10% heat inactivated FBS(14-503F Biowhittaker)/1× Penstrep(17-602E Biowhittaker). Let the cells grow overnight.

The next day, mix together in a sterile solution basin: 300 μl Lipofectamine (18324-012 Gibco/BRL) and 5 ml Optimem 1 (31985070 Gibco/BRL)/96-well plate. With a small volume multi-channel pipetter, aliquot approximately 2 μg of an expression vector containing a polynucleotide insert, produced by the methods described in Examples 10-12, into an appropriately labeled 96-well round bottom plate. With a multi-channel pipetter, add 50 μl of the Lipofectamine/Optimem I mixture to each well. Pipette up and down gently to mix. Incubate at RT 15-45 minutes. After about 20 minutes, use a multi-channel pipetter to add 150 μl Optimem I to each well. As a control, one plate of vector DNA lacking an insert should be transfected with each set of transfections.

Preferably, the transfection should be performed by tag-teaming the following tasks. By tag-teaming, hands on time is cut in half, and the cells do not spend too much time on PBS. First, person A aspirates off the media from four 24-well plates of cells, and then person B rinses each well with 0.5-1 ml PBS. Person A then aspirates off PBS rinse, and person B, using a 12-channel pipetter with tips on every other channel, adds the 200 ul of DNA/Lipofectamine/Optimem I complex to the odd wells first, then to the even wells, to each row on the 24-well plates. Incubate at 37° C. for 6 hours.

While cells are incubating, prepare appropriate media, either 1% BSA in DMEM with 1× penstrep, or HGS CHO-5 media (116.6 mg/L of CaCl₂ (anhyd); 0.00130 mg/L CuSO₄-5H₂O; 0.050 mg/L of Fe(NO₃)₃-9H₂O; 0.417 mg/L of FeSO₄.7H₂O; 311.80 mg/L of KCl; 28.64 mg/L of MgCl₂; 48.84 mg/L of MgSO₄; 6995.50 mg/L of NaCl; 2400.0 mg/L of NaHCO₃; 62.50 mg/L of NaH₂PO₄—H₂O; 71.02 mg/L of Na₂HPO₄; 0.4320 mg/L of ZnSO₄.7H₂O; 0.002 mg/L of Arachidonic Acid; 1.022 mg/L of Cholesterol; 0.070 mg/L of DL-alpha-Tocopherol-Acetate; 0.0520 mg/L of Linoleic Acid; 0.010 mg/L of Linolenic Acid; 0.010 mg/L of Myristic Acid; 0.010 mg/L of Oleic Acid; 0.010 mg/L of Palmitric Acid; 0.010 mg/L of Palmitic Acid; 100 mg/L of Pluronic F-68; 0.010 mg/L of Stearic Acid; 2.20 mg/L of Tween 80; 4551 mg/L of D-Glucose; 130.85 mg/ml of L-Alanine; 147.50 mg/ml of L—Arginine-HCL; 7.50 mg/ml of L-Asparagine-H₂O; 6.65 mg/ml of L-Aspartic Acid; 29.56 mg/ml of L-Cystine-2HCL—H₂O; 31.29 mg/ml of L-Cystine-2HCL; 7.35 mg/ml of L-Glutamic Acid; 365.0 mg/ml of L-Glutamine; 18.75 mg/ml of Glycine; 52.48 mg/ml of L-Histidine-HCL—H₂O; 106.97 mg/ml of L-Isoleucine; 111.45 mg/ml of L-Leucine; 163.75 mg/ml of L-Lysine HCL; 32.34 mg/ml of L-Methionine; 68.48 mg/mil of L-Phenylalainine; 40.0 mg/ml of L-Proline; 26.25 mg/ml of L-Serine; 101.05 mg/ml of L-Threonine; 19.22 mg/ml of L-Tryptophan; 91.79 mg/ml of L-Tryrosine-2Na-2H₂O; and 99.65 mg/ml of L-Valine; 0.0035 mg/L of Biotin; 3.24 mg/L of D-Ca Pantothenate; 11.78 mg/L of Choline Chloride; 4.65 mg/L of Folic Acid; 15.60 mg/L of i-Inositol; 3.02 mg/L of Niacinamide; 3.00 mg/L of Pyridoxal HCL; 0.031 mg/L of Pyridoxine HCL; 0.319 mg/L of Riboflavin; 3.17 mg/L of Thiamine HCL; 0.365 mg/L of Thymidine; 0.680 mg/L of Vitamin B₁₂; 25 mM of HEPES Buffer; 2.39 mg/L of Na Hypoxanthine; 0.105 mg/L of Lipoic Acid; 0.081 mg/L of Sodium Putrescine-2HCL; 55.0 mg/L of Sodium Pyruvate; 0.0067 mg/L of Sodium Selenite; 20 uM of Ethanolamine; 0.122 mg/L of Ferric Citrate; 41.70 mg/L of Methyl-B-Cyclodextrin complexed with Linoleic Acid; 33.33 mg/L of Methyl-B-Cyclodextrin complexed with Oleic Acid; 10 mg/L of Methyl-B-Cyclodextrin complexed with Retinal Acetate. Adjust osmolarity to 327 mOsm with 2 mm glutamine and 1× penstrep. (BSA (81-068-3 Bayer) 100gm dissolved in 1L DMEM for a 10% BSA stock solution). Filter the media and collect 50 μl for endotoxin assay in 15 ml polystyrene conical.

The transfection reaction is terminated, preferably by tag-teaming, at the end of the incubation period. Person A aspirates off the transfection media, while person B adds 1.5 ml appropriate media to each well. Incubate at 37° C. for 45 or 72 hours depending on the media used: 1% BSA for 45 hours or CHO-5 for 72 hours.

On day four, using a 300 μl multichannel pipetter, aliquot 600 μl in one 1 ml deep well plate and the remaining supernatant into a 2 ml deep well. The supernatants from each well can then be used in the assays described in Examples 16-23.

It is specifically understood that when activity is obtained in any of the assays described below using a supernatant, the activity originates from either the METH1 or METH2 polypeptide directly (e.g., as a secreted protein) or by METH1 or METH2 inducing expression of other proteins, which are then secreted into the supernatant. Thus, the invention further provides a method of identifying the protein in the supernatant characterized by an activity in a particular assay.

### Example 15

### Construction of GAS Reporter Construct

One signal transduction pathway involved in the differentiation and proliferation of cells is called the Jaks-STATs pathway. Activated proteins in the Jaks-STATs pathway bind to gamma activation site “GAS” elements or interferon-sensitive responsive element (“ISRE”), located in the promoter of many genes. The binding of a protein to these elements alter the expression of the associated gene.

GAS and ISRE elements are recognized by a class of transcription factors called Signal Transducers and Activators of Transcription, or “STATs.” There are six members of the STATs family. Stat1 and Stat3 are present in many cell types, as is Stat2 (as response to IFN-alpha is widespread). Stat4 is more restricted and is not in many cell types though it has been found in T helper class I, cells after treatment with IL-12. Stat5 was originally called mammary growth factor, but has been found at higher concentrations in other cells including myeloid cells. It can be activated in tissue culture cells by many cytokines.

The STATs are activated to translocate from the cytoplasm to the nucleus upon tyrosine phosphorylation by a set of kinases known as the Janus Kinase (“Jaks”) family. Jaks represent a distinct family of soluble tyrosine kinases and include Tyk2, Jak1, Jak2, and Jak3. These kinases display significant sequence similarity and are generally catalytically inactive in resting cells.

The Jaks are activated by a wide range of receptors summarized in the Table below. (Adapted from review by Schidler and Darnell, Ann. Rev. Biochem. 64:621-51 (1995).) A cytokine receptor family, capable of activating Jaks, is divided into two groups: (a) Class 1 includes receptors for IL-2, IL-3, IL-4, IL-6, IL-7, IL-9, IL-11, IL-12, IL-15, Epo, PRL, GH, G-CSF, GM-CSF, LIF, CNTF, and thrombopoietin; and (b) Class 2 includes IFN-a, IFN-g, and IL-10. The Class 1 receptors share a conserved cysteine motif (a set of four conserved cysteines and one tryptophan) and a WSXWS motif (a membrane proxial region encoding Trp-Ser-Xxx-Trp-Ser (SEQ ID NO:82)).

Thus, on binding of a ligand to a receptor, Jaks are activated, which in turn activate STATs, which then translocate and bind to GAS elements. This entire process is encompassed in the Jaks-STATs signal transduction pathway.

Therefore, activation of the Jaks-STATs pathway, reflected by the binding of the GAS or the ISRE element, can be used to indicate proteins involved in the proliferation and differentiation of cells. For example, growth factors and cytokines are known to activate the Jaks-STATs pathway. (See Table below.) Thus, by using GAS elements linked to reporter molecules, activators of the Jaks-STATs pathway can be identified.

| | JAKs | | | | | |
|---|---|---|---|---|---|---|
| | tyk | Jak | Jak | Jak | | GAS (elements) |
| Ligand | 2 | 1 | 2 | 3 | STATS | or ISRE |
| IFN family | | | | | | |
| IFN-a/B | + | + | − | − | 1, 2, 3 | ISRE |
| IFN-g | | + | + | − | 1 | GAS (IRF1 > |
| Il-10 | + | ? | ? | − | 1, 3 | Lys6 > IFP) |
| gp130 family | | | | | | |
| IL-6 (Pleiotrophic) | + | + | + | ? | 1, 3 | GAS (IRF1 > |
| Il-11 (Pleiotrophic) | ? | + | ? | ? | 1, 3 | Lys6 > IFP) |
| OnM (Pleiotrophic) | ? | + | + | ? | 1, 3 | |
| LIF (Pleiotrophic) | ? | + | + | ? | 1, 3 | |
| CNTF (Pleiotrophic) | −/+ | + | + | ? | 1, 3 | |
| G-CSF (Pleiotrophic) | ? | + | ? | ? | 1, 3 | |
| IL-12 (Pleiotrophic) | + | − | + | + | 1, 3 | |
| g-C family | | | | | | |
| IL-2 (lymphocytes) | − | + | − | + | 1, 3, 5 | GAS |
| IL-4 | − | + | − | + | 6 | GAS (IRF1 = |
| (lymph/myeloid) | − | + | − | + | 5 | IFP >> Ly6)(IgH) |
| IL-7 (lymphocytes) | − | + | − | + | 5 | GAS |
| IL-9 (lymphocytes) | − | + | ? | ? | 6 | GAS |
| IL-13 (lymphocyte) | ? | + | ? | + | 5 | GAS |
| IL-15 | | | | | | GAS |
| gp140 family | | | | | | |
| IL-3 (myeloid) | − | − | + | − | 5 | GAS (IRF1 > |
| | | | | | | IFP >> Ly6) |
| IL-5 (myeloid) | − | − | + | − | 5 | GAS |
| GM-CSF (myeloid) | − | − | + | − | 5 | GAS |
| Growth hormone | | | | | | |
| family | | | | | | |
| GH | ? | − | + | − | 5 | GAS (B − CAS > |
| PRL | ? | +/− | + | − | 1, 3, 5 | IRF1 = IFP >> Ly6) |
| EPO | ? | − | + | − | 5 | |
| Receptor Tyrosine | | | | | | |
| Kinases | | | | | | |
| EGF | ? | + | + | − | 1, 3 | GAS (IRF1) |
| PDGF | ? | + | + | − | 1, 3 | GAS (not IRF1) |
| CSF-1 | ? | + | + | − | 1, 3 | |

To construct a synthetic GAS containing promoter element, which is used in the Biological Assays described in Examples 16-17, a PCR based strategy is employed to generate a GAS-SV40 promoter sequence. The 5′ primer contains four tandem copies of the GAS binding site found in the IRF1 promoter and previously demonstrated to bind STATs upon induction with a range of cytokines (Rothman et al., Immunity 1:457-468 (1994).), although other GAS or ISRE elements can be used instead. The 5′ primer also contains 18 bp of sequence complementary to the SV40 early promoter sequence and is flanked with an XhoI site. The sequence of the 5′ primer is:

(SEQ ID NO:86)

5′:GCGCCTCGAGATTTCCCCGAAATCTAGATTTCCCCGAAATGATTTCC
CCGAAATGATTTCCCCGAAATATCTGCCATCTCAATTAG:3′.

The downstream primer is complementary to the SV40 promoter and is flanked with a Hind III site: 5′:GCGGCAAGCTTTTTGCAAAGCCTAGGC:3′ (SEQ ID NO:87).

PCR amplification is performed using the SV40 promoter template present in the B-gal:promoter plasmid obtained from Clontech. The resulting PCR fragment is digested with XhoI/Hind III and subcloned into BLSK2-. (Stratagene.) Sequencing with forward and reverse primers confirms that the insert contains the following sequence:

(SEQ ID NO:88)

5′:CTCGAGATTTCCCCGAAATCTAGATTTCCCCGAAATGATTTCCCCGA
AATGATTTCCCCGAAATATCTGCCATCTCAATTAGTCAGCAACCATAGTC
CCGCCCCTAACTCCGCCCATCCCGCCCCTAACTCCGCCCAGTTCCGCCCA
TTCTCCGCCCCATGGCTGACTAATTTTTTTTATTTATGCAGAGGCCGAGG
CCGCCTCGGCCTCTGAGCTATTCCAGAAGTAGTGAGGAGGCTTTTTTGGA
GGCCTAGGCTTTTGCAAAAAGCTT:3′.

With this GAS promoter element linked to the SV40 promoter, a GAS:SEAP2 reporter construct is next engineered. Here, the reporter molecule is a secreted alkaline phosphatase, or “SEAP.” Clearly, however, any reporter molecule can used be instead of SEAP, in this or in any of the other Examples. Well known reporter molecules that can be used instead of SEAP include chloramphenicol acetyltransferase (CAT), luciferase, alkaline phosphatase, β-galactosidase, green fluorescent protein (GFP), or any protein detectable by an antibody.

The above sequence confirmed synthetic GAS-SV40 promoter element is subcloned into the pSEAP-Promoter vector obtained from Clontech using HindIII and XhoI, effectively replacing the SV40 promoter with the amplified GAS:SV40 promoter element, to create the GAS-SEAP vector. However, this vector does not contain a neomycin resistance gene, and therefore, is not preferred for mammalian expression systems.

Thus, in order to generate mammalian stable cell lines expressing the GAS-SEAP reporter, the GAS-SEAP cassette is removed from the GAS-SEAP vector using SalI and NotI, and inserted into a backbone vector containing the neomycin resistance gene, such as pGFP-1 (Clontech), using these restriction sites in the multiple cloning site, to create the GAS-SEAP/Neo vector. Once this vector is transfected into mammalian cells, this vector can then be used as a reporter molecule for GAS binding as described in Examples 16-17.

Other constructs can be made using the above description and replacing GAS with a different promoter sequence. For example, construction of reporter molecules containing NFK-B and EGR promoter sequences are described in Examples 18 and 19. However, many other promoters can be substituted using the protocols described in these Examples. For instance, SRE, IL-2, NFAT, or Osteocalcin promoters can be substituted, alone or in combination (e.g., GAS/NF-KB/EGR, GAS/NF-KB, 11-2/NFAT, or NF-KB/GAS). Similarly, other cell lines can be used to test reporter construct activity, such as HELA (epithelial), HUVEC (endothelial), Reh (B-cell), Saos-2 (osteoblast), HUVAC (aortic), or Cardiomyocyte.

### Example 16

### High-Throughput Screening Assay for T-cell Activity

The following protocol is used to assess T-cell activity of METH1 or METH2 by determining whether METH1 or METH2 supernatant proliferates and/or differentiates T-cells. T-cell activity is assessed using the GAS/SEAP/Neo construct produced in Example 15. Thus, factors that increase SEAP activity indicate the ability to activate the Jaks-STATS signal transduction pathway. The T-cell used in this assay is Jurkat T-cells (ATCC Accession No. TIB-152), although Molt-3 cells (ATCC Accession No. CRL-1552) and Molt-4 cells (ATCC Accession No. CRL-1582) cells can also be used.

Jurkat T-cells are lymphoblastic CD4⁺ Th1 helper cells. In order to generate stable cell lines, approximately 2 million Jurkat cells are transfected with the GAS-SEAP/neo vector using DMRIE-C (Life Technologies)(transfection procedure described below). The transfected cells are seeded to a density of approximately 20,000 cells per well and transfectants resistant to 1 mg/ml genticin selected. Resistant colonies are expanded and then tested for their response to increasing concentrations of interferon gamma. The dose response of a selected clone is demonstrated.

Specifically, the following protocol will yield sufficient cells for 75 wells containing 200 μl of cells. Thus, it is either scaled up, or performed in multiple to generate sufficient cells for multiple 96 well plates. Jurkat cells are maintained in RPMI+10% serum with 1% Pen-Strep. Combine 2.5 mls of OPTI-MEM (Life Technologies) with 10 ug of plasmid DNA in a T25 flask. Add 2.5 ml OPTI-MEM containing 50 μl of DMRIE-C and incubate at room temperature for 1545 mins.

During the incubation period, count cell concentration, spin down the required number of cells (10⁷ per transfection), and resuspend in OPTI-MEM to a final concentration of 10⁷ cells/ml. Then add 1 ml of 1×10⁷ cells in OPTI-MEM to T25 flask and incubate at 37° C. for 6 hrs. After the incubation, add 10 ml of RPMI+15% serum.

The Jurkat:GAS-SEAP stable reporter lines are maintained in RPMI+10% serum, 1 mg/ml Genticin, and 1% Pen-Strep. These cells are treated with supernatants containing METH1 or METH2 polypeptides or METH1 or METH2 induced polypeptides as produced by the protocol described in Example 14.

On the day of treatment with the supernatant, the cells should be washed and resuspended in fresh RPMI+10% serum to a density of 500,000 cells per ml. The exact number of cells required will depend on the number of supernatants being screened. For one 96 well plate, approximately 10 million cells (for 10 plates, 100 million cells) are required.

Transfer the cells to a triangular reservoir boat, in order to dispense the cells into a 96 well dish, using a 12 channel pipette. Using a 12 channel pipette, transfer 200 μl of cells into each well (therefore adding 100,000 cells per well).

After all the plates have been seeded, 50 μl of the supernatants are transferred directly from the 96 well plate containing the supernatants into each well using a 12 channel pipette. In addition, a dose of exogenous interferon gamma (0.1, 1.0, 10 ng) is added to wells H9, H10, and H11 to serve as additional positive controls for the assay.

The 96 well dishes containing Jurkat cells treated with supernatants are placed in an incubator for 48 hrs (note: this time is variable between 48-72 hrs). 35 μl samples from each well are then transferred to an opaque 96 well plate using a 12 channel pipette. The opaque plates should be covered (using sellophene covers) and stored at −20° C. until SEAP assays are performed according to Example 20. The plates containing the remaining treated cells are placed at 4° C. and serve as a source of material for repeating the assay on a specific well if desired.

As a positive control, 100 Unit/ml interferon gamma can be used which is known to activate Jurkat T cells. Over 30 fold induction is typically observed in the positive control wells.

### Example 17

### High-Throughput Screening Assay Identifying Myeloid Activity

The following protocol is used to assess myeloid activity of METH1 or METH2 by determining whether METH1 or METH2 proliferates and/or differentiates myeloid cells. Myeloid cell activity is assessed using the GAS/SEAP/Neo construct produced in Example 15. Thus, factors that increase SEAP activity indicate the ability to activate the Jaks-STATS signal transduction pathway. The myeloid cell used in this assay is U937, a pre-monocyte cell line, although TF-1, HL60, or KG1 can be used.

To transiently transfect U937 cells with the GAS/SEAP/Neo construct produced in Example 15, a DEAE-Dextran method (Kharbanda et. al., 1994, Cell Growth & Differentiation 5:259-265) is used. First, harvest 2×10e⁷ U937 cells and wash with PBS. The U937 cells are usually grown in RPMI 1640 medium containing 10% heat-inactivated fetal bovine serum (FBS) supplemented with 100 units/ml penicillin and 100 mg/ml streptomycin.

Next, suspend the cells in 1 ml of 20 mM Tris-HCl (pH 7.4) buffer containing 0.5 mg/ml DEAE-Dextran, 8 ug GAS-SEAP2 plasmid DNA, 140 mM NaCl, 5 mM KCl, 375 uM Na₂HPO₄.7H₂O, 1 MM MgCl₂, and 675 uM CaCl₂. Incubate at 37° C. for 45 min.

Wash the cells with RPMI 1640 medium containing 10% FBS and then resuspend in 10 ml complete medium and incubate at 37° C. for 36 hr.

The GAS-SEAP/U937 stable cells are obtained by growing the cells in 400 ug/ml G418. The G418-free medium is used for routine growth but every one to two months, the cells should be re-grown in 400 ug/ml G418 for couple of passages.

These cells are tested by harvesting 1×10⁸ cells (this is enough for ten 96-well plates assay) and wash with PBS. Suspend the cells in 200 ml above described growth medium, with a final density of 5×10⁵ cells/ml. Plate 200 μl cells per well in the 96-well plate (or 1×10⁵ cells/well).

Add 50 μl of the supernatant prepared by the protocol described in Example 14. Incubate at 37° C. for 48 to 72 hr. As a positive control, 100 Unit/ml interferon gamma can be used which is known to activate U937 cells. Over 30 fold induction is typically observed in the positive control wells. SEAP assay the supernatant according to the protocol described in Example 20.

### Example 18

### High-Throughput Screening Assay Identifying Neuronal Activity

When cells undergo differentiation and proliferation, a group of genes are activated through many different signal transduction pathways. One of these genes, EGR1 (early growth response gene 1), is induced in various tissues and cell types upon activation. The promoter of EGR1 is responsible for such induction. Using the EGR1 promoter linked to reporter molecules, activation of cells can be assessed by METH1 or METH2.

Particularly, the following protocol is used to assess neuronal activity in PC12 cell lines. PC12 cells (rat phenochromocytoma cells) are known to proliferate and/or differentiate by activation with a number of mitogens, such as TPA (tetradecanoyl phorbol acetate), NGF (nerve growth factor), and EGF (epidermal growth factor). The EGR1 gene expression is activated during this treatment. Thus, by stably transfecting PC12 cells with a construct containing an EGR promoter linked to SEAP reporter, activation of PC12 cells by METH1 or METH2 can be assessed.

The EGR/SEAP reporter construct can be assembled by the following protocol. The EGR-1 promoter sequence (-633 to +1)(Sakamoto K et al., Oncogene 6:867-871 (1991)) can be PCR amplified from human genomic DNA using the following primers:

(SEQ ID NO:89)

5′GCGCTCGAGGGATGACAGCGATAGAACCCCGG-3′

(SEQ ID NO:90)

5′GCGAAGCTTCGCGACTCCCCGGATCCGCCTC-3′.

Using the GAS:SEAP/Neo vector produced in Example 15, EGR1 amplified product can then be inserted into this vector. Linearize the GAS:SEAP/Neo vector using restriction enzymes XhoI/HindIII, removing the GAS/SV40 stuffer. Restrict the EGR1 amplified product with these same enzymes. Ligate the vector and the EGR1 promoter.

To prepare 96 well-plates for cell culture, two mls of a coating solution (1:30 dilution of collagen type I (Upstate Biotech Inc. Cat#08-115) in 30% ethanol (filter sterilized)) is added per one 10 cm plate or 50 ml per well of the 96-well plate, and allowed to air dry for 2 hr.

PC12 cells are routinely grown in RPMI-1640 medium (Bio Whittaker) containing 10% horse serum (JRH BIOSCIENCES, Cat. # 12449-78P), 5% heat-inactivated fetal bovine serum (FBS) supplemented with 100 units/ml penicillin and 100 ug/ml streptomycin on a precoated 10 cm tissue culture dish. One to four split is done every three to four days. Cells are removed from the plates by scraping and resuspended with pipetting up and down for more than 15 times.

Transfect the EGR/SEAP/Neo construct into PC12 using the Lipofectamine protocol described in Example 14. EGR-SEAP/PC12 stable cells are obtained by growing the cells in 300 ug/ml G418. The G418-free medium is used for routine growth but every one to two months, the cells should be re-grown in 300 ug/mil G418 for couple of passages.

To assay for neuronal activity, a 10 cm plate with cells around 70 to 80% confluent is screened by removing the old medium. Wash the cells once with PBS (Phosphate buffered saline). Then starve the cells in low serum medium (RPMI-1640 containing 1% horse serum and 0.5% FBS with antibiotics) overnight.

The next morning, remove the medium and wash the cells with PBS. Scrape off the cells from the plate, suspend the cells well in 2 ml low serum medium. Count the cell number and add more low serum medium to reach final cell density as 5×10⁵ cells/ml.

Add 200 μl of the cell suspension to each well of 96-well plate (equivalent to 1×10⁵ cells/well). Add 50 μl supernatant produced by Example 14,37° C. for 48 to 72 hr. As a positive control, a growth factor known to activate PC12 cells through EGR can be used, such as 50 ng/ul of Neuronal Growth Factor (NGF). Over fifty-fold induction of SEAP is typically seen in the positive control wells. SEAP assay the supernatant according to Example 20.

### Example 19

### High-Throughput Screening Assay for T-cell Activity

NF-KB (Nuclear Factor KB) is a transcription factor activated by a wide variety of agents including the inflammatory cytokines IL-1 and TNF, CD30 and CD40, lymphotoxin-alpha and lymphotoxin-beta, by exposure to LPS or thrombin, and by expression of certain viral gene products. As a transcription factor, NF-KB regulates the expression of genes involved in immune cell activation, control of apoptosis (NF-B appears to shield cells from apoptosis), B and T-cell development, anti-viral and antimicrobial responses, and multiple stress responses. In non-stimulated conditions, NF-KB is retained in the cytoplasm with I-KB (Inhibitor KB). However, upon stimulation, I-KB is phosphorylated and degraded, causing NF-KB to shuttle to the nucleus, thereby activating transcription of target genes. Target genes activated by NF-KB include IL-2, IL-5 μL-6, GM-CSF, ICAM-1 and class 1 MHC.

Due to its central role and ability to respond to a range of stimuli, reporter constructs utilizing the NF-KB promoter element are used to screen the supernatants produced in Example 14. Activators or inhibitors of NF-KB would be useful in treating diseases. For example, inhibitors of NF-KB could be used to treat those diseases related to the acute or chronic activation of NF-KB, such as rheumatoid arthritis.

To construct a vector containing the NF-KB promoter element, a PCR based strategy is employed. The upstream primer contains four tandem copies of the NF-KB binding site (GGGGACTTTCCC) (SEQ ID NO:91), 18 bp of sequence complementary to the 5′ end of the SV40 early promoter sequence, and is flanked with an XhoI site:

(SEQ ID NO:92)

5′: GCGGCCTCGAGGGGACTTTCCCGGGGACTTTCCGGGGACTTTCCGGG
ACTTTCCATCCTGCCATCTCAATTAG:3′.

The downstream primer is complementary to the 3′ end of the SV40 promoter and is flanked with a Hind III site:

5′:GCGGCAAGCTTTTTGCAAAGCCTAGGC:3′ (SEQ ID NO:93).

PCR amplification is performed using the SV40 promoter template present in the pB-gal:promoter plasmid obtained from Clontech. The resulting PCR fragment is digested with XhoI and Hind III and subcloned into BLSK2-. (Stratagene) Sequencing with the T7 and T3 primers confirms the insert contains the following sequence:

(SEQ ID NO:88)

5′: CTCGAGGGGACTTTCCCGGGGACTTTCCGGGGACTTTCCGGGACTTT
CCATCTGCCATCTCAATTAGTCAGCAACCATAGTCCCGCCCCTAACTCCG
CCCATCCCGCCCCTAACTCCGCCCAGTTCCGCCCATTCTCCGCCCCATGG
CTGACTAATTTTTTTTATTTATGCAGAGGCCGAGGCCGCCTCGGCCTCTG
AGCTATTCCAGAAGTAGTGAGGAGGCTTTTTTGGAGGCCTAGGCTTTTGC
AAAAAGCTT:3′

Next, replace the SV40 minimal promoter element present in the pSEAP2-promoter plasmid (Clontech) with this NF-KB/SV40 fragment using XhoI and HindIII. However, this vector does not contain a neomycin resistance gene, and therefore, is not preferred for mammalian expression systems.

In order to generate stable mammalian cell lines, the NF-KB/SV40/SEAP cassette is removed from the above NF-KB/SEAP vector using restriction enzymes SalI and NotI, and inserted into a vector containing neomycin resistance. Particularly, the NF-KB/SV40/SEAP cassette was inserted into pGFP-1 (Clontech), replacing the GFP gene, after restricting pGFP-1 with SalI and NotI.

Once NF-KB/SV40/SEAP/Neo vector is created, stable Jurkat T-cells are created and maintained according to the protocol described in Example 16. Similarly, the method for assaying supernatants with these stable Jurkat T-cells is also described in Example 16. As a positive control, exogenous TNF alpha (0.1,1, 10 ng) is added to wells H9, H10, and HI 1, with a 5-10 fold activation typically observed.

### Example 20

### Assay for SEAP Activity

As a reporter molecule for the assays described in Examples 16-19, SEAP activity is assayed using the Tropix Phospho-light Kit (Cat. BP-400) according to the following general procedure. The Tropix Phospho-light Kit supplies the Dilution, Assay, and Reaction Buffers used below.

Prime a dispenser with the 2.5× Dilution Buffer and dispense 15 μl of 2.5×dilution buffer into Optiplates containing 35 ul of a supernatant. Seal the plates with a plastic sealer and incubate at 65° C. for 30 min. Separate the Optiplates to avoid uneven heating.

Cool the samples to room temperature for 15 minutes. Empty the dispenser and prime with the Assay Buffer. Add 50 ml Assay Buffer and incubate at room temperature 5 min. Empty the dispenser and prime with the Reaction Buffer (see the table below). Add 50 μl Reaction Buffer and incubate at room temperature for 20 minutes. Since the intensity of the chemiluminescent signal is time dependent, and it takes about 10 minutes to read 5 plates on luminometer, one should treat 5 plates at each time and start the second set 10 minutes later.

Read the relative light unit in the luminometer. Set H12 as blank, and print the results. An increase in chemiluminescence indicates reporter activity.

Reaction Buffer Formulation:

| # of plates | Rxn buffer diluent (ml) | CSPD (ml) |
|---|---|---|
| 10 | 60 | 3 |
| 11 | 65 | 3.25 |
| 12 | 70 | 3.5 |
| 13 | 75 | 3.75 |
| 14 | 80 | 4 |
| 15 | 85 | 4.25 |
| 16 | 90 | 4.5 |
| 17 | 95 | 4.75 |
| 18 | 100 | 5 |
| 19 | 105 | 5.25 |
| 20 | 110 | 5.5 |
| 21 | 115 | 5.75 |
| 22 | 120 | 6 |
| 23 | 125 | 6.25 |
| 24 | 130 | 6.5 |
| 25 | 135 | 6.75 |
| 26 | 140 | 7 |
| 27 | 145 | 7.25 |
| 28 | 150 | 7.5 |
| 29 | 155 | 7.75 |
| 30 | 160 | 8 |
| 31 | 165 | 8.25 |
| 32 | 170 | 8.5 |
| 33 | 175 | 8.75 |
| 34 | 180 | 9 |
| 35 | 185 | 9.25 |
| 36 | 190 | 9.5 |
| 37 | 195 | 9.75 |
| 38 | 200 | 10 |
| 39 | 205 | 10.25 |
| 40 | 210 | 10.5 |
| 41 | 215 | 10.75 |
| 42 | 220 | 11 |
| 43 | 225 | 11.25 |
| 44 | 230 | 11.5 |
| 45 | 235 | 11.75 |
| 46 | 240 | 12 |
| 47 | 245 | 12.25 |
| 48 | 250 | 12.5 |
| 49 | 255 | 12.75 |
| 50 | 260 | 13 |

Example 21: High-Throughput Screening Assay Identifying Changes in Small Molecule Concentration and Membrane Permeability

Binding of a ligand to a receptor is known to alter intracellular levels of small molecules, such as calcium, potassium, sodium, and pH, as well as alter membrane potential. These alterations can be measured in an assay to identify supernatants which bind to receptors of a particular cell. Although the following protocol describes an assay for calcium, this protocol can easily be modified to detect changes in potassium, sodium, pH, membrane potential, or any other small molecule which is detectable by a fluorescent probe.

The following assay uses Fluorometric Imaging Plate Reader (“FLIPR”) to measure changes in fluorescent molecules (Molecular Probes) that bind small molecules. Clearly, any fluorescent molecule detecting a small molecule can be used instead of the calcium fluorescent molecule, fluo-3, used here.

For adherent cells, seed the cells at 10,000-20,000 cells/well in a Co-star black 96-well plate with clear bottom. The plate is incubated in a CO₂ incubator for 20 hours. The adherent cells are washed two times in Biotek washer with 200 μl of HBSS (Hank's Balanced Salt Solution) leaving 100 μl of buffer after the final wash.

A stock solution of 1 mg/ml fluo-3 is made in 10% pluronic acid DMSO. To load the cells with fluo-3, 50 μl of 12 ug/ml fluo-3 is added to each well. The plate is incubated at 37° C. in a CO₂ incubator for 60 min. The plate is washed four times in the Biotek washer with BSS leaving 100 μl of buffer.

For non-adherent cells, the cells are spun down from culture media. Cells are re-suspended to 2-5×10⁶ cells/ml with 1BSS in a 50-ml conical tube. 4 μl of 1 mg/ml fluo-3 solution in 10% pluronic acid DMSO is added to each ml of cell suspension. The tube is then placed in a 37° C. water bath for 30-60 min. The cells are washed twice with HBSS, resuspended to 1×10⁶ cells/ml, and dispensed into a microplate, 100 μl/well. The plate is centrifuged at 1000 rpm for 5 min. The plate is then washed once in Denley CellWash with 200 μl, followed by an aspiration step to 100 μl final volume.

For a non-cell based assay, each well contains a fluorescent molecule, such as fluo-3. The supernatant is added to the well, and a change in fluorescence is detected.

To measure the fluorescence of intracellular calcium, the FLIPR is set for the following parameters: (1) System gain is 300-800 mW; (2) Exposure time is 0.4 second; (3) Camera F/stop is F/2; (4) Excitation is 488 nm; (5) Emission is 530 nm; and (6) Sample addition is 50 μl. Increased emission at 530 nm indicates an extracellular signaling event caused by the a molecule, either METH1 or METH2 or a molecule induced by METH1 or METH2, which has resulted in an increase in the intracellular Ca⁺⁺ concentration.

### Example 22

### High-Throughput Screening Assay Identifying Tyrosine Kinase Activity

The Protein Tyrosine Kinases (PTK) represent a diverse group of transmembrane and cytoplasmic kinases. Within the Receptor Protein Tyrosine Kinase RPTK) group are receptors for a range of mitogenic and metabolic growth factors including the PDGF, FGF, EGF, NGF, HGF and Insulin receptor subfamilies. In addition there are a large family of RPTKs for which the corresponding ligand is unknown. Ligands for RPTKs include mainly secreted small proteins, but also membrane-bound and extracellular matrix proteins.

Activation of RPTK by ligands involves ligand-mediated receptor dimerization, resulting in transphosphorylation of the receptor subunits and activation of the cytoplasmic tyrosine kinases. The cytoplasmic tyrosine kinases include receptor associated tyrosine kinases of the src-family (e.g., src, yes, lck, lyn, fyn) and non-receptor linked and cytosolic protein tyrosine kinases, such as the Jak family, members of which mediate signal transduction triggered by the cytokine superfamily of receptors (e.g., the Interleukins, Interferons, GM-CSF, and Leptin).

Because of the wide range of known factors capable of stimulating tyrosine kinase activity, identifying whether METH1 or METH2 or a molecule induced by METH1 or METH2 is capable of activating tyrosine kinase signal transduction pathways is of interest. Therefore, the following protocol is designed to identify such molecules capable of activating the tyrosine kinase signal transduction pathways.

Seed target cells (e.g., primary keratinocytes) at a density of approximately 25,000 cells per well in a 96 well Loprodyne Silent Screen Plates purchased from Nalge Nunc (Naperville, Ill.). The plates are sterilized with two 30 minute rinses with 100% ethanol, rinsed with water and dried overnight. Some plates are coated for 2 hr with 100 ml of cell culture grade type I collagen (50 mg/ml), gelatin (2%) or polylysine (50 mg/ml), all of which can be purchased from Sigma Chemicals (St. Louis, Mo.) or 10% Matrigel purchased from Becton Dickinson (Bedford, Mass.), or calf serum, rinsed with PBS and stored at 4° C. Cell growth on these plates is assayed by seeding 5,000 cells/well in growth medium and indirect quantitation of cell number through use of alamarBlue as described by the manufacturer Alamar Biosciences, Inc. (Sacramento, Calif.) after 48 hr. Falcon plate covers #3071 from Becton Dickinson (Bedford, Mass.) are used to cover the Loprodyne Silent Screen Plates. Falcon Microtest III cell culture plates can also be used in some proliferation experiments.

To prepare extracts, A431 cells are seeded onto the nylon membranes of Loprodyne plates (20,000/200 ml/well) and cultured overnight in complete medium. Cells are quiesced by incubation in serum-free basal medium for 24 hr. After 5-20 minutes treatment with EGF (60 ng/ml) or 50 μl of the supernatant produced in Example 14, the medium was removed and 100 ml of extraction buffer ((20 mM HEPES pH 7.5, 0.15 M NaCl, 1% Triton X-100, 0.1% SDS, 2 mM Na3VO4, 2 mM Na4P2O7 and a cocktail of protease inhibitors (#1836170) obtained from Boehringer Mannheim (Indianapolis, Ind.) is added to each well and the plate is shaken on a rotating shaker for 5 minutes at 4° C. The plate is then placed in a vacuum transfer manifold and the extract filtered through the 0.45 mm membrane bottoms of each well using house vacuum. Extracts are collected in a 96-well catch/assay plate in the bottom of the vacuum manifold and immediately placed on ice. To obtain extracts clarified by centrifugation, the content of each well, after detergent solubilization for 5 minutes, is removed and centrifuged for 15 minutes at 4° C. at 16,000× g.

Test the filtered extracts for levels of tyrosine kinase activity. Although many methods of detecting tyrosine kinase activity are known, one method is described here.

Generally, the tyrosine kinase activity of a supernatant is evaluated by determining its ability to phosphorylate a tyrosine residue on a specific substrate (a biotinylated peptide). Biotinylated peptides that can be used for this purpose include PSK1 (corresponding to amino acids 6-20 of the cell division kinase cdc2-p34) and PSK2 (corresponding to amino acids 1-17 of gastrin). Both peptides are substrates for a range of tyrosine kinases and are available from Boehringer Mannheim.

The tyrosine kinase reaction is set up by adding the following components in order. First, add 10 ul of 5 uM Biotinylated Peptide, then 10 ul ATP/Mg²+(5 mM ATP/50 mM MgCl2), then 10 ul of 5× Assay Buffer (40 mM imidazole hydrochloride, pH 7.3, 40 mM beta-glycerophosphate, 1 mM EGTA, 100 mM MgCl₂, 5 mM MnCl₂, 0.5 mg/ml BSA), then 5 ul of Sodium Vanadate(1 mM), and then 5 ul of water. Mix the components gently and preincubate the reaction mix at 30° C. for 2 min. Initial the reaction by adding 10 ul of the control enzyme or the filtered supernatant.

The tyrosine kinase assay reaction is then terminated by adding 10 μl of 120 mm EDTA and place the reactions on ice.

Tyrosine kinase activity is determined by transferring 50 μl aliquot of reaction mixture to a microtiter plate (MTP) module and incubating at 37° C. for 20 min. This allows the streptavadin coated 96 well plate to associate with the biotinylated peptide. Wash the MTP module with 300 ul/well of PBS four times. Next add 75 μl of anti-phospotyrosine antibody conjugated to horse radish peroxidase(anti-P-Tyr-POD (0.5u/ml)) to each well and incubate at 37° C. for one hour. Wash the well as above.

Next add 100 ul of peroxidase substrate solution (Boehringer Mannheim) and incubate at room temperature for at least 5 mins (up to 30 min). Measure the absorbance of the sample at 405 nm by using ELISA reader. The level of bound peroxidase activity is quantitated using an ELISA reader and reflects the level of tyrosine kinase activity.

### Example 23

### High-Throughput Screening Assay Identifying Phosphorylation Activity

As a potential alternative and/or compliment to the assay of protein tyrosine kinase activity described in Example 22, an assay which detects activation (phosphorylation) of major intracellular signal transduction intermediates can also be used. For example, as described below one particular assay can detect tyrosine phosphorylation of the Erk-1 and Erk-2 kinases. However, phosphorylation of other molecules, such as Raf, JNK, p38 MAP, Map kinase kinase (MEK), MEK kinase, Src, Muscle specific kinase (MuSK), IRAK, Tec, and Janus, as well as any other phosphoserine, phosphotyrosine, or phosphothreonine molecule, can be detected by substituting these molecules for Erk-1 or Erk-2 in the following assay.

Specifically, assay plates are made by coating the wells of a 96-well ELISA plate with 0.1 ml of protein G (1 ug/ml) for 2 hr at room temp, (RT). The plates are then rinsed with PBS and blocked with 3% BSA/PBS for 1 hr at RT. The protein G plates are then treated with 2 commercial monoclonal antibodies (10 ng/well) against Erk-1 and Erk-2 (1 hr at RT) (Santa Cruz Biotechnology). (To detect other molecules, this step can easily be modified by substituting a monoclonal antibody detecting any of the above described molecules.) After 3-5 rinses with PBS, the plates are stored at 4° C. until use.

A431 cells are seeded at 20,000/well in a 96-well Loprodyne filterplate and cultured overnight in growth medium. The cells are then starved for 48 hr in basal medium (DMEM) and then treated with EGF (6 ng/well) or 50 μl of the supernatants obtained in Example 14 for 5-20 minutes. The cells are then solubilized and extracts filtered directly into the assay plate.

After incubation with the extract for 1 hr at RT, the wells are again rinsed. As a positive control, a commercial preparation of MAP kinase (10 ng/well) is used in place of A431 extract. Plates are then treated with a commercial polyclonal (rabbit) antibody (1 ug/ml) which specifically recognizes the phosphorylated epitope of the Erk-1 and Erk-2 kinases (1 hr at RT). This antibody is biotinylated by standard procedures. The bound polyclonal antibody is then quantitated by successive incubations with Europium-streptavidin and Europium fluorescence enhancing reagent in the Wallac DELFIA instrument (time-resolved fluorescence). An increased fluorescent signal over background indicates a phosphorylation by METH1 or METH2 or a molecule induced by METH1 or METH2.

### Example 24

### Method of Determining Alterations in the METH1 or METH2 Gene

RNA isolated from entire families or individual patients presenting with a phenotype of interest (such as a disease) is be isolated. cDNA is then generated from these RNA samples using protocols known in the art. (See, Sambrook.) The cDNA is then used as a template for PCR, employing primers surrounding regions of interest in SEQ ID NO:1. Suggested PCR conditions consist of 35 cycles at 95° C. for 30 seconds; 60-120 seconds at 52-58° C.; and 60-120 seconds at 70° C., using buffer solutions described in Sidransky, D. et al., Science 252:706 (1991).

PCR products are then sequenced using primers labeled at their 5′ end with T4 polynucleotide kinase, employing SequiTherm Polymerase. (Epicentre Technologies). The intron-exon borders of selected exons of METH1 or METH2 is also determined and genomic PCR products analyzed to confirm the results. PCR products harboring suspected mutations in METH1 or METH2 is then cloned and sequenced to validate the results of the direct sequencing.

PCR products of METH1 or METH2 are cloned into T-tailed vectors as described in Holton, T. A. and Graham, M. W., Nucleic Acids Research 19:1156 (1991) and sequenced with T7 polymerase (United States Biochemical). Affected individuals are identified by mutations in METH1 or METH2 not present in unaffected individuals.

Genomic rearrangements are also observed as a method of determining alterations in the METH1 or METH2 gene. Isolated genomic clones are nick-translated with digoxigenindeoxy-uridine 5′-triphosphate (Boehringer Manheim), and FISH performed as described in Johnson, Cg. et al., Methods Cell Biol. 35:73-99 (1991). Hybridization with the labeled probe is carried out using a vast excess of human cot-1 DNA for specific hybridization to the METH1 or METH2 genomic locus.

Chromosomes are counterstained with 4,6-diamino-2-phenylidole and propidium iodide, producing a combination of C— and R-bands. Aligned images for precise mapping are obtained using a triple-band filter set (Chroma Technology, Brattleboro, Vt.) in combination with a cooled charge-coupled device camera (Photometrics, Tucson, Ariz.) and variable excitation wavelength filters. (Johnson, Cv. et al., Genet. Anal. Tech. Appl. 8:75 (1991).) Image collection, analysis and chromosomal fractional length measurements are performed using the ISee Graphical Program System. (Inovision Corporation, Durham, N.C.) Chromosome alterations of the genomic region of METH1 or METH2 (hybridized by the probe) are identified as insertions, deletions, and translocations. These METH1 or METH2 alterations are used as a diagnostic marker for an associated disease.

### Example 25

### Method of Detecting Abnormal Levels of METH1 or METH2 in a Biological Sample

METH1 or METH2 polypeptides can be detected in a biological sample, and if an increased or decreased level of METH1 or METH2 is detected, this polypeptide is a marker for a particular phenotype. Methods of detection are numerous, and thus, it is understood that one skilled in the art can modify the following assay to fit their particular needs.

For example, antibody-sandwich ELISAs are used to detect METH1 or METH2 in a sample, preferably a biological sample. Wells of a microtiter plate are coated with specific antibodies to METH1 or METH2, at a final concentration of 0.2 to 10 ug/ml. The antibodies are either monoclonal or polyclonal and are produced by the method described in Example 13. The wells are blocked so that non-specific binding of METH1 or METH2 to the well is reduced.

The coated wells are then incubated for >2 hours at RT with a sample containing METH1 or METH2. Preferably, serial dilutions of the sample should be used to validate results. The plates are then washed three times with deionized or distilled water to remove unbounded METH1 or METH2.

Next, 50 μl of specific antibody-alkaline phosphatase conjugate, at a concentration of 25-400 ng, is added and incubated for 2 hours at room temperature. The plates are again washed three times with deionized or distilled water to remove unbounded conjugate.

Add 75 μl of 4-methylumbelliferyl phosphate (MUP) or p-nitrophenyl phosphate (NPP) substrate solution to each well and incubate 1 hour at room temperature. Measure the reaction by a microtiter-plate reader. Prepare a standard curve, using serial dilutions of a control sample, and plot METH1 or METH2 polypeptide concentration on the X-axis (log scale) and fluorescence or absorbance of the Y-axis (linear scale). Interpolate the concentration of the METH1 or METH2 in the sample using the standard curve.

### Example 26

### Formulating a Polypeptide

The METH1 or METH2 composition will be formulated and dosed in a fashion consistent with good medical practice, taking into account the clinical condition of the individual patient (especially the side effects of treatment with the METH1 or METH2 polypeptide alone), the site of delivery, the method of administration, the scheduling of administration, and other factors known to practitioners. The “effective amount” for purposes herein is thus determined by such considerations.

As a general proposition, the total pharmaceutically effective amount of METH1 or METH2 administered parenterally per dose will be in the range of about 1 ug/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, METH1 or METH2 is typically administered at a dose rate of about 1 ug/kg/hour to about 50 ug/kg/hour, either by 14 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing METH1 or METH2 are administered orally, rectally, parenterally, intracistemally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any type. The term “parenteral” as used herein refers to modes of administration which include intravenous, intramuscular, intraperitoneal, intrastemal, subcutaneous and intraarticular injection and infusion.

METH1 or METH2 is also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al.) or poly-D-(−)-3-hydroxybutyric acid (EP 133,988). Sustained-release compositions also include liposomally entrapped METH1 or METH2 polypeptides. Liposomes containing the METH1 or METH2 are prepared by methods known per se: DE 3,218,121; Epstein et al., Proc. Natl. Acad. Sci. USA 82:3688-3692 (1985); Hwang et al., Proc. Natl. Acad. Sci. USA 77:40304034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal secreted polypeptide therapy.

For parenteral administration, in one embodiment, METH1 or METH2 is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting METH1 or METH2 uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

METH1 or METH2 is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of polypeptide salts.

METH1 or METH2 used for therapeutic administration can be sterile. Sterility is readily accomplished by filtration through sterile filtration membranes (e.g., 0.2 micron membranes). Therapeutic polypeptide compositions generally are placed into a container having a sterile access port, for example, an intravenous solution bag or vial having a stopper pierceable by a hypodermic injection needle.

METH1 or METH2 polypeptides ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous METH1 or METH2 polypeptide solution, and the resulting mixture is lyophilized. The infusion solution is prepared by reconstituting the lyophilized METH1 or METH-2 polypeptide using bacteriostatic Water-for-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, METH1 or METH2 may be employed in conjunction with other therapeutic compounds.

The compositions of the invention may be administered alone or in combination with other therapeutic agents. Therapeutic agents that may be administered in combination with the compositions of the invention include, but are not limited to, other members of the TNF family, chemotherapeutic agents, antibiotic, steroidal and nonsteroidal anti-inflammatories, conventional immunotherapeutic agents, cytokines and/or growth factors. Combination may be administered either concomitantly, e.g. as an admixture; separately but simultaneously or concurrently; or sequentially. This includes presentations in which the combined agents are administered together as a therapeutic mixture, and also procedures in which the combined agents are administered separately but simultaneously, e.g. as through separate intravenous lines into the same individual. Administration “in combination” further includes the separate administration of one of the compounds or agents given first, followed by the second.

In one embodiment, the compositions of the invention are administered in combination with other members of the TNF family. TNF, TNF-related or TNF-like molecules that may be administered with the compositions of the invention include, but are not limited to, soluble forms of TNF-alpha, lymphotoxin alpha (LT-alpha, also known as TNF-beta), LT-beta (found in complex heterotrimer LT-alph2-beta), OPGL, FasL, CD27L, CD30L, CD40L, 4-1BBL, DcR3, OX40L, TNF-gamma (International Publication No. WO 96/14328), AIM-I (International Publication No. WO 97/33899), endokine-alpha (International Publication No. WO 98/07880), TR6 (International Publication No. WO 98/30694), OPG, and neutrokine-alpha (International Publication No. WO 98/18921), OX40, and nerve growth factor (NGF) and soluble forms of Fas, CD30, CD27, CD40 and 4-IBB, TR2 (International Publication No. WO 98/34095), DR3 (International Publication No. WO 97/33904), DR4 (International Publication No. WO 98 /32856), TR5 (International Publication No. WO 98/30693), TR6 (International Publication No. WO 98/30694), TR7 (International Publication No. WO 98/41629), TRANK, TR9 (International Publication No. WO 98/56892), TR10 (International Publication No. WO 98/54202), 312C2 (International Publication No. WO 98106842), and TR12, and soluble forms of CD154, CD70 and CD153.

Conventional nonspecific immunosuppressive agents that may be administered in combination with the compositions of the invention include, but are not limited to, steroids, cyclosporine, cyclosporine analogs, ayclophosphamide methylprednisone, prednisone, azathioprine, FK-506, 15-deoxyspergulain, and other immunosuppressive agents that act by suppressing the function of responding T cells.

In a further embodiment, the compositions of the invention are administered in combination with an antibiotic agent. Antibiotic agents that may be administered with the compositions of the invention include, but are not limited to, tetracycline, metronidazole, amoxicillin, beta-lactamases, aminoglycosides, macrolides, quinolones, fluoroquinolones, cephalosporins, erythromycin, ciprofloxacin, and streptomycin.

In an additional embodiment, the compositions of the invention are administered alone or in combination with an anti-inflammatory agent. Anti-inflammatory agents that may be administered with the compositions of the invention include, but are not limited to, glucocorticoids and the nonsteroidal anti-inflammatories, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, eacetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

In another embodiment, compositions of the invention are administered in combination with a chemotherapeutic agent. Chemotherapeutic agents that may be administered with the compositions of the invention include, but are not limited to, antibiotic derivatives (e.g., doxorubicin, bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethenyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, and etoposide).

In an additional embodiment, the compositions of the invention are administered in combination with cytokines. Cytokines that may be administered with the compositions of the invention include, but are not limited to, IL2, IL3, IL4, IL5, IL6, IL7, IL10, IL12, IL13, IL15, anti-CD40, CD40L, IFN-gamma and TNF-alpha.

In an additional embodiment, the compositions of the invention are administered in combination with angiogenic proteins. Angiogenic proteins that may be administered with the compositions of the invention include, but are not limited to, Glioma Derived Growth Factor (GDGF), as disclosed in European Patent Number EP-399816; Platelet Derived Growth Factor-A (PDGF-A), as disclosed in European Patent Number EP-682110; Platelet Derived Growth Factor-B (PDGF-B), as disclosed in European Patent Number EP-282317; Placental Growth Factor (PlGF), as disclosed in International Publication Number WO 92/06194; Placental Growth Factor-2 (PlGF-2), as disclosed in Hauser et al., Growth Factors, 4:259-268 (1993); Vascular Endothelial Growth Factor (VEGF), as disclosed in International Publication Number WO 90/13649; Vascular Endothelial Growth Factor-A (VEGF-A), as disclosed in European Patent Number EP-506477; Vascular Endothelial Growth Factor-2 (VEGF-2), as disclosed in International Publication Number WO 96/39515; Vascular Endothelial Growth Factor B-186 (VEGF-B 186), as disclosed in International Publication Number WO 96/26736; Vascular Endothelial Growth Factor-D (VEGF-D), as disclosed in International Publication Number WO 98/02543; Vascular Endothelial Growth Factor-D (VEGF-D), as disclosed in International Publication Number WO 98/07832; and Vascular Endothelial Growth Factor-E (VEGF-E), as disclosed in German Patent Number DE19639601. The above mentioned references are incorporated herein by reference herein.

In an additional embodiment, the compositions of the invention are administered in combination with Fibroblast Growth Factors. Fibroblast Growth Factors that may be administered with the compositions of the invention include, but are not limited to, FGF-1, FGF-2, FGF-3, FGF4, FGF-5, FGF-6, FGF-7, FGF-8, FGF-9, FGF-10, FGF-11, FGF-12, FGF-13, FGF-14, and FGF-15.

In additional embodiments, the compositions of the invention are administered in combination with other therapeutic or prophylactic regimens, such as, for example, radiation therapy.

### Example 27

### Method of Treating Decreased Levels of METH1 or METH2

The present invention relates to a method for treating an individual in need of a decreased level of METH1 or METH2 activity in the body comprising, administering to such an individual a composition comprising a therapeutically effective amount of METH1 or METH2 antagonist. Preferred antagonists for use in the present invention are METH1 or METH2-specific antibodies.

Moreover, it will be appreciated that conditions caused by a decrease in the standard or normal expression level of METH1 or METH2 in an individual can be treated by administering METH1 or METH2, preferably in the secreted form. Thus, the invention also provides a method of treatment of an individual in need of an increased level of METH1 or METH2 polypeptide comprising administering to such an individual a pharmaceutical composition comprising an amount of METH1 or METH2 to increase the activity level of METH1 or METH2 in such an individual.

For example, a patient with decreased levels of METH1 or METH2 polypeptide receives a daily dose 0.1-100 ug/kg of the polypeptide for six consecutive days. Preferably, the polypeptide is in the secreted form. The exact details of the dosing scheme, based on administration and formulation, are provided in Example 26.

### Example 28

### Method of Treating Increased Levels of METH1 or METH2

The present invention also relates to a method for treating an individual in need of an increased level of METH1 or METH2 activity in the body comprising administering to such an individual a composition comprising a therapeutically effective amount of METH1 or METH2 or an agonist thereof.

Antisense technology is used to inhibit production of METH1 or METH2. This technology is one example of a method of decreasing levels of METH1 or METH2 polypeptide, preferably a secreted form, due to a variety of etiologies, such as cancer.

For example, a patient diagnosed with abnormally increased levels of METH1 or METH2 is administered intravenously antisense polynucleotides at 0.5, 1.0, 1.5, 2.0 and 3.0 mg/kg day for 21 days. This treatment is repeated after a 7-day rest period if the treatment was well tolerated. The formulation of the antisense polynucleotide is provided in Example 26.

### Example 29

### Method of Treatment Using Gene Therapy—ex vivo

One method of gene therapy transplants fibroblasts, which are capable of expressing METH1 or METH2 polypeptides, onto a patient. Generally, fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin) is added. The flasks are then incubated at 37° C. for approximately one week.

At this time, fresh media is added and subsequently changed every several days. After an additional two weeks in culture, a monolayer of fibroblasts emerge. The monolayer is trypsinized and scaled into larger flasks. pMV-7 (Kirschmeier, P. T. et al., DNA 7:219-25 (1988)), flanked by the long terminal repeats of the Moloney murine sarcoma virus, is digested with EcoRI and HindIII and subsequently treated with calf intestinal phosphatase. The linear vector is fractionated on agarose gel and purified, using glass beads.

The cDNA encoding METH1 or METH2 can be amplified using PCR primers which correspond to the 5′ and 3′ end sequences respectively as set forth in Example 5. Preferably, the 5′ primer contains an EcoRI site and the 3′ primer includes a HindIII site. Equal quantities of the Moloney murine sarcoma virus linear backbone and the amplified EcoRI and HindIII fragment are added together, in the presence of T4 DNA ligase. The resulting mixture is maintained under conditions appropriate for ligation of the two fragments. The ligation mixture is then used to transform bacteria HB 101, which are then plated onto agar containing kanamycin for the purpose of confirming that the vector contains properly inserted METH1 or METH2.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the METH1 or METH2 gene is then added to the media and the packaging cells transduced with the vector. The packaging cells now produce infectious viral particles containing the METH1 or METH2 gene (the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his. Once the fibroblasts have been efficiently infected, the fibroblasts are analyzed to determine whether METH1 or METH2 protein is produced.

The engineered fibroblasts are then transplanted onto the host, either alone or after having been grown to confluence on cytodex 3 microcarrier beads.

### Example 30

### Method of Treatment Using Gene Therapy—in vivo

Another aspect of the present invention is using in vivo gene therapy methods to treat disorders, diseases and conditions. The gene therapy method relates to the introduction of naked nucleic acid (DNA, RNA, and antisense DNA or RNA) METH1 or METH2 sequences into an animal to increase or decrease the expression of the METH1 or METH2 polypeptide. The METH1 or METH2 polynucleotide may be operatively linked to a promoter or any other genetic elements necessary for the expression of the METH1 or METH2 polypeptide by the target tissue. Such gene therapy and delivery techniques and methods are known in the art, see, for example, WO 90/11092, WO98/11779; U.S. Pat. No. 5,693,622, 5,705,151, 5,580,859; Tabata, H. et al. (1997) Cardiovasc. Res. 35(3):470-479, Chao, J. et al. (1997) Pharmacol. Res. 35(6):517-522, Wolff, J. A. (1997) Neuromuscul. Disord. 7(5):314-318, Schwartz, B. et al. (1996) Gene Ther. 3(5):405-411, Tsurumi, Y. et al. (1996) Circulation 94(12):3281-3290 (incorporated herein by reference).

The METH1 or METH2 polynucleotide constructs may be delivered by any method that delivers injectable materials to the cells of an animal, such as, injection into the interstitial space of tissues (heart, muscle, skin, lung, liver, intestine and the like). The METH1 or METH2 polynucleotide constructs can be delivered in a pharmaceutically acceptable liquid or aqueous carrier.

The term “naked” polynucleotide, DNA or RNA, refers to sequences that are free from any delivery vehicle that acts to assist, promote, or facilitate entry into the cell, including viral sequences, viral particles, liposome formulations, lipofectin or precipitating agents and the like. However, the METH1 or METH2 polynucleotides may also be delivered in liposome formulations (such as those taught in Felgner, P. L. et al. (1995) Ann. NY Acad. Sci. 772:126-139 and Abdallah, B. et al. (1995) Biol. Cell 85(1):1-7) which can be prepared by methods well known to those skilled in the art.

The METH1 or METH2 polynucleotide vector constructs used in the gene therapy method are preferably constructs that will not integrate into the host genome nor will they contain sequences that allow for replication. Any strong promoter known to those skilled in the art can be used for driving the expression of DNA. Unlike other gene therapies techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The METH1 or METH2 polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked METH1 or METH2 polynucleotide injection, an effective dosage amount of DNA or RNA will be in the range of from about 0.0005 g/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration. The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked METH1 or METH2polynucleotide constructs can be delivered to arteries during angioplasty by the catheter used in the procedure.

The dose response effects of injected METH1 or METH2 polynucleotide in muscle in vivo is determined as follows. Suitable METH1 or METH2 template DNA for production of mRNA coding for METH1 or METH2 polypeptide is prepared in accordance with a standard recombinant DNA methodology. The template DNA, which may be either circular or linear, is either used as naked DNA or complexed with liposomes. The quadriceps muscles of mice are then injected with various amounts of the template DNA.

Five to six week old female and male Balb/C mice are anesthetized by intraperitoneal injection with 0.3 ml of 2.5% Avertin. A 1.5 cm incision is made on the anterior thigh, and the quadriceps muscle is directly visualized. The METH1 or METH2 template DNA is injected in 0.1 ml of carrier in a 1 cc syringe through a 27 gauge needle over one minute, approximately 0.5 cm from the distal insertion site of the muscle into the knee and about 0.2 cm deep. A suture is placed over the injection site for future localization, and the skin is closed with stainless steel clips.

After an appropriate incubation time (e.g., 7 days) muscle extracts are prepared by excising the entire quadriceps. Every fifth 15 um cross-section of the individual quadriceps muscles is histochemically stained for METH1 or METH2 protein expression. A time course for METH1 or METH2 protein expression may be done in a similar fashion except that quadriceps from different mice are harvested at different times. Persistence of METH1 or METH2 DNA in muscle following injection may be determined by Southern blot analysis after preparing total cellular DNA and HIRT supernatants from injected and control mice. The results of the above experimentation in mice can be use to extrapolate proper dosages and other treatment parameters in humans and other animals using METH1 or METH2 naked DNA.

### Example 31

### Gene Therapy Using Endogenous METH1 and/or METH2 Gene

Another method of gene therapy according to the present invention involves operably associating the endogenous METH1 and/or METH2 sequence with a promoter via homologous recombination as described, for example, in U.S. Pat. No. 5,641,670, issued Jun. 24, 1997; International Publication No. WO 96/29411, published Sep. 26, 1996; International Publication No. WO 94/12650, published Aug. 4, 1994; Koller et al., Proc. Natl. Acad. Sci. USA 86:8932-8935 (1989); and Zijlstra et al., Nature 342:435-438 (1989). This method involves the activation of a gene which is present in the target cells, but which is not expressed in the cells, or is expressed at a lower level than desired.

Polynucleotide constructs are made which contain a promoter and targeting sequences, which are homologous to the 5′ non-coding sequence of endogenous METH1 and/or METH12, flanking the promoter. The targeting sequence will be sufficiently near the 5′ end of METH1 and/or METH2 so the promoter will be operably linked to the endogenous sequence upon homologous recombination. The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter.

The amplified promoter and the amplified targeting sequences are digested with the appropriate restriction enzymes and subsequently treated with calf intestinal phosphatase. The digested promoter and digested targeting sequences are added together in the presence of T4 DNA ligase. The resulting mixture is maintained under conditions appropriate for ligation of the two fragments. The construct is size fractionated on an agarose gel then purified by phenol extraction and ethanol precipitation.

In this Example, the polynucleotide constructs are administered as naked polynucleotides via electroporation. However, the polynucleotide constructs may also be administered with transfection-facilitating agents, such as liposomes, viral sequences, viral particles, precipitating agents, etc. Such methods of delivery are known in the art.

Once the cells are transfected, homologous recombination will take place which results in the promoter being operably linked to the endogenous METH1 and/or METH2 sequence. This results in the expression of METH1 and/or METH2 in the cell. Expression may be detected by immunological staining, or any other method known in the art. Fibroblasts are obtained from a subject by skin biopsy. The resulting tissue is placed in DMEM+10% fetal calf serum. Exponentially growing or early stationary phase fibroblasts are trypsinized and rinsed from the plastic surface with nutrient medium. An aliquot of the cell suspension is removed for counting, and the remaining cells are subjected to centrifugation. The supernatant is aspirated and the pellet is resuspended in 5 ml of electroporation buffer (20 mM HEPES pH 7.3, 137 mM NaCl, 5 mM KCl, 0.7 mM Na₂ HPO4, 6 mM dextrose). The cells are recentrifuged, the supernatant aspirated, and the cells resuspended in electroporation buffer containing 1 mg/ml acetylated bovine serum albumin. The final cell suspension contains approximately 3×10⁶ cells/ml. Electroporation should be performed immediately following resuspension.

Plasmid DNA is prepared according to standard techniques. For example, to construct a plasmid for targeting to the METH1 and/or METH2 locus, plasmid pUC18 (MBI Fermentas, Amherst, N.Y.) is digested with HindIII. The CMV promoter is amplified by PCR with an XbaI site on the 5′ end and a BamHI site on the 3′end. Two METH1 and/or METH2 non-coding sequences are amplified via PCR: one METH1 and/or METH2 non-coding sequence (METH1 and/or METH2 fragment 1) is amplified with a HindIII site at the 5′ end and an Xba site at the 3′end; the other METH1 and/or METH2 non-coding sequence (METH1 and/or METH2 fragment 2) is amplified with a BamHI site at the 5′end and a HindIII site at the 3′end. The CMV promoter and METH1 and/or METH2 fragments are digested with the appropriate enzymes (CMV promoter—XbaI and BamHI; METH1 and/or METH2 fragment 1—XbaI; METH1 and/or METH2 fragment 2-BamI) and ligated together. The resulting ligation product is digested with HindIII, and ligated with the HindIII-digested pUC18 plasmid.

Plasmid DNA is added to a sterile cuvette with a 0.4 cm electrode gap (Bio-Rad). The final DNA concentration is generally at least 120 μml. 0.5 ml of the cell suspension (containing approximately 1.5×10⁶ cells) is then added to the cuvette, and the cell suspension and DNA solutions are gently mixed. Electroporation is performed with a Gene-Pulser apparatus (Bio-Rad). Capacitance and voltage are set at 960 μF and 250-300 V, respectively. As voltage increases, cell survival decreases, but the percentage of surviving cells that stably incorporate the introduced DNA into their genome increases dramatically. Given these parameters, a pulse time of approximately 14-20 mSec should be observed.

Electroporated cells are maintained at room temperature for approximately 5 min, and the contents of the cuvette are then gently removed with a sterile transfer pipette. The cells are added directly to 10 ml of prewarmed nutrient media (DMEM with 15% calf serum) in a 10 cm dish and incubated at 37° C. The following day, the media is aspirated and replaced with 10 ml of fresh media and incubated for a further 16-24 hours.

The engineered fibroblasts are then injected into the host, either alone or after having been grown to confluence on cytodex 3 microcarrier beads. The fibroblasts now produce the protein product. The fibroblasts can then be introduced into a patient as described above.

### Example 32

### METH1 and/or METH2 Transgenic Animals

The METH1 and/or METH2 polypeptides can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce the founder lines of transgenic animals. Such techniques include, but are not limited to, pronuclear microinjection (Paterson et al., Appl. Microbiol. Biotechnol. 40:691-698 (1994); Carver et al., Biotechnology (NY) 11:1263-1270 (1993); Wright et al., Biotechnology (NY) 9:830-834 (1991); and Hoppe et al., U.S. Pat. No. 4,873,191 (1989)); retrovirus mediated gene transfer into germ lines (Van der Putten et al., Proc. Natl. Acad. Sci. USA 82:6148-6152 (1985)), blastocysts or embryos; gene targeting in embryonic stem cells (Thompson et al., Cell 56:313-321 (1989)); electroporation of cells or embryos (Lo, 1983, Mol Cell. Biol. 3:1803-1814 (1983)); introduction of the polynucleotides of the invention using a gene gun (see, e.g., Ulmer et al., Science 259:1745 (1993); introducing nucleic acid constructs into embryonic pleuripotent stem cells and transferring the stem cells back into the blastocyst; and sperm-mediated gene transfer (Lavitrano et al., Cell 57:717-723 (1989); etc. For a review of such techniques, see Gordon, “Transgenic Animals,” Intl. Rev. Cytol. 115:171-229 (1989), which is incorporated by reference herein in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al., Nature 380:64-66 (1996); Wilmut et al., Nature 385:810-813 (1997)).

The present invention provides for transgenic animals that carry the transgene in all their cells, as well as animals which carry the transgene in some, but not all their cells, i.e., mosaic animals or chimeric. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred.

Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265:103-106 (1994)). The regulatory sequences required for such a cell-type specific inactivation will depend upon the particular cell type of interest, and will be apparent to those of skill in the art. The contents of each of the documents recited in this paragraph is herein incorporated by reference in its entirety.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR (rt-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of METH1 and/or METH2 polypeptides, studying conditions and/or disorders associated with aberrant METH1 and/or METH2 expression, and in screening for compounds effective in ameliorating such conditions and/or disorders.

### Example 33

### METH1 and/or METH2 Knock-Out Animals

Endogenous METH1 and/or METH2 gene expression can also be reduced by inactivating or “knocking out” the METH1 and/or METH2 gene and/or its promoter using targeted homologous recombination. (E.g., see Smithies et al., Nature 317:230-234 (1985); Thomas & Capecchi, Cell 51:503-512 (1987); Thompson et al., Cell 5:313-321 (1989); each of which is incorporated by reference herein in its entirety). For example, a mutant, non-functional polynucleotide of the invention (or a completely unrelated DNA sequence) flanked by DNA homologous to the endogenous polynucleotide sequence (either the coding regions or regulatory regions of the gene) can be used, with or without a selectable marker and/or a negative selectable marker, to transfect cells that express polypeptides of the invention in vivo. In another embodiment, techniques known in the art are used to generate knockouts in cells that contain, but do not express the gene of interest. Insertion of the DNA construct, via targeted homologous recombination, results in inactivation of the targeted gene. Such approaches are particularly suited in research and agricultural fields where modifications to embryonic stem cells can be used to generate animal offspring with an inactive targeted gene (e.g., see Thomas & Capecchi 1987 and Thompson 1989, supra). However this approach can be routinely adapted for use in humans provided the recombinant DNA constructs are directly administered or targeted to the required site in vivo using appropriate viral vectors that will be apparent to those of skill in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and preferably secretion, of the et al. METH1 and/or METH2 polypeptides. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft;

genetically engineered endothelial cells can be implanted as part of a lymphatic or vascular graft. (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Knock-out animals of the invention have uses which include, but are not limited to, animal model systems useful in elaborating the biological function of METH1 and/or METH2 polypeptides, studying conditions and/or disorders associated with aberrant METH1 and/or METH2 expression, and in screening for compounds effective in ameliorating such conditions and/or disorders.

### Example 34

### Assays Detecting Stimulation or Inhibition of B cell Proliferation and Differentiation

Generation of functional humoral immune responses requires both soluble and cognate signaling between B-lineage cells and their microenvironment. Signals may impart a positive stimulus that allows a B-lineage cell to continue its programmed development, or a negative stimulus that instructs the cell to arrest its current developmental pathway. To date, numerous stimulatory and inhibitory signals have been found to influence B cell responsiveness including IL-2, IL-4, IL-5, IL-6, IL-7, IL-10, IL-13, IL-14 and IL-15. Interestingly, these signals are by themselves weak effectors but can, in combination with various co-stimulatory proteins, induce activation, proliferation, differentiation, homing, tolerance and death among B cell populations.

One of the best studied classes of B-cell co-stimulatory proteins is the TNF-superfamily. Within this family CD40, CD27, and CD30 along with their respective ligands CD154, CD70, and CD153 have been found to regulate a variety of immune responses. Assays which allow for the detection and/or observation of the proliferation and differentiation of these B-cell populations and their precursors are valuable tools in determining the effects various proteins may have on these B-cell populations in terms of proliferation and differentiation. Listed below are two assays designed to allow for the detection of the differentiation, proliferation, or inhibition of B-cell populations and their precursors.

In vitro Assay—Purified METH1 and/or METH2 protein, or truncated forms thereof, is assessed for its ability to induce activation, proliferation, differentiation or inhibition and/or death in B-cell populations and their precursors. The activity of METH1 and/or METH2 protein on purified human tonsillar B cells, measured qualitatively over the dose range from 0.1 to 10,000 ng/mL, is assessed in a standard B-lymphocyte co-stimulation assay in which purified tonsillar B cells are cultured in the presence of either formalin-fixed Staphylococcus aureus Cowan I (SAC) or immobilized anti-human IgM antibody as the priming agent. Second signals such as IL-2 and IL-15 synergize with SAC and IgM crosslinking to elicit B cell proliferation as measured by tritiated-thymidine incorporation. Novel synergizing agents can be readily identified using this assay. The assay involves isolating human tonsillar B cells by magnetic bead (MACS) depletion of CD3-positive cells. The resulting cell population is greater than 95% B cells as assessed by expression of CD45R(B220).

Various dilutions of each sample are placed into individual wells of a 96-well plate to which are added 10⁵ B-cells suspended in culture medium (RPMI 1640 containing 10% FBS, 5×10⁻⁵M 2ME, 10 μg/ml penicillin, 10 μg/ml streptomycin, and 10⁻⁵ dilution of SAC) in a total volume of 150 μt. Proliferation or inhibition is quantitated by a 20 h pulse (1 μCi/well) with ³H-thymidine (6.7 Ci/mM) beginning 72 hours post factor addition. The positive and negative controls are IL2 and medium respectively.

In vivo Assay-BALB/c mice are injected (i.p.) twice per day with buffer only, or 2 mg/Kg of METH1 and/or METH2 protein, or truncated forms thereof. Mice receive this treatment for 4 consecutive days, at which time they are sacrificed and various tissues and serum collected for analyses. Comparison of H&E sections from normal and METH1 and/or METH2 protein-treated spleens identify the results of the activity of METH1 and/or METH2 protein on spleen cells, such as the diffusion of peri-arterial lymphatic sheaths, and/or significant increases in the nucleated cellularity of the red pulp regions, which may indicate the activation of the differentiation and proliferation of B-cell populations. immunohistochemical studies using a B cell marker, anti-CD45R(B220), are used to determine whether any physiological changes to splenic cells, such as splenic disorganization, are due to increased B-cell representation within loosely defined B-cell zones that infiltrate established T-cell regions.

Flow cytometric analyses of the spleens from METH1 and/or METH2 protein-treated mice is used to indicate whether METH1 and/or METH2 protein specifically increases the proportion of ThB+, CD45R(B220)dull B cells over that which is observed in control mice.

Likewise, a predicted consequence of increased mature B-cell representation in vivo is a relative increase in serum Ig titers. Accordingly, serum IgM and IgA levels are compared between buffer and METH1 and/or METH2 protein-treated mice.

The studies described in this example test activity in METH1 and/or METH2 protein. However, one skilled in the art could easily modify the exemplified studies to test the activity of METH1 and/or METH2 polynucleotides (e.g., gene therapy), agonists, and/or antagonists of METH1 and/or METH2.

### Example 35

### T Cell Proliferation Assay

A CD3-induced proliferation assay is performed on PBMCs and is measured by the uptake of ³H-thymidine. The assay is performed as follows. Ninety-six well plates are coated with 100 μl/well of mAb to CD3 (HIT3a, Pharmingen) or isotype-matched control mAb (1333.1) overnight at 4° C. (1 mg/ml in 0.05M bicarbonate buffer, pH 9.5), then washed three times with PBS. PBMC are isolated by F/H gradient centrifugation from human peripheral blood and added to quadruplicate wells (5×10⁴/well) of mAb coated plates in RPMI containing 10% FCS and P/S in the presence of varying concentrations of et al. METH1 and/or METH2 protein (total volume 200 μl). Relevant protein buffer and medium alone are controls. After 48 hr. culture at 37° C., plates are spun for 2 min. at 1000 rpm and 100 μl of supernatant is removed and stored -20° C. for measurement of IL-2 (or other cytokines) if effect on proliferation is observed. Wells are supplemented with 100 μl of medium containing 0.5 μCi of ³H-thymidine and cultured at 37° C. for 18-24 hr. Wells are harvested and incorporation of ³H-thymidine used as a measure of proliferation. Anti-CD3 alone is the positive control for proliferation. IL-2 (100 U/ml) is also used as a control which enhances proliferation. Control antibody which does not induce proliferation of T cells is used as the negative control for the effects of METH1 and/or METH2 proteins.

The studies described in this example tested activity in METH1 and/or METH2 protein. However, one skilled in the art could easily modify the exemplified studies to test the activity of METH1 and/or METH2 polynucleotides (e.g., gene therapy), agonists, and/or antagonists of METH1 and/or METH2.

### Example 36

### Effect of METH1 and/or METH2 on the Expression of MHC Class II Costimulatory and Adhesion Molecules and Cell Differentiation of Monocytes and Monocyte-Derived Human Dendritic Cells

Dendritic cells are generated by the expansion of proliferating precursors found in the peripheral blood: adherent PBMC or elutriated monocytic fractions are cultured for 7-10 days with GM-CSF (50 ng/ml) and IL-4 (20 ng/ml). These dendritic cells have the characteristic phenotype of immature cells (expression of CD1, CD80, CD86, CD40 and -MHC class II antigens). Treatment with activating factors, such as TNF-α, causes a rapid change in surface phenotype (increased expression of MHC class I and II, costimulatory and adhesion molecules, downregulation of FCγ R11, upregulation of CD83). These changes correlate with increased antigen-presenting capacity and with functional maturation of the dendritic cells.

FACS analysis of surface antigens is performed as follows. Cells are treated 1-3 days with increasing concentrations of METH1 and/or METH2 or LPS (positive control), washed with PBS containing 1% BSA and 0.02 mM sodium azide, and then incubated with 1:20 dilution of appropriate FITC- or PE-labeled monoclonal antibodies for 30 minutes at 4° C. After an additional wash, the labeled cells are analyzed by flow cytometry on a FACScan (Becton Dickinson).

Effect on the production of cytokines. Cytokines generated by dendritic cells, in particular IL-12, are important in the initiation of T-cell dependent immune responses. IL-12 strongly influences the development of Th1 helper T-cell immune response, and induces cytotoxic T and NK cell function. An ELISA is used to measure the IL-12 release as follows. Dendritic cells (106/ml) are treated with increasing concentrations of METH1 and/or METH2 for 24 hours. LPS (100 ng/ml) is added to the cell culture as positive control. Supernatants from the cell cultures are then collected and analyzed for IL-12 content using commercial ELISA kit (e.g, R & D Systems (Minneapolis, Minn.)). The standard protocols provided with the kits are used.

Effect on the expression of MHC Class II, costimulatory and adhesion molecules. Three major families of cell surface antigens can be identified on monocytes: adhesion molecules, molecules involved in antigen presentation, and Fc receptor. Modulation of the expression of MHC class II antigens and other costimulatory molecules, such as B7 and ICAM-1, may result in changes in the antigen presenting capacity of monocytes and ability to induce T cell activation. Increase expression of Fc receptors may correlate with improved monocyte cytotoxic activity, cytokine release and phagocytosis.

FACS analysis is used to examine the surface antigens as follows. Monocytes are treated 1-5 days with increasing concentrations of METH1 and/or METH2 or LPS (positive control), washed with PBS containing 1% BSA and 0.02 mM sodium azide, and then incubated with 1:20 dilution of appropriate FITC- or PE-labeled monoclonal antibodies for 30 minutes at 4° C. After an additional wash, the labeled cells are analyzed by flow cytometry on a FACScan (Becton Dickinson).

Monocyte activation and/or increased survival. Assays for molecules that activate (or alternatively, inactivate) monocytes and/or increase monocyte survival (or alternatively, decrease monocyte survival) are known in the art and may routinely be applied to determine whether a molecule of the invention functions as an inhibitor or activator of monocytes. METH1 and/or METH2, agonists, or antagonists of METH1 and/or METH2 can be screened using the three assays described below. For each of these assays, Peripheral blood mononuclear cells (PBMC) are purified from single donor leukopacks (American Red Cross, Baltimore, Md.) by centrifugation through a Histopaque gradient (Sigma). Monocytes are isolated from PBMC by counterflow centrifugal elutriation.

1. Monocyte Survival Assay. Human peripheral blood monocytes progressively lose viability when cultured in absence of serum or other stimuli. Their death results from internally regulated process (apoptosis). Addition to the culture of activating factors, such as TNF-alpha dramatically improves cell survival and prevents DNA fragmentation.

Propidium iodide (PI) staining is used to measure apoptosis as follows. Monocytes are cultured for 48 hours in polypropylene tubes in serum-free medium (positive control), in the presence of 100 ng/ml TNF-alpha (negative control), and in the presence of varying concentrations of the compound to be tested. Cells are suspended at a concentration of 2×10⁶/ml in PBS containing PI at a final concentration of 5 μg/ml, and then incubated at room temperature for 5 minutes before FACScan analysis. PI uptake has been demonstrated to correlate with DNA fragmentation in this experimental paradigm.

2. Effect on cytokine release. An important function of monocytes/macrophages is their regulatory activity on other cellular populations of the immune system through the release of cytokines after stimulation. An ELISA to measure cytokine release is performed as follows. Human monocytes are incubated at a density of 5×10⁵ cells/ml with increasing concentrations of METH1 and/or METH2 and under the same conditions, but in the absence of METH1 and/or METH2. For IL-12 production, the cells are primed overnight with IFN (100 U/ml) in presence of METH1 and/or METH2. LPS (10 ng/ml) is then added. Conditioned media are collected after 24 h and kept frozen until use. Measurement of TNF-alpha, IL-10, MCP-1 and IL-8 is then performed using a commercially available ELISA kit (e.g, R & D Systems (Minneapolis, Minn.)) and applying the standard protocols provided with the kit. 3. Oxidative burst. Purified monocytes are plated in 96-w plate at 2-1×10⁵ cell/well. Increasing concentrations of METH1 and/or METH12 are added to the wells in a total volume of 0.2 ml culture medium (RPMI 1640+10% FCS, glutamine and antibiotics). After 3 days incubation, the plates are centrifuged and the medium is removed from the wells. To the macrophage monolayers, 0.2 mil per well of phenol red solution (140 mM NaCl, 10 mM potassium phosphate buffer pH 7.0, 5.5 mM dextrose, 0.56 mM phenol red and 19 U/ml of HRPO) is added, together with the stimulant (200 nM PMA). The plates are incubated at 37° C. for 2 hours and the reaction is stopped by adding 20 μl N NaOH per well. The absorbance is read at 610 nm. To calculate the amount of H₂O₂ produced by the macrophages, a standard curve of a H₂O₂ solution of known molarity is performed for each experiment.

The studies described in this example tested activity in METH1 and/or METH2 protein. However, one skilled in the art could easily modify the exemplified studies to test the activity of METH1 and/or METH2 polynucleotides (e.g., gene therapy), agonists, and/or antagonists of METH1 and/or METH2.

### Example 37

### METH1 and/or METH2 Biological Effects

Astrocyte and Neuronal Assays. Recombinant METH1 and/or METH2, expressed in Escherichia coli and purified as described above, can be tested for activity in promoting the survival, neurite outgrowth, or phenotypic differentiation of cortical neuronal cells and for inducing the proliferation of glial fibrillary acidic protein immunopositive cells, astrocytes. The selection of cortical cells for the bioassay is based on the prevalent expression of FGF-1 and FGF-2 in cortical structures and on the previously reported enhancement of cortical neuronal survival resulting from FGF-2 treatment. A thymidine incorporation assay, for example, can be used to elucidate METH1 and/or METH2's activity on these cells.

Moreover, previous reports describing the biological effects of FGF-2 (basic FGF) on cortical or hippocampal neurons in vitro have demonstrated increases in both neuron survival and neurite outgrowth (Walicke, P. et al., “Fibroblast growth factor promotes survival of dissociated hippocampal neurons and enhances neurite extension.” Proc. Natl. Acad. Sci. USA 83:3012-3016. (1986), assay herein incorporated by reference in its entirety). However, reports from experiments done on PC-12 cells suggest that these two responses are not necessarily synonymous and may depend on not only which FGF is being tested but also on which receptor(s) are expressed on the target cells. Using the primary cortical neuronal culture paradigm, the ability of METH1 and/or METH2 to induce neurite outgrowth can be compared to the response achieved with FGF-2 using, for example, a thymidine incorporation assay.

Fibroblast and endothelial cell assays. Human lung fibroblasts are obtained from Clonetics (San Diego, Calif.) and maintained in growth media from Clonetics. Dermal microvascular endothelial cells are obtained from Cell Applications (San Diego, Calif.). For proliferation assays, the human lung fibroblasts and dermal microvascular endothelial cells can be cultured at 5,000 cells/well in a 96-well plate for one day in growth medium. The cells are then incubated for one day in 0.1% BSA basal medium. After replacing the medium with fresh 0.1% BSA medium, the cells are incubated with the test proteins for 3 days. Alamar Blue (Alamar Biosciences, Sacramento, Calif.) is added to each well to a final concentration of 10%. The cells are incubated for 4 hr. Cell viability is measured by reading in a CytoFluor fluorescence reader. For the PGE₂ assays, the human lung fibroblasts are cultured at 5,000 cells/well in a 96-well plate for one day. After a medium change to 0.1% BSA basal medium, the cells are incubated with FGF-2 or METH1 and/or METH2 with or without IL-1a for 24 hours. The supernatants are collected and assayed for PGE₂ by EIA kit (Cayman, Ann Arbor, Mich.). For the IL-6 assays, the human lung fibroblasts are cultured at 5,000 cells/well in a 96-well plate for one day. After a medium change to 0.1% BSA basal medium, the cells are incubated with FGF-2 or METH1 and/or METH2 with or without IL-1α for 24 hours. The supernatants are collected and assayed for IL-6 by ELISA kit (Endogen, Cambridge, Mass.).

Human lung fibroblasts are cultured with FGF-2 or METH1 and/or METH2 for 3 days in basal medium before the addition of Alamar Blue to assess effects on growth of the fibroblasts. FGF-2 should show a stimulation at 10-2500 ng/ml which can be used to compare stimulation with METH1 and/or METH2.

Parkinson Models. The loss of motor function in Parkinson's disease is attributed to a deficiency of striatal dopamine resulting from the degeneration of the nigrostriatal dopaminergic projection neurons. An animal model for Parkinson's that has been extensively characterized involves the systemic administration of 1-methyl-4 phenyl 1,2,3,6-tetrahydropyridine (MPTP). In the CNS, MPTP is taken-up by astrocytes and catabolized by monoamine oxidase B to 1-methyl-4-phenyl pyridine (MPP⁺) and released. Subsequently, MPP⁺ is actively accumulated in dopaminergic neurons by the high-affinity reuptake transporter for dopamine. MPP⁺ is then concentrated in mitochondria by the electrochemical gradient and selectively inhibits nicotidamide adenine disphosphate: ubiquinone oxidoreductionase (complex I), thereby interfering with electron transport and eventually generating oxygen radicals.

It has been demonstrated in tissue culture paradigms that FGF-2 (basic FGF) has trophic activity towards nigral dopaminergic neurons (Ferrari et al., Dev. Biol. 1989). Recently, Dr. Unsicker's group has demonstrated that administering FGF-2 in gel foam implants in the striatum results in the near complete protection of nigral dopaminergic neurons from the toxicity associated with MPTP exposure (Otto and Unsicker, J. Neuroscience, 1990).

Based on the data with FGF-2, METH1 and/or METH2 can be evaluated to determine whether it has an action similar to that of FGF-2 in enhancing dopaminergic neuronal survival in vitro and it can also be tested in vivo for protection of dopaminergic neurons in the striatum from the damage associated with MPTP treatment. The potential effect of METH1 and/or METH2 is first examined in vitro in a dopaminergic neuronal cell culture paradigm. The cultures are prepared by dissecting the midbrain floor plate from gestation day 14 Wistar rat embryos. The tissue is dissociated with trypsin and seeded at a density of 200,000 cells/cm² on polyorthinine-laminin coated glass coverslips. The cells are maintained in Dulbecco's Modified Eagle's medium and F12 medium containing hormonal supplements (N1). The cultures are fixed with paraformaldehyde after 8 days in vitro and are processed for tyrosine hydroxylase, a specific marker for dopminergic neurons, immunohistochemical staining. Dissociated cell cultures are prepared from embryonic rats. The culture medium is changed every third day and the factors are also added at that time. Since the dopaminergic neurons are isolated from animals at gestation day 14, a developmental time which is past the stage when the dopaminergic precursor cells are proliferating, an increase in the number of tyrosine hydroxylase immunopositive neurons would represent an increase in the number of dopaminergic neurons surviving in vitro. Therefore, if METH1 and/or METH2 acts to prolong the survival of dopaminergic neurons, it would suggest that METH1 and/or METH2 may be involved in Parkinson's Disease.

The studies described in this example tested activity in METH1 and/or METH2 protein. However, one skilled in the art could easily modify the exemplified studies to test the activity of METH1 and/or METH2 polynucleotides (e.g., gene therapy), agonists, and/or antagonists of METH1 and/or METH2.

### Example 38

### The Effect of METH1 and/or METH2 on the Growth of Vascular Endothelial Cells

On day 1, human umbilical vein endothelial cells (HUVEC) are seeded at 2-5×10⁴ cells/35 mm dish density in M199 medium containing 4% fetal bovine serum (FBS), 16 units/ml heparin, and 50 units/ml endothelial cell growth supplements (ECGS, Biotechnique, Inc.). On day 2, the medium is replaced with M199 containing 10% FBS, 8 units/ml heparin. METH1 and/or METH2 protein of SEQ ID NO. 2, and positive controls, such as VEGF and basic FGF (bFGF) are added, at varying concentrations. On days 4 and 6, the medium is replaced. On day 8, cell number is determined with a Coulter Counter.

An increase in the number of HUVEC cells indicates that METH1 and/or METH2 may proliferate vascular endothelial cells.

The studies described in this example tested activity in METH1 and/or METH2 protein. However, one skilled in the art could easily modify the exemplified studies to test the activity of METH1 and/or METH2 polynucleotides (e.g., gene therapy), agonists, and/or antagonists of METH1 and/or METH2.

### Example 39

### Stimulatory Effect of METH1 and/or METH2 on the Proliferation of Vascular Endothelial Cells

For evaluation of mitogenic activity of growth factors, the calorimetric MTS (3-(4,5-dimethylthiazol-2-yl)-5-(3-carboxymethoxyphenyl)-2-(4-sulfophenyl)₂H-tetrazolium) assay with the electron coupling reagent PMS (phenazine methosulfate) is performed (CellTiter 96 AQ, Promega). Cells are seeded in a 96-well plate (5,000 cells/well) in 0.1 mL serum-supplemented medium and are allowed to attach overnight. After serum-starvation for 12 hours in 0.5% FBS, conditions (bFGF, VEGF₁₆₅ or METH1 and/or METH2 in 0.5% FBS) with or without Heparin (8 U/ml) are added to wells for 48 hours. 20 mg of MTS/PMS mixture (1:0.05) are added per well and allowed to incubate for 1 hour at 37° C. before measuring the absorbance at 490 nm in an ELISA plate reader. Background absorbance from control wells (some media, no cells) is subtracted, and seven wells are performed in parallel for each condition. See, Leak et al. in vitro Cell. Dev. Biol. 30A:512-518 (1994).

The studies described in this example tested activity in METH1 and/or METH2 protein. However, one skilled in the art could easily modify the exemplified studies to test the activity of METH1 and/or METH2 polynucleotides (e.g., gene therapy), agonists, and/or antagonists of METH1 and/or METH2.

### Example 40

### Inhibition of PDGF-induced Vascular Smooth Muscle Cell Proliferation Stimulatory Effect

HAoSMC proliferation can be measured, for example, by BrdUrd incorporation. Briefly, subconfluent, quiescent cells grown on the 4-chamber slides are transfected with CRP or FITC-labeled AT2-3LP. Then, the cells are pulsed with 10% calf serum and 6 mg/ml BrdUrd. After 24 h, immunocytochemistry is performed by using BrdUrd Staining Kit (Zymed Laboratories). In brief, the cells are incubated with the biotinylated mouse anti-BrdUrd antibody at 4° C. for 2 h after being exposed to denaturing solution and then incubated with the streptavidin-peroxidase and diaminobenzidine. After counterstaining with hematoxylin, the cells are mounted for microscopic examination, and the BrdUrd-positive cells are counted. The BrdUrd index is calculated as a percent of the BrdUrd-positive cells to the total cell number. In addition, the simultaneous detection of the BrdUrd staining (nucleus) and the FITC uptake (cytoplasm) is performed for individual cells by the concomitant use of bright field illumination and dark field-UV fluorescent illumination. See, Hayashida et al., J. Biol. Chem. 6:271(36):21985-21992 (1996).

The studies described in this example tested activity in METH1 and/or METH2 protein. However, one skilled in the art could easily modify the exemplified studies to test the activity of METH1 and/or METH2 polynucleotides (e.g., gene therapy), agonists, and/or antagonists of METH1 and/or METH2.

### Example 41

### Stimutation of Endothelial Migration

This example will be used to explore the possibility that METH1 and/or METH2 may stimulate lymphatic endothelial cell migration.

Endothelial cell migration assays are performed using a 48 well microchemotaxis chamber (Neuroprobe Inc., Cabin John, M D; Falk, W., et al., J. Immunological Methods 1980;33:239-247). Polyvinylpyrrolidone-free polycarbonate filters with a pore size of 8 um (Nucleopore Corp. Cambridge, Mass.) are coated with 0.1% gelatin for at least 6 hours at room temperature and dried under sterile air. Test substances are diluted to appropriate concentrations in M199 supplemented with 0.25% bovine serum albumin (BSA), and 25 μl of the final dilution is placed in the lower chamber of the modified Boyden apparatus. Subconfluent, early passage (2-6) HUVEC or BMEC cultures are washed and trypsinized for the minimum time required to achieve cell detachment. After placing the filter between lower and upper chamber, 2.5×10⁵ cells suspended in 50 μl M199 containing 1% FBS are seeded in the upper compartment. The apparatus is then incubated for 5 hours at 37° C. in a humidified chamber with 5% CO₂ to allow cell migration. After the incubation period, the filter is removed and the upper side of the filter with the non-migrated cells is scraped with a rubber policeman. The filters are fixed with methanol and stained with a Giemsa solution (Diff-Quick, Baxter, McGraw Park, Ill.). Migration is quantified by counting cells of three random high-power fields (40×) in each well, and all groups are performed in quadruplicate.

The studies described in this example tested activity in METH1 and/or METH2 protein. However, one skilled in the art could easily modify the exemplified studies to test the activity of METH1 and/or METH2 polynucleotides (e.g., gene therapy), agonists, and/or antagonists of METH1 and/or METH2.

### Example 42

### Stimulation of Nitric Oxide Production by Endothelial Cells

Nitric oxide released by the vascular endothelium is believed to be a mediator of vascular endothelium relaxation. Thus, METH1 and/or METH2 activity can be assayed by determining nitric oxide production by endothelial cells in response to METH1 and/or METH2.

Nitric oxide is measured in 96-well plates of confluent microvascular endothelial cells after 24 hours starvation and a subsequent 4 hr exposure to various levels of a positive control (such as VEGF-1) and METH1 and/or METH2. Nitric oxide in the medium is determined by use of the Griess reagent to measure total nitrite after reduction of nitric oxide-derived nitrate by nitrate reductase. The effect of METH1 and/or METH2 on nitric oxide release is examined on HUVEC.

Briefly, NO release from cultured HUVEC monolayer is measured with a NO-specific polarographic electrode connected to a NO meter (Iso-NO, World Precision Instruments Inc.) (1049). Calibration of the NO elements is performed according to the following equation:

2KNO₂+2KI+2H₂SO₄62NO+I₂+2H₂O+2K₂SO₄

The standard calibration curve is obtained by adding graded concentrations of KNO₂ (0, 5, 10, 25, 50, 100, 250, and 500 mmol/L) into the calibration solution containing KI and 1H2SO₄. The specificity of the Iso-NO electrode to NO is previously determined by measurement of NO from authentic NO gas (1050). The culture medium is removed and HUVECs are washed twice with Dulbecco's phosphate buffered saline. The cells are then bathed in 5 ml of filtered Krebs-Henseleit solution in 6-well plates, and the cell plates are kept on a slide warmer (Lab Line Instruments Inc.) to maintain the temperature at 37° C. The NO sensor probe is inserted vertically into the wells, keeping the tip of the electrode 2 mm under the surface of the solution, before addition of the different conditions. S-nitroso acetyl penicillamin (SNAP) is used as a positive control. The amount of released NO is expressed as picomoles per 1×10⁶ endothelial cells. All values reported are means of four to six measurements in each group (number of cell culture wells). See, Leak et al. Biochem. and Biophys. Res. Comm. 217:96-105 (1995).

The studies described in this example tested activity in METH1 and/or METH2 protein. However, one skilled in the art could easily modify the exemplified studies to test the activity of METH1 and/or METH2 polynucleotides (e.g., gene therapy), agonists, and/or antagonists of METH1 and/or METH2.

### Example 43

### Effect of METH1 and/or METH2 on Cord Formation in Angiogenesis

Another step in angiogenesis is cord formation, marked by differentiation of endothelial cells. This bioassay measures the ability of microvascular endothelial cells to form capillary-like structures (hollow structures) when cultured in vitro.

CADMEC (microvascular endothelial cells) are purchased from Cell Applications, Inc. as proliferating (passage 2) cells and are cultured in Cell Applications' CADMEC Growth Medium and used at passage 5. For the in vitro angiogenesis assay, the wells of a 48-well cell culture plate are coated with Cell Applications' Attachment Factor Medium (200 ml/well) for 30 min. at 37° C. CADMEC are seeded onto the coated wells at 7,500 cells/well and cultured overnight in Growth Medium. The Growth Medium is then replaced with 300 mg Cell Applications' Chord Formation Medium containing control buffer or METH1 and/or METH2 (0.1 to 100 ng/ml) and the cells are cultured for an additional 48 hr. The numbers and lengths of the capillary-like chords are quantitated through use of the Boeckeler VIA-170 video image analyzer. All assays are done in triplicate.

Commercial (R&D) VEGF (50 ng/ml) is used as a positive control. b-esteradiol (1 ng/ml) is used as a negative control. The appropriate buffer (without protein) is also utilized as a control.

The studies described in this example tested activity in METH1 and/or METH2 protein. However, one skilled in the art could easily modify the exemplified studies to test the activity of METH1 and/or METH2 polynucleotides (e.g., gene therapy), agonists, and/or antagonists of METH1 and/or METH2.

### Example 44

### Rescue of Ischemia in Rabbit Lower Limb Model

To study the in vivo effects of METH1 and/or METH2 on ischemia, a rabbit hindlimb ischemia model is created by surgical removal of one femoral arteries as described previously (Takeshita, S. et al., Am J. Pathol 147:1649-1660 (1995)). The excision of the femoral artery results in retrograde propagation of thrombus and occlusion of the external iliac artery. Consequently, blood flow to the ischemic limb is dependent upon collateral vessels originating from the internal iliac artery (Takeshita, S. et al. Am J. Pathol 147:1649-1660 (1995)). An interval of 10 days is allowed for post-operative recovery of rabbits and development of endogenous collateral vessels. At 10 day post-operatively (day 0), after performing a baseline angiogram, the internal iliac artery of the ischemic limb is transfected with 500 mg naked METH1 and/or METH2 expression plasmid by arterial gene transfer technology using a hydrogel-coated balloon catheter as described (Riessen, R. et al. Hum Gene Ther. 4:749-758 (1993); Leclerc, G. et al. J. Clin. Invest. 90: 936-944 (1992)). When METH1 and/or METH2 is used in the treatment, a single bolus of 500 mg METH1 and/or METH2 protein or control is delivered into the internal iliac artery of the ischemic limb over a period of 1 min. through an infusion catheter. On day 30, various parameters are measured in these rabbits: (a) BP ratio—The blood pressure ratio of systolic pressure of the ischemic limb to that of normal limb; (b) Blood Flow and Flow Reserve—Resting FL: the blood flow during undilated condition and Max FL: the blood flow during fully dilated condition (also an indirect measure of the blood vessel amount) and Flow Reserve is reflected by the ratio of max FL: resting FL; (c) Angiographic Score—This is measured by the angiogram of collateral vessels. A score is determined by the percentage of circles in an overlaying grid that with crossing opacified arteries divided by the total number m the rabbit thigh; (d) Capillary density—The number of collateral capillaries determined in light microscopic sections taken from hindlimbs.

The studies described in this example tested activity in METH1 and/or METH2 protein. However, one skilled in the art could easily modify the exemplified studies to test the activity of METH1 and/or METH2 polynucleotides (e.g., gene therapy), agonists, and/or antagonists of METH1 and/or METH2.

### Example 45

### Effect of METH1 and/or METH2 on Vasodilation

Since dilation of vascular endothelium is important in reducing blood pressure, the ability of METH1 and/or METH2 to affect the blood pressure in spontaneously hypertensive rats (SHR) is examined. Increasing doses (0, 10, 30, 100, 300, and 900 mg/kg) of the METH1 and/or METH2 are administered to 13-14 week old spontaneously hypertensive rats (SHR). Data are expressed as the mean +/−SEM. Statistical analysis are performed with a paired t-test and statistical significance is defined as p<0.05 vs. the response to buffer alone.

The studies described in this example tested activity in METH1 and/or METH2 protein. However, one skilled in the art could easily modify the exemplified studies to test the activity of METH1 and/or METH2 polynucleotides (e.g., gene therapy), agonists, and/or antagonists of METH1 and/or METH2.

### Example 46

### Rat Ischemic Skin Flap Model

The evaluation parameters include skin blood flow, skin temperature, and factor VII immunohistochemistry or endothelial alkaline phosphatase reaction. METH1 and/or METH2 expression, during the skin ischemia, is studied using in situ hybridization.

The study in this model is divided into three parts as follows:

a) Ischemic skin

b) Ischemic skin wounds

c) Normal wounds

The experimental protocol includes:

a) Raising a 3×4 cm, single pedicle full-thickness random skin flap (myocutaneous flap over the lower back of the animal).

b) An excisional wounding (4-6 mm in diameter) in the ischemic skin (skin-flap).

c) Topical treatment with METH1 and/or METH2 of the excisional wounds (day 0, 1, 2, 3, 4 post-wounding) at the following various dosage ranges: 1 mg to 100 mg.

d) Harvesting the wound tissues at day 3, 5, 7, 10, 14 and 21 post-wounding for histological, immunohistochemical, and in situ studies.

The studies described in this example tested activity in METH1 and/or METH2 protein. However, one skilled in the art could easily modify the exemplified studies to test the activity of METH1 and/or METH2 polynucleotides (e.g., gene therapy), agonists, and/or antagonists of METH1 and/or METH2.

### Example 47

### Peripheral Arterial Disease Model

Angiogenic therapy using METH1 and/or METH2 is a novel therapeutic strategy to obtain restoration of blood flow around the ischemia in case of peripheral arterial diseases. The experimental protocol includes:

a) One side of the femoral artery is ligated to create ischemic muscle of the hindlimb, the other side of hindlimb serves as a control.

b) METH1 and/or METH2 protein, in a dosage range of 20 mg -500 mg, is delivered intravenously and/or intramuscularly 3 times (perhaps more) per week for 2-3 weeks.

c) The ischemic muscle tissue is collected after ligation of the femoral artery at 1, 2, and 3 weeks for the analysis of METH1 and/or METH2 expression and histology. Biopsy is also performed on the other side of normal muscle of the contralateral hindlimb.

The studies described in this example tested activity in METH1 and/or METH2 protein. However, one skilled in the art could easily modify the exemplified studies to test the activity of METH1 and/or METH2 polynucleotides (e.g., gene therapy), agonists, and/or antagonists of METH1 and/or METH2.

### Example 48

### Ischemic Myocardial Disease Model

METH1 and/or METH2 is evaluated as a potent mitogen capable of stimulating the development of collateral vessels, and restructuring new vessels after coronary artery occlusion. Alteration of METH1 and/or METH2 expression is investigated in situ. The experimental protocol includes:

a) The heart is exposed through a left-side thoracotomy in the rat. Immediately, the left coronary artery is occluded with a thin suture (6-0) and the thorax is closed.

b) METH1 and/or METH2 protein, in a dosage range of 20 mg -500 mg, is delivered intravenously and/or intramuscularly 3 times (perhaps more) per week for 24 weeks.

c) Thirty days after the surgery, the heart is removed and cross-sectioned for morphometric and in situ analyzes.

The studies described in this example tested activity in METH1 and/or METH2 protein. However, one skilled in the art could easily modify the exemplified studies to test the activity of METH1 and/or METH2 polynucleotides (e.g., gene therapy), agonists, and/or antagonists of METH1 and/or METH2.

### Example 49

### Rat Corneal Wound Healing Model

This animal model shows the effect of METH1 and/or METH2 on neovascularization. The experimental protocol includes:

a) Making a 1-1.5 mm long incision from the center of cornea into the stromal layer.

b) Inserting a spatula below the lip of the incision facing the outer corner of the eye.

c) Making a pocket (its base is 1-1.5 mm form the edge of the eye).

d) Positioning a pellet, containing 50 ng-5 ug of METH1 and/or METH2, within the pocket.

e) METH1 and/or METH2 treatment can also be applied topically to the corneal wounds in a dosage range of 20 mg -500 mg (daily treatment for five days).

The studies described in this example tested activity in METH1 and/or METH2 protein. However, one skilled in the art could easily modify the exemplified studies to test the activity of METH1 and/or METH2 polynucleotides (e.g., gene therapy), agonists, and/or antagonists of METH1 and/or METH2.

### Example 50

### Diabetic Mouse and Glucocorticoid-Impaired Wound Healing Models

A. Diabetic db+/db+ Mouse Model.

To demonstrate that METH1 and/or METH2 has an effect on the healing process, the genetically diabetic mouse model of wound healing is used. The full thickness wound healing model in the db+/db+ mouse is a well characterized, clinically relevant and reproducible model of impaired wound healing. Healing of the diabetic wound is dependent on formation of granulation tissue and re-epithelialization rather than contraction (Gartner, M. H. et al., J. Surg. Res. 52:389 (1992); Greenhalgh, D. G. et al., Am. J. Pathol. 136:1235 (1990)).

The diabetic animals have many of the characteristic features observed in Type II diabetes mellitus. Homozygous (db+/db+) mice are obese in comparison to their normal heterozygous (db+/+m) littermates. Mutant diabetic (db+/db+) mice have a single autosomal recessive mutation on chromosome 4 (db+) (Coleman et al. Proc. Natl. Acad. Sci. USA 77:283-293 (1982)). Animals show polyphagia, polydipsia and polyuria. Mutant diabetic mice (db+/db+) have elevated blood glucose, increased or normal insulin levels, and suppressed cell-mediated immunity (Mandel et al., J. Immunol. 120:1375 (1978); Debray-Sachs, M. et al., Clin. Exp. Immunol. 51(1):1-7 (1983); Leiter et al., Am. J. of Pathol. 114:46-55 (1985)). Peripheral neuropathy, myocardial complications, and microvascular lesions, basement membrane thickening and glomerular filtration abnormalities have been described in these animals (Norido, F. et al., Exp. Neurol. 83(2):221-232 (1984); Robertson et al., Diabetes 29(1):60-67 (1980); Giacomelli et al., Lab Invest. 40(4):460473 (1979); Coleman, D. L., Diabetes 31 (Suppl):1-6 (1982)). These homozygous diabetic mice develop hyperglycemia that is resistant to insulin analogous to human type II diabetes (Mandel et al., J. Immunol. 120:1375-1377 (1978)). The characteristics observed in these animals suggests that healing in this model may be similar to the healing observed in human diabetes (Greenhalgh, et al., Am. J. of Pathol. 136:1235-1246 (1990)).

Genetically diabetic female C57BL/KsJ (db+/db+) mice and their non-diabetic (db+/+m) heterozygous littermates are used in this study (Jackson Laboratories). The animals are purchased at 6 weeks of age and are 8 weeks old at the beginning of the study. Animals are individually housed and received food and water ad libitum. All manipulations are performed using aseptic techniques. The experiments are conducted according to the rules and guidelines of Human Genome Sciences, Inc. Institutional Animal Care and Use Committee and the Guidelines for the Care and Use of Laboratory Animals.

Wounding protocol is performed according to previously reported methods (Tsuboi, R. and Rifkin, D. B., J. Exp. Med. 172:245-251 (1990)). Briefly, on the day of wounding, animals are anesthetized with an intraperitoneal injection of Avertin (0.01 mg/mL), 2,2,2-tribromoethanol and 2-methyl-2-butanol dissolved in deionized water. The dorsal region of the animal is shaved and the skin washed with 70% ethanol solution and iodine. The surgical area is dried with sterile gauze prior to wounding. An 8 mm full-thickness wound is then created using a Keyes tissue punch. Immediately following wounding, the surrounding skin is gently stretched to eliminate wound expansion. The wounds are left open for the duration of the experiment. Application of the treatment is given topically for 5 consecutive days commencing on the day of wounding. Prior to treatment, wounds are gently cleansed with sterile saline and gauze sponges.

Wounds are visually examined and photographed at a fixed distance at the day of surgery and at two day intervals thereafter. Wound closure is determined by daily measurement on days 1-5 and on day 8. Wounds are measured horizontally and vertically using a calibrated Jameson caliper. Wounds are considered healed if granulation tissue is no longer visible and the wound is covered by a continuous epithelium.

METH1 and/or METH2 is administered using at a range different doses of METH1 and/or METH2, from 4 mg to 500 mg per wound per day for 8 days in vehicle. Vehicle control groups received 50 mL of vehicle solution.

Animals are euthanized on day 8 with an intraperitoneal injection of sodium pentobarbital (300 mg/kg). The wounds and surrounding skin are then harvested for histology and immunohistochemistry. Tissue specimens are placed in 10% neutral buffered formalin in tissue cassettes between biopsy sponges for further processing.

Three groups of 10 animals each (5 diabetic and 5 non-diabetic controls) are evaluated: 1) Vehicle placebo control, 2) METH1 and/or 3) METH2.

Wound closure is analyzed by measuring the area in the vertical and horizontal axis and obtaining the total square area of the wound. Contraction is then estimated by establishing the differences between the initial wound area (day 0) and that of post treatment (day 8). The wound area on day 1 is 64 mm², the corresponding size of the dermal punch. Calculations are made using the following formula:

[Open area on day 8]−[Open area on day 1]/[Open area on day 1]

Specimens are fixed in 10% buffered formalin and paraffin embedded blocks are sectioned perpendicular to the wound surface (5 mm) and cut using a Reichert-Jung microtome. Routine hematoxylin-eosin (H&E) staining is performed on cross-sections of bisected wounds. Histologic examination of the wounds are used to assess whether the healing process and the morphologic appearance of the repaired skin is altered by treatment with METH1 and/or METH2. This assessment included verification of the presence of cell accumulation, inflammatory cells, capillaries, fibroblasts, re-epithelialization and epidermal maturity (Greenhalgh, D. G. et al., Am. J. Pathol. 136:1235 (1990)). A calibrated lens micrometer is used by a blinded observer.

Tissue sections are also stained immunohistochemically with a polyclonal rabbit anti-human keratin antibody using ABC Elite detection system. Human skin is used as a positive tissue control while non-immune IgG is used as a negative control. Keratinocyte growth is determined by evaluating the extent of reepithelialization of the wound using a calibrated lens micrometer.

Proliferating cell nuclear antigen/cyclin (PCNA) in skin specimens is demonstrated by using anti-PCNA antibody (1:50) with an ABC Elite detection system. Human colon cancer served as a positive tissue control and human brain tissue is used as a negative tissue control. Each specimen included a section with omission of the primary antibody and substitution with non-immune mouse IgG. Ranking of these sections is based on the extent of proliferation on a scale of 0-8, the lower side of the scale reflecting slight proliferation to the higher side reflecting intense proliferation.

Experimental data are analyzed using an unpaired t test. A p value of <0.05 is considered significant.

B. Steroid Impaired Rat Model

The inhibition of wound healing by steroids has been well documented in various in vitro and in vivo systems (Wahl, S. M. Glucocorticoids and Wound healing. In: Anti-Inflammatory Steroid Action: Basic and Clinical Aspects. 280-302 (1989); Wahl, S. M. et al., J. Immunol. 115: 476-481 (1975); Werb, Z. et al., J. Exp. Med. 147:1684-1694 (1978)). Glucocorticoids retard wound healing by inhibiting angiogenesis, decreasing vascular permeability (Ebert, R. H., et al., An. Intern. Med. 37:701-705 (1952)), fibroblast proliferation, and collagen synthesis (Beck, L. S. et al., Growth Factors. 5: 295-304 (1991); Haynes, B. F. et al., J. Clin. Invest. 61: 703-797 (1978)) and producing a transient reduction of circulating monocytes (Haynes, B. F., et al., J. Clin. Invest. 61: 703-797 (1978); Wahl, S. M., “Glucocorticoids and wound healing”, In: Antiinflammatory Steroid Action: Basic and Clinical Aspects, Academic Press, New York, pp. 280-302 (1989)). The systemic administration of steroids to impaired wound healing is a well establish phenomenon in rats (Beck, L. S. et al., Growth Factors. 5: 295-304 (1991); Haynes, B. F., et al., J. Clin. Invest. 61: 703-797 (1978); Wahl, S. M., “Glucocorticoids and wound healing”, In: Antiinflammatory Steroid Action: Basic and Clinical Aspects, Academic Press, New York, pp. 280-302 (1989); Pierce, G. F. et al., Proc. Natl. Acad. Sci. USA 86: 2229-2233 (1989)).

To demonstrate that METH1 and/or METH2 has an effect on the healing process, the effects of multiple topical applications of METH1 and/or METH2 on full thickness excisional skin wounds in rats in which healing has been impaired by the systemic administration of methylprednisolone is assessed.

Young adult male Sprague Dawley rats weighing 250-300 g (Charles River Laboratories) are used in this example. The animals are purchased at 8 weeks of age and are 9 weeks old at the beginning of the study. The healing response of rats is impaired by the systemic administration of methylprednisolone (17 mg/kg/rat intramuscularly) at the time of wounding. Animals are individually housed and received food and water ad libitum. All manipulations are performed using aseptic techniques. This study is conducted according to the rules and guidelines of Human Genome Sciences, Inc. Institutional Animal Care and Use Committee and the Guidelines for the Care and Use of Laboratory Animals.

The wounding protocol is followed according to section A, above. On the day of wounding, animals are anesthetized with an intramuscular injection of ketamine (50 mg/kg) and xylazine (5 mg/kg). The dorsal region of the animal is shaved and the skin washed with 70% ethanol and iodine solutions. The surgical area is dried with sterile gauze prior to wounding. An 8 mm full-thickness wound is created using a Keyes tissue punch. The wounds are left open for the duration of the experiment. Applications of the testing materials are given topically once a day for 7 consecutive days commencing on the day of wounding and subsequent to methylprednisolone administration. Prior to treatment, wounds are gently cleansed with sterile saline and gauze sponges.

Wounds are visually examined and photographed at a fixed distance at the day of wounding and at the end of treatment. Wound closure is determined by daily measurement on days 1-5 and on day 8. Wounds are measured horizontally and vertically using a calibrated Jameson caliper. Wounds are considered healed if granulation tissue is no longer visible and the wound is covered by a continuous epithelium. METH1 and/or METH2 is administered using at a range different doses of METH1 and/or METH2, from 4 mg to 500 mg per wound per day for 8 days in vehicle. Vehicle control groups received 50 mL of vehicle solution.

Animals are euthanized on day 8 with an intraperitoneal injection of sodium pentobarbital (300 mg/kg). The wounds and surrounding skin are then harvested for histology. Tissue specimens are placed in 10% neutral buffered formalin in tissue cassettes between biopsy sponges for further processing.

Four groups of 10 animals each (5 with methylprednisolone and 5 without glucocorticoid) are evaluated: 1) Untreated group 2) Vehicle placebo control 3) METH1 and 4) METH2 treated groups.

Wound closure is analyzed by measuring the area in the vertical and horizontal axis and obtaining the total area of the wound. Closure is then estimated by establishing the differences between the initial wound area (day 0) and that of post treatment (day 8). The wound area on day 1 is 64 mm², the corresponding size of the dermal punch. Calculations are made using the following formula:

[Open area on day 8]−[Open area on day 1]/[Open area on day 1]

Specimens are fixed in 10% buffered formalin and paraffin embedded blocks are sectioned perpendicular to the wound surface (5 mm) and cut using an Olympus microtome. Routine hematoxylin-eosin (H&E) staining is performed on cross-sections of bisected wounds. Histologic examination of the wounds allows assessment of whether the healing process and the morphologic appearance of the repaired skin is improved by treatment with METH1 and/or METH2. A calibrated lens micrometer is used by a blinded observer to determine the distance of the wound gap.

Experimental data are analyzed using an unpaired t test. A p value of <0.05 is considered significant.

The studies described in this example tested activity in METH1 and/or METH2 protein. However, one skilled in the art could easily modify the exemplified studies to test the activity of METH1 and/or METH2 polynucleotides (e.g., gene therapy), agonists, and/or antagonists of METH1 and/or METH2.

### Example 51

### Lymphadema Animal Model

The purpose of this experimental approach is to create an appropriate and consistent lymphedema model for testing the therapeutic effects of METH1 and/or METH2 in lymphangiogenesis and re-establishment of the lymphatic circulatory system in the rat hind limb. Effectiveness is measured by swelling volume of the affected limb, quantification of the amount of lymphatic vasculature, total blood plasma protein, and histopathology. Acute lymphedema is observed for 7-10 days. Perhaps more importantly, the chronic progress of the edema is followed for up to 34 weeks.

Prior to beginning surgery, blood sample is drawn for protein concentration analysis. Male rats weighing approximately ˜350 g are dosed with Pentobarbital. Subsequently, the right legs are shaved from knee to hip. The shaved area is swabbed with gauze soaked in 70% EtOH. Blood is drawn for serum total protein testing. Circumference and volumetric measurements are made prior to injecting dye into paws after marking 2 measurement levels (0.5 cm above heel, at mid-pt of dorsal paw). The intradermal dorsum of both right and left paws are injected with 0.05 ml of 1% Evan's Blue. Circumference and volumetric measurements are then made following injection of dye into paws.

Using the knee joint as a landmark, a mid-leg inguinal incision is made circumferentially allowing the femoral vessels to be located. Forceps and hemostats are used to dissect and separate the skin flaps. After locating the femoral vessels, the lymphatic vessel that runs along side and underneath the vessel(s) is located. The main lymphatic vessels in this area are then electrically coagulated or suture ligated.

Using a microscope, muscles in back of the leg (near the semitendinosis and adductors) are bluntly dissected. The popliteal lymph node is then located. The 2 proximal and 2 distal lymphatic vessels and distal blood supply of the popliteal node are then and ligated by suturing. The popliteal lymph node, and any accompanying adipose tissue, is then removed by cutting connective tissues.

Care is taken to control any mild bleeding resulting from this procedure. After lymphatics are occluded, the skin flaps are sealed by using liquid skin (Vetbond) (A J Buck). The separated skin edges are sealed to the underlying muscle tissue while leaving a gap of ˜0.5 cm around the leg. Skin also may be anchored by suturing to underlying muscle when necessary.

To avoid infection, animals are housed individually with mesh (no bedding). Recovering animals are checked daily through the optimal edematous peak, which typically occurred by day 5-7. The plateau edematous peak are then observed. To evaluate the intensity of the lymphedema, the circumference and volumes of 2 designated places on each paw before operation and daily for 7 days are measured. The effect plasma proteins on lymphedema is determined and whether protein analysis is a useful testing perimeter is also investigated. The weights of both control and edematous limbs are evaluated at 2 places. Analysis is performed in a blind manner.

Circumference Measurements: Under brief gas anesthetic to prevent limb movement, a cloth tape is used to measure limb circumference. Measurements are done at the ankle bone and dorsal paw by 2 different people then those 2 readings are averaged. Readings are taken from both control and edematous limbs.

Volumetric Measurements: On the day of surgery, animals are anesthetized with Pentobarbital and are tested prior to surgery. For daily volumetrics animals are under brief halothane anesthetic (rapid immobilization and quick recovery), both legs are shaved and equally marked using waterproof marker on legs. Legs are first dipped in water, then dipped into instrument to each marked level then measured by Buxco edema software(Chen/Victor). Data is recorded by one person, while the other is dipping the limb to marked area.

Blood-plasma protein measurements: Blood is drawn, spun, and serum separated prior to surgery and then at conclusion for total protein and Ca2+comparison.

Limb Weight Comparison: After drawing blood, the animal is prepared for tissue collection. The limbs are amputated using a quillitine, then both experimental and control legs are cut at the ligature and weighed. A second weighing is done as the tibio-cacaneal joint is disarticulated and the foot is weighed.

Histological Preparations: The transverse muscle located behind the knee (popliteal) area is dissected and arranged in a metal mold, filled with freezeGel, dipped into cold methylbutane, placed into labeled sample bags at −80EC until sectioning. Upon sectioning, the muscle is observed under fluorescent microscopy for lymphatics.

The studies described in this example tested activity in METH1 and/or METH2 protein. However, one skilled in the art could easily modify the exemplified studies to test the activity of METH1 and/or METH2 polynucleotides (e.g., gene therapy), agonists, and/or antagonists of METH1 and/or METH2.

### Example 52

### Generation of Constructs and Expression of METH1

Two constructs having either a Flag peptide sequence or a human IgG1 Fc domain fused to the full-length METH1 gene at its C-terminus were generated, using methods well known in the art. The construct names, pFlag-CMV-5a:METH1 (ID 822) and pC4Fc:METH1 (ID 821) were assigned.

The following primers were used for pFlag-CMV-5a:METH1:

(SEQ ID NO:128)

5′: AAGAATGCGGCCGCAGCCACCATGGGAACGCGGAGCGGGCTCC

(SEQ ID NO:129)

3′: GATCGCGGTACCACTGCATTCTGCCATTGTGCAAAAGTCTATG

METH1 was amplified using the indicated primers, and digested with Asp718. The vector pFLAGCMV-5a was also digested with Asp718. The resulting restriction products were ligated together.

The following primers were used for pC4Fc:METH1:

(SEQ ID NO:130)

5′: GATCTATGATCAGCCACCATGGGGAACGCGGAGCGGGCTCC

(SEQ ID NO:131)

3′: GACTGCTCTAGAACTGCATTCTGCCATTGTGCAAAAGTCTATG

METH1 was amplified using the indicated primers, and digested with BcII and Xba. The vector pC4Fc was also digested with BcII and Xba. The resulting restriction products were ligated together.

Constructs pA2gp:METH1(H542-Q894).Fc and pA2gp:METH1(H542-Q894) can also be made.

Also, pC4Fc:Methl.M1-P799 can be made using the following primers:

| | | |
|---|---|---|
| 5′primer: | GATCTA TGATCA GCCACCATGGGGAACGCGGAGCGGGCTCC | (SEQ ID NO:132) |
| 3′primer: | GCGTGC TCTAGA AGGGCTAAAGCTGCGAATTC | (SEQ ID NO:133) |

METH1 is amplified using the indicated primers, and digested with BclI and Xba The vector pC4Fc was also digested with BclI and Xba, and ligated to the digested METH1 fragment.

pFLAG-CMV-1:Meth1.F236-E614 can be made using the following primers:

| | |
|---|---|
| 5′ primer: | |
| GTACCC AAGCTT TTTGTGTCCAGTCACCGC | (SEQ ID NO:134) |
| 3′ primer: | |
| GCGTGC TCTAGA TTACTCGTTGTGTGCTTCAC | (SEQ ID NO:135) |

METH1 is amplified using the indicated primers and digested with HindIII and Xba. The vector pFLAG-CMV-1 is also digested with HindIII and Xba and ligated to the digested METH1 fragment.

The constructs were made in order to confirm the anti-angiogenesis activity of METH1. The full length METH1 gene was PCR cloned into pC4Fc and pFlagCMV5a vectors. Both pC4Fc:METH1 and pFLAGCMV5a:METH1 were obtained and the sequence confirmed.

Transient transfections on 293T cells were done using lipofectamine plus (LTI) reagent and held for production under serum-free conditions. Western analysis was done with either anti-huFc Ab or anti-Flag M2 Ab. METH1-Fc conditioned media showed at least five bands with varying degree of intensity. Their estimated MWs are 130-140 kD(weak), 110-120 kD(weak),52 kD(strong),45-48 kD(strong) and 32-35 kD(strongest). Two weaker bands at about 60 and 90 kD were also detectable. METH1-Flag conditioned medium revealed three major bands with equal intensity. They are about 100-110 kD, 70-80 kD and 22 kD. Transient transfection of METH1-Fc in 293T cells. A second batch of METH1-Fc protein was produced in medium with 1% serum as described above.

5.5 day conditioned medium from transiently transfected cells was run on a ProteinA column and eluted. The fractions containing protein were examined by SDS-PAGE under reduced and non-reduced conditions and stained with Coomassie Blue. A second gel was also prepared for N-terminal sequence analysis.

197 μg of protein were recovered which demonstrated 4 bands under reducing conditions. Three of the bands were strong, one was weak. N-terminal sequencing of the bands suggested that 2 of the bands contained proteins with a blocked N-termini. Of the 2 bands giving sequence, one was an Fc-derived fragment, the other a cleavage product of the METH1.Fc fusion starting at L800 (containing two of the thrombospondin-like domains). This suggests that METH1 is processed with at least 2 cleavage sites (possibly more) since only the C-terminal fragments still linked to the Fc fragment would be purified on the Protein A column.

The transfected 293T cells were conditioned in medium containing 1% dialyzed, low IgG, fetal calf serum to attempt to decrease the proteolysis of the recombinant secreted protein. The purification and analysis was as described above. The yield of protein was significantly higher than the first batch, possibly reflecting the effect of the serum in the medium. While some processing may have been slowed by the serum, the majority of the protein remained approximately 31 kD on a reducing gel.

N-terminal sequencing of resolved bands under reducing conditions indicated the protein is processed at L800 of the 950 residue METH1 orf, with other possible cleavage occurring N-terminal to this site. The observed cleavage site was considered unusual since it followed a Pro. A total of 197.4 μg of protein was isolated (HGi2100-D293T1). Analysis of flag protein (pFlag-CMV-5a:METH1), consisting of at least three bands on the Western blot (120, 97 and 21 Kd) indicated that only one band (21 kd) could be confirmed as METH1 and the other bands were of non-METH1 origin.

Since sequencing of the purified METH1 Fc protein suggested an unusual cleavage site, a second batch of METH1 Fc was prepared with cells grown in 1% FBS, to possibly inhibit undesirable processing. A preliminary assessment of the product suggests that no difference in processing resulted from the change in medium, but protein yields were increased.

Functional assays of the initial Fc and Flag protein supernatants performed included proliferation of Human Microvascular Endothelial Cells (HVECs) and in vitro cord formation using Bovine aortic endothelial cells (BAECs). The proliferation assay indicated increased rates of HMEC proliferation in response to both culture supernatants, which may be attributable to high background stimulation from the conditioned medium. Cord formation assays of both the Fc and Flag supernatants indicated inhibition of cord formation relative to a medium/collagen control in two independent experiments.

### Example 53

### In vitro activity of METH1

Proliferation

HMVECs were used in an alamar blue assay to determine if METH1 supernatants have functional anti-angiogenic activity, detectable by an inhibition of EC proliferation. FGF-2 was used as the primary stimulus for proliferation and culture supernatants were used at a 1:4 final dilution. The proliferation assays indicated significantly increased rates of HMVEC proliferation in response to both culture supernatants, which may be attributable to high background stimulation from the conditioned medium. This problem should be reduced or eliminated by the use of purified proteins.

Cord Formation

The addition of soluble type I collagen to endothelial cells and the appropriate growth factors will induce the production of tube-like structures or cords of endothelial cells in culture which involves both the migration of endothelial cells and the selective deletion (apoptosis) of cells not involved in these structures. Bovine aortic endothelial cells (BAECs) were used to detect inhibition of stable cord formation when cultured with METH1-Fc and METH1-Flag containing culture supernatants at a 1:4 dilution. Qualitative assessment of the cord formation indicated inhibition with both of the tested supernatants relative to the collagen-treated control. However, a non-matched conditioned medium control also generated inhibition of cord formation, suggesting that non-specific cellular toxicity might also contribute to the observed inhibition.

The studies described in this example tested activity in METH1 protein. However, one skilled in the art could easily modify the exemplified studies to test the activity of METH2 polypeptides, METH1 and/or METH2 polynucleotides (e.g., gene therapy), agonists, and/or antagonists of METH1 and/or METH2.

It will be clear that the invention may be practiced otherwise than as particularly described in the foregoing description and examples.

Numerous modifications and variations of the present invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

What is claimed is:

1. A method for treating an individual, comprising administering an effective amount of METH1 or METH2, wherein said method is used to treat benign tumors, an ocular angiogenic disease, vasculogenesis, granulations, hypertrophic scars, nonunion fractures, scleroderma, trachoma, vascular adhesions, myocardial angiogenesis, coronary collaterals, cerebral collaterals, arteriovenous malformations, ischemic limb angiogenesis, Osler-Webber Syndrom, plaque neovascularization, hemophiliac joints, angiofibroma, fibromuscular dysplasia, wound granulation, or atherosclerosis.

2. A method for treating an individual, comprising administering an effective amount of METH1 or METH2, wherein said method is used in birth control.

3. The method of claim 1, further comprising administering another angiogenic compound.

4. The method of claim 1, wherein said METH1 or METH2 is administered by gene therapy means wherein cells have been modified to produce and secrete METH1 or METH2.
